SEQUENCE LISTING (2613 sequences)

SEQ ID NO: 1 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 2 - protein, 11 aa, Homo sapiens
TSEKRPFMCAY

SEQ ID NO: 3 - protein, 11 aa, Homo sapiens
STVTFDGTPSY

SEQ ID NO: 4 - protein, 11 aa, Homo sapiens
HTTPILCGAQY

SEQ ID NO: 5 - protein, 11 aa, Homo sapiens
ESQPAIRNQGY

SEQ ID NO: 6 - protein, 11 aa, Homo sapiens
GSQALLLRTPY

SEQ ID NO: 7 - protein, 11 aa, Homo sapiens
HSRKHTGEKPY

SEQ ID NO: 8 - protein, 10 aa, Homo sapiens
FTGTAGACRY

SEQ ID NO: 9 - protein, 10 aa, Homo sapiens
RTPYSSDNLY

SEQ ID NO: 10 - protein, 10 aa, Homo sapiens
TTPILCGAQY

SEQ ID NO: 11 - protein, 9 aa, Homo sapiens
VTFDGTPSY

SEQ ID NO: 12 - protein, 10 aa, Homo sapiens
SLGGGGGCAL

SEQ ID NO: 13 - protein, 10 aa, Homo sapiens
NALLPAVPSL

SEQ ID NO: 14 - protein, 10 aa, Homo sapiens
AIRNQGYSTV

SEQ ID NO: 15 - protein, 10 aa, Homo sapiens
NMHQRNMTKL

SEQ ID NO: 16 - protein, 9 aa, Homo sapiens
ALLPAVPSL

SEQ ID NO: 17 - protein, 9 aa, Homo sapiens
DLNALLPAV

SEQ ID NO: 18 - protein, 9 aa, Homo sapiens
SLGEQQYSV

SEQ ID NO: 19 - protein, 9 aa, Homo sapiens
NLGATLKGV

SEQ ID NO: 20 - protein, 9 aa, Homo sapiens
NLYQMTSQL

SEQ ID NO: 21 - protein, 9 aa, Homo sapiens
ILCGAQYRI

SEQ ID NO: 22 - protein, 10 aa, Homo sapiens
DVRRVPGVAP

SEQ ID NO: 23 - protein, 10 aa, Homo sapiens
ALLPAVPSLG

SEQ ID NO: 24 - protein, 10 aa, Homo sapiens
ALPVSGAAQW

SEQ ID NO: 25 - protein, 10 aa, Homo sapiens
AIRNQGYSTV

SEQ ID NO: 26 - protein, 10 aa, Homo sapiens
RHQRRHTGVK

SEQ ID NO: 27 - protein, 10 aa, Homo sapiens
GVFRGIQDVR

SEQ ID NO: 28 - protein, 9 aa, Homo sapiens
RVPGVAPTL

SEQ ID NO: 29 - protein, 9 aa, Homo sapiens
RIHTHGVFR

SEQ ID NO: 30 - protein, 9 aa, Homo sapiens
DVRRVPGVA

SEQ ID NO: 31 - protein, 9 aa, Homo sapiens
HQRRHTGVK

SEQ ID NO: 32 - protein, 10 aa, Homo sapiens
CTGSQALLLR

SEQ ID NO: 33 - protein, 10 aa, Homo sapiens
GVFRGIQDVR

SEQ ID NO: 34 - protein, 10 aa, Homo sapiens
HTGVKPFQCK

SEQ ID NO: 35 - protein, 10 aa, Homo sapiens
RTHTGKTSEK

SEQ ID NO: 36 - protein, 10 aa, Homo sapiens
KTHTRTHTGK

SEQ ID NO: 37 - protein, 10 aa, Homo sapiens
RSASETSEKR

SEQ ID NO: 38 - protein, 10 aa, Homo sapiens
LSHLQMHSRK

SEQ ID NO: 39 - protein, 10 aa, Homo sapiens
FSCRWPSCQK

SEQ ID NO: 40 - protein, 9 aa, Homo sapiens
RSASETSEK

SEQ ID NO: 41 - protein, 9 aa, Homo sapiens
FSRSDQLKR

SEQ ID NO: 42 - protein, 10 aa, Homo sapiens
AYPGCNKRYF

SEQ ID NO: 43 - protein, 10 aa, Homo sapiens
QYRIHTHGVF

SEQ ID NO: 44 - protein, 10 aa, Homo sapiens
AFTVHFSGQF

SEQ ID NO: 45 - protein, 10 aa, Homo sapiens
PPPPPPPHSF

SEQ ID NO: 46 - protein, 10 aa, Homo sapiens
PPPPPPHSFI

SEQ ID NO: 47 - protein, 10 aa, Homo sapiens
PYLPSCLESQ

SEQ ID NO: 48 - protein, 9 aa, Homo sapiens
DFKDCERRF

SEQ ID NO: 49 - protein, 9 aa, Homo sapiens
GCNKRYFKL

SEQ ID NO: 50 - protein, 9 aa, Homo sapiens
ALLPAVPSL

SEQ ID NO: 51 - protein, 9 aa, Homo sapiens
PPPPPPHSF

SEQ ID NO: 52 - protein, 10 aa, Homo sapiens
TVTFDGTPSY

SEQ ID NO: 53 - protein, 10 aa, Homo sapiens
DFAPPGASAY

SEQ ID NO: 54 - protein, 10 aa, Homo sapiens
EGQSNHSTGY

SEQ ID NO: 55 - protein, 10 aa, Homo sapiens
TTPILCGAQY

SEQ ID NO: 56 - protein, 10 aa, Homo sapiens
ETSEKRPFMC

SEQ ID NO: 57 - protein, 9 aa, Homo sapiens
DVRDLNALL

SEQ ID NO: 58 - protein, 9 aa, Homo sapiens
VTFDGTPSY

SEQ ID NO: 59 - protein, 9 aa, Homo sapiens
FTVHFSGQF

SEQ ID NO: 60 - protein, 9 aa, Homo sapiens
EKRPFMCAY

SEQ ID NO: 61 - protein, 9 aa, Homo sapiens
ETSEKRPFM

SEQ ID NO: 62 - protein, 11 aa, Homo sapiens
GVFRGIQDVRR

SEQ ID NO: 63 - protein, 11 aa, Homo sapiens
TTPILCGAQYR

SEQ ID NO: 64 - protein, 11 aa, Homo sapiens
ELVRHHNMHQR

SEQ ID NO: 65 - protein, 11 aa, Homo sapiens
PSCLESQPAIR

SEQ ID NO: 66 - protein, 10 aa, Homo sapiens
CTGSQALLLR

SEQ ID NO: 67 - protein, 10 aa, Homo sapiens
GVFRGIQDVR

SEQ ID NO: 68 - protein, 10 aa, Homo sapiens
KTHTRTHTGK

SEQ ID NO: 69 - protein, 10 aa, Homo sapiens
LVRHHNMHQR

SEQ ID NO: 70 - protein, 9 aa, Homo sapiens
FTGTAGACR

SEQ ID NO: 71 - protein, 9 aa, Homo sapiens
RIHTHGVFR

SEQ ID NO: 72 - protein, 10 aa, Homo sapiens
PPGASAYGSL

SEQ ID NO: 73 - protein, 10 aa, Homo sapiens
EPHEEQCLSA

SEQ ID NO: 74 - protein, 10 aa, Homo sapiens
LPSCLESQPA

SEQ ID NO: 75 - protein, 10 aa, Homo sapiens
PPPPPPHSFI

SEQ ID NO: 76 - protein, 10 aa, Homo sapiens
PPSQASSGQA

SEQ ID NO: 77 - protein, 9 aa, Homo sapiens
DPMGQQGSL

SEQ ID NO: 78 - protein, 9 aa, Homo sapiens
PPPPPHSFI

SEQ ID NO: 79 - protein, 9 aa, Homo sapiens
PPPPPPHSF

SEQ ID NO: 80 - protein, 9 aa, Homo sapiens
TPSHHAAQF

SEQ ID NO: 81 - protein, 9 aa, Homo sapiens
WPSCQKKFA

SEQ ID NO: 82 - protein, 9 aa, Homo sapiens
KRYFKLSHL

SEQ ID NO: 83 - protein, 9 aa, Homo sapiens
GCNKRYFKL

SEQ ID NO: 84 - protein, 9 aa, Homo sapiens
KKFARSDEL

SEQ ID NO: 85 - protein, 9 aa, Homo sapiens
GATLKGVAA

SEQ ID NO: 86 - protein, 9 aa, Homo sapiens
RRFSRSDQL

SEQ ID NO: 87 - protein, 8 aa, Homo sapiens
MTKLQLAL

SEQ ID NO: 88 - protein, 8 aa, Homo sapiens
EPHEEQCL

SEQ ID NO: 89 - protein, 8 aa, Homo sapiens
ETSEKRPF

SEQ ID NO: 90 - protein, 8 aa, Homo sapiens
CNKRYFKL

SEQ ID NO: 91 - protein, 8 aa, Homo sapiens
RNMTKLQL

SEQ ID NO: 92 - protein, 10 aa, Homo sapiens
QQYSVPPPVY

SEQ ID NO: 93 - protein, 10 aa, Homo sapiens
TVTFDGTPSY

SEQ ID NO: 94 - protein, 10 aa, Homo sapiens
QQGSLGEQQY

SEQ ID NO: 95 - protein, 10 aa, Homo sapiens
SQALLLRTPY

SEQ ID NO: 96 - protein, 10 aa, Homo sapiens
SQPAIRNQGY

SEQ ID NO: 97 - protein, 10 aa, Homo sapiens
FQCKTCQRKF

SEQ ID NO: 98 - protein, 9 aa, Homo sapiens
AQWAPVLDF

SEQ ID NO: 99 - protein, 9 aa, Homo sapiens
GQSNHSTGY

SEQ ID NO: 100 - protein, 9 aa, Homo sapiens
NQGYSTVTF

SEQ ID NO: 101 - protein, 9 aa, Homo sapiens
CLSAFTVHF

SEQ ID NO: 102 - protein, 9 aa, Homo sapiens
HEEQCLSAF

SEQ ID NO: 103 - protein, 9 aa, Homo sapiens
SETSEKRPF

SEQ ID NO: 104 - protein, 9 aa, Homo sapiens
GEKPYQCDF

SEQ ID NO: 105 - protein, 9 aa, Homo sapiens
SEKPFSCRW

SEQ ID NO: 106 - protein, 9 aa, Homo sapiens
AEPHEEQCL

SEQ ID NO: 107 - protein, 9 aa, Homo sapiens
DVRDLNALL

SEQ ID NO: 108 - protein, 9 aa, Homo sapiens
QALLLRTPY

SEQ ID NO: 109 - protein, 8 aa, Homo sapiens
EEQCLSAF

SEQ ID NO: 110 - protein, 8 aa, Homo sapiens
ETSEKRPF

SEQ ID NO: 111 - protein, 8 aa, Homo sapiens
DELVRHHN

SEQ ID NO: 112 - protein, 10 aa, Homo sapiens
RRVPGVAPTL

SEQ ID NO: 113 - protein, 10 aa, Homo sapiens
RRFSRSDQLK

SEQ ID NO: 114 - protein, 10 aa, Homo sapiens
CRWPSCQKKF

SEQ ID NO: 115 - protein, 10 aa, Homo sapiens
LRTPYSSDNL

SEQ ID NO: 116 - protein, 9 aa, Homo sapiens
RRFSRSDQL

SEQ ID NO: 117 - protein, 9 aa, Homo sapiens
KRYFKLSHL

SEQ ID NO: 118 - protein, 9 aa, Homo sapiens
RRHTGVKPF

SEQ ID NO: 119 - protein, 9 aa, Homo sapiens
FRGIQDVRR

SEQ ID NO: 120 - protein, 9 aa, Homo sapiens
CRWPSCQKK

SEQ ID NO: 121 - protein, 9 aa, Homo sapiens
ARSDELVRH

SEQ ID NO: 122 - protein, 10 aa, Homo sapiens
PPGASAYGSL

SEQ ID NO: 123 - protein, 10 aa, Homo sapiens
PPPPPPPHSF

SEQ ID NO: 124 - protein, 10 aa, Homo sapiens
PPPPPPHSFI

SEQ ID NO: 125 - protein, 9 aa, Homo sapiens
TPYSSDNLY

SEQ ID NO: 126 - protein, 9 aa, Homo sapiens
QPAIRNQGY

SEQ ID NO: 127 - protein, 9 aa, Homo sapiens
DPMGQQGSL

SEQ ID NO: 128 - protein, 9 aa, Homo sapiens
TPILCGAQY

SEQ ID NO: 129 - protein, 9 aa, Homo sapiens
TPSHHAAQF

SEQ ID NO: 130 - protein, 9 aa, Homo sapiens
PPPPPPHSF

SEQ ID NO: 131 - protein, 9 aa, Homo sapiens
YPGCNKRYF

SEQ ID NO: 132 - protein, 10 aa, Homo sapiens
ASETSEKRPF

SEQ ID NO: 133 - protein, 10 aa, Homo sapiens
QASSGQARMF

SEQ ID NO: 134 - protein, 10 aa, Homo sapiens
RTPYSSDNLY

SEQ ID NO: 135 - protein, 10 aa, Homo sapiens
DSCTGSQALL

SEQ ID NO: 136 - protein, 9 aa, Homo sapiens
ASSGQARMF

SEQ ID NO: 137 - protein, 9 aa, Homo sapiens
RVPGVAPTL

SEQ ID NO: 138 - protein, 9 aa, Homo sapiens
TSQLECMTW

SEQ ID NO: 139 - protein, 9 aa, Homo sapiens
HTHGVFRGI

SEQ ID NO: 140 - protein, 9 aa, Homo sapiens
RTPYSSDNL

SEQ ID NO: 141 - protein, 9 aa, Homo sapiens
RSDELVRHH

SEQ ID NO: 142 - protein, 15 aa, Homo sapiens
ASAYGSLGGPAPPPA

SEQ ID NO: 143 - protein, 15 aa, Homo sapiens
GSDVRDLNALLPAVP

SEQ ID NO: 144 - protein, 15 aa, Homo sapiens
IQDVRRVPGVAPTLV

SEQ ID NO: 145 - protein, 15 aa, Homo sapiens
VRDLNALLPAVPSLG

SEQ ID NO: 146 - protein, 15 aa, Homo sapiens
GATLKGVAAGSSSSV

SEQ ID NO: 147 - protein, 15 aa, Homo sapiens
TVHFSGQFTGTAGAC

SEQ ID NO: 148 - protein, 15 aa, Homo sapiens
VRRVPGVAPTLVRSA

SEQ ID NO: 149 - protein, 15 aa, Homo sapiens
NKRYFKLSHLQMHSR

SEQ ID NO: 150 - protein, 15 aa, Homo sapiens
LPAVPSLGGGGGCAL

SEQ ID NO: 151 - protein, 15 aa, Homo sapiens
RDLNALLPAVPSLGG

SEQ ID NO: 152 - protein, 15 aa, Homo sapiens
YSTVTFDGTPSYGHT

SEQ ID NO: 153 - protein, 15 aa, Homo sapiens
MGSDVRDLNALLPAV

SEQ ID NO: 154 - protein, 15 aa, Homo sapiens
YQCDFKDCERRFSRS

SEQ ID NO: 155 - protein, 15 aa, Homo sapiens
VPSLGGGGGCALPVS

SEQ ID NO: 156 - protein, 15 aa, Homo sapiens
VLDFAPPGASAYGSL

SEQ ID NO: 157 - protein, 15 aa, Homo sapiens
LYQMTSQLECMTWNQ

SEQ ID NO: 158 - protein, 15 aa, Homo sapiens
PTLVRSASETSEKRP

SEQ ID NO: 159 - protein, 15 aa, Homo sapiens
HHNMHQRNMTKLQLA

SEQ ID NO: 160 - protein, 15 aa, Homo sapiens
NKRYFKLSHLQMHSR

SEQ ID NO: 161 - protein, 15 aa, Homo sapiens
TVHFSGQFTGTAGAC

SEQ ID NO: 162 - protein, 15 aa, Homo sapiens
ARMFPNAPYLPSCLE

SEQ ID NO: 163 - protein, 15 aa, Homo sapiens
NQGYSTVTFDGTPSY

SEQ ID NO: 164 - protein, 15 aa, Homo sapiens
TPSYGHTPSHHAAQF

SEQ ID NO: 165 - protein, 15 aa, Homo sapiens
NHSFKHEDPMGQQGS

SEQ ID NO: 166 - protein, 15 aa, Homo sapiens
RTPYSSDNLYQMTSQ

SEQ ID NO: 167 - protein, 15 aa, Homo sapiens
SVKWTEGQSNHSTGY

SEQ ID NO: 168 - protein, 15 aa, Homo sapiens
STGYESDNHTTPILC

SEQ ID NO: 169 - protein, 15 aa, Homo sapiens
KRPFMCAYPGCNKRY

SEQ ID NO: 170 - protein, 15 aa, Homo sapiens
TPSYGHTPSHHAAQF

SEQ ID NO: 171 - protein, 15 aa, Homo sapiens
TVTFDGTPSYGHTPS

SEQ ID NO: 172 - protein, 15 aa, Homo sapiens
LSAFTVHFSGQFTGT

SEQ ID NO: 173 - protein, 15 aa, Homo sapiens
TPTDSCTGSQALLLR

SEQ ID NO: 174 - protein, 15 aa, Homo sapiens
LKGVAAGSSSSVKWT

SEQ ID NO: 175 - protein, 15 aa, Homo sapiens
TVHFSGQFTGTAGAC

SEQ ID NO: 176 - protein, 15 aa, Homo sapiens
YSTVTFDGTPSYGHT

SEQ ID NO: 177 - protein, 15 aa, Homo sapiens
CGAQYRIHTHGVFRG

SEQ ID NO: 178 - protein, 15 aa, Homo sapiens
HGVFRGIQDVRRVPG

SEQ ID NO: 179 - protein, 15 aa, Homo sapiens
APTLVRSASETSEKR

SEQ ID NO: 180 - protein, 15 aa, Homo sapiens
FRGIQDVRRVPGVAP

SEQ ID NO: 181 - protein, 15 aa, Homo sapiens
NKRYFKLSHLQMHSR

SEQ ID NO: 182 - protein, 15 aa, Homo sapiens
QCDFKDCERRFSRSD

SEQ ID NO: 183 - protein, 15 aa, Homo sapiens
STGYESDNHTTPILC

SEQ ID NO: 184 - protein, 15 aa, Homo sapiens
SCRWPSCQKKFARSD

SEQ ID NO: 185 - protein, 15 aa, Homo sapiens
AAQWAPVLDFAPPGA

SEQ ID NO: 186 - protein, 15 aa, Homo sapiens
ASAYGSLGGPAPPPA

SEQ ID NO: 187 - protein, 15 aa, Homo sapiens
PGVAPTLVRSASETS

SEQ ID NO: 188 - protein, 15 aa, Homo sapiens
QMNLGATLKGVAAGS

SEQ ID NO: 189 - protein, 15 aa, Homo sapiens
WAPVLDFAPPGASAY

SEQ ID NO: 190 - protein, 15 aa, Homo sapiens
RPFMCAYPGCNKRYF

SEQ ID NO: 191 - protein, 15 aa, Homo sapiens
GSDVRDLNALLPAVP

SEQ ID NO: 192 - protein, 15 aa, Homo sapiens
NALLPAVPSLGGGGG

SEQ ID NO: 193 - protein, 15 aa, Homo sapiens
PPGASAYGSLGGPAP

SEQ ID NO: 194 - protein, 15 aa, Homo sapiens
EQCLSAFTVHFSGQF

SEQ ID NO: 195 - protein, 15 aa, Homo sapiens
TAGACRYGPFGPPPP

SEQ ID NO: 196 - protein, 15 aa, Homo sapiens
PSCLESQPAIRNQGY

SEQ ID NO: 197 - protein, 15 aa, Homo sapiens
WNQMNLGATLKGVAA

SEQ ID NO: 198 - protein, 15 aa, Homo sapiens
IQDVRRVPGVAPTLV

SEQ ID NO: 199 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 200 - protein, 11 aa, Homo sapiens
LTDVSPEPLQA

SEQ ID NO: 201 - protein, 11 aa, Homo sapiens
ITDDQLLALLP

SEQ ID NO: 202 - protein, 11 aa, Homo sapiens
HGTLHLERLAY

SEQ ID NO: 203 - protein, 10 aa, Homo sapiens
GTLHLERLAY

SEQ ID NO: 204 - protein, 10 aa, Homo sapiens
CSQLTTLSFY

SEQ ID NO: 205 - protein, 10 aa, Homo sapiens
LSLQCLQALY

SEQ ID NO: 206 - protein, 9 aa, Homo sapiens
PTLAKFSPY

SEQ ID NO: 207 - protein, 9 aa, Homo sapiens
LSNLTHVLY

SEQ ID NO: 208 - protein, 9 aa, Homo sapiens
WSGNRASLY

SEQ ID NO: 209 - protein, 9 aa, Homo sapiens
LSHIHASSY

SEQ ID NO: 210 - protein, 10 aa, Homo sapiens
ALLERASATL

SEQ ID NO: 211 - protein, 10 aa, Homo sapiens
ALAIAALELL

SEQ ID NO: 212 - protein, 10 aa, Homo sapiens
SLSGVMLTDV

SEQ ID NO: 213 - protein, 10 aa, Homo sapiens
ALYVDSLFFL

SEQ ID NO: 214 - protein, 9 aa, Homo sapiens
QLLALLPSL

SEQ ID NO: 215 - protein, 9 aa, Homo sapiens
SLLQHLIGL

SEQ ID NO: 216 - protein, 9 aa, Homo sapiens
RLRELLCEL

SEQ ID NO: 217 - protein, 9 aa, Homo sapiens
YLHARLREL

SEQ ID NO: 218 - protein, 9 aa, Homo sapiens
ALAIAALEL

SEQ ID NO: 219 - protein, 9 aa, Homo sapiens
FLRGRLDQL

SEQ ID NO: 220 - protein, 10 aa, Homo sapiens
HLIGLSNLTH

SEQ ID NO: 221 - protein, 10 aa, Homo sapiens
RLWGSIQSRY

SEQ ID NO: 222 - protein, 10 aa, Homo sapiens
KVKRKKNVLR

SEQ ID NO: 223 - protein, 10 aa, Homo sapiens
VLYPVPLESY

SEQ ID NO: 224 - protein, 9 aa, Homo sapiens
CLPLGVLMK

SEQ ID NO: 225 - protein, 9 aa, Homo sapiens
ELAGQSLLK

SEQ ID NO: 226 - protein, 9 aa, Homo sapiens
KLQVLDLRK

SEQ ID NO: 227 - protein, 9 aa, Homo sapiens
RLSEGDVMH

SEQ ID NO: 228 - protein, 9 aa, Homo sapiens
YLIEKVKRK

SEQ ID NO: 229 - protein, 9 aa, Homo sapiens
NVLRLCCKK

SEQ ID NO: 230 - protein, 10 aa, Homo sapiens
KVKRKKNVLR

SEQ ID NO: 231 - protein, 10 aa, Homo sapiens
PMQDIKMILK

SEQ ID NO: 232 - protein, 10 aa, Homo sapiens
CTWKLPTLAK

SEQ ID NO: 233 - protein, 10 aa, Homo sapiens
AIAALELLPR

SEQ ID NO: 234 - protein, 10 aa, Homo sapiens
AVLDGLDVLL

SEQ ID NO: 235 - protein, 10 aa, Homo sapiens
FSYLIEKVKR

SEQ ID NO: 236 - protein, 9 aa, Homo sapiens
ELAGQSLLK

SEQ ID NO: 237 - protein, 9 aa, Homo sapiens
EVLVDLFLK

SEQ ID NO: 238 - protein, 9 aa, Homo sapiens
ASSYISPEK

SEQ ID NO: 239 - protein, 9 aa, Homo sapiens
ELFSYLIEK

SEQ ID NO: 240 - protein, 10 aa, Homo sapiens
QYIAQFTSQF

SEQ ID NO: 241 - protein, 10 aa, Homo sapiens
AYLHARLREL

SEQ ID NO: 242 - protein, 10 aa, Homo sapiens
LFPPLFMAAF

SEQ ID NO: 243 - protein, 10 aa, Homo sapiens
KFSPYLGQMI

SEQ ID NO: 244 - protein, 10 aa, Homo sapiens
FFLRGRLDQL

SEQ ID NO: 245 - protein, 10 aa, Homo sapiens
VSPEPLQALL

SEQ ID NO: 246 - protein, 9 aa, Homo sapiens
SYEDIHGTL

SEQ ID NO: 247 - protein, 9 aa, Homo sapiens
PYLGQMINL

SEQ ID NO: 248 - protein, 9 aa, Homo sapiens
LYVDSLFFL

SEQ ID NO: 249 - protein, 9 aa, Homo sapiens
TFYDPEPIL

SEQ ID NO: 250 - protein, 10 aa, Homo sapiens
ETFKAVLDGL

SEQ ID NO: 251 - protein, 10 aa, Homo sapiens
DVSPEPLQAL

SEQ ID NO: 252 - protein, 10 aa, Homo sapiens
ETLSITNCRL

SEQ ID NO: 253 - protein, 10 aa, Homo sapiens
EGACDELFSY

SEQ ID NO: 254 - protein, 10 aa, Homo sapiens
EKEEQYIAQF

SEQ ID NO: 255 - protein, 10 aa, Homo sapiens
SVSQLSVLSL

SEQ ID NO: 256 - protein, 9 aa, Homo sapiens
EVRPRRWKL

SEQ ID NO: 257 - protein, 9 aa, Homo sapiens
ETFKAVLDG

SEQ ID NO: 258 - protein, 9 aa, Homo sapiens
EVLVDLFLK

SEQ ID NO: 259 - protein, 11 aa, Homo sapiens
DVLLAQEVRPR

SEQ ID NO: 260 - protein, 10 aa, Homo sapiens
EAAQPMTKKR

SEQ ID NO: 261 - protein, 10 aa, Homo sapiens
KVKRKKNVLR

SEQ ID NO: 262 - protein, 9 aa, Homo sapiens
EAAQPMTKK

SEQ ID NO: 263 - protein, 9 aa, Homo sapiens
EVLVDLFLK

SEQ ID NO: 264 - protein, 9 aa, Homo sapiens
ELFSYLIEK

SEQ ID NO: 265 - protein, 9 aa, Homo sapiens
ETLSITNCR

SEQ ID NO: 266 - protein, 9 aa, Homo sapiens
DVLLAQEVR

SEQ ID NO: 267 - protein, 9 aa, Homo sapiens
DSLFFLRGR

SEQ ID NO: 268 - protein, 9 aa, Homo sapiens
IAALELLPR

SEQ ID NO: 269 - protein, 10 aa, Homo sapiens
RPRRWKLQVL

SEQ ID NO: 270 - protein, 10 aa, Homo sapiens
SPSVSQLSVL

SEQ ID NO: 271 - protein, 10 aa, Homo sapiens
LPSLSHCSQL

SEQ ID NO: 272 - protein, 10 aa, Homo sapiens
MPMQDIKMIL

SEQ ID NO: 273 - protein, 9 aa, Homo sapiens
LPRELFPPL

SEQ ID NO: 274 - protein, 9 aa, Homo sapiens
QPFIPVEVL

SEQ ID NO: 275 - protein, 9 aa, Homo sapiens
IPVEVLVDL

SEQ ID NO: 276 - protein, 9 aa, Homo sapiens
SPEPLQALL

SEQ ID NO: 277 - protein, 9 aa, Homo sapiens
RPRRWKLQV

SEQ ID NO: 278 - protein, 9 aa, Homo sapiens
RPSMVWLSA

SEQ ID NO: 279 - protein, 9 aa, Homo sapiens
TKKRKVDGL

SEQ ID NO: 280 - protein, 9 aa, Homo sapiens
FLRGRLDQL

SEQ ID NO: 281 - protein, 9 aa, Homo sapiens
KVKRKKNVL

SEQ ID NO: 282 - protein, 9 aa, Homo sapiens
EVRPRRWKL

SEQ ID NO: 283 - protein, 9 aa, Homo sapiens
PRRWKLQVL

SEQ ID NO: 284 - protein, 9 aa, Homo sapiens
VLRLCCKKL

SEQ ID NO: 285 - protein, 9 aa, Homo sapiens
YLHARLREL

SEQ ID NO: 286 - protein, 9 aa, Homo sapiens
RLRELLCEL

SEQ ID NO: 287 - protein, 8 aa, Homo sapiens
HARLRELL

SEQ ID NO: 288 - protein, 8 aa, Homo sapiens
VKRKKNVL

SEQ ID NO: 289 - protein, 10 aa, Homo sapiens
VLYPVPLESY

SEQ ID NO: 290 - protein, 10 aa, Homo sapiens
RLWGSIQSRY

SEQ ID NO: 291 - protein, 10 aa, Homo sapiens
GLSNLTHVLY

SEQ ID NO: 292 - protein, 10 aa, Homo sapiens
RLCCKKLKIF

SEQ ID NO: 293 - protein, 10 aa, Homo sapiens
LLSHIHASSY

SEQ ID NO: 294 - protein, 9 aa, Homo sapiens
TLHLERLAY

SEQ ID NO: 295 - protein, 9 aa, Homo sapiens
GQHLHLETF

SEQ ID NO: 296 - protein, 9 aa, Homo sapiens
SLQCLQALY

SEQ ID NO: 297 - protein, 9 aa, Homo sapiens
ALYVDSLFF

SEQ ID NO: 298 - protein, 9 aa, Homo sapiens
SQLTTLSFY

SEQ ID NO: 299 - protein, 9 aa, Homo sapiens
DEALAIAAL

SEQ ID NO: 300 - protein, 9 aa, Homo sapiens
LELLPRELF

SEQ ID NO: 301 - protein, 9 aa, Homo sapiens
KEGACDELF

SEQ ID NO: 302 - protein, 9 aa, Homo sapiens
PEPILCPCF

SEQ ID NO: 303 - protein, 8 aa, Homo sapiens
VEVLVDLF

SEQ ID NO: 304 - protein, 8 aa, Homo sapiens
EEQYIAQF

SEQ ID NO: 305 - protein, 8 aa, Homo sapiens
LELLPREL

SEQ ID NO: 306 - protein, 8 aa, Homo sapiens
RELFPPLF

SEQ ID NO: 307 - protein, 8 aa, Homo sapiens
SEGDVMHL

SEQ ID NO: 308 - protein, 8 aa, Homo sapiens
LERASATL

SEQ ID NO: 309 - protein, 10 aa, Homo sapiens
RRLWGSIQSR

SEQ ID NO: 310 - protein, 10 aa, Homo sapiens
RRWKLQVLDL

SEQ ID NO: 311 - protein, 10 aa, Homo sapiens
ERLAYLHARL

SEQ ID NO: 312 - protein, 10 aa, Homo sapiens
ARLRELLCEL

SEQ ID NO: 313 - protein, 9 aa, Homo sapiens
KRKKNVLRL

SEQ ID NO: 314 - protein, 9 aa, Homo sapiens
RRLLLSHIH

SEQ ID NO: 315 - protein, 9 aa, Homo sapiens
GRLDQLLRH

SEQ ID NO: 316 - protein, 9 aa, Homo sapiens
PRRWKLQVL

SEQ ID NO: 317 - protein, 9 aa, Homo sapiens
LRLCCKKLK

SEQ ID NO: 318 - protein, 9 aa, Homo sapiens
ERLAYLHAR

SEQ ID NO: 319 - protein, 10 aa, Homo sapiens
RPRRWKLQVL

SEQ ID NO: 320 - protein, 10 aa, Homo sapiens
SPSVSQLSVL

SEQ ID NO: 321 - protein, 10 aa, Homo sapiens
LPRELFPPLF

SEQ ID NO: 322 - protein, 10 aa, Homo sapiens
IPVEVLVDLF

SEQ ID NO: 323 - protein, 10 aa, Homo sapiens
MPMQDIKMIL

SEQ ID NO: 324 - protein, 10 aa, Homo sapiens
LPTLAKFSPY

SEQ ID NO: 325 - protein, 9 aa, Homo sapiens
IPVEVLVDL

SEQ ID NO: 326 - protein, 9 aa, Homo sapiens
LPRELFPPL

SEQ ID NO: 327 - protein, 9 aa, Homo sapiens
SPEPLQALL

SEQ ID NO: 328 - protein, 9 aa, Homo sapiens
QPFIPVEVL

SEQ ID NO: 329 - protein, 10 aa, Homo sapiens
MSVWTSPRRL

SEQ ID NO: 330 - protein, 10 aa, Homo sapiens
AALELLPREL

SEQ ID NO: 331 - protein, 10 aa, Homo sapiens
KAVLDGLDVL

SEQ ID NO: 332 - protein, 10 aa, Homo sapiens
LAQEVRPRRW

SEQ ID NO: 333 - protein, 10 aa, Homo sapiens
ESYEDIHGTL

SEQ ID NO: 334 - protein, 10 aa, Homo sapiens
LSLQCLQALY

SEQ ID NO: 335 - protein, 10 aa, Homo sapiens
VSPEPLQALL

SEQ ID NO: 336 - protein, 10 aa, Homo sapiens
LSHCSQLTTL

SEQ ID NO: 337 - protein, 9 aa, Homo sapiens
KAMVQAWPF

SEQ ID NO: 338 - protein, 9 aa, Homo sapiens
KVKRKKNVL

SEQ ID NO: 339 - protein, 15 aa, Homo sapiens
PRRLVELAGQSLLKD

SEQ ID NO: 340 - protein, 15 aa, Homo sapiens
LDGLDVLLAQEVRPR

SEQ ID NO: 341 - protein, 15 aa, Homo sapiens
FLSLQCLQALYVDSL

SEQ ID NO: 342 - protein, 15 aa, Homo sapiens
RHVMNPLETLSITNC

SEQ ID NO: 343 - protein, 15 aa, Homo sapiens
QLSVLSLSGVMLTDV

SEQ ID NO: 344 - protein, 15 aa, Homo sapiens
RRLWGSIQSRYISMS

SEQ ID NO: 345 - protein, 15 aa, Homo sapiens
EEQYIAQFTSQFLSL

SEQ ID NO: 346 - protein, 15 aa, Homo sapiens
DDQLLALLPSLSHCS

SEQ ID NO: 347 - protein, 15 aa, Homo sapiens
GVMLTDVSPEPLQAL

SEQ ID NO: 348 - protein, 15 aa, Homo sapiens
GQSLLKDEALAIAAL

SEQ ID NO: 349 - protein, 15 aa, Homo sapiens
ECGITDDQLLALLPS

SEQ ID NO: 350 - protein, 15 aa, Homo sapiens
LKMVQLDSIEDLEVT

SEQ ID NO: 351 - protein, 15 aa, Homo sapiens
LQALYVDSLFFLRGR

SEQ ID NO: 352 - protein, 15 aa, Homo sapiens
RRLVELAGQSLLKDE

SEQ ID NO: 353 - protein, 15 aa, Homo sapiens
IAALELLPRELFPPL

SEQ ID NO: 354 - protein, 15 aa, Homo sapiens
LGQMINLRRLLLSHI

SEQ ID NO: 355 - protein, 15 aa, Homo sapiens
FWTVWSGNRASLYSF

SEQ ID NO: 356 - protein, 15 aa, Homo sapiens
SSYISPEKEEQYIAQ

SEQ ID NO: 357 - protein, 15 aa, Homo sapiens
LAYLHARLRELLCEL

SEQ ID NO: 358 - protein, 15 aa, Homo sapiens
GQSLLKDEALAIAAL

SEQ ID NO: 359 - protein, 15 aa, Homo sapiens
RRLWGSIQSRYISMS

SEQ ID NO: 360 - protein, 15 aa, Homo sapiens
RRLVELAGQSLLKDE

SEQ ID NO: 361 - protein, 15 aa, Homo sapiens
SYLIEKVKRKKNVLR

SEQ ID NO: 362 - protein, 15 aa, Homo sapiens
LGQMINLRRLLLSHI

SEQ ID NO: 363 - protein, 15 aa, Homo sapiens
EQYIAQFTSQFLSLQ

SEQ ID NO: 364 - protein, 15 aa, Homo sapiens
RGRLDQLLRHVMNPL

SEQ ID NO: 365 - protein, 15 aa, Homo sapiens
RHVMNPLETLSITNC

SEQ ID NO: 366 - protein, 15 aa, Homo sapiens
EGDVMHLSQSPSVSQ

SEQ ID NO: 367 - protein, 15 aa, Homo sapiens
LALLPSLSHCSQLTT

SEQ ID NO: 368 - protein, 15 aa, Homo sapiens
SISISALQSLLQHLI

SEQ ID NO: 369 - protein, 15 aa, Homo sapiens
RRLWGSIQSRYISMS

SEQ ID NO: 370 - protein, 15 aa, Homo sapiens
IEDLEVTCTWKLPTL

SEQ ID NO: 371 - protein, 15 aa, Homo sapiens
GDVMHLSQSPSVSQL

SEQ ID NO: 372 - protein, 15 aa, Homo sapiens
MVQLDSIEDLEVTCT

SEQ ID NO: 373 - protein, 15 aa, Homo sapiens
LSFYGNSISISALQS

SEQ ID NO: 374 - protein, 15 aa, Homo sapiens
MAAFDGRHSQTLKAM

SEQ ID NO: 375 - protein, 15 aa, Homo sapiens
EEQYIAQFTSQFLSL

SEQ ID NO: 376 - protein, 15 aa, Homo sapiens
EQYIAQFTSQFLSLQ

SEQ ID NO: 377 - protein, 15 aa, Homo sapiens
RHVMNPLETLSITNC

SEQ ID NO: 378 - protein, 15 aa, Homo sapiens
LQALLERASATLQDL

SEQ ID NO: 379 - protein, 15 aa, Homo sapiens
TWKLPTLAKFSPYLG

SEQ ID NO: 380 - protein, 15 aa, Homo sapiens
QSRYISMSVWTSPRR

SEQ ID NO: 381 - protein, 15 aa, Homo sapiens
AQPMTKKRKVDGLST

SEQ ID NO: 382 - protein, 15 aa, Homo sapiens
TSQFLSLQCLQALYV

SEQ ID NO: 383 - protein, 15 aa, Homo sapiens
MSVWTSPRRLVELAG

SEQ ID NO: 384 - protein, 15 aa, Homo sapiens
IAALELLPRELFPPL

SEQ ID NO: 385 - protein, 15 aa, Homo sapiens
CLPLGVLMKGQHLHL

SEQ ID NO: 386 - protein, 15 aa, Homo sapiens
QDFWTVWSGNRASLY

SEQ ID NO: 387 - protein, 15 aa, Homo sapiens
SYLIEKVKRKKNVLR

SEQ ID NO: 388 - protein, 15 aa, Homo sapiens
MQDIKMILKMVQLDS

SEQ ID NO: 389 - protein, 15 aa, Homo sapiens
HLHLETFKAVLDGLD

SEQ ID NO: 390 - protein, 15 aa, Homo sapiens
PVPLESYEDIHGTLH

SEQ ID NO: 391 - protein, 15 aa, Homo sapiens
YISMSVWTSPRRLVE

SEQ ID NO: 392 - protein, 15 aa, Homo sapiens
PLFMAAFDGRHSQTL

SEQ ID NO: 393 - protein, 15 aa, Homo sapiens
LPTLAKFSPYLGQMI

SEQ ID NO: 394 - protein, 15 aa, Homo sapiens
EQYIAQFTSQFLSLQ

SEQ ID NO: 395 - protein, 15 aa, Homo sapiens
LTTLSFYGNSISISA

SEQ ID NO: 396 - protein, 15 aa, Homo sapiens
LAKFSPYLGQMINLR

SEQ ID NO: 397 - protein, 15 aa, Homo sapiens
MERRRLWGSIQSRYI

SEQ ID NO: 398 - protein, 15 aa, Homo sapiens
GSIQSRYISMSVWTS

SEQ ID NO: 399 - protein, 141 aa, Homo sapiens
MGAPTLPPAWQPFLKDHRISTFKNWPFLEGCACTPERMAEAGFIHCPTENEPDLQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 400 - protein, 11 aa, Homo sapiens
PTENEPDLAQC

SEQ ID NO: 401 - protein, 11 aa, Homo sapiens
KLDRERAKNKI

SEQ ID NO: 402 - protein, 11 aa, Homo sapiens
LKDHRISTFKN

SEQ ID NO: 403 - protein, 11 aa, Homo sapiens
STFKNWPFLEG

SEQ ID NO: 404 - protein, 11 aa, Homo sapiens
DDDPIEEHKKH

SEQ ID NO: 405 - protein, 10 aa, Homo sapiens
PTENEPDLAQ

SEQ ID NO: 406 - protein, 9 aa, Homo sapiens
PTENEPDLA

SEQ ID NO: 407 - protein, 9 aa, Homo sapiens
LTLGEFLKL

SEQ ID NO: 408 - protein, 9 aa, Homo sapiens
LGEFLKLDR

SEQ ID NO: 409 - protein, 9 aa, Homo sapiens
KLDRERAKN

SEQ ID NO: 410 - protein, 10 aa, Homo sapiens
TLPPAWQPFL

SEQ ID NO: 411 - protein, 10 aa, Homo sapiens
ELTLGEFLKL

SEQ ID NO: 412 - protein, 10 aa, Homo sapiens
FLKDHRISTF

SEQ ID NO: 413 - protein, 9 aa, Homo sapiens
LTLGEFLKL

SEQ ID NO: 414 - protein, 9 aa, Homo sapiens
KVRRAIEQL

SEQ ID NO: 415 - protein, 9 aa, Homo sapiens
RAIEQLAAM

SEQ ID NO: 416 - protein, 9 aa, Homo sapiens
STFKNWPFL

SEQ ID NO: 417 - protein, 9 aa, Homo sapiens
FLKDHRIST

SEQ ID NO: 418 - protein, 9 aa, Homo sapiens
SVKKQFEEL

SEQ ID NO: 419 - protein, 9 aa, Homo sapiens
TLGEFLKLD

SEQ ID NO: 420 - protein, 10 aa, Homo sapiens
KLDRERAKNK

SEQ ID NO: 421 - protein, 10 aa, Homo sapiens
FLKDHRISTF

SEQ ID NO: 422 - protein, 10 aa, Homo sapiens
FLKLDRERAK

SEQ ID NO: 423 - protein, 10 aa, Homo sapiens
KIAKETNNKK

SEQ ID NO: 424 - protein, 10 aa, Homo sapiens
DLAQCFFCFK

SEQ ID NO: 425 - protein, 9 aa, Homo sapiens
ELTLGEFLK

SEQ ID NO: 426 - protein, 9 aa, Homo sapiens
KIAKETNNK

SEQ ID NO: 427 - protein, 9 aa, Homo sapiens
KVRRAIEQL

SEQ ID NO: 428 - protein, 9 aa, Homo sapiens
SGCAFLSVK

SEQ ID NO: 429 - protein, 9 aa, Homo sapiens
KLDRERAKN

SEQ ID NO: 430 - protein, 10 aa, Homo sapiens
SSGCAFLSVK

SEQ ID NO: 431 - protein, 10 aa, Homo sapiens
DLAQCFFCFK

SEQ ID NO: 432 - protein, 10 aa, Homo sapiens
SGCAFLSVKK

SEQ ID NO: 433 - protein, 10 aa, Homo sapiens
TLGEFLKLDR

SEQ ID NO: 434 - protein, 10 aa, Homo sapiens
STFKNWPFLE

SEQ ID NO: 435 - protein, 10 aa, Homo sapiens
KLDRERAKNK

SEQ ID NO: 436 - protein, 10 aa, Homo sapiens
KIAKETNNKK

SEQ ID NO: 437 - protein, 9 aa, Homo sapiens
SSGCAFLSV

SEQ ID NO: 438 - protein, 9 aa, Homo sapiens
GCAFLSVKK

SEQ ID NO: 439 - protein, 9 aa, Homo sapiens
ELTLGEFLK

SEQ ID NO: 440 - protein, 10 aa, Homo sapiens
QFEELTLGEF

SEQ ID NO: 441 - protein, 10 aa, Homo sapiens
TLPPAWQPFL

SEQ ID NO: 442 - protein, 10 aa, Homo sapiens
PDLAQCFFCF

SEQ ID NO: 443 - protein, 10 aa, Homo sapiens
PTLPPAWQPF

SEQ ID NO: 444 - protein, 10 aa, Homo sapiens
NEPDLAQCFF

SEQ ID NO: 445 - protein, 10 aa, Homo sapiens
LSVKKQFEEL

SEQ ID NO: 446 - protein, 10 aa, Homo sapiens
ELTLGEFLKL

SEQ ID NO: 447 - protein, 9 aa, Homo sapiens
AFLSVKKQF

SEQ ID NO: 448 - protein, 9 aa, Homo sapiens
LTLGEFLKL

SEQ ID NO: 449 - protein, 9 aa, Homo sapiens
TLPPAWQPF

SEQ ID NO: 450 - protein, 10 aa, Homo sapiens
ELTLGEFLKL

SEQ ID NO: 451 - protein, 10 aa, Homo sapiens
ENEPDLAQCF

SEQ ID NO: 452 - protein, 10 aa, Homo sapiens
ETAKKVRRAI

SEQ ID NO: 453 - protein, 10 aa, Homo sapiens
ETNNKKKEFE

SEQ ID NO: 454 - protein, 9 aa, Homo sapiens
ETNNKKKEF

SEQ ID NO: 455 - protein, 9 aa, Homo sapiens
ETAKKVRRA

SEQ ID NO: 456 - protein, 9 aa, Homo sapiens
KVRRAIEQL

SEQ ID NO: 457 - protein, 9 aa, Homo sapiens
STFKNWPFL

SEQ ID NO: 458 - protein, 9 aa, Homo sapiens
EELTLGEFL

SEQ ID NO: 459 - protein, 9 aa, Homo sapiens
SVKKQFEEL

SEQ ID NO: 460 - protein, 11 aa, Homo sapiens
LTLGEFLKLDR

SEQ ID NO: 461 - protein, 11 aa, Homo sapiens
PAWQPFLKDHR

SEQ ID NO: 462 - protein, 11 aa, Homo sapiens
SSGCAFLSVKK

SEQ ID NO: 463 - protein, 11 aa, Homo sapiens
EFEETAKKVRR

SEQ ID NO: 464 - protein, 11 aa, Homo sapiens
ETAKKVRRAIE

SEQ ID NO: 465 - protein, 10 aa, Homo sapiens
DLAQCFFCFK

SEQ ID NO: 466 - protein, 9 aa, Homo sapiens
EETAKKVRR

SEQ ID NO: 467 - protein, 9 aa, Homo sapiens
ERAKNKIAK

SEQ ID NO: 468 - protein, 9 aa, Homo sapiens
ETAKKVRRA

SEQ ID NO: 469 - protein, 9 aa, Homo sapiens
ELTLGEFLK

SEQ ID NO: 470 - protein, 9 aa, Homo sapiens
LPPAWQPFL

SEQ ID NO: 471 - protein, 9 aa, Homo sapiens
CPTENEPDL

SEQ ID NO: 472 - protein, 9 aa, Homo sapiens
EPDLAQCFF

SEQ ID NO: 473 - protein, 9 aa, Homo sapiens
APTLPPAWQ

SEQ ID NO: 474 - protein, 9 aa, Homo sapiens
QPFLKDHRI

SEQ ID NO: 475 - protein, 9 aa, Homo sapiens
KHSSGCAFL

SEQ ID NO: 476 - protein, 9 aa, Homo sapiens
LTLGEFLKL

SEQ ID NO: 477 - protein, 10 aa, Homo sapiens
WPFLEGCACT

SEQ ID NO: 478 - protein, 10 aa, Homo sapiens
TPERMAEAGF

SEQ ID NO: 479 - protein, 10 aa, Homo sapiens
CPTENEPDLA

SEQ ID NO: 480 - protein, 9 aa, Homo sapiens
RAKNKIAKE

SEQ ID NO: 481 - protein, 9 aa, Homo sapiens
QPFLKDHRI

SEQ ID NO: 482 - protein, 9 aa, Homo sapiens
SVKKQFEEL

SEQ ID NO: 483 - protein, 9 aa, Homo sapiens
NNKKKEFEE

SEQ ID NO: 484 - protein, 9 aa, Homo sapiens
TAKKVRRAI

SEQ ID NO: 485 - protein, 8 aa, Homo sapiens
AKKVRRAI

SEQ ID NO: 486 - protein, 8 aa, Homo sapiens
FLSVKKQF

SEQ ID NO: 487 - protein, 8 aa, Homo sapiens
RAKNKIAK

SEQ ID NO: 488 - protein, 8 aa, Homo sapiens
RERAKNKI

SEQ ID NO: 489 - protein, 8 aa, Homo sapiens
VKKQFEEL

SEQ ID NO: 490 - protein, 10 aa, Homo sapiens
FLKDHRISTF

SEQ ID NO: 491 - protein, 10 aa, Homo sapiens
KQFEELTLGE

SEQ ID NO: 492 - protein, 10 aa, Homo sapiens
TLPPAWQPFL

SEQ ID NO: 493 - protein, 10 aa, Homo sapiens
ELEGWEPDDD

SEQ ID NO: 494 - skipped

SEQ ID NO: 495 - protein, 10 aa, Homo sapiens
TLGEFLKLDR

SEQ ID NO: 496 - protein, 9 aa, Homo sapiens
TLPPAWQPF

SEQ ID NO: 497 - protein, 9 aa, Homo sapiens
DLAQCFFCF

SEQ ID NO: 498 - protein, 9 aa, Homo sapiens
KQFEELTLG

SEQ ID NO: 499 - protein, 9 aa, Homo sapiens
FLKDHRIST

SEQ ID NO: 500 - protein, 9 aa, Homo sapiens
KVRRAIEQL

SEQ ID NO: 501 - protein, 9 aa, Homo sapiens
EELTLGEFL

SEQ ID NO: 502 - protein, 9 aa, Homo sapiens
FEELTLGEF

SEQ ID NO: 503 - protein, 9 aa, Homo sapiens
NEPDLAQCF

SEQ ID NO: 504 - protein, 9 aa, Homo sapiens
PERMAEAGF

SEQ ID NO: 505 - protein, 9 aa, Homo sapiens
DLAQCFFCF

SEQ ID NO: 506 - protein, 9 aa, Homo sapiens
KELEGWEPD

SEQ ID NO: 507 - protein, 8 aa, Homo sapiens
EELTLGEF

SEQ ID NO: 508 - protein, 8 aa, Homo sapiens
EEHKKHSS

SEQ ID NO: 509 - protein, 8 aa, Homo sapiens
KELEGWEP

SEQ ID NO: 510 - protein, 8 aa, Homo sapiens
KQFEELTL

SEQ ID NO: 511 - protein, 10 aa, Homo sapiens
RRAIEQLAAM

SEQ ID NO: 512 - protein, 10 aa, Homo sapiens
GEFLKLDRER

SEQ ID NO: 513 - protein, 10 aa, Homo sapiens
ERMAEAGFIH

SEQ ID NO: 514 - protein, 10 aa, Homo sapiens
ERAKNKIAKE

SEQ ID NO: 515 - protein, 10 aa, Homo sapiens
KIAKETNNKK

SEQ ID NO: 516 - protein, 9 aa, Homo sapiens
ERAKNKIAK

SEQ ID NO: 517 - protein, 9 aa, Homo sapiens
DRERAKNKI

SEQ ID NO: 518 - protein, 9 aa, Homo sapiens
KEFEETAKK

SEQ ID NO: 519 - protein, 9 aa, Homo sapiens
ERMAEAGFI

SEQ ID NO: 520 - protein, 9 aa, Homo sapiens
GCAFLSVKK

SEQ ID NO: 521 - protein, 10 aa, Homo sapiens
TPERMAEAGF

SEQ ID NO: 522 - protein, 10 aa, Homo sapiens
LPPAWQPFLK

SEQ ID NO: 523 - protein, 10 aa, Homo sapiens
EPDDDPIEEH

SEQ ID NO: 524 - protein, 10 aa, Homo sapiens
LSVKKQFEEL

SEQ ID NO: 525 - protein, 9 aa, Homo sapiens
LPPAWQPFL

SEQ ID NO: 526 - protein, 9 aa, Homo sapiens
CPTENEPDL

SEQ ID NO: 527 - protein, 9 aa, Homo sapiens
EPDLAQCFF

SEQ ID NO: 528 - protein, 9 aa, Homo sapiens
QPFLKDHRI

SEQ ID NO: 529 - protein, 9 aa, Homo sapiens
TPERMAEAG

SEQ ID NO: 530 - protein, 9 aa, Homo sapiens
EPDDDPIEE

SEQ ID NO: 531 - protein, 10 aa, Homo sapiens
ETAKKVRRAI

SEQ ID NO: 532 - protein, 10 aa, Homo sapiens
PTLPPAWQPF

SEQ ID NO: 533 - protein, 10 aa, Homo sapiens
ISTFKNWPFL

SEQ ID NO: 534 - protein, 10 aa, Homo sapiens
LSVKKQFEEL

SEQ ID NO: 535 - protein, 9 aa, Homo sapiens
TAKKVRRAI

SEQ ID NO: 536 - protein, 9 aa, Homo sapiens
RAIEQLAAM

SEQ ID NO: 537 - protein, 9 aa, Homo sapiens
KVRRAIEQL

SEQ ID NO: 538 - protein, 9 aa, Homo sapiens
ISTFKNWPF

SEQ ID NO: 539 - protein, 9 aa, Homo sapiens
LTLGEFLKL

SEQ ID NO: 540 - protein, 9 aa, Homo sapiens
GAPTLPPAW

SEQ ID NO: 541 - protein, 15 aa, Homo sapiens
FFCFKELEGWEPDDD

SEQ ID NO: 542 - protein, 15 aa, Homo sapiens
FKNWPFLEGCACTPE

SEQ ID NO: 543 - protein, 15 aa, Homo sapiens
LGEFLKLDRERAKNK

SEQ ID NO: 544 - protein, 15 aa, Homo sapiens
NWPFLEGCACTPERM

SEQ ID NO: 545 - protein, 15 aa, Homo sapiens
KKQFEELTLGEFLKL

SEQ ID NO: 546 - protein, 15 aa, Homo sapiens
CTPERMAEAGFIHCP

SEQ ID NO: 547 - protein, 15 aa, Homo sapiens
FEELTLGEFLKLDRE

SEQ ID NO: 548 - protein, 15 aa, Homo sapiens
MGAPTLPPAWQPFLK

SEQ ID NO: 549 - protein, 15 aa, Homo sapiens
KKKEFEETAKKVRRA

SEQ ID NO: 550 - protein, 15 aa, Homo sapiens
AKKVRRAIEQLAAMD

SEQ ID NO: 551 - protein, 15 aa, Homo sapiens
GEFLKLDRERAKNKI

SEQ ID NO: 552 - protein, 15 aa, Homo sapiens
WQPFLKDHRISTFKN

SEQ ID NO: 553 - protein, 15 aa, Homo sapiens
APTLPPAWQPFLKDH

SEQ ID NO: 554 - protein, 15 aa, Homo sapiens
DHRISTFKNWPFLEG

SEQ ID NO: 555 - protein, 15 aa, Homo sapiens
FEELTLGEFLKLDRE

SEQ ID NO: 556 - protein, 15 aa, Homo sapiens
PTENEPDLAQCFFCF

SEQ ID NO: 557 - protein, 15 aa, Homo sapiens
QPFLKDHRISTFKNW

SEQ ID NO: 558 - protein, 15 aa, Homo sapiens
GCAFLSVKKQFEELT

SEQ ID NO: 559 - protein, 15 aa, Homo sapiens
ELTLGEFLKLDRERA

SEQ ID NO: 560 - protein, 15 aa, Homo sapiens
AKKVRRAIEQLAAMD

SEQ ID NO: 561 - protein, 15 aa, Homo sapiens
WQPFLKDHRISTFKN

SEQ ID NO: 562 - protein, 15 aa, Homo sapiens
LGEFLKLDRERAKNK

SEQ ID NO: 563 - protein, 15 aa, Homo sapiens
APTLPPAWQPFLKDH

SEQ ID NO: 564 - protein, 15 aa, Homo sapiens
KNKIAKETNNKKKEF

SEQ ID NO: 565 - protein, 15 aa, Homo sapiens
DHRISTFKNWPFLEG

SEQ ID NO: 566 - protein, 15 aa, Homo sapiens
GEFLKLDRERAKNKI

SEQ ID NO: 567 - protein, 15 aa, Homo sapiens
FLKLDRERAKNKIAK

SEQ ID NO: 568 - protein, 15 aa, Homo sapiens
AKKVRRAIEQLAAMD

SEQ ID NO: 569 - protein, 15 aa, Homo sapiens
FLKDHRISTFKNWPF

SEQ ID NO: 570 - protein, 15 aa, Homo sapiens
RMAEAGFIHCPTENE

SEQ ID NO: 571 - protein, 15 aa, Homo sapiens
AKKVRRAIEQLAAMD

SEQ ID NO: 572 - protein, 15 aa, Homo sapiens
APTLPPAWQPFLKDH

SEQ ID NO: 573 - protein, 15 aa, Homo sapiens
DHRISTFKNWPFLEG

SEQ ID NO: 574 - protein, 15 aa, Homo sapiens
LEGCACTPERMAEAG

SEQ ID NO: 575 - protein, 15 aa, Homo sapiens
EAGFIHCPTENEPDL

SEQ ID NO: 576 - protein, 15 aa, Homo sapiens
KKEFEETAKKVRRAI

SEQ ID NO: 577 - protein, 15 aa, Homo sapiens
AQCFFCFKELEGWEP

SEQ ID NO: 578 - protein, 15 aa, Homo sapiens
QCFFCFKELEGWEPD

SEQ ID NO: 579 - protein, 15 aa, Homo sapiens
LEGWEPDDDPIEEHK

SEQ ID NO: 580 - protein, 15 aa, Homo sapiens
KKQFEELTLGEFLKL

SEQ ID NO: 581 - protein, 15 aa, Homo sapiens
LGEFLKLDRERAKNK

SEQ ID NO: 582 - protein, 15 aa, Homo sapiens
GCAFLSVKKQFEELT

SEQ ID NO: 583 - protein, 15 aa, Homo sapiens
FFCFKELEGWEPDDD

SEQ ID NO: 584 - protein, 15 aa, Homo sapiens
DDPIEEHKKHSSGCA

SEQ ID NO: 585 - protein, 15 aa, Homo sapiens
KKEFEETAKKVRRAI

SEQ ID NO: 586 - protein, 15 aa, Homo sapiens
PPAWQPFLKDHRIST

SEQ ID NO: 587 - protein, 15 aa, Homo sapiens
WQPFLKDHRISTFKN

SEQ ID NO: 588 - protein, 15 aa, Homo sapiens
AWQPFLKDHRISTFK

SEQ ID NO: 589 - protein, 15 aa, Homo sapiens
AQCFFCFKELEGWEP

SEQ ID NO: 590 - protein, 15 aa, Homo sapiens
ISTFKNWPFLEGCAC

SEQ ID NO: 591 - protein, 15 aa, Homo sapiens
LGEFLKLDRERAKNK

SEQ ID NO: 592 - protein, 15 aa, Homo sapiens
GCAFLSVKKQFEELT

SEQ ID NO: 593 - protein, 15 aa, Homo sapiens
FFCFKELEGWEPDDD

SEQ ID NO: 594 - protein, 15 aa, Homo sapiens
DDPIEEHKKHSSGCA

SEQ ID NO: 595 - protein, 15 aa, Homo sapiens
KKEFEETAKKVRRAI

SEQ ID NO: 596 - protein, 15 aa, Homo sapiens
PPAWQPFLKDHRIST

SEQ ID NO: 597 - protein, 15 aa, Homo sapiens
WQPFLKDHRISTFKN

SEQ ID NO: 598 - protein, 15 aa, Homo sapiens
AWQPFLKDHRISTFK

SEQ ID NO: 599 - protein, 15 aa, Homo sapiens
AQCFFCFKELEGWEP

SEQ ID NO: 600 - protein, 15 aa, Homo sapiens
ISTFKNWPFLEGCAC

SEQ ID NO: 601 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 602 - protein, 11 aa, Homo sapiens
RGPESRLLEFY

SEQ ID NO: 603 - protein, 11 aa, Homo sapiens
AADHRQLQLSI

SEQ ID NO: 604 - protein, 11 aa, Homo sapiens
EAELARRSLAQ

SEQ ID NO: 605 - protein, 10 aa, Homo sapiens
GPESRLLEFY

SEQ ID NO: 606 - protein, 10 aa, Homo sapiens
AQDAPPLPVP

SEQ ID NO: 607 - protein, 10 aa, Homo sapiens
AADHRQLQLS

SEQ ID NO: 608 - protein, 10 aa, Homo sapiens
EAELARRSLA

SEQ ID NO: 609 - protein, 9 aa, Homo sapiens
PESRLLEFY

SEQ ID NO: 610 - protein, 9 aa, Homo sapiens
AQDAPPLPV

SEQ ID NO: 611 - protein, 9 aa, Homo sapiens
AADHRQLQL

SEQ ID NO: 612 - protein, 10 aa, Homo sapiens
LLMWITQCFL

SEQ ID NO: 613 - protein, 10 aa, Homo sapiens
DAPPLPVPGV

SEQ ID NO: 614 - protein, 10 aa, Homo sapiens
RLLEFYLAMP

SEQ ID NO: 615 - protein, 10 aa, Homo sapiens
FTVSGNILTI

SEQ ID NO: 616 - protein, 10 aa, Homo sapiens
QLQLSISSCL

SEQ ID NO: 617 - protein, 9 aa, Homo sapiens
SLAQDAPPL

SEQ ID NO: 618 - protein, 9 aa, Homo sapiens
SISSCLQQL

SEQ ID NO: 619 - protein, 9 aa, Homo sapiens
RLLEFYLAM

SEQ ID NO: 620 - protein, 9 aa, Homo sapiens
TVSGNILTI

SEQ ID NO: 621 - protein, 9 aa, Homo sapiens
LMWITQCFL

SEQ ID NO: 622 - protein, 10 aa, Homo sapiens
PLPVPGVLLK

SEQ ID NO: 623 - protein, 10 aa, Homo sapiens
RLLEFYLAMP

SEQ ID NO: 624 - protein, 10 aa, Homo sapiens
ELARRSLAQD

SEQ ID NO: 625 - protein, 10 aa, Homo sapiens
TIRLTAADHR

SEQ ID NO: 626 - protein, 10 aa, Homo sapiens
RLTAADHRQL

SEQ ID NO: 627 - protein, 10 aa, Homo sapiens
QLSISSCLQQ

SEQ ID NO: 628 - protein, 10 aa, Homo sapiens
FLAQPPSGQR

SEQ ID NO: 629 - protein, 9 aa, Homo sapiens
TIRLTAADH

SEQ ID NO: 630 - protein, 9 aa, Homo sapiens
RLLEFYLAM

SEQ ID NO: 631 - protein, 9 aa, Homo sapiens
ELARRSLAQ

SEQ ID NO: 632 - protein, 10 aa, Homo sapiens
ATPMEAELAR

SEQ ID NO: 633 - protein, 10 aa, Homo sapiens
PLPVPGVLLK

SEQ ID NO: 634 - protein, 10 aa, Homo sapiens
ASGPGGGAPR

SEQ ID NO: 635 - protein, 10 aa, Homo sapiens
TVSGNILTIR

SEQ ID NO: 636 - protein, 10 aa, Homo sapiens
GVLLKEFTVS

SEQ ID NO: 637 - protein, 9 aa, Homo sapiens
ASGLNGCCR

SEQ ID NO: 638 - protein, 9 aa, Homo sapiens
LPVPGVLLK

SEQ ID NO: 639 - protein, 9 aa, Homo sapiens
VSGNILTIR

SEQ ID NO: 640 - protein, 9 aa, Homo sapiens
FTVSGNILT

SEQ ID NO: 641 - protein, 9 aa, Homo sapiens
SSCLQQLSL

SEQ ID NO: 642 - protein, 10 aa, Homo sapiens
PFATPMEAEL

SEQ ID NO: 643 - protein, 10 aa, Homo sapiens
PPLPVPGVLL

SEQ ID NO: 644 - protein, 10 aa, Homo sapiens
RGPESRLLEF

SEQ ID NO: 645 - protein, 10 aa, Homo sapiens
FYLAMPFATP

SEQ ID NO: 646 - protein, 10 aa, Homo sapiens
APPLPVPGVL

SEQ ID NO: 647 - protein, 9 aa, Homo sapiens
EFTVSGNIL

SEQ ID NO: 648 - protein, 9 aa, Homo sapiens
PPLPVPGVL

SEQ ID NO: 649 - protein, 9 aa, Homo sapiens
FYLAMPFAT

SEQ ID NO: 650 - protein, 9 aa, Homo sapiens
PLPVPGVLL

SEQ ID NO: 651 - protein, 9 aa, Homo sapiens
SCLQQLSLL

SEQ ID NO: 652 - protein, 10 aa, Homo sapiens
PVPGVLLKEF

SEQ ID NO: 653 - protein, 10 aa, Homo sapiens
FTVSGNILTI

SEQ ID NO: 654 - protein, 10 aa, Homo sapiens
LSISSCLQQL

SEQ ID NO: 655 - protein, 10 aa, Homo sapiens
WITQCFLPVF

SEQ ID NO: 656 - protein, 9 aa, Homo sapiens
EFTVSGNIL

SEQ ID NO: 657 - protein, 9 aa, Homo sapiens
ITQCFLPVF

SEQ ID NO: 658 - protein, 9 aa, Homo sapiens
ESRLLEFYL

SEQ ID NO: 659 - protein, 9 aa, Homo sapiens
EAELARRSL

SEQ ID NO: 660 - protein, 9 aa, Homo sapiens
SISSCLQQL

SEQ ID NO: 661 - protein, 9 aa, Homo sapiens
TVSGNILTI

SEQ ID NO: 662 - protein, 11 aa, Homo sapiens
ATPMEAELARR

SEQ ID NO: 663 - protein, 11 aa, Homo sapiens
FTVSGNILTIR

SEQ ID NO: 664 - protein, 11 aa, Homo sapiens
EAGATGGRGPR

SEQ ID NO: 665 - protein, 11 aa, Homo sapiens
LTIRLTAADHR

SEQ ID NO: 666 - protein, 11 aa, Homo sapiens
RASGPGGGAPR

SEQ ID NO: 667 - protein, 10 aa, Homo sapiens
TVSGNILTIR

SEQ ID NO: 668 - protein, 10 aa, Homo sapiens
ASGPGGGAPR

SEQ ID NO: 669 - protein, 10 aa, Homo sapiens
ATPMEAELAR

SEQ ID NO: 670 - protein, 9 aa, Homo sapiens
VSGNILTIR

SEQ ID NO: 671 - protein, 9 aa, Homo sapiens
PMEAELARR

SEQ ID NO: 672 - protein, 10 aa, Homo sapiens
APRGPHGGAA

SEQ ID NO: 673 - protein, 10 aa, Homo sapiens
APPLPVPGVL

SEQ ID NO: 674 - protein, 10 aa, Homo sapiens
PPLPVPGVLL

SEQ ID NO: 675 - protein, 10 aa, Homo sapiens
GPHGGAASGL

SEQ ID NO: 676 - protein, 10 aa, Homo sapiens
GPRGAGAARA

SEQ ID NO: 677 - protein, 9 aa, Homo sapiens
APRGPHGGA

SEQ ID NO: 678 - protein, 9 aa, Homo sapiens
IPDGPGGNA

SEQ ID NO: 679 - protein, 9 aa, Homo sapiens
APPLPVPGV

SEQ ID NO: 680 - protein, 9 aa, Homo sapiens
PPLPVPGVL

SEQ ID NO: 681 - protein, 9 aa, Homo sapiens
GPGGPGIPD

SEQ ID NO: 682 - protein, 9 aa, Homo sapiens
GPESRLLEF

SEQ ID NO: 683 - protein, 9 aa, Homo sapiens
AADHRQLQL

SEQ ID NO: 684 - protein, 9 aa, Homo sapiens
GARGPESRL

SEQ ID NO: 685 - protein, 9 aa, Homo sapiens
ESRLLEFYL

SEQ ID NO: 686 - protein, 9 aa, Homo sapiens
LLKEFTVSG

SEQ ID NO: 687 - protein, 9 aa, Homo sapiens
SLAQDAPPL

SEQ ID NO: 688 - protein, 9 aa, Homo sapiens
PLPVPGVLL

SEQ ID NO: 689 - protein, 8 aa, Homo sapiens
AELARRSL

SEQ ID NO: 690 - protein, 8 aa, Homo sapiens
LLKEFTVS

SEQ ID NO: 691 - protein, 8 aa, Homo sapiens
PLPVPGVL

SEQ ID NO: 692 - protein, 10 aa, Homo sapiens
SLLMWITQCF

SEQ ID NO: 693 - protein, 10 aa, Homo sapiens
PVPGVLLKEF

SEQ ID NO: 694 - protein, 10 aa, Homo sapiens
LLEFYLAMPF

SEQ ID NO: 695 - protein, 10 aa, Homo sapiens
RLLEFYLAMP

SEQ ID NO: 696 - protein, 10 aa, Homo sapiens
VLLKEFTVSG

SEQ ID NO: 697 - protein, 10 aa, Homo sapiens
MQAEGRGTGG

SEQ ID NO: 698 - protein, 10 aa, Homo sapiens
ILTIRLTAAD

SEQ ID NO: 699 - protein, 10 aa, Homo sapiens
RQLQLSISSC

SEQ ID NO: 700 - protein, 9 aa, Homo sapiens
LLMWITQCF

SEQ ID NO: 701 - protein, 9 aa, Homo sapiens
LLKEFTVSG

SEQ ID NO: 702 - protein, 9 aa, Homo sapiens
PESRLLEFY

SEQ ID NO: 703 - protein, 9 aa, Homo sapiens
LEFYLAMPF

SEQ ID NO: 704 - protein, 9 aa, Homo sapiens
MEAELARRS

SEQ ID NO: 705 - protein, 9 aa, Homo sapiens
ESRLLEFYL

SEQ ID NO: 706 - protein, 9 aa, Homo sapiens
VPGVLLKEF

SEQ ID NO: 707 - protein, 9 aa, Homo sapiens
ITQCFLPVF

SEQ ID NO: 708 - protein, 8 aa, Homo sapiens
PESRLLEF

SEQ ID NO: 709 - protein, 8 aa, Homo sapiens
AELARRSL

SEQ ID NO: 710 - protein, 8 aa, Homo sapiens
PGVLLKEF

SEQ ID NO: 711 - protein, 8 aa, Homo sapiens
MEAELARR

SEQ ID NO: 712 - protein, 10 aa, Homo sapiens
SRLLEFYLAM

SEQ ID NO: 713 - protein, 10 aa, Homo sapiens
RGPESRLLEF

SEQ ID NO: 714 - protein, 10 aa, Homo sapiens
RSLAQDAPPL

SEQ ID NO: 715 - protein, 10 aa, Homo sapiens
GPHGGAASGL

SEQ ID NO: 716 - protein, 10 aa, Homo sapiens
RRSLAQDAPP

SEQ ID NO: 717 - protein, 9 aa, Homo sapiens
ARGPESRLL

SEQ ID NO: 718 - protein, 9 aa, Homo sapiens
IRLTAADHR

SEQ ID NO: 719 - protein, 9 aa, Homo sapiens
GARGPESRL

SEQ ID NO: 720 - protein, 9 aa, Homo sapiens
GRGTGGSTG

SEQ ID NO: 721 - protein, 9 aa, Homo sapiens
GATGGRGPR

SEQ ID NO: 722 - protein, 10 aa, Homo sapiens
PPLPVPGVLL

SEQ ID NO: 723 - protein, 10 aa, Homo sapiens
GPESRLLEFY

SEQ ID NO: 724 - protein, 10 aa, Homo sapiens
GPHGGAASGL

SEQ ID NO: 725 - protein, 10 aa, Homo sapiens
APPLPVPGVL

SEQ ID NO: 726 - protein, 10 aa, Homo sapiens
MPFATPMEAE

SEQ ID NO: 727 - protein, 9 aa, Homo sapiens
PPLPVPGVL

SEQ ID NO: 728 - protein, 9 aa, Homo sapiens
GPESRLLEF

SEQ ID NO: 729 - protein, 9 aa, Homo sapiens
VPGVLLKEF

SEQ ID NO: 730 - protein, 9 aa, Homo sapiens
LQLSISSCL

SEQ ID NO: 731 - protein, 9 aa, Homo sapiens
LPVFLAQPP

SEQ ID NO: 732 - protein, 10 aa, Homo sapiens
RSLAQDAPPL

SEQ ID NO: 733 - protein, 10 aa, Homo sapiens
GARGPESRLL

SEQ ID NO: 734 - protein, 10 aa, Homo sapiens
FTVSGNILTI

SEQ ID NO: 735 - protein, 10 aa, Homo sapiens
LSISSCLQQL

SEQ ID NO: 736 - protein, 10 aa, Homo sapiens
SSCLQQLSLL

SEQ ID NO: 737 - protein, 10 aa, Homo sapiens
VSGNILTIRL

SEQ ID NO: 738 - protein, 10 aa, Homo sapiens
ISSCLQQLSL

SEQ ID NO: 739 - protein, 9 aa, Homo sapiens
EAELARRSL

SEQ ID NO: 740 - protein, 9 aa, Homo sapiens
LTAADHRQL

SEQ ID NO: 741 - protein, 9 aa, Homo sapiens
ESRLLEFYL

SEQ ID NO: 742 - protein, 15 aa, Homo sapiens
EFYLAMPFATPMEAE

SEQ ID NO: 743 - protein, 15 aa, Homo sapiens
SRLLEFYLAMPFATP

SEQ ID NO: 744 - protein, 15 aa, Homo sapiens
ATPMEAELARRSLAQ

SEQ ID NO: 745 - protein, 15 aa, Homo sapiens
GPGIPDGPGGNAGGP

SEQ ID NO: 746 - protein, 15 aa, Homo sapiens
LEFYLAMPFATPMEA

SEQ ID NO: 747 - protein, 15 aa, Homo sapiens
MPFATPMEAELARRS

SEQ ID NO: 748 - protein, 15 aa, Homo sapiens
LLMWITQCFLPVFLA

SEQ ID NO: 749 - protein, 15 aa, Homo sapiens
TQCFLPVFLAQPPSG

SEQ ID NO: 750 - protein, 15 aa, Homo sapiens
QCFLPVFLAQPPSGQ

SEQ ID NO: 751 - protein, 15 aa, Homo sapiens
YLAMPFATPMEAELA

SEQ ID NO: 752 - protein, 15 aa, Homo sapiens
LSLLMWITQCFLPVF

SEQ ID NO: 753 - protein, 15 aa, Homo sapiens
AMPFATPMEAELARR

SEQ ID NO: 754 - protein, 15 aa, Homo sapiens
QLSLLMWITQCFLPV

SEQ ID NO: 755 - protein, 15 aa, Homo sapiens
RRSLAQDAPPLPVPG

SEQ ID NO: 756 - protein, 15 aa, Homo sapiens
QLSISSCLQQLSLLM

SEQ ID NO: 757 - protein, 15 aa, Homo sapiens
SRLLEFYLAMPFATP

SEQ ID NO: 758 - protein, 15 aa, Homo sapiens
PLPVPGVLLKEFTVS

SEQ ID NO: 759 - protein, 15 aa, Homo sapiens
TIRLTAADHRQLQLS

SEQ ID NO: 760 - protein, 15 aa, Homo sapiens
HRQLQLSISSCLQQL

SEQ ID NO: 761 - protein, 15 aa, Homo sapiens
LMWITQCFLPVFLAQ

SEQ ID NO: 762 - protein, 15 aa, Homo sapiens
TIRLTAADHRQLQLS

SEQ ID NO: 763 - protein, 15 aa, Homo sapiens
LSLLMWITQCFLPVF

SEQ ID NO: 764 - protein, 15 aa, Homo sapiens
LLEFYLAMPFATPME

SEQ ID NO: 765 - protein, 15 aa, Homo sapiens
LKEFTVSGNILTIRL

SEQ ID NO: 766 - protein, 15 aa, Homo sapiens
ASGLNGCCRCGARGP

SEQ ID NO: 767 - protein, 15 aa, Homo sapiens
YLAMPFATPMEAELA

SEQ ID NO: 768 - protein, 15 aa, Homo sapiens
ATPMEAELARRSLAQ

SEQ ID NO: 769 - protein, 15 aa, Homo sapiens
PGVLLKEFTVSGNIL

SEQ ID NO: 770 - protein, 15 aa, Homo sapiens
GVLLKEFTVSGNILT

SEQ ID NO: 771 - protein, 15 aa, Homo sapiens
SGNILTIRLTAADHR

SEQ ID NO: 772 - protein, 15 aa, Homo sapiens
HRQLQLSISSCLQQL

SEQ ID NO: 773 - protein, 15 aa, Homo sapiens
AMPFATPMEAELARR

SEQ ID NO: 774 - protein, 15 aa, Homo sapiens
VLLKEFTVSGNILTI

SEQ ID NO: 775 - protein, 15 aa, Homo sapiens
LKEFTVSGNILTIRL

SEQ ID NO: 776 - protein, 15 aa, Homo sapiens
FTVSGNILTIRLTAA

SEQ ID NO: 777 - protein, 15 aa, Homo sapiens
TIRLTAADHRQLQLS

SEQ ID NO: 778 - protein, 15 aa, Homo sapiens
QLSLLMWITQCFLPV

SEQ ID NO: 779 - protein, 15 aa, Homo sapiens
LSLLMWITQCFLPVF

SEQ ID NO: 780 - protein, 15 aa, Homo sapiens
YLAMPFATPMEAELA

SEQ ID NO: 781 - protein, 15 aa, Homo sapiens
SGNILTIRLTAADHR

SEQ ID NO: 782 - protein, 15 aa, Homo sapiens
LEFYLAMPFATPMEA

SEQ ID NO: 783 - protein, 15 aa, Homo sapiens
TQCFLPVFLAQPPSG

SEQ ID NO: 784 - protein, 15 aa, Homo sapiens
ASGLNGCCRCGARGP

SEQ ID NO: 785 - protein, 15 aa, Homo sapiens
SGNILTIRLTAADHR

SEQ ID NO: 786 - protein, 15 aa, Homo sapiens
TIRLTAADHRQLQLS

SEQ ID NO: 787 - protein, 15 aa, Homo sapiens
MPFATPMEAELARRS

SEQ ID NO: 788 - protein, 15 aa, Homo sapiens
ATPMEAELARRSLAQ

SEQ ID NO: 789 - protein, 15 aa, Homo sapiens
TPMEAELARRSLAQD

SEQ ID NO: 790 - protein, 15 aa, Homo sapiens
PMEAELARRSLAQDA

SEQ ID NO: 791 - protein, 15 aa, Homo sapiens
LPVPGVLLKEFTVSG

SEQ ID NO: 792 - protein, 15 aa, Homo sapiens
SRLLEFYLAMPFATP

SEQ ID NO: 793 - protein, 15 aa, Homo sapiens
QCFLPVFLAQPPSGQ

SEQ ID NO: 794 - protein, 15 aa, Homo sapiens
ESRLLEFYLAMPFAT

SEQ ID NO: 795 - protein, 15 aa, Homo sapiens
YLAMPFATPMEAELA

SEQ ID NO: 796 - protein, 15 aa, Homo sapiens
PGVLLKEFTVSGNIL

SEQ ID NO: 797 - protein, 15 aa, Homo sapiens
GVLLKEFTVSGNILT

SEQ ID NO: 798 - protein, 15 aa, Homo sapiens
QLSLLMWITQCFLPV

SEQ ID NO: 799 - protein, 15 aa, Homo sapiens
MWITQCFLPVFLAQP

SEQ ID NO: 800 - protein, 15 aa, Homo sapiens
LLEFYLAMPFATPME

SEQ ID NO: 801 - protein, 15 aa, Homo sapiens
LKEFTVSGNILTIRL

SEQ ID NO: 802 - protein, 264 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVILLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 803 - protein, 11 aa, Homo sapiens
LMEVDPIGHLY

SEQ ID NO: 804 - protein, 11 aa, Homo sapiens
AELVHFLLLKY

SEQ ID NO: 805 - protein, 11 aa, Homo sapiens
QHFVQENYLEY

SEQ ID NO: 806 - protein, 10 aa, Homo sapiens
ASSLPTTMNY

SEQ ID NO: 807 - protein, 10 aa, Homo sapiens
ELVHFLLLKY

SEQ ID NO: 808 - protein, 10 aa, Homo sapiens
LTQHFVQENY

SEQ ID NO: 809 - protein, 9 aa, Homo sapiens
EVDPIGHLY

SEQ ID NO: 810 - protein, 9 aa, Homo sapiens
SSLPTTMNY

SEQ ID NO: 811 - protein, 9 aa, Homo sapiens
LVHFLLLKY

SEQ ID NO: 812 - protein, 9 aa, Homo sapiens
GSVVGNWQY

SEQ ID NO: 813 - protein, 10 aa, Homo sapiens
TLVEVTLGEV

SEQ ID NO: 814 - protein, 10 aa, Homo sapiens
ALVETSYVKV

SEQ ID NO: 815 - protein, 10 aa, Homo sapiens
GLLIIVLAII

SEQ ID NO: 816 - protein, 10 aa, Homo sapiens
AALSRKVAEL

SEQ ID NO: 817 - protein, 10 aa, Homo sapiens
LVFGIELMEV

SEQ ID NO: 818 - protein, 9 aa, Homo sapiens
ALSRKVAEL

SEQ ID NO: 819 - protein, 9 aa, Homo sapiens
LLIIVLAII

SEQ ID NO: 820 - protein, 9 aa, Homo sapiens
GLLIIVLAI

SEQ ID NO: 821 - protein, 9 aa, Homo sapiens
FLWGPRALV

SEQ ID NO: 822 - protein, 9 aa, Homo sapiens
KIWEELSVL

SEQ ID NO: 823 - protein, 10 aa, Homo sapiens
KYRAREPVTK

SEQ ID NO: 824 - protein, 10 aa, Homo sapiens
YVKVLHHMVK

SEQ ID NO: 825 - protein, 10 aa, Homo sapiens
QVPGSDPACY

SEQ ID NO: 826 - protein, 10 aa, Homo sapiens
LLGDNQIMPK

SEQ ID NO: 827 - protein, 10 aa, Homo sapiens
KLLTQHFVQE

SEQ ID NO: 828 - protein, 10 aa, Homo sapiens
FLWGPRALVE

SEQ ID NO: 829 - protein, 9 aa, Homo sapiens
ALVETSYVK

SEQ ID NO: 830 - protein, 9 aa, Homo sapiens
ALGLVGAQA

SEQ ID NO: 831 - protein, 9 aa, Homo sapiens
ELVHFLLLK

SEQ ID NO: 832 - protein, 9 aa, Homo sapiens
YRAREPVTK

SEQ ID NO: 833 - protein, 10 aa, Homo sapiens
ESEFQAALSR

SEQ ID NO: 834 - protein, 10 aa, Homo sapiens
YVKVLHHMVK

SEQ ID NO: 835 - protein, 10 aa, Homo sapiens
AELVHFLLLK

SEQ ID NO: 836 - protein, 10 aa, Homo sapiens
LIIVLAIIAR

SEQ ID NO: 837 - protein, 10 aa, Homo sapiens
ASSSSTLVEV

SEQ ID NO: 838 - protein, 10 aa, Homo sapiens
STLVEVTLGE

SEQ ID NO: 839 - protein, 9 aa, Homo sapiens
ELVHFLLLK

SEQ ID NO: 840 - protein, 9 aa, Homo sapiens
SVLEVFEGR

SEQ ID NO: 841 - protein, 9 aa, Homo sapiens
DSILGDPKK

SEQ ID NO: 842 - protein, 9 aa, Homo sapiens
ALVETSYVK

SEQ ID NO: 843 - protein, 10 aa, Homo sapiens
SYPPLHEWVL

SEQ ID NO: 844 - protein, 10 aa, Homo sapiens
LYIFATCLGL

SEQ ID NO: 845 - protein, 10 aa, Homo sapiens
VFEGREDSIL

SEQ ID NO: 846 - protein, 10 aa, Homo sapiens
KVAELVHFLL

SEQ ID NO: 847 - protein, 9 aa, Homo sapiens
TFPDLESEF

SEQ ID NO: 848 - protein, 9 aa, Homo sapiens
VFEGREDSI

SEQ ID NO: 849 - protein, 9 aa, Homo sapiens
EFLWGPRAL

SEQ ID NO: 850 - protein, 9 aa, Homo sapiens
VAELVHFLL

SEQ ID NO: 851 - protein, 9 aa, Homo sapiens
IFSKASSSL

SEQ ID NO: 852 - protein, 9 aa, Homo sapiens
AELVHFLLL

SEQ ID NO: 853 - protein, 10 aa, Homo sapiens
ELVHFLLLKY

SEQ ID NO: 854 - protein, 10 aa, Homo sapiens
EKIWEELSVL

SEQ ID NO: 855 - protein, 10 aa, Homo sapiens
EVFEGREDSI

SEQ ID NO: 856 - protein, 10 aa, Homo sapiens
EVTLGEVPAA

SEQ ID NO: 857 - protein, 9 aa, Homo sapiens
EVDPIGHLY

SEQ ID NO: 858 - protein, 9 aa, Homo sapiens
LVHFLLLKY

SEQ ID NO: 859 - protein, 9 aa, Homo sapiens
EVFEGREDS

SEQ ID NO: 860 - protein, 9 aa, Homo sapiens
KVAELVHFL

SEQ ID NO: 861 - protein, 9 aa, Homo sapiens
EPVTKAEML

SEQ ID NO: 862 - protein, 9 aa, Homo sapiens
SVVGNWQYF

SEQ ID NO: 863 - protein, 11 aa, Homo sapiens
LLIIVLAIIAR

SEQ ID NO: 864 - protein, 11 aa, Homo sapiens
ELVHFLLLKYR

SEQ ID NO: 865 - protein, 11 aa, Homo sapiens
ELSVLEVFEGR

SEQ ID NO: 866 - protein, 10 aa, Homo sapiens
LIIVLAIIAR

SEQ ID NO: 867 - protein, 10 aa, Homo sapiens
ESEFQAALSR

SEQ ID NO: 868 - protein, 9 aa, Homo sapiens
IIVLAIIAR

SEQ ID NO: 869 - protein, 9 aa, Homo sapiens
ELVHFLLLK

SEQ ID NO: 870 - protein, 9 aa, Homo sapiens
IVLAIIARE

SEQ ID NO: 871 - protein, 9 aa, Homo sapiens
SVLEVFEGR

SEQ ID NO: 872 - protein, 9 aa, Homo sapiens
DSILGDPKK

SEQ ID NO: 873 - protein, 10 aa, Homo sapiens
APEEKIWEEL

SEQ ID NO: 874 - protein, 10 aa, Homo sapiens
SPQGASSLPT

SEQ ID NO: 875 - protein, 10 aa, Homo sapiens
APATEEQEAA

SEQ ID NO: 876 - protein, 10 aa, Homo sapiens
DPIGHLYIFA

SEQ ID NO: 877 - protein, 9 aa, Homo sapiens
GPHISYPPL

SEQ ID NO: 878 - protein, 9 aa, Homo sapiens
LPTTMNYPL

SEQ ID NO: 879 - protein, 9 aa, Homo sapiens
EPVTKAEML

SEQ ID NO: 880 - protein, 9 aa, Homo sapiens
YPPLHEWVL

SEQ ID NO: 881 - protein, 9 aa, Homo sapiens
APATEEQEA

SEQ ID NO: 882 - protein, 9 aa, Homo sapiens
MPKAGLLII

SEQ ID NO: 883 - protein, 9 aa, Homo sapiens
ALSRKVAEL

SEQ ID NO: 884 - protein, 9 aa, Homo sapiens
EPVTKAEML

SEQ ID NO: 885 - protein, 9 aa, Homo sapiens
GLEARGEAL

SEQ ID NO: 886 - protein, 9 aa, Homo sapiens
LLKYRAREP

SEQ ID NO: 887 - protein, 9 aa, Homo sapiens
QIMPKAGLL

SEQ ID NO: 888 - protein, 9 aa, Homo sapiens
EARGEALGL

SEQ ID NO: 889 - protein, 9 aa, Homo sapiens
MPKAGLLII

SEQ ID NO: 890 - protein, 8 aa, Homo sapiens
LLKYRARE

SEQ ID NO: 891 - protein, 8 aa, Homo sapiens
QIMPKAGL

SEQ ID NO: 892 - protein, 8 aa, Homo sapiens
EEKIWEEL

SEQ ID NO: 893 - protein, 10 aa, Homo sapiens
NQEEEGPSTF

SEQ ID NO: 894 - protein, 10 aa, Homo sapiens
ELVHFLLLKY

SEQ ID NO: 895 - protein, 10 aa, Homo sapiens
QVPGSDPACY

SEQ ID NO: 896 - protein, 10 aa, Homo sapiens
SVVGNWQYFF

SEQ ID NO: 897 - protein, 9 aa, Homo sapiens
TQHFVQENY

SEQ ID NO: 898 - protein, 9 aa, Homo sapiens
LVHFLLLKY

SEQ ID NO: 899 - protein, 9 aa, Homo sapiens
FVQENYLEY

SEQ ID NO: 900 - protein, 9 aa, Homo sapiens
WQYFFPVIF

SEQ ID NO: 901 - protein, 9 aa, Homo sapiens
EVDPIGHLY

SEQ ID NO: 902 - protein, 9 aa, Homo sapiens
VVGNWQYFF

SEQ ID NO: 903 - protein, 9 aa, Homo sapiens
EELSVLEVF

SEQ ID NO: 904 - protein, 9 aa, Homo sapiens
QEEEGPSTF

SEQ ID NO: 905 - protein, 9 aa, Homo sapiens
LESEFQAAL

SEQ ID NO: 906 - protein, 9 aa, Homo sapiens
PEEKIWEEL

SEQ ID NO: 907 - protein, 9 aa, Homo sapiens
AELVHFLLL

SEQ ID NO: 908 - protein, 9 aa, Homo sapiens
VETSYVKVL

SEQ ID NO: 909 - protein, 8 aa, Homo sapiens
EEEGPSTF

SEQ ID NO: 910 - protein, 8 aa, Homo sapiens
EEKIWEEL

SEQ ID NO: 911 - protein, 8 aa, Homo sapiens
AELVHFLL

SEQ ID NO: 912 - protein, 8 aa, Homo sapiens
LEARGEAL

SEQ ID NO: 913 - protein, 10 aa, Homo sapiens
AREPVTKAEM

SEQ ID NO: 914 - protein, 10 aa, Homo sapiens
SRKVAELVHF

SEQ ID NO: 915 - protein, 10 aa, Homo sapiens
SEFQAALSRK

SEQ ID NO: 916 - protein, 10 aa, Homo sapiens
RALVETSYVK

SEQ ID NO: 917 - protein, 9 aa, Homo sapiens
YRAREPVTK

SEQ ID NO: 918 - protein, 9 aa, Homo sapiens
PRALVETSY

SEQ ID NO: 919 - protein, 9 aa, Homo sapiens
SRKVAELVH

SEQ ID NO: 920 - protein, 9 aa, Homo sapiens
YFFPVIFSK

SEQ ID NO: 921 - protein, 9 aa, Homo sapiens
KAGLLIIVL

SEQ ID NO: 922 - protein, 9 aa, Homo sapiens
DSILGDPKK

SEQ ID NO: 923 - protein, 10 aa, Homo sapiens
APEEKIWEEL

SEQ ID NO: 924 - protein, 10 aa, Homo sapiens
GPRALVETSY

SEQ ID NO: 925 - protein, 10 aa, Homo sapiens
DPKKLLTQHF

SEQ ID NO: 926 - protein, 9 aa, Homo sapiens
EPVTKAEML

SEQ ID NO: 927 - protein, 9 aa, Homo sapiens
LPTTMNYPL

SEQ ID NO: 928 - protein, 9 aa, Homo sapiens
VPGSDPACY

SEQ ID NO: 929 - protein, 9 aa, Homo sapiens
YPPLHEWVL

SEQ ID NO: 930 - protein, 9 aa, Homo sapiens
GPHISYPPL

SEQ ID NO: 931 - protein, 9 aa, Homo sapiens
DPIGHLYIF

SEQ ID NO: 932 - protein, 9 aa, Homo sapiens
MPKAGLLII

SEQ ID NO: 933 - protein, 10 aa, Homo sapiens
KVAELVHFLL

SEQ ID NO: 934 - protein, 10 aa, Homo sapiens
KASSSLQLVF

SEQ ID NO: 935 - protein, 10 aa, Homo sapiens
SSSTLVEVTL

SEQ ID NO: 936 - protein, 10 aa, Homo sapiens
FSKASSSLQL

SEQ ID NO: 937 - protein, 9 aa, Homo sapiens
KAGLLIIVL

SEQ ID NO: 938 - protein, 9 aa, Homo sapiens
KVAELVHFL

SEQ ID NO: 939 - protein, 9 aa, Homo sapiens
SSTLVEVTL

SEQ ID NO: 940 - protein, 9 aa, Homo sapiens
SSLQLVFGI

SEQ ID NO: 941 - protein, 9 aa, Homo sapiens
KVLHHMVKI

SEQ ID NO: 942 - protein, 9 aa, Homo sapiens
SSLPTTMNY

SEQ ID NO: 943 - protein, 15 aa, Homo sapiens
PACYEFLWGPRALVE

SEQ ID NO: 944 - protein, 15 aa, Homo sapiens
YLEYRQVPGSDPACY

SEQ ID NO: 945 - protein, 15 aa, Homo sapiens
AGLLIIVLAIIAREG

SEQ ID NO: 946 - protein, 15 aa, Homo sapiens
GEALGLVGAQAPATE

SEQ ID NO: 947 - protein, 15 aa, Homo sapiens
QYFFPVIFSKASSSL

SEQ ID NO: 948 - protein, 15 aa, Homo sapiens
SSSLQLVFGIELMEV

SEQ ID NO: 949 - protein, 15 aa, Homo sapiens
EVTLGEVPAAESPDP

SEQ ID NO: 950 - protein, 15 aa, Homo sapiens
HHMVKISGGPHISYP

SEQ ID NO: 951 - protein, 15 aa, Homo sapiens
HFLLLKYRAREPVTK

SEQ ID NO: 952 - protein, 15 aa, Homo sapiens
ETSYVKVLHHMVKIS

SEQ ID NO: 953 - protein, 15 aa, Homo sapiens
EDSILGDPKKLLTQH

SEQ ID NO: 954 - protein, 15 aa, Homo sapiens
IELMEVDPIGHLYIF

SEQ ID NO: 955 - protein, 15 aa, Homo sapiens
YDGLLGDNQIMPKAG

SEQ ID NO: 956 - protein, 15 aa, Homo sapiens
FPDLESEFQAALSRK

SEQ ID NO: 957 - protein, 15 aa, Homo sapiens
GPSTFPDLESEFQAA

SEQ ID NO: 958 - protein, 15 aa, Homo sapiens
LGSVVGNWQYFFPVI

SEQ ID NO: 959 - protein, 15 aa, Homo sapiens
ASSLPTTMNYPLWSQ

SEQ ID NO: 960 - protein, 15 aa, Homo sapiens
VAELVHFLLLKYRAR

SEQ ID NO: 961 - protein, 15 aa, Homo sapiens
CLGLSYDGLLGDNQI

SEQ ID NO: 962 - protein, 15 aa, Homo sapiens
SRKVAELVHFLLLKY

SEQ ID NO: 963 - protein, 15 aa, Homo sapiens
PSTFPDLESEFQAAL

SEQ ID NO: 964 - protein, 15 aa, Homo sapiens
ESEFQAALSRKVAEL

SEQ ID NO: 965 - protein, 15 aa, Homo sapiens
QYFFPVIFSKASSSL

SEQ ID NO: 966 - protein, 15 aa, Homo sapiens
PVIFSKASSSLQLVF

SEQ ID NO: 967 - protein, 15 aa, Homo sapiens
ETSYVKVLHHMVKIS

SEQ ID NO: 968 - protein, 15 aa, Homo sapiens
FPDLESEFQAALSRK

SEQ ID NO: 969 - protein, 15 aa, Homo sapiens
SRKVAELVHFLLLKY

SEQ ID NO: 970 - protein, 15 aa, Homo sapiens
LMEVDPIGHLYIFAT

SEQ ID NO: 971 - protein, 15 aa, Homo sapiens
TSYVKVLHHMVKISG

SEQ ID NO: 972 - protein, 15 aa, Homo sapiens
WQYFFPVIFSKASSS

SEQ ID NO: 973 - protein, 15 aa, Homo sapiens
ESEFQAALSRKVAEL

SEQ ID NO: 974 - protein, 15 aa, Homo sapiens
ASSLPTTMNYPLWSQ

SEQ ID NO: 975 - protein, 15 aa, Homo sapiens
ATCLGLSYDGLLGDN

SEQ ID NO: 976 - protein, 15 aa, Homo sapiens
QYFFPVIFSKASSSL

SEQ ID NO: 977 - protein, 15 aa, Homo sapiens
FPVIFSKASSSLQLV

SEQ ID NO: 978 - protein, 15 aa, Homo sapiens
PVIFSKASSSLQLVF

SEQ ID NO: 979 - protein, 15 aa, Homo sapiens
GHLYIFATCLGLSYD

SEQ ID NO: 980 - protein, 15 aa, Homo sapiens
LEVFEGREDSILGDP

SEQ ID NO: 981 - protein, 15 aa, Homo sapiens
PRALVETSYVKVLHH

SEQ ID NO: 982 - protein, 15 aa, Homo sapiens
HISYPPLHEWVLREG

SEQ ID NO: 983 - protein, 15 aa, Homo sapiens
VKVLHHMVKISGGPH

SEQ ID NO: 984 - protein, 15 aa, Homo sapiens
WQYFFPVIFSKASSS

SEQ ID NO: 985 - protein, 15 aa, Homo sapiens
PACYEFLWGPRALVE

SEQ ID NO: 986 - protein, 15 aa, Homo sapiens
ETSYVKVLHHMVKIS

SEQ ID NO: 987 - protein, 15 aa, Homo sapiens
SRKVAELVHFLLLKY

SEQ ID NO: 988 - protein, 15 aa, Homo sapiens
ELVHFLLLKYRAREP

SEQ ID NO: 989 - protein, 15 aa, Homo sapiens
QYFFPVIFSKASSSL

SEQ ID NO: 990 - protein, 15 aa, Homo sapiens
YLEYRQVPGSDPACY

SEQ ID NO: 991 - protein, 15 aa, Homo sapiens
TSYVKVLHHMVKISG

SEQ ID NO: 992 - protein, 15 aa, Homo sapiens
SEFQAALSRKVAELV

SEQ ID NO: 993 - protein, 15 aa, Homo sapiens
GSVVGNWQYFFPVIF

SEQ ID NO: 994 - protein, 15 aa, Homo sapiens
HFLLLKYRAREPVTK

SEQ ID NO: 995 - protein, 15 aa, Homo sapiens
IGHLYIFATCLGLSY

SEQ ID NO: 996 - protein, 15 aa, Homo sapiens
VAELVHFLLLKYRAR

SEQ ID NO: 997 - protein, 15 aa, Homo sapiens
SSSLQLVFGIELMEV

SEQ ID NO: 998 - protein, 15 aa, Homo sapiens
GIELMEVDPIGHLYI

SEQ ID NO: 999 - protein, 15 aa, Homo sapiens
TCLGLSYDGLLGDNQ

SEQ ID NO: 1000 - protein, 15 aa, Homo sapiens
DNQIMPKAGLLIIVL

SEQ ID NO: 1001 - protein, 15 aa, Homo sapiens
AGLLIIVLAIIAREG

SEQ ID NO: 1002 - protein, 15 aa, Homo sapiens
LSVLEVFEGREDSIL

SEQ ID NO: 1003 - protein, 317 aa, Homo sapiens
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 1004 - protein, 11 aa, Homo sapiens
YTLVTCLGLSY

SEQ ID NO: 1005 - protein, 11 aa, Homo sapiens
VKEVDPASNTY

SEQ ID NO: 1006 - protein, 11 aa, Homo sapiens
IWEELGVMGVY

SEQ ID NO: 1007 - protein, 11 aa, Homo sapiens
QDWVQENYLEY

SEQ ID NO: 1008 - protein, 10 aa, Homo sapiens
VYDGREHTVY

SEQ ID NO: 1009 - protein, 10 aa, Homo sapiens
WEELGVMGVY

SEQ ID NO: 1010 - protein, 10 aa, Homo sapiens
LTQDWVQENY

SEQ ID NO: 1011 - protein, 9 aa, Homo sapiens
EVDPASNTY

SEQ ID NO: 1012 - protein, 9 aa, Homo sapiens
TQDWVQENY

SEQ ID NO: 1013 - protein, 9 aa, Homo sapiens
MLERVIKNY

SEQ ID NO: 1014 - protein, 10 aa, Homo sapiens
ALAETSYVKV

SEQ ID NO: 1015 - protein, 10 aa, Homo sapiens
GLLIIVLGTI

SEQ ID NO: 1016 - protein, 10 aa, Homo sapiens
MIFGIDVKEV

SEQ ID NO: 1017 - protein, 10 aa, Homo sapiens
PLVPGTLEEV

SEQ ID NO: 1018 - protein, 10 aa, Homo sapiens
VIFGKASESL

SEQ ID NO: 1019 - protein, 9 aa, Homo sapiens
ALSNKVDEL

SEQ ID NO: 1020 - protein, 9 aa, Homo sapiens
LLIIVLGTI

SEQ ID NO: 1021 - protein, 9 aa, Homo sapiens
ALLEEEEGV

SEQ ID NO: 1022 - protein, 9 aa, Homo sapiens
KVLEHVVRV

SEQ ID NO: 1023 - protein, 9 aa, Homo sapiens
QIFPKTGLL

SEQ ID NO: 1024 - protein, 10 aa, Homo sapiens
SLFREALSNK

SEQ ID NO: 1025 - protein, 10 aa, Homo sapiens
KYRAKELVTK

SEQ ID NO: 1026 - protein, 10 aa, Homo sapiens
RVRIAYPSLR

SEQ ID NO: 1027 - protein, 10 aa, Homo sapiens
TLVTCLGLSY

SEQ ID NO: 1028 - protein, 10 aa, Homo sapiens
QVPGSNPARY

SEQ ID NO: 1029 - protein, 10 aa, Homo sapiens
HVVRVNARVR

SEQ ID NO: 1030 - protein, 9 aa, Homo sapiens
ALAETSYVK

SEQ ID NO: 1031 - protein, 9 aa, Homo sapiens
FLLRKYRAK

SEQ ID NO: 1032 - protein, 9 aa, Homo sapiens
ALGLVGAQA

SEQ ID NO: 1033 - protein, 9 aa, Homo sapiens
ELAHFLLRK

SEQ ID NO: 1034 - protein, 10 aa, Homo sapiens
TSPDAESLFR

SEQ ID NO: 1035 - protein, 10 aa, Homo sapiens
YVKVLEHVVR

SEQ ID NO: 1036 - protein, 10 aa, Homo sapiens
SSEQKSQHCK

SEQ ID NO: 1037 - protein, 10 aa, Homo sapiens
LVTKAEMLER

SEQ ID NO: 1038 - protein, 10 aa, Homo sapiens
RVRIAYPSLR

SEQ ID NO: 1039 - protein, 9 aa, Homo sapiens
VTKAEMLER

SEQ ID NO: 1040 - protein, 9 aa, Homo sapiens
ELAHFLLRK

SEQ ID NO: 1041 - protein, 9 aa, Homo sapiens
GVMGVYDGR

SEQ ID NO: 1042 - protein, 9 aa, Homo sapiens
TTISFTCWR

SEQ ID NO: 1043 - protein, 9 aa, Homo sapiens
ALAETSYVK

SEQ ID NO: 1044 - protein, 10 aa, Homo sapiens
AYPSLREAAL

SEQ ID NO: 1045 - protein, 10 aa, Homo sapiens
TYTLVTCLGL

SEQ ID NO: 1046 - protein, 10 aa, Homo sapiens
NYKRCFPVIF

SEQ ID NO: 1047 - protein, 10 aa, Homo sapiens
IFPKTGLLII

SEQ ID NO: 1048 - protein, 10 aa, Homo sapiens
KVDELAHFLL

SEQ ID NO: 1049 - protein, 9 aa, Homo sapiens
VYGEPRKLL

SEQ ID NO: 1050 - protein, 9 aa, Homo sapiens
NYKRCFPVI

SEQ ID NO: 1051 - protein, 9 aa, Homo sapiens
EFLWGPRAL

SEQ ID NO: 1052 - protein, 9 aa, Homo sapiens
IFGKASESL

SEQ ID NO: 1053 - protein, 10 aa, Homo sapiens
EMLERVIKNY

SEQ ID NO: 1054 - protein, 10 aa, Homo sapiens
EGVEAQEEAL

SEQ ID NO: 1055 - protein, 10 aa, Homo sapiens
ELAHFLLRKY

SEQ ID NO: 1056 - protein, 10 aa, Homo sapiens
EALSNKVDEL

SEQ ID NO: 1057 - protein, 10 aa, Homo sapiens
DWVQENYLEY

SEQ ID NO: 1058 - protein, 10 aa, Homo sapiens
ETSYVKVLEH

SEQ ID NO: 1059 - protein, 9 aa, Homo sapiens
EVDPASNTY

SEQ ID NO: 1060 - protein, 9 aa, Homo sapiens
LVTCLGLSY

SEQ ID NO: 1061 - protein, 9 aa, Homo sapiens
ELVTKAEML

SEQ ID NO: 1062 - protein, 9 aa, Homo sapiens
WVQENYLEY

SEQ ID NO: 1063 - protein, 11 aa, Homo sapiens
ELAHFLLRKYR

SEQ ID NO: 1064 - protein, 11 aa, Homo sapiens
STSPDAESLFR

SEQ ID NO: 1065 - protein, 11 aa, Homo sapiens
ELVTKAEMLER

SEQ ID NO: 1066 - protein, 10 aa, Homo sapiens
YVKVLEHVVR

SEQ ID NO: 1067 - protein, 10 aa, Homo sapiens
PTTISFTCWR

SEQ ID NO: 1068 - protein, 10 aa, Homo sapiens
LVTKAEMLER

SEQ ID NO: 1069 - protein, 9 aa, Homo sapiens
ELAHFLLRK

SEQ ID NO: 1070 - protein, 9 aa, Homo sapiens
TTISFTCWR

SEQ ID NO: 1071 - protein, 9 aa, Homo sapiens
GVMGVYDGR

SEQ ID NO: 1072 - protein, 9 aa, Homo sapiens
QVPGSNPAR

SEQ ID NO: 1073 - protein, 10 aa, Homo sapiens
YPSLREAALL

SEQ ID NO: 1074 - protein, 10 aa, Homo sapiens
SPQGASALPT

SEQ ID NO: 1075 - protein, 10 aa, Homo sapiens
VPGTLEEVPA

SEQ ID NO: 1076 - protein, 10 aa, Homo sapiens
APTTEEQEAA

SEQ ID NO: 1077 - protein, 10 aa, Homo sapiens
DPASNTYTLV

SEQ ID NO: 1078 - protein, 10 aa, Homo sapiens
PPQSPQGASA

SEQ ID NO: 1079 - protein, 9 aa, Homo sapiens
YPSLREAAL

SEQ ID NO: 1080 - protein, 9 aa, Homo sapiens
DPASNTYTL

SEQ ID NO: 1081 - protein, 9 aa, Homo sapiens
APTTEEQEA

SEQ ID NO: 1082 - protein, 9 aa, Homo sapiens
FPKTGLLII

SEQ ID NO: 1083 - protein, 9 aa, Homo sapiens
LRKYRAKEL

SEQ ID NO: 1084 - protein, 9 aa, Homo sapiens
ALSNKVDEL

SEQ ID NO: 1085 - protein, 9 aa, Homo sapiens
ELVTKAEML

SEQ ID NO: 1086 - protein, 9 aa, Homo sapiens
YPSLREAAL

SEQ ID NO: 1087 - protein, 9 aa, Homo sapiens
QIFPKTGLL

SEQ ID NO: 1088 - protein, 9 aa, Homo sapiens
VIKNYKRCF

SEQ ID NO: 1089 - protein, 8 aa, Homo sapiens
SLREAALL

SEQ ID NO: 1090 - protein, 8 aa, Homo sapiens
SLKMIFGI

SEQ ID NO: 1091 - protein, 8 aa, Homo sapiens
QIFPKTGL

SEQ ID NO: 1092 - protein, 8 aa, Homo sapiens
FPKTGLLI

SEQ ID NO: 1093 - protein, 10 aa, Homo sapiens
TLVTCLGLSY

SEQ ID NO: 1094 - protein, 10 aa, Homo sapiens
RVNARVRIAY

SEQ ID NO: 1095 - protein, 10 aa, Homo sapiens
ELAHFLLRKY

SEQ ID NO: 1096 - protein, 10 aa, Homo sapiens
QVPGSNPARY

SEQ ID NO: 1097 - protein, 10 aa, Homo sapiens
RVIKNYKRCF

SEQ ID NO: 1098 - protein, 9 aa, Homo sapiens
MLERVIKNY

SEQ ID NO: 1099 - protein, 9 aa, Homo sapiens
TQDWVQENY

SEQ ID NO: 1100 - protein, 9 aa, Homo sapiens
LVTCLGLSY

SEQ ID NO: 1101 - protein, 9 aa, Homo sapiens
WVQENYLEY

SEQ ID NO: 1102 - protein, 9 aa, Homo sapiens
EVDPASNTY

SEQ ID NO: 1103 - protein, 9 aa, Homo sapiens
AESLFREAL

SEQ ID NO: 1104 - protein, 9 aa, Homo sapiens
SEEEIWEEL

SEQ ID NO: 1105 - protein, 9 aa, Homo sapiens
EELGVMGVY

SEQ ID NO: 1106 - protein, 9 aa, Homo sapiens
AETSYVKVL

SEQ ID NO: 1107 - protein, 8 aa, Homo sapiens
DELAHFLL

SEQ ID NO: 1108 - protein, 8 aa, Homo sapiens
EEEIWEEL

SEQ ID NO: 1109 - protein, 8 aa, Homo sapiens
LERVIKNY

SEQ ID NO: 1110 - protein, 8 aa, Homo sapiens
SESLKMIF

SEQ ID NO: 1111 - protein, 8 aa, Homo sapiens
VEAQEEAL

SEQ ID NO: 1112 - protein, 8 aa, Homo sapiens
DGREHTVY

SEQ ID NO: 1113 - protein, 10 aa, Homo sapiens
KRCFPVIFGK

SEQ ID NO: 1114 - protein, 10 aa, Homo sapiens
ARYEFLWGPR

SEQ ID NO: 1115 - protein, 10 aa, Homo sapiens
ARVRIAYPSL

SEQ ID NO: 1116 - protein, 9 aa, Homo sapiens
YRAKELVTK

SEQ ID NO: 1117 - protein, 9 aa, Homo sapiens
ERVIKNYKR

SEQ ID NO: 1118 - protein, 9 aa, Homo sapiens
VRIAYPSLR

SEQ ID NO: 1119 - protein, 9 aa, Homo sapiens
LRKYRAKEL

SEQ ID NO: 1120 - protein, 9 aa, Homo sapiens
RCFPVIFGK

SEQ ID NO: 1121 - protein, 9 aa, Homo sapiens
PRALAETSY

SEQ ID NO: 1122 - protein, 9 aa, Homo sapiens
KMIFGIDVK

SEQ ID NO: 1123 - protein, 10 aa, Homo sapiens
YPSLREAALL

SEQ ID NO: 1124 - protein, 10 aa, Homo sapiens
GPRALAETSY

SEQ ID NO: 1125 - protein, 9 aa, Homo sapiens
DPASNTYTL

SEQ ID NO: 1126 - protein, 9 aa, Homo sapiens
YPSLREAAL

SEQ ID NO: 1127 - protein, 9 aa, Homo sapiens
VPGSNPARY

SEQ ID NO: 1128 - protein, 9 aa, Homo sapiens
FPKTGLLII

SEQ ID NO: 1129 - protein, 9 aa, Homo sapiens
LPTTISFTC

SEQ ID NO: 1130 - protein, 9 aa, Homo sapiens
KVDELAHFL

SEQ ID NO: 1131 - protein, 9 aa, Homo sapiens
MLERVIKNY

SEQ ID NO: 1132 - protein, 9 aa, Homo sapiens
LGLSYDGLL

SEQ ID NO: 1133 - protein, 10 aa, Homo sapiens
RVIKNYKRCF

SEQ ID NO: 1134 - protein, 10 aa, Homo sapiens
KASESLKMIF

SEQ ID NO: 1135 - protein, 10 aa, Homo sapiens
SSSPLVPGTL

SEQ ID NO: 1136 - protein, 9 aa, Homo sapiens
KAEMLERVI

SEQ ID NO: 1137 - protein, 9 aa, Homo sapiens
KTGLLIIVL

SEQ ID NO: 1138 - protein, 9 aa, Homo sapiens
KVDELAHFL

SEQ ID NO: 1139 - protein, 9 aa, Homo sapiens
KASESLKMI

SEQ ID NO: 1140 - protein, 9 aa, Homo sapiens
PSLREAALL

SEQ ID NO: 1141 - protein, 9 aa, Homo sapiens
SSPLVPGTL

SEQ ID NO: 1142 - protein, 9 aa, Homo sapiens
LAHFLLRKY

SEQ ID NO: 1143 - protein, 15 aa, Homo sapiens
PARYEFLWGPRALAE

SEQ ID NO: 1144 - protein, 15 aa, Homo sapiens
TGLLIIVLGTIAMEG

SEQ ID NO: 1145 - protein, 15 aa, Homo sapiens
YLEYRQVPGSNPARY

SEQ ID NO: 1146 - protein, 15 aa, Homo sapiens
KRCFPVIFGKASESL

SEQ ID NO: 1147 - protein, 15 aa, Homo sapiens
EEALGLVGAQAPTTE

SEQ ID NO: 1148 - protein, 15 aa, Homo sapiens
SESLKMIFGIDVKEV

SEQ ID NO: 1149 - protein, 15 aa, Homo sapiens
GLLIIVLGTIAMEGD

SEQ ID NO: 1150 - protein, 15 aa, Homo sapiens
PGTLEEVPAAESAGP

SEQ ID NO: 1151 - protein, 15 aa, Homo sapiens
HFLLRKYRAKELVTK

SEQ ID NO: 1152 - protein, 15 aa, Homo sapiens
EEIWEELGVMGVYDG

SEQ ID NO: 1153 - protein, 15 aa, Homo sapiens
GPSTSPDAESLFREA

SEQ ID NO: 1154 - protein, 15 aa, Homo sapiens
EHTVYGEPRKLLTQD

SEQ ID NO: 1155 - protein, 15 aa, Homo sapiens
LERVIKNYKRCFPVI

SEQ ID NO: 1156 - protein, 15 aa, Homo sapiens
VVRVNARVRIAYPSL

SEQ ID NO: 1157 - protein, 15 aa, Homo sapiens
KMIFGIDVKEVDPAS

SEQ ID NO: 1158 - protein, 15 aa, Homo sapiens
KAEMLERVIKNYKRC

SEQ ID NO: 1159 - protein, 15 aa, Homo sapiens
CLGLSYDGLLGNNQI

SEQ ID NO: 1160 - protein, 15 aa, Homo sapiens
RKLLTQDWVQENYLE

SEQ ID NO: 1161 - protein, 15 aa, Homo sapiens
ALSNKVDELAHFLLR

SEQ ID NO: 1162 - protein, 15 aa, Homo sapiens
TYTLVTCLGLSYDGL

SEQ ID NO: 1163 - protein, 15 aa, Homo sapiens
ESLFREALSNKVDEL

SEQ ID NO: 1164 - protein, 15 aa, Homo sapiens
SNKVDELAHFLLRKY

SEQ ID NO: 1165 - protein, 15 aa, Homo sapiens
VKEVDPASNTYTLVT

SEQ ID NO: 1166 - protein, 15 aa, Homo sapiens
GLLIIVLGTIAMEGD

SEQ ID NO: 1167 - protein, 15 aa, Homo sapiens
EHTVYGEPRKLLTQD

SEQ ID NO: 1168 - protein, 15 aa, Homo sapiens
VKVLEHVVRVNARVR

SEQ ID NO: 1169 - protein, 15 aa, Homo sapiens
KRCFPVIFGKASESL

SEQ ID NO: 1170 - protein, 15 aa, Homo sapiens
PVIFGKASESLKMIF

SEQ ID NO: 1171 - protein, 15 aa, Homo sapiens
SNTYTLVTCLGLSYD

SEQ ID NO: 1172 - protein, 15 aa, Homo sapiens
GLSYDGLLGNNQIFP

SEQ ID NO: 1173 - protein, 15 aa, Homo sapiens
ESLFREALSNKVDEL

SEQ ID NO: 1174 - protein, 15 aa, Homo sapiens
VTCLGLSYDGLLGNN

SEQ ID NO: 1175 - protein, 15 aa, Homo sapiens
NQIFPKTGLLIIVLG

SEQ ID NO: 1176 - protein, 15 aa, Homo sapiens
ASALPTTISFTCWRQ

SEQ ID NO: 1177 - protein, 15 aa, Homo sapiens
FPVIFGKASESLKMI

SEQ ID NO: 1178 - protein, 15 aa, Homo sapiens
GLLIIVLGTIAMEGD

SEQ ID NO: 1179 - protein, 15 aa, Homo sapiens
PRALAETSYVKVLEH

SEQ ID NO: 1180 - protein, 15 aa, Homo sapiens
ETSYVKVLEHVVRVN

SEQ ID NO: 1181 - protein, 15 aa, Homo sapiens
RIAYPSLREAALLEE

SEQ ID NO: 1182 - protein, 15 aa, Homo sapiens
EQEAAVSSSSPLVPG

SEQ ID NO: 1183 - protein, 15 aa, Homo sapiens
VKVLEHVVRVNARVR

SEQ ID NO: 1184 - protein, 15 aa, Homo sapiens
RYEFLWGPRALAETS

SEQ ID NO: 1185 - protein, 15 aa, Homo sapiens
PARYEFLWGPRALAE

SEQ ID NO: 1186 - protein, 15 aa, Homo sapiens
ELAHFLLRKYRAKEL

SEQ ID NO: 1187 - protein, 15 aa, Homo sapiens
KRCFPVIFGKASESL

SEQ ID NO: 1188 - protein, 15 aa, Homo sapiens
YLEYRQVPGSNPARY

SEQ ID NO: 1189 - protein, 15 aa, Homo sapiens
TSYVKVLEHVVRVNA

SEQ ID NO: 1190 - protein, 15 aa, Homo sapiens
SNKVDELAHFLLRKY

SEQ ID NO: 1191 - protein, 15 aa, Homo sapiens
KMIFGIDVKEVDPAS

SEQ ID NO: 1192 - protein, 15 aa, Homo sapiens
AEMLERVIKNYKRCF

SEQ ID NO: 1193 - protein, 15 aa, Homo sapiens
HFLLRKYRAKELVTK

SEQ ID NO: 1194 - protein, 15 aa, Homo sapiens
LGVMGVYDGREHTVY

SEQ ID NO: 1195 - protein, 15 aa, Homo sapiens
EAAVSSSSPLVPGTL

SEQ ID NO: 1196 - protein, 15 aa, Homo sapiens
ASALPTTISFTCWRQ

SEQ ID NO: 1197 - protein, 15 aa, Homo sapiens
ERVIKNYKRCFPVIF

SEQ ID NO: 1198 - protein, 15 aa, Homo sapiens
SESLKMIFGIDVKEV

SEQ ID NO: 1199 - protein, 15 aa, Homo sapiens
TCLGLSYDGLLGNNQ

SEQ ID NO: 1200 - protein, 15 aa, Homo sapiens
NNQIFPKTGLLIIVL

SEQ ID NO: 1201 - protein, 15 aa, Homo sapiens
GLLIIVLGTIAMEGD

SEQ ID NO: 1202 - protein, 15 aa, Homo sapiens
LIIVLGTIAMEGDSA

SEQ ID NO: 1203 - protein, 188 aa, Homo sapiens
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGPKRGEHAWTHRLRERKQLVIYEEISDPEEDDE

SEQ ID NO: 1204 - protein, 11 aa, Homo sapiens
RLRERKQLVIY

SEQ ID NO: 1205 - protein, 11 aa, Homo sapiens
EKMKASEKIFY

SEQ ID NO: 1206 - protein, 11 aa, Homo sapiens
KIFYVYMKRKY

SEQ ID NO: 1207 - protein, 11 aa, Homo sapiens
IQKAFDDIAKY

SEQ ID NO: 1208 - protein, 11 aa, Homo sapiens
MKASEKIFYVY

SEQ ID NO: 1209 - protein, 10 aa, Homo sapiens
LRERKQLVIY

SEQ ID NO: 1210 - protein, 10 aa, Homo sapiens
IFYVYMKRKY

SEQ ID NO: 1211 - protein, 9 aa, Homo sapiens
ASEKIFYVY

SEQ ID NO: 1212 - protein, 9 aa, Homo sapiens
KAFDDIAKY

SEQ ID NO: 1213 - protein, 9 aa, Homo sapiens
FYVYMKRKY

SEQ ID NO: 1214 - protein, 10 aa, Homo sapiens
RLRERKQLVI

SEQ ID NO: 1215 - protein, 10 aa, Homo sapiens
QMTFGRLQGI

SEQ ID NO: 1216 - protein, 9 aa, Homo sapiens
RLQGISPKI

SEQ ID NO: 1217 - protein, 9 aa, Homo sapiens
KASEKIFYV

SEQ ID NO: 1218 - protein, 9 aa, Homo sapiens
RLRERKQLV

SEQ ID NO: 1219 - protein, 9 aa, Homo sapiens
QIPEKIQKA

SEQ ID NO: 1220 - protein, 9 aa, Homo sapiens
MTFGRLQGI

SEQ ID NO: 1221 - protein, 9 aa, Homo sapiens
TKLGFKATL

SEQ ID NO: 1222 - protein, 9 aa, Homo sapiens
DAFARRPTV

SEQ ID NO: 1223 - protein, 9 aa, Homo sapiens
KIQKAFDDI

SEQ ID NO: 1224 - protein, 10 aa, Homo sapiens
RLRERKQLVI

SEQ ID NO: 1225 - protein, 10 aa, Homo sapiens
KIFYVYMKRK

SEQ ID NO: 1226 - protein, 9 aa, Homo sapiens
KIHERSGPK

SEQ ID NO: 1227 - protein, 9 aa, Homo sapiens
QVERPQMTF

SEQ ID NO: 1228 - protein, 9 aa, Homo sapiens
TLPPFMCNK

SEQ ID NO: 1229 - protein, 9 aa, Homo sapiens
GISPKIMPK

SEQ ID NO: 1230 - protein, 9 aa, Homo sapiens
TVGAQIPEK

SEQ ID NO: 1231 - protein, 9 aa, Homo sapiens
AQIPEKIQK

SEQ ID NO: 1232 - protein, 9 aa, Homo sapiens
KRKYEAMTK

SEQ ID NO: 1233 - protein, 10 aa, Homo sapiens
ARRPTVGAQI

SEQ ID NO: 1234 - protein, 10 aa, Homo sapiens
ATLPPFMCNK

SEQ ID NO: 1235 - protein, 10 aa, Homo sapiens
PTVGAQIPEK

SEQ ID NO: 1236 - protein, 10 aa, Homo sapiens
PTTSEKIHER

SEQ ID NO: 1237 - protein, 10 aa, Homo sapiens
FSKEEWEKMK

SEQ ID NO: 1238 - protein, 9 aa, Homo sapiens
ASGPQNDGK

SEQ ID NO: 1239 - protein, 9 aa, Homo sapiens
TVGAQIPEK

SEQ ID NO: 1240 - protein, 9 aa, Homo sapiens
GISPKIMPK

SEQ ID NO: 1241 - protein, 9 aa, Homo sapiens
TTSEKIHER

SEQ ID NO: 1242 - protein, 9 aa, Homo sapiens
WTHRLRERK

SEQ ID NO: 1243 - protein, 9 aa, Homo sapiens
AQIPEKIQK

SEQ ID NO: 1244 - protein, 10 aa, Homo sapiens
KYEAMTKLGF

SEQ ID NO: 1245 - protein, 10 aa, Homo sapiens
TVGAQIPEKI

SEQ ID NO: 1246 - protein, 10 aa, Homo sapiens
QIPEKIQKAF

SEQ ID NO: 1247 - protein, 10 aa, Homo sapiens
VYMKRKYEAM

SEQ ID NO: 1248 - protein, 10 aa, Homo sapiens
GRLQGISPKI

SEQ ID NO: 1249 - protein, 9 aa, Homo sapiens
AFDDIAKYF

SEQ ID NO: 1250 - protein, 9 aa, Homo sapiens
VGAQIPEKI

SEQ ID NO: 1251 - protein, 9 aa, Homo sapiens
GFKATLPPF

SEQ ID NO: 1252 - protein, 9 aa, Homo sapiens
GKPTTSEKI

SEQ ID NO: 1253 - protein, 9 aa, Homo sapiens
KQLVIYEEI

SEQ ID NO: 1254 - protein, 10 aa, Homo sapiens
EVPEASGPQN

SEQ ID NO: 1255 - protein, 10 aa, Homo sapiens
ERPQMTFGRL

SEQ ID NO: 1256 - protein, 10 aa, Homo sapiens
LVIYEEISDP

SEQ ID NO: 1257 - protein, 10 aa, Homo sapiens
EKMKASEKIF

SEQ ID NO: 1258 - protein, 10 aa, Homo sapiens
MTKLGFKATL

SEQ ID NO: 1259 - protein, 9 aa, Homo sapiens
KAFDDIAKY

SEQ ID NO: 1260 - protein, 9 aa, Homo sapiens
EKIFYVYMK

SEQ ID NO: 1261 - protein, 9 aa, Homo sapiens
QVERPQMTF

SEQ ID NO: 1262 - protein, 9 aa, Homo sapiens
EAMTKLGFK

SEQ ID NO: 1263 - protein, 9 aa, Homo sapiens
MTFGRLQGI

SEQ ID NO: 1264 - protein, 11 aa, Homo sapiens
ATLPPFMCNKR

SEQ ID NO: 1265 - protein, 11 aa, Homo sapiens
KIQKAFDDIAK

SEQ ID NO: 1266 - protein, 11 aa, Homo sapiens
QVERPQMTFGR

SEQ ID NO: 1267 - protein, 10 aa, Homo sapiens
GAQIPEKIQK

SEQ ID NO: 1268 - protein, 10 aa, Homo sapiens
DPNRGNQVER

SEQ ID NO: 1269 - protein, 10 aa, Homo sapiens
EASGPQNDGK

SEQ ID NO: 1270 - protein, 9 aa, Homo sapiens
TTSEKIHER

SEQ ID NO: 1271 - protein, 9 aa, Homo sapiens
EAMTKLGFK

SEQ ID NO: 1272 - protein, 9 aa, Homo sapiens
TVGAQIPEK

SEQ ID NO: 1273 - protein, 9 aa, Homo sapiens
GISPKIMPK

SEQ ID NO: 1274 - protein, 10 aa, Homo sapiens
SPKIMPKKPA

SEQ ID NO: 1275 - protein, 10 aa, Homo sapiens
GPKRGEHAWT

SEQ ID NO: 1276 - protein, 10 aa, Homo sapiens
LPPFMCNKRA

SEQ ID NO: 1277 - protein, 9 aa, Homo sapiens
RPQMTFGRL

SEQ ID NO: 1278 - protein, 9 aa, Homo sapiens
GPQNDGKEL

SEQ ID NO: 1279 - protein, 9 aa, Homo sapiens
IPEKIQKAF

SEQ ID NO: 1280 - protein, 9 aa, Homo sapiens
PPGKPTTSE

SEQ ID NO: 1281 - protein, 9 aa, Homo sapiens
FARRPTVGA

SEQ ID NO: 1282 - protein, 9 aa, Homo sapiens
AEDFQGNDL

SEQ ID NO: 1283 - protein, 9 aa, Homo sapiens
MPKKPAEEG

SEQ ID NO: 1284 - protein, 9 aa, Homo sapiens
WEKMKASEK

SEQ ID NO: 1285 - protein, 9 aa, Homo sapiens
YMKRKYEAM

SEQ ID NO: 1286 - protein, 9 aa, Homo sapiens
SPKIMPKKP

SEQ ID NO: 1287 - protein, 9 aa, Homo sapiens
GPQNDGKEL

SEQ ID NO: 1288 - protein, 8 aa, Homo sapiens
RLRERKQL

SEQ ID NO: 1289 - protein, 8 aa, Homo sapiens
KLGFKATL

SEQ ID NO: 1290 - protein, 8 aa, Homo sapiens
EAMTKLGF

SEQ ID NO: 1291 - protein, 8 aa, Homo sapiens
IQKAFDDI

SEQ ID NO: 1292 - protein, 8 aa, Homo sapiens
GPKRGEHA

SEQ ID NO: 1293 - protein, 9 aa, Homo sapiens
GFKATLPPF

SEQ ID NO: 1294 - protein, 10 aa, Homo sapiens
NQVERPQMTF

SEQ ID NO: 1295 - protein, 10 aa, Homo sapiens
AQIPEKIQKA

SEQ ID NO: 1296 - protein, 10 aa, Homo sapiens
KASEKIFYVY

SEQ ID NO: 1297 - protein, 10 aa, Homo sapiens
RLRERKQLVI

SEQ ID NO: 1298 - protein, 10 aa, Homo sapiens
RLQGISPKIM

SEQ ID NO: 1299 - protein, 10 aa, Homo sapiens
ELCPPGKPTT

SEQ ID NO: 1300 - protein, 9 aa, Homo sapiens
KAFDDIAKY

SEQ ID NO: 1301 - protein, 9 aa, Homo sapiens
AQIPEKIQK

SEQ ID NO: 1302 - protein, 9 aa, Homo sapiens
PQNDGKELC

SEQ ID NO: 1303 - protein, 9 aa, Homo sapiens
RLRERKQLV

SEQ ID NO: 1304 - protein, 9 aa, Homo sapiens
RERKQLVIY

SEQ ID NO: 1305 - protein, 9 aa, Homo sapiens
YEAMTKLGF

SEQ ID NO: 1306 - protein, 9 aa, Homo sapiens
AEDFQGNDL

SEQ ID NO: 1307 - protein, 9 aa, Homo sapiens
GEHAWTHRL

SEQ ID NO: 1308 - protein, 9 aa, Homo sapiens
EEISDPEED

SEQ ID NO: 1309 - protein, 8 aa, Homo sapiens
SEKIFYVY

SEQ ID NO: 1310 - protein, 8 aa, Homo sapiens
PEKIQKAF

SEQ ID NO: 1311 - protein, 8 aa, Homo sapiens
VERPQMTF

SEQ ID NO: 1312 - protein, 8 aa, Homo sapiens
EEWEKMKA

SEQ ID NO: 1313 - protein, 8 aa, Homo sapiens
EEVPEASG

SEQ ID NO: 1314 - protein, 10 aa, Homo sapiens
GRLQGISPKI

SEQ ID NO: 1315 - protein, 10 aa, Homo sapiens
KRKYEAMTKL

SEQ ID NO: 1316 - protein, 10 aa, Homo sapiens
KRGEHAWTHR

SEQ ID NO: 1317 - protein, 10 aa, Homo sapiens
ERPQMTFGRL

SEQ ID NO: 1318 - protein, 10 aa, Homo sapiens
ERSGPKRGEH

SEQ ID NO: 1319 - protein, 9 aa, Homo sapiens
GRLQGISPK

SEQ ID NO: 1320 - protein, 9 aa, Homo sapiens
KRKYEAMTK

SEQ ID NO: 1321 - protein, 9 aa, Homo sapiens
HRLRERKQL

SEQ ID NO: 1322 - protein, 9 aa, Homo sapiens
RRPTVGAQI

SEQ ID NO: 1323 - protein, 9 aa, Homo sapiens
LRERKQLVI

SEQ ID NO: 1324 - protein, 10 aa, Homo sapiens
THRLRERKQL

SEQ ID NO: 1325 - protein, 10 aa, Homo sapiens
KRKYEAMTKL

SEQ ID NO: 1326 - protein, 10 aa, Homo sapiens
ERPQMTFGRL

SEQ ID NO: 1327 - protein, 10 aa, Homo sapiens
GRLQGISPKI

SEQ ID NO: 1328 - protein, 10 aa, Homo sapiens
IHERSGPKRG

SEQ ID NO: 1329 - protein, 9 aa, Homo sapiens
LRERKQLVI

SEQ ID NO: 1330 - protein, 9 aa, Homo sapiens
RRPTVGAQI

SEQ ID NO: 1331 - protein, 9 aa, Homo sapiens
AFDDIAKYF

SEQ ID NO: 1332 - protein, 9 aa, Homo sapiens
TKLGFKATL

SEQ ID NO: 1333 - protein, 9 aa, Homo sapiens
AEDFQGNDL

SEQ ID NO: 1334 - protein, 10 aa, Homo sapiens
KAFDDIAKYF

SEQ ID NO: 1335 - protein, 10 aa, Homo sapiens
KASEKIFYVY

SEQ ID NO: 1336 - protein, 10 aa, Homo sapiens
RAEDFQGNDL

SEQ ID NO: 1337 - protein, 10 aa, Homo sapiens
MTKLGFKATL

SEQ ID NO: 1338 - protein, 10 aa, Homo sapiens
ASEKIFYVYM

SEQ ID NO: 1339 - protein, 10 aa, Homo sapiens
IAKYFSKEEW

SEQ ID NO: 1340 - protein, 9 aa, Homo sapiens
KAFDDIAKY

SEQ ID NO: 1341 - protein, 9 aa, Homo sapiens
FSKEEWEKM

SEQ ID NO: 1342 - protein, 9 aa, Homo sapiens
ASEKIFYVY

SEQ ID NO: 1343 - protein, 9 aa, Homo sapiens
QVERPQMTF

SEQ ID NO: 1344 - protein, 15 aa, Homo sapiens
QMTFGRLQGISPKIM

SEQ ID NO: 1345 - protein, 15 aa, Homo sapiens
RPTVGAQIPEKIQKA

SEQ ID NO: 1346 - protein, 15 aa, Homo sapiens
FGRLQGISPKIMPKK

SEQ ID NO: 1347 - protein, 15 aa, Homo sapiens
DDAFARRPTVGAQIP

SEQ ID NO: 1348 - protein, 15 aa, Homo sapiens
KEEWEKMKASEKIFY

SEQ ID NO: 1349 - protein, 15 aa, Homo sapiens
KRKYEAMTKLGFKAT

SEQ ID NO: 1350 - protein, 15 aa, Homo sapiens
KLGFKATLPPFMCNK

SEQ ID NO: 1351 - protein, 15 aa, Homo sapiens
QKAFDDIAKYFSKEE

SEQ ID NO: 1352 - protein, 15 aa, Homo sapiens
PPFMCNKRAEDFQGN

SEQ ID NO: 1353 - protein, 15 aa, Homo sapiens
QGISPKIMPKKPAEE

SEQ ID NO: 1354 - protein, 15 aa, Homo sapiens
GNDLDNDPNRGNQVE

SEQ ID NO: 1355 - protein, 15 aa, Homo sapiens
GAQIPEKIQKAFDDI

SEQ ID NO: 1356 - protein, 15 aa, Homo sapiens
WEKMKASEKIFYVYM

SEQ ID NO: 1357 - protein, 15 aa, Homo sapiens
EKIFYVYMKRKYEAM

SEQ ID NO: 1358 - protein, 15 aa, Homo sapiens
KIQKAFDDIAKYFSK

SEQ ID NO: 1359 - protein, 15 aa, Homo sapiens
THRLRERKQLVIYEE

SEQ ID NO: 1360 - protein, 15 aa, Homo sapiens
IQKAFDDIAKYFSKE

SEQ ID NO: 1361 - protein, 15 aa, Homo sapiens
YVYMKRKYEAMTKLG

SEQ ID NO: 1362 - protein, 15 aa, Homo sapiens
PPFMCNKRAEDFQGN

SEQ ID NO: 1363 - protein, 15 aa, Homo sapiens
CNKRAEDFQGNDLDN

SEQ ID NO: 1364 - protein, 15 aa, Homo sapiens
GNDLDNDPNRGNQVE

SEQ ID NO: 1365 - protein, 15 aa, Homo sapiens
QKAFDDIAKYFSKEE

SEQ ID NO: 1366 - protein, 15 aa, Homo sapiens
AKYFSKEEWEKMKAS

SEQ ID NO: 1367 - protein, 15 aa, Homo sapiens
PEKIQKAFDDIAKYF

SEQ ID NO: 1368 - protein, 15 aa, Homo sapiens
SEKIFYVYMKRKYEA

SEQ ID NO: 1369 - protein, 15 aa, Homo sapiens
YEAMTKLGFKATLPP

SEQ ID NO: 1370 - protein, 15 aa, Homo sapiens
FGRLQGISPKIMPKK

SEQ ID NO: 1371 - protein, 15 aa, Homo sapiens
SEEVPEASGPQNDGK

SEQ ID NO: 1372 - protein, 15 aa, Homo sapiens
QLVIYEEISDPEEDD

SEQ ID NO: 1373 - protein, 15 aa, Homo sapiens
MNGDDAFARRPTVGA

SEQ ID NO: 1374 - protein, 15 aa, Homo sapiens
KLGFKATLPPFMCNK

SEQ ID NO: 1375 - protein, 15 aa, Homo sapiens
PEKIQKAFDDIAKYF

SEQ ID NO: 1376 - protein, 15 aa, Homo sapiens
CPPGKPTTSEKIHER

SEQ ID NO: 1377 - protein, 15 aa, Homo sapiens
DDAFARRPTVGAQIP

SEQ ID NO: 1378 - protein, 15 aa, Homo sapiens
FGRLQGISPKIMPKK

SEQ ID NO: 1379 - protein, 15 aa, Homo sapiens
PKRGEHAWTHRLRER

SEQ ID NO: 1380 - protein, 15 aa, Homo sapiens
LVIYEEISDPEEDDE

SEQ ID NO: 1381 - protein, 15 aa, Homo sapiens
WEKMKASEKIFYVYM

SEQ ID NO: 1382 - protein, 15 aa, Homo sapiens
AKYFSKEEWEKMKAS

SEQ ID NO: 1383 - protein, 15 aa, Homo sapiens
EWEKMKASEKIFYVY

SEQ ID NO: 1384 - protein, 15 aa, Homo sapiens
QKAFDDIAKYFSKEE

SEQ ID NO: 1385 - protein, 15 aa, Homo sapiens
KIFYVYMKRKYEAMT

SEQ ID NO: 1386 - protein, 15 aa, Homo sapiens
KRKYEAMTKLGFKAT

SEQ ID NO: 1387 - protein, 15 aa, Homo sapiens
QMTFGRLQGISPKIM

SEQ ID NO: 1388 - protein, 15 aa, Homo sapiens
EHAWTHRLRERKQLV

SEQ ID NO: 1389 - protein, 15 aa, Homo sapiens
EKIFYVYMKRKYEAM

SEQ ID NO: 1390 - protein, 15 aa, Homo sapiens
IFYVYMKRKYEAMTK

SEQ ID NO: 1391 - protein, 15 aa, Homo sapiens
GISPKIMPKKPAEEG

SEQ ID NO: 1392 - protein, 15 aa, Homo sapiens
LPPFMCNKRAEDFQG

SEQ ID NO: 1393 - protein, 15 aa, Homo sapiens
DDAFARRPTVGAQIP

SEQ ID NO: 1394 - protein, 15 aa, Homo sapiens
RKQLVIYEEISDPEE

SEQ ID NO: 1395 - protein, 15 aa, Homo sapiens
FDDIAKYFSKEEWEK

SEQ ID NO: 1396 - protein, 15 aa, Homo sapiens
VYMKRKYEAMTKLGF

SEQ ID NO: 1397 - protein, 15 aa, Homo sapiens
MTKLGFKATLPPFMC

SEQ ID NO: 1398 - protein, 15 aa, Homo sapiens
KATLPPFMCNKRAED

SEQ ID NO: 1399 - protein, 15 aa, Homo sapiens
DDAFARRPTVGAQIP

SEQ ID NO: 1400 - protein, 15 aa, Homo sapiens
QMTFGRLQGISPKIM

SEQ ID NO: 1401 - protein, 15 aa, Homo sapiens
EKIQKAFDDIAKYFS

SEQ ID NO: 1402 - protein, 15 aa, Homo sapiens
YFSKEEWEKMKASEK

SEQ ID NO: 1403 - protein, 15 aa, Homo sapiens
ASEKIFYVYMKRKYE

SEQ ID NO: 1404 - protein, 256 aa, Homo sapiens
MAHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 1405 - protein, 11 aa, Homo sapiens
FPDFFTRVALY

SEQ ID NO: 1406 - protein, 11 aa, Homo sapiens
GHEAQPHSRPY

SEQ ID NO: 1407 - protein, 11 aa, Homo sapiens
FSVAQVFLNNY

SEQ ID NO: 1408 - protein, 10 aa, Homo sapiens
SVAQVFLNNY

SEQ ID NO: 1409 - protein, 10 aa, Homo sapiens
HEAQPHSRPY

SEQ ID NO: 1410 - protein, 10 aa, Homo sapiens
LRDIPQRLVN

SEQ ID NO: 1411 - protein, 9 aa, Homo sapiens
DFFTRVALY

SEQ ID NO: 1412 - protein, 9 aa, Homo sapiens
EAQPHSRPY

SEQ ID NO: 1413 - protein, 9 aa, Homo sapiens
VAQVFLNNY

SEQ ID NO: 1414 - protein, 11 aa, Homo sapiens
YVDWIRSTLRR

SEQ ID NO: 1415 - protein, 10 aa, Homo sapiens
ALASVLLALL

SEQ ID NO: 1416 - protein, 10 aa, Homo sapiens
KLNDVLLIQL

SEQ ID NO: 1417 - protein, 10 aa, Homo sapiens
VLQELNVTVV

SEQ ID NO: 1418 - protein, 10 aa, Homo sapiens
LICDGIIQGI

SEQ ID NO: 1419 - protein, 10 aa, Homo sapiens
LIHPSFVLTA

SEQ ID NO: 1420 - protein, 10 aa, Homo sapiens
RLFPDFFTRV

SEQ ID NO: 1421 - protein, 10 aa, Homo sapiens
ALYVDWIRST

SEQ ID NO: 1422 - protein, 9 aa, Homo sapiens
NLSASVATV

SEQ ID NO: 1423 - protein, 9 aa, Homo sapiens
LLALLLSGA

SEQ ID NO: 1424 - protein, 9 aa, Homo sapiens
CLAMGWGRV

SEQ ID NO: 1425 - protein, 10 aa, Homo sapiens
FLNNYDAENK

SEQ ID NO: 1426 - protein, 10 aa, Homo sapiens
TLRRVEAKGR

SEQ ID NO: 1427 - protein, 10 aa, Homo sapiens
QVLQELNVTV

SEQ ID NO: 1428 - protein, 10 aa, Homo sapiens
IVGGHEAQPH

SEQ ID NO: 1429 - protein, 10 aa, Homo sapiens
RLVNVVLGAH

SEQ ID NO: 1430 - protein, 9 aa, Homo sapiens
ALLLSGAAR

SEQ ID NO: 1431 - protein, 9 aa, Homo sapiens
FVIWGCATR

SEQ ID NO: 1432 - protein, 9 aa, Homo sapiens
RLFPDFFTR

SEQ ID NO: 1433 - protein, 9 aa, Homo sapiens
VVLGAHNVR

SEQ ID NO: 1434 - protein, 9 aa, Homo sapiens
ELNVTVVTF

SEQ ID NO: 1435 - protein, 10 aa, Homo sapiens
FVLTAAHCLR

SEQ ID NO: 1436 - protein, 10 aa, Homo sapiens
NVTVVTFFCR

SEQ ID NO: 1437 - protein, 10 aa, Homo sapiens
YVDWIRSTLR

SEQ ID NO: 1438 - protein, 10 aa, Homo sapiens
RVALYVDWIR

SEQ ID NO: 1439 - protein, 9 aa, Homo sapiens
STLRRVEAK

SEQ ID NO: 1440 - protein, 9 aa, Homo sapiens
VVLGAHNVR

SEQ ID NO: 1441 - protein, 9 aa, Homo sapiens
ASVLLALLL

SEQ ID NO: 1442 - protein, 9 aa, Homo sapiens
SVLLALLLS

SEQ ID NO: 1443 - protein, 9 aa, Homo sapiens
VTVVTFFCR

SEQ ID NO: 1444 - protein, 9 aa, Homo sapiens
SVAQVFLNN

SEQ ID NO: 1445 - protein, 10 aa, Homo sapiens
LYVDWIRSTL

SEQ ID NO: 1446 - protein, 10 aa, Homo sapiens
CFGDSGGPLI

SEQ ID NO: 1447 - protein, 10 aa, Homo sapiens
TFVPRRKAGI

SEQ ID NO: 1448 - protein, 10 aa, Homo sapiens
SFVLTAAHCL

SEQ ID NO: 1449 - protein, 10 aa, Homo sapiens
PSPALASVLL

SEQ ID NO: 1450 - protein, 10 aa, Homo sapiens
VIWGCATRLF

SEQ ID NO: 1451 - protein, 9 aa, Homo sapiens
TFFCRPHNI

SEQ ID NO: 1452 - protein, 9 aa, Homo sapiens
CFGDSGGPL

SEQ ID NO: 1453 - protein, 9 aa, Homo sapiens
HFSVAQVFL

SEQ ID NO: 1454 - protein, 9 aa, Homo sapiens
NKLNDVLLI

SEQ ID NO: 1455 - protein, 10 aa, Homo sapiens
SVAQVFLNNY

SEQ ID NO: 1456 - protein, 10 aa, Homo sapiens
DGIIQGIDSF

SEQ ID NO: 1457 - protein, 10 aa, Homo sapiens
DVLLIQLSSP

SEQ ID NO: 1458 - protein, 10 aa, Homo sapiens
ELNVTVVTFF

SEQ ID NO: 1459 - protein, 10 aa, Homo sapiens
FVIWGCATRL

SEQ ID NO: 1460 - protein, 9 aa, Homo sapiens
DFFTRVALY

SEQ ID NO: 1461 - protein, 9 aa, Homo sapiens
ELNVTVVTF

SEQ ID NO: 1462 - protein, 9 aa, Homo sapiens
DVLLIQLSS

SEQ ID NO: 1463 - protein, 9 aa, Homo sapiens
YVDWIRSTL

SEQ ID NO: 1464 - protein, 9 aa, Homo sapiens
EAQPHSRPY

SEQ ID NO: 1465 - protein, 11 aa, Homo sapiens
DSFVIWGCATR

SEQ ID NO: 1466 - protein, 11 aa, Homo sapiens
LLALLLSGAAR

SEQ ID NO: 1467 - protein, 11 aa, Homo sapiens
GTQCLAMGWGR

SEQ ID NO: 1468 - protein, 11 aa, Homo sapiens
STLRRVEAKGR

SEQ ID NO: 1469 - protein, 10 aa, Homo sapiens
LALLLSGAAR

SEQ ID NO: 1470 - protein, 10 aa, Homo sapiens
YVDWIRSTLR

SEQ ID NO: 1471 - protein, 10 aa, Homo sapiens
NVVLGAHNVR

SEQ ID NO: 1472 - protein, 10 aa, Homo sapiens
NVTVVTFFCR

SEQ ID NO: 1473 - protein, 10 aa, Homo sapiens
RVALYVDWIR

SEQ ID NO: 1474 - protein, 11 aa, Homo sapiens
ATRLFPDFFTR

SEQ ID NO: 1475 - protein, 10 aa, Homo sapiens
FPDFFTRVAL

SEQ ID NO: 1476 - protein, 10 aa, Homo sapiens
PPSPALASVL

SEQ ID NO: 1477 - protein, 10 aa, Homo sapiens
IPQRLVNVVL

SEQ ID NO: 1478 - protein, 10 aa, Homo sapiens
QPVPHGTQCL

SEQ ID NO: 1479 - protein, 10 aa, Homo sapiens
VPHGTQCLAM

SEQ ID NO: 1480 - protein, 10 aa, Homo sapiens
DPPAQVLQEL

SEQ ID NO: 1481 - protein, 9 aa, Homo sapiens
SPALASVLL

SEQ ID NO: 1482 - protein, 9 aa, Homo sapiens
PPAQVLQEL

SEQ ID NO: 1483 - protein, 9 aa, Homo sapiens
AHRPPSPAL

SEQ ID NO: 1484 - protein, 9 aa, Homo sapiens
HPSFVLTAA

SEQ ID NO: 1485 - protein, 9 aa, Homo sapiens
ENKLNDVLL

SEQ ID NO: 1486 - protein, 9 aa, Homo sapiens
VPRRKAGIC

SEQ ID NO: 1487 - protein, 9 aa, Homo sapiens
CLRDIPQRL

SEQ ID NO: 1488 - protein, 9 aa, Homo sapiens
SPALASVLL

SEQ ID NO: 1489 - protein, 9 aa, Homo sapiens
PQRLVNVVL

SEQ ID NO: 1490 - protein, 9 aa, Homo sapiens
SASVATVQL

SEQ ID NO: 1491 - protein, 8 aa, Homo sapiens
VPRRKAGI

SEQ ID NO: 1492 - protein, 8 aa, Homo sapiens
ENKLNDVL

SEQ ID NO: 1493 - protein, 8 aa, Homo sapiens
VDWIRSTL

SEQ ID NO: 1494 - protein, 8 aa, Homo sapiens
DFFTRVAL

SEQ ID NO: 1495 - protein, 10 aa, Homo sapiens
QQHFSVAQVF

SEQ ID NO: 1496 - protein, 10 aa, Homo sapiens
SVAQVFLNNY

SEQ ID NO: 1497 - protein, 10 aa, Homo sapiens
ELNVTVVTFF

SEQ ID NO: 1498 - protein, 10 aa, Homo sapiens
RLFPDFFTRV

SEQ ID NO: 1499 - protein, 10 aa, Homo sapiens
RLVNVVLGAH

SEQ ID NO: 1500 - protein, 10 aa, Homo sapiens
KLNDVLLIQL

SEQ ID NO: 1501 - protein, 9 aa, Homo sapiens
ELNVTVVTF

SEQ ID NO: 1502 - protein, 9 aa, Homo sapiens
TQEPTQQHF

SEQ ID NO: 1503 - protein, 9 aa, Homo sapiens
GIIQGIDSF

SEQ ID NO: 1504 - protein, 9 aa, Homo sapiens
ALASVLLAL

SEQ ID NO: 1505 - protein, 9 aa, Homo sapiens
AENKLNDVL

SEQ ID NO: 1506 - protein, 9 aa, Homo sapiens
DFFTRVALY

SEQ ID NO: 1507 - protein, 9 aa, Homo sapiens
ATRLFPDFF

SEQ ID NO: 1508 - protein, 9 aa, Homo sapiens
GGTLIHPSF

SEQ ID NO: 1509 - protein, 8 aa, Homo sapiens
QEPTQQHF

SEQ ID NO: 1510 - protein, 8 aa, Homo sapiens
LNVTVVTF

SEQ ID NO: 1511 - protein, 8 aa, Homo sapiens
QELNVTVV

SEQ ID NO: 1512 - protein, 8 aa, Homo sapiens
NDVLLIQL

SEQ ID NO: 1513 - protein, 9 aa, Homo sapiens
PRRKAGICF

SEQ ID NO: 1514 - protein, 9 aa, Homo sapiens
CATRLFPDF

SEQ ID NO: 1515 - protein, 10 aa, Homo sapiens
TRLFPDFFTR

SEQ ID NO: 1516 - protein, 10 aa, Homo sapiens
SRPYMASLQM

SEQ ID NO: 1517 - protein, 10 aa, Homo sapiens
VRTQEPTQQH

SEQ ID NO: 1518 - protein, 10 aa, Homo sapiens
ARAAEIVGGH

SEQ ID NO: 1519 - protein, 9 aa, Homo sapiens
CRPHNICTF

SEQ ID NO: 1520 - protein, 9 aa, Homo sapiens
PRRKAGICF

SEQ ID NO: 1521 - protein, 9 aa, Homo sapiens
GIIQGIDSF

SEQ ID NO: 1522 - protein, 9 aa, Homo sapiens
MRGNPGSHF

SEQ ID NO: 1523 - protein, 9 aa, Homo sapiens
LRRVEAKGR

SEQ ID NO: 1524 - protein, 9 aa, Homo sapiens
RRVEAKGRP

SEQ ID NO: 1525 - protein, 10 aa, Homo sapiens
FPDFFTRVAL

SEQ ID NO: 1526 - protein, 10 aa, Homo sapiens
IPQRLVNVVL

SEQ ID NO: 1527 - protein, 10 aa, Homo sapiens
PPSPALASVL

SEQ ID NO: 1528 - protein, 10 aa, Homo sapiens
DPPAQVLQEL

SEQ ID NO: 1529 - protein, 10 aa, Homo sapiens
QPVPHGTQCL

SEQ ID NO: 1530 - protein, 10 aa, Homo sapiens
VPRRKAGICF

SEQ ID NO: 1531 - protein, 9 aa, Homo sapiens
SPALASVLL

SEQ ID NO: 1532 - protein, 9 aa, Homo sapiens
PPAQVLQEL

SEQ ID NO: 1533 - protein, 9 aa, Homo sapiens
GPLICDGII

SEQ ID NO: 1534 - protein, 9 aa, Homo sapiens
RPYMASLQM

SEQ ID NO: 1535 - protein, 10 aa, Homo sapiens
RTQEPTQQHF

SEQ ID NO: 1536 - protein, 10 aa, Homo sapiens
LTAAHCLRDI

SEQ ID NO: 1537 - protein, 10 aa, Homo sapiens
PSPALASVLL

SEQ ID NO: 1538 - protein, 10 aa, Homo sapiens
LSGAARAAEI

SEQ ID NO: 1539 - protein, 10 aa, Homo sapiens
GSHFCGGTLI

SEQ ID NO: 1540 - protein, 9 aa, Homo sapiens
ASVLLALLL

SEQ ID NO: 1541 - protein, 9 aa, Homo sapiens
EAQPHSRPY

SEQ ID NO: 1542 - protein, 9 aa, Homo sapiens
HSRPYMASL

SEQ ID NO: 1543 - protein, 9 aa, Homo sapiens
RVALYVDWI

SEQ ID NO: 1544 - protein, 9 aa, Homo sapiens
CATRLFPDF

SEQ ID NO: 1545 - protein, 15 aa, Homo sapiens
AMGWGRVGAHDPPAQ

SEQ ID NO: 1546 - protein, 15 aa, Homo sapiens
SVLLALLLSGAARAA

SEQ ID NO: 1547 - protein, 15 aa, Homo sapiens
NDVLLIQLSSPANLS

SEQ ID NO: 1548 - protein, 15 aa, Homo sapiens
HPSFVLTAAHCLRDI

SEQ ID NO: 1549 - protein, 15 aa, Homo sapiens
DGIIQGIDSFVIWGC

SEQ ID NO: 1550 - protein, 15 aa, Homo sapiens
GICFGDSGGPLICDG

SEQ ID NO: 1551 - protein, 15 aa, Homo sapiens
ASVLLALLLSGAARA

SEQ ID NO: 1552 - protein, 15 aa, Homo sapiens
LLALLLSGAARAAEI

SEQ ID NO: 1553 - protein, 15 aa, Homo sapiens
ARAAEIVGGHEAQPH

SEQ ID NO: 1554 - protein, 15 aa, Homo sapiens
SLQMRGNPGSHFCGG

SEQ ID NO: 1555 - protein, 15 aa, Homo sapiens
VFLNNYDAENKLNDV

SEQ ID NO: 1556 - protein, 15 aa, Homo sapiens
SPALASVLLALLLSG

SEQ ID NO: 1557 - protein, 15 aa, Homo sapiens
VLLIQLSSPANLSAS

SEQ ID NO: 1558 - protein, 15 aa, Homo sapiens
GGPLICDGIIQGIDS

SEQ ID NO: 1559 - protein, 15 aa, Homo sapiens
AAHCLRDIPQRLVNV

SEQ ID NO: 1560 - protein, 15 aa, Homo sapiens
ATRLFPDFFTRVALY

SEQ ID NO: 1561 - protein, 15 aa, Homo sapiens
SLQMRGNPGSHFCGG

SEQ ID NO: 1562 - protein, 15 aa, Homo sapiens
HFSVAQVFLNNYDAE

SEQ ID NO: 1563 - protein, 15 aa, Homo sapiens
PAQVLQELNVTVVTF

SEQ ID NO: 1564 - protein, 15 aa, Homo sapiens
RVALYVDWIRSTLRR

SEQ ID NO: 1565 - protein, 15 aa, Homo sapiens
TRLFPDFFTRVALYV

SEQ ID NO: 1566 - protein, 15 aa, Homo sapiens
SVLLALLLSGAARAA

SEQ ID NO: 1567 - protein, 15 aa, Homo sapiens
AHCLRDIPQRLVNVV

SEQ ID NO: 1568 - protein, 15 aa, Homo sapiens
VNVVLGAHNVRTQEP

SEQ ID NO: 1569 - protein, 15 aa, Homo sapiens
DVLLIQLSSPANLSA

SEQ ID NO: 1570 - protein, 15 aa, Homo sapiens
PAQVLQELNVTVVTF

SEQ ID NO: 1571 - protein, 15 aa, Homo sapiens
VTVVTFFCRPHNICT

SEQ ID NO: 1572 - protein, 15 aa, Homo sapiens
DGIIQGIDSFVIWGC

SEQ ID NO: 1573 - protein, 15 aa, Homo sapiens
QQHFSVAQVFLNNYD

SEQ ID NO: 1574 - protein, 15 aa, Homo sapiens
VTFFCRPHNICTFVP

SEQ ID NO: 1575 - protein, 15 aa, Homo sapiens
DGIIQGIDSFVIWGC

SEQ ID NO: 1576 - protein, 15 aa, Homo sapiens
HPSFVLTAAHCLRDI

SEQ ID NO: 1577 - protein, 15 aa, Homo sapiens
GICFGDSGGPLICDG

SEQ ID NO: 1578 - protein, 15 aa, Homo sapiens
YVDWIRSTLRRVEAK

SEQ ID NO: 1579 - protein, 15 aa, Homo sapiens
AHCLRDIPQRLVNVV

SEQ ID NO: 1580 - protein, 15 aa, Homo sapiens
VLLIQLSSPANLSAS

SEQ ID NO: 1581 - protein, 15 aa, Homo sapiens
PAQVLQELNVTVVTF

SEQ ID NO: 1582 - protein, 15 aa, Homo sapiens
LQELNVTVVTFFCRP

SEQ ID NO: 1583 - protein, 15 aa, Homo sapiens
CDGIIQGIDSFVIWG

SEQ ID NO: 1584 - protein, 15 aa, Homo sapiens
SFVIWGCATRLFPDF

SEQ ID NO: 1585 - protein, 15 aa, Homo sapiens
HNICTFVPRRKAGIC

SEQ ID NO: 1586 - protein, 15 aa, Homo sapiens
FVIWGCATRLFPDFF

SEQ ID NO: 1587 - protein, 15 aa, Homo sapiens
AMGWGRVGAHDPPAQ

SEQ ID NO: 1588 - protein, 15 aa, Homo sapiens
VVTFFCRPHNICTFV

SEQ ID NO: 1589 - protein, 15 aa, Homo sapiens
DWIRSTLRRVEAKGR

SEQ ID NO: 1590 - protein, 15 aa, Homo sapiens
PDFFTRVALYVDWIR

SEQ ID NO: 1591 - protein, 15 aa, Homo sapiens
ASVLLALLLSGAARA

SEQ ID NO: 1592 - protein, 15 aa, Homo sapiens
SVLLALLLSGAARAA

SEQ ID NO: 1593 - protein, 15 aa, Homo sapiens
SRPYMASLQMRGNPG

SEQ ID NO: 1594 - protein, 15 aa, Homo sapiens
LNNYDAENKLNDVLL

SEQ ID NO: 1595 - protein, 15 aa, Homo sapiens
QVFLNNYDAENKLND

SEQ ID NO: 1596 - protein, 15 aa, Homo sapiens
PHNICTFVPRRKAGI

SEQ ID NO: 1597 - protein, 15 aa, Homo sapiens
TRLFPDFFTRVALYV

SEQ ID NO: 1598 - protein, 15 aa, Homo sapiens
VNVVLGAHNVRTQEP

SEQ ID NO: 1599 - protein, 15 aa, Homo sapiens
LIQLSSPANLSASVA

SEQ ID NO: 1600 - protein, 15 aa, Homo sapiens
PAQVLQELNVTVVTF

SEQ ID NO: 1601 - protein, 15 aa, Homo sapiens
NVTVVTFFCRPHNIC

SEQ ID NO: 1602 - protein, 15 aa, Homo sapiens
VTVVTFFCRPHNICT

SEQ ID NO: 1603 - protein, 15 aa, Homo sapiens
DGIIQGIDSFVIWGC

SEQ ID NO: 1604 - protein, 15 aa, Homo sapiens
IQGIDSFVIWGCATR

SEQ ID NO: 1605 - protein, 465 aa, Homo sapiens
METGFPAIMYPGSFIGGWGEEYLSWEGPGLPDFVFQQQPVESEAMHCSNPKSGVVLATVARGPDACQILTRAPLGQDPPQRTVLGLLTANGQYRRTCGQGITRIRCYSGSENAFPPAGKKALPDCGVQEPPKQGFDIYMDELEQGDRDSCSVREGMAFEDVYEVDTGTLKSDLHFLLDFNTVSPMLVDSSLLSQSEDISSLGTDVINVTEYAEEIYQYLREAEIRHRPKAHYMKKQPDITEGMRTILVDWLVEVGEEYKLRAETLYLAVNFLDRFLSCMSVLRGKLQLVGTAAMLLASKYEEIYPPEVDEFVYITDDTYTKRQLLKMEHLLLKVLAFDLTVPTTNQFLLQYLRRQGVCVRTENLAKYVAELSLLEADPFLKYLPSLIAAAAFCLANYTVNKHFWPETLAAFTGYSLSEIVPCLSELHKAYLDIPHRPQQAIREKYKASKYLCVSLMEPPAVLLLQ

SEQ ID NO: 1606 - protein, 11 aa, Homo sapiens
VTEYAEEIYQY

SEQ ID NO: 1607 - protein, 11 aa, Homo sapiens
VREGMAFEDVY

SEQ ID NO: 1608 - protein, 11 aa, Homo sapiens
WPETLAAFTGY

SEQ ID NO: 1609 - protein, 11 aa, Homo sapiens
GTAAMLLASKY

SEQ ID NO: 1610 - protein, 10 aa, Homo sapiens
GTDVINVTEY

SEQ ID NO: 1611 - protein, 10 aa, Homo sapiens
LLEADPFLKY

SEQ ID NO: 1612 - protein, 10 aa, Homo sapiens
PTTNQFLLQY

SEQ ID NO: 1613 - protein, 10 aa, Homo sapiens
IREKYKASKY

SEQ ID NO: 1614 - protein, 9 aa, Homo sapiens
TTNQFLLQY

SEQ ID NO: 1615 - protein, 9 aa, Homo sapiens
PPEVDEFVY

SEQ ID NO: 1616 - protein, 9 aa, Homo sapiens
AAMLLASKY

SEQ ID NO: 1617 - protein, 10 aa, Homo sapiens
TILVDWLVEV

SEQ ID NO: 1618 - protein, 10 aa, Homo sapiens
GMAFEDVYEV

SEQ ID NO: 1619 - protein, 10 aa, Homo sapiens
SLMEPPAVLL

SEQ ID NO: 1620 - protein, 9 aa, Homo sapiens
NLAKYVAEL

SEQ ID NO: 1621 - protein, 9 aa, Homo sapiens
SLSEIVPCL

SEQ ID NO: 1622 - protein, 9 aa, Homo sapiens
VLRGKLQLV

SEQ ID NO: 1623 - protein, 9 aa, Homo sapiens
SLLEADPFL

SEQ ID NO: 1624 - protein, 9 aa, Homo sapiens
SLGTDVINV

SEQ ID NO: 1625 - protein, 9 aa, Homo sapiens
TLYLAVNFL

SEQ ID NO: 1626 - protein, 9 aa, Homo sapiens
KMEHLLLKV

SEQ ID NO: 1627 - protein, 10 aa, Homo sapiens
AIREKYKASK

SEQ ID NO: 1628 - protein, 10 aa, Homo sapiens
SLLEADPFLK

SEQ ID NO: 1629 - protein, 10 aa, Homo sapiens
IVPCLSELHK

SEQ ID NO: 1630 - protein, 10 aa, Homo sapiens
LLKMEHLLLK

SEQ ID NO: 1631 - protein, 10 aa, Homo sapiens
YLRRQGVCVR

SEQ ID NO: 1632 - protein, 9 aa, Homo sapiens
CVRTENLAK

SEQ ID NO: 1633 - protein, 9 aa, Homo sapiens
CLANYTVNK

SEQ ID NO: 1634 - protein, 9 aa, Homo sapiens
FVYITDDTY

SEQ ID NO: 1635 - protein, 9 aa, Homo sapiens
ALPDCGVQE

SEQ ID NO: 1636 - protein, 9 aa, Homo sapiens
FIGGWGEEY

SEQ ID NO: 1637 - protein, 9 aa, Homo sapiens
SVLRGKLQL

SEQ ID NO: 1638 - protein, 10 aa, Homo sapiens
DTYTKRQLLK

SEQ ID NO: 1639 - protein, 10 aa, Homo sapiens
GTAAMLLASK

SEQ ID NO: 1640 - protein, 10 aa, Homo sapiens
IVPCLSELHK

SEQ ID NO: 1641 - protein, 10 aa, Homo sapiens
SLLEADPFLK

SEQ ID NO: 1642 - protein, 10 aa, Homo sapiens
LLKMEHLLLK

SEQ ID NO: 1643 - protein, 9 aa, Homo sapiens
GVVLATVAR

SEQ ID NO: 1644 - protein, 9 aa, Homo sapiens
CVRTENLAK

SEQ ID NO: 1645 - protein, 9 aa, Homo sapiens
RTCGQGITR

SEQ ID NO: 1646 - protein, 9 aa, Homo sapiens
LVEVGEEYK

SEQ ID NO: 1647 - protein, 9 aa, Homo sapiens
YLAVNFLDR

SEQ ID NO: 1648 - protein, 10 aa, Homo sapiens
EYLSWEGPGL

SEQ ID NO: 1649 - protein, 10 aa, Homo sapiens
EYAEEIYQYL

SEQ ID NO: 1650 - protein, 10 aa, Homo sapiens
PFLKYLPSLI

SEQ ID NO: 1651 - protein, 10 aa, Homo sapiens
IYQYLREAEI

SEQ ID NO: 1652 - protein, 9 aa, Homo sapiens
IYPPEVDEF

SEQ ID NO: 1653 - protein, 9 aa, Homo sapiens
KYVAELSLL

SEQ ID NO: 1654 - protein, 9 aa, Homo sapiens
VYEVDTGTL

SEQ ID NO: 1655 - protein, 9 aa, Homo sapiens
HYMKKQPDI

SEQ ID NO: 1656 - protein, 9 aa, Homo sapiens
EYKLRAETL

SEQ ID NO: 1657 - protein, 9 aa, Homo sapiens
CYSGSENAF

SEQ ID NO: 1658 - protein, 10 aa, Homo sapiens
DVYEVDTGTL

SEQ ID NO: 1659 - protein, 10 aa, Homo sapiens
ETLYLAVNFL

SEQ ID NO: 1660 - protein, 10 aa, Homo sapiens
EIYPPEVDEF

SEQ ID NO: 1661 - protein, 10 aa, Homo sapiens
DPFLKYLPSL

SEQ ID NO: 1662 - protein, 9 aa, Homo sapiens
EIVPCLSEL

SEQ ID NO: 1663 - protein, 9 aa, Homo sapiens
ETGFPAIMY

SEQ ID NO: 1664 - protein, 9 aa, Homo sapiens
ETLAAFTGY

SEQ ID NO: 1665 - protein, 9 aa, Homo sapiens
EYAEEIYQY

SEQ ID NO: 1666 - protein, 9 aa, Homo sapiens
DTGTLKSDL

SEQ ID NO: 1667 - protein, 9 aa, Homo sapiens
EVDEFVYIT

SEQ ID NO: 1668 - protein, 11 aa, Homo sapiens
DVYEVDTGTLK

SEQ ID NO: 1669 - protein, 11 aa, Homo sapiens
DIPHRPQQAIR

SEQ ID NO: 1670 - protein, 10 aa, Homo sapiens
DTYTKRQLLK

SEQ ID NO: 1671 - protein, 9 aa, Homo sapiens
TAAMLLASK

SEQ ID NO: 1672 - protein, 9 aa, Homo sapiens
ITDDTYTKR

SEQ ID NO: 1673 - protein, 9 aa, Homo sapiens
GVVLATVAR

SEQ ID NO: 1674 - protein, 9 aa, Homo sapiens
EVGEEYKLR

SEQ ID NO: 1675 - protein, 9 aa, Homo sapiens
RTCGQGITR

SEQ ID NO: 1676 - protein, 9 aa, Homo sapiens
DACQILTRA

SEQ ID NO: 1677 - protein, 9 aa, Homo sapiens
KAYLDIPHR

SEQ ID NO: 1678 - protein, 10 aa, Homo sapiens
DPPQRTVLGL

SEQ ID NO: 1679 - protein, 10 aa, Homo sapiens
SPMLVDSSLL

SEQ ID NO: 1680 - protein, 10 aa, Homo sapiens
DPFLKYLPSL

SEQ ID NO: 1681 - protein, 10 aa, Homo sapiens
NPKSGVVLAT

SEQ ID NO: 1682 - protein, 10 aa, Homo sapiens
LPSLIAAAAF

SEQ ID NO: 1683 - protein, 9 aa, Homo sapiens
PPQRTVLGL

SEQ ID NO: 1684 - protein, 9 aa, Homo sapiens
FPPAGKKAL

SEQ ID NO: 1685 - protein, 9 aa, Homo sapiens
GPGLPDFVF

SEQ ID NO: 1686 - protein, 9 aa, Homo sapiens
IPHRPQQAI

SEQ ID NO: 1687 - protein, 9 aa, Homo sapiens
VPTTNQFLL

SEQ ID NO: 1688 - protein, 9 aa, Homo sapiens
EYKLRAETL

SEQ ID NO: 1689 - protein, 9 aa, Homo sapiens
LLKVLAFDL

SEQ ID NO: 1690 - protein, 9 aa, Homo sapiens
TLKSDLHFL

SEQ ID NO: 1691 - protein, 9 aa, Homo sapiens
LLKMEHLLL

SEQ ID NO: 1692 - protein, 9 aa, Homo sapiens
HLLLKVLAF

SEQ ID NO: 1693 - protein, 8 aa, Homo sapiens
VLRGKLQL

SEQ ID NO: 1694 - protein, 8 aa, Homo sapiens
FLKYLPSL

SEQ ID NO: 1695 - protein, 8 aa, Homo sapiens
NPKSGVVL

SEQ ID NO: 1696 - protein, 8 aa, Homo sapiens
PPAGKKAL

SEQ ID NO: 1697 - protein, 8 aa, Homo sapiens
LAKYVAEL

SEQ ID NO: 1698 - protein, 10 aa, Homo sapiens
CVRTENLAKY

SEQ ID NO: 1699 - protein, 10 aa, Homo sapiens
GQGITRIRCY

SEQ ID NO: 1700 - protein, 10 aa, Homo sapiens
LLASKYEEIY

SEQ ID NO: 1701 - protein, 10 aa, Homo sapiens
LLEADPFLKY

SEQ ID NO: 1702 - protein, 9 aa, Homo sapiens
WLVEVGEEY

SEQ ID NO: 1703 - protein, 9 aa, Homo sapiens
PQQAIREKY

SEQ ID NO: 1704 - protein, 9 aa, Homo sapiens
CLSELHKAY

SEQ ID NO: 1705 - protein, 9 aa, Homo sapiens
GLLTANGQY

SEQ ID NO: 1706 - protein, 9 aa, Homo sapiens
HLLLKVLAF

SEQ ID NO: 1707 - protein, 9 aa, Homo sapiens
VQEPPKQGF

SEQ ID NO: 1708 - protein, 9 aa, Homo sapiens
MEHLLLKVL

SEQ ID NO: 1709 - protein, 9 aa, Homo sapiens
WEGPGLPDF

SEQ ID NO: 1710 - protein, 9 aa, Homo sapiens
LEADPFLKY

SEQ ID NO: 1711 - protein, 9 aa, Homo sapiens
REKYKASKY

SEQ ID NO: 1712 - protein, 8 aa, Homo sapiens
PEVDEFVY

SEQ ID NO: 1713 - protein, 8 aa, Homo sapiens
YEVDTGTL

SEQ ID NO: 1714 - protein, 8 aa, Homo sapiens
QEPPKQGF

SEQ ID NO: 1715 - protein, 8 aa, Homo sapiens
TEYAEEIY

SEQ ID NO: 1716 - protein, 8 aa, Homo sapiens
AEEIYQYL

SEQ ID NO: 1717 - protein, 8 aa, Homo sapiens
TEGMRTIL

SEQ ID NO: 1718 - protein, 10 aa, Homo sapiens
RRTCGQGITR

SEQ ID NO: 1719 - protein, 10 aa, Homo sapiens
DRFLSCMSVL

SEQ ID NO: 1720 - protein, 10 aa, Homo sapiens
HRPQQAIREK

SEQ ID NO: 1721 - protein, 10 aa, Homo sapiens
HRPKAHYMKK

SEQ ID NO: 1722 - protein, 10 aa, Homo sapiens
IREKYKASKY

SEQ ID NO: 1723 - protein, 10 aa, Homo sapiens
KRQLLKMEHL

SEQ ID NO: 1724 - protein, 9 aa, Homo sapiens
LRRQGVCVR

SEQ ID NO: 1725 - protein, 9 aa, Homo sapiens
IREKYKASK

SEQ ID NO: 1726 - protein, 9 aa, Homo sapiens
KRQLLKMEH

SEQ ID NO: 1727 - protein, 9 aa, Homo sapiens
LREAEIRHR

SEQ ID NO: 1728 - protein, 10 aa, Homo sapiens
PPQRTVLGLL

SEQ ID NO: 1729 - protein, 10 aa, Homo sapiens
EPPKQGFDIY

SEQ ID NO: 1730 - protein, 10 aa, Homo sapiens
SPMLVDSSLL

SEQ ID NO: 1731 - protein, 10 aa, Homo sapiens
DPFLKYLPSL

SEQ ID NO: 1732 - protein, 10 aa, Homo sapiens
LPSLIAAAAF

SEQ ID NO: 1733 - protein, 9 aa, Homo sapiens
FPPAGKKAL

SEQ ID NO: 1734 - protein, 10 aa, Homo sapiens
DPPQRTVLGL

SEQ ID NO: 1735 - protein, 10 aa, Homo sapiens
RPQQAIREKY

SEQ ID NO: 1736 - protein, 10 aa, Homo sapiens
FPAIMYPGSF

SEQ ID NO: 1737 - protein, 10 aa, Homo sapiens
KSDLHFLLDF

SEQ ID NO: 1738 - protein, 10 aa, Homo sapiens
KASKYLCVSL

SEQ ID NO: 1739 - protein, 10 aa, Homo sapiens
RTCGQGITRI

SEQ ID NO: 1740 - protein, 10 aa, Homo sapiens
LAVNFLDRFL

SEQ ID NO: 1741 - protein, 10 aa, Homo sapiens
YSLSEIVPCL

SEQ ID NO: 1742 - protein, 10 aa, Homo sapiens
CSNPKSGVVL

SEQ ID NO: 1743 - protein, 10 aa, Homo sapiens
SSLLSQSEDI

SEQ ID NO: 1744 - protein, 10 aa, Homo sapiens
VSLMEPPAVL

SEQ ID NO: 1745 - protein, 9 aa, Homo sapiens
LSLLEADPF

SEQ ID NO: 1746 - protein, 9 aa, Homo sapiens
PSLIAAAAF

SEQ ID NO: 1747 - protein, 15 aa, Homo sapiens
VAELSLLEADPFLKY

SEQ ID NO: 1748 - protein, 15 aa, Homo sapiens
EHLLLKVLAFDLTVP

SEQ ID NO: 1749 - protein, 15 aa, Homo sapiens
LKYLPSLIAAAAFCL

SEQ ID NO: 1750 - protein, 15 aa, Homo sapiens
LLDFNTVSPMLVDSS

SEQ ID NO: 1751 - protein, 15 aa, Homo sapiens
SEDISSLGTDVINVT

SEQ ID NO: 1752 - protein, 15 aa, Homo sapiens
DPFLKYLPSLIAAAA

SEQ ID NO: 1753 - protein, 15 aa, Homo sapiens
NGQYRRTCGQGITRI

SEQ ID NO: 1754 - protein, 15 aa, Homo sapiens
RGKLQLVGTAAMLLA

SEQ ID NO: 1755 - protein, 15 aa, Homo sapiens
ASKYEEIYPPEVDEF

SEQ ID NO: 1756 - protein, 15 aa, Homo sapiens
YLCVSLMEPPAVLLL

SEQ ID NO: 1757 - protein, 15 aa, Homo sapiens
TGTLKSDLHFLLDFN

SEQ ID NO: 1758 - protein, 15 aa, Homo sapiens
LSLLEADPFLKYLPS

SEQ ID NO: 1759 - protein, 15 aa, Homo sapiens
DCGVQEPPKQGFDIY

SEQ ID NO: 1760 - protein, 15 aa, Homo sapiens
DLHFLLDFNTVSPML

SEQ ID NO: 1761 - protein, 15 aa, Homo sapiens
EAEIRHRPKAHYMKK

SEQ ID NO: 1762 - protein, 15 aa, Homo sapiens
VINVTEYAEEIYQYL

SEQ ID NO: 1763 - protein, 15 aa, Homo sapiens
QPDITEGMRTILVDW

SEQ ID NO: 1764 - protein, 15 aa, Homo sapiens
DWLVEVGEEYKLRAE

SEQ ID NO: 1765 - protein, 15 aa, Homo sapiens
VPCLSELHKAYLDIP

SEQ ID NO: 1766 - protein, 15 aa, Homo sapiens
GSFIGGWGEEYLSWE

SEQ ID NO: 1767 - protein, 15 aa, Homo sapiens
DLHFLLDFNTVSPML

SEQ ID NO: 1768 - protein, 15 aa, Homo sapiens
EFVYITDDTYTKRQL

SEQ ID NO: 1769 - protein, 15 aa, Homo sapiens
HKAYLDIPHRPQQAI

SEQ ID NO: 1770 - protein, 15 aa, Homo sapiens
FEDVYEVDTGTLKSD

SEQ ID NO: 1771 - protein, 15 aa, Homo sapiens
TGTLKSDLHFLLDFN

SEQ ID NO: 1772 - protein, 15 aa, Homo sapiens
VSPMLVDSSLLSQSE

SEQ ID NO: 1773 - protein, 15 aa, Homo sapiens
SEDISSLGTDVINVT

SEQ ID NO: 1774 - protein, 15 aa, Homo sapiens
YLAVNFLDRFLSCMS

SEQ ID NO: 1775 - protein, 15 aa, Homo sapiens
VNFLDRFLSCMSVLR

SEQ ID NO: 1776 - protein, 15 aa, Homo sapiens
RGKLQLVGTAAMLLA

SEQ ID NO: 1777 - protein, 15 aa, Homo sapiens
VDEFVYITDDTYTKR

SEQ ID NO: 1778 - protein, 15 aa, Homo sapiens
NGQYRRTCGQGITRI

SEQ ID NO: 1779 - protein, 15 aa, Homo sapiens
ENAFPPAGKKALPDC

SEQ ID NO: 1780 - protein, 15 aa, Homo sapiens
FEDVYEVDTGTLKSD

SEQ ID NO: 1781 - protein, 15 aa, Homo sapiens
FLLDFNTVSPMLVDS

SEQ ID NO: 1782 - protein, 15 aa, Homo sapiens
SEDISSLGTDVINVT

SEQ ID NO: 1783 - protein, 15 aa, Homo sapiens
EEIYQYLREAEIRHR

SEQ ID NO: 1784 - protein, 15 aa, Homo sapiens
RGKLQLVGTAAMLLA

SEQ ID NO: 1785 - protein, 15 aa, Homo sapiens
KLQLVGTAAMLLASK

SEQ ID NO: 1786 - protein, 15 aa, Homo sapiens
ASKYEEIYPPEVDEF

SEQ ID NO: 1787 - protein, 15 aa, Homo sapiens
YLAVNFLDRFLSCMS

SEQ ID NO: 1788 - protein, 15 aa, Homo sapiens
LANYTVNKHFWPETL

SEQ ID NO: 1789 - protein, 15 aa, Homo sapiens
HKAYLDIPHRPQQAI

SEQ ID NO: 1790 - protein, 15 aa, Homo sapiens
ETGFPAIMYPGSFIG

SEQ ID NO: 1791 - protein, 15 aa, Homo sapiens
LVEVGEEYKLRAETL

SEQ ID NO: 1792 - protein, 15 aa, Homo sapiens
ASKYLCVSLMEPPAV

SEQ ID NO: 1793 - protein, 15 aa, Homo sapiens
KRQLLKMEHLLLKVL

SEQ ID NO: 1794 - protein, 15 aa, Homo sapiens
LKMEHLLLKVLAFDL

SEQ ID NO: 1795 - protein, 15 aa, Homo sapiens
NQFLLQYLRRQGVCV

SEQ ID NO: 1796 - protein, 15 aa, Homo sapiens
CVRTENLAKYVAELS

SEQ ID NO: 1797 - protein, 15 aa, Homo sapiens
YLAVNFLDRFLSCMS

SEQ ID NO: 1798 - protein, 15 aa, Homo sapiens
LANYTVNKHFWPETL

SEQ ID NO: 1799 - protein, 15 aa, Homo sapiens
HKAYLDIPHRPQQAI

SEQ ID NO: 1800 - protein, 15 aa, Homo sapiens
ETGFPAIMYPGSFIG

SEQ ID NO: 1801 - protein, 15 aa, Homo sapiens
LVEVGEEYKLRAETL

SEQ ID NO: 1802 - protein, 15 aa, Homo sapiens
ASKYLCVSLMEPPAV

SEQ ID NO: 1803 - protein, 15 aa, Homo sapiens
KRQLLKMEHLLLKVL

SEQ ID NO: 1804 - protein, 15 aa, Homo sapiens
LKMEHLLLKVLAFDL

SEQ ID NO: 1805 - protein, 15 aa, Homo sapiens
NQFLLQYLRRQGVCV

SEQ ID NO: 1806 - protein, 15 aa, Homo sapiens
CVRTENLAKYVAELS

SEQ ID NO: 1807 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 1808 - protein, 11 aa, Homo sapiens
FVRGGCASGLY

SEQ ID NO: 1809 - protein, 11 aa, Homo sapiens
FAVQRIFENGY

SEQ ID NO: 1810 - protein, 10 aa, Homo sapiens
AVQRIFENGY

SEQ ID NO: 1811 - protein, 10 aa, Homo sapiens
GYDPVNLLND

SEQ ID NO: 1812 - protein, 10 aa, Homo sapiens
VRGGCASGLY

SEQ ID NO: 1813 - protein, 10 aa, Homo sapiens
ASEIVGGRRA

SEQ ID NO: 1814 - protein, 10 aa, Homo sapiens
FGDSGSPLVC

SEQ ID NO: 1815 - protein, 9 aa, Homo sapiens
VQRIFENGY

SEQ ID NO: 1816 - protein, 9 aa, Homo sapiens
RGGCASGLY

SEQ ID NO: 1817 - protein, 9 aa, Homo sapiens
LNDIVILQL

SEQ ID NO: 1818 - protein, 10 aa, Homo sapiens
LLNDIVILQL

SEQ ID NO: 1819 - protein, 10 aa, Homo sapiens
RLACLFLACV

SEQ ID NO: 1820 - protein, 10 aa, Homo sapiens
GLYPDAFAPV

SEQ ID NO: 1821 - protein, 9 aa, Homo sapiens
FLACVLPAL

SEQ ID NO: 1822 - protein, 9 aa, Homo sapiens
NLLNDIVIL

SEQ ID NO: 1823 - protein, 9 aa, Homo sapiens
VLQELNVTV

SEQ ID NO: 1824 - protein, 9 aa, Homo sapiens
ALLLGGTAL

SEQ ID NO: 1825 - protein, 9 aa, Homo sapiens
GIASVLQEL

SEQ ID NO: 1826 - protein, 9 aa, Homo sapiens
TLGRRLACL

SEQ ID NO: 1827 - protein, 9 aa, Homo sapiens
LLLGGTALA

SEQ ID NO: 1828 - protein, 10 aa, Homo sapiens
VVLGAHNLSR

SEQ ID NO: 1829 - protein, 10 aa, Homo sapiens
LVRGRQAGVC

SEQ ID NO: 1830 - protein, 10 aa, Homo sapiens
AVQRIFENGY

SEQ ID NO: 1831 - protein, 9 aa, Homo sapiens
NVRAVRVVL

SEQ ID NO: 1832 - protein, 9 aa, Homo sapiens
GLIHGIASF

SEQ ID NO: 1833 - protein, 9 aa, Homo sapiens
ALLLGGTAL

SEQ ID NO: 1834 - protein, 9 aa, Homo sapiens
AVRVVLGAH

SEQ ID NO: 1835 - protein, 9 aa, Homo sapiens
NLSRREPTR

SEQ ID NO: 1836 - protein, 9 aa, Homo sapiens
SLQLRGGHF

SEQ ID NO: 1837 - protein, 9 aa, Homo sapiens
FVRGGCASG

SEQ ID NO: 1838 - protein, 10 aa, Homo sapiens
VVLGAHNLSR

SEQ ID NO: 1839 - protein, 10 aa, Homo sapiens
PTRQVFAVQR

SEQ ID NO: 1840 - protein, 10 aa, Homo sapiens
NVTVVTSLCR

SEQ ID NO: 1841 - protein, 10 aa, Homo sapiens
ALASEIVGGR

SEQ ID NO: 1842 - protein, 10 aa, Homo sapiens
QVAQLPAQGR

SEQ ID NO: 1843 - protein, 9 aa, Homo sapiens
RSNVCTLVR

SEQ ID NO: 1844 - protein, 9 aa, Homo sapiens
VTVVTSLCR

SEQ ID NO: 1845 - protein, 9 aa, Homo sapiens
ASEIVGGRR

SEQ ID NO: 1846 - protein, 9 aa, Homo sapiens
AMGWGLLGR

SEQ ID NO: 1847 - protein, 9 aa, Homo sapiens
NVCTLVRGR

SEQ ID NO: 1848 - protein, 10 aa, Homo sapiens
LFLACVLPAL

SEQ ID NO: 1849 - protein, 10 aa, Homo sapiens
VSLQLRGGHF

SEQ ID NO: 1850 - protein, 10 aa, Homo sapiens
VNLLNDIVIL

SEQ ID NO: 1851 - protein, 10 aa, Homo sapiens
QCLAMGWGLL

SEQ ID NO: 1852 - protein, 10 aa, Homo sapiens
GSPLVCNGLI

SEQ ID NO: 1853 - protein, 9 aa, Homo sapiens
CFGDSGSPL

SEQ ID NO: 1854 - protein, 9 aa, Homo sapiens
QFVNWIDSI

SEQ ID NO: 1855 - protein, 9 aa, Homo sapiens
NGYDPVNLL

SEQ ID NO: 1856 - protein, 9 aa, Homo sapiens
GYDPVNLLN

SEQ ID NO: 1857 - protein, 9 aa, Homo sapiens
LYPDAFAPV

SEQ ID NO: 1858 - protein, 10 aa, Homo sapiens
ELNVTVVTSL

SEQ ID NO: 1859 - protein, 10 aa, Homo sapiens
ENGYDPVNLL

SEQ ID NO: 1860 - protein, 10 aa, Homo sapiens
MTLGRRLACL

SEQ ID NO: 1861 - protein, 10 aa, Homo sapiens
AVQRIFENGY

SEQ ID NO: 1862 - protein, 10 aa, Homo sapiens
DIVILQLNGS

SEQ ID NO: 1863 - protein, 10 aa, Homo sapiens
FVRGGCASGL

SEQ ID NO: 1864 - protein, 9 aa, Homo sapiens
DAFAPVAQF

SEQ ID NO: 1865 - protein, 9 aa, Homo sapiens
RVVLGAHNL

SEQ ID NO: 1866 - protein, 9 aa, Homo sapiens
QVFAVQRIF

SEQ ID NO: 1867 - protein, 9 aa, Homo sapiens
EIVGGRRAR

SEQ ID NO: 1868 - protein, 11 aa, Homo sapiens
CVANVNVRAVR

SEQ ID NO: 1869 - protein, 11 aa, Homo sapiens
FVNWIDSIIQR

SEQ ID NO: 1870 - protein, 11 aa, Homo sapiens
QVAQLPAQGRR

SEQ ID NO: 1871 - protein, 10 aa, Homo sapiens
PTRQVFAVQR

SEQ ID NO: 1872 - protein, 10 aa, Homo sapiens
VANVNVRAVR

SEQ ID NO: 1873 - protein, 9 aa, Homo sapiens
TVVTSLCRR

SEQ ID NO: 1874 - protein, 9 aa, Homo sapiens
LASEIVGGR

SEQ ID NO: 1875 - protein, 9 aa, Homo sapiens
NVCTLVRGR

SEQ ID NO: 1876 - protein, 9 aa, Homo sapiens
NWIDSIIQR

SEQ ID NO: 1877 - protein, 9 aa, Homo sapiens
EIVGGRRAR

SEQ ID NO: 1878 - protein, 10 aa, Homo sapiens
APVAQFVNWI

SEQ ID NO: 1879 - protein, 10 aa, Homo sapiens
CPHPRDPDPA

SEQ ID NO: 1880 - protein, 10 aa, Homo sapiens
LPALLLGGTA

SEQ ID NO: 1881 - protein, 9 aa, Homo sapiens
WPFMVSLQL

SEQ ID NO: 1882 - protein, 9 aa, Homo sapiens
YPDAFAPVA

SEQ ID NO: 1883 - protein, 9 aa, Homo sapiens
APNFVMSAA

SEQ ID NO: 1884 - protein, 9 aa, Homo sapiens
EPTRQVFAV

SEQ ID NO: 1885 - protein, 9 aa, Homo sapiens
RPHAWPFMV

SEQ ID NO: 1886 - protein, 9 aa, Homo sapiens
ACVLPALLL

SEQ ID NO: 1887 - protein, 9 aa, Homo sapiens
NVRAVRVVL

SEQ ID NO: 1888 - protein, 9 aa, Homo sapiens
TLGRRLACL

SEQ ID NO: 1889 - protein, 9 aa, Homo sapiens
SLQLRGGHF

SEQ ID NO: 1890 - protein, 9 aa, Homo sapiens
GLLGRNRGI

SEQ ID NO: 1891 - protein, 9 aa, Homo sapiens
GRRLACLFL

SEQ ID NO: 1892 - protein, 9 aa, Homo sapiens
FLACVLPAL

SEQ ID NO: 1893 - protein, 9 aa, Homo sapiens
ALLLGGTAL

SEQ ID NO: 1894 - protein, 9 aa, Homo sapiens
NLLNDIVIL

SEQ ID NO: 1895 - protein, 8 aa, Homo sapiens
VRAVRVVL

SEQ ID NO: 1896 - protein, 8 aa, Homo sapiens
SPLVCNGL

SEQ ID NO: 1897 - protein, 8 aa, Homo sapiens
CLFLACVL

SEQ ID NO: 1898 - protein, 10 aa, Homo sapiens
RQVFAVQRIF

SEQ ID NO: 1899 - protein, 10 aa, Homo sapiens
TLGRRLACLF

SEQ ID NO: 1900 - protein, 10 aa, Homo sapiens
AVQRIFENGY

SEQ ID NO: 1901 - protein, 10 aa, Homo sapiens
ALLLGGTALA

SEQ ID NO: 1902 - protein, 10 aa, Homo sapiens
ILQLNGSATI

SEQ ID NO: 1903 - protein, 10 aa, Homo sapiens
RLGNGVQCLA

SEQ ID NO: 1904 - protein, 9 aa, Homo sapiens
VQRIFENGY

SEQ ID NO: 1905 - protein, 9 aa, Homo sapiens
GLIHGIASF

SEQ ID NO: 1906 - protein, 9 aa, Homo sapiens
SLQLRGGHF

SEQ ID NO: 1907 - protein, 9 aa, Homo sapiens
QVFAVQRIF

SEQ ID NO: 1908 - protein, 9 aa, Homo sapiens
DAFAPVAQF

SEQ ID NO: 1909 - protein, 9 aa, Homo sapiens
LGRRLACLF

SEQ ID NO: 1910 - protein, 9 aa, Homo sapiens
NVRAVRVVL

SEQ ID NO: 1911 - protein, 9 aa, Homo sapiens
ASGLYPDAF

SEQ ID NO: 1912 - protein, 9 aa, Homo sapiens
ACLFLACVL

SEQ ID NO: 1913 - protein, 9 aa, Homo sapiens
GATLIAPNF

SEQ ID NO: 1914 - protein, 8 aa, Homo sapiens
REPTRQVF

SEQ ID NO: 1915 - protein, 8 aa, Homo sapiens
QELNVTVV

SEQ ID NO: 1916 - protein, 8 aa, Homo sapiens
ANVQVAQL

SEQ ID NO: 1917 - protein, 8 aa, Homo sapiens
ACVLPALL

SEQ ID NO: 1918 - protein, 10 aa, Homo sapiens
RRLGNGVQCL

SEQ ID NO: 1919 - protein, 10 aa, Homo sapiens
RRARPHAWPF

SEQ ID NO: 1920 - protein, 10 aa, Homo sapiens
GRNRGIASVL

SEQ ID NO: 1921 - protein, 10 aa, Homo sapiens
RRSNVCTLVR

SEQ ID NO: 1922 - protein, 10 aa, Homo sapiens
VRVVLGAHNL

SEQ ID NO: 1923 - protein, 9 aa, Homo sapiens
RREPTRQVF

SEQ ID NO: 1924 - protein, 9 aa, Homo sapiens
GRRLACLFL

SEQ ID NO: 1925 - protein, 9 aa, Homo sapiens
VRGGCASGL

SEQ ID NO: 1926 - protein, 9 aa, Homo sapiens
TRQVFAVQR

SEQ ID NO: 1927 - protein, 9 aa, Homo sapiens
PRDPDPASR

SEQ ID NO: 1928 - protein, 10 aa, Homo sapiens
APVAQFVNWI

SEQ ID NO: 1929 - protein, 10 aa, Homo sapiens
LFLACVLPAL

SEQ ID NO: 1930 - protein, 10 aa, Homo sapiens
VNVRAVRVVL

SEQ ID NO: 1931 - protein, 10 aa, Homo sapiens
YPDAFAPVAQ

SEQ ID NO: 1932 - protein, 9 aa, Homo sapiens
WPFMVSLQL

SEQ ID NO: 1933 - protein, 9 aa, Homo sapiens
DPVNLLNDI

SEQ ID NO: 1934 - protein, 9 aa, Homo sapiens
SPLVCNGLI

SEQ ID NO: 1935 - protein, 9 aa, Homo sapiens
ACLFLACVL

SEQ ID NO: 1936 - protein, 9 aa, Homo sapiens
NLLNDIVIL

SEQ ID NO: 1937 - protein, 9 aa, Homo sapiens
RNRGIASVL

SEQ ID NO: 1938 - protein, 10 aa, Homo sapiens
RARPHAWPFM

SEQ ID NO: 1939 - protein, 10 aa, Homo sapiens
VSLQLRGGHF

SEQ ID NO: 1940 - protein, 10 aa, Homo sapiens
GSPLVCNGLI

SEQ ID NO: 1941 - protein, 10 aa, Homo sapiens
LACVLPALLL

SEQ ID NO: 1942 - protein, 9 aa, Homo sapiens
RVVLGAHNL

SEQ ID NO: 1943 - protein, 9 aa, Homo sapiens
QVFAVQRIF

SEQ ID NO: 1944 - protein, 9 aa, Homo sapiens
GATLIAPNF

SEQ ID NO: 1945 - protein, 9 aa, Homo sapiens
ASGLYPDAF

SEQ ID NO: 1946 - protein, 9 aa, Homo sapiens
GATLIAPNF

SEQ ID NO: 1947 - protein, 9 aa, Homo sapiens
ASGLYPDAF

SEQ ID NO: 1948 - protein, 15 aa, Homo sapiens
PHAWPFMVSLQLRGG

SEQ ID NO: 1949 - protein, 15 aa, Homo sapiens
LNDIVILQLNGSATI

SEQ ID NO: 1950 - protein, 15 aa, Homo sapiens
CLFLACVLPALLLGG

SEQ ID NO: 1951 - protein, 15 aa, Homo sapiens
APNFVMSAAHCVANV

SEQ ID NO: 1952 - protein, 15 aa, Homo sapiens
ASFVRGGCASGLYPD

SEQ ID NO: 1953 - protein, 15 aa, Homo sapiens
VLPALLLGGTALASE

SEQ ID NO: 1954 - protein, 15 aa, Homo sapiens
ALASEIVGGRRARPH

SEQ ID NO: 1955 - protein, 15 aa, Homo sapiens
RAVRVVLGAHNLSRR

SEQ ID NO: 1956 - protein, 15 aa, Homo sapiens
YDPVNLLNDIVILQL

SEQ ID NO: 1957 - protein, 15 aa, Homo sapiens
NVQVAQLPAQGRRLG

SEQ ID NO: 1958 - protein, 15 aa, Homo sapiens
VFAVQRIFENGYDPV

SEQ ID NO: 1959 - protein, 15 aa, Homo sapiens
PVNLLNDIVILQLNG

SEQ ID NO: 1960 - protein, 15 aa, Homo sapiens
ASGLYPDAFAPVAQF

SEQ ID NO: 1961 - protein, 15 aa, Homo sapiens
CLFLACVLPALLLGG

SEQ ID NO: 1962 - protein, 15 aa, Homo sapiens
IFENGYDPVNLLNDI

SEQ ID NO: 1963 - protein, 15 aa, Homo sapiens
IASVLQELNVTVVTS

SEQ ID NO: 1964 - protein, 15 aa, Homo sapiens
ACLFLACVLPALLLG

SEQ ID NO: 1965 - protein, 15 aa, Homo sapiens
VRVVLGAHNLSRREP

SEQ ID NO: 1966 - protein, 15 aa, Homo sapiens
VNLLNDIVILQLNGS

SEQ ID NO: 1967 - protein, 15 aa, Homo sapiens
NDIVILQLNGSATIN

SEQ ID NO: 1968 - protein, 15 aa, Homo sapiens
PDAFAPVAQFVNWID

SEQ ID NO: 1969 - protein, 15 aa, Homo sapiens
PNFVMSAAHCVANVN

SEQ ID NO: 1970 - protein, 15 aa, Homo sapiens
NRGIASVLQELNVTV

SEQ ID NO: 1971 - protein, 15 aa, Homo sapiens
IASVLQELNVTVVTS

SEQ ID NO: 1972 - protein, 15 aa, Homo sapiens
VTVVTSLCRRSNVCT

SEQ ID NO: 1973 - protein, 15 aa, Homo sapiens
RSNVCTLVRGRQAGV

SEQ ID NO: 1974 - protein, 15 aa, Homo sapiens
AQFVNWIDSIIQRSE

SEQ ID NO: 1975 - protein, 15 aa, Homo sapiens
PHAWPFMVSLQLRGG

SEQ ID NO: 1976 - protein, 15 aa, Homo sapiens
AMGWGLLGRNRGIAS

SEQ ID NO: 1977 - protein, 15 aa, Homo sapiens
GVCFGDSGSPLVCNG

SEQ ID NO: 1978 - protein, 15 aa, Homo sapiens
GVCFGDSGSPLVCNG

SEQ ID NO: 1979 - protein, 15 aa, Homo sapiens
PHAWPFMVSLQLRGG

SEQ ID NO: 1980 - protein, 15 aa, Homo sapiens
VFAVQRIFENGYDPV

SEQ ID NO: 1981 - protein, 15 aa, Homo sapiens
ILQLNGSATINANVQ

SEQ ID NO: 1982 - protein, 15 aa, Homo sapiens
APNFVMSAAHCVANV

SEQ ID NO: 1983 - protein, 15 aa, Homo sapiens
IVILQLNGSATINAN

SEQ ID NO: 1984 - protein, 15 aa, Homo sapiens
LQELNVTVVTSLCRR

SEQ ID NO: 1985 - protein, 15 aa, Homo sapiens
VAQFVNWIDSIIQRS

SEQ ID NO: 1986 - protein, 15 aa, Homo sapiens
YDPVNLLNDIVILQL

SEQ ID NO: 1987 - protein, 15 aa, Homo sapiens
VNLLNDIVILQLNGS

SEQ ID NO: 1988 - protein, 15 aa, Homo sapiens
AMGWGLLGRNRGIAS

SEQ ID NO: 1989 - protein, 15 aa, Homo sapiens
RSNVCTLVRGRQAGV

SEQ ID NO: 1990 - protein, 15 aa, Homo sapiens
VTVVTSLCRRSNVCT

SEQ ID NO: 1991 - protein, 15 aa, Homo sapiens
ASEIVGGRRARPHAW

SEQ ID NO: 1992 - protein, 15 aa, Homo sapiens
VAQLPAQGRRLGNGV

SEQ ID NO: 1993 - protein, 15 aa, Homo sapiens
PNFVMSAAHCVANVN

SEQ ID NO: 1994 - protein, 15 aa, Homo sapiens
AVRVVLGAHNLSRRE

SEQ ID NO: 1995 - protein, 15 aa, Homo sapiens
VLGAHNLSRREPTRQ

SEQ ID NO: 1996 - protein, 15 aa, Homo sapiens
TRQVFAVQRIFENGY

SEQ ID NO: 1997 - protein, 15 aa, Homo sapiens
GWGLLGRNRGIASVL

SEQ ID NO: 1998 - protein, 15 aa, Homo sapiens
PALLLGGTALASEIV

SEQ ID NO: 1999 - protein, 15 aa, Homo sapiens
VRVVLGAHNLSRREP

SEQ ID NO: 2000 - protein, 15 aa, Homo sapiens
RIFENGYDPVNLLND

SEQ ID NO: 2001 - protein, 15 aa, Homo sapiens
ILQLNGSATINANVQ

SEQ ID NO: 2002 - protein, 15 aa, Homo sapiens
VQCLAMGWGLLGRNR

SEQ ID NO: 2003 - protein, 15 aa, Homo sapiens
WGLLGRNRGIASVLQ

SEQ ID NO: 2004 - protein, 15 aa, Homo sapiens
IASVLQELNVTVVTS

SEQ ID NO: 2005 - protein, 15 aa, Homo sapiens
ELNVTVVTSLCRRSN

SEQ ID NO: 2006 - protein, 15 aa, Homo sapiens
VTSLCRRSNVCTLVR

SEQ ID NO: 2007 - protein, 15 aa, Homo sapiens
NGLIHGIASFVRGGC

SEQ ID NO: 2008 - protein, 386 aa, Epstein-Barr virus
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 2009 - protein, 9 aa, Epstein-Barr virus
LLALLFWLY

SEQ ID NO: 2010 - protein, 10 aa, Epstein-Barr virus
WTGGALLVLY

SEQ ID NO: 2011 - protein, 11 aa, Epstein-Barr virus
LLLLALLFWLY

SEQ ID NO: 2012 - protein, 11 aa, Epstein-Barr virus
MSDWTGGALLV

SEQ ID NO: 2013 - protein, 11 aa, Epstein-Barr virus
DWTGGALLVLY

SEQ ID NO: 2014 - protein, 9 aa, Epstein-Barr virus
ALLLLLLAL

SEQ ID NO: 2015 - protein, 9 aa, Epstein-Barr virus
LLLLLLALL

SEQ ID NO: 2016 - protein, 9 aa, Epstein-Barr virus
YLLEMLWRL

SEQ ID NO: 2017 - protein, 9 aa, Epstein-Barr virus
GLALLLLLL

SEQ ID NO: 2018 - protein, 9 aa, Epstein-Barr virus
LLLALLFWL

SEQ ID NO: 2019 - protein, 9 aa, Epstein-Barr virus
ALFLGIVLF

SEQ ID NO: 2020 - protein, 9 aa, Epstein-Barr virus
QLLAFFLAF

SEQ ID NO: 2021 - protein, 9 aa, Epstein-Barr virus
LLLLLALLF

SEQ ID NO: 2022 - protein, 9 aa, Epstein-Barr virus
MLWRLGATI

SEQ ID NO: 2023 - protein, 9 aa, Epstein-Barr virus
QLTEEVENK

SEQ ID NO: 2024 - protein, 9 aa, Epstein-Barr virus
SSLGLALLL

SEQ ID NO: 2025 - protein, 9 aa, Epstein-Barr virus
IILIIFIFR

SEQ ID NO: 2026 - protein, 10 aa, Epstein-Barr virus
SSSLGLALLL

SEQ ID NO: 2027 - protein, 10 aa, Epstein-Barr virus
IIILIIFIFR

SEQ ID NO: 2028 - protein, 10 aa, Epstein-Barr virus
ESDSNSNEGR

SEQ ID NO: 2029 - protein, 9 aa, Epstein-Barr virus
LYSFALMLI

SEQ ID NO: 2030 - protein, 9 aa, Epstein-Barr virus
FFLDLILLI

SEQ ID NO: 2031 - protein, 9 aa, Epstein-Barr virus
IFIFRRDLL

SEQ ID NO: 2032 - protein, 10 aa, Epstein-Barr virus
IYLLEMLWRL

SEQ ID NO: 2033 - protein, 10 aa, Epstein-Barr virus
LYLQQNWWTL

SEQ ID NO: 2034 - protein, 9 aa, Epstein-Barr virus
DLILLIIAL

SEQ ID NO: 2035 - protein, 9 aa, Epstein-Barr virus
ATIWQLLAF

SEQ ID NO: 2036 - protein, 9 aa, Epstein-Barr virus
LIIIILIIF

SEQ ID NO: 2037 - protein, 9 aa, Epstein-Barr virus
EVENKGGDQ

SEQ ID NO: 2038 - protein, 10 aa, Epstein-Barr virus
LVDLLWLLLF

SEQ ID NO: 2039 - protein, 11 aa, Epstein-Barr virus
IIIILIIFIFR

SEQ ID NO: 2040 - protein, 9 aa, Epstein-Barr virus
IILIIFIFR

SEQ ID NO: 2041 - protein, 10 aa, Epstein-Barr virus
IIILIIFIFR

SEQ ID NO: 2042 - protein, 9 aa, Epstein-Barr virus
ILIIFIFRR

SEQ ID NO: 2043 - protein, 10 aa, Epstein-Barr virus
DLERGPPGPR

SEQ ID NO: 2044 - protein, 9 aa, Epstein-Barr virus
DPHLPTLLL

SEQ ID NO: 2045 - protein, 9 aa, Epstein-Barr virus
PPLSSSLGL

SEQ ID NO: 2046 - protein, 9 aa, Epstein-Barr virus
GPPLCSQNL

SEQ ID NO: 2047 - protein, 10 aa, Epstein-Barr virus
GPPLSSSLGL

SEQ ID NO: 2048 - protein, 10 aa, Epstein-Barr virus
CPLGALCILL

SEQ ID NO: 2049 - protein, 8 aa, Epstein-Barr virus
FIFRRDLL

SEQ ID NO: 2050 - protein, 9 aa, Epstein-Barr virus
SNEGRHHLL

SEQ ID NO: 2051 - protein, 9 aa, Epstein-Barr virus
SLGLALLLL

SEQ ID NO: 2052 - protein, 9 aa, Epstein-Barr virus
ILLLMITLL

SEQ ID NO: 2053 - protein, 9 aa, Epstein-Barr virus
DLILLIIAL

SEQ ID NO: 2054 - protein, 9 aa, Epstein-Barr virus
ALFLGIVLF

SEQ ID NO: 2055 - protein, 10 aa, Epstein-Barr virus
CLLVLGIWIY

SEQ ID NO: 2056 - protein, 10 aa, Epstein-Barr virus
LLLALLFWLY

SEQ ID NO: 2057 - protein, 10 aa, Epstein-Barr virus
MLIIIILIIF

SEQ ID NO: 2058 - protein, 10 aa, Epstein-Barr virus
DLILLIIALY

SEQ ID NO: 2059 - protein, 9 aa, Epstein-Barr virus
DEHHHDDSL

SEQ ID NO: 2060 - protein, 9 aa, Epstein-Barr virus
DLILLIIAL

SEQ ID NO: 2061 - protein, 8 aa, Epstein-Barr virus
NEGRHHLL

SEQ ID NO: 2062 - protein, 8 aa, Epstein-Barr virus
DLLWLLLF

SEQ ID NO: 2063 - protein, 8 aa, Epstein-Barr virus
EEVENKGG

SEQ ID NO: 2064 - protein, 10 aa, Epstein-Barr virus
WRLGATIWQL

SEQ ID NO: 2065 - protein, 10 aa, Epstein-Barr virus
PRGPPLSSSL

SEQ ID NO: 2066 - protein, 9 aa, Epstein-Barr virus
RRPPRGPPL

SEQ ID NO: 2067 - protein, 9 aa, Epstein-Barr virus
ERGPPGPRR

SEQ ID NO: 2068 - protein, 9 aa, Epstein-Barr virus
FRRDLLCPL

SEQ ID NO: 2069 - protein, 9 aa, Epstein-Barr virus
PPLSSSLGL

SEQ ID NO: 2070 - protein, 9 aa, Epstein-Barr virus
DPHLPTLLL

SEQ ID NO: 2071 - protein, 9 aa, Epstein-Barr virus
GPPLCSQNL

SEQ ID NO: 2072 - protein, 10 aa, Epstein-Barr virus
CPLGALCILL

SEQ ID NO: 2073 - protein, 10 aa, Epstein-Barr virus
DPHGPVQLSY

SEQ ID NO: 2074 - protein, 9 aa, Epstein-Barr virus
SSLGLALLL

SEQ ID NO: 2075 - protein, 9 aa, Epstein-Barr virus
ITLLLIALW

SEQ ID NO: 2076 - protein, 9 aa, Epstein-Barr virus
LSSSLGLAL

SEQ ID NO: 2077 - protein, 10 aa, Epstein-Barr virus
SSSLGLALLL

SEQ ID NO: 2078 - protein, 10 aa, Epstein-Barr virus
NSNEGRHHLL

SEQ ID NO: 2079 - protein, 15 aa, Epstein-Barr virus
LALLLLLLALLFWLY

SEQ ID NO: 2080 - protein, 15 aa, Epstein-Barr virus
RDLLCPLGALCILLL

SEQ ID NO: 2081 - protein, 15 aa, Epstein-Barr virus
LIALWNLHGQALFLG

SEQ ID NO: 2082 - protein, 15 aa, Epstein-Barr virus
GATIWQLLAFFLAFF

SEQ ID NO: 2083 - protein, 15 aa, Epstein-Barr virus
LGIVLFIFGCLLVLG

SEQ ID NO: 2084 - protein, 15 aa, Epstein-Barr virus
WWTLLVDLLWLLLFL

SEQ ID NO: 2085 - protein, 15 aa, Epstein-Barr virus
IFIFRRDLLCPLGAL

SEQ ID NO: 2086 - protein, 15 aa, Epstein-Barr virus
IILIIFIFRRDLLCP

SEQ ID NO: 2087 - protein, 15 aa, Epstein-Barr virus
ILIIFIFRRDLLCPL

SEQ ID NO: 2088 - protein, 15 aa, Epstein-Barr virus
GLALLLLLLALLFWL

SEQ ID NO: 2089 - protein, 15 aa, Epstein-Barr virus
LFWLYIVMSDWTGGA

SEQ ID NO: 2090 - protein, 15 aa, Epstein-Barr virus
LYIVMSDWTGGALLV

SEQ ID NO: 2091 - protein, 15 aa, Epstein-Barr virus
GGALLVLYSFALMLI

SEQ ID NO: 2092 - protein, 15 aa, Epstein-Barr virus
IILIIFIFRRDLLCP

SEQ ID NO: 2093 - protein, 15 aa, Epstein-Barr virus
ILIIFIFRRDLLCPL

SEQ ID NO: 2094 - protein, 15 aa, Epstein-Barr virus
HDPLPHSPSDSAGND

SEQ ID NO: 2095 - protein, 15 aa, Epstein-Barr virus
GGALLVLYSFALMLI

SEQ ID NO: 2096 - protein, 15 aa, Epstein-Barr virus
LVLYSFALMLIIIIL

SEQ ID NO: 2097 - protein, 15 aa, Epstein-Barr virus
LCILLLMITLLLIAL

SEQ ID NO: 2098 - protein, 15 aa, Epstein-Barr virus
LWRLGATIWQLLAFF

SEQ ID NO: 2099 - protein, 15 aa, Epstein-Barr virus
IYLLEMLWRLGATIW

SEQ ID NO: 2100 - protein, 15 aa, Epstein-Barr virus
FWLYIVMSDWTGGAL

SEQ ID NO: 2101 - protein, 15 aa, Epstein-Barr virus
ATIWQLLAFFLAFFL

SEQ ID NO: 2102 - protein, 15 aa, Epstein-Barr virus
IILIIFIFRRDLLCP

SEQ ID NO: 2103 - protein, 15 aa, Epstein-Barr virus
QNWWTLLVDLLWLLL

SEQ ID NO: 2104 - protein, 15 aa, Epstein-Barr virus
WQLLAFFLAFFLDLI

SEQ ID NO: 2105 - protein, 15 aa, Epstein-Barr virus
LLWLLLFLAILIWMY

SEQ ID NO: 2106 - protein, 15 aa, Epstein-Barr virus
LLALLFWLYIVMSDW

SEQ ID NO: 2107 - protein, 15 aa, Epstein-Barr virus
LLVLYSFALMLIIII

SEQ ID NO: 2108 - protein, 15 aa, Epstein-Barr virus
GIVLFIFGCLLVLGI

SEQ ID NO: 2109 - protein, 497 aa, Epstein-Barr virus
MGSLEMVPMGAGPPSPGGDPDGYDGGNNSQYPSASGSSGNTPTPPNDEERESNEEPPPPYEDPYWGNGDRHSDYQPLGTQDQSLYLGLQHDGNDGLPPPPYSPRDDSSQHIYEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 2110 - protein, 9 aa, Epstein-Barr virus
RDDSSQHIY

SEQ ID NO: 2111 - protein, 10 aa, Epstein-Barr virus
ESEERPPTPY

SEQ ID NO: 2112 - protein, 10 aa, Epstein-Barr virus
GYDGGNNSQY

SEQ ID NO: 2113 - protein, 10 aa, Epstein-Barr virus
GNDGLPPPPY

SEQ ID NO: 2114 - protein, 11 aa, Epstein-Barr virus
LTEWGSGNRTY

SEQ ID NO: 2115 - protein, 9 aa, Epstein-Barr virus
LLLAFVLWL

SEQ ID NO: 2116 - protein, 9 aa, Epstein-Barr virus
FLLMLLWTL

SEQ ID NO: 2117 - protein, 10 aa, Epstein-Barr virus
LLASLILGTL

SEQ ID NO: 2118 - protein, 10 aa, Epstein-Barr virus
LLARLFLYAL

SEQ ID NO: 2119 - protein, 10 aa, Epstein-Barr virus
FLIGFALFGV

SEQ ID NO: 2120 - protein, 9 aa, Epstein-Barr virus
LLAAVASSY

SEQ ID NO: 2121 - protein, 9 aa, Epstein-Barr virus
ALIAGGSIL

SEQ ID NO: 2122 - protein, 9 aa, Epstein-Barr virus
SLLLLAAVA

SEQ ID NO: 2123 - protein, 10 aa, Epstein-Barr virus
LLLAAVASSY

SEQ ID NO: 2124 - protein, 10 aa, Epstein-Barr virus
QLSPLLGAVT

SEQ ID NO: 2125 - protein, 9 aa, Epstein-Barr virus
SSYAAAQRK

SEQ ID NO: 2126 - protein, 9 aa, Epstein-Barr virus
GSILQTNFK

SEQ ID NO: 2127 - protein, 10 aa, Epstein-Barr virus
SSCSSCPLSK

SEQ ID NO: 2128 - protein, 10 aa, Epstein-Barr virus
ASSYAAAQRK

SEQ ID NO: 2129 - protein, 10 aa, Epstein-Barr virus
AVLQLSPLLG

SEQ ID NO: 2130 - protein, 9 aa, Epstein-Barr virus
TYGPVFMCL

SEQ ID NO: 2131 - protein, 9 aa, Epstein-Barr virus
PYLFWLAAI

SEQ ID NO: 2132 - protein, 10 aa, Epstein-Barr virus
SYAAAQRKLL

SEQ ID NO: 2133 - protein, 10 aa, Epstein-Barr virus
IYVLVMLVLL

SEQ ID NO: 2134 - protein, 10 aa, Epstein-Barr virus
MFLACVLVLI

SEQ ID NO: 2135 - protein, 9 aa, Epstein-Barr virus
PVFMCLGGL

SEQ ID NO: 2136 - protein, 9 aa, Epstein-Barr virus
DAVLQLSPL

SEQ ID NO: 2137 - protein, 10 aa, Epstein-Barr virus
TVVSMTLLLL

SEQ ID NO: 2138 - protein, 10 aa, Epstein-Barr virus
TVVTATGLAL

SEQ ID NO: 2139 - protein, 10 aa, Epstein-Barr virus
VTVLTAVVTF

SEQ ID NO: 2140 - protein, 11 aa, Epstein-Barr virus
AVASSYAAAQR

SEQ ID NO: 2141 - protein, 11 aa, Epstein-Barr virus
VTFFAICLTWR

SEQ ID NO: 2142 - protein, 9 aa, Epstein-Barr virus
LVLLILAYR

SEQ ID NO: 2143 - protein, 9 aa, Epstein-Barr virus
PLSKILLAR

SEQ ID NO: 2144 - protein, 10 aa, Epstein-Barr virus
VASSYAAAQR

SEQ ID NO: 2145 - protein, 9 aa, Epstein-Barr virus
LPVIVAPYL

SEQ ID NO: 2146 - protein, 9 aa, Epstein-Barr virus
APYLFWLAA

SEQ ID NO: 2147 - protein, 10 aa, Epstein-Barr virus
IPNLFCMLLL

SEQ ID NO: 2148 - protein, 10 aa, Epstein-Barr virus
QPLGTQDQSL

SEQ ID NO: 2149 - protein, 10 aa, Epstein-Barr virus
SPGGLGTLGA

SEQ ID NO: 2150 - protein, 8 aa, Epstein-Barr virus
CPLSKILL

SEQ ID NO: 2151 - protein, 8 aa, Epstein-Barr virus
ILLARLFL

SEQ ID NO: 2152 - protein, 8 aa, Epstein-Barr virus
AAAQRKLL

SEQ ID NO: 2153 - protein, 9 aa, Epstein-Barr virus
AYRRRWRRL

SEQ ID NO: 2154 - protein, 9 aa, Epstein-Barr virus
LARLFLYAL

SEQ ID NO: 2155 - protein, 9 aa, Epstein-Barr virus
MLVLLILAY

SEQ ID NO: 2156 - protein, 9 aa, Epstein-Barr virus
CLPVIVAPY

SEQ ID NO: 2157 - protein, 9 aa, Epstein-Barr virus
LLAAVASSY

SEQ ID NO: 2158 - protein, 10 aa, Epstein-Barr virus
LLLAAVASSY

SEQ ID NO: 2159 - protein, 10 aa, Epstein-Barr virus
RLTVCGGIMF

SEQ ID NO: 2160 - protein, 9 aa, Epstein-Barr virus
SEERPPTPY

SEQ ID NO: 2161 - protein, 9 aa, Epstein-Barr virus
IEDPPFNSL

SEQ ID NO: 2162 - protein, 8 aa, Epstein-Barr virus
EERPPTPY

SEQ ID NO: 2163 - protein, 8 aa, Epstein-Barr virus
TEFIPNLF

SEQ ID NO: 2164 - protein, 8 aa, Epstein-Barr virus
NEEPPPPY

SEQ ID NO: 2165 - protein, 10 aa, Epstein-Barr virus
ARLFLYALAL

SEQ ID NO: 2166 - protein, 10 aa, Epstein-Barr virus
GRGSMNPVCL

SEQ ID NO: 2167 - protein, 10 aa, Epstein-Barr virus
RRLTVCGGIM

SEQ ID NO: 2168 - protein, 9 aa, Epstein-Barr virus
GGLQGIYVL

SEQ ID NO: 2169 - protein, 9 aa, Epstein-Barr virus
CRYCCYYCL

SEQ ID NO: 2170 - protein, 9 aa, Epstein-Barr virus
LPVIVAPYL

SEQ ID NO: 2171 - protein, 9 aa, Epstein-Barr virus
SPGGDPDGY

SEQ ID NO: 2172 - protein, 10 aa, Epstein-Barr virus
QPLGTQDQSL

SEQ ID NO: 2173 - protein, 10 aa, Epstein-Barr virus
PPFNSLLFAL

SEQ ID NO: 2174 - protein, 10 aa, Epstein-Barr virus
GPVFMCLGGL

SEQ ID NO: 2175 - protein, 9 aa, Epstein-Barr virus
KSLSSTEFI

SEQ ID NO: 2176 - protein, 9 aa, Epstein-Barr virus
SSCPLSKIL

SEQ ID NO: 2177 - protein, 10 aa, Epstein-Barr virus
LSKILLARLF

SEQ ID NO: 2178 - protein, 10 aa, Epstein-Barr virus
SSYAAAQRKL

SEQ ID NO: 2179 - protein, 10 aa, Epstein-Barr virus
LSSPGGLGTL

SEQ ID NO: 2180 - protein, 15 aa, Epstein-Barr virus
QTNFKSLSSTEFIPN

SEQ ID NO: 2181 - protein, 15 aa, Epstein-Barr virus
ALSLLLLAAVASSYA

SEQ ID NO: 2182 - protein, 15 aa, Epstein-Barr virus
PGGLGTLGAALLTLA

SEQ ID NO: 2183 - protein, 15 aa, Epstein-Barr virus
CMLLLIVAGILFILA

SEQ ID NO: 2184 - protein, 15 aa, Epstein-Barr virus
AGFLIFLIGFALFGV

SEQ ID NO: 2185 - protein, 15 aa, Epstein-Barr virus
YLGLQHDGNDGLPPP

SEQ ID NO: 2186 - protein, 15 aa, Epstein-Barr virus
VLVLIVDAVLQLSPL

SEQ ID NO: 2187 - protein, 15 aa, Epstein-Barr virus
AVWLTVMSNTLLSAW

SEQ ID NO: 2188 - protein, 15 aa, Epstein-Barr virus
YQPLGTQDQSLYLGL

SEQ ID NO: 2189 - protein, 15 aa, Epstein-Barr virus
VLVMLVLLILAYRRR

SEQ ID NO: 2190 - protein, 15 aa, Epstein-Barr virus
HSDYQPLGTQDQSLY

SEQ ID NO: 2191 - protein, 15 aa, Epstein-Barr virus
QSLYLGLQHDGNDGL

SEQ ID NO: 2192 - protein, 15 aa, Epstein-Barr virus
QHIYEEAGRGSMNPV

SEQ ID NO: 2193 - protein, 15 aa, Epstein-Barr virus
ASSYAAAQRKLLTPV

SEQ ID NO: 2194 - protein, 15 aa, Epstein-Barr virus
GAVWLTVMSNTLLSA

SEQ ID NO: 2195 - protein, 15 aa, Epstein-Barr virus
ASCFTASVSTVVTAT

SEQ ID NO: 2196 - protein, 15 aa, Epstein-Barr virus
ACVLVLIVDAVLQLS

SEQ ID NO: 2197 - protein, 15 aa, Epstein-Barr virus
VTFFAICLTWRIEDP

SEQ ID NO: 2198 - protein, 15 aa, Epstein-Barr virus
GAVWLTVMSNTLLSA

SEQ ID NO: 2199 - protein, 15 aa, Epstein-Barr virus
LSAWILTAGFLIFLI

SEQ ID NO: 2200 - protein, 15 aa, Epstein-Barr virus
QSLYLGLQHDGNDGL

SEQ ID NO: 2201 - protein, 15 aa, Epstein-Barr virus
LTEWGSGNRTYGPVF

SEQ ID NO: 2202 - protein, 15 aa, Epstein-Barr virus
PNLFCMLLLIVAGIL

SEQ ID NO: 2203 - protein, 15 aa, Epstein-Barr virus
NRTYGPVFMCLGGLL

SEQ ID NO: 2204 - protein, 15 aa, Epstein-Barr virus
VTVLTAVVTFFAICL

SEQ ID NO: 2205 - protein, 15 aa, Epstein-Barr virus
LTAVVTFFAICLTWR

SEQ ID NO: 2206 - protein, 15 aa, Epstein-Barr virus
FLLMLLWTLVVLLIC

SEQ ID NO: 2207 - protein, 15 aa, Epstein-Barr virus
LIFLIGFALFGVIRC

SEQ ID NO: 2208 - protein, 15 aa, Epstein-Barr virus
LAILTEWGSGNRTYG

SEQ ID NO: 2209 - protein, 15 aa, Epstein-Barr virus
LIGFALFGVIRCCRY

SEQ ID NO: 2210 - protein, 641 aa, Epstein-Barr virus
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGAGAGAGGAGAGGAGAGGGAGAGGGAGGAGGAGGAGAGGGAGAGGGAGGAGGAGAGGGAGAGGGAGGAGAGGGAGGAGGAGAGGGAGAGGGAGGAGAGGGAGGAGGAGAGGGAGAGGAGGAGGAGAGGAGAGGGAGGAGGAGAGGAGAGGAGAGGAGAGGAGGAGAGGAGGAGAGGAGGAGAGGGAGGAGAGGGAGGAGAGGAGGAGAGGAGGAGAGGAGGAGAGGGAGAGGAGAGGGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 2211 - protein, 9 aa, Epstein-Barr virus
VGEADYFEY

SEQ ID NO: 2212 - protein, 9 aa, Epstein-Barr virus
TTDEGTWVA

SEQ ID NO: 2213 - protein, 9 aa, Epstein-Barr virus
TWVAGVFVY

SEQ ID NO: 2214 - protein, 10 aa, Epstein-Barr virus
GTWVAGVFVY

SEQ ID NO: 2215 - protein, 11 aa, Epstein-Barr virus
FVYGGSKTSLY

SEQ ID NO: 2216 - protein, 9 aa, Epstein-Barr virus
NIAEGLRAL

SEQ ID NO: 2217 - protein, 9 aa, Epstein-Barr virus
ALAIPQCRL

SEQ ID NO: 2218 - protein, 9 aa, Epstein-Barr virus
VLKDAIKDL

SEQ ID NO: 2219 - protein, 10 aa, Epstein-Barr virus
FLQTHIFAEV

SEQ ID NO: 2220 - protein, 10 aa, Epstein-Barr virus
AIPQCRLTPL

SEQ ID NO: 2221 - protein, 9 aa, Epstein-Barr virus
AIKDLVMTK

SEQ ID NO: 2222 - protein, 9 aa, Epstein-Barr virus
GVFVYGGSK

SEQ ID NO: 2223 - protein, 10 aa, Epstein-Barr virus
ALLARSHVER

SEQ ID NO: 2224 - protein, 10 aa, Epstein-Barr virus
RLTPLSRLPF

SEQ ID NO: 2225 - protein, 10 aa, Epstein-Barr virus
GLRALLARSH

SEQ ID NO: 2226 - protein, 9 aa, Epstein-Barr virus
GVFVYGGSK

SEQ ID NO: 2227 - protein, 9 aa, Epstein-Barr virus
GSGSGPRHR

SEQ ID NO: 2228 - protein, 10 aa, Epstein-Barr virus
QTHIFAEVLK

SEQ ID NO: 2229 - protein, 10 aa, Epstein-Barr virus
ALLARSHVER

SEQ ID NO: 2230 - protein, 10 aa, Epstein-Barr virus
GSKTSLYNLR

SEQ ID NO: 2231 - protein, 9 aa, Epstein-Barr virus
VYGGSKTSL

SEQ ID NO: 2232 - protein, 10 aa, Epstein-Barr virus
LYNLRRGTAL

SEQ ID NO: 2233 - protein, 9 aa, Epstein-Barr virus
KFENIAEGL

SEQ ID NO: 2234 - protein, 10 aa, Epstein-Barr virus
IFAEVLKDAI

SEQ ID NO: 2235 - protein, 10 aa, Epstein-Barr virus
YFMVFLQTHI

SEQ ID NO: 2236 - protein, 9 aa, Epstein-Barr virus
MVFLQTHIF

SEQ ID NO: 2237 - protein, 9 aa, Epstein-Barr virus
DGVDLPPWF

SEQ ID NO: 2238 - protein, 10 aa, Epstein-Barr virus
EVLKDAIKDL

SEQ ID NO: 2239 - protein, 10 aa, Epstein-Barr virus
ENIAEGLRAL

SEQ ID NO: 2240 - protein, 10 aa, Epstein-Barr virus
DVPPGAIEQG

SEQ ID NO: 2241 - protein, 11 aa, Epstein-Barr virus
RALLARSHVER

SEQ ID NO: 2242 - protein, 10 aa, Epstein-Barr virus
GTALAIPQCR

SEQ ID NO: 2243 - protein, 10 aa, Epstein-Barr virus
QTHIFAEVLK

SEQ ID NO: 2244 - protein, 10 aa, Epstein-Barr virus
DAIKDLVMTK

SEQ ID NO: 2245 - protein, 11 aa, Epstein-Barr virus
MTKPAPTCNIR

SEQ ID NO: 2246 - protein, 9 aa, Epstein-Barr virus
IPQCRLTPL

SEQ ID NO: 2247 - protein, 9 aa, Epstein-Barr virus
GPGPQPGPL

SEQ ID NO: 2248 - protein, 9 aa, Epstein-Barr virus
EPDVPPGAI

SEQ ID NO: 2249 - protein, 10 aa, Epstein-Barr virus
GPGTGPGNGL

SEQ ID NO: 2250 - protein, 10 aa, Epstein-Barr virus
RPPPGRRPFF

SEQ ID NO: 2251 - protein, 8 aa, Epstein-Barr virus
NLRRGTAL

SEQ ID NO: 2252 - protein, 8 aa, Epstein-Barr virus
GPRHRDGV

SEQ ID NO: 2253 - protein, 9 aa, Epstein-Barr virus
VLKDAIKDL

SEQ ID NO: 2254 - protein, 9 aa, Epstein-Barr virus
IPQCRLTPL

SEQ ID NO: 2255 - protein, 9 aa, Epstein-Barr virus
GRRKKGGWF

SEQ ID NO: 2256 - protein, 9 aa, Epstein-Barr virus
PLRESIVCY

SEQ ID NO: 2257 - protein, 10 aa, Epstein-Barr virus
RLTPLSRLPF

SEQ ID NO: 2258 - protein, 9 aa, Epstein-Barr virus
GQGGSNPKF

SEQ ID NO: 2259 - protein, 10 aa, Epstein-Barr virus
PVGEADYFEY

SEQ ID NO: 2260 - protein, 9 aa, Epstein-Barr virus
MVFLQTHIF

SEQ ID NO: 2261 - protein, 8 aa, Epstein-Barr virus
RESIVCYF

SEQ ID NO: 2262 - protein, 8 aa, Epstein-Barr virus
GEADYFEY

SEQ ID NO: 2263 - protein, 8 aa, Epstein-Barr virus
FENIAEGL

SEQ ID NO: 2264 - protein, 8 aa, Epstein-Barr virus
AEGLRALL

SEQ ID NO: 2265 - protein, 9 aa, Epstein-Barr virus
DGVDLPPWF

SEQ ID NO: 2266 - protein, 10 aa, Epstein-Barr virus
RRKKGGWFGK

SEQ ID NO: 2267 - protein, 10 aa, Epstein-Barr virus
GRGGSGGRGR

SEQ ID NO: 2268 - protein, 9 aa, Epstein-Barr virus
GRGGSGGRR

SEQ ID NO: 2269 - protein, 9 aa, Epstein-Barr virus
RRGGDNHGR

SEQ ID NO: 2270 - protein, 9 aa, Epstein-Barr virus
CRLTPLSRL

SEQ ID NO: 2271 - protein, 9 aa, Epstein-Barr virus
HPVGEADYF

SEQ ID NO: 2272 - protein, 9 aa, Epstein-Barr virus
IPQCRLTPL

SEQ ID NO: 2273 - protein, 9 aa, Epstein-Barr virus
GPGPQPGPL

SEQ ID NO: 2274 - protein, 10 aa, Epstein-Barr virus
GPLRESIVCY

SEQ ID NO: 2275 - protein, 10 aa, Epstein-Barr virus
GPGTGPGNGL

SEQ ID NO: 2276 - protein, 9 aa, Epstein-Barr virus
GSNPKFENI

SEQ ID NO: 2277 - protein, 9 aa, Epstein-Barr virus
GSKTSLYNL

SEQ ID NO: 2278 - protein, 9 aa, Epstein-Barr virus
IAEGLRALL

SEQ ID NO: 2279 - protein, 10 aa, Epstein-Barr virus
ESIVCYFMVF

SEQ ID NO: 2280 - protein, 10 aa, Epstein-Barr virus
MTKPAPTCNI

SEQ ID NO: 2281 - protein, 15 aa, Epstein-Barr virus
CYFMVFLQTHIFAEV

SEQ ID NO: 2282 - protein, 15 aa, Epstein-Barr virus
TSLYNLRRGTALAIP

SEQ ID NO: 2283 - protein, 15 aa, Epstein-Barr virus
RLPFGMAPGPGPQPG

SEQ ID NO: 2284 - protein, 15 aa, Epstein-Barr virus
AEGLRALLARSHVER

SEQ ID NO: 2285 - protein, 15 aa, Epstein-Barr virus
AGVFVYGGSKTSLYN

SEQ ID NO: 2286 - protein, 15 aa, Epstein-Barr virus
FAEVLKDAIKDLVMT

SEQ ID NO: 2287 - protein, 15 aa, Epstein-Barr virus
FENIAEGLRALLARS

SEQ ID NO: 2288 - protein, 15 aa, Epstein-Barr virus
QCRLTPLSRLPFGMA

SEQ ID NO: 2289 - protein, 15 aa, Epstein-Barr virus
RPFFHPVGEADYFEY

SEQ ID NO: 2290 - protein, 15 aa, Epstein-Barr virus
GVFVYGGSKTSLYNL

SEQ ID NO: 2291 - protein, 15 aa, Epstein-Barr virus
VCYFMVFLQTHIFAE

SEQ ID NO: 2292 - protein, 15 aa, Epstein-Barr virus
KTSLYNLRRGTALAI

SEQ ID NO: 2293 - protein, 15 aa, Epstein-Barr virus
AGVFVYGGSKTSLYN

SEQ ID NO: 2294 - protein, 15 aa, Epstein-Barr virus
THIFAEVLKDAIKDL

SEQ ID NO: 2295 - protein, 15 aa, Epstein-Barr virus
FENIAEGLRALLARS

SEQ ID NO: 2296 - protein, 15 aa, Epstein-Barr virus
VFVYGGSKTSLYNLR

SEQ ID NO: 2297 - protein, 15 aa, Epstein-Barr virus
RPFFHPVGEADYFEY

SEQ ID NO: 2298 - protein, 15 aa, Epstein-Barr virus
NPKFENIAEGLRALL

SEQ ID NO: 2299 - protein, 15 aa, Epstein-Barr virus
RSHVERTTDEGTWVA

SEQ ID NO: 2300 - protein, 15 aa, Epstein-Barr virus
CYFMVFLQTHIFAEV

SEQ ID NO: 2301 - protein, 15 aa, Epstein-Barr virus
KTSLYNLRRGTALAI

SEQ ID NO: 2302 - protein, 15 aa, Epstein-Barr virus
KGGWFGKHRGQGGSN

SEQ ID NO: 2303 - protein, 15 aa, Epstein-Barr virus
THIFAEVLKDAIKDL

SEQ ID NO: 2304 - protein, 15 aa, Epstein-Barr virus
PPWFPPMVEGAAAEG

SEQ ID NO: 2305 - protein, 15 aa, Epstein-Barr virus
QCRLTPLSRLPFGMA

SEQ ID NO: 2306 - protein, 15 aa, Epstein-Barr virus
RESIVCYFMVFLQTH

SEQ ID NO: 2307 - protein, 15 aa, Epstein-Barr virus
ESIVCYFMVFLQTHI

SEQ ID NO: 2308 - protein, 15 aa, Epstein-Barr virus
VAGVFVYGGSKTSLY

SEQ ID NO: 2309 - protein, 15 aa, Epstein-Barr virus
GVDLPPWFPPMVEGA

SEQ ID NO: 2310 - protein, 15 aa, Epstein-Barr virus
LYNLRRGTALAIPQC

SEQ ID NO: 2311 - protein, 487 aa, Epstein-Barr virus
MPTFYLALHGGQTYHLIVDTDSLGNPSLSVIPSNPYQEQLSDTPLIPLTIFVGENTGVPPPLPPPPPPPPPPPPPPPPPPPPPPPPPPSPPPPPPPPPPPQRRDAWTQEPSPLDRDPLGYDVGHGPLASAMRMLWMANYIVRQSRGDRGLILPQGPQTAPQARLVQPHVPPLRPTAPTILSPLSQPRLTPPQPLMMPPRPTPPTPLPPATLTVPPRPTRPTTLPPTPLLTVLQRPTELQPTPSPPRMHLPVLHVPDQSMHPLTHQSTPNDPDSPEPRSPTVFYNIPPMPLPPSQLPPPAAPAQPPPGVINDQQLHHLPSGPPWWPPICDPPQPSKTQGQSRGQSRGRGRGRGRGRGKGKSRDKQRKPGGPWRPEPNTSSPSMPELSPVLGLHQGQGAGDSPTPGPSNAAPVCRNSHTATPNVSPIHEPESHNSPEAPILFPDDWYPPSIDPADLDESWDYIFETTESPSSDEDYVEGPSKRPRPSIQ

SEQ ID NO: 2312 - protein, 9 aa, Epstein-Barr virus
PLDRDPLGY

SEQ ID NO: 2313 - protein, 10 aa, Epstein-Barr virus
PADLDESWDY

SEQ ID NO: 2314 - protein, 11 aa, Epstein-Barr virus
TTESPSSDEDY

SEQ ID NO: 2315 - protein, 11 aa, Epstein-Barr virus
SPEPRSPTVFY

SEQ ID NO: 2316 - protein, 11 aa, Epstein-Barr virus
PSPLDRDPLGY

SEQ ID NO: 2317 - protein, 9 aa, Epstein-Barr virus
HLIVDTDSL

SEQ ID NO: 2318 - protein, 9 aa, Epstein-Barr virus
SLGNPSLSV

SEQ ID NO: 2319 - protein, 10 aa, Epstein-Barr virus
TLPPTPLLTV

SEQ ID NO: 2320 - protein, 10 aa, Epstein-Barr virus
VINDQQLHHL

SEQ ID NO: 2321 - protein, 10 aa, Epstein-Barr virus
ALHGGQTYHL

SEQ ID NO: 2322 - protein, 9 aa, Epstein-Barr virus
RGRGRGRGK

SEQ ID NO: 2323 - protein, 9 aa, Epstein-Barr virus
PLDRDPLGY

SEQ ID NO: 2324 - protein, 10 aa, Epstein-Barr virus
YLALHGGQTY

SEQ ID NO: 2325 - protein, 10 aa, Epstein-Barr virus
RLTPPQPLMM

SEQ ID NO: 2326 - protein, 10 aa, Epstein-Barr virus
SVIPSNPYQE

SEQ ID NO: 2327 - protein, 9 aa, Epstein-Barr virus
PSNAAPVCR

SEQ ID NO: 2328 - protein, 10 aa, Epstein-Barr virus
WTQEPSPLDR

SEQ ID NO: 2329 - protein, 10 aa, Epstein-Barr virus
PTPLLTVLQR

SEQ ID NO: 2330 - protein, 10 aa, Epstein-Barr virus
LVQPHVPPLR

SEQ ID NO: 2331 - protein, 10 aa, Epstein-Barr virus
MLWMANYIVR

SEQ ID NO: 2332 - protein, 9 aa, Epstein-Barr virus
GYDVGHGPL

SEQ ID NO: 2333 - protein, 9 aa, Epstein-Barr virus
FYNIPPMPL

SEQ ID NO: 2334 - protein, 9 aa, Epstein-Barr virus
NSPEAPILF

SEQ ID NO: 2335 - protein, 9 aa, Epstein-Barr virus
TTLPPTPLL

SEQ ID NO: 2336 - protein, 9 aa, Epstein-Barr virus
TVLQRPTEL

SEQ ID NO: 2337 - protein, 9 aa, Epstein-Barr virus
LVQPHVPPL

SEQ ID NO: 2338 - protein, 9 aa, Epstein-Barr virus
ETTESPSSD

SEQ ID NO: 2339 - protein, 10 aa, Epstein-Barr virus
DTPLIPLTIF

SEQ ID NO: 2340 - protein, 10 aa, Epstein-Barr virus
DTDSLGNPSL

SEQ ID NO: 2341 - protein, 10 aa, Epstein-Barr virus
DVGHGPLASA

SEQ ID NO: 2342 - protein, 11 aa, Epstein-Barr virus
PTILSPLSQPR

SEQ ID NO: 2343 - protein, 9 aa, Epstein-Barr virus
PATLTVPPR

SEQ ID NO: 2344 - protein, 10 aa, Epstein-Barr virus
PTPLLTVLQR

SEQ ID NO: 2345 - protein, 10 aa, Epstein-Barr virus
MLWMANYIVR

SEQ ID NO: 2346 - protein, 10 aa, Epstein-Barr virus
LVQPHVPPLR

SEQ ID NO: 2347 - protein, 9 aa, Epstein-Barr virus
TPSPPRMHL

SEQ ID NO: 2348 - protein, 9 aa, Epstein-Barr virus
PPTPLLTVL

SEQ ID NO: 2349 - protein, 10 aa, Epstein-Barr virus
EPSPLDRDPL

SEQ ID NO: 2350 - protein, 10 aa, Epstein-Barr virus
PPRPTPPTPL

SEQ ID NO: 2351 - protein, 10 aa, Epstein-Barr virus
PPRPTRPTTL

SEQ ID NO: 2352 - protein, 8 aa, Epstein-Barr virus
SPLDRDPL

SEQ ID NO: 2353 - protein, 8 aa, Epstein-Barr virus
SKRPRPSI

SEQ ID NO: 2354 - protein, 9 aa, Epstein-Barr virus
PPRMHLPVL

SEQ ID NO: 2355 - protein, 9 aa, Epstein-Barr virus
SRGDRGLIL

SEQ ID NO: 2356 - protein, 9 aa, Epstein-Barr virus
RGKGKSRDK

SEQ ID NO: 2357 - protein, 9 aa, Epstein-Barr virus
PLDRDPLGY

SEQ ID NO: 2358 - protein, 10 aa, Epstein-Barr virus
YLALHGGQTY

SEQ ID NO: 2359 - protein, 10 aa, Epstein-Barr virus
SLSVIPSNPY

SEQ ID NO: 2360 - protein, 10 aa, Epstein-Barr virus
DLDESWDYIF

SEQ ID NO: 2361 - protein, 10 aa, Epstein-Barr virus
PLPPATLTVP

SEQ ID NO: 2362 - protein, 9 aa, Epstein-Barr virus
PEPRSPTVF

SEQ ID NO: 2363 - protein, 8 aa, Epstein-Barr virus
DESWDYIF

SEQ ID NO: 2364 - protein, 9 aa, Epstein-Barr virus
QEQLSDTPL

SEQ ID NO: 2365 - protein, 9 aa, Epstein-Barr virus
PELSPVLGL

SEQ ID NO: 2366 - protein, 8 aa, Epstein-Barr virus
DEDYVEGP

SEQ ID NO: 2367 - protein, 10 aa, Epstein-Barr virus
GRGKGKSRDK

SEQ ID NO: 2368 - protein, 10 aa, Epstein-Barr virus
PRLTPPQPLM

SEQ ID NO: 2369 - protein, 9 aa, Epstein-Barr virus
QRKPGGPWR

SEQ ID NO: 2370 - protein, 9 aa, Epstein-Barr virus
PRPTPPTPL

SEQ ID NO: 2371 - protein, 9 aa, Epstein-Barr virus
PRPTRPTTL

SEQ ID NO: 2372 - protein, 9 aa, Epstein-Barr virus
EPRSPTVFY

SEQ ID NO: 2373 - protein, 9 aa, Epstein-Barr virus
VPDQSMHPL

SEQ ID NO: 2374 - protein, 10 aa, Epstein-Barr virus
LPPTPLLTVL

SEQ ID NO: 2375 - protein, 10 aa, Epstein-Barr virus
SPPRMHLPVL

SEQ ID NO: 2376 - protein, 10 aa, Epstein-Barr virus
SPLDRDPLGY

SEQ ID NO: 2377 - protein, 9 aa, Epstein-Barr virus
RSPTVFYNI

SEQ ID NO: 2378 - protein, 9 aa, Epstein-Barr virus
NSPEAPILF

SEQ ID NO: 2379 - protein, 9 aa, Epstein-Barr virus
LALHGGQTY

SEQ ID NO: 2380 - protein, 10 aa, Epstein-Barr virus
PSGPPWWPPI

SEQ ID NO: 2381 - protein, 10 aa, Epstein-Barr virus
PSMPELSPVL

SEQ ID NO: 2382 - protein, 15 aa, Epstein-Barr virus
QPHVPPLRPTAPTIL

SEQ ID NO: 2383 - protein, 15 aa, Epstein-Barr virus
SNPYQEQLSDTPLIP

SEQ ID NO: 2384 - protein, 15 aa, Epstein-Barr virus
PTVFYNIPPMPLPPS

SEQ ID NO: 2385 - protein, 15 aa, Epstein-Barr virus
DQQLHHLPSGPPWWP

SEQ ID NO: 2386 - protein, 15 aa, Epstein-Barr virus
RGLILPQGPQTAPQA

SEQ ID NO: 2387 - protein, 15 aa, Epstein-Barr virus
PPGVINDQQLHHLPS

SEQ ID NO: 2388 - protein, 15 aa, Epstein-Barr virus
PTILSPLSQPRLTPP

SEQ ID NO: 2389 - protein, 15 aa, Epstein-Barr virus
TYHLIVDTDSLGNPS

SEQ ID NO: 2390 - protein, 15 aa, Epstein-Barr virus
HLIVDTDSLGNPSLS

SEQ ID NO: 2391 - protein, 15 aa, Epstein-Barr virus
ASAMRMLWMANYIVR

SEQ ID NO: 2392 - protein, 15 aa, Epstein-Barr virus
MPTFYLALHGGQTYH

SEQ ID NO: 2393 - protein, 15 aa, Epstein-Barr virus
GGPWRPEPNTSSPSM

SEQ ID NO: 2394 - protein, 15 aa, Epstein-Barr virus
PTILSPLSQPRLTPP

SEQ ID NO: 2395 - protein, 15 aa, Epstein-Barr virus
PTPLLTVLQRPTELQ

SEQ ID NO: 2396 - protein, 15 aa, Epstein-Barr virus
DQSMHPLTHQSTPND

SEQ ID NO: 2397 - protein, 15 aa, Epstein-Barr virus
NPSLSVIPSNPYQEQ

SEQ ID NO: 2398 - protein, 15 aa, Epstein-Barr virus
PADLDESWDYIFETT

SEQ ID NO: 2399 - protein, 15 aa, Epstein-Barr virus
DYIFETTESPSSDED

SEQ ID NO: 2400 - protein, 15 aa, Epstein-Barr virus
YLALHGGQTYHLIVD

SEQ ID NO: 2401 - protein, 15 aa, Epstein-Barr virus
YHLIVDTDSLGNPSL

SEQ ID NO: 2402 - protein, 15 aa, Epstein-Barr virus
GQTYHLIVDTDSLGN

SEQ ID NO: 2403 - protein, 15 aa, Epstein-Barr virus
TPLLTVLQRPTELQP

SEQ ID NO: 2404 - protein, 15 aa, Epstein-Barr virus
PQPLMMPPRPTPPTP

SEQ ID NO: 2405 - protein, 15 aa, Epstein-Barr virus
DQSMHPLTHQSTPND

SEQ ID NO: 2406 - protein, 15 aa, Epstein-Barr virus
VINDQQLHHLPSGPP

SEQ ID NO: 2407 - protein, 15 aa, Epstein-Barr virus
LIPLTIFVGENTGVP

SEQ ID NO: 2408 - protein, 15 aa, Epstein-Barr virus
YHLIVDTDSLGNPSL

SEQ ID NO: 2409 - protein, 15 aa, Epstein-Barr virus
TIFVGENTGVPPPLP

SEQ ID NO: 2410 - protein, 15 aa, Epstein-Barr virus
MLWMANYIVRQSRGD

SEQ ID NO: 2411 - protein, 15 aa, Epstein-Barr virus
QPRLTPPQPLMMPPR

SEQ ID NO: 2412 - protein, 158 aa, Human papillomavirus
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 2413 - protein, 11 aa, Human papillomavirus
YAVCDKCLKFY

SEQ ID NO: 2414 - protein, 11 aa, Human papillomavirus
SEYRHYCYSLY

SEQ ID NO: 2415 - protein, 11 aa, Human papillomavirus
CKQQLLRREVY

SEQ ID NO: 2416 - protein, 10 aa, Human papillomavirus
IHDIILECVY

SEQ ID NO: 2417 - protein, 10 aa, Human papillomavirus
YSKISEYRHY

SEQ ID NO: 2418 - protein, 10 aa, Human papillomavirus
TIHDIILECV

SEQ ID NO: 2419 - protein, 10 aa, Human papillomavirus
QLCTELQTTI

SEQ ID NO: 2420 - protein, 10 aa, Human papillomavirus
PLCDLLIRCI

SEQ ID NO: 2421 - protein, 9 aa, Human papillomavirus
KLPQLCTEL

SEQ ID NO: 2422 - protein, 9 aa, Human papillomavirus
QLCTELQTT

SEQ ID NO: 2423 - protein, 10 aa, Human papillomavirus
LLIRCINCQK

SEQ ID NO: 2424 - protein, 10 aa, Human papillomavirus
DIILECVYCK

SEQ ID NO: 2425 - protein, 10 aa, Human papillomavirus
CVYCKQQLLR

SEQ ID NO: 2426 - protein, 10 aa, Human papillomavirus
SLYGTTLEQQ

SEQ ID NO: 2427 - protein, 9 aa, Human papillomavirus
IVYRDGNPY

SEQ ID NO: 2428 - protein, 10 aa, Human papillomavirus
CVYCKQQLLR

SEQ ID NO: 2429 - protein, 10 aa, Human papillomavirus
GTTLEQQYNK

SEQ ID NO: 2430 - protein, 10 aa, Human papillomavirus
DIILECVYCK

SEQ ID NO: 2431 - protein, 10 aa, Human papillomavirus
AFRDLCIVYR

SEQ ID NO: 2432 - protein, 10 aa, Human papillomavirus
WTGRCMSCCR

SEQ ID NO: 2433 - protein, 10 aa, Human papillomavirus
QYNKPLCDLL

SEQ ID NO: 2434 - protein, 10 aa, Human papillomavirus
QDPQERPRKL

SEQ ID NO: 2435 - protein, 10 aa, Human papillomavirus
LCPEEKQRHL

SEQ ID NO: 2436 - protein, 9 aa, Human papillomavirus
VYDFAFRDL

SEQ ID NO: 2437 - protein, 9 aa, Human papillomavirus
PYAVCDKCL

SEQ ID NO: 2438 - protein, 10 aa, Human papillomavirus
EVYDFAFRDL

SEQ ID NO: 2439 - protein, 10 aa, Human papillomavirus
AVCDKCLKFY

SEQ ID NO: 2440 - protein, 10 aa, Human papillomavirus
EYRHYCYSLY

SEQ ID NO: 2441 - protein, 10 aa, Human papillomavirus
DIILECVYCK

SEQ ID NO: 2442 - protein, 10 aa, Human papillomavirus
CIVYRDGNPY

SEQ ID NO: 2443 - protein, 11 aa, Human papillomavirus
FAFRDLCIVYR

SEQ ID NO: 2444 - protein, 11 aa, Human papillomavirus
RTAMFQDPQER

SEQ ID NO: 2445 - protein, 11 aa, Human papillomavirus
CVYCKQQLLRR

SEQ ID NO: 2446 - protein, 11 aa, Human papillomavirus
MSCCRSSRTRR

SEQ ID NO: 2447 - protein, 11 aa, Human papillomavirus
DLLIRCINCQK

SEQ ID NO: 2448 - protein, 10 aa, Human papillomavirus
RPRKLPQLCT

SEQ ID NO: 2449 - protein, 10 aa, Human papillomavirus
NPYAVCDKCL

SEQ ID NO: 2450 - protein, 10 aa, Human papillomavirus
LPQLCTELQT

SEQ ID NO: 2451 - protein, 10 aa, Human papillomavirus
QERPRKLPQL

SEQ ID NO: 2452 - protein, 9 aa, Human papillomavirus
DPQERPRKL

SEQ ID NO: 2453 - protein, 9 aa, Human papillomavirus
CPEEKQRHL

SEQ ID NO: 2454 - protein, 9 aa, Human papillomavirus
DPQERPRKL

SEQ ID NO: 2455 - protein, 9 aa, Human papillomavirus
DKKQRFHNI

SEQ ID NO: 2456 - protein, 9 aa, Human papillomavirus
ERPRKLPQL

SEQ ID NO: 2457 - protein, 9 aa, Human papillomavirus
CVYCKQQLL

SEQ ID NO: 2458 - protein, 10 aa, Human papillomavirus
KQQLLRREVY

SEQ ID NO: 2459 - protein, 10 aa, Human papillomavirus
AVCDKCLKFY

SEQ ID NO: 2460 - protein, 10 aa, Human papillomavirus
QLLRREVYDF

SEQ ID NO: 2461 - protein, 10 aa, Human papillomavirus
QLCTELQTTI

SEQ ID NO: 2462 - protein, 10 aa, Human papillomavirus
FAFRDLCIVY

SEQ ID NO: 2463 - protein, 9 aa, Human papillomavirus
LEQQYNKPL

SEQ ID NO: 2464 - protein, 9 aa, Human papillomavirus
QQLLRREVY

SEQ ID NO: 2465 - protein, 9 aa, Human papillomavirus
DPQERPRKL

SEQ ID NO: 2466 - protein, 9 aa, Human papillomavirus
AFRDLCIVY

SEQ ID NO: 2467 - protein, 8 aa, Human papillomavirus
REVYDFAF

SEQ ID NO: 2468 - protein, 10 aa, Human papillomavirus
RREVYDFAFR

SEQ ID NO: 2469 - protein, 10 aa, Human papillomavirus
QRHLDKKQRF

SEQ ID NO: 2470 - protein, 10 aa, Human papillomavirus
IRCINCQKPL

SEQ ID NO: 2471 - protein, 10 aa, Human papillomavirus
LRREVYDFAF

SEQ ID NO: 2472 - protein, 10 aa, Human papillomavirus
RKLPQLCTEL

SEQ ID NO: 2473 - protein, 10 aa, Human papillomavirus
NPYAVCDKCL

SEQ ID NO: 2474 - protein, 10 aa, Human papillomavirus
FAFRDLCIVY

SEQ ID NO: 2475 - protein, 10 aa, Human papillomavirus
LQTTIHDIIL

SEQ ID NO: 2476 - protein, 10 aa, Human papillomavirus
KPLCDLLIRC

SEQ ID NO: 2477 - protein, 10 aa, Human papillomavirus
ECVYCKQQLL

SEQ ID NO: 2478 - protein, 10 aa, Human papillomavirus
YSKISEYRHY

SEQ ID NO: 2479 - protein, 10 aa, Human papillomavirus
EVYDFAFRDL

SEQ ID NO: 2480 - protein, 10 aa, Human papillomavirus
SSRTRRETQL

SEQ ID NO: 2481 - protein, 10 aa, Human papillomavirus
FAFRDLCIVY

SEQ ID NO: 2482 - protein, 10 aa, Human papillomavirus
YAVCDKCLKF

SEQ ID NO: 2483 - protein, 15 aa, Human papillomavirus
IRCINCQKPLCPEEK

SEQ ID NO: 2484 - protein, 15 aa, Human papillomavirus
CIVYRDGNPYAVCDK

SEQ ID NO: 2485 - protein, 15 aa, Human papillomavirus
RHYCYSLYGTTLEQQ

SEQ ID NO: 2486 - protein, 15 aa, Human papillomavirus
NKPLCDLLIRCINCQ

SEQ ID NO: 2487 - protein, 15 aa, Human papillomavirus
DLLIRCINCQKPLCP

SEQ ID NO: 2488 - protein, 15 aa, Human papillomavirus
RTAMFQDPQERPRKL

SEQ ID NO: 2489 - protein, 15 aa, Human papillomavirus
LCIVYRDGNPYAVCD

SEQ ID NO: 2490 - protein, 15 aa, Human papillomavirus
EKQRHLDKKQRFHNI

SEQ ID NO: 2491 - protein, 15 aa, Human papillomavirus
GTTLEQQYNKPLCDL

SEQ ID NO: 2492 - protein, 15 aa, Human papillomavirus
NPYAVCDKCLKFYSK

SEQ ID NO: 2493 - protein, 15 aa, Human papillomavirus
PRKLPQLCTELQTTI

SEQ ID NO: 2494 - protein, 15 aa, Human papillomavirus
LPQLCTELQTTIHDI

SEQ ID NO: 2495 - protein, 15 aa, Human papillomavirus
FRDLCIVYRDGNPYA

SEQ ID NO: 2496 - protein, 15 aa, Human papillomavirus
LCIVYRDGNPYAVCD

SEQ ID NO: 2497 - protein, 15 aa, Human papillomavirus
REVYDFAFRDLCIVY

SEQ ID NO: 2498 - protein, 15 aa, Human papillomavirus
YSLYGTTLEQQYNKP

SEQ ID NO: 2499 - protein, 15 aa, Human papillomavirus
CTELQTTIHDIILEC

SEQ ID NO: 2500 - protein, 15 aa, Human papillomavirus
QTTIHDIILECVYCK

SEQ ID NO: 2501 - protein, 15 aa, Human papillomavirus
LKFYSKISEYRHYCY

SEQ ID NO: 2502 - protein, 15 aa, Human papillomavirus
GTTLEQQYNKPLCDL

SEQ ID NO: 2503 - protein, 15 aa, Human papillomavirus
FRDLCIVYRDGNPYA

SEQ ID NO: 2504 - protein, 15 aa, Human papillomavirus
GNPYAVCDKCLKFYS

SEQ ID NO: 2505 - protein, 15 aa, Human papillomavirus
REVYDFAFRDLCIVY

SEQ ID NO: 2506 - protein, 15 aa, Human papillomavirus
DLLIRCINCQKPLCP

SEQ ID NO: 2507 - protein, 15 aa, Human papillomavirus
LKFYSKISEYRHYCY

SEQ ID NO: 2508 - protein, 15 aa, Human papillomavirus
DKCLKFYSKISEYRH

SEQ ID NO: 2509 - protein, 15 aa, Human papillomavirus
RREVYDFAFRDLCIV

SEQ ID NO: 2510 - protein, 15 aa, Human papillomavirus
IRCINCQKPLCPEEK

SEQ ID NO: 2511 - protein, 15 aa, Human papillomavirus
LDKKQRFHNIRGRWT

SEQ ID NO: 2512 - protein, 15 aa, Human papillomavirus
LECVYCKQQLLRREV

SEQ ID NO: 2513 - protein, 98 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 2514 - protein, 11 aa, Human papillomavirus
MHGDTPTLHEY

SEQ ID NO: 2515 - protein, 10 aa, Human papillomavirus
HGDTPTLHEY

SEQ ID NO: 2516 - protein, 10 aa, Human papillomavirus
QPETTDLYCY

SEQ ID NO: 2517 - protein, 10 aa, Human papillomavirus
DLQPETTDLY

SEQ ID NO: 2518 - protein, 9 aa, Human papillomavirus
QAEPDRAHY

SEQ ID NO: 2519 - protein, 10 aa, Human papillomavirus
DLLMGTLGIV

SEQ ID NO: 2520 - protein, 10 aa, Human papillomavirus
TLEDLLMGTL

SEQ ID NO: 2521 - protein, 9 aa, Human papillomavirus
LLMGTLGIV

SEQ ID NO: 2522 - protein, 9 aa, Human papillomavirus
TLHEYMLDL

SEQ ID NO: 2523 - protein, 9 aa, Human papillomavirus
DLQPETTDL

SEQ ID NO: 2524 - protein, 10 aa, Human papillomavirus
TLRLCVQSTH

SEQ ID NO: 2525 - protein, 10 aa, Human papillomavirus
GIVCPICSQK

SEQ ID NO: 2526 - protein, 10 aa, Human papillomavirus
DLQPETTDLY

SEQ ID NO: 2527 - protein, 10 aa, Human papillomavirus
LLMGTLGIVC

SEQ ID NO: 2528 - protein, 9 aa, Human papillomavirus
IVCPICSQK

SEQ ID NO: 2529 - protein, 10 aa, Human papillomavirus
CVQSTHVDIR

SEQ ID NO: 2530 - protein, 10 aa, Human papillomavirus
GIVCPICSQK

SEQ ID NO: 2531 - protein, 10 aa, Human papillomavirus
SSEEEDEIDG

SEQ ID NO: 2532 - protein, 9 aa, Human papillomavirus
IVCPICSQK

SEQ ID NO: 2533 - protein, 9 aa, Human papillomavirus
STLRLCVQS

SEQ ID NO: 2534 - protein, 10 aa, Human papillomavirus
TFCCKCDSTL

SEQ ID NO: 2535 - protein, 10 aa, Human papillomavirus
VDIRTLEDLL

SEQ ID NO: 2536 - protein, 10 aa, Human papillomavirus
AEPDRAHYNI

SEQ ID NO: 2537 - protein, 9 aa, Human papillomavirus
TDLYCYEQL

SEQ ID NO: 2538 - protein, 9 aa, Human papillomavirus
GTLGIVCPI

SEQ ID NO: 2539 - protein, 10 aa, Human papillomavirus
DTPTLHEYML

SEQ ID NO: 2540 - protein, 10 aa, Human papillomavirus
HVDIRTLEDL

SEQ ID NO: 2541 - protein, 10 aa, Human papillomavirus
DRAHYNIVTF

SEQ ID NO: 2542 - protein, 9 aa, Human papillomavirus
STHVDIRTL

SEQ ID NO: 2543 - protein, 9 aa, Human papillomavirus
ETTDLYCYE

SEQ ID NO: 2544 - protein, 11 aa, Human papillomavirus
TFCCKCDSTLR

SEQ ID NO: 2545 - protein, 11 aa, Human papillomavirus
ETTDLYCYEQL

SEQ ID NO: 2546 - protein, 10 aa, Human papillomavirus
CVQSTHVDIR

SEQ ID NO: 2547 - protein, 9 aa, Human papillomavirus
IVCPICSQK

SEQ ID NO: 2548 - protein, 9 aa, Human papillomavirus
PAGQAEPDR

SEQ ID NO: 2549 - protein, 10 aa, Human papillomavirus
EPDRAHYNIV

SEQ ID NO: 2550 - protein, 10 aa, Human papillomavirus
GPAGQAEPDR

SEQ ID NO: 2551 - protein, 10 aa, Human papillomavirus
CCKCDSTLRL

SEQ ID NO: 2552 - protein, 9 aa, Human papillomavirus
TPTLHEYML

SEQ ID NO: 2553 - protein, 9 aa, Human papillomavirus
EIDGPAGQA

SEQ ID NO: 2554 - protein, 9 aa, Human papillomavirus
DIRTLEDLL

SEQ ID NO: 2555 - protein, 9 aa, Human papillomavirus
TLHEYMLDL

SEQ ID NO: 2556 - protein, 9 aa, Human papillomavirus
TPTLHEYML

SEQ ID NO: 2557 - protein, 9 aa, Human papillomavirus
DLQPETTDL

SEQ ID NO: 2558 - protein, 8 aa, Human papillomavirus
CCKCDSTL

SEQ ID NO: 2559 - protein, 10 aa, Human papillomavirus
DLQPETTDLY

SEQ ID NO: 2560 - protein, 10 aa, Human papillomavirus
GQAEPDRAHY

SEQ ID NO: 2561 - protein, 10 aa, Human papillomavirus
TLRLCVQSTH

SEQ ID NO: 2562 - protein, 10 aa, Human papillomavirus
LLMGTLGIVC

SEQ ID NO: 2563 - protein, 9 aa, Human papillomavirus
LQPETTDLY

SEQ ID NO: 2564 - protein, 9 aa, Human papillomavirus
LEDLLMGTL

SEQ ID NO: 2565 - protein, 9 aa, Human papillomavirus
PETTDLYCY

SEQ ID NO: 2566 - protein, 9 aa, Human papillomavirus
DEIDGPAGQ

SEQ ID NO: 2567 - protein, 9 aa, Human papillomavirus
DIRTLEDLL

SEQ ID NO: 2568 - protein, 8 aa, Human papillomavirus
AEPDRAHY

SEQ ID NO: 2569 - protein, 10 aa, Human papillomavirus
DRAHYNIVTF

SEQ ID NO: 2570 - protein, 10 aa, Human papillomavirus
LDLQPETTDL

SEQ ID NO: 2571 - protein, 9 aa, Human papillomavirus
LRLCVQSTH

SEQ ID NO: 2572 - protein, 9 aa, Human papillomavirus
IRTLEDLLM

SEQ ID NO: 2573 - protein, 9 aa, Human papillomavirus
RAHYNIVTF

SEQ ID NO: 2574 - protein, 10 aa, Human papillomavirus
QPETTDLYCY

SEQ ID NO: 2575 - protein, 9 aa, Human papillomavirus
TPTLHEYML

SEQ ID NO: 2576 - protein, 9 aa, Human papillomavirus
EPDRAHYNI

SEQ ID NO: 2577 - protein, 9 aa, Human papillomavirus
FCCKCDSTL

SEQ ID NO: 2578 - protein, 9 aa, Human papillomavirus
LEDLLMGTL

SEQ ID NO: 2579 - protein, 10 aa, Human papillomavirus
QSTHVDIRTL

SEQ ID NO: 2580 - protein, 9 aa, Human papillomavirus
RAHYNIVTF

SEQ ID NO: 2581 - protein, 9 aa, Human papillomavirus
DSSEEEDEI

SEQ ID NO: 2582 - protein, 9 aa, Human papillomavirus
GTLGIVCPI

SEQ ID NO: 2583 - protein, 9 aa, Human papillomavirus
DTPTLHEYM

SEQ ID NO: 2584 - protein, 15 aa, Human papillomavirus
MGTLGIVCPICSQKP

SEQ ID NO: 2585 - protein, 15 aa, Human papillomavirus
HVDIRTLEDLLMGTL

SEQ ID NO: 2586 - protein, 15 aa, Human papillomavirus
DLLMGTLGIVCPICS

SEQ ID NO: 2587 - protein, 15 aa, Human papillomavirus
IRTLEDLLMGTLGIV

SEQ ID NO: 2588 - protein, 15 aa, Human papillomavirus
TLEDLLMGTLGIVCP

SEQ ID NO: 2589 - protein, 15 aa, Human papillomavirus
TFCCKCDSTLRLCVQ

SEQ ID NO: 2590 - protein, 15 aa, Human papillomavirus
MLDLQPETTDLYCYE

SEQ ID NO: 2591 - protein, 15 aa, Human papillomavirus
TTDLYCYEQLNDSSE

SEQ ID NO: 2592 - protein, 15 aa, Human papillomavirus
IRTLEDLLMGTLGIV

SEQ ID NO: 2593 - protein, 15 aa, Human papillomavirus
LHEYMLDLQPETTDL

SEQ ID NO: 2594 - protein, 15 aa, Human papillomavirus
LHEYMLDLQPETTDL

SEQ ID NO: 2595 - protein, 15 aa, Human papillomavirus
TDLYCYEQLNDSSEE

SEQ ID NO: 2596 - protein, 15 aa, Human papillomavirus
RAHYNIVTFCCKCDS

SEQ ID NO: 2597 - protein, 15 aa, Human papillomavirus
HEYMLDLQPETTDLY

SEQ ID NO: 2598 - protein, 15 aa, Human papillomavirus
MLDLQPETTDLYCYE

SEQ ID NO: 2599 - protein, 15 aa, Human papillomavirus
IVTFCCKCDSTLRLC

SEQ ID NO: 2600 - protein, 15 aa, Human papillomavirus
MLDLQPETTDLYCYE

SEQ ID NO: 2601 - protein, 15 aa, Human papillomavirus
THVDIRTLEDLLMGT

SEQ ID NO: 2602 - protein, 15 aa, Human papillomavirus
HVDIRTLEDLLMGTL

SEQ ID NO: 2603 - protein, 15 aa, Human papillomavirus
YEQLNDSSEEEDEID

SEQ ID NO: 2604 - protein, 15 aa, Human papillomavirus
YNIVTFCCKCDSTLR

SEQ ID NO: 2605 - protein, 15 aa, Human papillomavirus
DLLMGTLGIVCPICS

SEQ ID NO: 2606 - protein, 15 aa, Human papillomavirus
MGTLGIVCPICSQKP

SEQ ID NO: 2607 - protein, 15 aa, Human papillomavirus
TDLYCYEQLNDSSEE

SEQ ID NO: 2608 - protein, 15 aa, Human papillomavirus
LYCYEQLNDSSEEED

SEQ ID NO: 2609 - protein, 15 aa, Human papillomavirus
TTDLYCYEQLNDSSE

SEQ ID NO: 2610 - protein, 15 aa, Human papillomavirus
TPTLHEYMLDLQPET

SEQ ID NO: 2611 - protein, 15 aa, Human papillomavirus
EDLLMGTLGIVCPIC

SEQ ID NO: 2612 - protein, 15 aa, Human papillomavirus
MGTLGIVCPICSQKP

SEQ ID NO: 2613 - protein, 15 aa, Human papillomavirus
HYNIVTFCCKCDSTL

(19) United States
(12) Patent Application Publication
(10) Pub. No.: US 2023/0002730 A1
(43) Pub. Date: Jan. 5, 2023

(54) IMPROVED TARGETED T-CELL THERAPY

(71) Applicant: CHILDREN'S NATIONAL MEDICAL CENTER, Washington, DC (US)

(72) Inventors: Catherine Mary Bollard, Bethesda, MD (US); Conrad Russell Y. Cruz, Bethesda, MD (US); Patrick Hanley, Washington, DC (US)

(73) Assignee: CHILDREN'S NATIONAL MEDICAL CENTER, Washington, DC (US)

(21) Appl. No.: 17/056,714

(22) PCT Filed: May 20, 2019
(86) PCT No.: PCT/US2019/033182

§ 371 Date: Nov. 18, 2020

Related U.S. Application Data
(60) Provisional application No. 62/673,745, filed on May 18, 2018.

(51) Int. Cl. C12N 5/0783; A61K 35/17; C07K 14/47

(52) U.S. Cl. CPC C12N 5/0636; G01N 33/68; C07K 14/4748; A61K 35/17

(57) ABSTRACT

Disclosed are compositions of cells, libraries of such cells and methods of making T cell populations for treatment of disorders such as cancer and viral infections. T cell composition comprise cell subpopulations stimulated, in some embodiments, with FRAME, survivin and/or WT1.

### RELATED APPLICATIONS

This application claims the benefit of provisional U.S. Application No. 62/673,745, filed, May 18, 2018, the entirety of which is hereby incorporated by reference for all purposes.

### FIELD OF THE INVENTION

The present invention provides improved non-engineered adoptive T-cell compositions, therapies, and processes of manufacture that are tailored to the specific antigenic expression of a patient's tumor, allowing for changes to the T-cell composition that is administered in response to changes in tumor expression over time based on either pressure from antineoplastic therapy or natural heterogeneous selection. In certain embodiments, the invention includes non-engineered adoptive T-cell compositions and their use and manufacture for the treatment of hematological malignancies or solid tumors. The present invention also extends to methods of manufacturing such non-engineered adoptive T-cell compositions and the generation of single antigen targeting T-cell banks from healthy donors that provide for improved personalized T-cell therapy.

### BACKGROUND OF THE INVENTION

Adoptive immunotherapy is an approach used to bolster the ability of the immune system to fight diseases, such as tumor and viral infections. According to this approach, T cells are collected from a patient or donor, stimulated in the presence of antigen presenting cells bearing tumor or viral-associated antigens, and then expanded ex vivo. These non-engineered T cells are given to the patient to help the immune system fight the disease.

Activated T-cell approaches have been reported since the early 2000's. Efforts began with the use of autologous blood that was activated by exposure ex vivo to viral antigens, typically in the context of treatment of patients who had undergone hematopoietic stem cell therapy and needed additional immune capacity, especially to fight viral diseases such as Epstein-Barr virus, cytomegalovirus, adenovirus and herpes simplex virus, as well as respiratory viral infections from RSV (respiratory syncytial virus), parainfluenza, and influenza. The efforts later expanded into allogeneic approaches for stem cell therapy patients followed by various approaches to attempt to use tumor associated antigen activated autologous or allogeneic blood sources. This approach has been shown to have some success clinically in the viral and tumor settings by multiple groups (Hague et al. Blood (2007) 110(4):1123-1131 and Leen et al. Blood (2013) 121(26):5113-5123). Blood from both naïve and non-naïve donors has been evaluated. A number of groups have also shown clinical success in the viral and tumor setting using a naïve T cell donor source with both single and multiple antigens (Park et al. Blood (2006) 108:1770-3; Hanley et al. Blood (2009) 114:1958-67; Jedema et al. Haematologica (2011) 96:1204-112).

There are a number of ongoing human clinical trials evaluating a range of T-cell strategies. These include the RESOLVE trial, which is administering allogeneic T-cells to treat leukemia patients; the REST trial, which is evaluating autologous and allogeneic tumor associated antigen lymphocytes for the treatment of solid tumors; the TACTAM trial, which is administering autologous T-cells to treat multiple myeloma patients; the ADSPAM trial, which is administering allogeneic T-cells to treat AML and MDS patients; the MUSTAT trial, which is evaluating autologous and allogeneic T-cells primed with CMV, EBV, and/or adenovirus; the CHAPS trial, which is evaluating allogeneic viral antigen primed T-cells; the NATS trial, which is evaluating a multivalent 6-viral antigen approach for transplant patients; the HXTC and RESIST trials, which are evaluating autologous HIV activated T-cells; the ACTCAT2 trial, which is evaluating cord blood primed with viral antigens; and the CHEERS trial, which is evaluating cord blood activated with multiple viral antigens.

Recent strategies have been developed to generate activated T-cells targeting multiple potential antigens in a single T-cell product. In particular, approaches to generate multi-antigen specific T-cells have focused on priming and activating T-cells with multiple targeted antigen overlapping peptide libraries (a “PepMix™”), for example multiple libraries of 15mer peptides overlapping by 11 amino acids spanning the whole amino acid sequence of several target antigens (see for example commercially available overlapping peptide library products from JPT Technologies or Miltenyi). For example, WO 2016/154112, assigned to Children's National Medical Center, describes the generation of cytotoxic T-lymphocytes (CTLs) reactive against multiple tumor antigens simultaneously by stimulation with dendritic cells pulsed with mixtures of overlapping peptide libraries spanning the antigens of interest as a stimulus in the presence of a cytokine cocktail.

The overlapping peptide libraries, however, include some peptide segments that are antigenic and others that are not. In these processes, the individual overlapping peptide libraries of the selected antigens are generally mixed in equal amounts regardless of the molecular weight of the protein antigen to create the mastermix for T-cell priming, and single batches of T-cells are exposed to the multi-antigen overlapping peptide libraries. While this approach does provide the potential for a “universal” protocol to the generation of multi-TAA-specific T-cells, the mastermix of overlapping peptide libraries, however, may not be a good match for the patient's specific tumor expression profile, which decreases the potential efficacy of the therapy. Further, since the peptides have different molecular weights, using the same weight amount of the overlapping peptide library for each antigenic protein in the cocktail results in the use of fewer segment duplicates in the libraries of the higher molecular weight proteins. Also, it is somewhat random how many active epitopes each protein has so that one overlapping peptide library might contain more immunogenic epitopes than another overlapping peptide library, regardless of molecular weight. Additional causes include the use of nonimmunogenic antigens, which can elicit tolerance or introduce potential avenues for autoimmunity if other unnecessary peptides are cross reactive. Consequently, the approach leads to large variability of primed and activated T-cells to each particular antigen within each generated T-cell product, which may not be reflective of the tumor antigen profile of any particular patient's tumor, and issues of lack of optimal targeting and efficacy remain.

While progress has been made in adoptive T-cell therapy, given its importance to tumor therapy, there is a strong need to improve the efficiency and outcomes of the therapy. As one example, there remains a need to improve adoptive immunotherapy for the treatment of disorders, including hematological malignancies and other tumors.

### SUMMARY OF THE INVENTION

Provided herein are improved compositions and methods for use in adoptive T-cell therapy to treat human tumors. Non-engineered T-cell compositions that include in the same dosage form a multiplicity of T-cell subpopulations are provided for administration to a human patient with a tumor, wherein each T-cell subpopulation is specific for a single tumor-associated antigen (TAA), and the T-cell subpopulations that comprise the T-cell composition for administration are chosen specifically based on the TAA expression profile of the patient's tumor. By using separate activated T-cell subpopulations to form the T-cell composition for administration, the T-cell composition as a whole includes individual T-cell subpopulations targeting specific TAAs, resulting in a highly consistent and activated T-cell composition capable of targeting multiple TAAs. Furthermore, by selecting the T-cell subpopulations based on the patient's TAA expression profile, a highly targeted T-cell composition is administered having the potential for increased efficacy, increased level of consistency and characterization, and decreased potential for generating off-target effects from the use of T-cells which target antigens not expressed by the patient's tumor. All of these factors are very important to the approval process of products developed by this approach. The resulting T-cell therapeutic composition is referred to herein as a “MUltiple Single Tumor ANtiGen” T-cell composition or “MUSTANG” composition.

Prior strategies employed in the ex vivo expansion of single populations of T-cells by repeated stimulation using multi-TAA strategies, for example multi-TAA overlapping peptide libraries (as exemplified in, e.g., FIG. 1)—while promising—result in highly heterogeneous T-cell products with variable T-cell subpopulations specific for the individual antigens of the multi-TAA mixture. These T-cell compositions can vary widely from one sample or batch to another due to the great variability in multi-TAA activation, and in a number of cases, the resultant T-cell composition may have little or no activity to one or more of the targeted TAAs. For example, Weber et al. describe the generation of multi-TAA specific T-cell compositions wherein allogeneic healthy donor peripheral blood mononuclear cells were stimulated ex vivo with dendritic cells pulsed with complete peptide libraries of five TAAs—proteinase 3 (Pr3), Wilms tumor gene 1 (WT1), human neutrophil elastase (NE), preferentially expressed antigen in melanoma (PRAME), and melanoma-associated antigen A3 (MAGE-A3), antigens frequently expressed on myeloid leukemias. Activity of the ex vivo expanded T-cell compositions as measured by IFNγ-ELISpot assay showed that 10% of generated T-cell compositions recognized only 1 of the 5 TAAs, 40% recognized only 2 of the 5 TAAs, 30% recognized only 3 of the 5 TAAs, and only 20% of the generated T-cell compositions recognized 4 of 5 targeted TAAs. Accordingly, none of the generated T-cell compositions recognized all 5 of the targeted TAAs, while 50% of the generated T-cell compositions using a multi-TAA overlapping peptide library approach failed to recognize two or more of the targeted TAAs. See Weber et al., Generation of multi-leukemia antigen-specific T cells to enhance the graft-versus-leukemia effect after allogeneic stem cell transplant. Leukemia (2013) 27:1538-1547 (FIG. 1d, le and if of publication, FIGS. 4, 5 and 6 herein); see also FIGS. 4, 5 and 6 herein and Example 2, FIG. 3 (showing the variability of products generated using pooled, multi-TAA overlapping peptide libraries).

Unlike the random T-cell compositions derived by the use of pooled, multi-TAA overlapping peptide libraries which may result in considerable variability and, in some instances, no activity against one or more targeted TAAs despite their inclusion in the master mix, the present invention avoids the significant variability of these compositions. This invention, a MUSTANG composition, and its use and manufacture, differs from the prior art in that the T-cells are not, as a group, exposed to a mastermix of peptide fragments or overlapping peptide libraries from multiple TAAs. Instead, T-cell subpopulations are each exposed to either an overlapping peptide library from a single TAA, an overlapping peptide library plus one or more selected immunogenic peptides from a single TAA, including and perhaps substantially comprised of selected cell donor HLA-restricted peptide immunogenic epitope(s) of the TAA, or a specially selected mix of one or more immunogenic peptides from a single TAA, including and perhaps substantially comprised of selected cell donor HLA-restricted peptide immunogenic epitope(s) of the TAA. The therapeutic dosage form of the MUSTANG includes more than one, for example two, three, four, or five or more T-cell subpopulations, wherein each T-cell subpopulation is specific for a single TAA; that is, the separate T-cell subpopulations that comprise the MUSTANG are each primed to a single tumor antigen, for example each T-cell subpopulation is capable of recognizing one TAA. The particular T-cell subpopulations that make up the MUSTANG composition target TAAs that are representative of the TAA expression profile of a patient's tumor. In some embodiments, the percentage of each specific TAA-targeting T-cell subpopulation in the MUSTANG composition correlates with the tumor-associated antigen expression profile of the tumor in the patient receiving the treatment. In some embodiments, the percentage of each specific TAA-targeting T-cell subpopulation in the MUSTANG composition is measured by cell number of the T-cell subpopulation. In some embodiment, the percentage of each specific TAA-targeting T-cell subpopulation in the MUSTANG composition is measured by the activity of the T-cell subpopulation.

The T-cell subpopulations that comprise the MUSTANG composition each target a single TAA. The generation of each T-cell subpopulation can be accomplished through the ex vivo priming and activation of the T-cell subpopulation to one or more peptides from a single TAA. In certain embodiments, if more than one peptide from a single, targeted tumor antigen is used, the peptide segments can be generated by making overlapping peptide fragments of the tumor antigen, as provided for example in commercially available overlapping peptide libraries or “PepMixes™.” Examples include commercially available overlapping peptide libraries from JPT Technologies or Miltenyi. In particular embodiments, the peptides of the overlapping peptide library are 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and there is overlap of 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length. In certain embodiments, if more than one peptide from a single, targeted tumor antigen is used, the peptide segments can be generated by making overlapping peptide fragments of the tumor antigen, as provided for example in commercially available overlapping peptide fragments, and further enriched with certain antigenic epitopes of the targeted TAA that are active through specific cell donor HLA alleles, for example, a single specific HLA-restricted epitope or multiple specific HLA-restricted epitopes of the TAA. In certain embodiments, if more than one peptide from a single, targeted tumor antigen is used, the peptide segments can be selected from certain antigenic epitopes of the targeted TAA that are active through specific HLA alleles, for example, a single specific HLA-restricted epitope or multiple specific HLA-restricted epitopes of the TAA.

In some embodiments, the T-cell subpopulation is primed with a single TAA peptide mix, wherein the peptide mix comprises antigenic epitopes derived from a TAA based on one or more of the donor's HLA phenotypes, for example, the peptides are restricted through one or more of the cell donor's HLA alleles such as, but not limited to, HLA-A, HLA-B, and HLA-DR. By including specifically selected donor HLA-restricted peptides from a single TAA in the peptide mix for priming and expanding each T-cell subpopulation, a T-cell subpopulation can be generated that provides greater TAA targeted activity through one or more donor HLA alleles, improving potential efficacy of the T-cell subpopulation for patients that share at least one HLA allele with the donor. In addition, by generating a T-cell subpopulation with TAA targeted activity through more than one donor HLA allele, a single donor T-cell subpopulation may be included in a MUSTANG composition for multiple recipients with different HLA profiles by matching one or more donor HLA alleles showing TAA-activity (see, for example, Example 5 and FIG. 9). In some embodiments, the TAA peptides used to prime and expand a T-cell subpopulation are generated based on a cell donor's HLA profile, wherein the peptides are HLA-restricted epitopes specific to at least one or more of a donor's HLA-A alleles, HLA-B alleles, or HLA-DR alleles, or a combination thereof. In some embodiments, the HLA-A alleles are selected from a group comprising HLA-A*01, HLA-A*02:01, HLA-A*03, HLA-A*11:01, HLA-A*24:02, HLA-A*26, and HLA-A*68:01. In some embodiments, the HLA-B alleles are selected from a group comprising HLA-B*07:02, HLA-B*08, HLA-B*15:01 (B62), HLA-B*18, HLA-B*27:05, HLA-B*35:01, and HLA-B*58:02. In some embodiments, the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, and HLA-DRB1*1501 (DR2b). In some embodiments, the mastermix of peptides includes both an overlapping peptide library and specifically selected HLA-restricted peptides generated by determining the HLA profile of the donor source.

This improved T-cell therapy can be used to treat hematological or solid tumors. In certain nonlimiting examples, the tumor is a leukemia, lymphoma, or myeloma, including but not limited to acute myeloid leukemia, acute lymphoblastic leukemia, chronic lymphoblastic leukemia, multiple myeloma, or a solid tumor such as breast cancer, prostate cancer, melanoma, sarcoma, carcinoma, osteosarcoma, neuroblastoma, pancreatic, or lung, including but not limited to small cell and non-small cell lung cancer, Wilms tumor, rhabdomyosarcoma, and Ewing sarcoma. In certain embodiments, the tumor is relapsed, refractory to standard of care treatment, or has become resistant over time to other anti-tumor approaches. In some embodiments, the tumor is a relapsed or refractory leukemia, lymphoma, or myeloma. In some embodiments, the tumor is a relapsed or refractory solid tumor. Alternatively, the cell compositions described herein can be administered to a patient with a viral-induced tumor, for example but not limited to: hepatitis B or hepatitis C virus induced cirrhosis or liver cancer; human papillomavirus (HPV) induced cervical, anogenital, and head and neck cancers; Epstein-Barr virus (EBV) induced Burkitt's lymphoma and nasopharyngeal carcinoma; herpes virus (HHV) associated Kaposi's sarcoma; human T-cell lymphotropic virus associated adult T-cell leukemia; and HIV-related cancers.

Importantly, this advantageous T-cell therapy can be optimized for personal efficacy in the patient by testing each T-cell subpopulation for activity against and responsiveness to the patient's tumor. As discussed in the Background, one of the problems associated with administration of a T-cell population primed and activated with a mastermix of peptides or overlapping peptide libraries from multiple tumor antigens is that it may include a significant number of T-cells that do not generate a response against a patient's tumor. Therefore, in some embodiments, the invention includes priming and activating T-cell subpopulations for inclusion in the MUSTANG composition which have been primed and activated with specific TAAs based on the tumor-type of the patient. In some embodiments, epitopes expressed by a patient's tumor are first identified and T-cell subpopulations primed with peptides to those epitopes are included in the MUSTANG composition. In an alternative embodiment, specific epitopes expressed by a patient's tumor are first identified and peptides specific to those epitopes are synthesized and are used to prime and activate a T-cell subpopulation. By using or including specifically expressed patient tumor epitopes in a peptide mixture to prime and activate specific T-cell subpopulations, the peptide mixture for the specific TAA can be optimized to increase the likelihood of generating cytotoxic T lymphocytes active against the patient's tumor through shared HLA alleles with the donor, and the ability of the T-cell subpopulation to recognize the TAA can be confirmed ex vivo. In some embodiments, the generated T-cell subpopulation can be tested for activity against the patient's tumor ex vivo to confirm a robust response. This can be repeated for some or all of the remaining T-cell subpopulations comprising the MUSTANG composition until it is confirmed that one, some or all of the T-cell subpopulations are primed and activated against the targeted TAAs of the patient.

T-cell subpopulations used in the MUSTANG composition are capable of recognizing one epitope, two epitopes, three epitopes, or more than three epitopes of a single TAA. In some embodiments, the MUSTANG composition includes more than one T-cell subpopulation targeting the same TAA, wherein each T-cell subpopulation is capable of recognizing discrete and separate epitopes within the same TAA.

Each T-cell subpopulation of the MUSTANG composition is generated to be specific to a single TAA. TAAs for targeting by the T-cell subpopulations may include any TAA expressed by the tumor, for example, an oncofetal, an oncoviral, overexpressed/accumulated, cancer-testis, lineage-restricted, mutated, post-translationally altered, or idiotypic antigen. Although they are preferentially expressed by tumor cells, TAAs are oftentimes found in normal tissues. However, their expression differs from that of normal tissues by their degree of expression in the tumor, alterations in their protein structure in comparison with their normal counterparts or by their aberrant subcellular localization within malignant or tumor cells. Non-limiting examples of TAAs, in certain embodiments, for targeting may be selected from one or more peptide segment(s), overlapping peptide libraries, or selected epitope(s) of Carcinoembryonic antigen (CEA), immature laminin receptor, and tumor-associated glycoprotein (TAG) 72, human papilloma virus (HPV) E6 and E7, Epstein-Barr Virus (EBV) Epsteing-Barr nuclear antigen (EBNA), latent membrane protein (LMP) 1 and 2, BING-4, calcium-activated chloride channel (CLCA) 2, Cyclin A₁, Cyclin B₁, 9D7, epithelial cell adhesion molecule (Ep-Cam), EphA3, Her2/neu, telomerase, mesothelin, stomach cancer-associated protein tyrosine phosphatase 1 (SAP-1), survivin, b melanoma antigen (BAGE) family, cancer-associated gene (CAGE) family, G antigen (GAGE) family, melanoma antigen (MAGE) family, sarcoma antigen (SAGE) family, X antigen (XAGE) family, CT9, CT10, NY-ESO-1, L antigen (LAGE) 1, Melanoma antigen preferentially expressed in tumors (PRAME), synovial sarcoma X (SSX) 2, melanoma antigen recognized by T cells-1/2 (Melan-A/MART-1/2), Gp100/pmel17, tyrosinase, tyrosine-related protein (TRP) 1 and 2, P.polypeptide, melanocortin 1 receptor (MC1R), prostate-specific antigen, β-catenin, breast cancer antigen (BRCA) 1/2, cyclin-dependent kinase (CDK) 4, chronic myelogenous leukemia antigen (CML) 66, fibronectin, MART-2, p53, Ras, TGF-βRII, mucin (MUC) 1, immunoglobulin (Ig) and T cell receptor (TCR). In some embodiments, the tumor antigen is a neoantigen. In some embodiments, the neoantigen is a mutated form of an endogenous protein derived through a single point mutation, a deletion, an insertion, a frameshift mutation, a fusion, mis-spliced peptide, or intron translation.

In some embodiments, the MUSTANG composition includes one or more T-cell subpopulations targeting WT1, PRAME, Survivin, NY-ESO-1, MAGE-A3, MAGE-A4, Pr3, Cyclin A₁, SSX2, Neutrophil Elastase (NE), HPV E6. HPV E7, EBV LMP1, EBV LMP2, EBV EBNA1, and EBV EBNA2. In some embodiments, the MUSTANG composition includes one or more T-cell subpopulations targeting WT1, PRAME, and Survivin. In some embodiments, the MUSTANG composition consists of individual T-cell subpopulations targeting WT1, PRAME, and Survivin, wherein the peptides used to generate the T-cell subpopulations are overlapping peptide libraries. In some embodiments, the MUSTANG composition consists of individual T-cell subpopulations targeting WT1, PRAME, and Survivin, wherein the peptides used to generate the T-cell subpopulations are overlapping peptide libraries that have been further enriched with one or more specific known or identified epitopes expressed by the patient's tumor. In some embodiments, the MUSTANG composition consists of individual T-cell subpopulations targeting WT1, PRAME, and Survivin, wherein the peptides used to generate the T-cell subpopulations are specifically selected epitopes of the TAA that are HLA-restricted based on a cell donor's HLA type. In some embodiments, the HLA-restricted epitopes are specific to at least one or more of a cell donor's HLA-A alleles, HLA-B alleles, or HLA-DR alleles. In some embodiments, the HLA-A alleles are selected from a group comprising HLA-A*01, HLA-A*02:01, HLA-A*03, HLA-A*11:01, HLA-A*24:02, HLA-A*26, or HLA-A*68:01. In some embodiments, the HLA-B alleles are selected from a group comprising HLA-B*07:02, HLA-B*08, HLA-B*15:01 (B62), HLA-B*18, HLA-B*27:05, HLA-B*35:01, or HLA-B*58:02. In some embodiments, the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b).

In some embodiments, a sample of the patient's tumor is taken by biopsy, blood sample, or other isolation, and a profile of associated antigenic proteins expressed in the tumor is identified and quantified, and the T-cell subpopulations of the MUSTANG composition target one or more of the expressed tumorigenic antigens. In another embodiment, an epitope profile of expressed antigenic proteins is identified, and the T-cell subpopulations of the MUSTANG composition target one or more of the identified epitopes. In some embodiments, the selected antigenic proteins are not overexpressed self-proteins which have not been mutated, rearranged or otherwise altered over the normal sequence and conformation.

In some embodiments, the T-cell subpopulations for inclusion in the MUSTANG composition are autologously derived from the patient. In some embodiments, the T-cell subpopulations for inclusion in the MUSTANG composition are derived from an allogeneic donor, for example, from the peripheral blood, apheresis product, or bone marrow from a naïve, healthy donor. In some embodiments, the T-cell subpopulations for inclusion in the MUSTANG composition are derived from cord blood.

In one aspect, the invention further includes a bank of individual T-cell subpopulations, and methods of manufacturing a bank of individual T-cell subpopulations with an associated phenotypic characteristic database. The bank includes individual T-cell subpopulations which have been primed and activated to a specific, single TAA. The T-cell subpopulations are derived from an allogeneic donor source, for example, the peripheral blood, apheresis product or bone marrow from a naïve, healthy donor and/or cord blood sample. The T-cell subpopulations are HLA-typed and the donor source recorded. The T-cell subpopulations' antigenic recognition response is verified and characterized, for example, via ELISPOT IFN-γ assay, IL-2 assay, TNF-α assay, or multimer assay to quantify the activity of the T-cell population against the specific, targeted TAA. Alternatively, the diversity of T-cell receptor (TCR) α-chain and β-chain repertoire can be characterized, for example, using TCR ligation-anchored-magnetically captured PCR (TCR-LA-MC PCR) (see, e.g., Ruggiero et al., High-resolution analysis of the human T-cell receptor repertoire, Nat. Cumm. 2015 6:8081) or other appropriate characterization techniques. Furthermore, the T-cell subpopulations' antigenic recognition response is further characterized through its corresponding HLA-allele, for example through an HLA restriction assay. The T-cell subpopulations can be cryopreserved and stored. In some embodiments, the T-cell subpopulations are stored based on the donor source. In some embodiments, the T-cell subpopulations are stored by TAA specificity. In some embodiments, the T-cell subpopulations are stored by human leukocyte antigen (HLA) subtype and restrictions.

By characterizing each T-cell subpopulations' reactivity and corresponding HLA-allele, a MUSTANG composition can be optimized for each patient based on specific T-cell subpopulation reactivity and HLA matching, providing a highly personalized T-cell therapy. Accordingly, if a patient has a tumor that expresses one epitope of a TAA but not another, or if one epitope of a TAA invokes a greater T-cell response, that T-cell subpopulation can be taken from the bank and used in the MUSTANG composition. In this way, the T-cell therapy can be tailored to evoke a maximal response against the patient's tumor.

This invention thus acknowledges and accounts for the fact that T-cells from various donors may have variable activity against the same tumor associated antigen, or even the same epitope, generating T-cell responses with varying efficiency. This fact is taken into account when producing the comprehensive bank of a wide variety of allogeneic activated T-cells for personalized T-cell therapeutic composition of the invention. Derived T-cell subpopulations having shared HLA-alleles that exhibit strong activity to an epitope or tumor antigen expressed in the patient's tumor can be selected from the bank for inclusion in the MUSTANG composition. In some embodiments, one or more of the T-cell subpopulations for consideration for inclusion in the MUSTANG composition are tested against cells from the patient's tumor prior to administration in vivo by exposing the patient's tumor cells in vitro to the one or more T-cell subpopulations and determining the T-cell subpopulation's ability to lyse the tumor cell. In this way, the probability of the MUSTANG composition inducing a therapeutic response upon administration to the patient is greatly enhanced.

In one aspect, provided herein is a method of treating a patient with a tumor comprising:
i) determining the HLA subtype of the patient;
ii) diagnosing the tumor type of the patient;
iii) identifying two or more tumor associated antigens associated with the tumor type for targeting with TAA-specific T-cell subpopulations;
iv) selecting one banked T-cell subpopulation having the highest activity against each targeted TAA through one or more HLA-alleles shared between the patient and the T-cell subpopulations, wherein each T-cell subpopulation is specific for a single tumor associated antigen, wherein each of the T-cell subpopulations is specific for a different tumor associated antigen, wherein each of the T-cell subpopulations are primed and expanded separately from each other, wherein each of the T-cell subpopulations are primed and expanded ex vivo;
v) combining each selected banked T-cell subpopulation to create a MUSTANG composition; and,
vi) administering an effective amount of the MUSTANG composition to the patient.

In one aspect, provided herein is a method of treating a patient with a tumor comprising:
i) determining the HLA subtype of the patient;
ii) determining the TAA expression profile of the patient's tumor;
iii) identifying two or more tumor associated antigens expressed by the patient's tumor for targeting with TAA-specific T-cell subpopulations;
iv) selecting one banked T-cell subpopulation having the highest activity against each targeted TAA through one or more HLA-alleles shared between the patient and the T-cell subpopulations, wherein each T-cell subpopulation is specific for a single tumor associated antigen, wherein each of the T-cell subpopulations is specific for a different tumor associated antigen, wherein each of the T-cell subpopulations are primed and expanded separately from each other, wherein each of the T-cell subpopulations are primed and expanded ex vivo;
v) combining each selected banked T-cell subpopulation to create a MUSTANG composition; and,
vi) administering an effective amount of the MUSTANG composition to the patient.

In some embodiments, the shared HLA alleles are selected from one or more of HLA-A, HLA-B, or HLA-DR.

In some embodiments, the T-cell subpopulations used to create the MUSTANG composition are combined in a ratio or percentage that correlates with the relative identified TAA expression profile of the patient. In some embodiments, the ratio or percentage of each T-cell subpopulation is normalized based on the measured activity of each T-cell subpopulation against the TAA, for example, but not limited to, as measured by the EliSpot assay. In some embodiments, the TAA to target by the T-cell subpopulations used to create the MUSTANG composition are selected by the healthcare practitioner based on the type of tumor that is diagnosed. In some embodiments, the T-cell subpopulations used to create the MUSTANG composition are combined in about an equal ratio. In some embodiments, the T-cell subpopulations used to create the MUSTANG composition are combined in a variable ratio. In some embodiments, the MUSTANG composition comprises a first T-cell subpopulation and a second T-cell subpopulation, wherein the first T-cell subpopulation is specific for a different TAA than the second T-cell subpopulation. In some embodiments, the ratio of the first and second T-cell subpopulations is fixed at an equal ratio of 1:1, wherein the ratio is based on either total cell number or normalized cell activity. In an alternative embodiment, the separate T-cell subpopulations are not combined into a single dosage form, but rather administered as separate compositions, wherein the separate compositions are administered concomitantly in a ratio described above.

The ratios of the T-cell subpopulations in the MUSTANG composition may be selected based on the knowledge of the patient's tumor characteristics or the health provider's best judgement. In some embodiments, the composition comprises (i) at least about 45%, 50%, 55%, 60%, 65%, 70%, 75%, 80%, or 85% of a first T-cell subpopulation and (ii) at least about 15%, 20%, 25%, 30%, 35%, 40%, 45%, 50%, or 55% of a second T-cell subpopulation, wherein the percentage adds to 100% by weight. In some embodiments, the ratio or percentage of each T-cell subpopulation is normalized based on the measured activity of each T-cell subpopulation against the TAA as measured by, for example, but not limited to, the EliSpot assay. In some embodiments, the percentage of the first and second T-cell subpopulations is based on the TAA expression profile of a malignancy or tumor such that the percentage of the first and second T-cell subpopulations correlates with the TAA expression profile of the tumor.

The MUSTANG composition can include two, three, four, five, or more T-cell subpopulations. The T-cell subpopulations can be included in the MUSTANG composition in about an equal ratio, or in a ratio that reflects the individual TAA expression as determined by the patient's TAA expression profile. In an alternative embodiment, the T-cell subpopulations can be included in a ratio that reflects a greater percentage of T-cell subpopulations directed to known TAAs which show high immunogenicity. In some embodiments, the ratio or percentage of each T-cell subpopulation is normalized based on the measured activity of each T-cell subpopulation against the TAA as measured by the Eli Spot assay.

In a typical embodiment, a patient, such as a human, is infused or injected with an effective dose of a MUSTANG composition ranging from 1×10⁶ to 1×10⁸ cells/m². Alternatively, the T-cell subpopulations of a MUSTANG composition are not combined into a single dosage form, but rather each T-cell subpopulation is administered separately. The patient may receive a second or additional infusion or injection about 1 or more weeks later if recommended by the health care practitioner and may receive additional doses subsequent thereto as useful and recommended.

In one aspect, provided herein is a method of treating a patient with a tumor comprising:
i) determining the HLA subtype of the patient;
ii) determining the TAA expression profile of the patient's tumor;
iii) identifying two or more tumor associated antigens expressed by the patient's tumor for targeting with TAA-specific T-cell subpopulations;
iv) selecting one banked T-cell subpopulation having the highest activity against each targeted TAA through one or more HLA-alleles shared between the patient and the T-cell subpopulations, wherein each T-cell subpopulation is specific for a single tumor associated antigen, wherein each of the T-cell subpopulations is specific for a different tumor associated antigen, wherein each of the T-cell subpopulations are primed and expanded separately from each other, wherein each of the T-cell subpopulations are primed and expanded ex vivo;
v) combining each selected banked T-cell subpopulation to create a first MUSTANG composition; and,
vi) administering an effective amount of the first MUSTANG composition to the patient,
vii) monitoring the patient's response to the first MUSTANG composition by measuring the presence of circulating TAA specific T-cells;
viii) monitoring changes to the patient's TAA expression profile;
ix) if the patient's TAA expression profile has changed, identifying two or more tumor associated antigens expressed by the patient's tumor for targeting with TAA-specific T-cell subpopulations, wherein if the patient is showing a robust response to any specific TAA T-cell subpopulation(s) from the first MUSTANG composition, exclude targeting that TAA;
x) selecting one banked T-cell subpopulation having the highest activity against each targeted TAA from step ix) through one or more HLA-alleles shared between the patient and the T-cell subpopulations, wherein each T-cell subpopulation is specific for a single tumor associated antigen, wherein each of the T-cell subpopulations is specific for a different tumor associated antigen, wherein each of the T-cell subpopulations are primed and expanded separately from each other, wherein each of the T-cell subpopulations are primed and expanded ex vivo;
xi) combining each selected banked T-cell subpopulation to create a second MUSTANG composition;
xii) administering an effective amount of the second MUSTANG composition to the patient;
xiii) optionally repeating steps viii) to xii); and,
xiv) combining each selected banked T-cell subpopulation to create a third MUSTANG composition; and,
xv) administering an effective amount of the third MUSTANG composition to the patient.

In some embodiments, the shared HLA alleles are selected from one or more of HLA-A, HLA-B, or HLA-DR.

In an alternative embodiment, the separate T-cell subpopulations are not combined into a single dosage form, but rather administered as separate compositions, wherein the separate compositions are administered concomitantly.

By monitoring the levels of circulating TAA-specific T-cells and the TAA expression profile of the patient's tumor following administration of the first MUSTANG composition, the T-cell subpopulations included in any second, third, or subsequently administered MUSTANG composition can be adjusted, providing a more tailored approach to treatment as a tumor progresses. For example, if after an initial administration of a MUSTANG composition containing for example a T-cell subpopulation to PRAME, if high levels of circulating PRAME-specific T-cells are measured, then it may not be necessary to include a PRAME-specific T-cell subpopulation in the subsequently administered MUSTANG compositions. Similarly, if after an initial administration of a MUSTANG composition containing for example a PRAME T-cell subpopulation, a significant reduction in PRAME expression is measured by TAA expression profile of the patient's tumor, then it may not be necessary to include the PRAME-specific T-cell subpopulation in subsequent MUSTANG compositions. Accordingly, the subsequently administered MUSTANG compositions may be modified to more closely reflect the tumor associated antigen expression profile of the tumor. In addition, the subsequently administered MUSTANG compositions may be modified based on the ongoing T-cell subpopulation responses in vivo, whereby previously administered T-cell subpopulations showing robust activity in vivo are not included in subsequent MUSTANG compositions because additional administrations of that specific T-cell subpopulation may be unnecessary. In some embodiments, the first, second, and any subsequent MUSTANG compositions are comprised of T-cell subpopulations derived from the same donor. In an alternative embodiment, the first, second, and subsequent MUSTANG compositions may be derived from different donors, provided that one of the donors is a non-cord blood donor.

In one aspect, provided herein is a method of treating a patient with a tumor comprising:
i) determining the HLA subtype of the patient;
ii) diagnosing the tumor type of the patient;
iii) identifying a pre-selected set of two or more tumor associated antigens associated with the tumor type for targeting with TAA-specific T-cell subpopulations;
iv) selecting one banked T-cell subpopulation having the highest activity against each targeted TAA through one or more HLA-alleles shared between the patient and the T-cell subpopulations, wherein each T-cell subpopulation is specific for a single tumor associated antigen, wherein each of the T-cell subpopulations is specific for a different tumor associated antigen, wherein each of the T-cell subpopulations are primed and expanded separately from each other, wherein each of the T-cell subpopulations are primed and expanded ex vivo;
v) combining each selected banked T-cell subpopulation to create a first MUSTANG composition;
vi) administering an effective amount of the first MUSTANG composition to the patient.
vii) monitoring the patient's response to the first MUSTANG composition by measuring the presence of circulating TAA specific T-cells;
viii) monitoring changes to the patient's TAA expression profile;
ix) if the patient's TAA expression profile has changed, identifying two or more tumor associated antigens expressed by the patient's tumor for targeting with TAA-specific T-cell subpopulations, wherein if the patient is showing a robust response to any specific TAA T-cell subpopulation(s) from the first MUSTANG composition, exclude targeting that TAA;
x) selecting one banked T-cell subpopulation having the highest activity against each targeted TAA from step ix) through one or more HLA-alleles shared between the patient and the T-cell subpopulations, wherein each T-cell subpopulation is specific for a single tumor associated antigen, wherein each of the T-cell subpopulations is specific for a different tumor associated antigen, wherein each of the T-cell subpopulations are primed and expanded separately from each other, wherein each of the T-cell subpopulations are primed and expanded ex vivo;
xi) combining each selected banked T-cell subpopulation to create a second MUSTANG composition;
xii) administering an effective amount of the second MUSTANG composition to the patient;
xiii) optionally repeating steps viii) to xii); and,
xiv) combining each selected banked T-cell subpopulation to create a third MUSTANG composition; and,
xv) administering an effective amount of the third MUSTANG composition to the patient.

In some embodiments, instead of using a banked T cell subpopulation, a newly produced T cell subpopulation, that has yet to be banked, can be used. In some aspects, a portion of the newly produced T cell subpopulation can be used to treat a patient and another portion can be banked for future use. In some embodiments, the shared HLA alleles are selected from one or more of HLA-A, HLA-B, or HLA-DR.

In an alternative embodiment, the separate T-cell subpopulations are not combined into a single dosage form, but rather administered as separate compositions, wherein the separate compositions are administered concomitantly. By initially administering to the patient a first MUSTANG composition comprising T-cell subpopulations targeting a pre-determined set of TAAs based on the type of tumor, immediate T-cell therapy can be initiated and the therapy further tailored by determining the patient's response to the first MUSTANG composition and TAA expression profile and adjusting the T-cell subpopulations of the second (and subsequent) MUSTANG compositions. The timing of determining the TAA expression profile of the patient's tumor—can be performed before or after the administration of the first MUSTANG composition. In some embodiments, the pre-determined TAAs targeted by the T-cell subpopulations of the first MUSTANG composition are selected from WT1, PRAME, Survivin, NY-ESO-1, MAGE-A3, MAGE-A4, Pr3, Cyclin A1, SSX2, Neutrophil Elastase (NE), HPV E6. HPV E7, EBV EBV LMP2, EBV EBNA1, and EBV EBNA2, or any combination thereof. In some embodiments, the first MUSTANG composition is comprised of T-cell subpopulations that separately target one of PRAME, WT1, and survivin, respectively. In some embodiments, the first MUSTANG composition is comprised of T-cell subpopulations that separately target PRAME, WT1, and survivin. Furthermore, additional MUSTANG composition administrations, for example a fourth, fifth, sixth, seventh, or more, can occur by following the protocol outlined above.

The T-cells can be primed and activated using a number of known procedures. In one non-limiting aspect, the present invention includes a process for generating a T-cell subpopulation specific to a single TAA to form MUSTANG therapeutic compositions that includes but is not limited to:
i) identifying eligible donors who are negative to the patient's disease, and preferably healthy, and wherein the donor sample can be cord blood or PBMCs;
ii) collecting the mononuclear cells from the healthy donor and optionally removing any effector or other memory T-cells optionally based on CD45RA⁻, CD45RO⁺, CCR7⁻, CD62L⁻, CCR7⁺, and/or CD62L⁺ markers;
iii) separating the mononuclear cells into two components;
iv) separating the cells in the first component into nonadherent T-cells and precursors and adherent dendritic cells and precursors, using any method known in the art, for example exposure to a solid medium, separation magnetically, use of antibodies, etc., and if not done already, optionally removing any effector or other memory T-cells optionally based on CD45RA−, CD45RO+, CCR7, CD62L−, or CCR7+, CD62L+ markers;
v) differentiating monocytes and precursors to dendritic cells with IL-4 and GM-CSF, followed by treatment with maturing cytokines such as LPS, TNFα, IL-1β, IL-4, IL-6 and GM-CSF and then pulsing with one or more peptide(s) and/or epitope(s) from a single selected TAA; and then irradiating to form dendritic antigen presenting cells (APCs); vi) treating the nonadherent T-cells and precursors with cytokines IL-7 and IL-15 to polarize to Th1 cells (and in some embodiments, without the use of IL-12);
vii) mixing the dendritic antigen presenting cells from (v) with the non-adherent T-cells and T-cell precursor cells from (vi) in the presence of cytokines IL-6, optionally in a ratio of between 5:1 and 20:1 of (vi) to (v) to produce a T-cell subpopulation specific for a single TAA;
viii) treating the second component of mononuclear cells with a mitogen such as PHA, a T-blast, B-blast, lymphoblastic cell or CD3/CD28 Blast optionally in the presence of IL-2 to produce activated T-cells; and then irradiating the cells to inhibit growth; ix) pulsing the PHA blasts in (viii) with selected antigenic peptide(s) and/or epitope(s) from the single selected tumor-associated antigen and irradiating to inhibit growth;
x) mixing the antigen specific T-cells from (vii) with the activated T-cell subpopulation from (ix) optionally in the presence of K562 accessory cells (preferably HLA-negative, K562 cells expressing CD80, CD83, CD86 and/or 4-IBBL) or LPS, and optionally IL-15 and/or IL-2;
xi) recovering the produced single TAA-specific T-cell subpopulation;
xii) optionally characterizing the resulting T-cell subpopulation for banking; and,
xiii) optionally cryopreserving and storing in the bank until use.

In some embodiments, the TAA peptides used to prime and expand a T-cell subpopulation in step (v) are from a library of overlapping peptide fragments of the tumor antigen, as provided for example, in commercially available overlapping peptide libraries. In some embodiments, the TAA peptides used to prime and expand a T-cell subpopulation in step (v) are from a library of overlapping peptide fragments of the tumor antigen, as provided for example, in commercially available overlapping peptide libraries, wherein the library been further enriched with one or more specific known or identified epitopes expressed by the patient's tumor. In some embodiments, the TAA peptides used to prime and expand a T-cell subpopulation in step (v) include specifically selected HLA-restricted peptides generated by determining the HLA profile of the donor source, and including peptide epitopes derived from the targeted TAA that are active through the donor's HLA type.

In the above process, unless specific steps are taken to remove cell components of the donor blood starting material, for example, removal based on cell surface markers, etc., the final T-cell subpopulation will normally also include a range of cell types, such as Natural Killer T-cells, γδ T-cells, CD4+ T-cells, CD8+(cytotoxic) T-cells, and Natural Killer T-cells, among others, and may have naïve, and effector memory or central memory cells. The ratios of these cell types in the MUSTANG composition will vary according to the donor's blood and processing conditions. In another aspect, the present invention includes a method of manufacturing a T-cell subpopulation of the present invention comprising (i) collecting a mononuclear cell product from a healthy donor; (ii) determining the HLA subtype of the mononuclear cell product; (iii) separating the monocytes and the lymphocytes of the mononuclear cell product; (iv) generating and maturing dendritic cells (DCs) from the monocyte fraction; (v) pulsing the DCs with one or more peptides and/or epitopes from a single TAA; (vi) carrying out a CD45RA+ selection to isolate naïve lymphocytes from the lymphocyte fraction; (vii) stimulating the naïve lymphocytes with the peptide-pulsed DCs in the presence of a cytokine cocktail; (viii) repeating the T cell stimulation with fresh peptide-pulsed DCs or other peptide-pulsed antigen presenting cells in the presence of a cytokine cocktail; and (ix) harvesting the T-cell subpopulation, (x) characterizing the T-cell subpopulation as described herein; and (xi) banking the T-cell subpopulation for future use in a MUSTANG composition. In some embodiments, the TAA peptides used to prime and expand a T-cell subpopulation in step (v) are from a library of overlapping peptide fragments of the tumor antigen, as provided for example, in commercially available overlapping peptide libraries. In some embodiments, the TAA peptides used to prime and expand a T-cell subpopulation in step (v) are from a library of overlapping peptide fragments of the tumor antigen, as provided for example, in commercially available overlapping peptide libraries, wherein the library been further enriched with one or more specific known or identified epitopes expressed by the patient's tumor. In some embodiments, the TAA peptides used to prime and expand a T-cell subpopulation in step (v) include specifically selected HLA-restricted peptides generated by determining the HLA profile of the donor source, and including peptide epitopes derived from the targeted TAA that are active through the donor's HLA type. In another aspect, the present invention includes a method of manufacturing a T-cell subpopulation of the present invention comprising (i) collecting a mononuclear cell product from a healthy donor; (ii) determining the HLA subtype of the mononuclear cell product; (iii) separating the monocytes and the lymphocytes of the mononuclear cell product; (iv) generating and maturing dendritic cells (DCs) from the monocyte fraction; (v) pulsing the DCs with one or more peptides and/or epitopes from a single TAA; (vi) carrying out a CD45RA+ selection to isolate naïve T cells from the lymphocyte fraction; (vii) stimulating the naïve T cells with the peptide-pulsed DCs in the presence of a cytokine cocktail; (viii) repeating the T cell stimulation with fresh peptide-pulsed DCs or other peptide-pulsed antigen presenting cells in the presence of a cytokine cocktail creating a primed T-cell subpopulation; (ix) harvesting the primed T-cell subpopulation, (x) characterizing the primed T-cell subpopulation as described herein; and (xi) banking the primed T-cell subpopulation for future use in a MUSTANG composition.

In a further aspect, the present invention includes a bank of isolated T-cell subpopulations targeting a TAA comprising two or more characterized T-cell subpopulations. The T-cell subpopulations are characterized, the characterization is recorded in a database for future use, and the T-cell subpopulations cryopreserved. The T-cell subpopulation has been characterized by, for example, HLA-phenotype, its specificity to its specific TAA, the epitope or epitopes each T-cell subpopulation is specific to, which MHC Class I and Class II the T-cell subpopulation is restricted to, antigenic activity through the T-cell's corresponding HLA-allele, and immune effector subtype concentration.

### BRIEF DESCRIPTION OF FIGURES

FIG. 1: Schematic of the generation of antigen-specific T-cell lines. Healthy donor PBMC were primed with autologous dendritic cells pulsed with 3 TAAs (Survivin, WT1, and PRAME) at an effector-to-target ratio of 10:1 in the presence of a cytokine-mix containing IL7, IL12, IL15, and IL6. For the subsequent stimulations IL2 and IL7 was used. For the further maintenance of the T-cells, IL15 and IL2 was used after the first stimulation.

FIG. 2: IFN-γ-ELISpot assay correlating with the recognition of specific tumor associated antigens by T-cells generated using a multi-TAA PepMix™. The x-axis contains the TAA mix (positive control), the specific antigens separately (PRAME, WT1, and Survivin), and negative controls. The y-axis is mean spot count as measured by SFU/2×10⁵ cells.

FIG. 3: IFN-γ-ELISpot assay correlating with the recognition of specific tumor associated antigens. The x-axis represents each of 21 T-cell subpopulations generated by using a multi-TAA PepMix™. The y-axis is mean spot count as measured by IFNγ SFC/2×10⁵ cells, which serves as a measure of the specificity of each generated T-cell subpopulation to the specific antigens (PRAME, WT1, and Survivin).

FIG. 4: Pie charts depicting the number of antigens recognized in IFNγ-ELISpot by T-cell populations generated against i) multi-TAA PepMixes™ (left) and 2) individual TAAs in separate cultures (right). See Weber et al., Generation of tumor multi-leukemia antigen-specific T cells to enhance the graft-versus-leukemia effect after allogeneic stem cell transplant, Leukemia 2013; 27, 1538-1547 (FIG. 1d).

FIG. 5: IFN-γ-ELISpot assay correlating with the recognition of specific tumor associated antigens by T-cells generated using a multi-TAA PepMix™. The x-axis contains the TAA mix (positive control), the specific antigens of the TAA mix separately (WT1, NE, Pr3, MAGE, and PRAME), and negative controls. The y-axis is mean spot count as measured by SFU/2×10⁵ cells. See Weber et al., Generation of tumor multi-leukemia antigen-specific T cells to enhance the graft-versus-leukemia effect after allogeneic stem cell transplant, Leukemia 2013; 27, 1538-1547 (FIG. 1e).

FIG. 6: Mean cytolytic activity±s.e. of T-cell populations (n=8) against peptide-pulsed autologous PHA-blasts (PHAB) in a carboxyfluorescein succinimidyl ester (CFSE)-based cytotoxicity assay at an effector-to-target ratio of 40:1. The T-cell populations were generated using a multi-TAA PepMix™. The x-axis contains the TAA mix (positive control), the specific antigens of the TAA mix separately (WT1, NE, Pr3, MAGE, and PRAME), and negative controls. The y-axis is percent lysis of PHA-blasts. See Weber et al., Generation of tumor multi-leukemia antigen-specific T cells to enhance the graft-versus-leukemia effect after allogeneic stem cell transplant, Leukemia 2013; 27, 1538-1547 (FIG. 1f).

FIG. 7: Exemplary cytotoxic assay. T-cell populations generated using a multi-TAA PepMix™ were co-cultured with partially HLA-matched AML blast sample. Leukemia blasts were quantified by anti-CD33/CD34 co-staining and T-cells by CD3 staining. Leukemia blasts were eliminated over time when co-cultured with T-cells generated with a multi-TAA PepMix™ but remained at higher levels in culture when incubated with control T-cells from the same donor. Analysis on day 0 and after 1 and 3 days of co-culture.

FIG. 8: Schematic of the generation of antigen-specific T-cell lines using a single tumor antigen peptide or peptide library. Healthy donor PBMC are primed with autologous dendritic cells pulsed with a single TAA at an effector-to-target ratio of 10:1 in the presence of a cytokine-mix containing IL7, IL12, IL15, and IL6. For the subsequent stimulations IL2 and IL7 is used. For the further maintenance of CTLs IL15 and IL2 is used after the first stimulation.

FIG. 9: Exemplary schematic showing that from a single donor a T-cell subpopulation can be generated that could be used for multiple patients who share HLA alleles that have TAA activity. In this example, a T-cell subpopulation expanded from this donor has TAA activity through the HLA-I B8 and HLA-II DR1. Thus, these T-cell subpopulations could be used for any of these 3 patients since at least one shared HLA alleles has TAA activity.

FIG. 10: IFN-γ-ELISpot assay correlating with the recognition of specific tumor associated antigens by T-cells generated using a multi-TAA overlapping peptide library (WT1, PRAME, and Survivin) and individual overlapping peptide libraries to each TAA. The x-axis contains the TAA mix (WT1, PRAME, and Survivin) and the specific antigens separately (WT1, PRAME, and Survivin) as well as a 1:1:1 cell-to-cell ratio of the single antigen T-cell populations. The y-axis is mean spot count as measured by the log SFU per 10⁵ cells normalized to actin (positive control).

### DETAILED DESCRIPTION OF THE INVENTION

Provided herein are improved adoptive T-cell therapies to treat human tumors which include administering to a patient in need thereof an effective amount of a T-cell composition that includes in the same dosage form a multiplicity of T-cell subpopulations, wherein each T-cell subpopulation is specific for a single tumor-associated antigen (TAA), and the T-cell subpopulations that comprise the T-cell composition for administration are chosen specifically based on the TAA expression profile of the patient's tumor.

Further, importantly, this advantageous T-cell therapy can be optimized for personal efficacy in the host by testing each T-cell subpopulation separately for potential responsiveness in vivo against the patient's tumor.

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “allogeneic” as used herein refers to medical therapy in which the donor and recipient are different individuals of the same species.

The term “antigen” as used herein refers to molecules, such as polypeptides, peptides, or glyco- or lipo-peptides that are recognized by the immune system, such as by the cellular or humoral arms of the human immune system. The term “antigen” includes antigenic determinants, such as peptides with lengths of 6, 7, 8, 9, 10, 11, 12, 13, 14, 15, 16, 17, 18, 19, 20, 21, 22 or more amino acid residues that bind to MHC molecules, form parts of MHC Class I or II complexes, or that are recognized when complexed with such molecules.

The term “antigen presenting cell (APC)” as used herein refers to a class of cells capable of presenting one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen.

The term “autologous” as used herein refers to medical therapy in which the donor and recipient are the same person.

The term “cord blood” as used herein has its normal meaning in the art and refers to blood that remains in the placenta and umbilical cord after birth and contains hematopoietic stem cells. Cord blood may be fresh, cryopreserved, or obtained from a cord blood bank.

The term “cytokine” as used herein has its normal meaning in the art. Nonlimiting examples of cytokines used in the invention include IL-2, IL-6, IL-7, IL-12, IL-15, and IL-27.

The term “cytotoxic T-cell” or “cytotoxic T lymphocyte” as used herein is a type of immune cell that bears a CD8+ antigen and that can kill certain cells, including foreign cells, tumor cells, and cells infected with a virus. Cytotoxic T cells can be separated from other blood cells, grown ex vivo, and then given to a patient to kill tumor or viral cells. A cytotoxic T cell is a type of white blood cell and a type of lymphocyte.

The term “dendritic cell” or “DC” as used herein describes a diverse population of morphologically similar cell types found in a variety of lymphoid and non-lymphoid tissues, see Steinman, Ann. Rev. Immunol. 9:271-296 (1991).

The term “effector cell” as used herein describes a cell that can bind to or otherwise recognize an antigen and mediate an immune response. Tumor, virus, or other antigen-specific T-cells and NKT-cells are examples of effector cells.

The term “endogenous” as used herein refers to any material from or produced inside an organism, cell, tissue or system.

The term “epitope” or “antigenic determinant” as used herein refers to the part of an antigen that is recognized by the immune system, specifically by antibodies, B cells, or T cells.

The term “exogenous” as used herein refers to any material introduced from or produced outside an organism, cell, tissue or system.

The term “HLA” as used herein refers to human leukocyte antigen. There are 7,196 HLA alleles. These are divided into 6 HLA class I and 6 HLA class II alleles for each individual (on two chromosomes). The HLA system or complex is a gene complex encoding the major histocompatibility complex (MHC) proteins in humans. HLAs corresponding to MHC Class I (A, B, or C) present peptides from within the cell and activate CD8-positive (i.e., cytotoxic) T-cells. HLAs corresponding to MHC Class II (DP, DM, DOA, DOB, DQ and DR) stimulate the multiplication of CD4-positive T-cells) which stimulate antibody-producing B-cells.

The term “isolated” as used herein means separated from components in which a material is ordinarily associated with, for example, an isolated cord blood mononuclear cell can be separated from red blood cells, plasma, and other components of cord blood.

A “naive” T-cell or other immune effector cell as used herein is one that has not been exposed to or primed by an antigen or to an antigen-presenting cell presenting a peptide antigen capable of activating that cell.

The term “non-engineered” when referring to the cells of the compositions means a cell that does not contain or express an exogenous nucleic acid or amino acid sequence. For example, the cells of the compositions do not express, for example, a chimeric antigen receptor.

A “peptide library” or “overlapping peptide library” as used herein within the meaning of the application is a complex mixture of peptides which in the aggregate covers the partial or complete sequence of a protein antigen. Successive peptides within the mixture overlap each other, for example, a peptide library may be constituted of peptides 15 amino acids in length which overlapping adjacent peptides in the library by 11 amino acid residues and which span the entire length of a protein antigen. Peptide libraries may be commercially available or may be custom-made for particular antigens.

A “peripheral blood mononuclear cell” or “PBMC” as used herein is any peripheral blood cell having a round nucleus. These cells consist of lymphocytes (T cells, B cells, NK cells) and monocytes. In humans, lymphocytes make up the majority of the PBMC population, followed by monocytes, and a small percentage of dendritic cells.

The term “precursor cell” as used herein refers to a cell which can differentiate or otherwise be transformed into a particular kind of cell. For example, a “T-cell precursor cell” can differentiate into a T-cell and a “dendritic precursor cell” can differentiate into a dendritic cell.

A “subject” or “host” or “patient” as used herein is a vertebrate, preferably a mammal, more preferably a human. Mammals include, but are not limited to humans, simians, equines, bovines, porcines, canines, felines, murines, other farm animals, sport animals, or pets. Humans include those in need of virus- or other antigen-specific T-cells, such as those with lymphocytopenia, those who have undergone immune system ablation, those undergoing transplantation and/or immunosuppressive regimens, those having naïve or developing immune systems, such as neonates, or those undergoing cord blood or stem cell transplantation. In a typical embodiment, the term “patient” as used herein refers to a human.

A “T-cell population” or “T-cell subpopulation” can include thymocytes, immature T lymphocytes, mature T lymphocytes, resting T lymphocytes and activated T-lymphocytes. The T-cell population or subpopulation can include αβ T-cells, including CD4+ T-cells, CD8+ T cells, γδ T-cells, Natural Killer T-cells, or any other subset of T-cells.

The term “tumor-associated antigen expression profile” or “tumor antigen expression profile” as used herein, refers to a profile of expression levels of tumor-associated antigens within a malignancy or tumor. Tumor-associated antigen expression may be assessed by any suitable method known in the art including, without limitation, quantitative real time polymerase chain reaction (qPCR), cell staining, or other suitable techniques. Non-limiting exemplary methods for determining a tumor-associated antigen expression profile can be found in Ding et al., Cancer Bio Med (2012) 9: 73-76; Qin et al., Leukemia Research (2009) 33(3) 384-390; Weber et al., Leukemia (2009) 23: 1634-1642; Liu et al., J. Immunol (2006) 176: 3374-3382; Schuster et al., Int J Cancer (2004) 108: 219-227.

The term “tumor-associated antigen” or “TAA” as used herein is an antigen that is highly correlated with certain tumor cells. They are not usually found, or are found to a lesser extent, on normal cells.

The term “MUSTANG composition” refers to a “MUltiple Single Tumor ANtiGen” T-cell composition” composition. The MUSTANG composition is comprised of two or more T-cell subpopulations, wherein each T-cell subpopulation targets a single tumor-associated antigen. For purposes herein, when referring to combining T-cell subpopulations to comprise the MUSTANG composition, combining is intended to include the situation wherein the T-cells are physically combined into a single dosage form, that is, a single composition. In alternative embodiments, the T-cells subpopulations are kept physically separated but administrated concomitantly and collectively comprise the MUSTANG composition.

### Tumor-Associated Antigens

The careful selection of antigens for MUSTANG composition therapy is critical to success. Antigens used for immunotherapy should be intentionally selected based on either uniqueness to tumor cells, greater expression in tumor cells as compared to normal cells, or ability of normal cells with antigen expression to be adversely affected without significant compromise to normal cells or tissue.

Tumor-associated antigens (TAA) can be loosely categorized as oncofetal (typically only expressed in fetal tissues and in cancerous somatic cells), oncoviral (encoded by tumorigenic transforming viruses), overexpressed/accumulated (expressed by both normal and neoplastic tissue, with the level of expression highly elevated in neoplasia), cancer-testis (expressed only by cancer cells and adult reproductive tissues such as testis and placenta), lineage-restricted (expressed largely by a single cancer histotype), mutated (only expressed by cancer as a result of genetic mutation or alteration in transcription), post-translationally altered (tumor-associated alterations in glycosylation, etc.), or idiotypic (highly polymorphic genes where a tumor cell expresses a specific “clonotype”, i.e., as in B cell, T cell lymphoma/leukemia resulting from clonal aberrancies). Although they are preferentially expressed by tumor cells, TAAs are oftentimes found in normal tissues. However, their expression differs from that of normal tissues by their degree of expression in the tumor, alterations in their protein structure in comparison with their normal counterparts or by their aberrant subcellular localization within malignant or tumor cells.

Examples of oncofetal tumor associated antigens include Carcinoembryonic antigen (CEA), immature laminin receptor, and tumor-associated glycoprotein (TAG) 72. Examples of overexpressed/accumulated include BING-4, calcium-activated chloride channel (CLCA) 2, Cyclin A₁, Cyclin B₁, 9D7, epithelial cell adhesion molecule (Ep-Cam), EphA3, Her2/neu, telomerase, mesothelin, orphan tyrosine kinase receptor (ROR1), stomach cancer-associated protein tyrosine phosphatase 1 (SAP-1), and survivin.

Examples of cancer-testis antigens include the b melanoma antigen (BAGE) family, cancer-associated gene (CAGE) family, G antigen (GAGE) family, melanoma antigen (MAGE) family, sarcoma antigen (SAGE) family and X antigen (XAGE) family, CT9, CT10, NY-ESO-1, L antigen (LAGE) 1, Melanoma antigen preferentially expressed in tumors (PRAME), and synovial sarcoma X (SSX) 2. Examples of lineage restricted tumor antigens include melanoma antigen recognized by T cells-1/2 (Melan-A/MART-1/2), Gp100/pmel17, tyrosine-related protein (TRP) 1 and 2, P. polypeptide, melanocortin 1 receptor (MC1R), and prostate-specific antigen. Examples of mutated tumor antigens include β-catenin, breast cancer antigen (BRCA) 1/2, cyclin-dependent kinase (CDK) 4, chronic myelogenous leukemia antigen (CML) 66, fibronectin, p53, Ras, and TGF-βRII. An example of a post-translationally altered tumor antigen is mucin (MUC) 1. Examples of idiotypic tumor antigens include immunoglobulin (Ig) and T cell receptor (TCR).

In some embodiments, the antigen associated with the disease or disorder is selected from the group consisting of CD19, CD20, CD22, hepatitis B surface antigen, anti-folate receptor, CD23, CD24, CD30, CD33, CD38, CD44, EGFR, EGP-2, EGP-4, OEPHa2, ErbB2, 3, or 4, FBP, fetal acetylcholine receptor, HMW-MAA, IL-22R-alpha, IL-13R-alpha, kdr, kappa light chain, Lewis Y, MUC16 (CA-125), PSCA, NKG2D Ligands, oncofetal antigen, VEGF-R2, PSMA, estrogen receptor, progesterone receptor, ephrinB2, CD123, CS-1, c-Met and/or biotinylated molecules, and/or molecules expressed by HIV, HCV, HBV or other pathogens.

Exemplary tumor antigens include at least the following: carcinoembryonic antigen (CEA) for bowel cancers; CA-125 for ovarian cancer; MUC1 or epithelial tumor antigen (ETA) or CA15-3 for breast cancer; tyrosinase or melanoma-associated antigen (MAGE) for malignant melanoma; and abnormal products of ras, p53 for a variety of types of tumors; alphafetoprotein for hepatoma, ovarian, or testicular cancer; beta subunit of hCG for men with testicular cancer; prostate specific antigen for prostate cancer; beta 2 microglobulin for multiple myeloma and in some lymphomas; CA19-9 for colorectal, bile duct, and pancreatic cancer; chromogranin A for lung and prostate cancer; TA90 for melanoma, soft tissue sarcomas, and breast, colon, and lung cancer. Examples of TAAs are known in the art, for example in N. Vigneron, “Human Tumor Antigens and Cancer Immunotherapy,” BioMed Research International, vol. 2015, Article ID 948501, 17 pages, 2015. doi:10.1155/2015/948501; Ilyas et al., J Immunol. (2015) Dec. 1; 195(11): 5117-5122; Coulie et al., Nature Reviews Cancer (2014) volume 14, pages 135-146; Cheever et al., Clin Cancer Res. (2009) Sep. 1; 15(17):5323-37, which are incorporated by reference herein in its entirety.

Examples of oncoviral TAAs include human papilloma virus (HPV) L1, E6 and E7, Epstein-Barr Virus (EBV) Epsteing-Barr nuclear antigen (EBNA) 1 and 2, EBV viral capsid antigen (VCA) Igm or IgG, EBV early antigen (EA), latent membrane protein (LMP) 1 and 2, hepatitis B surface antigen (HBsAg), hepatitis B e antigen (HBeAg), hepatitis B core antigen (HBcAg), hepatitis B x antigen (HBxAg), hepatitis C core antigen (HCV core Ag), Human T-Lymphotropic Virus Type 1 core antigen (HTLV-1 core antigen), HTLV-1 Tax antigen, HTLV-1 Group specific (Gag) antigens, HTLV-1 envelope (Env), HTLV-1 protease antigens (Pro), HTLV-1 Tof, HTLV-1 Rof, HTLV-1 polymerase (Pro) antigen, Human T-Lymphotropic Virus Type 2 core antigen (HTLV-2 core antigen), HTLV-2 Tax antigen, HTLV-2 Group specific (Gag) antigens, HTLV-2 envelope (Env), HTLV-2 protease antigens (Pro), HTLV-2 Tof, HTLV-2 Rof, HTLV-2 polymerase (Pro) antigen, latency-associated nuclear antigen (LANA), human herpesvirus-8 (HHV-8) K8.1, Merkel cell polyomavirus large T antigen (LTAg), and Merkel cell polyomavirus small T antigen (sTAg).

Elevated expression of certain types of glycolipids, for example gangliosides, is associated with the promotion of tumor survival in certain types of cancers. Examples of gangliosides include, for example, GM1b, GD1c, GM3, GM2, GM1a, GD1a, GT1a, GD3, GD2, GD1b, GT1b, GQ1b, GT3, GT2, GT1c, GQ1c, and GP1c. Examples of ganglioside derivatives include, for example, 9-O-Ac-GD3, 9-O-Ac-GD2, 5-N-de-GM3, N-glycolyl GM3, NeuGcGM3, and fucosyl-GM1. Exemplary gangliosides that are often present in higher levels in tumors, for example melanoma, small-cell lung cancer, sarcoma, and neuroblastoma, include GD3, GM2, and GD2.

In addition to the TAAs described above, another class of TAAs is tumor-specific neoantigens, which arise via mutations that alter amino acid coding sequences (non-synonymous somatic mutations). Some of these mutated peptides can be expressed, processed and presented on the cell surface, and subsequently recognized by T cells. Because normal tissues do not possess these somatic mutations, neoantigen-specific T cells are not subject to central and peripheral tolerance, and also lack the ability to induce normal tissue destruction. See, e.g., Lu & Robins, Cancer Immunotherapy Targeting Neoantigens, Seminars in Immunology, Volume 28, Issue 1, February 2016, Pages 22-27, incorporated herein by reference.

In some embodiments, at least one T-cell subpopulation comprising the MUSTANG composition is specific to an oncofetal TAA selected from a group consisting of Carcinoembryonic antigen (CEA), immature laminin receptor, orphan tyrosine kinase receptor (ROR1), and tumor-associated glycoprotein (TAG) 72. In some embodiments, at least one T-cell subpopulation is specific to CEA. In some embodiments, at least one T-cell subpopulation is specific to immature laminin receptor. In some embodiments, at least one T-cell subpopulation is specific to ROR1. In some embodiments, at least one T-cell subpopulation is specific is specific to TAG72.

In some embodiments, a T-cell subpopulation comprising the MUSTANG composition is specific to an oncoviral TAA selected from a group consisting of human papilloma virus (HPV) E6 and E7, Epstein-Barr Virus (EBV) Epsteing-Barr nuclear antigen (EBNA) 1 and 2, latent membrane protein (LMP) 1, and LMP2. In some embodiments, at least one T-cell subpopulation is specific to HPV E6. In some embodiments, at least one T-cell subpopulation is specific to HPV E7. In some embodiments, at least one T-cell subpopulation is specific to EBV. In some embodiments, at least one T-cell subpopulation is specific to EBNA1. In some embodiments, at least one T-cell subpopulation is specific to EBNA2. In some embodiments, at least one T-cell subpopulation is specific to LMP1. In some embodiments, at least one T-cell subpopulation is specific to LMP2.

In some embodiments, a T-cell subpopulation comprising the MUSTANG composition is specific to an overexpressed/accumulated TAA selected from a group consisting of BING-4, calcium-activated chloride channel (CLCA) 2, CyclinA₁, Cyclin B₁, 9D7, epithelial cell adhesion molecule (Ep-Cam), EphA3, Her2/neu, L1 cell adhesion molecule (L1-Cam), telomerase, mesothelin, stomach cancer-associated protein tyrosine phosphatase 1 (SAP-1), and survivin. In some embodiments, at least one T-cell subpopulation is specific to BING-4. In some embodiments, at least one T-cell subpopulation is specific to CLCA2. In some embodiments, at least one T-cell subpopulation is specific to Cyclin A₁. In some embodiments, at least one T-cell subpopulation is specific to Cyclin B₁. In some embodiments, at least one T-cell subpopulation is specific to 9D7. In some embodiments, at least one T-cell subpopulation is specific Ep-Cam. In some embodiments, at least one T-cell subpopulation is specific to EphA3. In some embodiments, at least one T-cell subpopulation is specific to Her2/neu. In some embodiments, at least one T-cell subpopulation is specific to L1-Cam. In some embodiments, at least one T-cell subpopulation is specific to telomerase. In some embodiments, at least one T-cell subpopulation is specific to mesothelin. In some embodiments, at least one T-cell subpopulation is specific to SAP-1. In some embodiments, at least one T-cell subpopulation is specific to survivin.

In some embodiments, a T-cell subpopulation comprising the MUSTANG composition is specific to a cancer-testis antigen selected from the group consisting of the b melanoma antigen (BAGE) family, cancer-associated gene (CAGE) family, G antigen (GAGE) family, melanoma antigen (MAGE) family, sarcoma antigen (SAGE) family and X antigen (XAGE) family, cutaneous T cell lymphoma associated antigen family (cTAGE), Interleukin-13 receptor subunit alpha-1 (IL13RA), CT9, Putative tumor antigen NA88-A, leucine zipper protein 4 (LUZP4), NY-ESO-1, L antigen (LAGE) 1, helicase antigen (HAGE), lipase I (LIPI), Melanoma antigen preferentially expressed in tumors (PRAME), synovial sarcoma X (SSX) family, sperm protein associated with the nucleus on the chromosome X (SPANX) family, cancer/testis antigen 2 (CTAG2), calcium-binding tyrosine phosphorylation-regulated fibrous sheath protein (CABYR), acrosin binding protein (ACRBP), centrosomal protein 55 (CEP55) and Synaptonemal Complex Protein 1 (SYCP1). In some embodiments, at least one T-cell subpopulation is specific to the BAGE family. In some embodiments, at least one T-cell subpopulation is specific to the CAGE family. In some embodiments, at least one T-cell subpopulation is specific to the GAGE family. In some embodiments, at least one T-cell subpopulation is specific to the MAGE family. In some embodiments, at least one T-cell subpopulation is specific to the SAGE family. In some embodiments, at least one T-cell subpopulation is specific to the XAGE family. In some embodiments, at least one T-cell subpopulation is specific to the cTAGE family. In some embodiments, at least one T-cell subpopulation is specific to IL13RA. In some embodiments, at least one T-cell subpopulation is specific to CT9. In some embodiments, at least one T-cell subpopulation is specific to NA88-A. In some embodiments, at least one T-cell subpopulation is specific to LUZP4. In some embodiments, at least one T-cell subpopulation is specific to NY-ESO-1. In some embodiments, at least one T-cell subpopulation is specific to LAGE-1. In some embodiments, at least one T-cell subpopulation is specific to HAGE. In some embodiments, at least one T-cell subpopulation is specific to LIPI. In some embodiments, at least one T-cell subpopulation is specific to PRAME. In some embodiments, at least one T-cell subpopulation is specific to the SSX family. In some embodiments, at least one T-cell subpopulation is specific to the SPANX family. In some embodiments, at least one T-cell subpopulation is specific to CTAG2. In some embodiments, at least one T-cell subpopulation is specific to CABYR. In some embodiments, at least one T-cell subpopulation is specific to ACRBP. In some embodiments, at least one T-cell subpopulation is specific to CEP55. In some embodiments, at least one T-cell subpopulation is specific to SYCP1.

In some embodiments, a T-cell subpopulation comprising the MUSTANG composition is specific to a lineage restricted tumor antigen selected from the group consisting of melanoma antigen recognized by T cells-1/2 (Melan-A/MART-1/2), Gp100/pme117, tyrosinase, tyrosine-related protein (TRP) 1 and 2, P. polypeptide, melanocortin 1 receptor (MC1R), and prostate-specific antigen. In some embodiments, at least one T-cell subpopulation is specific to Melan-A/MART-1/2. In some embodiments, at least one T-cell subpopulation is specific to Gp100/pme117. In some embodiments, at least one T-cell subpopulation is specific to tyrosinase. In some embodiments, at least one T-cell subpopulation is specific to TRP1. In some embodiments, at least one T-cell subpopulation is specific to TRP2. In some embodiments, at least one T-cell subpopulation is specific to P. polypeptide. In some embodiments, at least one T-cell subpopulation is specific to MC1R. In some embodiments, at least one T-cell subpopulation is specific to prostate-specific antigen.

In some embodiments, a T-cell subpopulation comprising the MUSTANG composition is specific to a mutated TAA selected from a group consisting of β-catenin, breast cancer antigen (BRCA) 1/2, cyclin-dependent kinase (CDK) 4, chronic myelogenous leukemia antigen (CML) 66, fibronectin, MART-2, p53, Ras, TGF-βRII, and truncated epithelial growth factor (tEGFR). In some embodiments, at least one T-cell subpopulation is specific to β-catenin. In some embodiments, at least one T-cell subpopulation is specific to BRCA1. In some embodiments, at least one T-cell subpopulation is specific to BRCA2. In some embodiments, at least one T-cell subpopulation is specific to CDK4. In some embodiments, at least one T-cell subpopulation is specific to CML66. In some embodiments, at least one T-cell subpopulation is specific to fibronectin. In some embodiments, at least one T-cell subpopulation is specific to MART-2. In some embodiments, at least one T-cell subpopulation is specific to p53. In some embodiments, at least one T-cell subpopulation is specific to Ras. In some embodiments, at least one T-cell subpopulation is specific to TGF-βRII. In some embodiments, at least one T-cell subpopulation is specific to tEGFR.

In some embodiments, a T-cell subpopulation comprising the MUSTANG composition is specific to the post-translationally altered TAA mucin (MUC) 1. In some embodiments, at least one T-cell subpopulation is specific to MUC1.

In some embodiments, single antigen T-cell subpopulations are specific to an idiotypic TAA selected from a group consisting of immunoglobulin (Ig) and T cell receptor (TCR). In some embodiments, at least one T-cell subpopulation is specific to Ig. In some embodiments, at least one T-cell subpopulation is specific to TCR.

In some embodiments, a T-cell subpopulation comprising the MUSTANG composition is specific to BCMA. In some embodiments, at least one T-cell subpopulation is specific to BCMA.

In some embodiments, a T-cell subpopulation comprising the MUSTANG composition is specific to CS1. In some embodiments, at least one T-cell subpopulation is specific to CS1.

In some embodiments, a T-cell subpopulation comprising the MUSTANG composition is specific to XBP-1. In some embodiments, at least one T-cell subpopulation is specific to XBP-1.

In some embodiments, a T-cell subpopulation comprising the MUSTANG composition is specific to CD138. In some embodiments, at least one T-cell subpopulation is specific to CD138.

In some embodiments, the MUSTANG composition comprises two or more T-cell subpopulations specific to BCMA, CS1, XBP-1, or CD138.

In certain embodiments, the MUSTANG composition includes two or more T-cell subpopulations directed against WT1, PRAME, Survivin, NY-ESO-1, MAGE-A3, MAGE-A4, Pr3, Cyclin A1, SSX2, Neutrophil Elastase (NE), HPV E6. HPV E7, EBV LMP1, EBV LMP2, EBV EBNA1, and EBV EBNA2. In some embodiments, the MUSTANG composition includes one or more T-cell subpopulations targeting WT1, PRAME, and Survivin.

Wilms tumor gene (WT1) is found in post-natal kidney, pancreas, fat, gonads and hematopoietic stem cells (Chau et al., Trends in Genetics (2012) 28 (10) 515-524). In healthy hematopoietic stem cells WT1 encodes a transcription factor, which regulates cell proliferation, cell death and differentiation (Scharnhorst et al., Gene (2001) 273 (2) 141-161). In recovering marrow, WT1 is expressed to a greater degree than in homeostasis (Boublikova et al., Leukemia (2006) 20 (2) 254-263). Despite the expression of WT1 in healthy stem cells and recovering marrow states, studies to date using antisense or directed cytotoxic therapy against this antigen have not revealed adverse effects on the healthy stem cell population (Rosenfeld et al., Leukemia (2003) 17 (7) 1301-1312).

WT1 is overexpressed in Wilms tumor, soft tissue sarcomas including rhabdomyosarcoma (91.7%) and malignant peripheral nerve sheath tumor (71.4%), ovarian and prostate and cancers (Lee et al., Experimental Cell Research (2001) 264 (1) 74-99; Barbolina et al., Cancer (2008) 112 (7) 1632-1641; Kim et al., World journal of surg one (2014) 12:214; Brett et al., Molecular Cancer (2013) 12:3). In ovarian cancer WT1 expression was frequently identified in primary tumors and was retained in paired peritoneal metastases. WT1 expression in prostate cancer was associated with high-grade disease and may play a role in migration and metastasis. The WT1 gene was initially identified as a tumor suppressor gene due to its inactivation in Wilms' tumor (nephroblastoma), the most common pediatric kidney tumor. However, recent findings have shown that WT1 acts as an oncogene in ovarian and other tumors. In addition, several studies have reported that high expression of WT1 correlates with the aggressiveness of cancers and a poor outcome in leukemia, breast cancer, germ-cell tumor, prostate cancer, soft tissue sarcomas, rhabdomyosarcoma and head and neck squamous cell carcinoma. There are several studies describing WT1 expression in ovarian cancers. A positive expression has been primarily observed in serous adenocarcinoma, and WT1 is more frequently expressed in high-grade serous carcinoma, which stands-out from other sub-types due to its aggressive nature and because it harbors unique genetic alterations. Patients with WT1-positive tumors tend to have a higher grade and stage of tumor.

Preferentially expressed antigen of melanoma (PRAME), initially identified in melanoma, has been associated with other tumors including neuroblastoma, osteosarcoma, soft tissue sarcomas, head and neck, lung and renal cancer including Wilms tumor. In neuroblastoma and osteosarcoma, PRAME expression was associated with advanced disease and a poor prognosis. PRAME is also highly expressed in leukemic cells and its expression levels are correlated with relapse and remission. The function in healthy tissue is not well understood, although studies suggest PRAME is involved in proliferation and survival in leukemia cells (Yin Leukemia Research (2011) 35 (9) 1159-1160).

In neuroblastoma PRAME expression was detected in 93% of all patients and in 100% of patients with advanced disease. There was a highly significant association of PRAME expression with both higher tumor stage and the age of patients at diagnosis, both high-risk features (Oberthuer et al., Clinical Cancer Research (2004) 10 (13) 4307-4313). Approximately 70% of osteosarcoma patient specimens expressed PRAME and high expression was associated with poor prognosis and pulmonary metastatic disease (Tan et al., Biochemical and biophysical research communications (2012) 419 (4) 801-808; Toledo et al., Journal of ortho sci (2011) 16 (4) 458-466; Segal et al., Cancer Immunity (2005) 5:4). Soft tissue sarcomas such as synovial cell sarcoma, myxoid/round cell liposarcoma, and malignant fibrous histiocytoma also have been found to express PRAME Segal et al., Cancer Immunity (2005) 5:4).

Survivin is a protein that regulates apoptosis and proliferation of hematopoietic stem cells. While expressed highly during normal fetal development, in most mature tissues, expression is absent, with the exception of possible low-level expression in healthy hematopoietic stem cells (Shinozawa et al., Leukemia Research (2000) 24 (11) 965-970).

Survivin is highly expressed in most cancers including esophageal, non-small-cell lung cancer, central nervous system tumors, breast cancer, colorectal cancer, melanoma, gastric cancer, sarcomas, osteosarcoma, pancreatic cancer, oral cancer, cervical cancer, hepatocellular carcinoma and hematologic malignancies (Fukuda et al., Molecular Cancer Therapeutics (2006) 5 (5) 1087-1098; Tamm et al., Cancer research (1998) 58 (23) 5315-5320; Coughlin et al. Journal of Clin Onc (2006) 24 (36) 5725-5734). Survivin expression has been detected uniformly in neuroblastoma tumor cells (Coughlin et al. Journal of Clin Onc (2006) 24 (36) 5725-5734).

Survivin has been associated with chemotherapy resistant disease, increased tumor recurrence, and poor patient survival. Targeted therapy against the surviving antigen is an attractive cancer treatment strategy (Fukuda et al., Molecular Cancer Therapeutics (2006) 5 (5) 1087-1098).

### Generation of Targeted Tumor-Associated Antigen Peptides for Use in Activating T-Cell Subpopulations

T-cell subpopulations targeting a single TAA can be prepared by pulsing antigen presenting cells or artificial antigen presenting cells with a single peptide or epitope, several peptides or epitopes, or with overlapping peptide libraries of the selected antigen, that for example, include peptides that are about 7, 8, 9, 10, 11, 12, 13, 14, 15, 16 or more amino acids long and overlapping one another by 5, 6, 7, 8, 9, 10, 11 or more amino acids, in certain aspects. GMP-quality overlapping peptide libraries directed to a number of tumor-associated antigens are commercially available, for example, through JPT Technologies and/or Miltenyi Biotec. In particular embodiments, the peptides are 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and there is overlap of 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length.

In some embodiments, the T-cell subpopulation is specific to one or more known epitopes of the targeted single TAA. Much work has been done to determine specific epitopes of TAAs and the HLA alleles they are associated with. Non-limiting examples of specific epitopes of TAAs and the alleles they are associated with can be found in Kessler et al., J Exp Med. 2001 Jan. 1; 193(1):73-88; Oka et al. Immunogenetics. 2000 February; 51(2):99-107; Ohminami et al., Blood. 2000 Jan. 1; 95(1):286-93; Schmitz et al., Cancer Res. 2000 Sep. 1; 60(17):4845-9 and Bachinsky et al., Cancer Immun. 2005 Mar. 22; 5:6, which are each incorporated herein by reference.

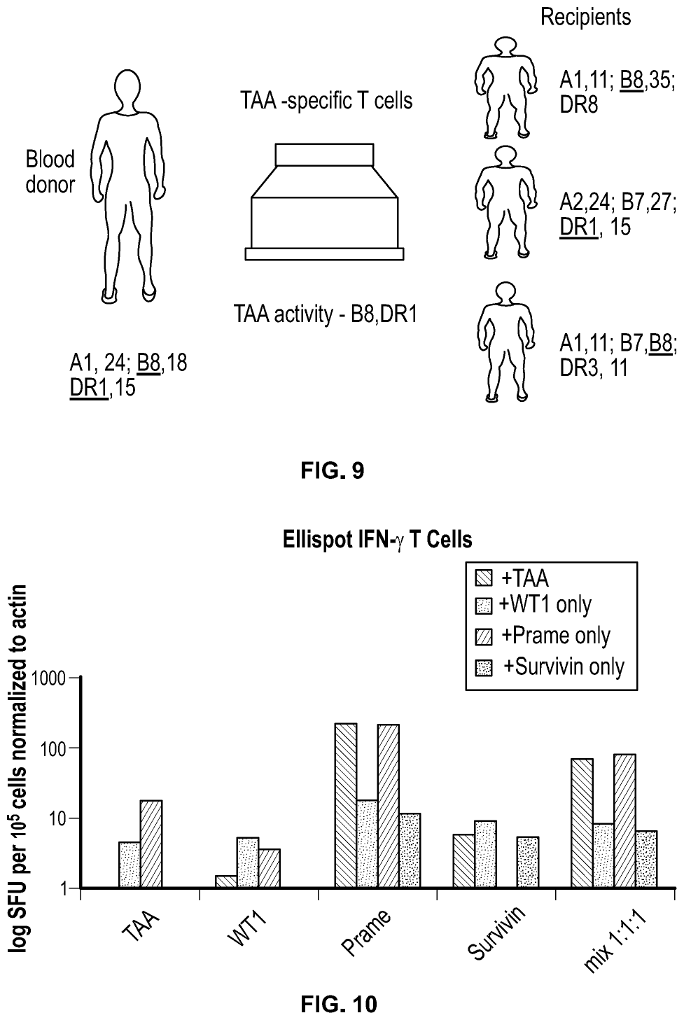

In some embodiments, the TAA peptides used to prime and expand a T-cell subpopulation includes specifically selected HLA-restricted peptides generated by determining the HLA profile of the donor source, and including peptides derived from the targeted TAA that best match the donor's HLA type. By including specifically selected donor HLA-restricted peptides in the peptide mix for priming and expanding T-cell subpopulations, a T-cell subpopulation can be generated that provides greater TAA targeted activity through more than one donor HLA, improving potential efficacy of the T-cell subpopulation. In addition, by generating a T-cell subpopulation with TAA targeted activity through more than one donor HLA allele, a single donor T-cell subpopulation may be included in a MUSTANG composition for multiple recipients with different HLA profiles by matching one or more donor HLAs showing TAA-activity (see, for example, Example 5 and FIG. 9). In some embodiments, the TAA peptides used to prime and expand a T-cell subpopulation are derived from HLA-restricted peptides selected from at least one or more of an HLA-A restricted peptide, HLA-B restricted peptide, or HLA-DR restricted peptide. In some embodiments, the HLA-restricted epitopes are specific to at least one or more of a cell donor's HLA-A alleles, HLA-B alleles, or HLA-DR alleles. In some embodiments, the HLA-A alleles are selected from a group comprising HLA-A*01, HLA-A*02:01, HLA-A*03, HLA-A*11:01, HLA-A*24:02, HLA-A*26, or HLA-A*68:01. In some embodiments, the HLA-B alleles are selected from a group comprising HLA-B*07:02, HLA-B*08, HLA-B*15:01 (B62), HLA-B*18, HLA-B*27:05, HLA-B*35:01, or HLA-B*58:02. In some embodiments, the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b). Suitable methods for generating HLA-restricted peptides from an antigen have been described in, for example, Rammensee, H G., Bachmann, J., Emmerich, N. et al., SYFPEITHI: database for MEW ligands and peptide motifs. Immunogenetics (1999) 50: 213. https://doi.org/10.1007/s002510050595. In some embodiments, the mastermix of peptides includes both an overlapping peptide library and specifically selected HLA-restricted peptides generated by determining the HLA profile of the donor source.

This focused approach to activation can increase the effectiveness of the activated T-cell subpopulation, and ultimately, the MUSTANG composition. While the prior art taught that one can enrich a peptide mixture with an epitope in a multi-tumor-associated antigen approach, this invention provides a new platform for optimizing therapy by targeted activation of T-cell subpopulations with peptides that are most likely to cause activation, and can each be tested for confirmation, prior to being combined in the MUSTANG composition.

WT-1 Antigenic Peptides

In some embodiments, the MUSTANG composition includes WT-1 specific T-cells. WT1 specific T-cells can be generated as described below using one or more antigenic peptides to WT1. In some embodiments, the WT1 specific T-cells are generated using one or more antigenic peptides to WT1, or a modified or heteroclitic peptide derived from a WT1 peptide. In some embodiments, WT1 specific T-cells are generated using a WT1 antigen library comprising a pool of peptides (for example 15mers) containing amino acid overlap (for example 11 amino acids of overlap) between each sequence formed by scanning the protein amino acid sequence SEQ. ID. No. 1 (UniProtKB-P19544 (WT1_HUMAN)):

MGSDVRDLNALLPAVPSLGGGGGCALPVSGAAQWAPVLDFAPPGASAYGS
LGGPAPPPAPPPPPPPPPHSFIKQEPSWGGAEPHEEQCLSAFTVHFSGQF
TGTAGACRYGPFGPPPPSQASSGQARMFPNAPYLPSCLESQPAIRNQGYS
TVTEDGTPSYGHTPSHHAAQFPNHSFKHEDPMGQQGSLGEQQYSVPPPVY
GCHTPTDSCTGSQALLLRTPYSSDNLYQMTSQLECMTWNQMNLGATLKGV
AAGSSSSVKWTEGQSNHSTGYESDNHTTPILCGAQYRIHTHGVERGIQDV
RRVPGVAPTLVRSASETSEKRPFMCAYPGCNKRYFKLSHLQMHSRKHTGE
KPYQCDFKDCERRFSRSDQLKRHQRRHTGVKPFQCKTCQRKFSRSDHLKT
HTRTHTGKTSEKPFSCRWPSCQKKFARSDELVRHHNMHQRNMTKLQLAL

The antigenic library is commercially available, for example, from JPT (Product Code: PM-WT1: Pep Mix′ Human (WT1/WT33)). In some embodiments, the WT1 specific T-cells are generated using a commercially available overlapping antigenic library made up of WT1 peptides.

In some embodiments, the WT1 specific T-cells are generated using one or more antigenic peptides to WT1, or a modified or heteroclitic peptide derived from a WT1 peptide,

In some embodiments, the WT1 specific T-cells are generated using one or more antigenic peptides to WT1, or a modified or heteroclitic peptide derived from a WT1 peptide. In some embodiments, the WT1 specific T-cells are generated with peptides that recognize class I MHC molecules. In some embodiments, the WT1 specific T-cells are generated with peptides that recognize class II MHC molecules. In some embodiments, the WT1 specific T-cells are generated with peptides that recognize both class I and class II MHC molecules.

In some embodiments, the WT1 specific T-cells are generated with peptides that recognize class I MHC molecules. In some embodiments, the WT1 specific T-cells are generated with peptides that recognize class II MHC molecules. In some embodiments, the WT1 specific T-cells are generated with peptides that recognize both class I and class II MHC molecules.

In some embodiments, the WT1 peptides used to prime and expand a T-cell subpopulation includes specifically selected HLA-restricted peptides generated by determining the HLA profile of the donor source, and including peptides derived from WT1 that best match the donor's HLA. In some embodiments, the WT1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting WT1 derived, wherein the T-cell subpopulation is primed and expanded using a group of peptides that are HLA-restricted to the donor's HLA profile. In certain embodiments, the T-cell subpopulation is exposed to a peptide mix that includes one ore more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 1-7, the HLA-B peptides are selected from the peptides of Tables 8-14, and the HLA-DR peptides are selected from the peptides of Tables 15-20. For example, if the donor cell source has an HLA profile that is HLA-A*01/*02:01; HLA-B*15:01/*18; and HLA-DRB1*0101/*0301, then the WT1 peptides used to prime and expand the WT1 specific T-cell subpopulation are restricted to the specific HLA profile, and may include the peptides identified in Table 1 (Seq. ID. Nos. 2-11) for HLA-A*01; Table 2 (Seq. ID. No. 12-21) for HLA-A*02:01; Table 10 (Seq. ID. No. 92-101) for HLA-B*15:01; Table 11 (Seq. ID. No. 102-111) for HLA-B*18; Table 15 (Seq. ID. No. 142-151) for HLA-DRB1*0101; and Table 16 (Seq. ID. No. 152-159) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the WT1 targeted T-cell subpopulation is primed and expanded with one or more WT1-derived peptides selected from Table 1 (Seq. ID. Nos. 2-11). In some embodiments, the donor cell source is HLA-A*01, and the WT1 targeted T-cell subpopulation is primed and expanded with WT1-derived peptides selected from Table 1 (Seq. ID. Nos. 2-11). In some embodiments, the donor cell source is HLA-A*01, and the WT1 targeted T-cell subpopulation is primed and expanded with WT1-derived peptides comprising the peptides of Table 1 (Seq. ID. Nos. 2-11). In some embodiments, the donor cell source is HLA-A*01, and the WT1 targeted T-cell subpopulation is primed and expanded with WT1-derived peptides comprising the peptides of Table 1 (Seq. ID. Nos. 2-11) and at least one additional set of peptides based on the donor cell source HLA-A profile, wherein the at least one additional set of peptides are selected from the peptides of Tables 2-7. In some embodiments, the WT1-derived peptides also include one or more sets of HLA-B and HLA-DR restricted peptides selected from Tables 8-20 (Seq. ID Nos. 72-198).

TABLE 1
WT1 HLA-A*01 Epitope Peptides

| SEQ ID NO. | Sequence |
|---|---|
| 2 | TSEKRPFMCAY |
| 3 | STVTFDGTPSY |
| 4 | HTTPILCGAQY |
| 5 | ESQPAIRNQGY |
| 6 | GSQALLLRTPY |
| 7 | HSRKHTGEKPY |
| 8 | FTGTAGACRY |
| 9 | RTPYSSDNLY |
| 10 | TTPILCGAQY |
| 11 | VTFDGTPSY |

In some embodiments, the donor cell source is HLA-A*02:01, and the WT1 targeted T-cell subpopulation is primed and expanded with one or more WT1-derived peptides selected from Table 2 (Seq. ID. Nos. 12-21). In some embodiments, the donor cell source is HLA-A*02:01, and the WT1 targeted T-cell subpopulation is primed and expanded with WT1-derived peptides selected from Table 2 (Seq. ID. Nos. 12-21). In some embodiments, the donor cell source is HLA-A*02:01, and the WT1 targeted T-cell subpopulation is primed and expanded with WT1-derived peptides comprising the peptides of Table 2 (Seq. ID. Nos. 12-21). In some embodiments, the donor cell source is HLA-A*02:01, and the WT1 targeted T-cell subpopulation is primed and expanded with WT1-derived peptides comprising the peptides of Table 2 (Seq. ID. Nos. 12-21) and at least one additional set of peptides based on the donor cell source HLA-A profile, wherein the at least one additional set of peptides are selected from the peptides of Tables 1, and 3-7. In some embodiments, the WT1-derived peptides also include one or more sets of HLA-B and HLA-DR restricted peptides selected from Tables 8-20 (Seq. ID Nos. 72-198).

TABLE 2
WT1 HLA-A*02:01 Epitope Peptides

| SEQ ID NO. | Sequence |
|---|---|
| 12 | SLGGGGGCAL |
| 13 | NALLPAVPSL |
| 14 | AIRNQGYSTV |
| 15 | NMHQRNMTKL |
| 16 | ALLPAVPSL |
| 17 | DLNALLPAV |
| 18 | SLGEQQYSV |
| 19 | NLGATLKGV |
| 20 | NLYQMTSQL |
| 21 | ILCGAQYRI |

In some embodiments, the donor cell source is HLA-A*03, and the WT1 targeted T-cell subpopulation is primed and expanded with one or more WT1-derived peptides selected from Table 3 (Seq. ID. Nos. 22-31). In some embodiments, the donor cell source is HLA-A*03, and the WT1 targeted T-cell subpopulation is primed and expanded with WT1-derived peptides selected from Table 3 (Seq. ID. Nos. 22-31). In some embodiments, the donor cell source is HLA-A*03, and the WT1 targeted T-cell subpopulation is primed and expanded with WT1-derived peptides comprising the peptides of Table 3 (Seq. ID. Nos. 22-31). In some embodiments, the donor cell source is HLA-A*03, and the WT1 targeted T-cell subpopulation is primed and expanded with WT1-derived peptides comprising the peptides of Table 3 (Seq. ID. Nos. 22-31) and at least one additional set of peptides based on the donor cell source HLA-A profile, wherein the at least one additional set of peptides are selected from the peptides of Tables 1-2 and 4-7. In some embodiments, the WT1-derived peptides also include one or more sets of HLA-B and HLA-DR restricted peptides selected from Tables 8-20 (Seq. ID Nos. 72-198).

TABLE 3
WT1 HLA-A*03 Epitope Peptides

| SEQ ID NO. | Sequence |
|---|---|
| 22 | DVRRVPGVAP |
| 23 | ALLPAVPSLG |
| 24 | ALPVSGAAQW |
| 25 | AIRNQGYSTV |
| 26 | RHQRRHTGVK |
| 27 | GVFRGIQDVR |
| 28 | RVPGVAPTL |
| 29 | RIHTHGVFR |
| 30 | DVRRVPGVA |
| 31 | HQRRHTGVK |

In some embodiments, the donor cell source is HLA-A*11:01, and the WT1 targeted T-cell subpopulation is primed and expanded with one or more WT1-derived peptides selected from Table 4 (Seq. ID. Nos. 32-41). In some embodiments, the donor cell source is HLA-A*11:01, and the WT1 targeted T-cell subpopulation is primed and expanded with WT1-derived peptides selected from Table 4 (Seq. ID. Nos. 32-41). In some embodiments, the donor cell source is HLA-A*11:01, and the WT1 targeted T-cell subpopulation is primed and expanded with WT1-derived peptides comprising the peptides of Table 4 (Seq. ID. Nos. 32-41). In some embodiments, the donor cell source is HLA-A*11:01, and the WT1 targeted T-cell subpopulation is primed and expanded with WT1-derived peptides comprising the peptides of Table 4 (Seq. ID. Nos. 32-41) and at least one additional set of peptides based on the donor cell source HLA-A profile, wherein the at least one additional set of peptides are selected from the peptides of Tables 1-3 and 5-7. In some embodiments, the WT1-derived peptides also include one or more sets of HLA-B and HLA-DR restricted peptides selected from Tables 8-20 (Seq. ID Nos. 72-198).

TABLE 4
WT1 HLA-A*11:01 Epitope Peptides

| SEQ ID NO. | Sequence |
|---|---|
| 32 | CTGSQALLLR |
| 33 | GVFRGIQDVR |
| 34 | HTGVKPFQCK |
| 35 | RTHTGKTSEK |
| 36 | KTHTRTHTGK |
| 37 | RSASETSEKR |
| 38 | LSHLQMHSRK |
| 39 | FSCRWPSCQK |
| 40 | RSASETSEK |
| 41 | FSRSDQLKR |

In some embodiments, the donor cell source is HLA-A*24:02, and the WT1 targeted T-cell subpopulation is primed and expanded with one or more WT1-derived peptides selected from Table 5 (Seq. ID. Nos. 42-51). In some embodiments, the donor cell source is HLA-A*24:02, and the WT1 targeted T-cell subpopulation is primed and expanded with WT1-derived peptides selected from Table 5 (Seq. ID. Nos. 42-51). In some embodiments, the donor cell source is HLA-A*24:02, and the WT1 targeted T-cell subpopulation is primed and expanded with WT1-derived peptides comprising the peptides of Table 5 (Seq. ID. Nos. 42-51). In some embodiments, the donor cell source is HLA-A*24:02, and the WT1 targeted T-cell subpopulation is primed and expanded with WT1-derived peptides comprising the peptides of Table 5 (Seq. ID. Nos. 42-51) and at least one additional set of peptides based on the donor cell source HLA-A profile, wherein the at least one additional set of peptides are selected from the peptides of Tables 1-4 and 6-7. In some embodiments, the WT1-derived peptides also include one or more sets of HLA-B and HLA-DR restricted peptides selected from Tables 8-20 (Seq. ID Nos. 72-198).

TABLE 5
WT1 HLA-A*24:02 Epitope Peptides

| SEQ ID NO. | Sequence |
|---|---|
| 42 | AYPGCNKRYF |
| 43 | QYRIHTHGVF |
| 44 | AFTVHFSGQF |
| 45 | PPPPPPPHSF |
| 46 | PPPPPPHSFI |
| 47 | PYLPSCLESQ |
| 48 | DFKDCERRF |
| 49 | GCNKRYFKL |
| 50 | ALLPAVPSL |
| 51 | PPPPPPHSF |

In some embodiments, the donor cell source is HLA-A*26, and the WT1 targeted T-cell subpopulation is primed and expanded with one or more WT1-derived peptides selected from Table 6 (Seq. ID. Nos. 52-61). In some embodiments, the donor cell source is HLA-A*26, and the WT1 targeted T-cell subpopulation is primed and expanded with WT1-derived peptides selected from Table 6 (Seq. ID. Nos. 52-61). In some embodiments, the donor cell source is HLA-A*26, and the WT1 targeted T-cell subpopulation is primed and expanded with WT1-derived peptides comprising the peptides of Table 6 (Seq. ID. Nos. 52-61). In some embodiments, the donor cell source is HLA-A*26, and the WT1 targeted T-cell subpopulation is primed and expanded with WT1-derived peptides comprising the peptides of Table 6 (Seq. ID. Nos. 52-61) and at least one additional set of peptides based on the donor cell source HLA-A profile, wherein the at least one additional set of peptides are selected from the peptides of Tables 1-5 and 7. In some embodiments, the WT1-derived peptides also include one or more sets of HLA-B and HLA-DR restricted peptides selected from Tables 8-20 (Seq. ID Nos. 72-198).

TABLE 6
WT1 HLA-A*26 Epitopes Peptides

| SEQ ID NO. | Sequence |
|---|---|
| 52 | TVTFDGTPSY |
| 53 | DFAPPGASAY |
| 54 | EGQSNHSTGY |
| 55 | TTPILCGAQY |
| 56 | ETSEKRPFMC |
| 57 | DVRDLNALL |
| 58 | VTFDGTPSY |
| 59 | FTVHFSGQF |
| 60 | EKRPFMCAY |
| 61 | ETSEKRPFM |

In some embodiments, the donor cell source is HLA-A*68:01, and the WT1 targeted T-cell subpopulation is primed and expanded with one or more WT1-derived peptides selected from Table 7 (Seq. ID. Nos. 62-71). In some embodiments, the donor cell source is HLA-A*68:01, and the WT1 targeted T-cell subpopulation is primed and expanded with WT1-derived peptides selected from Table 7 (Seq. ID. Nos. 62-71). In some embodiments, the donor cell source is HLA-A*68:01, and the WT1 targeted T-cell subpopulation is primed and expanded with WT1-derived peptides comprising the peptides of Table 7 (Seq. ID. Nos. 62-71). In some embodiments, the donor cell source is HLA-A*68:01, and the WT1 targeted T-cell subpopulation is primed and expanded with WT1-derived peptides comprising the peptides of Table 7 (Seq. ID. Nos. 62-71) and at least one additional set of peptides based on the donor cell source HLA-A profile, wherein the at least one additional set of peptides are selected from the peptides of Tables 1-6. In some embodiments, the WT1-derived peptides also include one or more sets of HLA-B and HLA-DR restricted peptides selected from Tables 8-20 (Seq. ID Nos. 72-198).

TABLE 7
WT1 HLA-A*68:01 Epitope Peptides

| SEQ ID NO. | Sequence |
|---|---|
| 62 | GVFRGIQDVRR |
| 63 | TTPILCGAQYR |
| 64 | ELVRHHNMHQR |
| 65 | PSCLESQPAIR |
| 66 | CTGSQALLLR |
| 67 | GVFRGIQDVR |
| 68 | KTHTRTHTGK |
| 69 | LVRHHNMHQR |
| 70 | FTGTAGACR |
| 71 | RIHTHGVFR |

In some embodiments, the donor cell source is HLA-B*07:02, and the WT1 targeted T-cell subpopulation is primed and expanded with one or more WT1-derived peptides selected from Table 8 (Seq. ID. Nos. 72-81). In some embodiments, the donor cell source is HLA-B*07:02, and the WT1 targeted T-cell subpopulation is primed and expanded with WT1-derived peptides selected from Table 8 (Seq. ID. Nos. 72-81). In some embodiments, the donor cell source is HLA-B*07:02, and the WT1 targeted T-cell subpopulation is primed and expanded with WT1-derived peptides comprising the peptides of Table 8 (Seq. ID. Nos. 72-81). In some embodiments, the donor cell source is HLA-B*07:02, and the WT1 targeted T-cell subpopulation is primed and expanded with WT1-derived peptides comprising the peptides of Table 8 (Seq. ID. Nos. 72-81) and at least one additional set of peptides based on the donor cell source HLA-B profile, wherein the at least one additional set of peptides are selected from the peptides of Tables 9-14. In some embodiments, the WT1-derived peptides also include one or more sets of HLA-A and HLA-DR restricted peptides selected from Tables 1-7 and 15-20 (Seq. ID Nos. 1-71 and 142-198).

TABLE 8
WT1 HLA-B*07:02 Epitope Peptides

| SEQ ID NO. | Sequence |
|---|---|
| 72 | PPGASAYGSL |
| 73 | EPHEEQCLSA |
| 74 | LPSCLESQPA |
| 75 | PPPPPPHSFI |
| 76 | PPSQASSGQA |
| 77 | DPMGQQGSL |
| 78 | PPPPPHSFI |
| 79 | PPPPPPHSF |
| 80 | TPSHHAAQF |
| 81 | WPSCQKKFA |

In some embodiments, the donor cell source is HLA-B*08, and the WT1 targeted T-cell subpopulation is primed and expanded with one or more WT1-derived peptides selected from Table 9 (Seq. ID. Nos. 82-91). In some embodiments, the donor cell source is HLA-B*08, and the WT1 targeted T-cell subpopulation is primed and expanded with WT1-derived peptides selected from Table 9 (Seq. ID. Nos. 82-91). In some embodiments, the donor cell source is HLA-B*08, and the WT1 targeted T-cell subpopulation is primed and expanded with WT1-derived peptides comprising the peptides of Table 9 (Seq. ID. Nos. 82-91). In some embodiments, the donor cell source is HLA-B*08, and the WT1 targeted T-cell subpopulation is primed and expanded with WT1-derived peptides comprising the peptides of Table 9 (Seq. ID. Nos. 82-91) and at least one additional set of peptides based on the donor cell source HLA-B profile, wherein the at least one additional set of peptides are selected from the peptides of Tables 8 and 10-14. In some embodiments, the WT1-derived peptides also include one or more sets of HLA-A and HLA-DR restricted peptides selected from Tables 1-7 and 15-20 (Seq. ID Nos. 1-71 and 142-198).

TABLE 9
WT1 HLA-B*08 Epitope Peptides

| SEQ ID NO. | Sequence |
|---|---|
| 82 | KRYFKLSHL |
| 83 | GCNKRYFKL |
| 84 | KKFARSDEL |
| 85 | GATLKGVAA |
| 86 | RRFSRSDQL |
| 87 | MTKLQLAL |
| 88 | EPHEEQCL |
| 89 | ETSEKRPF |
| 90 | CNKRYFKL |
| 91 | RNMTKLQL |

In some embodiments, the donor cell source is HLA-B*15:01, and the WT1 targeted T-cell subpopulation is primed and expanded with one or more WT1-derived peptides selected from Table 10 (Seq. ID. Nos. 92-101). In some embodiments, the donor cell source is HLA-B*15:01, and the WT1 targeted T-cell subpopulation is primed and expanded with WT1-derived peptides selected from Table 10 (Seq. ID. Nos. 92-101). In some embodiments, the donor cell source is HLA-B*15:01, and the WT1 targeted T-cell subpopulation is primed and expanded with WT1-derived peptides comprising the peptides of Table 10 (Seq. ID. Nos. 92-101). In some embodiments, the donor cell source is HLA-B*15:01, and the WT1 targeted T-cell subpopulation is primed and expanded with WT1-derived peptides comprising the peptides of Table 10 (Seq. ID. Nos. 92-101) and at least one additional set of peptides based on the donor cell source HLA-B profile, wherein the at least one additional set of peptides are selected from the peptides of Tables 8-9 and 11-14. In some embodiments, the WT1-derived peptides also include one or more sets of HLA-A and HLA-DR restricted peptides selected from Tables 1-7 and 15-20 (Seq. ID Nos. 1-71 and 142-198).

TABLE 10
WT1 HLA-B*15:01 (B62) Epitope Peptides

| SEQ ID NO. | Sequence |
|---|---|
| 92 | QQYSVPPPVY |
| 93 | TVTFDGTPSY |
| 94 | QQGSLGEQQY |
| 95 | SQALLLRTPY |
| 96 | SQPAIRNQGY |
| 97 | FQCKTCQRKF |
| 98 | AQWAPVLDF |
| 99 | GQSNHSTGY |
| 100 | NQGYSTVTF |
| 101 | CLSAFTVHF |

In some embodiments, the donor cell source is HLA-B*18, and the WT1 targeted T-cell subpopulation is primed and expanded with one or more WT1-derived peptides selected from Table 11 (Seq. ID. Nos. 102-111). In some embodiments, the donor cell source is HLA-B*18, and the WT1 targeted T-cell subpopulation is primed and expanded with WT1-derived peptides selected from Table 11 (Seq. ID. Nos. 102-111). In some embodiments, the donor cell source is HLA-B*18, and the WT1 targeted T-cell subpopulation is primed and expanded with WT1-derived peptides comprising the peptides of Table 11 (Seq. ID. Nos. 102-111). In some embodiments, the donor cell source is HLA-B*18, and the WT1 targeted T-cell subpopulation is primed and expanded with WT1-derived peptides comprising the peptides of Table 11 (Seq. ID. Nos. 102-111) and at least one additional set of peptides based on the donor cell source HLA-B profile, wherein the at least one additional set of peptides are selected from the peptides of Tables 8-10 and 12-14. In some embodiments, the WT1-derived peptides also include one or more sets of HLA-A and HLA-DR restricted peptides selected from Tables 1-7 and 15-20 (Seq. ID Nos. 1-71 and 142-198).

TABLE 11
WT1 HLA-B*18 Epitope Peptides

| SEQ ID NO. | Sequence |
|---|---|
| 102 | HEEQCLSAF |
| 103 | SETSEKRPF |
| 104 | GEKPYQCDF |
| 105 | SEKPFSCRW |
| 106 | AEPHEEQCL |
| 107 | DVRDLNALL |
| 108 | QALLLRTPY |
| 109 | EEQCLSAF |
| 110 | ETSEKRPF |
| 111 | DELVRHHN |

In some embodiments, the donor cell source is HLA-B*27:05, and the WT1 targeted T-cell subpopulation is primed and expanded with one or more WT1-derived peptides selected from Table 12 (Seq. ID. Nos. 112-121). In some embodiments, the donor cell source is HLA-B*27:05, and the WT1 targeted T-cell subpopulation is primed and expanded with WT1-derived peptides selected from Table 12 (Seq. ID. Nos. 112-121). In some embodiments, the donor cell source is HLA-B*27:05, and the WT1 targeted T-cell subpopulation is primed and expanded with WT1-derived peptides comprising the peptides of Table 12 (Seq. ID. Nos. 112-121). In some embodiments, the donor cell source is HLA-B*27:05, and the WT1 targeted T-cell subpopulation is primed and expanded with WT1-derived peptides comprising the peptides of Table 12 (Seq. ID. Nos. 112-121) and at least one additional set of peptides based on the donor cell source HLA-B profile, wherein the at least one additional set of peptides are selected from the peptides of Tables 8-11 and 13-14. In some embodiments, the WT1-derived peptides also include one or more sets of HLA-A and HLA-DR restricted peptides selected from Tables 1-7 and 15-20 (Seq. ID Nos. 1-71 and 142-198).

TABLE 12
WT1 HLA-B*27:05 Epitope Peptides

| SEQ ID NO. | Sequence |
|---|---|
| 112 | RRVPGVAPTL |
| 113 | RRFSRSDQLK |
| 114 | CRWPSCQKKF |
| 115 | LRTPYSSDNL |
| 116 | RRFSRSDQL |
| 117 | KRYFKLSHL |
| 118 | RRHTGVKPF |
| 119 | FRGIQDVRR |
| 120 | CRWPSCQKK |
| 121 | ARSDELVRH |

In some embodiments, the donor cell source is HLA-B*35:01, and the WT1 targeted T-cell subpopulation is primed and expanded with one or more WT1-derived peptides selected from Table 13 (Seq. ID. Nos. 122-131). In some embodiments, the donor cell source is HLA-B*35:01, and the WT1 targeted T-cell subpopulation is primed and expanded with WT1-derived peptides selected from Table 13 (Seq. ID. Nos. 122-131). In some embodiments, the donor cell source is HLA-B*35:01, and the WT1 targeted T-cell subpopulation is primed and expanded with WT1-derived peptides comprising the peptides of Table 13 (Seq. ID. Nos. 122-131). In some embodiments, the donor cell source is HLA-B*35:01, and the WT1 targeted T-cell subpopulation is primed and expanded with WT1-derived peptides comprising the peptides of Table 13 (Seq. ID. Nos. 122-131) and at least one additional set of peptides based on the donor cell source HLA-B profile, wherein the at least one additional set of peptides are selected from the peptides of Tables 8-12 and 14. In some embodiments, the WT1-derived peptides also include one or more sets of HLA-A and HLA-DR restricted peptides selected from Tables 1-7 and 15-20 (Seq. ID Nos. 1-71 and 142-198).

TABLE 13
WT1 HLA-B*35:01 Epitope Peptides

| SEQ ID NO. | Sequence |
|---|---|
| 122 | PPGASAYGSL |
| 123 | PPPPPPPHSF |
| 124 | PPPPPPHSFI |
| 125 | TPYSSDNLY |
| 126 | QPAIRNQGY |
| 127 | DPMGQQGSL |
| 128 | TPILCGAQY |
| 129 | TPSHHAAQF |
| 130 | PPPPPPHSF |
| 131 | YPGCNKRYF |

In some embodiments, the donor cell source is HLA-B*58:02, and the WT1 targeted T-cell subpopulation is primed and expanded with one or more WT1-derived peptides selected from Table 14 (Seq. ID. Nos. 132-141). In some embodiments, the donor cell source is HLA-B*58:02, and the WT1 targeted T-cell subpopulation is primed and expanded with WT1-derived peptides selected from Table 14 (Seq. ID. Nos. 132-141). In some embodiments, the donor cell source is HLA-B*58:02, and the WT1 targeted T-cell subpopulation is primed and expanded with WT1-derived peptides comprising the peptides of Table 14 (Seq. ID. Nos. 132-141). In some embodiments, the donor cell source is HLA-B*58:02, and the WT1 targeted T-cell subpopulation is primed and expanded with WT1-derived peptides comprising the peptides of Table 14 (Seq. ID. Nos. 132-141) and at least one additional set of peptides based on the donor cell source HLA-B profile, wherein the at least one additional set of peptides are selected from the peptides of Tables 8-13. In some embodiments, the WT1-derived peptides also include one or more sets of HLA-A and HLA-DR restricted peptides selected from Tables 1-7 and 15-20 (Seq. ID Nos. 1-71 and 142-198).

TABLE 14
WT1 HLA-B*58:02 Epitope Peptides

| SEQ ID NO. | Sequence |
|---|---|
| 132 | ASETSEKRPF |
| 133 | QASSGQARMF |
| 134 | RTPYSSDNLY |
| 135 | DSCTGSQALL |
| 136 | ASSGQARMF |
| 137 | RVPGVAPTL |
| 138 | TSQLECMTW |
| 139 | HTHGVFRGI |
| 140 | RTPYSSDNL |
| 141 | RSDELVRHH |

In some embodiments, the donor cell source is HLA-DRB1*0101, and the WT1 targeted T-cell subpopulation is primed and expanded with one or more WT1-derived peptides selected from Table 15 (Seq. ID. Nos. 142-151). In some embodiments, the donor cell source is HLA-DRB1*0101, and the WT1 targeted T-cell subpopulation is primed and expanded with WT1-derived peptides selected from Table 15 (Seq. ID. Nos. 142-151). In some embodiments, the donor cell source is HLA-DRB1*0101, and the WT1 targeted T-cell subpopulation is primed and expanded with WT1-derived peptides comprising the peptides of Table 15 (Seq. ID. Nos. 142-151). In some embodiments, the donor cell source is HLA-DRB1*0101, and the WT1 targeted T-cell subpopulation is primed and expanded with WT1-derived peptides comprising the peptides of Table 15 (Seq. ID. Nos. 142-151) and at least one additional set of peptides based on the donor cell source HLA-DR profile, wherein the at least one additional set of peptides are selected from the peptides of Tables 16-20. In some embodiments, the WT1-derived peptides also include one or more sets of HLA-A and HLA-B restricted peptides selected from Tables 1-14 (Seq. ID Nos. 1-141).

TABLE 15
WT1 HLA-DRB1*0101 Epitope Peptides

| SEQ ID NO. | Sequence |
|---|---|
| 142 | ASAYGSLGGPAPPPA |
| 143 | GSDVRDLNALLPAVP |
| 144 | IQDVRRVPGVAPTLV |
| 145 | VRDLNALLPAVPSLG |
| 146 | GATLKGVAAGSSSSV |
| 147 | TVHFSGQFTGTAGAC |
| 148 | VRRVPGVAPTLVRSA |
| 149 | NKRYFKLSHLQMHSR |
| 150 | LPAVPSLGGGGGCAL |
| 151 | RDLNALLPAVPSLGG |

In some embodiments, the donor cell source is HLA-DRB1*0301, and the WT1 targeted T-cell subpopulation is primed and expanded with one or more WT1-derived peptides selected from Table 16 (Seq. ID. Nos. 152-159). In some embodiments, the donor cell source is HLA-DRB1*0301, and the WT1 targeted T-cell subpopulation is primed and expanded with WT1-derived peptides selected from Table 16 (Seq. ID. Nos. 152-159). In some embodiments, the donor cell source is HLA-DRB1*0301, and the WT1 targeted T-cell subpopulation is primed and expanded with WT1-derived peptides comprising the peptides of Table 16 (Seq. ID. Nos. 152-159). In some embodiments, the donor cell source is HLA-DRB1*0301, and the WT1 targeted T-cell subpopulation is primed and expanded with WT1-derived peptides comprising the peptides of Table 16 (Seq. ID. Nos. 152-159) and at least one additional set of peptides based on the donor cell source HLA-DR profile, wherein the at least one additional set of peptides are selected from the peptides of Tables 15 and 17-20. In some embodiments, the WT1-derived peptides also include one or more sets of HLA-A and HLA-B restricted peptides selected from Tables 1-14 (Seq. ID Nos. 1-141).

TABLE 16
WT1 HLA-DRB1*0301 Epitope Peptides

| SEQ ID NO. | Sequence |
|---|---|
| 152 | YSTVTFDGTPSYGHT |
| 153 | MGSDVRDLNALLPAV |
| 154 | YQCDFKDCERRFSRS |
| 155 | VPSLGGGGGCALPVS |
| 156 | VLDFAPPGASAYGSL |
| 157 | LYQMTSQLECMTWNQ |
| 158 | PTLVRSASETSEKRP |
| 159 | HHNMHQRNMTKLQLA |

In some embodiments, the donor cell source is HLA-DRB1*0401, and the WT1 targeted T-cell subpopulation is primed and expanded with one or more WT1-derived peptides selected from Table 17 (Seq. ID. Nos. 160-169). In some embodiments, the donor cell source is HLA-DRB1*0401, and the WT1 targeted T-cell subpopulation is primed and expanded with WT1-derived peptides selected from Table 17 (Seq. ID. Nos. 160-169). In some embodiments, the donor cell source is HLA-DRB1*0401, and the WT1 targeted T-cell subpopulation is primed and expanded with WT1-derived peptides comprising the peptides of Table 17 (Seq. ID. Nos. 160-169). In some embodiments, the donor cell source is HLA-DRB1*0401, and the WT1 targeted T-cell subpopulation is primed and expanded with WT1-derived peptides comprising the peptides of Table 17 (Seq. ID. Nos. 160-169) and at least one additional set of peptides based on the donor cell source HLA-DR profile, wherein the at least one additional set of peptides are selected from the peptides of Tables 15-16 and 18-20. In some embodiments, the WT1-derived peptides also include one or more sets of HLA-A and HLA-B restricted peptides selected from Tables 1-14 (Seq. ID Nos. 1-141).

TABLE 17
WT1 HLA-DRB1*0401 (DR4Dw4) Epitope Peptides

| SEQ ID NO. | Sequence |
|---|---|
| 160 | NKRYFKLSHLQMHSR |
| 161 | TVHFSGQFTGTAGAC |
| 162 | ARMFPNAPYLPSCLE |
| 163 | NQGYSTVTFDGTPSY |
| 164 | TPSYGHTPSHHAAQF |
| 165 | NHSFKHEDPMGQQGS |
| 166 | RTPYSSDNLYQMTSQ |
| 167 | SVKWTEGQSNHSTGY |
| 168 | STGYESDNHTTPILC |
| 169 | KRPFMCAYPGCNKRY |

In some embodiments, the donor cell source is HLA-DRB1*0701, and the WT1 targeted T-cell subpopulation is primed and expanded with one or more WT1-derived peptides selected from Table 18 (Seq. ID. Nos. 170-179). In some embodiments, the donor cell source is HLA-DRB1*0701, and the WT1 targeted T-cell subpopulation is primed and expanded with WT1-derived peptides selected from Table 18 (Seq. ID. Nos. 170-179). In some embodiments, the donor cell source is HLA-DRB1*0701, and the WT1 targeted T-cell subpopulation is primed and expanded with WT1-derived peptides comprising the peptides of Table 18 (Seq. ID. Nos. 170-179). In some embodiments, the donor cell source is HLA-DRB1*0701, and the WT1 targeted T-cell subpopulation is primed and expanded with WT1-derived peptides comprising the peptides of Table 18 (Seq. ID. Nos. 170-179) and at least one additional set of peptides based on the donor cell source HLA-DR profile, wherein the at least one additional set of peptides are selected from the peptides of Tables 15-17 and 19-20. In some embodiments, the WT1-derived peptides also include one or more sets of HLA-A and HLA-B restricted peptides selected from Tables 1-14 (Seq. ID Nos. 1-141).

TABLE 18
WT1 HLA-DRB1*0701 Epitope Peptides

| SEQ ID NO. | Sequence |
|---|---|
| 170 | TPSYGHTPSHHAAQF |
| 171 | TVTFDGTPSYGHTPS |
| 172 | LSAFTVHFSGQFTGT |
| 173 | TPTDSCTGSQALLLR |
| 174 | LKGVAAGSSSSVKWT |
| 175 | TVHFSGQFTGTAGAC |
| 176 | YSTVTFDGTPSYGHT |
| 177 | CGAQYRIHTHGVFRG |
| 178 | HGVFRGIQDVRRVPG |
| 179 | APTLVRSASETSEKR |

In some embodiments, the donor cell source is HLA-DRB1*1101, and the WT1 targeted T-cell subpopulation is primed and expanded with one or more WT1-derived peptides selected from Table 19 (Seq. ID. Nos. 180-188). In some embodiments, the donor cell source is HLA-DRB1*1101, and the WT1 targeted T-cell subpopulation is primed and expanded with WT1-derived peptides selected from Table 19 (Seq. ID. Nos. 180-188). In some embodiments, the donor cell source is HLA-DRB1*1101, and the WT1 targeted T-cell subpopulation is primed and expanded with WT1-derived peptides comprising the peptides of Table 19 (Seq. ID. Nos. 180-188). In some embodiments, the donor cell source is HLA-DRB1*1101, and the WT1 targeted T-cell subpopulation is primed and expanded with WT1-derived peptides comprising the peptides of Table 19 (Seq. ID. Nos. 180-188) and at least one additional set of peptides based on the donor cell source HLA-DR profile, wherein the at least one additional set of peptides are selected from the peptides of Tables 15-18 and 20. In some embodiments, the WT1-derived peptides also include one or more sets of HLA-A and HLA-B restricted peptides selected from Tables 1-14 (Seq. ID Nos. 1-141).

TABLE 19
WT1 HLA-DRB1*1101 Epitope Peptides

| SEQ ID NO. | Sequence |
|---|---|
| 180 | FRGIQDVRRVPGVAP |
| 181 | NKRYFKLSHLQMHSR |
| 182 | QCDFKDCERRFSRSD |
| 183 | STGYESDNHTTPILC |
| 184 | SCRWPSCQKKFARSD |
| 185 | AAQWAPVLDFAPPGA |
| 186 | ASAYGSLGGPAPPPA |
| 187 | PGVAPTLVRSASETS |
| 188 | QMNLGATLKGVAAGS |

In some embodiments, the donor cell source is HLA-DRB1*1501, and the WT1 targeted T-cell subpopulation is primed and expanded with one or more WT1-derived peptides selected from Table 20 (Seq. ID. Nos. 189-198). In some embodiments, the donor cell source is HLA-DRB1*1501, and the WT1 targeted T-cell subpopulation is primed and expanded with WT1-derived peptides selected from Table 20 (Seq. ID. Nos. 189-198). In some embodiments, the donor cell source is HLA-DRB1*1501, and the WT1 targeted T-cell subpopulation is primed and expanded with WT1-derived peptides comprising the peptides of Table 20 (Seq. ID. Nos. 189-198). In some embodiments, the donor cell source is HLA-DRB1*1501, and the WT1 targeted T-cell subpopulation is primed and expanded with WT1-derived peptides comprising the peptides of Table 20 (Seq. ID. Nos. 189-198) and at least one additional set of peptides based on the donor cell source HLA-DR profile, wherein the at least one additional set of peptides are selected from the peptides of Tables 15-19. In some embodiments, the WT1-derived peptides also include one or more sets of HLA-A and HLA-B restricted peptides selected from Tables 1-14 (Seq. ID Nos. 1-141).

TABLE 20
WT1 HLA-DRB1*1501 (DR2b) Epitope Peptides

| SEQ ID NO. | Sequence |
|---|---|
| 189 | WAPVLDFAPPGASAY |
| 190 | RPFMCAYPGCNKRYF |
| 191 | GSDVRDLNALLPAVP |
| 192 | NALLPAVPSLGGGGG |
| 193 | PPGASAYGSLGGPAP |
| 194 | EQCLSAFTVHFSGQF |
| 195 | TAGACRYGPFGPPPP |
| 196 | PSCLESQPAIRNQGY |
| 197 | WNQMNLGATLKGVAA |
| 198 | IQDVRRVPGVAPTLV |

PRAME Antigenic Peptides

In some embodiments, the MUSTANG composition includes PRAME specific T-cells. PRAME specific T-cells can be generated as described below using one or more antigenic peptides to PRAME. In some embodiments, the PRAME specific T-cells are generated using one or more antigenic peptides to PRAME, or a modified or heteroclitic peptide derived from a PRAME peptide. In some embodiments, PRAME specific T-cells are generated using a PRAME antigen library comprising a pool of peptides (for example 15mers) containing amino acid overlap (for example 11 amino acids of overlap) between each sequence formed by scanning the protein amino acid sequence SEQ. ID. No. 199 (UniProt KB-P78395) for human melanoma antigen preferentially expressed in tumors (PRAME):

MERRRLWGSIQSRYISMSVWTSPRRLVELAGQSLLKDEALAIAALELLPRE
LEPPLFMAAFDGRHSQTLKAMVQAWPFTCLPLGVLMKGQHLHLETFKAVLD
GLDVLLAQEVRPRRWKLQVLDLRKNSHQDFWTVWSGNRASLYSFPEPEAAQ
PMTKKRKVDGLSTEAEQPFIPVEVLVDLFLKEGACDELFSYLIEKVKRKKN
VLRLCCKKLKIFAMPMQDIKMILKMVQLDSIEDLEVTCTWKLPTLAKFSPY
LGQMINLRRLLLSHIHASSYISPEKEEQYIAQFTSQFLSLQCLQALYVDSL
FFLRGRLDQLLRHVMNPLETLSITNCRLSEGDVMHLSQSPSVSQLSVLSLS
GVMLTDVSPEPLQALLERASATLQDLVFDECGITDDQLLALLPSLSHCSQL
TTLSFYGNSISISALQSLLQHLIGLSNLTHVLYPVPLESYEDIHGTLHLER
LAYLHARLRELLCELGRPSMVWLSANPCPHCGDRTFYDPEPILCPCFMPN

Overlapping antigenic libraries are commercially available, for example, from JPT (Product code: PM-01P4 Pep Mix™ Human (Prame/01P4)). In some embodiments, the PRAME specific T-cells are generated using a commercially available overlapping antigenic library made up of PRAME peptides.

In some embodiments, the PRAME specific T-cells are generated using one or more antigenic peptides to PRAME, or a modified or heteroclitic peptide derived from a PRAME peptide. In some embodiments, the PRAME specific T-cells are generated with peptides that recognize class I MHC molecules. In some embodiments, the PRAME specific T-cells are generated with peptides that recognize class II MHC molecules. In some embodiments, the PRAME specific T-cells are generated with peptides that recognize both class I and class II MHC molecules.

In some embodiments, the PRAME peptides used to prime and expand a T-cell subpopulation includes specifically selected HLA-restricted peptides generated by determining the HLA profile of the donor source, and including peptides derived from PRAME that best match the donor's HLA. In some embodiments, the PRAME peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting PRAME derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 21-27, the HLA-B peptides are selected from the peptides of Tables 28-34, and the HLA-DR peptides are selected from the peptides of Tables 35-40. For example, if the donor cell source has an HLA profile that is HLA-A*01/*02:01; HLA-B*15:01/*18; and HLA-DRB1*0101/*0301, then the PRAME peptides used to prime and expand the PRAME specific T-cell subpopulation are restricted to the specific HLA profile, and may include the peptides identified in Table 21 (Seq. ID. Nos. 200-209) for HLA-A*01; Table 22 (Seq. ID. No. 210-219) for HLA-A*02:01; Table 30 (Seq. ID. No. 289-298) for HLA-B*15:01; Table 31 (Seq. ID. No. 299-308) for HLA-B*18; Table 35 (Seq. ID. No. 339-348) for HLA-DRB1*0101; and Table 36 (Seq. ID. No. 349-358) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the PRAME targeted T-cell subpopulation is primed and expanded with one or more PRAME-derived peptides selected from Table 21 (Seq. ID. Nos. 200-209). In some embodiments, the donor cell source is HLA-A*01, and the PRAME targeted T-cell subpopulation is primed and expanded with PRAME-derived peptides selected from Table 21 (Seq. ID. Nos. 200-209). In some embodiments, the donor cell source is HLA-A*01, and the PRAME targeted T-cell subpopulation is primed and expanded with PRAME-derived peptides comprising the peptides of Table 21 (Seq. ID. Nos. 200-209). In some embodiments, the donor cell source is HLA-A*01, and the PRAME targeted T-cell subpopulation is primed and expanded with PRAME-derived peptides comprising the peptides of Table 21 (Seq. ID. Nos. 200-209) and at least one additional set of peptides based on the donor cell source HLA-A profile, wherein the at least one additional set of peptides are selected from the peptides of Tables 22-27. In some embodiments, the PRAME-derived peptides also include one or more sets of HLA-B and HLA-DR restricted peptides selected from Tables 28-40 (Seq. ID Nos. 269-398).

TABLE 21
PRAME HLA-A*01 Epitope Peptides

| SEQ ID NO. | Sequence |
|---|---|
| 200 | LTDVSPEPLQA |
| 201 | ITDDQLLALLP |
| 202 | HGTLHLERLAY |
| 203 | GTLHLERLAY |
| 204 | CSQLTTLSFY |
| 205 | LSLQCLQALY |
| 206 | PTLAKFSPY |
| 207 | LSNLTHVLY |
| 208 | WSGNRASLY |
| 209 | LSHIHASSY |

In some embodiments, the donor cell source is HLA-A*02:01, and the PRAME targeted T-cell subpopulation is primed and expanded with one or more PRAME-derived peptides selected from Table 22 (Seq. ID. Nos. 210-219). In some embodiments, the donor cell source is HLA-A*02:01, and the PRAME targeted T-cell subpopulation is primed and expanded with PRAME-derived peptides selected from Table 22 (Seq. ID. Nos. 210-219). In some embodiments, the donor cell source is HLA-A*02:01, and the PRAME targeted T-cell subpopulation is primed and expanded with PRAME-derived peptides comprising the peptides of Table 22 (Seq. ID. Nos. 210-219). In some embodiments, the donor cell source is HLA-A*02:01, and the PRAME targeted T-cell subpopulation is primed and expanded with PRAME-derived peptides comprising the peptides of Table 22 (Seq. ID. Nos. 210-219) and at least one additional set of peptides based on the donor cell source HLA-A profile, wherein the at least one additional set of peptides are selected from the peptides of Tables 21, and 23-27. In some embodiments, the PRAME-derived peptides also include one or more sets of HLA-B and HLA-DR restricted peptides selected from Tables 28-40 (Seq. ID Nos. 269-398).

TABLE 22
PRAME HLA-A*02:01 Epitope Peptides

| SEQ ID NO. | Sequence |
|---|---|
| 210 | ALLERASATL |
| 211 | ALAIAALELL |
| 212 | SLSGVMLTDV |
| 213 | ALYVDSLFFL |
| 214 | QLLALLPSL |
| 215 | SLLQHLIGL |
| 216 | RLRELLCEL |
| 217 | YLHARLREL |
| 218 | ALAIAALEL |
| 219 | FLRGRLDQL |

In some embodiments, the donor cell source is HLA-A*03, and the PRAME targeted T-cell subpopulation is primed and expanded with one or more PRAME-derived peptides selected from Table 23 (Seq. ID. Nos. 220-229). In some embodiments, the donor cell source is HLA-A*03, and the PRAME targeted T-cell subpopulation is primed and expanded with PRAME-derived peptides selected from Table 23 (Seq. ID. Nos. 220-229). In some embodiments, the donor cell source is HLA-A*03, and the PRAME targeted T-cell subpopulation is primed and expanded with PRAME-derived peptides comprising the peptides of Table 23 (Seq. ID. Nos. 220-229). In some embodiments, the donor cell source is HLA-A*03, and the PRAME targeted T-cell subpopulation is primed and expanded with PRAME-derived peptides comprising the peptides of Table 23 (Seq. ID. Nos. 220-229) and at least one additional set of peptides based on the donor cell source HLA-A profile, wherein the at least one additional set of peptides are selected from the peptides of Tables 21-22 and 24-27. In some embodiments, the PRAME-derived peptides also include one or more sets of HLA-B and HLA-DR restricted peptides selected from Tables 28-40 (Seq. ID Nos. 269-398).

TABLE 23
PRAME HLA-A*03 Epitope Peptides

| SEQ ID NO. | Sequence |
|---|---|
| 220 | HLIGLSNLTH |
| 221 | RLWGSIQSRY |
| 222 | KVKRKKNVLR |
| 223 | VLYPVPLESY |
| 224 | CLPLGVLMK |
| 225 | ELAGQSLLK |
| 226 | KLQVLDLRK |
| 227 | RLSEGDVMH |
| 228 | YLIEKVKRK |
| 229 | NVLRLCCKK |

In some embodiments, the donor cell source is HLA-A*11:01, and the PRAME targeted T-cell subpopulation is primed and expanded with one or more PRAME-derived peptides selected from Table 24 (Seq. ID. Nos. 230-239). In some embodiments, the donor cell source is HLA-A*11:01, and the PRAME targeted T-cell subpopulation is primed and expanded with PRAME-derived peptides selected from Table 24 (Seq. ID. Nos. 230-239). In some embodiments, the donor cell source is HLA-A*11:01, and the PRAME targeted T-cell subpopulation is primed and expanded with PRAME-derived peptides comprising the peptides of Table 24 (Seq. ID. Nos. 230-239). In some embodiments, the donor cell source is HLA-A*11:01, and the PRAME targeted T-cell subpopulation is primed and expanded with PRAME-derived peptides comprising the peptides of Table 24 (Seq. ID. Nos. 230-239), and at least one additional set of peptides based on the donor cell source HLA-A profile, wherein the at least one additional set of peptides are selected from the peptides of Tables 21-23 and 25-27. In some embodiments, the PRAME-derived peptides also include one or more sets of HLA-B and HLA-DR restricted peptides selected from Tables 28-40 (Seq. ID Nos. 269-398).

TABLE 24
PRAME HLA-A*11:01 Epitope Peptides

| SEQ ID NO. | Sequence |
|---|---|
| 230 | KVKRKKNVLR |
| 231 | PMQDIKMILK |
| 232 | CTWKLPTLAK |
| 233 | AIAALELLPR |
| 234 | AVLDGLDVLL |
| 235 | FSYLIEKVKR |
| 236 | ELAGQSLLK |
| 237 | EVLVDLFLK |
| 238 | ASSYISPEK |
| 239 | ELFSYLIEK |

In some embodiments, the donor cell source is HLA-A*24:02, and the PRAME targeted T-cell subpopulation is primed and expanded with one or more PRAME-derived peptides selected from Table 25 (Seq. ID. Nos. 240-249). In some embodiments, the donor cell source is HLA-A*24:02, and the PRAME targeted T-cell subpopulation is primed and expanded with PRAME-derived peptides selected from Table 25 (Seq. ID. Nos. 240-249). In some embodiments, the donor cell source is HLA-A*24:02, and the PRAME targeted T-cell subpopulation is primed and expanded with PRAME-derived peptides comprising the peptides of Table 25 (Seq. ID. Nos. 240-249). In some embodiments, the donor cell source is HLA-A*24:02, and the PRAME targeted T-cell subpopulation is primed and expanded with PRAME-derived peptides comprising the peptides of Table 25 (Seq. ID. Nos. 240-249), and at least one additional set of peptides based on the donor cell source HLA-A profile, wherein the at least one additional set of peptides are selected from the peptides of Tables 21-24 and 26-27. In some embodiments, the PRAME-derived peptides also include one or more sets of HLA-B and HLA-DR restricted peptides selected from Tables 28-40 (Seq. ID Nos. 269-398).

TABLE 25
PRAME HLA-A*24:02 Epitope Peptides

| SEQ ID NO. | Sequence |
|---|---|
| 240 | QYIAQFTSQF |
| 241 | AYLHARLREL |
| 242 | LFPPLFMAAF |
| 243 | KFSPYLGQMI |
| 244 | FFLRGRLDQL |
| 245 | VSPEPLQALL |
| 246 | SYEDIHGTL |
| 247 | PYLGQMINL |
| 248 | LYVDSLFFL |
| 249 | TFYDPEPIL |

In some embodiments, the donor cell source is HLA-A*26, and the PRAME targeted T-cell subpopulation is primed and expanded with one or more PRAME-derived peptides selected from Table 26 (Seq. ID. Nos. 250-258). In some embodiments, the donor cell source is HLA-A*26, and the PRAME targeted T-cell subpopulation is primed and expanded with PRAME-derived peptides selected from Table 26 (Seq. ID. Nos. 250-258). In some embodiments, the donor cell source is HLA-A*26, and the PRAME targeted T-cell subpopulation is primed and expanded with PRAME-derived peptides comprising the peptides of Table 26 (Seq. ID. Nos. 250-258). In some embodiments, the donor cell source is HLA-A*26, and the PRAME targeted T-cell subpopulation is primed and expanded with PRAME-derived peptides comprising the peptides of Table 26 (Seq. ID. Nos. 250-258) and at least one additional set of peptides based on the donor cell source HLA-A profile, wherein the at least one additional set of peptides are selected from the peptides of Tables 21-25 and 27. In some embodiments, the PRAME-derived peptides also include one or more sets of HLA-B and HLA-DR restricted peptides selected from Tables 28-40 (Seq. ID Nos. 269-398).

TABLE 26
PRAME HLA-A*26 Epitope Peptides

| SEQ ID NO. | Sequence |
|---|---|
| 250 | ETFKAVLDGL |
| 251 | DVSPEPLQAL |
| 252 | ETLSITNCRL |
| 253 | EGACDELFSY |
| 254 | EKEEQYIAQF |
| 255 | SVSQLSVLSL |
| 256 | EVRPRRWKL |
| 257 | ETFKAVLDG |
| 258 | EVLVDLFLK |

In some embodiments, the donor cell source is HLA-A*68:01, and the PRAME targeted T-cell subpopulation is primed and expanded with one or more PRAME-derived peptides selected from Table 27 (Seq. ID. Nos. 259-268). In some embodiments, the donor cell source is HLA-A*68:01, and the PRAME targeted T-cell subpopulation is primed and expanded with PRAME-derived peptides selected from Table 27 (Seq. ID. Nos. 259-268). In some embodiments, the donor cell source is HLA-A*68:01, and the PRAME targeted T-cell subpopulation is primed and expanded with PRAME-derived peptides comprising the peptides of Table 27 (Seq. ID. Nos. 259-268). In some embodiments, the donor cell source is HLA-A*68:01, and the PRAME targeted T-cell subpopulation is primed and expanded with PRAME-derived peptides comprising the peptides of Table 27 (Seq. ID. Nos. 259-268), and at least one additional set of peptides based on the donor cell source HLA-A profile, wherein the at least one additional set of peptides are selected from the peptides of Tables 21-26. In some embodiments, the PRAME-derived peptides also include one or more sets of HLA-B and HLA-DR restricted peptides selected from Tables 28-40 (Seq. ID Nos. 269-398).

TABLE 27
PRAME HLA-A*68:01 Epitope Peptides

| SEQ ID NO. | Sequence |
|---|---|
| 259 | DVLLAQEVRPR |
| 260 | EAAQPMTKKR |
| 261 | KVKRKKNVLR |
| 262 | EAAQPMTKK |
| 263 | EVLVDLFLK |
| 264 | ELFSYLIEK |
| 265 | ETLSITNCR |
| 266 | DVLLAQEVR |
| 267 | DSLFFLRGR |
| 268 | IAALELLPR |

In some embodiments, the donor cell source is HLA-B*07:02, and the PRAME targeted T-cell subpopulation is primed and expanded with one or more PRAME-derived peptides selected from Table 28 (Seq. ID. Nos. 269-278). In some embodiments, the donor cell source is HLA-B*07:02, and the PRAME targeted T-cell subpopulation is primed and expanded with PRAME-derived peptides selected from Table 28 (Seq. ID. Nos. 269-278). In some embodiments, the donor cell source is HLA-B*07:02, and the PRAME targeted T-cell subpopulation is primed and expanded with PRAME-derived peptides comprising the peptides of Table 28 (Seq. ID. Nos. 269-278). In some embodiments, the donor cell source is HLA-B*07:02, and the PRAME targeted T-cell subpopulation is primed and expanded with PRAME-derived peptides comprising the peptides of Table 28 (Seq. ID. Nos. 269-278), and at least one additional set of peptides based on the donor cell source HLA-B profile, wherein the at least one additional set of peptides are selected from the peptides of Tables 29-34. In some embodiments, the PRAME-derived peptides also include one or more sets of HLA-A and HLA-DR restricted peptides selected from Tables 21-27 and 35-40 (Seq. ID Nos. 200-268 and 339-398).

TABLE 28
PRAME HLA-B*07:02 Epitope Peptides

| SEQ ID NO. | Sequence |
|---|---|
| 269 | RPRRWKLQVL |
| 270 | SPSVSQLSVL |
| 271 | LPSLSHCSQL |
| 272 | MPMQDIKMIL |
| 273 | LPRELFPPL |
| 274 | QPFIPVEVL |
| 275 | IPVEVLVDL |
| 276 | SPEPLQALL |
| 277 | RPRRWKLQV |
| 278 | RPSMVWLSA |

In some embodiments, the donor cell source is HLA-B*08, and the PRAME targeted T-cell subpopulation is primed and expanded with one or more PRAME-derived peptides selected from Table 29 (Seq. ID. Nos. 279-288). In some embodiments, the donor cell source is HLA-B*08, and the PRAME targeted T-cell subpopulation is primed and expanded with PRAME-derived peptides selected from Table 29 (Seq. ID. Nos. 279-288). In some embodiments, the donor cell source is HLA-B*08, and the PRAME targeted T-cell subpopulation is primed and expanded with PRAME-derived peptides comprising the peptides of Table 29 (Seq. ID. Nos. 279-288). In some embodiments, the donor cell source is HLA-B*08, and the PRAME targeted T-cell subpopulation is primed and expanded with PRAME-derived peptides comprising the peptides of Table 29 (Seq. ID. Nos. 279-288) and at least one additional set of peptides based on the donor cell source HLA-B profile, wherein the at least one additional set of peptides are selected from the peptides of Tables 28 and 30-34. In some embodiments, the PRAME-derived peptides also include one or more sets of HLA-A and HLA-DR restricted peptides selected from Tables 21-27 and 35-40 (Seq. ID Nos. 200-268 and 339-398).

TABLE 29
PRAME HLA-B*08 Epitope Peptides

| SEQ ID NO. | Sequence |
|---|---|
| 279 | TKKRKVDGL |
| 280 | FLRGRLDQL |
| 281 | KVKRKKNVL |
| 282 | EVRPRRWKL |
| 283 | PRRWKLQVL |
| 284 | VLRLCCKKL |
| 285 | YLHARLREL |
| 286 | RLRELLCEL |
| 287 | HARLRELL |
| 288 | VKRKKNVL |

In some embodiments, the donor cell source is HLA-B*15:01, and the PRAME targeted T-cell subpopulation is primed and expanded with one or more PRAME-derived peptides selected from Table 30 (Seq. ID. Nos. 289-298). In some embodiments, the donor cell source is HLA-B*15:01, and the PRAME targeted T-cell subpopulation is primed and expanded with PRAME-derived peptides selected from Table 30 (Seq. ID. Nos. 289-298). In some embodiments, the donor cell source is HLA-B*15:01, and the PRAME targeted T-cell subpopulation is primed and expanded with PRAME-derived peptides comprising the peptides of Table 30 (Seq. ID. Nos. 289-298). In some embodiments, the donor cell source is HLA-B*15:01, and the PRAME targeted T-cell subpopulation is primed and expanded with PRAME-derived peptides comprising the peptides of Table 30 (Seq. ID. Nos. 289-298) and at least one additional set of peptides based on the donor cell source HLA-B profile, wherein the at least one additional set of peptides are selected from the peptides of Tables 28-29 and 31-34. In some embodiments, the PRAME-derived peptides also include one or more sets of HLA-A and HLA-DR restricted peptides selected from Tables 21-27 and 35-40 (Seq. ID Nos. 200-268 and 339-398).

TABLE 30
PRAME HLA-B*15:01 (B62) Epitope Peptides

| SEQ ID NO. | Sequence |
|---|---|
| 289 | VLYPVPLESY |
| 290 | RLWGSIQSRY |
| 291 | GLSNLTHVLY |
| 292 | RLCCKKLKIF |
| 293 | LLSHIHASSY |
| 294 | TLHLERLAY |
| 295 | GQHLHLETF |
| 296 | SLQCLQALY |
| 297 | ALYVDSLFF |
| 298 | SQLTTLSFY |

In some embodiments, the donor cell source is HLA-B*18, and the PRAME targeted T-cell subpopulation is primed and expanded with one or more PRAME-derived peptides selected from Table 31 (Seq. ID. Nos. 299-308). In some embodiments, the donor cell source is HLA-B*18, and the PRAME targeted T-cell subpopulation is primed and expanded with PRAME-derived peptides selected from Table 31 (Seq. ID. Nos. 299-308). In some embodiments, the donor cell source is HLA-B*18, and the PRAME targeted T-cell subpopulation is primed and expanded with PRAME-derived peptides comprising the peptides of Table 31 (Seq. ID. Nos. 299-308). In some embodiments, the donor cell source is HLA-B*18, and the PRAME targeted T-cell subpopulation is primed and expanded with PRAME-derived peptides comprising the peptides of Table 31 (Seq. ID. Nos. 299-308) and at least one additional set of peptides based on the donor cell source HLA-B profile, wherein the at least one additional set of peptides are selected from the peptides of Tables 28-30 and 32-34. In some embodiments, the PRAME-derived peptides also include one or more sets of HLA-A and HLA-DR restricted peptides selected from Tables 21-27 and 35-40 (Seq. ID Nos. 200-268 and 339-398).

TABLE 31
PRAME HLA-B*18 Epitope Peptides

| SEQ ID NO. | Sequence |
|---|---|
| 299 | DEALAIAAL |
| 300 | LELLPRELF |
| 301 | KEGACDELF |
| 302 | PEPILCPCF |
| 303 | VEVLVDLF |
| 304 | EEQYIAQF |
| 305 | LELLPREL |
| 306 | RELFPPLF |
| 307 | SEGDVMHL |
| 308 | LERASATL |

In some embodiments, the donor cell source is HLA-B*27:05, and the PRAME targeted T-cell subpopulation is primed and expanded with one or more PRAME-derived peptides selected from Table 32 (Seq. ID. Nos. 309-318). In some embodiments, the donor cell source is HLA-B*27:05, and the PRAME targeted T-cell subpopulation is primed and expanded with PRAME-derived peptides selected from Table 32 (Seq. ID. Nos. 309-318). In some embodiments, the donor cell source is HLA-B*27:05, and the PRAME targeted T-cell subpopulation is primed and expanded with PRAME-derived peptides comprising the peptides of Table 32 (Seq. ID. Nos. 309-318). In some embodiments, the donor cell source is HLA-B*27:05, and the PRAME targeted T-cell subpopulation is primed and expanded with PRAME-derived peptides comprising the peptides of Table 32 (Seq. ID. Nos. 309-318) and at least one additional set of peptides based on the donor cell source HLA-B profile, wherein the at least one additional set of peptides are selected from the peptides of Tables 28-31 and 33-34. In some embodiments, the PRAME-derived peptides also include one or more sets of HLA-A and HLA-DR restricted peptides selected from Tables 21-27 and 35-40 (Seq. ID Nos. 200-268 and 339-398).

TABLE 32
PRAME HLA-B*27:05 Epitope Peptides

| SEQ ID NO. | Sequence |
|---|---|
| 309 | RRLWGSIQSR |
| 310 | RRWKLQVLDL |
| 311 | ERLAYLHARL |
| 312 | ARLRELLCEL |
| 313 | KRKKNVLRL |
| 314 | RRLLLSHIH |
| 315 | GRLDQLLRH |
| 316 | PRRWKLQVL |
| 317 | LRLCCKKLK |
| 318 | ERLAYLHAR |

In some embodiments, the donor cell source is HLA-B*35:01, and the PRAME targeted T-cell subpopulation is primed and expanded with one or more PRAME-derived peptides selected from Table 33 (Seq. ID. Nos. 319-328). In some embodiments, the donor cell source is HLA-B*35:01, and the PRAME targeted T-cell subpopulation is primed and expanded with PRAME-derived peptides selected from Table 33 (Seq. ID. Nos. 319-328). In some embodiments, the donor cell source is HLA-B*35:01, and the PRAME targeted T-cell subpopulation is primed and expanded with PRAME-derived peptides comprising the peptides of Table 33 (Seq. ID. Nos. 319-328). In some embodiments, the donor cell source is HLA-B*35:01, and the PRAME targeted T-cell subpopulation is primed and expanded with PRAME-derived peptides comprising the peptides of Table 33 (Seq. ID. Nos. 319-328) and at least one additional set of peptides based on the donor cell source HLA-B profile, wherein the at least one additional set of peptides are selected from the peptides of Tables 28-32 and 34. In some embodiments, the PRAME-derived peptides also include one or more sets of HLA-A and HLA-DR restricted peptides selected from Tables 21-27 and 35-40 (Seq. ID Nos. 200-268 and 339-398).

TABLE 33
PRAME HLA-B*35:01 Epitope Peptides

| SEQ ID NO. | Sequence |
|---|---|
| 319 | RPRRWKLQVL |
| 320 | SPSVSQLSVL |
| 321 | LPRELFPPLF |
| 322 | IPVEVLVDLF |
| 323 | MPMQDIKMIL |
| 324 | LPTLAKFSPY |
| 325 | IPVEVLVDL |
| 326 | LPRELFPPL |
| 327 | SPEPLQALL |
| 328 | QPFIPVEVL |

In some embodiments, the donor cell source is HLA-B*58:02, and the PRAME targeted T-cell subpopulation is primed and expanded with one or more PRAME-derived peptides selected from Table 34 (Seq. ID. Nos. 329-338). In some embodiments, the donor cell source is HLA-B*58:02, and the PRAME targeted T-cell subpopulation is primed and expanded with PRAME-derived peptides selected from Table 34 (Seq. ID. Nos. 329-338). In some embodiments, the donor cell source is HLA-B*58:02, and the PRAME targeted T-cell subpopulation is primed and expanded with PRAME-derived peptides comprising the peptides of Table 34 (Seq. ID. Nos. 329-338). In some embodiments, the donor cell source is HLA-B*58:02, and the PRAME targeted T-cell subpopulation is primed and expanded with PRAME-derived peptides comprising the peptides of Table 34 (Seq. ID. Nos. 329-338) and at least one additional set of peptides based on the donor cell source HLA-B profile, wherein the at least one additional set of peptides are selected from the peptides of Tables 28-33. In some embodiments, the PRAME-derived peptides also include one or more sets of HLA-A and HLA-DR restricted peptides selected from Tables 21-27 and 35-40 (Seq. ID Nos. 200-268 and 339-398).

TABLE 34
PRAME HLA-B*58:02 Epitope Peptides

| SEQ ID NO. | Sequence |
|---|---|
| 329 | MSVWTSPRRL |
| 330 | AALELLPREL |
| 331 | KAVLDGLDVL |
| 332 | LAQEVRPRRW |
| 333 | ESYEDIHGTL |
| 334 | LSLQCLQALY |
| 335 | VSPEPLQALL |
| 336 | LSHCSQLTTL |
| 337 | KAMVQAWPF |
| 338 | KVKRKKNVL |

In some embodiments, the donor cell source is HLA-DRB1*0101, and the PRAME targeted T-cell subpopulation is primed and expanded with one or more PRAME-derived peptides selected from Table 35 (Seq. ID. Nos. 339-348). In some embodiments, the donor cell source is HLA-DRB1*0101, and the PRAME targeted T-cell subpopulation is primed and expanded with PRAME-derived peptides selected from Table 35 (Seq. ID. Nos. 339-348). In some embodiments, the donor cell source is HLA-DRB1*0101, and the PRAME targeted T-cell subpopulation is primed and expanded with PRAME-derived peptides comprising the peptides of Table 35 (Seq. ID. Nos. 339-348). In some embodiments, the donor cell source is HLA-DRB1*0101, and the PRAME targeted T-cell subpopulation is primed and expanded with PRAME-derived peptides comprising the peptides of Table 35 (Seq. ID. Nos. 339-348) and at least one additional set of peptides based on the donor cell source HLA-DR profile, wherein the at least one additional set of peptides are selected from the peptides of Tables 36-40. In some embodiments, the PRAME-derived peptides also include one or more sets of HLA-A and HLA-B restricted peptides selected from Tables 21-34 (Seq. ID Nos. 200-338).

TABLE 35
PRAME HLA-DRB1*0101 Epitope Peptides

| SEQ ID NO. | Sequence |
|---|---|
| 339 | PRRLVELAGQSLLKD |
| 340 | LDGLDVLLAQEVRPR |
| 341 | FLSLQCLQALYVDSL |
| 342 | RHVMNPLETLSITNC |
| 343 | QLSVLSLSGVMLTDV |
| 344 | RRLWGSIQSRYISMS |
| 345 | EEQYIAQFTSQFLSL |
| 346 | DDQLLALLPSLSHCS |
| 347 | GVMLTDVSPEPLQAL |
| 348 | GQSLLKDEALAIAAL |

In some embodiments, the donor cell source is HLA-DRB1*0301, and the PRAME targeted T-cell subpopulation is primed and expanded with one or more PRAME-derived peptides selected from Table 36 (Seq. ID. Nos. 349-358). In some embodiments, the donor cell source is HLA-DRB1*0301, and the PRAME targeted T-cell subpopulation is primed and expanded with PRAME-derived peptides selected from Table 36 (Seq. ID. Nos. 349-358). In some embodiments, the donor cell source is HLA-DRB1*0301, and the PRAME targeted T-cell subpopulation is primed and expanded with PRAME-derived peptides comprising the peptides of Table 36 (Seq. ID. Nos. 349-358). In some embodiments, the donor cell source is HLA-DRB1*0301, and the PRAME targeted T-cell subpopulation is primed and expanded with PRAME-derived peptides comprising the peptides of Table 36 (Seq. ID. Nos. 349-358) and at least one additional set of peptides based on the donor cell source HLA-DR profile, wherein the at least one additional set of peptides are selected from the peptides of Tables 35 and 37-40. In some embodiments, the PRAME-derived peptides also include one or more sets of HLA-A and HLA-B restricted peptides selected from Tables 21-34 (Seq. ID Nos. 200-338).

TABLE 36
PRAME HLA-DRB1*0301 (DR17) Epitope Peptides

| SEQ ID NO. | Sequence |
|---|---|
| 349 | ECGITDDQLLALLPS |
| 350 | LKMVQLDSIEDLEVT |
| 351 | LQALYVDSLFFLRGR |
| 352 | RRLVELAGQSLLKDE |
| 353 | IAALELLPRELFPPL |
| 354 | LGQMINLRRLLLSHI |
| 355 | FWTVWSGNRASLYSF |
| 356 | SSYISPEKEEQYIAQ |
| 357 | LAYLHARLRELLCEL |
| 358 | GQSLLKDEALAIAAL |

In some embodiments, the donor cell source is HLA-DRB1*0401, and the PRAME targeted T-cell subpopulation is primed and expanded with one or more PRAME-derived peptides selected from Table 37 (Seq. ID. Nos. 359-368). In some embodiments, the donor cell source is HLA-DRB1*0401, and the PRAME targeted T-cell subpopulation is primed and expanded with PRAME-derived peptides selected from Table 37 (Seq. ID. Nos. 359-368). In some embodiments, the donor cell source is HLA-DRB1*0401, and the PRAME targeted T-cell subpopulation is primed and expanded with PRAME-derived peptides comprising the peptides of Table 37 (Seq. ID. Nos. 359-368). In some embodiments, the donor cell source is HLA-DRB1*0401, and the PRAME targeted T-cell subpopulation is primed and expanded with PRAME-derived peptides comprising the peptides of Table 37 (Seq. ID. Nos. 359-368) and at least one additional set of peptides based on the donor cell source HLA-DR profile, wherein the at least one additional set of peptides are selected from the peptides of Tables 35-36 and 38-40. In some embodiments, the PRAME-derived peptides also include one or more sets of HLA-A and HLA-B restricted peptides selected from Tables 21-34 (Seq. ID Nos. 200-338).

TABLE 37
PRAME HLA-DRB1*0401 (DR4Dw4) Epitope Peptides

| SEQ ID NO. | Sequence |
|---|---|
| 359 | RRLWGSIQSRYISMS |
| 360 | RRLVELAGQSLLKDE |
| 361 | SYLIEKVKRKKNVLR |
| 362 | LGQMINLRRLLLSHI |
| 363 | EQYIAQFTSQFLSLQ |
| 364 | RGRLDQLLRHVMNPL |
| 365 | RHVMNPLETLSITNC |
| 366 | EGDVMHLSQSPSVSQ |
| 367 | LALLPSLSHCSQLTT |
| 368 | SISISALQSLLQHLI |

In some embodiments, the donor cell source is HLA-DRB1*0701, and the PRAME targeted T-cell subpopulation is primed and expanded with one or more PRAME-derived peptides selected from Table 38 (Seq. ID. Nos. 369-378). In some embodiments, the donor cell source is HLA-DRB1*0701, and the PRAME targeted T-cell subpopulation is primed and expanded with PRAME-derived peptides selected from Table 38 (Seq. ID. Nos. 369-378). In some embodiments, the donor cell source is HLA-DRB1*0701, and the PRAME targeted T-cell subpopulation is primed and expanded with PRAME-derived peptides comprising the peptides of Table 38 (Seq. ID. Nos. 369-378). In some embodiments, the donor cell source is HLA-DRB1*0701, and the PRAME targeted T-cell subpopulation is primed and expanded with PRAME-derived peptides comprising the peptides of Table 38 (Seq. ID. Nos. 369-378) and at least one additional set of peptides based on the donor cell source HLA-DR profile, wherein the at least one additional set of peptides are selected from the peptides of Tables 35-37 and 39-40. In some embodiments, the PRAME-derived peptides also include one or more sets of HLA-A and HLA-B restricted peptides selected from Tables 21-34 (Seq. ID Nos. 200-338).

TABLE 38
PRAME HLA-DRB1*0701 Epitope Peptides

| SEQ ID NO. | Sequence |
|---|---|
| 369 | RRLWGSIQSRYISMS |
| 370 | IEDLEVTCTWKLPTL |
| 371 | GDVMHLSQSPSVSQL |
| 372 | MVQLDSIEDLEVTCT |
| 373 | LSFYGNSISISALQS |
| 374 | MAAFDGRHSQTLKAM |
| 375 | EEQYIAQFTSQFLSL |
| 376 | EQYIAQFTSQFLSLQ |
| 377 | RHVMNPLETLSITNC |
| 378 | LQALLERASATLQDL |

In some embodiments, the donor cell source is HLA-DRB1*1101, and the PRAME targeted T-cell subpopulation is primed and expanded with one or more PRAME-derived peptides selected from Table 39 (Seq. ID. Nos. 379-388). In some embodiments, the donor cell source is HLA-DRB1*1101, and the PRAME targeted T-cell subpopulation is primed and expanded with PRAME-derived peptides selected from Table 39 (Seq. ID. Nos. 379-388). In some embodiments, the donor cell source is HLA-DRB1*1101, and the PRAME targeted T-cell subpopulation is primed and expanded with PRAME-derived peptides comprising the peptides of Table 39 (Seq. ID. Nos. 379-388). In some embodiments, the donor cell source is HLA-DRB1*1101, and the PRAME targeted T-cell subpopulation is primed and expanded with PRAME-derived peptides comprising the peptides of Table 39 (Seq. ID. Nos. 379-388) and at least one additional set of peptides based on the donor cell source HLA-DR profile, wherein the at least one additional set of peptides are selected from the peptides of Tables 35-38 and 40. In some embodiments, the PRAME-derived peptides also include one or more sets of HLA-A and HLA-B restricted peptides selected from Tables 21-34 (Seq. ID Nos. 200-338).

TABLE 39
PRAME HLA-DRB1*1101 Epitope Peptides

| SEQ ID NO. | Sequence |
|---|---|
| 379 | TWKLPTLAKFSPYLG |
| 380 | QSRYISMSVWTSPRR |
| 381 | AQPMTKKRKVDGLST |
| 382 | TSQFLSLQCLQALYV |
| 383 | MSVWTSPRRLVELAG |
| 384 | IAALELLPRELFPPL |
| 385 | CLPLGVLMKGQHLHL |
| 386 | QDFWTVWSGNRASLY |
| 387 | SYLIEKVKRKKNVLR |
| 388 | MQDIKMILKMVQLDS |

In some embodiments, the donor cell source is HLA-DRB1*1501, and the PRAME targeted T-cell subpopulation is primed and expanded with one or more PRAME-derived peptides selected from Table 40 (Seq. ID. Nos. 389-398). In some embodiments, the donor cell source is HLA-DRB1*1501, and the PRAME targeted T-cell subpopulation is primed and expanded with PRAME-derived peptides selected from Table 40 (Seq. ID. Nos. 389-398). In some embodiments, the donor cell source is HLA-DRB1*1501, and the PRAME targeted T-cell subpopulation is primed and expanded with PRAME-derived peptides comprising the peptides of Table 40 (Seq. ID. Nos. 389-398). In some embodiments, the donor cell source is HLA-DRB1*1501, and the PRAME targeted T-cell subpopulation is primed and expanded with PRAME-derived peptides comprising the peptides of Table 40 (Seq. ID. Nos. 389-398) and at least one additional set of peptides based on the donor cell source HLA-DR profile, wherein the at least one additional set of peptides are selected from the peptides of Tables 35-39. In some embodiments, the PRAME-derived peptides also include one or more sets of HLA-A and HLA-B restricted peptides selected from Tables 21-34 (Seq. ID Nos. 200-338).

TABLE 40
PRAME HLA-DRB1*1501 (DR2b) Epitope Peptides

| SEQ ID NO. | Sequence |
|---|---|
| 389 | HLHLETFKAVLDGLD |
| 390 | PVPLESYEDIHGTLH |
| 391 | YISMSVWTSPRRLVE |
| 392 | PLFMAAFDGRHSQTL |
| 393 | LPTLAKFSPYLGQMI |
| 394 | EQYIAQFTSQFLSLQ |
| 395 | LTTLSFYGNSISISA |
| 396 | LAKFSPYLGQMINLR |
| 397 | MERRRLWGSIQSRYI |
| 398 | GSIQSRYISMSVWTS |

Survivin Antigenic Peptides

In some embodiments, the MUSTANG composition includes survivin specific T-cells. survivin specific T-cells can be generated as described below using one or more antigenic peptides to Survivin. In some embodiments, the Survivin specific T-cells are generated using one or more antigenic peptides to Survivin, or a modified or heteroclitic peptide derived from a survivin peptide. In some embodiments, survivin specific T-cells are generated using a survivin antigen library comprising a pool of peptides (for example 15mers) containing amino acid overlap (for example 11 amino acids of overlap) between each sequence formed by scanning the protein amino acid sequence SEQ. ID. No. 399 (UniProt KB-015392) for human baculoviral inhibitor of apoptosis repeat-containing 5 (Survivin):

MGAPTLPPAWQPFLKDHRISTFKNWPFLEGCACTPERMAEAGFIHCPTE
NEPDLQCFFCFKELEGWEPDDDPIEEHKKHSSGCAFLSVKKQFEELTLG
EFLKLDRERAKNKIAKETNNKKKEFEETAKKVRRAIEQLAAMD

Overlapping antigenic libraries are commercially available, for example, from JPT, for example, from JPT (Product Code: PM-Survivin (Pep Mix™ Human (Survivin)). In some embodiments, the survivin specific T-cells are generated using a commercially available overlapping antigenic library made up of survivin peptides.

In some embodiments, the survivin specific T-cells are generated using one or more antigenic peptides to survivin, or a modified or heteroclitic peptide derived from a Survivin peptide,

In some embodiments, the survivin specific T-cells are generated with peptides that recognize class I MHC molecules. In some embodiments, the survivin specific T-cells are generated with peptides that recognize class II MEW molecules. In some embodiments, the Survivin specific T-cells are generated with peptides that recognize both class I and class II MEW molecules.

In some embodiments, the survivin peptides used to prime and expand a T-cell subpopulation includes specifically selected HLA-restricted peptides generated by determining the HLA profile of the donor source, and including peptides derived from survivin that best match the donor's HLA. In some embodiments, the survivin peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting survivin derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 41-47, the HLA-B peptides are selected from the peptides of Tables 48-54, and the HLA-DR peptides are selected from the peptides of Tables 55-60. For example, if the donor cell source has an HLA profile that is HLA-A*01/*02:01; HLA-B*15:01/*18; and HLA-DRB1*0101/*0301, then the survivin peptides used to prime and expand the survivin specific T-cell subpopulation are restricted to the specific HLA profile, and may include the peptides identified in Table 41 (Seq. ID. Nos. 400-409) for HLA-A*01; Table 42 (Seq. ID. No. 410-419) for HLA-A*02:01; Table 50 (Seq. ID. No. 490-500) for HLA-B*15:01; Table 51 (Seq. ID. No. 501-510) for HLA-B*18; Table 55 (Seq. ID. No. 541-550) for HLA-DRB1*0101; and Table 56 (Seq. ID. No. 551-560) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the survivin targeted T-cell subpopulation is primed and expanded with one or more survivin-derived peptides selected from Table 41 (Seq. ID. Nos. 400-409). In some embodiments, the donor cell source is HLA-A*01, and the survivin targeted T-cell subpopulation is primed and expanded with survivin-derived peptides selected from Table 41 (Seq. ID. Nos. 400-409). In some embodiments, the donor cell source is HLA-A*01, and the survivin targeted T-cell subpopulation is primed and expanded with survivin-derived peptides comprising the peptides of Table 41 (Seq. ID. Nos. 400-409). In some embodiments, the donor cell source is HLA-A*01, and the survivin targeted T-cell subpopulation is primed and expanded with survivin-derived peptides comprising the peptides of Table 41 (Seq. ID. Nos. 400-409) and at least one additional set of peptides based on the donor cell source HLA-A profile, wherein the at least one additional set of peptides are selected from the peptides of Tables 42-47. In some embodiments, the survivin-derived peptides also include one or more sets of HLA-B and HLA-DR restricted peptides selected from Tables 48-60 (Seq. ID Nos. 470-600).

TABLE 41
Survivin HLA-A*01 Epitope Peptides

| SEQ ID NO. | Sequence |
|---|---|
| 400 | PTENEPDLAQC |
| 401 | KLDRERAKNKI |
| 402 | LKDHRISTFKN |
| 403 | STFKNWPFLEG |
| 404 | DDDPIEEHKKH |
| 405 | PTENEPDLAQ |
| 406 | PTENEPDLA |
| 407 | LTLGEFLKL |
| 408 | LGEFLKLDR |
| 409 | KLDRERAKN |

In some embodiments, the donor cell source is HLA-A*02:01, and the survivin targeted T-cell subpopulation is primed and expanded with one or more survivin-derived peptides selected from Table 42 (Seq. ID. Nos. 410-419). In some embodiments, the donor cell source is HLA-A*02:01, and the survivin targeted T-cell subpopulation is primed and expanded with survivin-derived peptides selected from Table 42 (Seq. ID. Nos. 410-419). In some embodiments, the donor cell source is HLA-A*02:01, and the survivin targeted T-cell subpopulation is primed and expanded with survivin-derived peptides comprising the peptides of Table 42 (Seq. ID. Nos. 410-419). In some embodiments, the donor cell source is HLA-A*02:01, and the survivin targeted T-cell subpopulation is primed and expanded with survivin-derived peptides comprising the peptides of Table 42 (Seq. ID. Nos. 410-419) and at least one additional set of peptides based on the donor cell source HLA-A profile, wherein the at least one additional set of peptides are selected from the peptides of Tables 41, and 43-47. In some embodiments, the survivin-derived peptides also include one or more sets of HLA-B and HLA-DR restricted peptides selected from Tables 48-60 (Seq. ID Nos. 470-600).

TABLE 42
Survivin HLA-A*02:01 Epitope Peptides

| SEQ ID NO. | Sequence |
|---|---|
| 410 | TLPPAWQPFL |
| 411 | ELTLGEFLKL |
| 412 | FLKDHRISTF |
| 413 | LTLGEFLKL |
| 414 | KVRRAIEQL |
| 415 | RAIEQLAAM |
| 416 | STFKNWPFL |
| 417 | FLKDHRIST |
| 418 | SVKKQFEEL |
| 419 | TLGEFLKLD |

In some embodiments, the donor cell source is HLA-A*03, and the survivin targeted T-cell subpopulation is primed and expanded with one or more survivin-derived peptides selected from Table 43 (Seq. ID. Nos. 420-429). In some embodiments, the donor cell source is HLA-A*03, and the survivin targeted T-cell subpopulation is primed and expanded with survivin-derived peptides selected from Table 43 (Seq. ID. Nos. 420-429). In some embodiments, the donor cell source is HLA-A*03, and the survivin targeted T-cell subpopulation is primed and expanded with survivin-derived peptides comprising the peptides of Table 43 (Seq. ID. Nos. 420-429). In some embodiments, the donor cell source is HLA-A*03, and the survivin targeted T-cell subpopulation is primed and expanded with survivin-derived peptides comprising the peptides of Table 43 (Seq. ID. Nos. 420-429) and at least one additional set of peptides based on the donor cell source HLA-A profile, wherein the at least one additional set of peptides are selected from the peptides of Tables 41-42 and 44-47. In some embodiments, the survivin-derived peptides also include one or more sets of HLA-B and HLA-DR restricted peptides selected from Tables 48-60 (Seq. ID Nos. 470-600).

TABLE 43
Survivin HLA-A*03 Epitope Peptides

| SEQ ID NO. | Sequence |
|---|---|
| 420 | KLDRERAKNK |
| 421 | FLKDHRISTF |
| 422 | FLKLDRERAK |
| 423 | KIAKETNNKK |
| 424 | DLAQCFFCFK |
| 425 | ELTLGEFLK |
| 426 | KIAKETNNK |
| 427 | KVRRAIEQL |
| 428 | SGCAFLSVK |
| 429 | KLDRERAKN |

In some embodiments, the donor cell source is HLA-A*11:01, and the survivin targeted T-cell subpopulation is primed and expanded with one or more survivin-derived peptides selected from Table 44 (Seq. ID. Nos. 430-439). In some embodiments, the donor cell source is HLA-A*11:01, and the survivin targeted T-cell subpopulation is primed and expanded with survivin-derived peptides selected from Table 44 (Seq. ID. Nos. 430-439). In some embodiments, the donor cell source is HLA-A*11:01, and the survivin targeted T-cell subpopulation is primed and expanded with survivin-derived peptides comprising the peptides of Table 44 (Seq. ID. Nos. 430-439). In some embodiments, the donor cell source is HLA-A*11:01, and the survivin targeted T-cell subpopulation is primed and expanded with survivin-derived peptides comprising the peptides of Table 44 (Seq. ID. Nos. 430-439), and at least one additional set of peptides based on the donor cell source HLA-A profile, wherein the at least one additional set of peptides are selected from the peptides of Tables 41-43 and 45-47. In some embodiments, the survivin-derived peptides also include one or more sets of HLA-B and HLA-DR restricted peptides selected from Tables 48-60 (Seq. ID Nos. 470-600).

TABLE 44
Survivin HLA-A*11:01 Epitope Peptides

| SEQ ID NO. | Sequence |
|---|---|
| 430 | SSGCAFLSVK |
| 431 | DLAQCFFCFK |
| 432 | SGCAFLSVKK |
| 433 | TLGEFLKLDR |
| 434 | STFKNWPFLE |
| 435 | KLDRERAKNK |
| 436 | KIAKETNNKK |
| 437 | SSGCAFLSV |
| 438 | GCAFLSVKK |
| 439 | ELTLGEFLK |

In some embodiments, the donor cell source is HLA-A*24:02, and the survivin targeted T-cell subpopulation is primed and expanded with one or more survivin-derived peptides selected from Table 45 (Seq. ID. Nos. 440-449). In some embodiments, the donor cell source is HLA-A*24:02, and the survivin targeted T-cell subpopulation is primed and expanded with survivin-derived peptides selected from Table 45 (Seq. ID. Nos. 440-449). In some embodiments, the donor cell source is HLA-A*24:02, and the survivin targeted T-cell subpopulation is primed and expanded with survivin-derived peptides comprising the peptides of Table 45 (Seq. ID. Nos. 440-449). In some embodiments, the donor cell source is HLA-A*24:02, and the survivin targeted T-cell subpopulation is primed and expanded with survivin-derived peptides comprising the peptides of Table 45 (Seq. ID. Nos. 440-449), and at least one additional set of peptides based on the donor cell source HLA-A profile, wherein the at least one additional set of peptides are selected from the peptides of Tables 41-44 and 46-47. In some embodiments, the survivin-derived peptides also include one or more sets of HLA-B and HLA-DR restricted peptides selected from Tables 48-60 (Seq. ID Nos. 470-600).

TABLE 45
Survivin HLA-A24:02 Epitope Peptides

| SEQ ID NO. | Sequence |
|---|---|
| 440 | QFEELTLGEF |
| 441 | TLPPAWQPFL |
| 442 | PDLAQCFFCF |
| 443 | PTLPPAWQPF |
| 444 | NEPDLAQCFF |
| 445 | LSVKKQFEEL |
| 446 | ELTLGEFLKL |
| 447 | AFLSVKKQF |
| 448 | LTLGEFLKL |
| 449 | TLPPAWQPF |

In some embodiments, the donor cell source is HLA-A*26, and the survivin targeted T-cell subpopulation is primed and expanded with one or more survivin-derived peptides selected from Table 46 (Seq. ID. Nos. 450-459). In some embodiments, the donor cell source is HLA-A*26, and the survivin targeted T-cell subpopulation is primed and expanded with survivin-derived peptides selected from Table 46 (Seq. ID. Nos. 450-459). In some embodiments, the donor cell source is HLA-A*26, and the survivin targeted T-cell subpopulation is primed and expanded with survivin-derived peptides comprising the peptides of Table 46 (Seq. ID. Nos. 450-459). In some embodiments, the donor cell source is HLA-A*26, and the survivin targeted T-cell subpopulation is primed and expanded with survivin-derived peptides comprising the peptides of Table 46 (Seq. ID. Nos. 450-459) and at least one additional set of peptides based on the donor cell source HLA-A profile, wherein the at least one additional set of peptides are selected from the peptides of Tables 41-45 and 47. In some embodiments, the survivin-derived peptides also include one or more sets of HLA-B and HLA-DR restricted peptides selected from Tables 48-60 (Seq. ID Nos. 470-600).

TABLE 46
Survivin HLA-A*26 Epitope Peptides

| SEQ ID NO. | Sequence |
|---|---|
| 450 | ELTLGEFLKL |
| 451 | ENEPDLAQCF |
| 452 | ETAKKVRRAI |
| 453 | ETNNKKKEFE |
| 454 | ETNNKKKEF |
| 455 | ETAKKVRRA |
| 456 | KVRRAIEQL |
| 457 | STFKNWPFL |
| 458 | EELTLGEFL |
| 459 | SVKKQFEEL |

In some embodiments, the donor cell source is HLA-A*68:01, and the survivin targeted T-cell subpopulation is primed and expanded with one or more survivin-derived peptides selected from Table 47 (Seq. ID. Nos. 460-469). In some embodiments, the donor cell source is HLA-A*68:01, and the survivin targeted T-cell subpopulation is primed and expanded with survivin-derived peptides selected from Table 47 (Seq. ID. Nos. 460-469). In some embodiments, the donor cell source is HLA-A*68:01, and the survivin targeted T-cell subpopulation is primed and expanded with survivin-derived peptides comprising the peptides of Table 47 (Seq. ID. Nos. 460-469). In some embodiments, the donor cell source is HLA-A*68:01, and the survivin targeted T-cell subpopulation is primed and expanded with survivin-derived peptides comprising the peptides of Table 47 (Seq. ID. Nos. 460-469), and at least one additional set of peptides based on the donor cell source HLA-A profile, wherein the at least one additional set of peptides are selected from the peptides of Tables 41-46. In some embodiments, the survivin-derived peptides also include one or more sets of HLA-B and HLA-DR restricted peptides selected from Tables 48-60 (Seq. ID Nos. 470-600).

TABLE 47
Survivin HLA-A*68:01 Epitope Peptides

| SEQ ID NO. | Sequence |
|---|---|
| 460 | LTLGEFLKLDR |
| 461 | PAWQPFLKDHR |
| 462 | SSGCAFLSVKK |
| 463 | EFEETAKKVRR |
| 464 | ETAKKVRRAIE |
| 465 | DLAQCFFCFK |
| 466 | EETAKKVRR |
| 467 | ERAKNKIAK |
| 468 | ETAKKVRRA |
| 469 | ELTLGEFLK |

In some embodiments, the donor cell source is HLA-B*07:02, and the survivin targeted T-cell subpopulation is primed and expanded with one or more survivin-derived peptides selected from Table 48 (Seq. ID. Nos. 470-479). In some embodiments, the donor cell source is HLA-B*07:02, and the survivin targeted T-cell subpopulation is primed and expanded with survivin-derived peptides selected from Table 48 (Seq. ID. Nos. 470-479). In some embodiments, the donor cell source is HLA-B*07:02, and the survivin targeted T-cell subpopulation is primed and expanded with survivin-derived peptides comprising the peptides of Table 48 (Seq. ID. Nos. 470-479). In some embodiments, the donor cell source is HLA-B*07:02, and the survivin targeted T-cell subpopulation is primed and expanded with survivin-derived peptides comprising the peptides of Table 48 (Seq. ID. Nos. 470-479), and at least one additional set of peptides based on the donor cell source HLA-B profile, wherein the at least one additional set of peptides are selected from the peptides of Tables 49-54. In some embodiments, the survivin-derived peptides also include one or more sets of HLA-A and HLA-DR restricted peptides selected from Tables 41-47 and 55-60 (Seq. ID Nos. 400-469 and 541-600).

TABLE 48
Survivin HLA-B*07:02 Epitope Peptides

| SEQ ID NO. | Sequence |
|---|---|
| 470 | LPPAWQPFL |
| 471 | CPTENEPDL |
| 472 | EPDLAQCFF |
| 473 | APTLPPAWQ |
| 474 | QPFLKDHRI |
| 475 | KHSSGCAFL |
| 476 | LTLGEFLKL |
| 477 | WPFLEGCACT |
| 478 | TPERMAEAGF |
| 479 | CPTENEPDLA |

In some embodiments, the donor cell source is HLA-B*08, and the survivin targeted T-cell subpopulation is primed and expanded with one or more survivin-derived peptides selected from Table 49 (Seq. ID. Nos. 480-489). In some embodiments, the donor cell source is HLA-B*08, and the survivin targeted T-cell subpopulation is primed and expanded with survivin-derived peptides selected from Table 49 (Seq. ID. Nos. 480-489). In some embodiments, the donor cell source is HLA-B*08, and the survivin targeted T-cell subpopulation is primed and expanded with survivin-derived peptides comprising the peptides of Table 49 (Seq. ID. Nos. 480-489). In some embodiments, the donor cell source is HLA-B*08, and the survivin targeted T-cell subpopulation is primed and expanded with survivin-derived peptides comprising the peptides of Table 49 (Seq. ID. Nos. 480-489) and at least one additional set of peptides based on the donor cell source HLA-B profile, wherein the at least one additional set of peptides are selected from the peptides of Tables 48 and 50-54. In some embodiments, the survivin-derived peptides also include one or more sets of HLA-A and HLA-DR restricted peptides selected from Tables 41-47 and 55-60 (Seq. ID Nos. 400-469 and 541-600).

TABLE 49
Survivin HLA-B*08 Epitope Peptides

| SEQ ID NO. | Sequence |
|---|---|
| 480 | RAKNKIAKE |
| 481 | QPFLKDHRI |
| 482 | SVKKQFEEL |
| 483 | NNKKKEFEE |
| 484 | TAKKVRRAI |
| 485 | AKKVRRAI |
| 486 | FLSVKKQF |
| 487 | RAKNKIAK |
| 488 | RERAKNKI |
| 489 | VKKQFEEL |

In some embodiments, the donor cell source is HLA-B*15:01, and the survivin targeted T-cell subpopulation is primed and expanded with one or more survivin-derived peptides selected from Table 50 (Seq. ID. Nos. 490-500). In some embodiments, the donor cell source is HLA-B*15:01, and the survivin targeted T-cell subpopulation is primed and expanded with survivin-derived peptides selected from Table 50 (Seq. ID. Nos. 490-500). In some embodiments, the donor cell source is HLA-B*15:01, and the survivin targeted T-cell subpopulation is primed and expanded with survivin-derived peptides comprising the peptides of Table 50 (Seq. ID. Nos. 490-500). In some embodiments, the donor cell source is HLA-B*15:01, and the survivin targeted T-cell subpopulation is primed and expanded with survivin-derived peptides comprising the peptides of Table 50 (Seq. ID. Nos. 490-500) and at least one additional set of peptides based on the donor cell source HLA-B profile, wherein the at least one additional set of peptides are selected from the peptides of Tables 48-49 and 51-54. In some embodiments, the survivin-derived peptides also include one or more sets of HLA-A and HLA-DR restricted peptides selected from Tables 41-47 and 55-60 (Seq. ID Nos. 400-469 and 541-600).

TABLE 50
Survivin HLA-B*15:01 (B62) Epitope Peptides

| SEQ ID NO. | Sequence |
|---|---|
| 490 | FLKDHRISTF |
| 491 | KQFEELTLGE |
| 492 | TLPPAWQPFL |
| 493 | ELEGWEPDDD |
| 495 | TLGEFLKLDR |
| 496 | TLPPAWQPF |
| 497 | DLAQCFFCF |
| 498 | KQFEELTLG |
| 499 | FLKDHRIST |
| 500 | KVRRAIEQL |

In some embodiments, the donor cell source is HLA-B*18, and the survivin targeted T-cell subpopulation is primed and expanded with one or more survivin-derived peptides selected from Table 51 (Seq. ID. Nos. 501-510). In some embodiments, the donor cell source is HLA-B*18, and the survivin targeted T-cell subpopulation is primed and expanded with survivin-derived peptides selected from Table 51 (Seq. ID. Nos. 501-510). In some embodiments, the donor cell source is HLA-B*18, and the survivin targeted T-cell subpopulation is primed and expanded with survivin-derived peptides comprising the peptides of Table 51 (Seq. ID. Nos. 501-510). In some embodiments, the donor cell source is HLA-B*18, and the survivin targeted T-cell subpopulation is primed and expanded with survivin-derived peptides comprising the peptides of Table 51 (Seq. ID. Nos. 501-510) and at least one additional set of peptides based on the donor cell source HLA-B profile, wherein the at least one additional set of peptides are selected from the peptides of Tables 48-50 and 52-54. In some embodiments, the survivin-derived peptides also include one or more sets of HLA-A and HLA-DR restricted peptides selected from Tables 41-47 and 55-60 (Seq. ID Nos. 400-469 and 541-600).

TABLE 51
Survivin HLA-B*18 Epitope Peptides

| SEQ ID NO. | Sequence |
|---|---|
| 501 | EELTLGEFL |
| 502 | FEELTLGEF |
| 503 | NEPDLAQCF |
| 504 | PERMAEAGF |
| 505 | DLAQCFFCF |
| 506 | KELEGWEPD |
| 507 | EELTLGEF |
| 508 | EEHKKHSS |
| 509 | KELEGWEP |
| 510 | KQFEELTL |

In some embodiments, the donor cell source is HLA-B*27:05, and the survivin targeted T-cell subpopulation is primed and expanded with one or more survivin-derived peptides selected from Table 52 (Seq. ID. Nos. 511-520). In some embodiments, the donor cell source is HLA-B*27:05, and the survivin targeted T-cell subpopulation is primed and expanded with survivin-derived peptides selected from Table 52 (Seq. ID. Nos. 511-520). In some embodiments, the donor cell source is HLA-B*27:05, and the survivin targeted T-cell subpopulation is primed and expanded with survivin-derived peptides comprising the peptides of Table 52 (Seq. ID. Nos. 511-520). In some embodiments, the donor cell source is HLA-B*27:05, and the survivin targeted T-cell subpopulation is primed and expanded with survivin-derived peptides comprising the peptides of Table 52 (Seq. ID. Nos. 511-520) and at least one additional set of peptides based on the donor cell source HLA-B profile, wherein the at least one additional set of peptides are selected from the peptides of Tables 48-51 and 53-54. In some embodiments, the survivin-derived peptides also include one or more sets of HLA-A and HLA-DR restricted peptides selected from Tables 41-47 and 55-60 (Seq. ID Nos. 400-469 and 541-600).

TABLE 52
Survivin HLA-B*27:05 Epitope Peptides

| SEQ ID NO. | Sequence |
|---|---|
| 511 | RRAIEQLAAM |
| 512 | GEFLKLDRER |
| 513 | ERMAEAGFIH |
| 514 | ERAKNKIAKE |
| 515 | KIAKETNNKK |
| 516 | ERAKNKIAK |
| 517 | DRERAKNKI |
| 518 | KEFEETAKK |
| 519 | ERMAEAGFI |
| 520 | GCAFLSVKK |

In some embodiments, the donor cell source is HLA-B*35:01, and the survivin targeted T-cell subpopulation is primed and expanded with one or more survivin-derived peptides selected from Table 53 (Seq. ID. Nos. 521-530). In some embodiments, the donor cell source is HLA-B*35:01, and the survivin targeted T-cell subpopulation is primed and expanded with survivin-derived peptides selected from Table 53 (Seq. ID. Nos. 521-530). In some embodiments, the donor cell source is HLA-B*35:01, and the survivin targeted T-cell subpopulation is primed and expanded with survivin-derived peptides comprising the peptides of Table 53 (Seq. ID. Nos. 521-530). In some embodiments, the donor cell source is HLA-B*35:01, and the survivin targeted T-cell subpopulation is primed and expanded with survivin-derived peptides comprising the peptides of Table 53 (Seq. ID. Nos. 521-530) and at least one additional set of peptides based on the donor cell source HLA-B profile, wherein the at least one additional set of peptides are selected from the peptides of Tables 48-52 and 54. In some embodiments, the survivin-derived peptides also include one or more sets of HLA-A and HLA-DR restricted peptides selected from Tables 41-47 and 55-60 (Seq. ID Nos. 400-469 and 541-600).

TABLE 53
Survivin HLA-B*35:01 Epitope Peptides

| SEQ ID NO. | Sequence |
|---|---|
| 521 | TPERMAEAGF |
| 522 | LPPAWQPFLK |
| 523 | EPDDDPIEEH |
| 524 | LSVKKQFEEL |
| 525 | LPPAWQPFL |
| 526 | CPTENEPDL |
| 527 | EPDLAQCFF |
| 528 | QPFLKDHRI |
| 529 | TPERMAEAG |
| 530 | EPDDDPIEE |

In some embodiments, the donor cell source is HLA-B*58:02, and the survivin targeted T-cell subpopulation is primed and expanded with one or more survivin-derived peptides selected from Table 54 (Seq. ID. Nos. 531-540). In some embodiments, the donor cell source is HLA-B*58:02, and the survivin targeted T-cell subpopulation is primed and expanded with survivin-derived peptides selected from Table 54 (Seq. ID. Nos. 531-540). In some embodiments, the donor cell source is HLA-B*58:02, and the survivin targeted T-cell subpopulation is primed and expanded with survivin-derived peptides comprising the peptides of Table 54 (Seq. ID. Nos. 531-540). In some embodiments, the donor cell source is HLA-B*58:02, and the survivin targeted T-cell subpopulation is primed and expanded with survivin-derived peptides comprising the peptides of Table 54 (Seq. ID. Nos. 531-540) and at least one additional set of peptides based on the donor cell source HLA-B profile, wherein the at least one additional set of peptides are selected from the peptides of Tables 48-53. In some embodiments, the survivin-derived peptides also include one or more sets of HLA-A and HLA-DR restricted peptides selected from Tables 41-47 and 55-60 (Seq. ID Nos. 400-469 and 541-600).

TABLE 54
Survivin HLA-B*58:02 Epitope Peptides

| SEQ ID NO. | Sequence |
|---|---|
| 531 | ETAKKVRRAI |
| 532 | PTLPPAWQPF |
| 533 | ISTFKNWPFL |
| 534 | LSVKKQFEEL |
| 535 | TAKKVRRAI |
| 536 | RAIEQLAAM |
| 537 | KVRRAIEQL |
| 538 | ISTFKNWPF |
| 539 | LTLGEFLKL |
| 540 | GAPTLPPAW |

In some embodiments, the donor cell source is HLA-DRB1*0101, and the survivin targeted T-cell subpopulation is primed and expanded with one or more survivin-derived peptides selected from Table 55 (Seq. ID. Nos. 541-550). In some embodiments, the donor cell source is HLA-DRB1*0101, and the survivin targeted T-cell subpopulation is primed and expanded with survivin-derived peptides selected from Table 55 (Seq. ID. Nos. 541-550). In some embodiments, the donor cell source is HLA-DRB1*0101, and the survivin targeted T-cell subpopulation is primed and expanded with survivin-derived peptides comprising the peptides of Table 55 (Seq. ID. Nos. 541-550). In some embodiments, the donor cell source is HLA-DRB1*0101, and the survivin targeted T-cell subpopulation is primed and expanded with survivin-derived peptides comprising the peptides of Table 55 (Seq. ID. Nos. 541-550) and at least one additional set of peptides based on the donor cell source HLA-DR profile, wherein the at least one additional set of peptides are selected from the peptides of Tables 56-60. In some embodiments, the survivin-derived peptides also include one or more sets of HLA-A and HLA-B restricted peptides selected from Tables 41-54 (Seq. ID Nos. 400-540).

TABLE 55
Survivin HLA-DRB1*0101 Epitope Peptides

| SEQ ID NO. | Sequence |
|---|---|
| 541 | FFCFKELEGWEPDDD |
| 542 | FKNWPFLEGCACTPE |
| 543 | LGEFLKLDRERAKNK |
| 544 | NWPFLEGCACTPERM |
| 545 | KKQFEELTLGEFLKL |
| 546 | CTPERMAEAGFIHCP |
| 547 | FEELTLGEFLKLDRE |
| 548 | MGAPTLPPAWQPFLK |
| 549 | KKKEFEETAKKVRRA |
| 550 | AKKVRRAIEQLAAMD |

In some embodiments, the donor cell source is HLA-DRB1*0301, and the survivin targeted T-cell subpopulation is primed and expanded with one or more survivin-derived peptides selected from Table 56 (Seq. ID. Nos. 551-560). In some embodiments, the donor cell source is HLA-DRB1*0301, and the survivin targeted T-cell subpopulation is primed and expanded with survivin-derived peptides selected from Table 56 (Seq. ID. Nos. 551-560). In some embodiments, the donor cell source is HLA-DRB1*0301, and the survivin targeted T-cell subpopulation is primed and expanded with survivin-derived peptides comprising the peptides of Table 56 (Seq. ID. Nos. 551-560). In some embodiments, the donor cell source is HLA-DRB1*0301, and the survivin targeted T-cell subpopulation is primed and expanded with survivin-derived peptides comprising the peptides of Table 56 (Seq. ID. Nos. 551-560) and at least one additional set of peptides based on the donor cell source HLA-DR profile, wherein the at least one additional set of peptides are selected from the peptides of Tables 55 and 57-60. In some embodiments, the survivin-derived peptides also include one or more sets of HLA-A and HLA-B restricted peptides selected from Tables 41-54 (Seq. ID Nos. 400-540).

TABLE 56
Survivin HLA-DRB1*0301 (DR17) Epitope Peptides

| SEQ ID NO. | Sequence |
|---|---|
| 551 | GEFLKLDRERAKNKI |
| 552 | WQPFLKDHRISTFKN |
| 553 | APTLPPAWQPFLKDH |
| 554 | DHRISTFKNWPFLEG |
| 555 | FEELTLGEFLKLDRE |
| 556 | PTENEPDLAQCFFCF |
| 557 | QPFLKDHRISTFKNW |
| 558 | GCAFLSVKKQFEELT |
| 559 | ELTLGEFLKLDRERA |
| 560 | AKKVRRAIEQLAAMD |

In some embodiments, the donor cell source is HLA-DRB1*0401, and the survivin targeted T-cell subpopulation is primed and expanded with one or more survivin-derived peptides selected from Table 57 (Seq. ID. Nos. 561-570). In some embodiments, the donor cell source is HLA-DRB1*0401, and the survivin targeted T-cell subpopulation is primed and expanded with survivin-derived peptides selected from Table 57 (Seq. ID. Nos. 561-570). In some embodiments, the donor cell source is HLA-DRB1*0401, and the survivin targeted T-cell subpopulation is primed and expanded with survivin-derived peptides comprising the peptides of Table 57 (Seq. ID. Nos. 561-570). In some embodiments, the donor cell source is HLA-DRB1*0401, and the survivin targeted T-cell subpopulation is primed and expanded with survivin-derived peptides comprising the peptides of Table 57 (Seq. ID. Nos. 561-570) and at least one additional set of peptides based on the donor cell source HLA-DR profile, wherein the at least one additional set of peptides are selected from the peptides of Tables 55-56 and 58-60. In some embodiments, the survivin-derived peptides also include one or more sets of HLA-A and HLA-B restricted peptides selected from Tables 41-54 (Seq. ID Nos. 400-540).

TABLE 57
Survivin HLA-DRB1*0401 (DR4Dw4) Epitope Peptides

| SEQ ID NO. | Sequence |
|---|---|
| 561 | WQPFLKDHRISTFKN |
| 562 | LGEFLKLDRERAKNK |
| 563 | APTLPPAWQPFLKDH |
| 564 | KNKIAKETNNKKKEF |
| 565 | DHRISTFKNWPFLEG |
| 566 | GEFLKLDRERAKNKI |
| 567 | FLKLDRERAKNKIAK |
| 568 | AKKVRRAIEQLAAMD |
| 569 | FLKDHRISTFKNWPF |
| 570 | RMAEAGFIHCPTENE |

In some embodiments, the donor cell source is HLA-DRB1*0701, and the survivin targeted T-cell subpopulation is primed and expanded with one or more survivin-derived peptides selected from Table 58 (Seq. ID. Nos. 571-580). In some embodiments, the donor cell source is HLA-DRB1*0701, and the survivin targeted T-cell subpopulation is primed and expanded with survivin-derived peptides selected from Table 58 (Seq. ID. Nos. 571-580). In some embodiments, the donor cell source is HLA-DRB1*0701, and the survivin targeted T-cell subpopulation is primed and expanded with survivin-derived peptides comprising the peptides of Table 58 (Seq. ID. Nos. 571-580). In some embodiments, the donor cell source is HLA-DRB1*0701, and the survivin targeted T-cell subpopulation is primed and expanded with survivin-derived peptides comprising the peptides of Table 58 (Seq. ID. Nos. 571-580) and at least one additional set of peptides based on the donor cell source HLA-DR profile, wherein the at least one additional set of peptides are selected from the peptides of Tables 55-57 and 59-60. In some embodiments, the survivin-derived peptides also include one or more sets of HLA-A and HLA-B restricted peptides selected from Tables 41-54 (Seq. ID Nos. 400-540).

TABLE 58
Survivin HLA-DRB1*0701 Epitope Peptides

| SEQ ID NO. | Sequence |
|---|---|
| 571 | AKKVRRAIEQLAAMD |
| 572 | APTLPPAWQPFLKDH |
| 573 | DHRISTFKNWPFLEG |
| 574 | LEGCACTPERMAEAG |
| 575 | EAGFIHCPTENEPDL |
| 576 | KKEFEETAKKVRRAI |
| 577 | AQCFFCFKELEGWEP |
| 578 | QCFFCFKELEGWEPD |
| 579 | LEGWEPDDDPIEEHK |
| 580 | KKQFEELTLGEFLKL |

In some embodiments, the donor cell source is HLA-DRB1*1101, and the survivin targeted T-cell subpopulation is primed and expanded with one or more survivin-derived peptides selected from Table 59 (Seq. ID. Nos. 581-590). In some embodiments, the donor cell source is HLA-DRB1*1101, and the survivin targeted T-cell subpopulation is primed and expanded with survivin-derived peptides selected from Table 59 (Seq. ID. Nos. 581-590). In some embodiments, the donor cell source is HLA-DRB1*1101, and the survivin targeted T-cell subpopulation is primed and expanded with survivin-derived peptides comprising the peptides of Table 59 (Seq. ID. Nos. 581-590). In some embodiments, the donor cell source is HLA-DRB1*1101, and the survivin targeted T-cell subpopulation is primed and expanded with survivin-derived peptides comprising the peptides of Table 59 (Seq. ID. Nos. 581-590) and at least one additional set of peptides based on the donor cell source HLA-DR profile, wherein the at least one additional set of peptides are selected from the peptides of Tables 55-58 and 60. In some embodiments, the survivin-derived peptides also include one or more sets of HLA-A and HLA-B restricted peptides selected from Tables 41-54 (Seq. ID Nos. 400-540).

TABLE 59
Survivin HLA-DRB1*1101 Epitope Peptides

| SEQ ID NO. | Sequence |
|---|---|
| 581 | LGEFLKLDRERAKNK |
| 582 | GCAFLSVKKQFEELT |
| 583 | FFCFKELEGWEPDDD |
| 584 | DDPIEEHKKHSSGCA |
| 585 | KKEFEETAKKVRRAI |
| 586 | PPAWQPFLKDHRIST |
| 587 | WQPFLKDHRISTFKN |
| 588 | AWQPFLKDHRISTFK |
| 589 | AQCFFCFKELEGWEP |
| 590 | ISTFKNWPFLEGCAC |

In some embodiments, the donor cell source is HLA-DRB1*1501, and the survivin targeted T-cell subpopulation is primed and expanded with one or more survivin-derived peptides selected from Table 60 (Seq. ID. Nos. 591-600). In some embodiments, the donor cell source is HLA-DRB1*1501, and the survivin targeted T-cell subpopulation is primed and expanded with survivin-derived peptides selected from Table 60 (Seq. ID. Nos. 591-600). In some embodiments, the donor cell source is HLA-DRB1*1501, and the survivin targeted T-cell subpopulation is primed and expanded with survivin-derived peptides comprising the peptides of Table 60 (Seq. ID. Nos. 591-600). In some embodiments, the donor cell source is HLA-DRB1*1501, and the survivin targeted T-cell subpopulation is primed and expanded with survivin-derived peptides comprising the peptides of Table 60 (Seq. ID. Nos. 591-600) and at least one additional set of peptides based on the donor cell source HLA-DR profile, wherein the at least one additional set of peptides are selected from the peptides of Tables 55-59. In some embodiments, the survivin-derived peptides also include one or more sets of HLA-A and HLA-B restricted peptides selected from Tables 41-54 (Seq. ID Nos. 400-540).

TABLE 60
Survivin HLA-DRB1*1501 (DR2b) Epitope Peptides

| SEQ ID NO. | Sequence |
|---|---|
| 591 | LGEFLKLDRERAKNK |
| 592 | GCAFLSVKKQFEELT |
| 593 | FFCFKELEGWEPDDD |
| 594 | DDPIEEHKKHSSGCA |
| 595 | KKEFEETAKKVRRAI |
| 596 | PPAWQPFLKDHRIST |
| 597 | WQPFLKDHRISTFKN |
| 598 | AWQPFLKDHRISTFK |
| 599 | AQCFFCFKELEGWEP |
| 600 | ISTFKNWPFLEGCAC |

NY-ESO-1 Antigenic Peptides

In some embodiments, the MUSTANG composition includes NY-ESO-1 (cancer/testis antigen 1) specific T-cells. NY-ESO-1 specific T-cells can be generated as described below using one or more antigenic peptides to NY-ESO-1. In some embodiments, the NY-ESO-1 specific T-cells are generated using one or more antigenic peptides to NY-ESO-1, or a modified or heteroclitic peptide derived from a NY-ESO-1 peptide. In some embodiments, NY-ESO-1 specific T-cells are generated using a NY-ESO-1 antigen library comprising a pool of peptides (for example 15mers) containing amino acid overlap (for example 11 amino acids of overlap) between each sequence formed by scanning the protein amino acid sequence SEQ. ID. No. 601 (UniProt KB-P78358) for NY-ESO-1:

MQAEGRGTGGSTGDADGPGGPGIPDGPGGNAGGPGEAGATGGRGPRGAG
AARASGPGGGAPRGPHGGAASGLNGCCRCGARGPESRLLEFYLAMPFAT
PMEAELARRSLAQDAPPLPVPGVLLKEFTVSGNILTIRLTAADHRQLQL
SISSCLQQLSLLMWITQCFLPVFLAQPPSGQRR.

Overlapping antigenic libraries are commercially available, for example, from JPT, for example, from JPT (Product Code: PM-NYE (Pep Mix™ Human (NY-ESO-1)). In some embodiments, the NY-ESO-1 specific T-cells are generated using a commercially available overlapping antigenic library made up of NY-ESO-1 peptides.

In some embodiments, the NY-ESO-1 specific T-cells are generated using one or more antigenic peptides to NY-ESO-1, or a modified or heteroclitic peptide derived from a NY-ESO-1 peptide. In some embodiments, the NY-ESO-1 specific T-cells are generated with peptides that recognize class I MHC molecules. In some embodiments, the NY-ESO-1 specific T-cells are generated with peptides that recognize class II MHC molecules. In some embodiments, the NY-ESO-1 specific T-cells are generated with peptides that recognize both class I and class II MHC molecules.

In some embodiments, the NY-ESO-1 peptides used to prime and expand a T-cell subpopulation includes specifically selected HLA-restricted peptides generated by determining the HLA profile of the donor source, and including peptides derived from NY-ESO-1 that best match the donor's HLA. In some embodiments, the NY-ESO-1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting NY-ESO-1 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 61-67, the HLA-B peptides are selected from the peptides of Tables 68-74, and the HLA-DR peptides are selected from the peptides of Tables 75-80. For example, if the donor cell source has an HLA profile that is HLA-A*01/*02:01; HLA-B*15:01/*18; and HLA-DRB1*0101/*0301, then the NY-ESO-1 peptides used to prime and expand the NY-ESO-1 specific T-cell subpopulation are restricted to the specific HLA profile, and may include the peptides identified in Table 61 (Seq. ID. Nos. 602-611) for HLA-A*01; Table 62 (Seq. ID. Nos. 612-621) for HLA-A*02:01; Table 70 (Seq. ID. Nos. 692-701) for HLA-B*15:01; Table 71 (Seq. ID. Nos. 702-711) for HLA-B*18; Table 75 (Seq. ID. Nos. 742-751) for HLA-DRB1*0101; and Table 76 (Seq. ID. Nos. 752-761) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the NY-ESO-1 targeted T-cell subpopulation is primed and expanded with one or more NY-ESO-1-derived peptides selected from Table 61 (Seq. ID. Nos. 602-611). In some embodiments, the donor cell source is HLA-A*01, and the NY-ESO-1 targeted T-cell subpopulation is primed and expanded with NY-ESO-1-derived peptides selected from Table 61 (Seq. ID. Nos. 602-611). In some embodiments, the donor cell source is HLA-A*01, and the NY-ESO-1 targeted T-cell subpopulation is primed and expanded with NY-ESO-1-derived peptides comprising the peptides of Table 61 (Seq. ID. Nos. 602-611). In some embodiments, the donor cell source is HLA-A*01, and the NY-ESO-1 targeted T-cell subpopulation is primed and expanded with NY-ESO-1-derived peptides comprising the peptides of Table 61 (Seq. ID. Nos. 602-611) and at least one additional set of peptides based on the donor cell source HLA-A profile, wherein the at least one additional set of peptides are selected from the peptides of Tables 62-67. In some embodiments, the NY-ESO-1-derived peptides also include one or more sets of HLA-B and HLA-DR restricted peptides selected from Tables 68-80 (Seq. ID Nos. 672-801).

TABLE 61
NYESO1 HLA-A*01 Epitope Peptides

| SEQ ID NO. | Sequence |
|---|---|
| 602 | RGPESRLLEFY |
| 603 | AADHRQLQLSI |
| 604 | EAELARRSLAQ |
| 605 | GPESRLLEFY |
| 606 | AQDAPPLPVP |
| 607 | AADHRQLQLS |
| 608 | EAELARRSLA |
| 609 | PESRLLEFY |
| 610 | AQDAPPLPV |
| 611 | AADHRQLQL |

In some embodiments, the donor cell source is HLA-A*02:01, and the NY-ESO-1 targeted T-cell subpopulation is primed and expanded with one or more NY-ESO-1-derived peptides selected from Table 62 (Seq. ID. Nos. 612-621). In some embodiments, the donor cell source is HLA-A*02:01, and the NY-ESO-1 targeted T-cell subpopulation is primed and expanded with NY-ESO-1-derived peptides selected from Table 62 (Seq. ID. Nos. 612-621). In some embodiments, the donor cell source is HLA-A*02:01, and the NY-ESO-1 targeted T-cell subpopulation is primed and expanded with NY-ESO-1-derived peptides comprising the peptides of Table 62 (Seq. ID. Nos. 612-621). In some embodiments, the donor cell source is HLA-A*02:01, and the NY-ESO-1 targeted T-cell subpopulation is primed and expanded with NY-ESO-1-derived peptides comprising the peptides of Table 62 (Seq. ID. Nos. 612-621) and at least one additional set of peptides based on the donor cell source HLA-A profile, wherein the at least one additional set of peptides are selected from the peptides of Tables 61, and 63-67. In some embodiments, the NY-ESO-1-derived peptides also include one or more sets of HLA-B and HLA-DR restricted peptides selected from Tables 68-80 (Seq. ID Nos. 672-801).

TABLE 62
NYESO1 HLA-A*02:01 Epitope Peptides

| SEQ ID NO. | Sequence |
|---|---|
| 612 | LLMWITQCFL |
| 613 | DAPPLPVPGV |
| 614 | RLLEFYLAMP |
| 615 | FTVSGNILTI |
| 616 | QLQLSISSCL |
| 617 | SLAQDAPPL |
| 618 | SISSCLQQL |
| 619 | RLLEFYLAM |
| 620 | TVSGNILTI |
| 621 | LMWITQCFL |

In some embodiments, the donor cell source is HLA-A*03, and the NY-ESO-1 targeted T-cell subpopulation is primed and expanded with one or more NY-ESO-1-derived peptides selected from Table 63 (Seq. ID. Nos. 622-631). In some embodiments, the donor cell source is HLA-A*03, and the NY-ESO-1 targeted T-cell subpopulation is primed and expanded with NY-ESO-1-derived peptides selected from Table 63 (Seq. ID. Nos. 622-631). In some embodiments, the donor cell source is HLA-A*03, and the NY-ESO-1 targeted T-cell subpopulation is primed and expanded with NY-ESO-1-derived peptides comprising the peptides of Table 63 (Seq. ID. Nos. 622-631). In some embodiments, the donor cell source is HLA-A*03, and the NY-ESO-1 targeted T-cell subpopulation is primed and expanded with NY-ESO-1-derived peptides comprising the peptides of Table 63 (Seq. ID. Nos. 622-631) and at least one additional set of peptides based on the donor cell source HLA-A profile, wherein the at least one additional set of peptides are selected from the peptides of Tables 61-62 and 64-67. In some embodiments, the NY-ESO-1-derived peptides also include one or more sets of HLA-B and HLA-DR restricted peptides selected from Tables 68-80 (Seq. ID Nos. 672-801).

TABLE 63
NYESO1 HLA-A*03 Epitope Peptides

| SEQ ID NO. | Sequence |
|---|---|
| 622 | PLPVPGVLLK |
| 623 | RLLEFYLAMP |
| 624 | ELARRSLAQD |
| 625 | TIRLTAADHR |
| 626 | RLTAADHRQL |
| 627 | QLSISSCLQQ |
| 628 | FLAQPPSGQR |
| 629 | TIRLTAADH |
| 630 | RLLEFYLAM |
| 631 | ELARRSLAQ |

In some embodiments, the donor cell source is HLA-A*11:01, and the NY-ESO-1 targeted T-cell subpopulation is primed and expanded with one or more NY-ESO-1-derived peptides selected from Table 64 (Seq. ID. Nos. 632-641). In some embodiments, the donor cell source is HLA-A*11:01, and the NY-ESO-1 targeted T-cell subpopulation is primed and expanded with NY-ESO-1-derived peptides selected from Table 64 (Seq. ID. Nos. 632-641). In some embodiments, the donor cell source is HLA-A*11:01, and the NY-ESO-1 targeted T-cell subpopulation is primed and expanded with NY-ESO-1-derived peptides comprising the peptides of Table 64 (Seq. ID. Nos. 632-641). In some embodiments, the donor cell source is HLA-A*11:01, and the NY-ESO-1 targeted T-cell subpopulation is primed and expanded with NY-ESO-1-derived peptides comprising the peptides of Table 64 (Seq. ID. Nos. 632-641), and at least one additional set of peptides based on the donor cell source HLA-A profile, wherein the at least one additional set of peptides are selected from the peptides of Tables 61-63 and 65-67. In some embodiments, the NY-ESO-1-derived peptides also include one or more sets of HLA-B and HLA-DR restricted peptides selected from Tables 68-80 (Seq. ID Nos. 672-801).

TABLE 64
NYESO1 HLA-A*11:01 Epitope Peptides

| SEQ ID NO. | Sequence |
|---|---|
| 632 | ATPMEAELAR |
| 633 | PLPVPGVLLK |
| 634 | ASGPGGGAPR |
| 635 | TVSGNILTIR |
| 636 | GVLLKEFTVS |
| 637 | ASGLNGCCR |
| 638 | LPVPGVLLK |
| 639 | VSGNILTIR |
| 640 | FTVSGNILT |
| 641 | SSCLQQLSL |

In some embodiments, the donor cell source is HLA-A*24:02, and the NY-ESO-1 targeted T-cell subpopulation is primed and expanded with one or more NY-ESO-1-derived peptides selected from Table 65 (Seq. ID. Nos. 642-651). In some embodiments, the donor cell source is HLA-A*24:02, and the NY-ESO-1 targeted T-cell subpopulation is primed and expanded with NY-ESO-1-derived peptides selected from Table 65 (Seq. ID. Nos. 642-651). In some embodiments, the donor cell source is HLA-A*24:02, and the NY-ESO-1 targeted T-cell subpopulation is primed and expanded with NY-ESO-1-derived peptides comprising the peptides of Table 65 (Seq. ID. Nos. 642-651). In some embodiments, the donor cell source is HLA-A*24:02, and the NY-ESO-1 targeted T-cell subpopulation is primed and expanded with NY-ESO-1-derived peptides comprising the peptides of Table 65 (Seq. ID. Nos. 642-651), and at least one additional set of peptides based on the donor cell source HLA-A profile, wherein the at least one additional set of peptides are selected from the peptides of Tables 61-64 and 66-67. In some embodiments, the NY-ESO-1-derived peptides also include one or more sets of HLA-B and HLA-DR restricted peptides selected from Tables 68-80 (Seq. ID Nos. 672-801).

TABLE 65
NYESO1 HLA-A*24:02 Epitope Peptides

| SEQ ID NO. | Sequence |
|---|---|
| 642 | PFATPMEAEL |
| 643 | PPLPVPGVLL |
| 644 | RGPESRLLEF |
| 645 | FYLAMPFATP |
| 646 | APPLPVPGVL |
| 647 | EFTVSGNIL |
| 648 | PPLPVPGVL |
| 649 | FYLAMPFAT |
| 650 | PLPVPGVLL |
| 651 | SCLQQLSLL |

In some embodiments, the donor cell source is HLA-A*26, and the NY-ESO-1 targeted T-cell subpopulation is primed and expanded with one or more NY-ESO-1-derived peptides selected from Table 66 (Seq. ID. Nos. 652-661). In some embodiments, the donor cell source is HLA-A*26, and the NY-ESO-1 targeted T-cell subpopulation is primed and expanded with NY-ESO-1-derived peptides selected from Table 66 (Seq. ID. Nos. 652-661). In some embodiments, the donor cell source is HLA-A*26, and the NY-ESO-1 targeted T-cell subpopulation is primed and expanded with NY-ESO-1-derived peptides comprising the peptides of Table 66 (Seq. ID. Nos. 652-661). In some embodiments, the donor cell source is HLA-A*26, and the NY-ESO-1 targeted T-cell subpopulation is primed and expanded with NY-ESO-1-derived peptides comprising the peptides of Table 66 (Seq. ID. Nos. 652-661) and at least one additional set of peptides based on the donor cell source HLA-A profile, wherein the at least one additional set of peptides are selected from the peptides of Tables 61-65 and 67. In some embodiments, the NY-ESO-1-derived peptides also include one or more sets of HLA-B and HLA-DR restricted peptides selected from Tables 68-80 (Seq. ID Nos. 672-801).

TABLE 66
NYESO1 HLA-A*26 Epitope Peptides

| SEQ ID NO. | Sequence |
|---|---|
| 652 | PVPGVLLKEF |
| 653 | FTVSGNILTI |
| 654 | LSISSCLQQL |
| 655 | WITQCFLPVF |
| 656 | EFTVSGNIL |
| 657 | ITQCFLPVF |
| 658 | ESRLLEFYL |
| 659 | EAELARRSL |
| 660 | SISSCLQQL |
| 661 | TVSGNILTI |

In some embodiments, the donor cell source is HLA-A*68:01, and the NY-ESO-1 targeted T-cell subpopulation is primed and expanded with one or more NY-ESO-1-derived peptides selected from Table 67 (Seq. ID. Nos. 662-671). In some embodiments, the donor cell source is HLA-A*68:01, and the NY-ESO-1 targeted T-cell subpopulation is primed and expanded with NY-ESO-1-derived peptides selected from Table 67 (Seq. ID. Nos. 662-671). In some embodiments, the donor cell source is HLA-A*68:01, and the NY-ESO-1 targeted T-cell subpopulation is primed and expanded with NY-ESO-1-derived peptides comprising the peptides of Table 67 (Seq. ID. Nos. 662-671). In some embodiments, the donor cell source is HLA-A*68:01, and the NY-ESO-1 targeted T-cell subpopulation is primed and expanded with NY-ESO-1-derived peptides comprising the peptides of Table 67 (Seq. ID. Nos. 662-671), and at least one additional set of peptides based on the donor cell source HLA-A profile, wherein the at least one additional set of peptides are selected from the peptides of Tables 61-66. In some embodiments, the NY-ESO-1-derived peptides also include one or more sets of HLA-B and HLA-DR restricted peptides selected from Tables 68-80 (Seq. ID Nos. 672-801).

TABLE 67
NYESO1 HLA-A*68:01 Epitope Peptides

| SEQ ID NO. | Sequence |
|---|---|
| 662 | ATPMEAELARR |
| 663 | FTVSGNILTIR |
| 664 | EAGATGGRGPR |
| 665 | LTIRLTAADHR |
| 666 | RASGPGGGAPR |
| 667 | TVSGNILTIR |
| 668 | ASGPGGGAPR |
| 669 | ATPMEAELAR |
| 670 | VSGNILTIR |
| 671 | PMEAELARR |

In some embodiments, the donor cell source is HLA-B*07:02, and the NY-ESO-1 targeted T-cell subpopulation is primed and expanded with one or more NY-ESO-1-derived peptides selected from Table 68 (Seq. ID. Nos. 672-681). In some embodiments, the donor cell source is HLA-B*07:02, and the NY-ESO-1 targeted T-cell subpopulation is primed and expanded with NY-ESO-1-derived peptides selected from Table 68 (Seq. ID. Nos. 672-681). In some embodiments, the donor cell source is HLA-B*07:02, and the NY-ESO-1 targeted T-cell subpopulation is primed and expanded with NY-ESO-1-derived peptides comprising the peptides of Table 68 (Seq. ID. Nos. 672-681). In some embodiments, the donor cell source is HLA-B*07:02, and the NY-ESO-1 targeted T-cell subpopulation is primed and expanded with NY-ESO-1-derived peptides comprising the peptides of Table 68 (Seq. ID. Nos. 672-681), and at least one additional set of peptides based on the donor cell source HLA-B profile, wherein the at least one additional set of peptides are selected from the peptides of Tables 69-74. In some embodiments, the NY-ESO-1-derived peptides also include one or more sets of HLA-A and HLA-DR restricted peptides selected from Tables 61-67 and 75-80 (Seq. ID Nos. 602-671 and 742-801).

TABLE 68
NYESO1 HLA-B*07:02 Epitope Peptides

| SEQ ID NO. | Sequence |
|---|---|
| 672 | APRGPHGGAA |
| 673 | APPLPVPGVL |
| 674 | PPLPVPGVLL |
| 675 | GPHGGAASGL |
| 676 | GPRGAGAARA |
| 677 | APRGPHGGA |
| 678 | IPDGPGGNA |
| 679 | APPLPVPGV |
| 680 | PPLPVPGVL |
| 681 | GPGGPGIPD |

In some embodiments, the donor cell source is HLA-B*08, and the NY-ESO-1 targeted T-cell subpopulation is primed and expanded with one or more NY-ESO-1-derived peptides selected from Table 69 (Seq. ID. Nos. 682-691). In some embodiments, the donor cell source is HLA-B*08, and the NY-ESO-1 targeted T-cell subpopulation is primed and expanded with NY-ESO-1-derived peptides selected from Table 69 (Seq. ID. Nos. 682-691). In some embodiments, the donor cell source is HLA-B*08, and the NY-ESO-1 targeted T-cell subpopulation is primed and expanded with NY-ESO-1-derived peptides comprising the peptides of Table 69 (Seq. ID. Nos. 682-691). In some embodiments, the donor cell source is HLA-B*08, and the NY-ESO-1 targeted T-cell subpopulation is primed and expanded with NY-ESO-1-derived peptides comprising the peptides of Table 69 (Seq. ID. Nos. 682-691) and at least one additional set of peptides based on the donor cell source HLA-B profile, wherein the at least one additional set of peptides are selected from the peptides of Tables 68 and 70-74. In some embodiments, the NY-ESO-1-derived peptides also include one or more sets of HLA-A and HLA-DR restricted peptides selected from Tables 61-67 and 75-80 (Seq. ID Nos. 602-671 and 742-801).

TABLE 69
NYESO1 HLA-B*08 Epitope Peptides

| SEQ ID NO. | Sequence |
|---|---|
| 682 | GPESRLLEF |
| 683 | AADHRQLQL |
| 684 | GARGPESRL |
| 685 | ESRLLEFYL |
| 686 | LLKEFTVSG |
| 687 | SLAQDAPPL |
| 688 | PLPVPGVLL |
| 689 | AELARRSL |
| 690 | LLKEFTVS |
| 691 | PLPVPGVL |

In some embodiments, the donor cell source is HLA-B*15:01, and the NY-ESO-1 targeted T-cell subpopulation is primed and expanded with one or more NY-ESO-1-derived peptides selected from Table 70 (Seq. ID. Nos. 692-701). In some embodiments, the donor cell source is HLA-B*15:01, and the NY-ESO-1 targeted T-cell subpopulation is primed and expanded with NY-ESO-1-derived peptides selected from Table 70 (Seq. ID. Nos. 692-701). In some embodiments, the donor cell source is HLA-B*15:01, and the NY-ESO-1 targeted T-cell subpopulation is primed and expanded with NY-ESO-1-derived peptides comprising the peptides of Table 70 (Seq. ID. Nos. 692-701). In some embodiments, the donor cell source is HLA-B*15:01, and the NY-ESO-1 targeted T-cell subpopulation is primed and expanded with NY-ESO-1-derived peptides comprising the peptides of Table 70 (Seq. ID. Nos. 692-701) and at least one additional set of peptides based on the donor cell source HLA-B profile, wherein the at least one additional set of peptides are selected from the peptides of Tables 68-69 and 71-74. In some embodiments, the NY-ESO-1-derived peptides also include one or more sets of HLA-A and HLA-DR restricted peptides selected from Tables 61-67 and 75-80 (Seq. ID Nos. 602-671 and 742-801).

TABLE 70
NYESO1 HLA-B*15:01 (B62) Epitope Peptides

| SEQ ID NO. | Sequence |
|---|---|
| 692 | SLLMWITQCF |
| 693 | PVPGVLLKEF |
| 694 | LLEFYLAMPF |
| 695 | RLLEFYLAMP |
| 696 | VLLKEFTVSG |
| 697 | MQAEGRGTGG |
| 698 | ILTIRLTAAD |
| 699 | RQLQLSISSC |
| 700 | LLMWITQCF |
| 701 | LLKEFTVSG |

In some embodiments, the donor cell source is HLA-B*18, and the NY-ESO-1 targeted T-cell subpopulation is primed and expanded with one or more NY-ESO-1-derived peptides selected from Table 71 (Seq. ID. Nos. 702-711). In some embodiments, the donor cell source is HLA-B*18, and the NY-ESO-1 targeted T-cell subpopulation is primed and expanded with NY-ESO-1-derived peptides selected from Table 71 (Seq. ID. Nos. 702-711). In some embodiments, the donor cell source is HLA-B*18, and the NY-ESO-1 targeted T-cell subpopulation is primed and expanded with NY-ESO-1-derived peptides comprising the peptides of Table 71 (Seq. ID. Nos. 702-711). In some embodiments, the donor cell source is HLA-B*18, and the NY-ESO-1 targeted T-cell subpopulation is primed and expanded with NY-ESO-1-derived peptides comprising the peptides of Table 71 (Seq. ID. Nos. 702-711) and at least one additional set of peptides based on the donor cell source HLA-B profile, wherein the at least one additional set of peptides are selected from the peptides of Tables 68-70 and 72-74. In some embodiments, the NY-ESO-1-derived peptides also include one or more sets of HLA-A and HLA-DR restricted peptides selected from Tables 61-67 and 75-80 (Seq. ID Nos. 602-671 and 742-801).

TABLE 71
NYESO1 HLA-B*18 Epitope Peptides

| SEQ ID NO. | Sequence |
|---|---|
| 702 | PESRLLEFY |
| 703 | LEFYLAMPF |
| 704 | MEAELARRS |
| 705 | ESRLLEFYL |
| 706 | VPGVLLKEF |
| 707 | ITQCFLPVF |
| 708 | PESRLLEF |
| 709 | AELARRSL |
| 710 | PGVLLKEF |
| 711 | MEAELARR |

In some embodiments, the donor cell source is HLA-B*27:05, and the NY-ESO-1 targeted T-cell subpopulation is primed and expanded with one or more NY-ESO-1-derived peptides selected from Table 72 (Seq. ID. Nos. 712-721). In some embodiments, the donor cell source is HLA-B*27:05, and the NY-ESO-1 targeted T-cell subpopulation is primed and expanded with NY-ESO-1-derived peptides selected from Table 72 (Seq. ID. Nos. 712-721). In some embodiments, the donor cell source is HLA-B*27:05, and the NY-ESO-1 targeted T-cell subpopulation is primed and expanded with NY-ESO-1-derived peptides comprising the peptides of Table 72 (Seq. ID. Nos. 712-721). In some embodiments, the donor cell source is HLA-B*27:05, and the NY-ESO-1 targeted T-cell subpopulation is primed and expanded with NY-ESO-1-derived peptides comprising the peptides of Table 72 (Seq. ID. Nos. 712-721) and at least one additional set of peptides based on the donor cell source HLA-B profile, wherein the at least one additional set of peptides are selected from the peptides of Tables 68-71 and 73-74. In some embodiments, the NY-ESO-1-derived peptides also include one or more sets of HLA-A and HLA-DR restricted peptides selected from Tables 61-67 and 75-80 (Seq. ID Nos. 602-671 and 742-801).

TABLE 72
NYESO1 HLA-B*27:05 Epitope Peptides

| SEQ ID NO. | Sequence |
|---|---|
| 712 | SRLLEFYLAM |
| 713 | RGPESRLLEF |
| 714 | RSLAQDAPPL |
| 715 | GPHGGAASGL |
| 716 | RRSLAQDAPP |
| 717 | ARGPESRLL |
| 718 | IRLTAADHR |
| 719 | GARGPESRL |
| 720 | GRGTGGSTG |
| 721 | GATGGRGPR |

In some embodiments, the donor cell source is HLA-B*35:01, and the NY-ESO-1 targeted T-cell subpopulation is primed and expanded with one or more NY-ESO-1-derived peptides selected from Table 73 (Seq. ID. Nos. 722-731). In some embodiments, the donor cell source is HLA-B*35:01, and the NY-ESO-1 targeted T-cell subpopulation is primed and expanded with NY-ESO-1-derived peptides selected from Table 73 (Seq. ID. Nos. 722-731). In some embodiments, the donor cell source is HLA-B*35:01, and the NY-ESO-1 targeted T-cell subpopulation is primed and expanded with NY-ESO-1-derived peptides comprising the peptides of Table 73 (Seq. ID. Nos. 722-731). In some embodiments, the donor cell source is HLA-B*35:01, and the NY-ESO-1 targeted T-cell subpopulation is primed and expanded with NY-ESO-1-derived peptides comprising the peptides of Table 73 (Seq. ID. Nos. 722-731) and at least one additional set of peptides based on the donor cell source HLA-B profile, wherein the at least one additional set of peptides are selected from the peptides of Tables 68-72 and 74. In some embodiments, the NY-ESO-1-derived peptides also include one or more sets of HLA-A and HLA-DR restricted peptides selected from Tables 61-67 and 75-80 (Seq. ID Nos. 602-671 and 742-801).

TABLE 73
NYESO1 HLA-B*35:01 Epitope Peptides

| SEQ ID NO. | Sequence |
|---|---|
| 722 | PPLPVPGVLL |
| 723 | GPESRLLEFY |
| 724 | GPHGGAASGL |
| 725 | APPLPVPGVL |
| 726 | MPFATPMEAE |
| 727 | PPLPVPGVL |
| 728 | GPESRLLEF |
| 729 | VPGVLLKEF |
| 730 | LQLSISSCL |
| 731 | LPVFLAQPP |

In some embodiments, the donor cell source is HLA-B*58:02, and the NY-ESO-1 targeted T-cell subpopulation is primed and expanded with one or more NY-ESO-1-derived peptides selected from Table 74 (Seq. ID. Nos. 732-741). In some embodiments, the donor cell source is HLA-B*58:02, and the NY-ESO-1 targeted T-cell subpopulation is primed and expanded with NY-ESO-1-derived peptides selected from Table 74 (Seq. ID. Nos. 732-741). In some embodiments, the donor cell source is HLA-B*58:02, and the NY-ESO-1 targeted T-cell subpopulation is primed and expanded with NY-ESO-1-derived peptides comprising the peptides of Table 74 (Seq. ID. Nos. 732-741). In some embodiments, the donor cell source is HLA-B*58:02, and the NY-ESO-1 targeted T-cell subpopulation is primed and expanded with NY-ESO-1-derived peptides comprising the peptides of Table 74 (Seq. ID. Nos. 732-741) and at least one additional set of peptides based on the donor cell source HLA-B profile, wherein the at least one additional set of peptides are selected from the peptides of Tables 68-73. In some embodiments, the NY-ESO-1-derived peptides also include one or more sets of HLA-A and HLA-DR restricted peptides selected from Tables 61-67 and 75-80 (Seq. ID Nos. 602-671 and 742-801).

TABLE 74
NYESO1 HLA-B*58:02 Epitope Peptides

| SEQ ID NO. | Sequence |
|---|---|
| 732 | RSLAQDAPPL |
| 733 | GARGPESRLL |
| 734 | FTVSGNILTI |
| 735 | LSISSCLQQL |
| 736 | SSCLQQLSLL |
| 737 | VSGNILTIRL |
| 738 | ISSCLQQLSL |
| 739 | EAELARRSL |
| 740 | LTAADHRQL |
| 741 | ESRLLEFYL |

In some embodiments, the donor cell source is HLA-DRB1*0101, and the NY-ESO-1 targeted T-cell subpopulation is primed and expanded with one or more NY-ESO-1-derived peptides selected from Table 75 (Seq. ID. Nos. 742-751). In some embodiments, the donor cell source is HLA-DRB1*0101, and the NY-ESO-1 targeted T-cell subpopulation is primed and expanded with NY-ESO-1-derived peptides selected from Table 75 (Seq. ID. Nos. 742-751). In some embodiments, the donor cell source is HLA-DRB1*0101, and the NY-ESO-1 targeted T-cell subpopulation is primed and expanded with NY-ESO-1-derived peptides comprising the peptides of Table 75 (Seq. ID. Nos. 742-751). In some embodiments, the donor cell source is HLA-DRB1*0101, and the NY-ESO-1 targeted T-cell subpopulation is primed and expanded with NY-ESO-1-derived peptides comprising the peptides of Table 75 (Seq. ID. Nos. 742-751) and at least one additional set of peptides based on the donor cell source HLA-DR profile, wherein the at least one additional set of peptides are selected from the peptides of Tables 76-80. In some embodiments, the NY-ESO-1-derived peptides also include one or more sets of HLA-A and HLA-B restricted peptides selected from Tables 61-74 (Seq. ID Nos. 602-741).

TABLE 75
NYESO1 HLA-DRB1*0101 Epitope Peptides

| SEQ ID NO. | Sequence |
|---|---|
| 742 | EFYLAMPFATPMEAE |
| 743 | SRLLEFYLAMPFATP |
| 744 | ATPMEAELARRSLAQ |
| 745 | GPGIPDGPGGNAGGP |
| 746 | LEFYLAMPFATPMEA |
| 747 | MPFATPMEAELARRS |
| 748 | LLMWITQCFLPVFLA |
| 749 | TQCFLPVFLAQPPSG |
| 750 | QCFLPVFLAQPPSGQ |
| 751 | YLAMPFATPMEAELA |

In some embodiments, the donor cell source is HLA-DRB1*0301, and the NY-ESO-1 targeted T-cell subpopulation is primed and expanded with one or more NY-ESO-1-derived peptides selected from Table 76 (Seq. ID. Nos. 752-761). In some embodiments, the donor cell source is HLA-DRB1*0301, and the NY-ESO-1 targeted T-cell subpopulation is primed and expanded with NY-ESO-1-derived peptides selected from Table 76 (Seq. ID. Nos. 752-761). In some embodiments, the donor cell source is HLA-DRB1*0301, and the NY-ESO-1 targeted T-cell subpopulation is primed and expanded with NY-ESO-1-derived peptides comprising the peptides of Table 76 (Seq. ID. Nos. 752-761). In some embodiments, the donor cell source is HLA-DRB1*0301, and the NY-ESO-1 targeted T-cell subpopulation is primed and expanded with NY-ESO-1-derived peptides comprising the peptides of Table 76 (Seq. ID. Nos. 752-761) and at least one additional set of peptides based on the donor cell source HLA-DR profile, wherein the at least one additional set of peptides are selected from the peptides of Tables 75 and 77-80. In some embodiments, the NY-ESO-1-derived peptides also include one or more sets of HLA-A and HLA-B restricted peptides selected from Tables 61-74 (Seq. ID Nos. 602-741).

TABLE 76
NYESO1 HLA-DRB1*0301 (DR17) Epitope Peptides

| SEQ ID NO. | Sequence |
|---|---|
| 752 | LSLLMWITQCFLPVF |
| 753 | AMPFATPMEAELARR |
| 754 | QLSLLMWITQCFLPV |
| 755 | RRSLAQDAPPLPVPG |
| 756 | QLSISSCLQQLSLLM |
| 757 | SRLLEFYLAMPFATP |
| 758 | PLPVPGVLLKEFTVS |
| 759 | TIRLTAADHRQLQLS |
| 760 | HRQLQLSISSCLQQL |
| 761 | LMWITQCFLPVFLAQ |

In some embodiments, the donor cell source is HLA-DRB1*0401, and the NY-ESO-1 targeted T-cell subpopulation is primed and expanded with one or more NY-ESO-1-derived peptides selected from Table 77 (Seq. ID. Nos. 762-771). In some embodiments, the donor cell source is HLA-DRB1*0401, and the NY-ESO-1 targeted T-cell subpopulation is primed and expanded with NY-ESO-1-derived peptides selected from Table 77 (Seq. ID. Nos. 762-771). In some embodiments, the donor cell source is HLA-DRB1*0401, and the NY-ESO-1 targeted T-cell subpopulation is primed and expanded with NY-ESO-1-derived peptides comprising the peptides of Table 77 (Seq. ID. Nos. 762-771). In some embodiments, the donor cell source is HLA-DRB1*0401, and the NY-ESO-1 targeted T-cell subpopulation is primed and expanded with NY-ESO-1-derived peptides comprising the peptides of Table 77 (Seq. ID. Nos. 762-771) and at least one additional set of peptides based on the donor cell source HLA-DR profile, wherein the at least one additional set of peptides are selected from the peptides of Tables 75-76 and 78-80. In some embodiments, the NY-ESO-1-derived peptides also include one or more sets of HLA-A and HLA-B restricted peptides selected from Tables 61-74 (Seq. ID Nos. 602-741).

TABLE 77
NYESO1 HLA-DRB1*0401 (DR4Dw4) Epitope Peptides

| SEQ ID NO. | Sequence |
|---|---|
| 762 | TIRLTAADHRQLQLS |
| 763 | LSLLMWITQCFLPVF |
| 764 | LLEFYLAMPFATPME |
| 765 | LKEFTVSGNILTIRL |
| 766 | ASGLNGCCRCGARGP |
| 767 | YLAMPFATPMEAELA |
| 768 | ATPMEAELARRSLAQ |
| 769 | PGVLLKEFTVSGNIL |
| 770 | GVLLKEFTVSGNILT |
| 771 | SGNILTIRLTAADHR |

In some embodiments, the donor cell source is HLA-DRB1*0701, and the NY-ESO-1 targeted T-cell subpopulation is primed and expanded with one or more NY-ESO-1-derived peptides selected from Table 78 (Seq. ID. Nos. 772-781). In some embodiments, the donor cell source is HLA-DRB1*0701, and the NY-ESO-1 targeted T-cell subpopulation is primed and expanded with NY-ESO-1-derived peptides selected from Table 78 (Seq. ID. Nos. 772-781). In some embodiments, the donor cell source is HLA-DRB1*0701, and the NY-ESO-1 targeted T-cell subpopulation is primed and expanded with NY-ESO-1-derived peptides comprising the peptides of Table 78 (Seq. ID. Nos. 772-781). In some embodiments, the donor cell source is HLA-DRB1*0701, and the NY-ESO-1 targeted T-cell subpopulation is primed and expanded with NY-ESO-1-derived peptides comprising the peptides of Table 78 (Seq. ID. Nos. 772-781) and at least one additional set of peptides based on the donor cell source HLA-DR profile, wherein the at least one additional set of peptides are selected from the peptides of Tables 75-77 and 79-80. In some embodiments, the NY-ESO-1-derived peptides also include one or more sets of HLA-A and HLA-B restricted peptides selected from Tables 61-74 (Seq. ID Nos. 602-741).

TABLE 78
NYESO1 HLA-DRB1*0701 Epitope Peptides

| SEQ ID NO. | Sequence |
|---|---|
| 772 | HRQLQLSISSCLQQL |
| 773 | AMPFATPMEAELARR |
| 774 | VLLKEFTVSGNILTI |
| 775 | LKEFTVSGNILTIRL |
| 776 | FTVSGNILTIRLTAA |
| 777 | TIRLTAADHRQLQLS |
| 778 | QLSLLMWITQCFLPV |
| 779 | LSLLMWITQCFLPVF |
| 780 | YLAMPFATPMEAELA |
| 781 | SGNILTIRLTAADHR |

In some embodiments, the donor cell source is HLA-DRB1*1101, and the NY-ESO-1 targeted T-cell subpopulation is primed and expanded with one or more NY-ESO-1-derived peptides selected from Table 79 (Seq. ID. Nos. 782-791). In some embodiments, the donor cell source is HLA-DRB1*1101, and the NY-ESO-1 targeted T-cell subpopulation is primed and expanded with NY-ESO-1-derived peptides selected from Table 79 (Seq. ID. Nos. 782-791). In some embodiments, the donor cell source is HLA-DRB1*1101, and the NY-ESO-1 targeted T-cell subpopulation is primed and expanded with NY-ESO-1-derived peptides comprising the peptides of Table 79 (Seq. ID. Nos. 782-791). In some embodiments, the donor cell source is HLA-DRB1*1101, and the NY-ESO-1 targeted T-cell subpopulation is primed and expanded with NY-ESO-1-derived peptides comprising the peptides of Table 79 (Seq. ID. Nos. 782-791) and at least one additional set of peptides based on the donor cell source HLA-DR profile, wherein the at least one additional set of peptides are selected from the peptides of Tables 75-78 and 80. In some embodiments, the NY-ESO-1-derived peptides also include one or more sets of HLA-A and HLA-B restricted peptides selected from Tables 61-74 (Seq. ID Nos. 602-741).

TABLE 79
NYESO1 HLA-DRB1*1101 Epitope Peptides

| SEQ ID NO. | Sequence |
|---|---|
| 782 | LEFYLAMPFATPMEA |
| 783 | TQCFLPVFLAQPPSG |
| 784 | ASGLNGCCRCGARGP |
| 785 | SGNILTIRLTAADHR |
| 786 | TIRLTAADHRQLQLS |
| 787 | MPFATPMEAELARRS |
| 788 | ATPMEAELARRSLAQ |
| 789 | TPMEAELARRSLAQD |
| 790 | PMEAELARRSLAQDA |
| 791 | LPVPGVLLKEFTVSG |

In some embodiments, the donor cell source is HLA-DRB1*1501, and the NY-ESO-1 targeted T-cell subpopulation is primed and expanded with one or more NY-ESO-1-derived peptides selected from Table 80 (Seq. ID. Nos. 792-801). In some embodiments, the donor cell source is HLA-DRB1*1501, and the NY-ESO-1 targeted T-cell subpopulation is primed and expanded with NY-ESO-1-derived peptides selected from T Table 80 (Seq. ID. Nos. 792-801). In some embodiments, the donor cell source is HLA-DRB1*1501, and the NY-ESO-1 targeted T-cell subpopulation is primed and expanded with NY-ESO-1-derived peptides comprising the peptides of Table 80 (Seq. ID. Nos. 792-801). In some embodiments, the donor cell source is HLA-DRB1*1501, and the NY-ESO-1 targeted T-cell subpopulation is primed and expanded with NY-ESO-1-derived peptides comprising the peptides of Table 80 (Seq. ID. Nos. 792-801) and at least one additional set of peptides based on the donor cell source HLA-DR profile, wherein the at least one additional set of peptides are selected from the peptides of Tables 75-79. In some embodiments, the NY-ESO-1-derived peptides also include one or more sets of HLA-A and HLA-B restricted peptides selected from Tables 61-74 (Seq. ID Nos. 602-741).

TABLE 80
NYESO1 HLA-DRB1*1501 (DR2b) Epitope Peptides

| SEQ ID NO. | Sequence |
|---|---|
| 792 | SRLLEFYLAMPFATP |
| 793 | QCFLPVFLAQPPSGQ |
| 794 | ESRLLEFYLAMPFAT |
| 795 | YLAMPFATPMEAELA |
| 796 | PGVLLKEFTVSGNIL |
| 797 | GVLLKEFTVSGNILT |
| 798 | QLSLLMWITQCFLPV |
| 799 | MWITQCFLPVFLAQP |
| 800 | LLEFYLAMPFATPME |
| 801 | LKEFTVSGNILTIRL |

MAGE A3 Antigenic Peptides

In some embodiments, the MUSTANG composition includes MAGE-A3 (Melanoma-associated antigen 3) specific T-cells. MAGE-A3 specific T-cells can be generated as described below using one or more antigenic peptides to MAGE-A3. In some embodiments, the MAGE-A3 specific T-cells are generated using one or more antigenic peptides to MAGE-A3, or a modified or heteroclitic peptide derived from a MAGE-A3 peptide. In some embodiments, MAGE-A3 specific T-cells are generated using a MAGE-A3 antigen library comprising a pool of peptides (for example 15mers) containing amino acid overlap (for example 11 amino acids of overlap) between each sequence formed by scanning the protein amino acid sequence SEQ. ID. No. 802 (UniProt KB-P43357) for MAGE-A3:

MPLEQRSQHCKPEEGLEARGEALGLVGAQAPATEEQEAASSSSTLVEVT
LGEVPAAESPDPPQSPQGASSLPTTMNYPLWSQSYEDSSNQEEEGPSTF
PDLESEFQAALSRKVAELVHFLLLKYRAREPVTKAEMLGSVVGNWQYFF
PVILLIIVLAIIAREGDCAPEEKIWEELSVLEVFEGREDSILGDPKKLL
TQHFVQENYLEYRQVPGSDPACYEFLWGPRALVETSYVKVLHHMVKISG
GPHISYPPLHEWVLREGEE.

Overlapping antigenic libraries are commercially available, for example, from JPT, for example, from JPT (Product Code: PM-MAGEA3 (Pep Mix™ Human (MAGE-A3)). In some embodiments, the MAGE-A3 specific T-cells are generated using a commercially available overlapping antigenic library made up of MAGE-A3 peptides.

In some embodiments, the MAGE-A3 specific T-cells are generated using one or more antigenic peptides to MAGE-A3, or a modified or heteroclitic peptide derived from a MAGE-A3 peptide. In some embodiments, the MAGE-A3 specific T-cells are generated with peptides that recognize class I MHC molecules. In some embodiments, the MAGE-A3 specific T-cells are generated with peptides that recognize class II MEW molecules. In some embodiments, the MAGE-A3 specific T-cells are generated with peptides that recognize both class I and class II MEW molecules.

In some embodiments, the MAGE-A3 peptides used to prime and expand a T-cell subpopulation includes specifically selected HLA-restricted peptides generated by determining the HLA profile of the donor source, and including peptides derived from MAGE-A3 that best match the donor's HLA. In some embodiments, the MAGE-A3 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting MAGE-A3 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 81-87, the HLA-B peptides are selected from the peptides of Tables 88-94, and the HLA-DR peptides are selected from the peptides of Tables 95-100. For example, if the donor cell source has an HLA profile that is HLA-A*01/*02:01; HLA-B*15:01/*18; and HLA-DRB1*0101/*0301, then the MAGE-A3 peptides used to prime and expand the MAGE-A3 specific T-cell subpopulation are restricted to the specific HLA profile, and may include the peptides identified in Table 81 (Seq. ID. Nos. 803-812) for HLA-A*01; Table 82 (Seq. ID. Nos. 813-822) for HLA-A*02:01; Table 90 (Seq. ID. Nos. 893-902) for HLA-B*15:01; Table 91 (Seq. ID. Nos. 903-912) for HLA-B*18; Table 95 (Seq. ID. Nos. 943-952) for HLA-DRB1*0101; and Table 96 (Seq. ID. Nos. 953-962) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the MAGE-A3 targeted T-cell subpopulation is primed and expanded with one or more MAGE-A3-derived peptides selected from Table 81 (Seq. ID. Nos. 803-812). In some embodiments, the donor cell source is HLA-A*01, and the MAGE-A3 targeted T-cell subpopulation is primed and expanded with MAGE-A3-derived peptides selected from Table 81 (Seq. ID. Nos. 803-812). In some embodiments, the donor cell source is HLA-A*01, and the MAGE-A3 targeted T-cell subpopulation is primed and expanded with MAGE-A3-derived peptides comprising the peptides of Table 81 (Seq. ID. Nos. 803-812). In some embodiments, the donor cell source is HLA-A*01, and the MAGE-A3 targeted T-cell subpopulation is primed and expanded with MAGE-A3-derived peptides comprising the peptides of Table 81 (Seq. ID. Nos. 803-812) and at least one additional set of peptides based on the donor cell source HLA-A profile, wherein the at least one additional set of peptides are selected from the peptides of Tables 82-87. In some embodiments, the MAGE-A3-derived peptides also include one or more sets of HLA-B and HLA-DR restricted peptides selected from Tables 88-100 (Seq. ID Nos. 873-1002).

TABLE 81
MAGEA3 HLA-A*01 Epitope Peptides

| SEQ ID NO. | Sequence |
|---|---|
| 803 | LMEVDPIGHLY |
| 804 | AELVHFLLLKY |
| 805 | QHFVQENYLEY |
| 806 | ASSLPTTMNY |
| 807 | ELVHFLLLKY |
| 808 | LTQHFVQENY |
| 809 | EVDPIGHLY |
| 810 | SSLPTTMNY |
| 811 | LVHFLLLKY |
| 812 | GSVVGNWQY |

In some embodiments, the donor cell source is HLA-A*02:01, and the MAGE-A3 targeted T-cell subpopulation is primed and expanded with one or more MAGE-A3-derived peptides selected from Table 82 (Seq. ID. Nos. 813-822). In some embodiments, the donor cell source is HLA-A*02:01, and the MAGE-A3 targeted T-cell subpopulation is primed and expanded with MAGE-A3-derived peptides selected from Table 82 (Seq. ID. Nos. 813-822). In some embodiments, the donor cell source is HLA-A*02:01, and the MAGE-A3 targeted T-cell subpopulation is primed and expanded with MAGE-A3-derived peptides comprising the peptides of Table 82 (Seq. ID. Nos. 813-822). In some embodiments, the donor cell source is HLA-A*02:01, and the MAGE-A3 targeted T-cell subpopulation is primed and expanded with MAGE-A3-derived peptides comprising the peptides of Table 82 (Seq. ID. Nos. 813-822) and at least one additional set of peptides based on the donor cell source HLA-A profile, wherein the at least one additional set of peptides are selected from the peptides of Tables 81, and 83-87. In some embodiments, the MAGE-A3-derived peptides also include one or more sets of HLA-B and HLA-DR restricted peptides selected from Tables 88-100 (Seq. ID Nos. 873-1002).

TABLE 82
MAGEA3 HLA-A*02:01 Epitope Peptides

| SEQ ID NO. | Sequence |
|---|---|
| 813 | TLVEVTLGEV |
| 814 | ALVETSYVKV |
| 815 | GLLIIVLAII |
| 816 | AALSRKVAEL |
| 817 | LVFGIELMEV |
| 818 | ALSRKVAEL |
| 819 | LLIIVLAII |
| 820 | GLLIIVLAI |
| 821 | FLWGPRALV |
| 822 | KIWEELSVL |

In some embodiments, the donor cell source is HLA-A*03, and the MAGE-A3 targeted T-cell subpopulation is primed and expanded with one or more MAGE-A3-derived peptides selected from Table 83 (Seq. ID. Nos. 823-832). In some embodiments, the donor cell source is HLA-A*03, and the MAGE-A3 targeted T-cell subpopulation is primed and expanded with MAGE-A3-derived peptides selected from Table 83 (Seq. ID. Nos. 823-832). In some embodiments, the donor cell source is HLA-A*03, and the MAGE-A3 targeted T-cell subpopulation is primed and expanded with MAGE-A3-derived peptides comprising the peptides of Table 83 (Seq. ID. Nos. 823-832). In some embodiments, the donor cell source is HLA-A*03, and the MAGE-A3 targeted T-cell subpopulation is primed and expanded with MAGE-A3-derived peptides comprising the peptides of Table 83 (Seq. ID. Nos. 823-832) and at least one additional set of peptides based on the donor cell source HLA-A profile, wherein the at least one additional set of peptides are selected from the peptides of Tables 81-82 and 84-87. In some embodiments, the MAGE-A3-derived peptides also include one or more sets of HLA-B and HLA-DR restricted peptides selected from Tables 88-100 (Seq. ID Nos. 873-1002).

TABLE 83
MAGEA3 HLA-A*03 Epitope Peptides

| SEQ ID NO. | Sequence |
|---|---|
| 823 | KYRAREPVTK |
| 824 | YVKVLHHMVK |
| 825 | QVPGSDPACY |
| 826 | LLGDNQIMPK |
| 827 | KLLTQHFVQE |
| 828 | FLWGPRALVE |
| 829 | ALVETSYVK |
| 830 | ALGLVGAQA |
| 831 | ELVHFLLLK |
| 832 | YRAREPVTK |

In some embodiments, the donor cell source is HLA-A*11:01, and the MAGE-A3 targeted T-cell subpopulation is primed and expanded with one or more MAGE-A3-derived peptides selected from Table 84 (Seq. ID. Nos. 833-842). In some embodiments, the donor cell source is HLA-A*11:01, and the MAGE-A3 targeted T-cell subpopulation is primed and expanded with MAGE-A3-derived peptides selected from Table 84 (Seq. ID. Nos. 833-842). In some embodiments, the donor cell source is HLA-A*11:01, and the MAGE-A3 targeted T-cell subpopulation is primed and expanded with MAGE-A3-derived peptides comprising the peptides of Table 84 (Seq. ID. Nos. 833-842). In some embodiments, the donor cell source is HLA-A*11:01, and the MAGE-A3 targeted T-cell subpopulation is primed and expanded with MAGE-A3-derived peptides comprising the peptides of Table 84 (Seq. ID. Nos. 833-842), and at least one additional set of peptides based on the donor cell source HLA-A profile, wherein the at least one additional set of peptides are selected from the peptides of Tables 81-83 and 85-87. In some embodiments, the MAGE-A3-derived peptides also include one or more sets of HLA-B and HLA-DR restricted peptides selected from Tables 88-100 (Seq. ID Nos. 873-1002).

TABLE 84
MAGEA3 HLA-A*11:01 Epitope Peptides

| SEQ ID NO. | Sequence |
|---|---|
| 833 | ESEFQAALSR |
| 834 | YVKVLHHMVK |
| 835 | AELVHFLLLK |
| 836 | LIIVLAIIAR |
| 837 | ASSSSTLVEV |
| 838 | STLVEVTLGE |
| 839 | ELVHFLLLK |
| 840 | SVLEVFEGR |
| 841 | DSILGDPKK |
| 842 | ALVETSYVK |

In some embodiments, the donor cell source is HLA-A*24:02, and the MAGE-A3 targeted T-cell subpopulation is primed and expanded with one or more MAGE-A3-derived peptides selected from Table 85 (Seq. ID. Nos. 843-852). In some embodiments, the donor cell source is HLA-A*24:02, and the MAGE-A3 targeted T-cell subpopulation is primed and expanded with MAGE-A3-derived peptides selected from Table 85 (Seq. ID. Nos. 843-852). In some embodiments, the donor cell source is HLA-A*24:02, and the MAGE-A3 targeted T-cell subpopulation is primed and expanded with MAGE-A3-derived peptides comprising the peptides of Table 85 (Seq. ID. Nos. 843-852). In some embodiments, the donor cell source is HLA-A*24:02, and the MAGE-A3 targeted T-cell subpopulation is primed and expanded with MAGE-A3-derived peptides comprising the peptides of Table 85 (Seq. ID. Nos. 843-852), and at least one additional set of peptides based on the donor cell source HLA-A profile, wherein the at least one additional set of peptides are selected from the peptides of Tables 81-84 and 86-87. In some embodiments, the MAGE-A3-derived peptides also include one or more sets of HLA-B and HLA-DR restricted peptides selected from Tables 88-100 (Seq. ID Nos. 873-1002).

TABLE 85
MAGEA3 HLA-A*24:02 Epitope Peptides

| SEQ ID NO. | Sequence |
|---|---|
| 843 | SYPPLHEWVL |
| 844 | LYIFATCLGL |
| 845 | VFEGREDSIL |
| 846 | KVAELVHFLL |
| 847 | TFPDLESEF |
| 848 | VFEGREDSI |
| 849 | EFLWGPRAL |
| 850 | VAELVHFLL |
| 851 | IFSKASSSL |
| 852 | AELVHFLLL |

In some embodiments, the donor cell source is HLA-A*26, and the MAGE-A3 targeted T-cell subpopulation is primed and expanded with one or more MAGE-A3-derived peptides selected from Table 86 (Seq. ID. Nos. 853-862). In some embodiments, the donor cell source is HLA-A*26, and the MAGE-A3 targeted T-cell subpopulation is primed and expanded with MAGE-A3-derived peptides selected from Table 86 (Seq. ID. Nos. 853-862). In some embodiments, the donor cell source is HLA-A*26, and the MAGE-A3 targeted T-cell subpopulation is primed and expanded with MAGE-A3-derived peptides comprising the peptides of Table 86 (Seq. ID. Nos. 853-862). In some embodiments, the donor cell source is HLA-A*26, and the MAGE-A3 targeted T-cell subpopulation is primed and expanded with MAGE-A3-derived peptides comprising the peptides of Table 86 (Seq. ID. Nos. 853-862) and at least one additional set of peptides based on the donor cell source HLA-A profile, wherein the at least one additional set of peptides are selected from the peptides of Tables 81-85 and 87. In some embodiments, the MAGE-A3-derived peptides also include one or more sets of HLA-B and HLA-DR restricted peptides selected from Tables 88-100 (Seq. ID Nos. 873-1002).

TABLE 86
MAGEA3 HLA-A*26 Epitope Peptides

| SEQ ID NO. | Sequence |
|---|---|
| 853 | ELVHFLLLKY |
| 854 | EKIWEELSVL |
| 855 | EVFEGREDSI |
| 856 | EVTLGEVPAA |
| 857 | EVDPIGHLY |
| 858 | LVHFLLLKY |
| 859 | EVFEGREDS |
| 860 | KVAELVHFL |
| 861 | EPVTKAEML |
| 862 | SVVGNWQYF |

In some embodiments, the donor cell source is HLA-A*68:01, and the MAGE-A3 targeted T-cell subpopulation is primed and expanded with one or more MAGE-A3-derived peptides selected from Table 87 (Seq. ID. Nos. 863-872). In some embodiments, the donor cell source is HLA-A*68:01, and the MAGE-A3 targeted T-cell subpopulation is primed and expanded with MAGE-A3-derived peptides selected from Table 87 (Seq. ID. Nos. 863-872). In some embodiments, the donor cell source is HLA-A*68:01, and the MAGE-A3 targeted T-cell subpopulation is primed and expanded with MAGE-A3-derived peptides comprising the peptides of Table 87 (Seq. ID. Nos. 863-872). In some embodiments, the donor cell source is HLA-A*68:01, and the MAGE-A3 targeted T-cell subpopulation is primed and expanded with MAGE-A3-derived peptides comprising the peptides of Table 87 (Seq. ID. Nos. 863-872), and at least one additional set of peptides based on the donor cell source HLA-A profile, wherein the at least one additional set of peptides are selected from the peptides of Tables 81-86. In some embodiments, the MAGE-A3-derived peptides also include one or more sets of HLA-B and HLA-DR restricted peptides selected from Tables 88-100 (Seq. ID Nos. 873-1002).

TABLE 87
MAGEA3 HLA-A*68:01 Epitope Peptides

| SEQ ID NO. | Sequence |
|---|---|
| 863 | LLIIVLAIIAR |
| 864 | ELVHFLLLKYR |
| 865 | ELSVLEVFEGR |
| 866 | LIIVLAIIAR |
| 867 | ESEFQAALSR |
| 868 | IIVLAIIAR |
| 869 | ELVHFLLLK |
| 870 | IVLAIIARE |
| 871 | SVLEVFEGR |
| 872 | DSILGDPKK |

In some embodiments, the donor cell source is HLA-B*07:02, and the MAGE-A3 targeted T-cell subpopulation is primed and expanded with one or more MAGE-A3-derived peptides selected from Table 88 (Seq. ID. Nos. 873-882). In some embodiments, the donor cell source is HLA-B*07:02, and the MAGE-A3 targeted T-cell subpopulation is primed and expanded with MAGE-A3-derived peptides selected from Table 88 (Seq. ID. Nos. 873-882). In some embodiments, the donor cell source is HLA-B*07:02, and the MAGE-A3 targeted T-cell subpopulation is primed and expanded with MAGE-A3-derived peptides comprising the peptides of Table 88 (Seq. ID. Nos. 873-882). In some embodiments, the donor cell source is HLA-B*07:02, and the MAGE-A3 targeted T-cell subpopulation is primed and expanded with MAGE-A3-derived peptides comprising the peptides of Table 88 (Seq. ID. Nos. 873-882), and at least one additional set of peptides based on the donor cell source HLA-B profile, wherein the at least one additional set of peptides are selected from the peptides of Tables 89-94. In some embodiments, the MAGE-A3-derived peptides also include one or more sets of HLA-A and HLA-DR restricted peptides selected from Tables 81-87 and 95-100 (Seq. ID Nos. 803-872 and 943-1002).

TABLE 88
MAGEA3 HLA-B*07:02 Epitope Peptides

| SEQ ID NO. | Sequence |
|---|---|
| 873 | APEEKIWEEL |
| 874 | SPQGASSLPT |
| 875 | APATEEQEAA |
| 876 | DPIGHLYIFA |
| 877 | GPHISYPPL |
| 878 | LPTTMNYPL |
| 879 | EPVTKAEML |
| 880 | YPPLHEWVL |
| 881 | APATEEQEA |
| 882 | MPKAGLLII |

In some embodiments, the donor cell source is HLA-B*08, and the MAGE-A3 targeted T-cell subpopulation is primed and expanded with one or more MAGE-A3-derived peptides selected from Table 89 (Seq. ID. Nos. 883-892). In some embodiments, the donor cell source is HLA-B*08, and the MAGE-A3 targeted T-cell subpopulation is primed and expanded with MAGE-A3-derived peptides selected from Table 89 (Seq. ID. Nos. 883-892). In some embodiments, the donor cell source is HLA-B*08, and the MAGE-A3 targeted T-cell subpopulation is primed and expanded with MAGE-A3-derived peptides comprising the peptides of Table 89 (Seq. ID. Nos. 883-892). In some embodiments, the donor cell source is HLA-B*08, and the MAGE-A3 targeted T-cell subpopulation is primed and expanded with MAGE-A3-derived peptides comprising the peptides of Table 89 (Seq. ID. Nos. 883-892) and at least one additional set of peptides based on the donor cell source HLA-B profile, wherein the at least one additional set of peptides are selected from the peptides of Tables 88 and 90-94. In some embodiments, the MAGE-A3-derived peptides also include one or more sets of HLA-A and HLA-DR restricted peptides selected from Tables 81-87 and 95-100 (Seq. ID Nos. 803-872 and 943-1002).

TABLE 89
MAGEA3 HLA-B*08 Epitope Peptides

| SEQ ID NO. | Sequence |
|---|---|
| 883 | ALSRKVAEL |
| 884 | EPVTKAEML |
| 885 | GLEARGEAL |
| 886 | LLKYRAREP |
| 887 | QIMPKAGLL |
| 888 | EARGEALGL |
| 889 | MPKAGLLII |
| 890 | LLKYRARE |
| 891 | QIMPKAGL |
| 892 | EEKIWEEL |

In some embodiments, the donor cell source is HLA-B*15:01, and the MAGE-A3 targeted T-cell subpopulation is primed and expanded with one or more MAGE-A3-derived peptides selected from Table 90 (Seq. ID. Nos. 893-902). In some embodiments, the donor cell source is HLA-B*15:01, and the MAGE-A3 targeted T-cell subpopulation is primed and expanded with MAGE-A3-derived peptides selected from Table 90 (Seq. ID. Nos. 893-902). In some embodiments, the donor cell source is HLA-B*15:01, and the MAGE-A3 targeted T-cell subpopulation is primed and expanded with MAGE-A3-derived peptides comprising the peptides of Table 90 (Seq. ID. Nos. 893-902). In some embodiments, the donor cell source is HLA-B*15:01, and the MAGE-A3 targeted T-cell subpopulation is primed and expanded with MAGE-A3-derived peptides comprising the peptides of Table 90 (Seq. ID. Nos. 893-902) and at least one additional set of peptides based on the donor cell source HLA-B profile, wherein the at least one additional set of peptides are selected from the peptides of Tables 88-89 and 91-94. In some embodiments, the MAGE-A3-derived peptides also include one or more sets of HLA-A and HLA-DR restricted peptides selected from Tables 81-87 and 95-100 (Seq. ID Nos. 803-872 and 943-1002).

TABLE 90
MAGEA3 HLA-B*15:01 (B62) Epitope Peptides

| SEQ ID NO. | Sequence |
|---|---|
| 893 | NQEEEGPSTF |
| 894 | ELVHFLLLKY |
| 895 | QVPGSDPACY |
| 896 | SVVGNWQYFF |
| 897 | TQHFVQENY |
| 898 | LVHFLLLKY |
| 899 | FVQENYLEY |
| 900 | WQYFFPVIF |
| 901 | EVDPIGHLY |
| 902 | VVGNWQYFF |

In some embodiments, the donor cell source is HLA-B*18, and the MAGE-A3 targeted T-cell subpopulation is primed and expanded with one or more MAGE-A3-derived peptides selected from Table 91 (Seq. ID. Nos. 903-912). In some embodiments, the donor cell source is HLA-B*18, and the MAGE-A3 targeted T-cell subpopulation is primed and expanded with MAGE-A3-derived peptides selected from Table 91 (Seq. ID. Nos. 903-912). In some embodiments, the donor cell source is HLA-B*18, and the MAGE-A3 targeted T-cell subpopulation is primed and expanded with MAGE-A3-derived peptides comprising the peptides of Table 91 (Seq. ID. Nos. 903-912). In some embodiments, the donor cell source is HLA-B*18, and the MAGE-A3 targeted T-cell subpopulation is primed and expanded with MAGE-A3-derived peptides comprising the peptides of Table 91 (Seq. ID. Nos. 903-912) and at least one additional set of peptides based on the donor cell source HLA-B profile, wherein the at least one additional set of peptides are selected from the peptides of Tables 88-90 and 92-94. In some embodiments, the MAGE-A3-derived peptides also include one or more sets of HLA-A and HLA-DR restricted peptides selected from Tables 81-87 and 95-100 (Seq. ID Nos. 803-872 and 943-1002).

TABLE 91
MAGEA3 HLA-B*18 Epitope Peptides

| SEQ ID NO. | Sequence |
|---|---|
| 903 | EELSVLEVF |
| 904 | QEEEGPSTF |
| 905 | LESEFQAAL |
| 906 | PEEKIWEEL |
| 907 | AELVHFLLL |
| 908 | VETSYVKVL |
| 909 | EEEGPSTF |
| 910 | EEKIWEEL |
| 911 | AELVHFLL |
| 912 | LEARGEAL |

In some embodiments, the donor cell source is HLA-B*27:05, and the MAGE-A3 targeted T-cell subpopulation is primed and expanded with one or more MAGE-A3-derived peptides selected from Table 92 (Seq. ID. Nos. 913-922). In some embodiments, the donor cell source is HLA-B*27:05, and the MAGE-A3 targeted T-cell subpopulation is primed and expanded with MAGE-A3-derived peptides selected from Table 92 (Seq. ID. Nos. 913-922). In some embodiments, the donor cell source is HLA-B*27:05, and the MAGE-A3 targeted T-cell subpopulation is primed and expanded with MAGE-A3-derived peptides comprising the peptides of Table 92 (Seq. ID. Nos. 913-922). In some embodiments, the donor cell source is HLA-B*27:05, and the MAGE-A3 targeted T-cell subpopulation is primed and expanded with MAGE-A3-derived peptides comprising the peptides of Table 92 (Seq. ID. Nos. 913-922) and at least one additional set of peptides based on the donor cell source HLA-B profile, wherein the at least one additional set of peptides are selected from the peptides of Tables 88-91 and 93-94. In some embodiments, the MAGE-A3-derived peptides also include one or more sets of HLA-A and HLA-DR restricted peptides selected from Tables 81-87 and 95-100 (Seq. ID Nos. 803-872 and 943-1002).

TABLE 92
MAGEA3 HLA-B*27:05 Epitope Peptides

| SEQ ID NO. | Sequence |
|---|---|
| 913 | AREPVTKAEM |
| 914 | SRKVAELVHF |
| 915 | SEFQAALSRK |
| 916 | RALVETSYVK |
| 917 | YRAREPVTK |
| 918 | PRALVETSY |
| 919 | SRKVAELVH |
| 920 | YFFPVIFSK |
| 921 | KAGLLIIVL |
| 922 | DSILGDPKK |

In some embodiments, the donor cell source is HLA-B*35:01, and the MAGE-A3 targeted T-cell subpopulation is primed and expanded with one or more MAGE-A3-derived peptides selected from Table 93 (Seq. ID. Nos. 923-932). In some embodiments, the donor cell source is HLA-B*35:01, and the MAGE-A3 targeted T-cell subpopulation is primed and expanded with MAGE-A3-derived peptides selected from Table 93 (Seq. ID. Nos. 923-932). In some embodiments, the donor cell source is HLA-B*35:01, and the MAGE-A3 targeted T-cell subpopulation is primed and expanded with MAGE-A3-derived peptides comprising the peptides of Table 93 (Seq. ID. Nos. 923-932). In some embodiments, the donor cell source is HLA-B*35:01, and the MAGE-A3 targeted T-cell subpopulation is primed and expanded with MAGE-A3-derived peptides comprising the peptides of Table 93 (Seq. ID. Nos. 923-932) and at least one additional set of peptides based on the donor cell source HLA-B profile, wherein the at least one additional set of peptides are selected from the peptides of Tables 88-92 and 94. In some embodiments, the MAGE-A3-derived peptides also include one or more sets of HLA-A and HLA-DR restricted peptides selected from Tables 81-87 and 95-100 (Seq. ID Nos. 803-872 and 943-1002).

TABLE 93
MAGEA3 HLA-B*35:01 Epitope Peptides

| SEQ ID NO. | Sequence |
|---|---|
| 923 | APEEKIWEEL |
| 924 | GPRALVETSY |
| 925 | DPKKLLTQHF |
| 926 | EPVTKAEML |
| 927 | LPTTMNYPL |
| 928 | VPGSDPACY |
| 929 | YPPLHEWVL |
| 930 | GPHISYPPL |
| 931 | DPIGHLYIF |
| 932 | MPKAGLLII |

In some embodiments, the donor cell source is HLA-B*58:02, and the MAGE-A3 targeted T-cell subpopulation is primed and expanded with one or more MAGE-A3-derived peptides selected from Table 94 (Seq. ID. Nos. 933-942). In some embodiments, the donor cell source is HLA-B*58:02, and the MAGE-A3 targeted T-cell subpopulation is primed and expanded with MAGE-A3-derived peptides selected from Table 94 (Seq. ID. Nos. 933-942). In some embodiments, the donor cell source is HLA-B*58:02, and the MAGE-A3 targeted T-cell subpopulation is primed and expanded with MAGE-A3-derived peptides comprising the peptides of Table 94 (Seq. ID. Nos. 933-942). In some embodiments, the donor cell source is HLA-B*58:02, and the MAGE-A3 targeted T-cell subpopulation is primed and expanded with MAGE-A3-derived peptides comprising the peptides of Table 94 (Seq. ID. Nos. 933-942) and at least one additional set of peptides based on the donor cell source HLA-B profile, wherein the at least one additional set of peptides are selected from the peptides of Tables 88-93. In some embodiments, the MAGE-A3-derived peptides also include one or more sets of HLA-A and HLA-DR restricted peptides selected from Tables 81-87 and 95-100 (Seq. ID Nos. 803-872 and 943-1002).

TABLE 94
MAGEA3 HLA-B*58:02 Epitope Peptides

| SEQ ID NO. | Sequence |
|---|---|
| 933 | KVAELVHFLL |
| 934 | KASSSLQLVF |
| 935 | SSSTLVEVTL |
| 936 | FSKASSSLQL |
| 937 | KAGLLIIVL |
| 938 | KVAELVHFL |
| 939 | SSTLVEVTL |
| 940 | SSLQLVFGI |
| 941 | KVLHHMVKI |
| 942 | SSLPTTMNY |

In some embodiments, the donor cell source is HLA-DRB1*0101, and the MAGE-A3 targeted T-cell subpopulation is primed and expanded with one or more MAGE-A3-derived peptides selected from Table 95 (Seq. ID. Nos. 943-952). In some embodiments, the donor cell source is HLA-DRB1*0101, and the MAGE-A3 targeted T-cell subpopulation is primed and expanded with MAGE-A3-derived peptides selected from Table 95 (Seq. ID. Nos. 943-952). In some embodiments, the donor cell source is HLA-DRB1*0101, and the MAGE-A3 targeted T-cell subpopulation is primed and expanded with MAGE-A3-derived peptides comprising the peptides of Table 95 (Seq. ID. Nos. 943-952). In some embodiments, the donor cell source is HLA-DRB1*0101, and the MAGE-A3 targeted T-cell subpopulation is primed and expanded with MAGE-A3-derived peptides comprising the peptides of Table 95 (Seq. ID. Nos. 943-952) and at least one additional set of peptides based on the donor cell source HLA-DR profile, wherein the at least one additional set of peptides are selected from the peptides of Tables 96-100. In some embodiments, the MAGE-A3-derived peptides also include one or more sets of HLA-A and HLA-B restricted peptides selected from Tables 81-94 (Seq. ID Nos. 803-942).

TABLE 95
MAGEA3 HLA-DRB1*0101 Epitope Peptides

| SEQ ID NO. | Sequence |
|---|---|
| 943 | PACYEFLWGPRALVE |
| 944 | YLEYRQVPGSDPACY |
| 945 | AGLLIIVLAIIAREG |
| 946 | GEALGLVGAQAPATE |
| 947 | QYFFPVIFSKASSSL |
| 948 | SSSLQLVFGIELMEV |
| 949 | EVTLGEVPAAESPDP |
| 950 | HHMVKISGGPHISYP |
| 951 | HFLLLKYRAREPVTK |
| 952 | ETSYVKVLHHMVKIS |

In some embodiments, the donor cell source is HLA-DRB1*0301, and the MAGE-A3 targeted T-cell subpopulation is primed and expanded with one or more MAGE-A3-derived peptides selected from Table 96 (Seq. ID. Nos. 953-962). In some embodiments, the donor cell source is HLA-DRB1*0301, and the MAGE-A3 targeted T-cell subpopulation is primed and expanded with MAGE-A3-derived peptides selected from Table 96 (Seq. ID. Nos. 953-962). In some embodiments, the donor cell source is HLA-DRB1*0301, and the MAGE-A3 targeted T-cell subpopulation is primed and expanded with MAGE-A3-derived peptides comprising the peptides of Table 96 (Seq. ID. Nos. 953-962). In some embodiments, the donor cell source is HLA-DRB1*0301, and the MAGE-A3 targeted T-cell subpopulation is primed and expanded with MAGE-A3-derived peptides comprising the peptides of Table 96 (Seq. ID. Nos. 953-962) and at least one additional set of peptides based on the donor cell source HLA-DR profile, wherein the at least one additional set of peptides are selected from the peptides of Tables 95 and 97-100. In some embodiments, the MAGE-A3-derived peptides also include one or more sets of HLA-A and HLA-B restricted peptides selected from Tables 81-94 (Seq. ID Nos. 803-942).

TABLE 96
MAGEA3 HLA-DRB1*0301 (DR17) Epitope Peptides

| SEQ ID NO. | Sequence |
|---|---|
| 953 | EDSILGDPKKLLTQH |
| 954 | IELMEVDPIGHLYIF |
| 955 | YDGLLGDNQIMPKAG |
| 956 | FPDLESEFQAALSRK |
| 957 | GPSTFPDLESEFQAA |
| 958 | LGSVVGNWQYFFPVI |
| 959 | ASSLPTTMNYPLWSQ |
| 960 | VAELVHFLLLKYRAR |
| 961 | CLGLSYDGLLGDNQI |
| 962 | SRKVAELVHFLLLKY |

In some embodiments, the donor cell source is HLA-DRB1*0401, and the MAGE-A3 targeted T-cell subpopulation is primed and expanded with one or more MAGE-A3-derived peptides selected from Table 97 (Seq. ID. Nos. 963-972). In some embodiments, the donor cell source is HLA-DRB1*0401, and the MAGE-A3 targeted T-cell subpopulation is primed and expanded with MAGE-A3-derived peptides selected from Table 97 (Seq. ID. Nos. 963-972). In some embodiments, the donor cell source is HLA-DRB1*0401, and the MAGE-A3 targeted T-cell subpopulation is primed and expanded with MAGE-A3-derived peptides comprising the peptides of Table 97 (Seq. ID. Nos. 963-972). In some embodiments, the donor cell source is HLA-DRB1*0401, and the MAGE-A3 targeted T-cell subpopulation is primed and expanded with MAGE-A3-derived peptides comprising the peptides of Table 97 (Seq. ID. Nos. 963-972) and at least one additional set of peptides based on the donor cell source HLA-DR profile, wherein the at least one additional set of peptides are selected from the peptides of Tables 95-96 and 98-100. In some embodiments, the MAGE-A3-derived peptides also include one or more sets of HLA-A and HLA-B restricted peptides selected from Tables 81-94 (Seq. ID Nos. 803-942).

TABLE 97
MAGEA3 HLA-DRB1*0401 (DR4Dw4) Epitope Peptides

| SEQ ID NO. | Sequence |
|---|---|
| 963 | PSTFPDLESEFQAAL |
| 964 | ESEFQAALSRKVAEL |
| 965 | QYFFPVIFSKASSSL |
| 966 | PVIFSKASSSLQLVF |
| 967 | ETSYVKVLHHMVKIS |
| 968 | FPDLESEFQAALSRK |
| 969 | SRKVAELVHFLLLKY |
| 970 | LMEVDPIGHLYIFAT |
| 971 | TSYVKVLHHMVKISG |
| 972 | WQYFFPVIFSKASSS |

In some embodiments, the donor cell source is HLA-DRB1*0701, and the MAGE-A3 targeted T-cell subpopulation is primed and expanded with one or more MAGE-A3-derived peptides selected from Table 98 (Seq. ID. Nos. 973-982). In some embodiments, the donor cell source is HLA-DRB1*0701, and the MAGE-A3 targeted T-cell subpopulation is primed and expanded with MAGE-A3-derived peptides selected from Table 98 (Seq. ID. Nos. 973-982). In some embodiments, the donor cell source is HLA-DRB1*0701, and the MAGE-A3 targeted T-cell subpopulation is primed and expanded with MAGE-A3-derived peptides comprising the peptides of Table 98 (Seq. ID. Nos. 973-982). In some embodiments, the donor cell source is HLA-DRB1*0701, and the MAGE-A3 targeted T-cell subpopulation is primed and expanded with MAGE-A3-derived peptides comprising the peptides of Table 98 (Seq. ID. Nos. 973-982) and at least one additional set of peptides based on the donor cell source HLA-DR profile, wherein the at least one additional set of peptides are selected from the peptides of Tables 95-97 and 99-100. In some embodiments, the MAGE-A3-derived peptides also include one or more sets of HLA-A and HLA-B restricted peptides selected from Tables 81-94 (Seq. ID Nos. 803-942).

TABLE 98
MAGEA3 HLA-DRB1*0701 Epitope Peptides

| SEQ ID NO. | Sequence |
|---|---|
| 973 | ESEFQAALSRKVAEL |
| 974 | ASSLPTTMNYPLWSQ |
| 975 | ATCLGLSYDGLLGDN |
| 976 | QYFFPVIFSKASSSL |
| 977 | FPVIFSKASSSLQLV |
| 978 | PVIFSKASSSLQLVF |
| 979 | GHLYIFATCLGLSYD |
| 980 | LEVFEGREDSILGDP |
| 981 | PRALVETSYVKVLHH |
| 982 | HISYPPLHEWVLREG |

In some embodiments, the donor cell source is HLA-DRB1*1101, and the MAGE-A3 targeted T-cell subpopulation is primed and expanded with one or more MAGE-A3-derived peptides selected from Table 99 (Seq. ID. Nos. 983-992). In some embodiments, the donor cell source is HLA-DRB1*1101, and the MAGE-A3 targeted T-cell subpopulation is primed and expanded with MAGE-A3-derived peptides selected from Table 99 (Seq. ID. Nos. 983-992). In some embodiments, the donor cell source is HLA-DRB1*1101, and the MAGE-A3 targeted T-cell subpopulation is primed and expanded with MAGE-A3-derived peptides comprising the peptides of Table 99 (Seq. ID. Nos. 983-992). In some embodiments, the donor cell source is HLA-DRB1*1101, and the MAGE-A3 targeted T-cell subpopulation is primed and expanded with MAGE-A3-derived peptides comprising the peptides of Table 99 (Seq. ID. Nos. 983-992) and at least one additional set of peptides based on the donor cell source HLA-DR profile, wherein the at least one additional set of peptides are selected from the peptides of Tables 95-98 and 100. In some embodiments, the MAGE-A3-derived peptides also include one or more sets of HLA-A and HLA-B restricted peptides selected from Tables 81-94 (Seq. ID Nos. 803-942).

TABLE 99
MAGEA3 HLA-DRB1*1101 Epitope Peptides

| SEQ ID NO. | Sequence |
|---|---|
| 983 | VKVLHHMVKISGGPH |
| 984 | WQYFFPVIFSKASSS |
| 985 | PACYEFLWGPRALVE |
| 986 | ETSYVKVLHHMVKIS |
| 987 | SRKVAELVHFLLLKY |
| 988 | ELVHFLLLKYRAREP |
| 989 | QYFFPVIFSKASSSL |
| 990 | YLEYRQVPGSDPACY |
| 991 | TSYVKVLHHMVKISG |
| 992 | SEFQAALSRKVAELV |

In some embodiments, the donor cell source is HLA-DRB1*1501, and the MAGE-A3 targeted T-cell subpopulation is primed and expanded with one or more MAGE-A3-derived peptides selected from Table 100 (Seq. ID. Nos. 993-1002). In some embodiments, the donor cell source is HLA-DRB1*1501, and the MAGE-A3 targeted T-cell subpopulation is primed and expanded with MAGE-A3-derived peptides selected from Table 100 (Seq. ID. Nos. 993-1002). In some embodiments, the donor cell source is HLA-DRB1*1501, and the MAGE-A3 targeted T-cell subpopulation is primed and expanded with MAGE-A3-derived peptides comprising the peptides of Table 100 (Seq. ID. Nos. 993-1002). In some embodiments, the donor cell source is HLA-DRB1*1501, and the MAGE-A3 targeted T-cell subpopulation is primed and expanded with MAGE-A3-derived peptides comprising the peptides of Table 100 (Seq. ID. Nos. 993-1002) and at least one additional set of peptides based on the donor cell source HLA-DR profile, wherein the at least one additional set of peptides are selected from the peptides of Tables 95-99. In some embodiments, the MAGE-A3-derived peptides also include one or more sets of HLA-A and HLA-B restricted peptides selected from Tables 81-94 (Seq. ID Nos. 803-942).

TABLE 100
MAGEA3 HLA-DRB1*1501 (DR2b) Epitope Peptides

| SEQ ID NO. | Sequence |
|---|---|
| 993 | GSVVGNWQYFFPVIF |
| 994 | HFLLLKYRAREPVTK |
| 995 | IGHLYIFATCLGLSY |
| 996 | VAELVHFLLLKYRAR |
| 997 | SSSLQLVFGIELMEV |
| 998 | GIELMEVDPIGHLYI |
| 999 | TCLGLSYDGLLGDNQ |
| 1000 | DNQIMPKAGLLIIVL |
| 1001 | AGLLIIVLAIIAREG |
| 1002 | LSVLEVFEGREDSIL |

MAGE A4 Antigenic Peptides

In some embodiments, the MUSTANG composition includes MAGE-A4 (Melanoma-associated antigen 4) specific T-cells. MAGE-A4 specific T-cells can be generated as described below using one or more antigenic peptides to MAGE-A4. In some embodiments, the MAGE-A4 specific T-cells are generated using one or more antigenic peptides to MAGE-A4, or a modified or heteroclitic peptide derived from a MAGE-A4 peptide. In some embodiments, MAGE-A4 specific T-cells are generated using a MAGE-A4 antigen library comprising a pool of peptides (for example 15mers) containing amino acid overlap (for example 11 amino acids of overlap) between each sequence formed by scanning the protein amino acid sequence SEQ. ID. No. 1003 (UniProt KB-P43358) for MAGE-A4:

MSSEQKSQHCKPEEGVEAQEEALGLVGAQAPTTEEQEAAVSSSSPLVPGT
LEEVPAAESAGPPQSPQGASALPTTISFTCWRQPNEGSSSQEEEGPSTSP
DAESLFREALSNKVDELAHFLLRKYRAKELVTKAEMLERVIKNYKRCFPV
IFGKASESLKMIFGIDVKEVDPASNTYTLVTCLGLSYDGLLGNNQIFPKT
GLLIIVLGTIAMEGDSASEEEIWEELGVMGVYDGREHTVYGEPRKLLTQD
WVQENYLEYRQVPGSNPARYEFLWGPRALAETSYVKVLEHVVRVNARVRI
AYPSLREAALLEEEEGV

Overlapping antigenic libraries are commercially available, for example, from JPT, for example, from JPT (Product Code: PM-MAGEA4 (Pep Mix™ Human (MAGE-A4)). In some embodiments, the MAGE-A4 specific T-cells are generated using a commercially available overlapping antigenic library made up of MAGE-A4 peptides.

In some embodiments, the MAGE-A4 specific T-cells are generated using one or more antigenic peptides to MAGE-A4, or a modified or heteroclitic peptide derived from a MAGE-A4 peptide. In some embodiments, the MAGE-A4 specific T-cells are generated with peptides that recognize class I MHC molecules. In some embodiments, the MAGE-A4 specific T-cells are generated with peptides that recognize class II MEW molecules. In some embodiments, the MAGE-A4 specific T-cells are generated with peptides that recognize both class I and class II MEW molecules.

In some embodiments, the MAGE-A4 peptides used to prime and expand a T-cell subpopulation includes specifically selected HLA-restricted peptides generated by determining the HLA profile of the donor source, and including peptides derived from MAGE-A4 that best match the donor's HLA. In some embodiments, the MAGE-A4 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting MAGE-A4 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 101-107, the HLA-B peptides are selected from the peptides of Tables 108-114, and the HLA-DR peptides are selected from the peptides of Tables 115-120. For example, if the donor cell source has an HLA profile that is HLA-A*01/*02:01; HLA-B*15:01/*18; and HLA-DRB1*0101/*0301, then the MAGE-A4 peptides used to prime and expand the MAGE-A4 specific T-cell subpopulation are restricted to the specific HLA profile, and may include the peptides identified in Table 101 (Seq. ID. Nos. 1004-1013) for HLA-A*01; Table 102 (Seq. ID. Nos. 1014-1023) for HLA-A*02:01; Table 110 (Seq. ID. Nos. 1093-1102) for HLA-B*15:01; Table 111 (Seq. ID. Nos. 1103-1112) for HLA-B*18; Table 115 (Seq. ID. Nos. 1143-1152) for HLA-DRB1*0101; and Table 116 (Seq. ID. Nos. 1153-1162) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the MAGE-A4 targeted T-cell subpopulation is primed and expanded with one or more MAGE-A4-derived peptides selected from Table 101 (Seq. ID. Nos. 1004-1013). In some embodiments, the donor cell source is HLA-A*01, and the MAGE-A4 targeted T-cell subpopulation is primed and expanded with MAGE-A4-derived peptides selected from Table 101 (Seq. ID. Nos. 1004-1013). In some embodiments, the donor cell source is HLA-A*01, and the MAGE-A4 targeted T-cell subpopulation is primed and expanded with MAGE-A4-derived peptides comprising the peptides of Table 101 (Seq. ID. Nos. 1004-1013). In some embodiments, the donor cell source is HLA-A*01, and the MAGE-A4 targeted T-cell subpopulation is primed and expanded with MAGE-A4-derived peptides comprising the peptides of Table 101 (Seq. ID. Nos. 1004-1013) and at least one additional set of peptides based on the donor cell source HLA-A profile, wherein the at least one additional set of peptides are selected from the peptides of Tables 102-107. In some embodiments, the MAGE-A4-derived peptides also include one or more sets of HLA-B and HLA-DR restricted peptides selected from Tables 108-120 (Seq. ID Nos. 1073-1202).

TABLE 101
MAGEA4 HLA-A*01 Epitope Peptides

| SEQ ID NO. | Sequence |
|---|---|
| 1004 | YTLVTCLGLSY |
| 1005 | VKEVDPASNTY |
| 1006 | IWEELGVMGVY |
| 1007 | QDWVQENYLEY |
| 1008 | VYDGREHTVY |
| 1009 | WEELGVMGVY |
| 1010 | LTQDWVQENY |
| 1011 | EVDPASNTY |
| 1012 | TQDWVQENY |
| 1013 | MLERVIKNY |

In some embodiments, the donor cell source is HLA-A*02:01, and the MAGE-A4 targeted T-cell subpopulation is primed and expanded with one or more MAGE-A4-derived peptides selected from Table 102 (Seq. ID. Nos. 1014-1023). In some embodiments, the donor cell source is HLA-A*02:01, and the MAGE-A4 targeted T-cell subpopulation is primed and expanded with MAGE-A4-derived peptides selected from Table 102 (Seq. ID. Nos. 1014-1023). In some embodiments, the donor cell source is HLA-A*02:01, and the MAGE-A4 targeted T-cell subpopulation is primed and expanded with MAGE-A4-derived peptides comprising the peptides of Table 102 (Seq. ID. Nos. 1014-1023). In some embodiments, the donor cell source is HLA-A*02:01, and the MAGE-A4 targeted T-cell subpopulation is primed and expanded with MAGE-A4-derived peptides comprising the peptides of Table 102 (Seq. ID. Nos. 1014-1023) and at least one additional set of peptides based on the donor cell source HLA-A profile, wherein the at least one additional set of peptides are selected from the peptides of Tables 101, and 103-107. In some embodiments, the MAGE-A4-derived peptides also include one or more sets of HLA-B and HLA-DR restricted peptides selected from Tables 108-120 (Seq. ID Nos. 1073-1202).

TABLE 102
MAGEA4 HLA-A*02:01 Epitope Peptides

| SEQ ID NO. | Sequence |
|---|---|
| 1014 | ALAETSYVKV |
| 1015 | GLLIIVLGTI |
| 1016 | MIFGIDVKEV |
| 1017 | PLVPGTLEEV |
| 1018 | VIFGKASESL |
| 1019 | ALSNKVDEL |
| 1020 | LLIIVLGTI |
| 1021 | ALLEEEEGV |
| 1022 | KVLEHVVRV |
| 1023 | QIFPKTGLL |

In some embodiments, the donor cell source is HLA-A*03, and the MAGE-A4 targeted T-cell subpopulation is primed and expanded with one or more MAGE-A4-derived peptides selected from Table 103 (Seq. ID. Nos. 1024-1033). In some embodiments, the donor cell source is HLA-A*03, and the MAGE-A4 targeted T-cell subpopulation is primed and expanded with MAGE-A4-derived peptides selected from Table 103 (Seq. ID. Nos. 1024-1033). In some embodiments, the donor cell source is HLA-A*03, and the MAGE-A4 targeted T-cell subpopulation is primed and expanded with MAGE-A4-derived peptides comprising the peptides of Table 103 (Seq. ID. Nos. 1024-1033). In some embodiments, the donor cell source is HLA-A*03, and the MAGE-A4 targeted T-cell subpopulation is primed and expanded with MAGE-A4-derived peptides comprising the peptides of Table 103 (Seq. ID. Nos. 1024-1033) and at least one additional set of peptides based on the donor cell source HLA-A profile, wherein the at least one additional set of peptides are selected from the peptides of Tables 101-102 and 104-107. In some embodiments, the MAGE-A4-derived peptides also include one or more sets of HLA-B and HLA-DR restricted peptides selected from Tables 108-120 (Seq. ID Nos. 1073-1202).

TABLE 103
MAGEA4 HLA-A*03 Epitope Peptides

| SEQ ID NO. | Sequence |
|---|---|
| 1024 | SLFREALSNK |
| 1025 | KYRAKELVTK |
| 1026 | RVRIAYPSLR |
| 1027 | TLVTCLGLSY |
| 1028 | QVPGSNPARY |
| 1029 | HVVRVNARVR |
| 1030 | ALAETSYVK |
| 1031 | FLLRKYRAK |
| 1032 | ALGLVGAQA |
| 1033 | ELAHFLLRK |

In some embodiments, the donor cell source is HLA-A*11:01, and the MAGE-A4 targeted T-cell subpopulation is primed and expanded with one or more MAGE-A4-derived peptides selected from Table 104 (Seq. ID. Nos. 1034-1043). In some embodiments, the donor cell source is HLA-A*11:01, and the MAGE-A4 targeted T-cell subpopulation is primed and expanded with MAGE-A4-derived peptides selected from Table 104 (Seq. ID. Nos. 1034-1043). In some embodiments, the donor cell source is HLA-A*11:01, and the MAGE-A4 targeted T-cell subpopulation is primed and expanded with MAGE-A4-derived peptides comprising the peptides of Table 104 (Seq. ID. Nos. 1034-1043). In some embodiments, the donor cell source is HLA-A*11:01, and the MAGE-A4 targeted T-cell subpopulation is primed and expanded with MAGE-A4-derived peptides comprising the peptides of Table 104 (Seq. ID. Nos. 1034-1043), and at least one additional set of peptides based on the donor cell source HLA-A profile, wherein the at least one additional set of peptides are selected from the peptides of Tables 101-103 and 105-107. In some embodiments, the MAGE-A4-derived peptides also include one or more sets of HLA-B and HLA-DR restricted peptides selected from Tables 108-120 (Seq. ID Nos. 1073-1202).

TABLE 104
MAGEA4 HLA-A*11:01 Epitope Peptides

| SEQ ID NO. | Sequence |
|---|---|
| 1034 | TSPDAESLFR |
| 1035 | YVKVLEHVVR |
| 1036 | SSEQKSQHCK |
| 1037 | LVTKAEMLER |
| 1038 | RVRIAYPSLR |
| 1039 | VTKAEMLER |
| 1040 | ELAHFLLRK |
| 1041 | GVMGVYDGR |
| 1042 | TTISFTCWR |
| 1043 | ALAETSYVK |

In some embodiments, the donor cell source is HLA-A*24:02, and the MAGE-A4 targeted T-cell subpopulation is primed and expanded with one or more MAGE-A4-derived peptides selected from Table 105 (Seq. ID. Nos. 1044-1052). In some embodiments, the donor cell source is HLA-A*24:02, and the MAGE-A4 targeted T-cell subpopulation is primed and expanded with MAGE-A4-derived peptides selected from Table 105 (Seq. ID. Nos. 1044-1052). In some embodiments, the donor cell source is HLA-A*24:02, and the MAGE-A4 targeted T-cell subpopulation is primed and expanded with MAGE-A4-derived peptides comprising the peptides of Table 105 (Seq. ID. Nos. 1044-1052). In some embodiments, the donor cell source is HLA-A*24:02, and the MAGE-A4 targeted T-cell subpopulation is primed and expanded with MAGE-A4-derived peptides comprising the peptides of Table 105 (Seq. ID. Nos. 1044-1052), and at least one additional set of peptides based on the donor cell source HLA-A profile, wherein the at least one additional set of peptides are selected from the peptides of Tables 101-104 and 106-107. In some embodiments, the MAGE-A4-derived peptides also include one or more sets of HLA-B and HLA-DR restricted peptides selected from Tables 108-120 (Seq. ID Nos. 1073-1202).

TABLE 105
MAGEA4 HLA-A*24:02 Epitope Peptides

| SEQ ID NO. | Sequence |
|---|---|
| 1044 | AYPSLREAAL |
| 1045 | TYTLVTCLGL |
| 1046 | NYKRCFPVIF |
| 1047 | IFPKTGLLII |
| 1048 | KVDELAHFLL |
| 1049 | VYGEPRKLL |
| 1050 | NYKRCFPVI |
| 1051 | EFLWGPRAL |
| 1052 | IFGKASESL |

In some embodiments, the donor cell source is HLA-A*26, and the MAGE-A4 targeted T-cell subpopulation is primed and expanded with one or more MAGE-A4-derived peptides selected from Table 106 (Seq. ID. Nos. 1053-1062). In some embodiments, the donor cell source is HLA-A*26, and the MAGE-A4 targeted T-cell subpopulation is primed and expanded with MAGE-A4-derived peptides selected from Table 106 (Seq. ID. Nos. 1053-1062). In some embodiments, the donor cell source is HLA-A*26, and the MAGE-A4 targeted T-cell subpopulation is primed and expanded with MAGE-A4-derived peptides comprising the peptides of Table 106 (Seq. ID. Nos. 1053-1062). In some embodiments, the donor cell source is HLA-A*26, and the MAGE-A4 targeted T-cell subpopulation is primed and expanded with MAGE-A4-derived peptides comprising the peptides of Table 106 (Seq. ID. Nos. 1053-1062) and at least one additional set of peptides based on the donor cell source HLA-A profile, wherein the at least one additional set of peptides are selected from the peptides of Tables 101-105 and 107. In some embodiments, the MAGE-A4-derived peptides also include one or more sets of HLA-B and HLA-DR restricted peptides selected from Tables 108-120 (Seq. ID Nos. 1073-1202).

TABLE 106
MAGEA4 HLA-A*26 Epitope Peptides

| SEQ ID NO. | Sequence |
|---|---|
| 1053 | EMLERVIKNY |
| 1054 | EGVEAQEEAL |
| 1055 | ELAHFLLRKY |
| 1056 | EALSNKVDEL |
| 1057 | DWVQENYLEY |
| 1058 | ETSYVKVLEH |
| 1059 | EVDPASNTY |
| 1060 | LVTCLGLSY |
| 1061 | ELVTKAEML |
| 1062 | WVQENYLEY |

In some embodiments, the donor cell source is HLA-A*68:01, and the MAGE-A4 targeted T-cell subpopulation is primed and expanded with one or more MAGE-A4-derived peptides selected from Table 107 (Seq. ID. Nos. 1063-1072). In some embodiments, the donor cell source is HLA-A*68:01, and the MAGE-A4 targeted T-cell subpopulation is primed and expanded with MAGE-A4-derived peptides selected from Table 107 (Seq. ID. Nos. 1063-1072). In some embodiments, the donor cell source is HLA-A*68:01, and the MAGE-A4 targeted T-cell subpopulation is primed and expanded with MAGE-A4-derived peptides comprising the peptides of Table 107 (Seq. ID. Nos. 1063-1072). In some embodiments, the donor cell source is HLA-A*68:01, and the MAGE-A4 targeted T-cell subpopulation is primed and expanded with MAGE-A4-derived peptides comprising the peptides of Table 107 (Seq. ID. Nos. 1063-1072), and at least one additional set of peptides based on the donor cell source HLA-A profile, wherein the at least one additional set of peptides are selected from the peptides of Tables 101-106. In some embodiments, the MAGE-A4-derived peptides also include one or more sets of HLA-B and HLA-DR restricted peptides selected from Tables 108-120 (Seq. ID Nos. 1073-1202).

TABLE 107
MAGEA4 HLA-A*68:01 Epitope Peptides

| SEQ ID NO. | Sequence |
|---|---|
| 1063 | ELAHFLLRKYR |
| 1064 | STSPDAESLFR |
| 1065 | ELVTKAEMLER |
| 1066 | YVKVLEHVVR |
| 1067 | PTTISFTCWR |
| 1068 | LVTKAEMLER |
| 1069 | ELAHFLLRK |
| 1070 | TTISFTCWR |
| 1071 | GVMGVYDGR |
| 1072 | QVPGSNPAR |

In some embodiments, the donor cell source is HLA-B*07:02, and the MAGE-A4 targeted T-cell subpopulation is primed and expanded with one or more MAGE-A4-derived peptides selected from Table 108 (Seq. ID. Nos. 1073-1082). In some embodiments, the donor cell source is HLA-B*07:02, and the MAGE-A4 targeted T-cell subpopulation is primed and expanded with MAGE-A4-derived peptides selected from Table 108 (Seq. ID. Nos. 1073-1082). In some embodiments, the donor cell source is HLA-B*07:02, and the MAGE-A4 targeted T-cell subpopulation is primed and expanded with MAGE-A4-derived peptides comprising the peptides of Table 108 (Seq. ID. Nos. 1073-1082). In some embodiments, the donor cell source is HLA-B*07:02, and the MAGE-A4 targeted T-cell subpopulation is primed and expanded with MAGE-A4-derived peptides comprising the peptides of Table 108 (Seq. ID. Nos. 1073-1082), and at least one additional set of peptides based on the donor cell source HLA-B profile, wherein the at least one additional set of peptides are selected from the peptides of Tables 109-114. In some embodiments, the MAGE-A4-derived peptides also include one or more sets of HLA-A and HLA-DR restricted peptides selected from Tables 101-107 and 115-120 (Seq. ID Nos. 1004-1072 and 1143-1202).

TABLE 108
MAGEA4 HLA-B*07:02 Epitope Peptides

| SEQ ID NO. | Sequence |
|---|---|
| 1073 | YPSLREAALL |
| 1074 | SPQGASALPT |
| 1075 | VPGTLEEVPA |
| 1076 | APTTEEQEAA |
| 1077 | DPASNTYTLV |
| 1078 | PPQSPQGASA |
| 1079 | YPSLREAAL |
| 1080 | DPASNTYTL |
| 1081 | APTTEEQEA |
| 1082 | FPKTGLLII |

In some embodiments, the donor cell source is HLA-B*08, and the MAGE-A4 targeted T-cell subpopulation is primed and expanded with one or more MAGE-A4-derived peptides selected from Table 109 (Seq. ID. Nos. 1083-1092). In some embodiments, the donor cell source is HLA-B*08, and the MAGE-A4 targeted T-cell subpopulation is primed and expanded with MAGE-A4-derived peptides selected from Table 109 (Seq. ID. Nos. 1083-1092). In some embodiments, the donor cell source is HLA-B*08, and the MAGE-A4 targeted T-cell subpopulation is primed and expanded with MAGE-A4-derived peptides comprising the peptides of Table 109 (Seq. ID. Nos. 1083-1092). In some embodiments, the donor cell source is HLA-B*08, and the MAGE-A4 targeted T-cell subpopulation is primed and expanded with MAGE-A4-derived peptides comprising the peptides of Table 109 (Seq. ID. Nos. 1083-1092) and at least one additional set of peptides based on the donor cell source HLA-B profile, wherein the at least one additional set of peptides are selected from the peptides of Tables 108 and 110-114. In some embodiments, the MAGE-A4-derived peptides also include one or more sets of HLA-A and HLA-DR restricted peptides selected from Tables 101-107 and 115-120 (Seq. ID Nos. 1004-1072 and 1143-1202).

TABLE 109
MAGEA4 HLA-B*08 Epitope Peptides

| SEQ ID NO. | Sequence |
|---|---|
| 1083 | LRKYRAKEL |
| 1084 | ALSNKVDEL |
| 1085 | ELVTKAEML |
| 1086 | YPSLREAAL |
| 1087 | QIFPKTGLL |
| 1088 | VIKNYKRCF |
| 1089 | SLREAALL |
| 1090 | SLKMIFGI |
| 1091 | QIFPKTGL |
| 1092 | FPKTGLLI |

In some embodiments, the donor cell source is HLA-B*15:01, and the MAGE-A4 targeted T-cell subpopulation is primed and expanded with one or more MAGE-A4-derived peptides selected from Table 110 (Seq. ID. Nos. 1093-1102). In some embodiments, the donor cell source is HLA-B*15:01, and the MAGE-A4 targeted T-cell subpopulation is primed and expanded with MAGE-A4-derived peptides selected from Table 110 (Seq. ID. Nos. 1093-1102). In some embodiments, the donor cell source is HLA-B*15:01, and the MAGE-A4 targeted T-cell subpopulation is primed and expanded with MAGE-A4-derived peptides comprising the peptides of Table 110 (Seq. ID. Nos. 1093-1102). In some embodiments, the donor cell source is HLA-B*15:01, and the MAGE-A4 targeted T-cell subpopulation is primed and expanded with MAGE-A4-derived peptides comprising the peptides of Table 110 (Seq. ID. Nos. 1093-1102) and at least one additional set of peptides based on the donor cell source HLA-B profile, wherein the at least one additional set of peptides are selected from the peptides of Tables 108-109 and 111-114. In some embodiments, the MAGE-A4-derived peptides also include one or more sets of HLA-A and HLA-DR restricted peptides selected from Tables 101-107 and 115-120 (Seq. ID Nos. 1004-1072 and 1143-1202).

TABLE 110
MAGEA4 HLA-B*15:01 (B62) Epitope Peptides

| SEQ ID NO. | Sequence |
|---|---|
| 1093 | TLVTCLGLSY |
| 1094 | RVNARVRIAY |
| 1095 | ELAHFLLRKY |
| 1096 | QVPGSNPARY |
| 1097 | RVIKNYKRCF |
| 1098 | MLERVIKNY |
| 1099 | TQDWVQENY |
| 1100 | LVTCLGLSY |
| 1101 | WVQENYLEY |
| 1102 | EVDPASNTY |

In some embodiments, the donor cell source is HLA-B*18, and the MAGE-A4 targeted T-cell subpopulation is primed and expanded with one or more MAGE-A4-derived peptides selected from Table 111 (Seq. ID. Nos. 1103-1112). In some embodiments, the donor cell source is HLA-B*18, and the MAGE-A4 targeted T-cell subpopulation is primed and expanded with MAGE-A4-derived peptides selected from Table 111 (Seq. ID. Nos. 1103-1112). In some embodiments, the donor cell source is HLA-B*18, and the MAGE-A4 targeted T-cell subpopulation is primed and expanded with MAGE-A4-derived peptides comprising the peptides of Table 111 (Seq. ID. Nos. 1103-1112). In some embodiments, the donor cell source is HLA-B*18, and the MAGE-A4 targeted T-cell subpopulation is primed and expanded with MAGE-A4-derived peptides comprising the peptides of Table 111 (Seq. ID. Nos. 1103-1112) and at least one additional set of peptides based on the donor cell source HLA-B profile, wherein the at least one additional set of peptides are selected from the peptides of Tables 108-110 and 112-114. In some embodiments, the MAGE-A4-derived peptides also include one or more sets of HLA-A and HLA-DR restricted peptides selected from Tables 101-107 and 115-120 (Seq. ID Nos. 1004-1072 and 1143-1202).

TABLE 111
MAGEA4 HLA-B*18 Epitope Peptides

| SEQ ID NO. | Sequence |
|---|---|
| 1103 | AESLFREAL |
| 1104 | SEEEIWEEL |
| 1105 | EELGVMGVY |
| 1106 | AETSYVKVL |
| 1107 | DELAHFLL |
| 1108 | EEEIWEEL |
| 1109 | LERVIKNY |
| 1110 | SESLKMIF |
| 1111 | VEAQEEAL |
| 1112 | DGREHTVY |

In some embodiments, the donor cell source is HLA-B*27:05, and the MAGE-A4 targeted T-cell subpopulation is primed and expanded with one or more MAGE-A4-derived peptides selected from Table 112 (Seq. ID. Nos. 1113-1122). In some embodiments, the donor cell source is HLA-B*27:05, and the MAGE-A4 targeted T-cell subpopulation is primed and expanded with MAGE-A4-derived peptides selected from Table 112 (Seq. ID. Nos. 1113-1122). In some embodiments, the donor cell source is HLA-B*27:05, and the MAGE-A4 targeted T-cell subpopulation is primed and expanded with MAGE-A4-derived peptides comprising the peptides of Table 112 (Seq. ID. Nos. 1113-1122). In some embodiments, the donor cell source is HLA-B*27:05, and the MAGE-A4 targeted T-cell subpopulation is primed and expanded with MAGE-A4-derived peptides comprising the peptides of Table 112 (Seq. ID. Nos. 1113-1122) and at least one additional set of peptides based on the donor cell source HLA-B profile, wherein the at least one additional set of peptides are selected from the peptides of Tables 108-111 and 113-114. In some embodiments, the MAGE-A4-derived peptides also include one or more sets of HLA-A and HLA-DR restricted peptides selected from Tables 101-107 and 115-120 (Seq. ID Nos. 1004-1072 and 1143-1202).

TABLE 112
MAGEA4 HLA-B*27:05 Epitope Peptides

| SEQ ID NO. | Sequence |
|---|---|
| 1113 | KRCFPVIFGK |
| 1114 | ARYEFLWGPR |
| 1115 | ARVRIAYPSL |
| 1116 | YRAKELVTK |
| 1117 | ERVIKNYKR |
| 1118 | VRIAYPSLR |
| 1119 | LRKYRAKEL |
| 1120 | RCFPVIFGK |
| 1121 | PRALAETSY |
| 1122 | KMIFGIDVK |

In some embodiments, the donor cell source is HLA-B*35:01, and the MAGE-A4 targeted T-cell subpopulation is primed and expanded with one or more MAGE-A4-derived peptides selected from Table 113 (Seq. ID. Nos. 1123-1132). In some embodiments, the donor cell source is HLA-B*35:01, and the MAGE-A4 targeted T-cell subpopulation is primed and expanded with MAGE-A4-derived peptides selected from Table 113 (Seq. ID. Nos. 1123-1132). In some embodiments, the donor cell source is HLA-B*35:01, and the MAGE-A4 targeted T-cell subpopulation is primed and expanded with MAGE-A4-derived peptides comprising the peptides of Table 113 (Seq. ID. Nos. 1123-1132). In some embodiments, the donor cell source is HLA-B*35:01, and the MAGE-A4 targeted T-cell subpopulation is primed and expanded with MAGE-A4-derived peptides comprising the peptides of Table 113 (Seq. ID. Nos. 1123-1132) and at least one additional set of peptides based on the donor cell source HLA-B profile, wherein the at least one additional set of peptides are selected from the peptides of Tables 108-112 and 114. In some embodiments, the MAGE-A4-derived peptides also include one or more sets of HLA-A and HLA-DR restricted peptides selected from Tables 101-107 and 115-120 (Seq. ID Nos. 1004-1072 and 1143-1202).

TABLE 113
MAGEA4 HLA-B*35:01 Epitope Peptides

| SEQ ID NO. | Sequence |
|---|---|
| 1123 | YPSLREAALL |
| 1124 | GPRALAETSY |
| 1125 | DPASNTYTL |
| 1126 | YPSLREAAL |
| 1127 | VPGSNPARY |
| 1128 | FPKTGLLII |
| 1129 | LPTTISFTC |
| 1130 | KVDELABFL |
| 1131 | MLERVIKNY |
| 1132 | LGLSYDGLL |

In some embodiments, the donor cell source is HLA-B*58:02, and the MAGE-A4 targeted T-cell subpopulation is primed and expanded with one or more MAGE-A4-derived peptides selected from Table 114 (Seq. ID. Nos. 1133-1142). In some embodiments, the donor cell source is HLA-B*58:02, and the MAGE-A4 targeted T-cell subpopulation is primed and expanded with MAGE-A4-derived peptides selected from Table 114 (Seq. ID. Nos. 1133-1142). In some embodiments, the donor cell source is HLA-B*58:02, and the MAGE-A4 targeted T-cell subpopulation is primed and expanded with MAGE-A4-derived peptides comprising the peptides of Table 114 (Seq. ID. Nos. 1133-1142). In some embodiments, the donor cell source is HLA-B*58:02, and the MAGE-A4 targeted T-cell subpopulation is primed and expanded with MAGE-A4-derived peptides comprising the peptides of Table 114 (Seq. ID. Nos. 1133-1142) and at least one additional set of peptides based on the donor cell source HLA-B profile, wherein the at least one additional set of peptides are selected from the peptides of Tables 108-113. In some embodiments, the MAGE-A4-derived peptides also include one or more sets of HLA-A and HLA-DR restricted peptides selected from Tables 101-107 and 115-120 (Seq. ID Nos. 1004-1072 and 1143-1202).

TABLE 114
MAGEA4 HLA-B*58:02 Epitope Peptides

| SEQ ID NO. | Sequence |
|---|---|
| 1133 | RVIKNYKRCF |
| 1134 | KASESLKMIF |
| 1135 | SSSPLVPGTL |
| 1136 | KAEMLERVI |
| 1137 | KTGLLIIVL |
| 1138 | KVDELAHFL |
| 1139 | KASESLKMI |
| 1140 | PSLREAALL |
| 1141 | SSPLVPGTL |
| 1142 | LAHFLLRKY |

In some embodiments, the donor cell source is HLA-DRB1*0101, and the MAGE-A4 targeted T-cell subpopulation is primed and expanded with one or more MAGE-A4-derived peptides selected from Table 115 (Seq. ID. Nos. 1143-1152). In some embodiments, the donor cell source is HLA-DRB1*0101, and the MAGE-A4 targeted T-cell subpopulation is primed and expanded with MAGE-A4-derived peptides selected from Table 115 (Seq. ID. Nos. 1143-1152). In some embodiments, the donor cell source is HLA-DRB1*0101, and the MAGE-A4 targeted T-cell subpopulation is primed and expanded with MAGE-A4-derived peptides comprising the peptides of Table 115 (Seq. ID. Nos. 1143-1152). In some embodiments, the donor cell source is HLA-DRB1*0101, and the MAGE-A4 targeted T-cell subpopulation is primed and expanded with MAGE-A4-derived peptides comprising the peptides of Table 115 (Seq. ID. Nos. 1143-1152) and at least one additional set of peptides based on the donor cell source HLA-DR profile, wherein the at least one additional set of peptides are selected from the peptides of Tables 116-120. In some embodiments, the MAGE-A4-derived peptides also include one or more sets of HLA-A and HLA-B restricted peptides selected from Tables 101-114 (Seq. ID Nos. 1004-1142).

TABLE 115
MAGEA4 HLA-DRB1*0101 Epitope Peptides

| SEQ ID NO. | Sequence |
|---|---|
| 1143 | PARYEFLWGPRALAE |
| 1144 | TGLLIIVLGTIAMEG |
| 1145 | YLEYRQVPGSNPARY |
| 1146 | KRCFPVIFGKASESL |
| 1147 | EEALGLVGAQAPTTE |
| 1148 | SESLKMIFGIDVKEV |
| 1149 | GLLIIVLGTIAMEGD |
| 1150 | PGTLEEVPAAESAGP |
| 1151 | HFLLRKYRAKELVTK |
| 1152 | EEIWEELGVMGVYDG |

In some embodiments, the donor cell source is HLA-DRB1*0301, and the MAGE-A4 targeted T-cell subpopulation is primed and expanded with one or more MAGE-A4-derived peptides selected from Table 116 (Seq. ID. Nos. 1153-1162). In some embodiments, the donor cell source is HLA-DRB1*0301, and the MAGE-A4 targeted T-cell subpopulation is primed and expanded with MAGE-A4-derived peptides selected from Table 116 (Seq. ID. Nos. 1153-1162). In some embodiments, the donor cell source is HLA-DRB1*0301, and the MAGE-A4 targeted T-cell subpopulation is primed and expanded with MAGE-A4-derived peptides comprising the peptides of Table 116 (Seq. ID. Nos. 1153-1162). In some embodiments, the donor cell source is HLA-DRB1*0301, and the MAGE-A4 targeted T-cell subpopulation is primed and expanded with MAGE-A4-derived peptides comprising the peptides of Table 116 (Seq. ID. Nos. 1153-1162). and at least one additional set of peptides based on the donor cell source HLA-DR profile, wherein the at least one additional set of peptides are selected from the peptides of Tables 115 and 117-120. In some embodiments, the MAGE-A4-derived peptides also include one or more sets of HLA-A and HLA-B restricted peptides selected from Tables 101-114 (Seq. ID Nos. 1004-1142).

TABLE 116
MAGEA4 HLA-DRB1*0301 (DR17) Epitope Peptides

| SEQ ID NO. | Sequence |
|---|---|
| 1153 | GPSTSPDAESLFREA |
| 1154 | EHTVYGEPRKLLTQD |
| 1155 | LERVIKNYKRCFPVI |
| 1156 | VVRVNARVRIAYPSL |
| 1157 | KMIFGIDVKEVDPAS |
| 1158 | KAEMLERVIKNYKRC |
| 1159 | CLGLSYDGLLGNNQI |
| 1160 | RKLLTQDWVQENYLE |
| 1161 | ALSNKVDELAHFLLR |
| 1162 | TYTLVTCLGLSYDGL |

In some embodiments, the donor cell source is HLA-DRB1*0401, and the MAGE-A4 targeted T-cell subpopulation is primed and expanded with one or more MAGE-A4-derived peptides selected from Table 117 (Seq. ID. Nos. 1163-1172). In some embodiments, the donor cell source is HLA-DRB1*0401, and the MAGE-A4 targeted T-cell subpopulation is primed and expanded with MAGE-A4-derived peptides selected from Table 117 (Seq. ID. Nos. 1163-1172). In some embodiments, the donor cell source is HLA-DRB1*0401, and the MAGE-A4 targeted T-cell subpopulation is primed and expanded with MAGE-A4-derived peptides comprising the peptides of Table 117 (Seq. ID. Nos. 1163-1172). In some embodiments, the donor cell source is HLA-DRB1*0401, and the MAGE-A4 targeted T-cell subpopulation is primed and expanded with MAGE-A4-derived peptides comprising the peptides of Table 117 (Seq. ID. Nos. 1163-1172) and at least one additional set of peptides based on the donor cell source HLA-DR profile, wherein the at least one additional set of peptides are selected from the peptides of Tables 115-116 and 118-120. In some embodiments, the MAGE-A4-derived peptides also include one or more sets of HLA-A and HLA-B restricted peptides selected from Tables 101-114 (Seq. ID Nos. 1004-1142).

TABLE 117
MAGEA4 HLA-DRB1*0401 (DR4Dw4) Epitope Peptides

| SEQ ID NO. | Sequence |
|---|---|
| 1163 | ESLFREALSNKVDEL |
| 1164 | SNKVDELAHFLLRKY |
| 1165 | VKEVDPASNTYTLVT |
| 1166 | GLLIIVLGTIAMEGD |
| 1167 | EHTVYGEPRKLLTQD |
| 1168 | VKVLEHVVRVNARVR |
| 1169 | KRCFPVIFGKASESL |
| 1170 | PVIFGKASESLKMIF |
| 1171 | SNTYTLVTCLGLSYD |
| 1172 | GLSYDGLLGNNQIFP |

In some embodiments, the donor cell source is HLA-DRB1*0701, and the MAGE-A4 targeted T-cell subpopulation is primed and expanded with one or more MAGE-A4-derived peptides selected from Table 118 (Seq. ID. Nos. 1173-1182). In some embodiments, the donor cell source is HLA-DRB1*0701, and the MAGE-A4 targeted T-cell subpopulation is primed and expanded with MAGE-A4-derived peptides selected from Table 118 (Seq. ID. Nos. 1173-1182). In some embodiments, the donor cell source is HLA-DRB1*0701, and the MAGE-A4 targeted T-cell subpopulation is primed and expanded with MAGE-A4-derived peptides comprising the peptides of Table 118 (Seq. ID. Nos. 1173-1182). In some embodiments, the donor cell source is HLA-DRB1*0701, and the MAGE-A4 targeted T-cell subpopulation is primed and expanded with MAGE-A4-derived peptides comprising the peptides of Table 118 (Seq. ID. Nos. 1173-1182) and at least one additional set of peptides based on the donor cell source HLA-DR profile, wherein the at least one additional set of peptides are selected from the peptides of Tables 115-117 and 119-120. In some embodiments, the MAGE-A4-derived peptides also include one or more sets of HLA-A and HLA-B restricted peptides selected from Tables 101-114 (Seq. ID Nos. 1004-1142).

TABLE 118
MAGEA4 HLA-DRB1*0701 Epitope Peptides

| SEQ ID NO. | Sequence |
|---|---|
| 1173 | ESLFREALSNKVDEL |
| 1174 | VTCLGLSYDGLLGNN |
| 1175 | NQIFPKTGLLIIVLG |
| 1176 | ASALPTTISFTCWRQ |
| 1177 | FPVIFGKASESLKMI |
| 1178 | GLLIIVLGTIAMEGD |
| 1179 | PRALAETSYVKVLEH |
| 1180 | ETSYVKVLEHVVRVN |
| 1181 | RIAYPSLREAALLEE |
| 1182 | EQEAAVSSSSPLVPG |

In some embodiments, the donor cell source is HLA-DRB1*1101, and the MAGE-A4 targeted T-cell subpopulation is primed and expanded with one or more MAGE-A4-derived peptides selected from Table 119 (Seq. ID. Nos. 1183-1192). In some embodiments, the donor cell source is HLA-DRB1*1101, and the MAGE-A4 targeted T-cell subpopulation is primed and expanded with MAGE-A4-derived peptides selected from Table 119 (Seq. ID. Nos. 1183-1192). In some embodiments, the donor cell source is HLA-DRB1*1101, and the MAGE-A4 targeted T-cell subpopulation is primed and expanded with MAGE-A4-derived peptides comprising the peptides of Table 119 (Seq. ID. Nos. 1183-1192). In some embodiments, the donor cell source is HLA-DRB1*1101, and the MAGE-A4 targeted T-cell subpopulation is primed and expanded with MAGE-A4-derived peptides comprising the peptides of Table 119 (Seq. ID. Nos. 1183-1192) and at least one additional set of peptides based on the donor cell source HLA-DR profile, wherein the at least one additional set of peptides are selected from the peptides of Tables 115-118 and 120. In some embodiments, the MAGE-A4-derived peptides also include one or more sets of HLA-A and HLA-B restricted peptides selected from Tables 101-114 (Seq. ID Nos. 1004-1142).

TABLE 119
MAGEA4 HLA-DRB1*1101 Epitope Peptides

| SEQ ID NO. | Sequence |
|---|---|
| 1183 | VKVLEHVVRVNARVR |
| 1184 | RYEFLWGPRALAETS |
| 1185 | PARYEFLWGPRALAE |
| 1186 | ELAHFLLRKYRAKEL |
| 1187 | KRCFPVIFGKASESL |
| 1188 | YLEYRQVPGSNPARY |
| 1189 | TSYVKVLEHVVRVNA |
| 1190 | SNKVDELAHFLLRKY |
| 1191 | KMIFGIDVKEVDPAS |
| 1192 | AEMLERVIKNYKRCF |

In some embodiments, the donor cell source is HLA-DRB1*1501, and the MAGE-A4 targeted T-cell subpopulation is primed and expanded with one or more MAGE-A4-derived peptides selected from Table 120 (Seq. ID. Nos. 1193-1202). In some embodiments, the donor cell source is HLA-DRB1*1501, and the MAGE-A4 targeted T-cell subpopulation is primed and expanded with MAGE-A4-derived peptides selected from Table 120 (Seq. ID. Nos. 1193-1202). In some embodiments, the donor cell source is HLA-DRB1*1501, and the MAGE-A4 targeted T-cell subpopulation is primed and expanded with MAGE-A4-derived peptides comprising the peptides of Table 120 (Seq. ID. Nos. 1193-1202). In some embodiments, the donor cell source is HLA-DRB1*1501, and the MAGE-A4 targeted T-cell subpopulation is primed and expanded with MAGE-A4-derived peptides comprising the peptides of Table 120 (Seq. ID. Nos. 1193-1202) and at least one additional set of peptides based on the donor cell source HLA-DR profile, wherein the at least one additional set of peptides are selected from the peptides of Tables 115-119. In some embodiments, the MAGE-A4-derived peptides also include one or more sets of HLA-A and HLA-B restricted peptides selected from Tables 101-114 (Seq. ID Nos. 1004-1142).

TABLE 120
MAGEA4 HLA-DRB1*1501 (DR2b) Epitope Peptides

| SEQ ID NO. | Sequence |
|---|---|
| 1193 | HFLLRKYRAKELVTK |
| 1194 | LGVMGVYDGREHTVY |
| 1195 | EAAVSSSSPLVPGTL |
| 1196 | ASALPTTISFTCWRQ |
| 1197 | ERVIKNYKRCFPVIF |
| 1198 | SESLKMIFGIDVKEV |
| 1199 | TCLGLSYDGLLGNNQ |
| 1200 | NNQIFPKTGLLIIVL |
| 1201 | GLLIIVLGTIAMEGD |
| 1202 | LIIVLGTIAMEGDSA |

SSX2 Antigenic Peptides

In some embodiments, the MUSTANG composition includes SSX2 (Synovial sarcoma, X breakpoint 2) specific T-cells. SSX2 specific T-cells can be generated as described below using one or more antigenic peptides to SSX2. In some embodiments, the SSX2 specific T-cells are generated using one or more antigenic peptides to SSX2, or a modified or heteroclitic peptide derived from a SSX2 peptide. In some embodiments, SSX2 specific T-cells are generated using a SSX2 antigen library comprising a pool of peptides (for example 15mers) containing amino acid overlap (for example 11 amino acids of overlap) between each sequence formed by scanning the protein amino acid sequence SEQ. ID. No. 1203 (UniProt KB-Q16385) for SSX2:

MNGDDAFARRPTVGAQIPEKIQKAFDDIAKYFSKEEWEKMKASEKIFYVY
MKRKYEAMTKLGFKATLPPFMCNKRAEDFQGNDLDNDPNRGNQVERPQMT
FGRLQGISPKIMPKKPAEEGNDSEEVPEASGPQNDGKELCPPGKPTTSEK
IHERSGPKRGEHAWTHRLRERKQLVIYEEISDPEEDDE.

Overlapping antigenic libraries are commercially available, for example, from JPT, for example, from JPT (Product Code: PM-SSX2 (Pep Mix™ Human (SSX2)). In some embodiments, the SSX2 specific T-cells are generated using a commercially available overlapping antigenic library made up of SSX2 peptides.

In some embodiments, the SSX2 specific T-cells are generated using one or more antigenic peptides to SSX2, or a modified or heteroclitic peptide derived from a SSX2 peptide. In some embodiments, the SSX2 specific T-cells are generated with peptides that recognize class I MHC molecules. In some embodiments, the SSX2 specific T-cells are generated with peptides that recognize class II MHC molecules. In some embodiments, the SSX2 specific T-cells are generated with peptides that recognize both class I and class II MHC molecules.

In some embodiments, the SSX2 peptides used to prime and expand a T-cell subpopulation includes specifically selected HLA-restricted peptides generated by determining the HLA profile of the donor source, and including peptides derived from SSX2 that best match the donor's HLA. In some embodiments, the SSX2 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting SSX2 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 121-127, the HLA-B peptides are selected from the peptides of Tables 128-134, and the HLA-DR peptides are selected from the peptides of Tables 135-140. For example, if the donor cell source has an HLA profile that is HLA-A*01/*02:01; HLA-B*15:01/*18; and HLA-DRB1*0101/*0301, then the SSX2 peptides used to prime and expand the SSX2 specific T-cell subpopulation are restricted to the specific HLA profile, and may include the peptides identified in Table 121 (Seq. ID. Nos. 1204-1213) for HLA-A*01; Table 122 (Seq. ID. Nos. 1214-1223) for HLA-A*02:01; Table 130 (Seq. ID. Nos. 1294-1303) for HLA-B*15:01; Table 131 (Seq. ID. Nos. 1304-1313) for HLA-B*18; Table 135 (Seq. ID. Nos. 1344-1353) for HLA-DRB1*0101; and Table 136 (Seq. ID. Nos. 1354-1363) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the SSX2 targeted T-cell subpopulation is primed and expanded with one or more SSX2-derived peptides selected from Table 121 (Seq. ID. Nos. 1204-1213). In some embodiments, the donor cell source is HLA-A*01, and the SSX2 targeted T-cell subpopulation is primed and expanded with SSX2-derived peptides selected from Table 121 (Seq. ID. Nos. 1204-1213). In some embodiments, the donor cell source is HLA-A*01, and the SSX2 targeted T-cell subpopulation is primed and expanded with SSX2-derived peptides comprising the peptides of Table 121 (Seq. ID. Nos. 1204-1213). In some embodiments, the donor cell source is HLA-A*01, and the SSX2 targeted T-cell subpopulation is primed and expanded with SSX2-derived peptides comprising the peptides of Table 121 (Seq. ID. Nos. 1204-1213) and at least one additional set of peptides based on the donor cell source HLA-A profile, wherein the at least one additional set of peptides are selected from the peptides of Tables 122-127. In some embodiments, the SSX2-derived peptides also include one or more sets of HLA-B and HLA-DR restricted peptides selected from Tables 128-140 (Seq. ID Nos. 1274-1403).

TABLE 121
SSX2 HLA-A*01 Epitope Peptides

| SEQ ID NO. | Sequence |
|---|---|
| 1204 | RLRERKQLVIY |
| 1205 | EKMKASEKIFY |
| 1206 | KIFYVYMKRKY |
| 1207 | IQKAFDDIAKY |
| 1208 | MKASEKIFYVY |
| 1209 | LRERKQLVIY |
| 1210 | IFYVYMKRKY |
| 1211 | ASEKIFYVY |
| 1212 | KAFDDIAKY |
| 1213 | FYVYMKRKY |

In some embodiments, the donor cell source is HLA-A*02:01, and the SSX2 targeted T-cell subpopulation is primed and expanded with one or more SSX2-derived peptides selected from Table 122 (Seq. ID. Nos. 1214-1223). In some embodiments, the donor cell source is HLA-A*02:01, and the SSX2 targeted T-cell subpopulation is primed and expanded with SSX2-derived peptides selected from Table 122 (Seq. ID. Nos. 1214-1223). In some embodiments, the donor cell source is HLA-A*02:01, and the SSX2 targeted T-cell subpopulation is primed and expanded with SSX2-derived peptides comprising the peptides of Table 122 (Seq. ID. Nos. 1214-1223). In some embodiments, the donor cell source is HLA-A*02:01, and the SSX2 targeted T-cell subpopulation is primed and expanded with SSX2-derived peptides comprising the peptides of Table 122 (Seq. ID. Nos. 1214-1223) and at least one additional set of peptides based on the donor cell source HLA-A profile, wherein the at least one additional set of peptides are selected from the peptides of Tables 121, and 123-127. In some embodiments, the SSX2-derived peptides also include one or more sets of HLA-B and HLA-DR restricted peptides selected from Tables 128-140 (Seq. ID Nos. 1274-1403).

TABLE 122
SSX2 HLA-A*02:01 Epitope Peptides

| SEQ ID NO. | Sequence |
|---|---|
| 1214 | RLRERKQLVI |
| 1215 | QMTFGRLQGI |
| 1216 | RLQGISPKI |
| 1217 | KASEKIFYV |
| 1218 | RLRERKQLV |
| 1219 | QIPEKIQKA |
| 1220 | MTFGRLQGI |
| 1221 | TKLGFKATL |
| 1222 | DAFARRPTV |
| 1223 | KIQKAFDDI |

In some embodiments, the donor cell source is HLA-A*03, and the SSX2 targeted T-cell subpopulation is primed and expanded with one or more SSX2-derived peptides selected from Table 123 (Seq. ID. Nos. 1224-1233). In some embodiments, the donor cell source is HLA-A*03, and the SSX2 targeted T-cell subpopulation is primed and expanded with SSX2-derived peptides selected from Table 123 (Seq. ID. Nos. 1224-1233). In some embodiments, the donor cell source is HLA-A*03, and the SSX2 targeted T-cell subpopulation is primed and expanded with SSX2-derived peptides comprising the peptides of Table 123 (Seq. ID. Nos. 1224-1233). In some embodiments, the donor cell source is HLA-A*03, and the SSX2 targeted T-cell subpopulation is primed and expanded with SSX2-derived peptides comprising the peptides of Table 123 (Seq. ID. Nos. 1224-1233) and at least one additional set of peptides based on the donor cell source HLA-A profile, wherein the at least one additional set of peptides are selected from the peptides of Tables 121-122 and 124-127. In some embodiments, the SSX2-derived peptides also include one or more sets of HLA-B and HLA-DR restricted peptides selected from Tables 128-140 (Seq. ID Nos. 1274-1403).

TABLE 123
SSX2 HLA-A*03 Epitope Peptides

| SEQ ID NO. | Sequence |
|---|---|
| 1224 | RLRERKQLVI |
| 1225 | KIFYVYMKRK |
| 1226 | KIHERSGPK |
| 1227 | QVERPQMTF |
| 1228 | TLPPFMCNK |
| 1229 | GISPKIMPK |
| 1230 | TVGAQIPEK |
| 1231 | AQIPEKIQK |
| 1232 | KRKYEAMTK |
| 1233 | ARRPTVGAQI |

In some embodiments, the donor cell source is HLA-A*11:01, and the SSX2 targeted T-cell subpopulation is primed and expanded with one or more SSX2-derived peptides selected from Table 124 (Seq. ID. Nos. 1234-1243). In some embodiments, the donor cell source is HLA-A*11:01, and the SSX2 targeted T-cell subpopulation is primed and expanded with SSX2-derived peptides selected from Table 124 (Seq. ID. Nos. 1234-1243). In some embodiments, the donor cell source is HLA-A*11:01, and the SSX2 targeted T-cell subpopulation is primed and expanded with SSX2-derived peptides comprising the peptides of Table 124 (Seq. ID. Nos. 1234-1243). In some embodiments, the donor cell source is HLA-A*11:01, and the SSX2 targeted T-cell subpopulation is primed and expanded with SSX2-derived peptides comprising the peptides of Table 124 (Seq. ID. Nos. 1234-1243), and at least one additional set of peptides based on the donor cell source HLA-A profile, wherein the at least one additional set of peptides are selected from the peptides of Tables 121-123 and 125-127. In some embodiments, the SSX2-derived peptides also include one or more sets of HLA-B and HLA-DR restricted peptides selected from Tables 128-140 (Seq. ID Nos. 1274-1403).

TABLE 124
SSX2 HLA-A*11:01 Epitope Peptides

| SEQ ID NO. | Sequence |
|---|---|
| 1234 | ATLPPFMCNK |
| 1235 | PTVGAQIPEK |
| 1236 | PTTSEKIHER |
| 1237 | FSKEEWEKMK |
| 1238 | ASGPQNDGK |
| 1239 | TVGAQIPEK |
| 1240 | GISPKIMPK |
| 1241 | TTSEKIHER |
| 1242 | WTHRLRERK |
| 1243 | AQIPEKIQK |

In some embodiments, the donor cell source is HLA-A*24:02, and the SSX2 targeted T-cell subpopulation is primed and expanded with one or more SSX2-derived peptides selected from Table 125 (Seq. ID. Nos. 1244-1253). In some embodiments, the donor cell source is HLA-A*24:02, and the SSX2 targeted T-cell subpopulation is primed and expanded with SSX2-derived peptides selected from Table 125 (Seq. ID. Nos. 1244-1253). In some embodiments, the donor cell source is HLA-A*24:02, and the SSX2 targeted T-cell subpopulation is primed and expanded with SSX2-derived peptides comprising the peptides of Table 125 (Seq. ID. Nos. 1244-1253). In some embodiments, the donor cell source is HLA-A*24:02, and the SSX2 targeted T-cell subpopulation is primed and expanded with SSX2-derived peptides comprising the peptides of Table 125 (Seq. ID. Nos. 1244-1253), and at least one additional set of peptides based on the donor cell source HLA-A profile, wherein the at least one additional set of peptides are selected from the peptides of Tables 121-124 and 126-127. In some embodiments, the SSX2-derived peptides also include one or more sets of HLA-B and HLA-DR restricted peptides selected from Tables 128-140 (Seq. ID Nos. 1274-1403).

TABLE 125
SSX2 HLA-A*24:02 Epitope Peptides

| SEQ ID NO. | Sequence |
|---|---|
| 1244 | KYEAMTKLGF |
| 1245 | TVGAQIPEKI |
| 1246 | QIPEKIQKAF |
| 1247 | VYMKRKYEAM |
| 1248 | GRLQGISPKI |
| 1249 | AFDDIAKYF |
| 1250 | VGAQIPEKI |
| 1251 | GFKATLPPF |
| 1252 | GKPTTSEKI |
| 1253 | KQLVIYEEI |

In some embodiments, the donor cell source is HLA-A*26, and the SSX2 targeted T-cell subpopulation is primed and expanded with one or more SSX2-derived peptides selected from Table 126 (Seq. ID. Nos. 1254-1263). In some embodiments, the donor cell source is HLA-A*26, and the SSX2 targeted T-cell subpopulation is primed and expanded with SSX2-derived peptides selected from Table 126 (Seq. ID. Nos. 1254-1263). In some embodiments, the donor cell source is HLA-A*26, and the SSX2 targeted T-cell subpopulation is primed and expanded with SSX2-derived peptides comprising the peptides of Table 126 (Seq. ID. Nos. 1254-1263). In some embodiments, the donor cell source is HLA-A*26, and the SSX2 targeted T-cell subpopulation is primed and expanded with SSX2-derived peptides comprising the peptides of Table 126 (Seq. ID. Nos. 1254-1263) and at least one additional set of peptides based on the donor cell source HLA-A profile, wherein the at least one additional set of peptides are selected from the peptides of Tables 121-125 and 127. In some embodiments, the SSX2-derived peptides also include one or more sets of HLA-B and HLA-DR restricted peptides selected from Tables 128-140 (Seq. ID Nos. 1274-1403).

TABLE 126
SSX2 HLA-A*26 Epitope Peptides

| SEQ ID NO. | Sequence |
|---|---|
| 1254 | EVPEASGPQN |
| 1255 | ERPQMTFGRL |
| 1256 | LVIYEEISDP |
| 1257 | EKMKASEKIF |
| 1258 | MTKLGFKATL |
| 1259 | KAFDDIAKY |
| 1260 | EKIFYVYMK |
| 1261 | QVERPQMTF |
| 1262 | EAMTKLGFK |
| 1263 | MTFGRLQGI |

In some embodiments, the donor cell source is HLA-A*68:01, and the SSX2 targeted T-cell subpopulation is primed and expanded with one or more SSX2-derived peptides selected from Table 127 (Seq. ID. Nos. 1264-1273). In some embodiments, the donor cell source is HLA-A*68:01, and the SSX2 targeted T-cell subpopulation is primed and expanded with SSX2-derived peptides selected from Table 127 (Seq. ID. Nos. 1264-1273). In some embodiments, the donor cell source is HLA-A*68:01, and the SSX2 targeted T-cell subpopulation is primed and expanded with SSX2-derived peptides comprising the peptides of Table 127 (Seq. ID. Nos. 1264-1273). In some embodiments, the donor cell source is HLA-A*68:01, and the SSX2 targeted T-cell subpopulation is primed and expanded with SSX2-derived peptides comprising the peptides of Table 127 (Seq. ID. Nos. 1264-1273), and at least one additional set of peptides based on the donor cell source HLA-A profile, wherein the at least one additional set of peptides are selected from the peptides of Tables 121-126. In some embodiments, the SSX2-derived peptides also include one or more sets of HLA-B and HLA-DR restricted peptides selected from Tables 128-140 (Seq. ID Nos. 1274-1403).

TABLE 127
SSX2 HLA-A*68:01 Epitope Peptides

| SEQ ID NO. | Sequence |
|---|---|
| 1264 | ATLPPFMCNKR |
| 1265 | KIQKAFDDIAK |
| 1266 | QVERPQMTFGR |
| 1267 | GAQIPEKIQK |
| 1268 | DPNRGNQVER |
| 1269 | EASGPQNDGK |
| 1270 | TTSEKIHER |
| 1271 | EAMTKLGFK |
| 1272 | TVGAQIPEK |
| 1273 | GISPKIMPK |

In some embodiments, the donor cell source is HLA-B*07:02, and the SSX2 targeted T-cell subpopulation is primed and expanded with one or more SSX2-derived peptides selected from Table 128 (Seq. ID. Nos. 1274-1283). In some embodiments, the donor cell source is HLA-B*07:02, and the SSX2 targeted T-cell subpopulation is primed and expanded with SSX2-derived peptides selected from Table 128 (Seq. ID. Nos. 1274-1283). In some embodiments, the donor cell source is HLA-B*07:02, and the SSX2 targeted T-cell subpopulation is primed and expanded with SSX2-derived peptides comprising the peptides of Table 128 (Seq. ID. Nos. 1274-1283). In some embodiments, the donor cell source is HLA-B*07:02, and the SSX2 targeted T-cell subpopulation is primed and expanded with SSX2-derived peptides comprising the peptides of Table 128 (Seq. ID. Nos. 1274-1283), and at least one additional set of peptides based on the donor cell source HLA-B profile, wherein the at least one additional set of peptides are selected from the peptides of Tables 129-134. In some embodiments, the SSX2-derived peptides also include one or more sets of HLA-A and HLA-DR restricted peptides selected from Tables 121-127 and 135-140 (Seq. ID Nos. 1204-1273 and 1344-1403).

TABLE 128
SSX2 HLA-B*07:02 Epitope Peptides

| SEQ ID NO. | Sequence |
|---|---|
| 1274 | SPKIMPKKPA |
| 1275 | GPKRGEHAWT |
| 1276 | LPPFMCNKRA |
| 1277 | RPQMTFGRL |
| 1278 | GPQNDGKEL |
| 1279 | IPEKIQKAF |
| 1280 | PPGKPTTSE |
| 1281 | FARRPTVGA |
| 1282 | AEDFQGNDL |
| 1283 | MPKKPAEEG |

In some embodiments, the donor cell source is HLA-B*08, and the SSX2 targeted T-cell subpopulation is primed and expanded with one or more SSX2-derived peptides selected from Table 129 (Seq. ID. Nos. 1284-1293). In some embodiments, the donor cell source is HLA-B*08, and the SSX2 targeted T-cell subpopulation is primed and expanded with SSX2-derived peptides selected from Table 129 (Seq. ID. Nos. 1284-1293). In some embodiments, the donor cell source is HLA-B*08, and the SSX2 targeted T-cell subpopulation is primed and expanded with SSX2-derived peptides comprising the peptides of Table 129 (Seq. ID. Nos. 1284-1293). In some embodiments, the donor cell source is HLA-B*08, and the SSX2 targeted T-cell subpopulation is primed and expanded with SSX2-derived peptides comprising the peptides of Table 129 (Seq. ID. Nos. 1284-1293) and at least one additional set of peptides based on the donor cell source HLA-B profile, wherein the at least one additional set of peptides are selected from the peptides of Tables 128 and 130-134. In some embodiments, the SSX2-derived peptides also include one or more sets of HLA-A and HLA-DR restricted peptides selected from Tables 121-127 and 135-140 (Seq. ID Nos. 1204-1273 and 1344-1403).

TABLE 129
SSX2 HLA-B*08 Epitope Peptides

| SEQ ID NO. | Sequence |
|---|---|
| 1284 | WEKMKASEK |
| 1285 | YMKRKYEAM |
| 1286 | SPKIMPKKP |
| 1287 | GPQNDGKEL |
| 1288 | RLRERKQL |
| 1289 | KLGFKATL |
| 1290 | EAMTKLGF |
| 1291 | IQKAFDDI |
| 1292 | GPKRGEHA |
| 1293 | GFKATLPPF |

In some embodiments, the donor cell source is HLA-B*15:01, and the SSX2 targeted T-cell subpopulation is primed and expanded with one or more SSX2-derived peptides selected from Table 130 (Seq. ID. Nos. 1294-1303). In some embodiments, the donor cell source is HLA-B*15:01, and the SSX2 targeted T-cell subpopulation is primed and expanded with SSX2-derived peptides selected from Table 130 (Seq. ID. Nos. 1294-1303). In some embodiments, the donor cell source is HLA-B*15:01, and the SSX2 targeted T-cell subpopulation is primed and expanded with SSX2-derived peptides comprising the peptides of Table 130 (Seq. ID. Nos. 1294-1303). In some embodiments, the donor cell source is HLA-B*15:01, and the SSX2 targeted T-cell subpopulation is primed and expanded with SSX2-derived peptides comprising the peptides of Table 130 (Seq. ID. Nos. 1294-1303) and at least one additional set of peptides based on the donor cell source HLA-B profile, wherein the at least one additional set of peptides are selected from the peptides of Tables 128-129 and 131-134. In some embodiments, the SSX2-derived peptides also include one or more sets of HLA-A and HLA-DR restricted peptides selected from Tables 121-127 and 135-140 (Seq. ID Nos. 1204-1273 and 1344-1403).

TABLE 130
SSX2 HLA-B*15:01 (B62) Epitope Peptides

| SEQ ID NO. | Sequence |
|---|---|
| 1294 | NQVERPQMTF |
| 1295 | AQIPEKIQKA |
| 1296 | KASEKIFYVY |
| 1297 | RLRERKQLVI |
| 1298 | RLQGISPKIM |
| 1299 | ELCPPGKPTT |
| 1300 | KAFDDIAKY |
| 1301 | AQIPEKIQK |
| 1302 | PQNDGKELC |
| 1303 | RLRERKQLV |

In some embodiments, the donor cell source is HLA-B*18, and the SSX2 targeted T-cell subpopulation is primed and expanded with one or more SSX2-derived peptides selected from Table 131 (Seq. ID. Nos. 1304-1313). In some embodiments, the donor cell source is HLA-B*18, and the SSX2 targeted T-cell subpopulation is primed and expanded with SSX2-derived peptides selected from Table 131 (Seq. ID. Nos. 1304-1313). In some embodiments, the donor cell source is HLA-B*18, and the SSX2 targeted T-cell subpopulation is primed and expanded with SSX2-derived peptides comprising the peptides of Table 131 (Seq. ID. Nos. 1304-1313). In some embodiments, the donor cell source is HLA-B*18, and the SSX2 targeted T-cell subpopulation is primed and expanded with SSX2-derived peptides comprising the peptides of Table 131 (Seq. ID. Nos. 1304-1313) and at least one additional set of peptides based on the donor cell source HLA-B profile, wherein the at least one additional set of peptides are selected from the peptides of Tables 128-130 and 132-134. In some embodiments, the SSX2-derived peptides also include one or more sets of HLA-A and HLA-DR restricted peptides selected from Tables 121-127 and 135-140 (Seq. ID Nos. 1204-1273 and 1344-1403).

TABLE 131
SSX2 HLA-B*18 Epitope Peptides

| SEQ ID NO. | Sequence |
|---|---|
| 1304 | RERKQLVIY |
| 1305 | YEAMTKLGF |
| 1306 | AEDFQGNDL |
| 1307 | GEHAWTHRL |
| 1308 | EEISDPEED |
| 1309 | SEKIFYVY |
| 1310 | PEKIQKAF |
| 1311 | VERPQMTF |
| 1312 | EEWEKMKA |
| 1313 | EEVPEASG |

In some embodiments, the donor cell source is HLA-B*27:05, and the SSX2 targeted T-cell subpopulation is primed and expanded with one or more SSX2-derived peptides selected from Table 132 (Seq. ID. Nos. 1314-1323). In some embodiments, the donor cell source is HLA-B*27:05, and the SSX2 targeted T-cell subpopulation is primed and expanded with SSX2-derived peptides selected from Table 132 (Seq. ID. Nos. 1314-1323). In some embodiments, the donor cell source is HLA-B*27:05, and the SSX2 targeted T-cell subpopulation is primed and expanded with SSX2-derived peptides comprising the peptides of Table 132 (Seq. ID. Nos. 1314-1323). In some embodiments, the donor cell source is HLA-B*27:05, and the SSX2 targeted T-cell subpopulation is primed and expanded with SSX2-derived peptides comprising the peptides of Table 132 (Seq. ID. Nos. 1314-1323) and at least one additional set of peptides based on the donor cell source HLA-B profile, wherein the at least one additional set of peptides are selected from the peptides of Tables 128-131 and 133-134. In some embodiments, the SSX2-derived peptides also include one or more sets of HLA-A and HLA-DR restricted peptides selected from Tables 121-127 and 135-140 (Seq. ID Nos. 1204-1273 and 1344-1403).

TABLE 132
SSX2 HLA-B*27:05 Epitope Peptides

| SEQ ID NO. | Sequence |
|---|---|
| 1314 | GRLQGISPKI |
| 1315 | KRKYEAMTKL |
| 1316 | KRGEHAWTHR |
| 1317 | ERPQMTFGRL |
| 1318 | ERSGPKRGEH |
| 1319 | GRLQGISPK |
| 1320 | KRKYEAMTK |
| 1321 | HRLRERKQL |
| 1322 | RRPTVGAQI |
| 1323 | LRERKQLVI |

In some embodiments, the donor cell source is HLA-B*35:01, and the SSX2 targeted T-cell subpopulation is primed and expanded with one or more SSX2-derived peptides selected from Table 133 (Seq. ID. Nos. 1324-1333). In some embodiments, the donor cell source is HLA-B*35:01, and the SSX2 targeted T-cell subpopulation is primed and expanded with SSX2-derived peptides selected from Table 133 (Seq. ID. Nos. 1324-1333). In some embodiments, the donor cell source is HLA-B*35:01, and the SSX2 targeted T-cell subpopulation is primed and expanded with SSX2-derived peptides comprising the peptides of Table 133 (Seq. ID. Nos. 1324-1333). In some embodiments, the donor cell source is HLA-B*35:01, and the SSX2 targeted T-cell subpopulation is primed and expanded with SSX2-derived peptides comprising the peptides of Table 133 (Seq. ID. Nos. 1324-1333) and at least one additional set of peptides based on the donor cell source HLA-B profile, wherein the at least one additional set of peptides are selected from the peptides of Tables 128-132 and 134. In some embodiments, the SSX2-derived peptides also include one or more sets of HLA-A and HLA-DR restricted peptides selected from Tables 121-127 and 135-140 (Seq. ID Nos. 1204-1273 and 1344-1403).

TABLE 133
SSX2 HLA-B*35:01 Epitope Peptides

| SEQ ID NO. | Sequence |
|---|---|
| 1324 | THRLRERKQL |
| 1325 | KRKYEAMTKL |
| 1326 | ERPQMTFGRL |
| 1327 | GRLQGISPKI |
| 1328 | IHERSGPKRG |
| 1329 | LRERKQLVI |
| 1330 | RRPTVGAQI |
| 1331 | AFDDIAKYF |
| 1332 | TKLGFKATL |
| 1333 | AEDFQGNDL |

In some embodiments, the donor cell source is HLA-B*58:02, and the SSX2 targeted T-cell subpopulation is primed and expanded with one or more SSX2-derived peptides selected from Table 134 (Seq. ID. Nos. 1334-1343). In some embodiments, the donor cell source is HLA-B*58:02, and the SSX2 targeted T-cell subpopulation is primed and expanded with SSX2-derived peptides selected from Table 134 (Seq. ID. Nos. 1334-1343). In some embodiments, the donor cell source is HLA-B*58:02, and the SSX2 targeted T-cell subpopulation is primed and expanded with SSX2-derived peptides comprising the peptides of Table 134 (Seq. ID. Nos. 1334-1343). In some embodiments, the donor cell source is HLA-B*58:02, and the SSX2 targeted T-cell subpopulation is primed and expanded with SSX2-derived peptides comprising the peptides of Table 134 (Seq. ID. Nos. 1334-1343) and at least one additional set of peptides based on the donor cell source HLA-B profile, wherein the at least one additional set of peptides are selected from the peptides of Tables 128-133. In some embodiments, the SSX2-derived peptides also include one or more sets of HLA-A and HLA-DR restricted peptides selected from Tables 121-127 and 135-140 (Seq. ID Nos. 1204-1273 and 1344-1403).

TABLE 134
SSX2 HLA-B*58:02 Epitope Peptides

| SEQ ID NO. | Sequence |
|---|---|
| 1334 | KAFDDIAKYF |
| 1335 | KASEKIFYVY |
| 1336 | RAEDFQGNDL |
| 1337 | MTKLGFKATL |
| 1338 | ASEKIFYVYM |
| 1339 | IAKYFSKEEW |
| 1340 | KAFDDIAKY |
| 1341 | FSKEEWEKM |
| 1342 | ASEKIFYVY |
| 1343 | QVERPQMTF |

In some embodiments, the donor cell source is HLA-DRB1*0101, and the SSX2 targeted T-cell subpopulation is primed and expanded with one or more SSX2-derived peptides selected from Table 135 (Seq. ID. Nos. 1344-1353). In some embodiments, the donor cell source is HLA-DRB1*0101, and the SSX2 targeted T-cell subpopulation is primed and expanded with SSX2-derived peptides selected from Table 135 (Seq. ID. Nos. 1344-1353). In some embodiments, the donor cell source is HLA-DRB1*0101, and the SSX2 targeted T-cell subpopulation is primed and expanded with SSX2-derived peptides comprising the peptides of Table 135 (Seq. ID. Nos. 1344-1353). In some embodiments, the donor cell source is HLA-DRB1*0101, and the SSX2 targeted T-cell subpopulation is primed and expanded with SSX2-derived peptides comprising the peptides of Table 135 (Seq. ID. Nos. 1344-1353) and at least one additional set of peptides based on the donor cell source HLA-DR profile, wherein the at least one additional set of peptides are selected from the peptides of Tables 136-140. In some embodiments, the SSX2-derived peptides also include one or more sets of HLA-A and HLA-B restricted peptides selected from Tables 121-134 (Seq. ID Nos. 1204-1343).

TABLE 135
SSX2 HLA-DRB1*0101 Epitope Peptides

| SEQ ID NO. | Sequence |
|---|---|
| 1344 | QMTFGRLQGISPKIM |
| 1345 | RPTVGAQIPEKIQKA |
| 1346 | FGRLQGISPKIMPKK |
| 1347 | DDAFARRPTVGAQIP |
| 1348 | KEEWEKMKASEKIFY |
| 1349 | KRKYEAMTKLGFKAT |
| 1350 | KLGFKATLPPFMCNK |
| 1351 | QKAFDDIAKYFSKEE |
| 1352 | PPFMCNKRAEDFQGN |
| 1353 | QGISPKIMPKKPAEE |

In some embodiments, the donor cell source is HLA-DRB1*0301, and the SSX2 targeted T-cell subpopulation is primed and expanded with one or more SSX2-derived peptides selected from Table 136 (Seq. ID. Nos. 1354-1363). In some embodiments, the donor cell source is HLA-DRB1*0301, and the SSX2 targeted T-cell subpopulation is primed and expanded with SSX2-derived peptides selected from Table 136 (Seq. ID. Nos. 1354-1363). In some embodiments, the donor cell source is HLA-DRB1*0301, and the SSX2 targeted T-cell subpopulation is primed and expanded with SSX2-derived peptides comprising the peptides of Table 136 (Seq. ID. Nos. 1354-1363). In some embodiments, the donor cell source is HLA-DRB1*0301, and the SSX2 targeted T-cell subpopulation is primed and expanded with SSX2-derived peptides comprising the peptides of Table 136 (Seq. ID. Nos. 1354-1363) and at least one additional set of peptides based on the donor cell source HLA-DR profile, wherein the at least one additional set of peptides are selected from the peptides of Tables 135 and 137-140. In some embodiments, the SSX2-derived peptides also include one or more sets of HLA-A and HLA-B restricted peptides selected from Tables 121-134 (Seq. ID Nos. 1204-1343).

TABLE 136
SSX2 HLA-DRB1*0301 (DR17) Epitope Peptides

| SEQ ID NO. | Sequence |
|---|---|
| 1354 | GNDLDNDPNRGNQVE |
| 1355 | GAQIPEKIQKAFDDI |
| 1356 | WEKMKASEKIFYVYM |
| 1357 | EKIFYVYMKRKYEAM |
| 1358 | KIQKAFDDIAKYFSK |
| 1359 | THRLRERKQLVIYEE |
| 1360 | IQKAFDDIAKYFSKE |
| 1361 | YVYMKRKYEAMTKLG |
| 1362 | PPFMCNKRAEDFQGN |
| 1363 | CNKRAEDFQGNDLDN |

In some embodiments, the donor cell source is HLA-DRB1*0401, and the SSX2 targeted T-cell subpopulation is primed and expanded with one or more SSX2-derived peptides selected from Table 137 (Seq. ID. Nos. 1364-1373). In some embodiments, the donor cell source is HLA-DRB1*0401, and the SSX2 targeted T-cell subpopulation is primed and expanded with SSX2-derived peptides selected from Table 137 (Seq. ID. Nos. 1364-1373). In some embodiments, the donor cell source is HLA-DRB1*0401, and the SSX2 targeted T-cell subpopulation is primed and expanded with SSX2-derived peptides comprising the peptides of Table 137 (Seq. ID. Nos. 1364-1373). In some embodiments, the donor cell source is HLA-DRB1*0401, and the SSX2 targeted T-cell subpopulation is primed and expanded with SSX2-derived peptides comprising the peptides of Table 137 (Seq. ID. Nos. 1364-1373) and at least one additional set of peptides based on the donor cell source HLA-DR profile, wherein the at least one additional set of peptides are selected from the peptides of Tables 135-136 and 138-140. In some embodiments, the SSX2-derived peptides also include one or more sets of HLA-A and HLA-B restricted peptides selected from Tables 121-134 (Seq. ID Nos. 1204-1343).

TABLE 137
SSX2 HLA-DRB1*0401 (DR4Dw4) Epitope Peptides

| SEQ ID NO. | Sequence |
|---|---|
| 1364 | GNDLDNDPNRGNQVE |
| 1365 | QKAFDDIAKYFSKEE |
| 1366 | AKYFSKEEWEKMKAS |
| 1367 | PEKIQKAFDDIAKYF |
| 1368 | SEKIFYVYMKRKYEA |
| 1369 | YEAMTKLGFKATLPP |
| 1370 | FGRLQGISPKIMPKK |
| 1371 | SEEVPEASGPQNDGK |
| 1372 | QLVIYEEISDPEEDD |
| 1373 | MNGDDAFARRPTVGA |

In some embodiments, the donor cell source is HLA-DRB1*0701, and the SSX2 targeted T-cell subpopulation is primed and expanded with one or more SSX2-derived peptides selected from Table 138 (Seq. ID. Nos. 1374-1383). In some embodiments, the donor cell source is HLA-DRB1*0701, and the SSX2 targeted T-cell subpopulation is primed and expanded with SSX2-derived peptides selected from Table 138 (Seq. ID. Nos. 1374-1383). In some embodiments, the donor cell source is HLA-DRB1*0701, and the SSX2 targeted T-cell subpopulation is primed and expanded with SSX2-derived peptides comprising the peptides of Table 138 (Seq. ID. Nos. 1374-1383). In some embodiments, the donor cell source is HLA-DRB1*0701, and the SSX2 targeted T-cell subpopulation is primed and expanded with SSX2-derived peptides comprising the peptides of Table 138 (Seq. ID. Nos. 1374-1383) and at least one additional set of peptides based on the donor cell source HLA-DR profile, wherein the at least one additional set of peptides are selected from the peptides of Tables 135-137 and 139-140. In some embodiments, the SSX2-derived peptides also include one or more sets of HLA-A and HLA-B restricted peptides selected from Tables 121-134 (Seq. ID Nos. 1204-1343).

TABLE 138
SSX2 HLA-DRB1*0701 Epitope Peptides

| SEQ ID NO. | Sequence |
|---|---|
| 1374 | KLGFKATLPPFMCNK |
| 1375 | PEKIQKAFDDIAKYF |
| 1376 | CPPGKPTTSEKIHER |
| 1377 | DDAFARRPTVGAQIP |
| 1378 | FGRLQGISPKIMPKK |
| 1379 | PKRGEHAWTHRLRER |
| 1380 | LVIYEEISDPEEDDE |
| 1381 | WEKMKASEKIFYVYM |
| 1382 | AKYFSKEEWEKMKAS |
| 1383 | EWEKMKASEKIFYVY |

In some embodiments, the donor cell source is HLA-DRB1*1101, and the SSX2 targeted T-cell subpopulation is primed and expanded with one or more SSX2-derived peptides selected from Table 139 (Seq. ID. Nos. 1384-1393). In some embodiments, the donor cell source is HLA-DRB1*1101, and the SSX2 targeted T-cell subpopulation is primed and expanded with SSX2-derived peptides selected from Table 139 (Seq. ID. Nos. 1384-1393). In some embodiments, the donor cell source is HLA-DRB1*1101, and the SSX2 targeted T-cell subpopulation is primed and expanded with SSX2-derived peptides comprising the peptides of Table 139 (Seq. ID. Nos. 1384-1393). In some embodiments, the donor cell source is HLA-DRB1*1101, and the SSX2 targeted T-cell subpopulation is primed and expanded with SSX2-derived peptides comprising the peptides of Table 139 (Seq. ID. Nos. 1384-1393) and at least one additional set of peptides based on the donor cell source HLA-DR profile, wherein the at least one additional set of peptides are selected from the peptides of Tables 135-138 and 140. In some embodiments, the SSX2-derived peptides also include one or more sets of HLA-A and HLA-B restricted peptides selected from Tables 121-134 (Seq. ID Nos. 1204-1343).

TABLE 139
SSX2 HLA-DRB1*1101 Epitope Peptides

| SEQ ID NO. | Sequence |
|---|---|
| 1384 | QKAFDDIAKYFSKEE |
| 1385 | KIFYVYMKRKYEAMT |
| 1386 | KRKYEAMTKLGFKAT |
| 1387 | QMTFGRLQGISPKIM |
| 1388 | EHAWTHRLRERKQLV |
| 1389 | EKIFYVYMKRKYEAM |
| 1390 | IFYVYMKRKYEAMTK |
| 1391 | GISPKIMPKKPAEEG |
| 1392 | LPPFMCNKRAEDFQG |
| 1393 | DDAFARRPTVGAQIP |

In some embodiments, the donor cell source is HLA-DRB1*1501, and the SSX2 targeted T-cell subpopulation is primed and expanded with one or more SSX2-derived peptides selected from Table 140 (Seq. ID. Nos. 1394-1403). In some embodiments, the donor cell source is HLA-DRB1*1501, and the SSX2 targeted T-cell subpopulation is primed and expanded with SSX2-derived peptides selected from Table 140 (Seq. ID. Nos. 1394-1403). In some embodiments, the donor cell source is HLA-DRB1*1501, and the SSX2 targeted T-cell subpopulation is primed and expanded with SSX2-derived peptides comprising the peptides of Table 140 (Seq. ID. Nos. 1394-1403). In some embodiments, the donor cell source is HLA-DRB1*1501, and the SSX2 targeted T-cell subpopulation is primed and expanded with SSX2-derived peptides comprising the peptides of Table 140 (Seq. ID. Nos. 1394-1403) and at least one additional set of peptides based on the donor cell source HLA-DR profile, wherein the at least one additional set of peptides are selected from the peptides of Tables 135-139. In some embodiments, the SSX2-derived peptides also include one or more sets of HLA-A and HLA-B restricted peptides selected from Tables 121-134 (Seq. ID Nos. 1204-1343).

TABLE 140
SSX2 HLA-DRB1*1501 (DR2b) Epitope Peptides

| SEQ ID NO. | Sequence |
|---|---|
| 1394 | RKQLVIYEEISDPEE |
| 1395 | FDDIAKYFSKEEWEK |
| 1396 | VYMKRKYEAMTKLGF |
| 1397 | MTKLGFKATLPPFMC |
| 1398 | KATLPPFMCNKRAED |
| 1399 | DDAFARRPTVGAQIP |
| 1400 | QMTFGRLQGISPKIM |
| 1401 | EKIQKAFDDIAKYFS |
| 1402 | YFSKEEWEKMKASEK |
| 1403 | ASEKIFYVYMKRKYE |

PR3 Antigenic Peptides

In some embodiments, the MUSTANG composition includes PR3 (leukocyte proteinase 3) specific T-cells. PR3 specific T-cells can be generated as described below using one or more antigenic peptides to PR3. In some embodiments, the PR3 specific T-cells are generated using one or more antigenic peptides to PR3, or a modified or heteroclitic peptide derived from a PR3 peptide. In some embodiments, PR3 specific T-cells are generated using a PR3 antigen library comprising a pool of peptides (for example 15mers) containing amino acid overlap (for example 11 amino acids of overlap) between each sequence formed by scanning the protein amino acid sequence SEQ. ID. No. 1404 (UniProt KB-P24158) for PR3:

MAHRPPSPALASVLLALLLSGAARAAEIVGGHEAQPHSRPYMASLQMRGNP
GSHFCGGTLIHPSFVLTAAHCLRDIPQRLVNVVLGAHNVRTQEPTQQHFSV
AQVFLNNYDAENKLNDVLLIQLSSPANLSASVATVQLPQQDQPVPHGTQCL
AMGWGRVGAHDPPAQVLQELNVTVVTFFCRPHNICTFVPRRKAGICFGDSG
GPLICDGIIQGIDSFVIWGCATRLFPDFFTRVALYVDWIRSTLRRVEAKGR
P.

In some embodiments, the PR3 specific T-cells are generated using one or more antigenic peptides to PR3, or a modified or heteroclitic peptide derived from a PR3 peptide. In some embodiments, the PR3 specific T-cells are generated with peptides that recognize class I MHC molecules. In some embodiments, the PR3 specific T-cells are generated with peptides that recognize class II MHC molecules. In some embodiments, the PR3 specific T-cells are generated with peptides that recognize both class I and class II MHC molecules.

In some embodiments, the PR3 peptides used to prime and expand a T-cell subpopulation includes specifically selected HLA-restricted peptides generated by determining the HLA profile of the donor source, and including peptides derived from PR3 that best match the donor's HLA. In some embodiments, the PR3 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting PR3 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 141-147, the HLA-B peptides are selected from the peptides of Tables 148-154, and the HLA-DR peptides are selected from the peptides of Tables 155-160. For example, if the donor cell source has an HLA profile that is HLA-A*01/*02:01; HLA-B*15:01/*18; and HLA-DRB1*0101/*0301, then the PR3 peptides used to prime and expand the PR3 specific T-cell subpopulation are restricted to the specific HLA profile, and may include the peptides identified in Table 141 (Seq. ID. Nos. 1405-1414) for HLA-A*01; Table 142 (Seq. ID. Nos. 1415-1424) for HLA-A*02:01; Table 150 (Seq. ID. Nos. 1495-1504) for HLA-B*15:01; Table 151 (Seq. ID. Nos. 1505-1514) for HLA-B*18; Table 155 (Seq. ID. Nos. 1545-1554) for HLA-DRB1*0101; and Table 156 (Seq. ID. Nos. 1555-1564) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the PR3 targeted T-cell subpopulation is primed and expanded with one or more PR3-derived peptides selected from Table 141 (Seq. ID. Nos. 1405-1414). In some embodiments, the donor cell source is HLA-A*01, and the PR3 targeted T-cell subpopulation is primed and expanded with PR3-derived peptides selected from Table 141 (Seq. ID. Nos. 1405-1414). In some embodiments, the donor cell source is HLA-A*01, and the PR3 targeted T-cell subpopulation is primed and expanded with PR3-derived peptides comprising the peptides of Table 141 (Seq. ID. Nos. 1405-1414). In some embodiments, the donor cell source is HLA-A*01, and the PR3 targeted T-cell subpopulation is primed and expanded with PR3-derived peptides comprising the peptides of Table 141 (Seq. ID. Nos. 1405-1414) and at least one additional set of peptides based on the donor cell source HLA-A profile, wherein the at least one additional set of peptides are selected from the peptides of Tables 142-147. In some embodiments, the PR3-derived peptides also include one or more sets of HLA-B and HLA-DR restricted peptides selected from Tables 148-160 (Seq. ID Nos. 1475-1604).

TABLE 141
Pr3 HLA-A*01 Epitope Peptides

| SEQ ID NO. | Sequence |
|---|---|
| 1405 | FPDFFTRVALY |
| 1406 | GHEAQPHSRPY |
| 1407 | FSVAQVFLNNY |
| 1408 | SVAQVFLNNY |
| 1409 | HEAQPHSRPY |
| 1410 | LRDIPQRLVN |
| 1411 | DFFTRVALY |
| 1412 | EAQPHSRPY |
| 1413 | VAQVFLNNY |
| 1414 | YVDWIRSTLRR |

In some embodiments, the donor cell source is HLA-A*02:01, and the PR3 targeted T-cell subpopulation is primed and expanded with one or more PR3-derived peptides selected from Table 142 (Seq. ID. Nos. 1415-1424). In some embodiments, the donor cell source is HLA-A*02:01, and the PR3 targeted T-cell subpopulation is primed and expanded with PR3-derived peptides selected from Table 142 (Seq. ID. Nos. 1415-1424). In some embodiments, the donor cell source is HLA-A*02:01, and the PR3 targeted T-cell subpopulation is primed and expanded with PR3-derived peptides comprising the peptides of Table 142 (Seq. ID. Nos. 1415-1424). In some embodiments, the donor cell source is HLA-A*02:01, and the PR3 targeted T-cell subpopulation is primed and expanded with PR3-derived peptides comprising the peptides of Table 142 (Seq. ID. Nos. 1415-1424) and at least one additional set of peptides based on the donor cell source HLA-A profile, wherein the at least one additional set of peptides are selected from the peptides of Tables 141, and 143-147. In some embodiments, the PR3-derived peptides also include one or more sets of HLA-B and HLA-DR restricted peptides selected from Tables 148-160 (Seq. ID Nos. 1475-1604).

TABLE 142
Pr3 HLA-A*02:01 Epitope Peptides

| SEQ ID NO. | Sequence |
|---|---|
| 1415 | ALASVLLALL |
| 1416 | KLNDVLLIQL |
| 1417 | VLQELNVTVV |
| 1418 | LICDGIIQGI |
| 1419 | LIHPSFVLTA |
| 1420 | RLFPDFFTRV |
| 1421 | ALYVDWIRST |
| 1422 | NLSASVATV |
| 1423 | LLALLLSGA |
| 1424 | CLAMGWGRV |

In some embodiments, the donor cell source is HLA-A*03, and the PR3 targeted T-cell subpopulation is primed and expanded with one or more PR3-derived peptides selected from Table 143 (Seq. ID. Nos. 1425-1434). In some embodiments, the donor cell source is HLA-A*03, and the PR3 targeted T-cell subpopulation is primed and expanded with PR3-derived peptides selected from Table 143 (Seq. ID. Nos. 1425-1434). In some embodiments, the donor cell source is HLA-A*03, and the PR3 targeted T-cell subpopulation is primed and expanded with PR3-derived peptides comprising the peptides of Table 143 (Seq. ID. Nos. 1425-1434). In some embodiments, the donor cell source is HLA-A*03, and the PR3 targeted T-cell subpopulation is primed and expanded with PR3-derived peptides comprising the peptides of Table 143 (Seq. ID. Nos. 1425-1434) and at least one additional set of peptides based on the donor cell source HLA-A profile, wherein the at least one additional set of peptides are selected from the peptides of Tables 141-142 and 144-147. In some embodiments, the PR3-derived peptides also include one or more sets of HLA-B and HLA-DR restricted peptides selected from Tables 148-160 (Seq. ID Nos. 1475-1604).

TABLE 143
Pr3 HLA-A*03 Epitope Peptides

| SEQ ID NO. | Sequence |
|---|---|
| 1425 | FLNNYDAENK |
| 1426 | TLRRVEAKGR |
| 1427 | QVLQELNVTV |
| 1428 | IVGGHEAQPH |
| 1429 | RLVNVVLGAH |
| 1430 | ALLLSGAAR |
| 1431 | FVIWGCATR |
| 1432 | RLFPDFFTR |
| 1433 | VVLGAHNVR |
| 1434 | ELNVTVVTF |

In some embodiments, the donor cell source is HLA-A*11:01, and the PR3 targeted T-cell subpopulation is primed and expanded with one or more PR3-derived peptides selected from Table 144 (Seq. ID. Nos. 1435-1444). In some embodiments, the donor cell source is HLA-A*11:01, and the PR3 targeted T-cell subpopulation is primed and expanded with PR3-derived peptides selected from Table 144 (Seq. ID. Nos. 1435-1444). In some embodiments, the donor cell source is HLA-A*11:01, and the PR3 targeted T-cell subpopulation is primed and expanded with PR3-derived peptides comprising the peptides of Table 144 (Seq. ID. Nos. 1435-1444). In some embodiments, the donor cell source is HLA-A*11:01, and the PR3 targeted T-cell subpopulation is primed and expanded with PR3-derived peptides comprising the peptides of Table 144 (Seq. ID. Nos. 1435-1444), and at least one additional set of peptides based on the donor cell source HLA-A profile, wherein the at least one additional set of peptides are selected from the peptides of Tables 141-143 and 145-147. In some embodiments, the PR3-derived peptides also include one or more sets of HLA-B and HLA-DR restricted peptides selected from Tables 148-160 (Seq. ID Nos. 1475-1604).

TABLE 144
Pr3 HLA-A*11:01 Epitope Peptides

| SEQ ID NO. | Sequence |
|---|---|
| 1435 | FVLTAAHCLR |
| 1436 | NVTVVTFFCR |
| 1437 | YVDWIRSTLR |
| 1438 | RVALYVDWIR |
| 1439 | STLRRVEAK |
| 1440 | VVLGAHNVR |
| 1441 | ASVLLALLL |
| 1442 | SVLLALLLS |
| 1443 | VTVVTFFCR |
| 1444 | SVAQVFLNN |

In some embodiments, the donor cell source is HLA-A*24:02, and the PR3 targeted T-cell subpopulation is primed and expanded with one or more PR3-derived peptides selected from Table 145 (Seq. ID. Nos. 1445-1454). In some embodiments, the donor cell source is HLA-A*24:02, and the PR3 targeted T-cell subpopulation is primed and expanded with PR3-derived peptides selected from Table 145 (Seq. ID. Nos. 1445-1454). In some embodiments, the donor cell source is HLA-A*24:02, and the PR3 targeted T-cell subpopulation is primed and expanded with PR3-derived peptides comprising the peptides of Table 145 (Seq. ID. Nos. 1445-1454). In some embodiments, the donor cell source is HLA-A*24:02, and the PR3 targeted T-cell subpopulation is primed and expanded with PR3-derived peptides comprising the peptides of Table 145 (Seq. ID. Nos. 1445-1454), and at least one additional set of peptides based on the donor cell source HLA-A profile, wherein the at least one additional set of peptides are selected from the peptides of Tables 141-144 and 146-147. In some embodiments, the PR3-derived peptides also include one or more sets of HLA-B and HLA-DR restricted peptides selected from Tables 148-160 (Seq. ID Nos. 1475-1604).

TABLE 145
Pr3 HLA-A*24:02 Epitope Peptides

| SEQ ID NO. | Sequence |
|---|---|
| 1445 | LYVDWIRSTL |
| 1446 | CFGDSGGPLI |
| 1447 | TFVPRRKAGI |
| 1448 | SFVLTAAHCL |
| 1449 | PSPALASVLL |
| 1450 | VIWGCATRLF |
| 1451 | TFFCRPHNI |
| 1452 | CFGDSGGPL |
| 1453 | HFSVAQVFL |
| 1454 | NKLNDVLLI |

In some embodiments, the donor cell source is HLA-A*26, and the PR3 targeted T-cell subpopulation is primed and expanded with one or more PR3-derived peptides selected from Table 146 (Seq. ID. Nos. 1455-1464). In some embodiments, the donor cell source is HLA-A*26, and the PR3 targeted T-cell subpopulation is primed and expanded with PR3-derived peptides selected from Table 146 (Seq. ID. Nos. 1455-1464). In some embodiments, the donor cell source is HLA-A*26, and the PR3 targeted T-cell subpopulation is primed and expanded with PR3-derived peptides comprising the peptides of Table 146 (Seq. ID. Nos. 1455-1464). In some embodiments, the donor cell source is HLA-A*26, and the PR3 targeted T-cell subpopulation is primed and expanded with PR3-derived peptides comprising the peptides of Table 146 (Seq. ID. Nos. 1455-1464) and at least one additional set of peptides based on the donor cell source HLA-A profile, wherein the at least one additional set of peptides are selected from the peptides of Tables 141-145 and 147. In some embodiments, the PR3-derived peptides also include one or more sets of HLA-B and HLA-DR restricted peptides selected from Tables 148-160 (Seq. ID Nos. 1475-1604).

TABLE 146
Pr3 HLA-A*26 Epitope Peptides

| SEQ ID NO. | Sequence |
|---|---|
| 1455 | SVAQVFLNNY |
| 1456 | DGIIQGIDSF |
| 1457 | DVLLIQLSSP |
| 1458 | ELNVTVVTFF |
| 1459 | FVIWGCATRL |
| 1460 | DFFTRVALY |
| 1461 | ELNVTVVTF |
| 1462 | DVLLIQLSS |
| 1463 | YVDWIRSTL |
| 1464 | EAQPHSRPY |

In some embodiments, the donor cell source is HLA-A*68:01, and the PR3 targeted T-cell subpopulation is primed and expanded with one or more PR3-derived peptides selected from Table 147 (Seq. ID. Nos. 1465-1474). In some embodiments, the donor cell source is HLA-A*68:01, and the PR3 targeted T-cell subpopulation is primed and expanded with PR3-derived peptides selected from Table 147 (Seq. ID. Nos. 1465-1474). In some embodiments, the donor cell source is HLA-A*68:01, and the PR3 targeted T-cell subpopulation is primed and expanded with PR3-derived peptides comprising the peptides of Table 147 (Seq. ID. Nos. 1465-1474). In some embodiments, the donor cell source is HLA-A*68:01, and the PR3 targeted T-cell subpopulation is primed and expanded with PR3-derived peptides comprising the peptides of Table 147 (Seq. ID. Nos. 1465-1474), and at least one additional set of peptides based on the donor cell source HLA-A profile, wherein the at least one additional set of peptides are selected from the peptides of Tables 141-146. In some embodiments, the PR3-derived peptides also include one or more sets of HLA-B and HLA-DR restricted peptides selected from Tables 148-160 (Seq. ID Nos. 1475-1604).

TABLE 147
Pr3 HLA-A*68:01 Epitope Peptides

| SEQ ID NO. | Sequence |
|---|---|
| 1465 | DSFVIWGCATR |
| 1466 | LLALLLSGAAR |
| 1467 | GTQCLAMGWGR |
| 1468 | STLRRVEAKGR |
| 1469 | LALLLSGAAR |
| 1470 | YVDWIRSTLR |
| 1471 | NVVLGAHNVR |
| 1472 | NVTVVTFFCR |
| 1473 | RVALYVDWIR |
| 1474 | ATRLFPDFFTR |

In some embodiments, the donor cell source is HLA-B*07:02, and the PR3 targeted T-cell subpopulation is primed and expanded with one or more PR3-derived peptides selected from Table 148 (Seq. ID. Nos. 1475-1484). In some embodiments, the donor cell source is HLA-B*07:02, and the PR3 targeted T-cell subpopulation is primed and expanded with PR3-derived peptides selected from Table 148 (Seq. ID. Nos. 1475-1484). In some embodiments, the donor cell source is HLA-B*07:02, and the PR3 targeted T-cell subpopulation is primed and expanded with PR3-derived peptides comprising the peptides of Table 148 (Seq. ID. Nos. 1475-1484). In some embodiments, the donor cell source is HLA-B*07:02, and the PR3 targeted T-cell subpopulation is primed and expanded with PR3-derived peptides comprising the peptides of Table 148 (Seq. ID. Nos. 1475-1484), and at least one additional set of peptides based on the donor cell source HLA-B profile, wherein the at least one additional set of peptides are selected from the peptides of Tables 149-154. In some embodiments, the PR3-derived peptides also include one or more sets of HLA-A and HLA-DR restricted peptides selected from Tables 141-147 and 155-160 (Seq. ID Nos. 1405-1474 and 1545-1604).

TABLE 148
Pr3 HLA-B*07:02 Epitope Peptides

| SEQ ID NO. | Sequence |
|---|---|
| 1475 | FPDFFTRVAL |
| 1476 | PPSPALASVL |
| 1477 | IPQRLVNVVL |
| 1478 | QPVPHGTQCL |
| 1479 | VPHGTQCLAM |
| 1480 | DPPAQVLQEL |
| 1481 | SPALASVLL |
| 1482 | PPAQVLQEL |
| 1483 | AHRPPSPAL |
| 1484 | HPSFVLTAA |

In some embodiments, the donor cell source is HLA-B*08, and the PR3 targeted T-cell subpopulation is primed and expanded with one or more PR3-derived peptides selected from Table 149 (Seq. ID. Nos. 1485-1494). In some embodiments, the donor cell source is HLA-B*08, and the PR3 targeted T-cell subpopulation is primed and expanded with PR3-derived peptides selected from Table 149 (Seq. ID. Nos. 1485-1494). In some embodiments, the donor cell source is HLA-B*08, and the PR3 targeted T-cell subpopulation is primed and expanded with PR3-derived peptides comprising the peptides of Table 149 (Seq. ID. Nos. 1485-1494). In some embodiments, the donor cell source is HLA-B*08, and the PR3 targeted T-cell subpopulation is primed and expanded with PR3-derived peptides comprising the peptides of Table 149 (Seq. ID. Nos. 1485-1494) and at least one additional set of peptides based on the donor cell source HLA-B profile, wherein the at least one additional set of peptides are selected from the peptides of Tables 148 and 150-154. In some embodiments, the PR3-derived peptides also include one or more sets of HLA-A and HLA-DR restricted peptides selected from Tables 141-147 and 155-160 (Seq. ID Nos. 1405-1474 and 1545-1604).

TABLE 149
Pr3 HLA-B*08 Epitope Peptides

| SEQ ID NO. | Sequence |
|---|---|
| 1485 | ENKLNDVLL |
| 1486 | VPRRKAGIC |
| 1487 | CLRDIPQRL |
| 1488 | SPALASVLL |
| 1489 | PQRLVNVVL |
| 1490 | SASVATVQL |
| 1491 | VPRRKAGI |
| 1492 | ENKLNDVL |
| 1493 | VDWIRSTL |
| 1494 | DFFTRVAL |

In some embodiments, the donor cell source is HLA-B*15:01, and the PR3 targeted T-cell subpopulation is primed and expanded with one or more PR3-derived peptides selected from Table 150 (Seq. ID. Nos. 1495-1504). In some embodiments, the donor cell source is HLA-B*15:01, and the PR3 targeted T-cell subpopulation is primed and expanded with PR3-derived peptides selected from Table 150 (Seq. ID. Nos. 1495-1504). In some embodiments, the donor cell source is HLA-B*15:01, and the PR3 targeted T-cell subpopulation is primed and expanded with PR3-derived peptides comprising the peptides of Table 150 (Seq. ID. Nos. 1495-1504). In some embodiments, the donor cell source is HLA-B*15:01, and the PR3 targeted T-cell subpopulation is primed and expanded with PR3-derived peptides comprising the peptides of Table 150 (Seq. ID. Nos. 1495-1504) and at least one additional set of peptides based on the donor cell source HLA-B profile, wherein the at least one additional set of peptides are selected from the peptides of Tables 148-149 and 151-154. In some embodiments, the PR3-derived peptides also include one or more sets of HLA-A and HLA-DR restricted peptides selected from Tables 141-147 and 155-160 (Seq. ID Nos. 1405-1474 and 1545-1604).

TABLE 150
Pr3 HLA-B*15:01 (B62) Epitope Peptides

| SEQ ID NO. | Sequence |
|---|---|
| 1495 | QQHFSVAQVF |
| 1496 | SVAQVFLNNY |
| 1497 | ELNVTVVTFF |
| 1498 | RLFPDFFTRV |
| 1499 | RLVNVVLGAH |
| 1500 | KLNDVLLIQL |
| 1501 | ELNVTVVTF |
| 1502 | TQEPTQQHF |
| 1503 | GIIQGIDSF |
| 1504 | ALASVLLAL |

In some embodiments, the donor cell source is HLA-B*18, and the PR3 targeted T-cell subpopulation is primed and expanded with one or more PR3-derived peptides selected from Table 151 (Seq. ID. Nos. 1505-1514). In some embodiments, the donor cell source is HLA-B*18, and the PR3 targeted T-cell subpopulation is primed and expanded with PR3-derived peptides selected from Table 151 (Seq. ID. Nos. 1505-1514). In some embodiments, the donor cell source is HLA-B*18, and the PR3 targeted T-cell subpopulation is primed and expanded with PR3-derived peptides comprising the peptides of Table 151 (Seq. ID. Nos. 1505-1514). In some embodiments, the donor cell source is HLA-B*18, and the PR3 targeted T-cell subpopulation is primed and expanded with PR3-derived peptides comprising the peptides of Table 151 (Seq. ID. Nos. 1505-1514) and at least one additional set of peptides based on the donor cell source HLA-B profile, wherein the at least one additional set of peptides are selected from the peptides of Tables 148-150 and 152-154. In some embodiments, the PR3-derived peptides also include one or more sets of HLA-A and HLA-DR restricted peptides selected from Tables 141-147 and 155-160 (Seq. ID Nos. 1405-1474 and 1545-1604).

TABLE 151
Pr3 HLA-B*18 Epitope Peptides

| SEQ ID NO. | Sequence |
|---|---|
| 1505 | AENKLNDVL |
| 1506 | DFFTRVALY |
| 1507 | ATRLFPDFF |
| 1508 | GGTLIHPSF |
| 1509 | QEPTQQHF |
| 1510 | LNVTVVTF |
| 1511 | QELNVTVV |
| 1512 | NDVLLIQL |
| 1513 | PRRKAGICF |
| 1514 | CATRLFPDF |

In some embodiments, the donor cell source is HLA-B*27:05, and the PR3 targeted T-cell subpopulation is primed and expanded with one or more PR3-derived peptides selected from Table 152 (Seq. ID. Nos. 1515-1524). In some embodiments, the donor cell source is HLA-B*27:05, and the PR3 targeted T-cell subpopulation is primed and expanded with PR3-derived peptides selected from Table 152 (Seq. ID. Nos. 1515-1524). In some embodiments, the donor cell source is HLA-B*27:05, and the PR3 targeted T-cell subpopulation is primed and expanded with PR3-derived peptides comprising the peptides of Table 152 (Seq. ID. Nos. 1515-1524). In some embodiments, the donor cell source is HLA-B*27:05, and the PR3 targeted T-cell subpopulation is primed and expanded with PR3-derived peptides comprising the peptides of Table 152 (Seq. ID. Nos. 1515-1524) and at least one additional set of peptides based on the donor cell source HLA-B profile, wherein the at least one additional set of peptides are selected from the peptides of Tables 148-151 and 153-154. In some embodiments, the PR3-derived peptides also include one or more sets of HLA-A and HLA-DR restricted peptides selected from Tables 141-147 and 155-160 (Seq. ID Nos. 1405-1474 and 1545-1604).

TABLE 152
Pr3 HLA-B*27:05 Epitope Peptides

| SEQ ID NO. | Sequence |
|---|---|
| 1515 | TRLFPDFFTR |
| 1516 | SRPYMASLQM |
| 1517 | VRTQEPTQQH |
| 1518 | ARAAEIVGGH |
| 1519 | CRPHNICTF |
| 1520 | PRRKAGICF |
| 1521 | GIIQGIDSF |
| 1522 | MRGNPGSHF |
| 1523 | LRRVEAKGR |
| 1524 | RRVEAKGRP |

In some embodiments, the donor cell source is HLA-B*35:01, and the PR3 targeted T-cell subpopulation is primed and expanded with one or more PR3-derived peptides selected from Table 153 (Seq. ID. Nos. 1525-1534). In some embodiments, the donor cell source is HLA-B*35:01, and the PR3 targeted T-cell subpopulation is primed and expanded with PR3-derived peptides selected from Table 153 (Seq. ID. Nos. 1525-1534). In some embodiments, the donor cell source is HLA-B*35:01, and the PR3 targeted T-cell subpopulation is primed and expanded with PR3-derived peptides comprising the peptides of Table 153 (Seq. ID. Nos. 1525-1534). In some embodiments, the donor cell source is HLA-B*35:01, and the PR3 targeted T-cell subpopulation is primed and expanded with PR3-derived peptides comprising the peptides of Table 153 (Seq. ID. Nos. 1525-1534) and at least one additional set of peptides based on the donor cell source HLA-B profile, wherein the at least one additional set of peptides are selected from the peptides of Tables 148-152 and 154. In some embodiments, the PR3-derived peptides also include one or more sets of HLA-A and HLA-DR restricted peptides selected from Tables 141-147 and 155-160 (Seq. ID Nos. 1405-1474 and 1545-1604).

TABLE 153
Pr3 HLA-B*35:01 Epitope Peptides

| SEQ ID NO. | Sequence |
|---|---|
| 1525 | FPDFFTRVAL |
| 1526 | IPQRLVNVVL |
| 1527 | PPSPALASVL |
| 1528 | DPPAQVLQEL |
| 1529 | QPVPHGTQCL |
| 1530 | VPRRKAGICF |
| 1531 | SPALASVLL |
| 1532 | PPAQVLQEL |
| 1533 | GPLICDGII |
| 1534 | RPYMASLQM |

In some embodiments, the donor cell source is HLA-B*58:02, and the PR3 targeted T-cell subpopulation is primed and expanded with one or more PR3-derived peptides selected from Table 154 (Seq. ID. Nos. 1535-1544). In some embodiments, the donor cell source is HLA-B*58:02, and the PR3 targeted T-cell subpopulation is primed and expanded with PR3-derived peptides selected from Table 154 (Seq. ID. Nos. 1535-1544). In some embodiments, the donor cell source is HLA-B*58:02, and the PR3 targeted T-cell subpopulation is primed and expanded with PR3-derived peptides comprising the peptides of Table 154 (Seq. ID. Nos. 1535-1544). In some embodiments, the donor cell source is HLA-B*58:02, and the PR3 targeted T-cell subpopulation is primed and expanded with PR3-derived peptides comprising the peptides of Table 154 (Seq. ID. Nos. 1535-1544) and at least one additional set of peptides based on the donor cell source HLA-B profile, wherein the at least one additional set of peptides are selected from the peptides of Tables 148-153. In some embodiments, the PR3-derived peptides also include one or more sets of HLA-A and HLA-DR restricted peptides selected from Tables 141-147 and 155-160 (Seq. ID Nos. 1405-1474 and 1545-1604).

TABLE 154
Pr3 HLA-B*58:02 Epitope Peptides

| SEQ ID NO. | Sequence |
|---|---|
| 1535 | RTQEPTQQHF |
| 1536 | LTAAHCLRDI |
| 1537 | PSPALASVLL |
| 1538 | LSGAARAAEI |
| 1539 | GSHFCGGTLI |
| 1540 | ASVLLALLL |
| 1541 | EAQPHSRPY |
| 1542 | HSRPYMASL |
| 1543 | RVALYVDWI |
| 1544 | CATRLFPDF |

In some embodiments, the donor cell source is HLA-DRB1*0101, and the PR3 targeted T-cell subpopulation is primed and expanded with one or more PR3-derived peptides selected from Table 155 (Seq. ID. Nos. 1545-1554). In some embodiments, the donor cell source is HLA-DRB1*0101, and the PR3 targeted T-cell subpopulation is primed and expanded with PR3-derived peptides selected from Table 155 (Seq. ID. Nos. 1545-1554). In some embodiments, the donor cell source is HLA-DRB1*0101, and the PR3 targeted T-cell subpopulation is primed and expanded with PR3-derived peptides comprising the peptides of Table 155 (Seq. ID. Nos. 1545-1554). In some embodiments, the donor cell source is HLA-DRB1*0101, and the PR3 targeted T-cell subpopulation is primed and expanded with PR3-derived peptides comprising the peptides of Table 155 (Seq. ID. Nos. 1545-1554) and at least one additional set of peptides based on the donor cell source HLA-DR profile, wherein the at least one additional set of peptides are selected from the peptides of Tables 156-160. In some embodiments, the PR3-derived peptides also include one or more sets of HLA-A and HLA-B restricted peptides selected from Tables 141-154 (Seq. ID Nos. 1405-1544).

TABLE 155
Pr3 HLA-DRB1*0101 Epitope Peptides

| SEQ ID NO. | Sequence |
|---|---|
| 1545 | AMGWGRVGAHDPPAQ |
| 1546 | SVLLALLLSGAARAA |
| 1547 | NDVLLIQLSSPANLS |
| 1548 | HPSFVLTAAHCLRDI |
| 1549 | DGIIQGIDSFVIWGC |
| 1550 | GICFGDSGGPLICDG |
| 1551 | ASVLLALLLSGAARA |
| 1552 | LLALLLSGAARAAEI |
| 1553 | ARAAEIVGGHEAQPH |
| 1554 | SLQMRGNPGSHFCGG |

In some embodiments, the donor cell source is HLA-DRB1*0301, and the PR3 targeted T-cell subpopulation is primed and expanded with one or more PR3-derived peptides selected from Table 156 (Seq. ID. Nos. 1555-1564). In some embodiments, the donor cell source is HLA-DRB1*0301, and the PR3 targeted T-cell subpopulation is primed and expanded with PR3-derived peptides selected from Table 156 (Seq. ID. Nos. 1555-1564). In some embodiments, the donor cell source is HLA-DRB1*0301, and the PR3 targeted T-cell subpopulation is primed and expanded with PR3-derived peptides comprising the peptides of Table 156 (Seq. ID. Nos. 1555-1564). In some embodiments, the donor cell source is HLA-DRB1*0301, and the PR3 targeted T-cell subpopulation is primed and expanded with PR3-derived peptides comprising the peptides of Table 156 (Seq. ID. Nos. 1555-1564) and at least one additional set of peptides based on the donor cell source HLA-DR profile, wherein the at least one additional set of peptides are selected from the peptides of Tables 155 and 157-160. In some embodiments, the PR3-derived peptides also include one or more sets of HLA-A and HLA-B restricted peptides selected from Tables 141-154 (Seq. ID Nos. 1405-1544).

TABLE 156
Pr3 HLA-DRB1*0301 (DR17) Epitope Peptides

| SEQ ID NO. | Sequence |
|---|---|
| 1555 | VFLNNYDAENKLNDV |
| 1556 | SPALASVLLALLLSG |
| 1557 | VLLIQLSSPANLSAS |
| 1558 | GGPLICDGIIQGIDS |
| 1559 | AAHCLRDIPQRLVNV |
| 1560 | ATRLFPDFFTRVALY |
| 1561 | SLQMRGNPGSHFCGG |
| 1562 | HFSVAQVFLNNYDAE |
| 1563 | PAQVLQELNVTVVTF |
| 1564 | RVALYVDWIRSTLRR |

In some embodiments, the donor cell source is HLA-DRB1*0401, and the PR3 targeted T-cell subpopulation is primed and expanded with one or more PR3-derived peptides selected from Table 157 (Seq. ID. Nos. 1565-1574). In some embodiments, the donor cell source is HLA-DRB 1*0401, and the PR3 targeted T-cell subpopulation is primed and expanded with PR3-derived peptides selected from Table 157 (Seq. ID. Nos. 1565-1574). In some embodiments, the donor cell source is HLA-DRB1*0401, and the PR3 targeted T-cell subpopulation is primed and expanded with PR3-derived peptides comprising the peptides of Table 157 (Seq. ID. Nos. 1565-1574). In some embodiments, the donor cell source is HLA-DRB1*0401, and the PR3 targeted T-cell subpopulation is primed and expanded with PR3-derived peptides comprising the peptides of Table 157 (Seq. ID. Nos. 1565-1574) and at least one additional set of peptides based on the donor cell source HLA-DR profile, wherein the at least one additional set of peptides are selected from the peptides of Tables 155-156 and 158-160. In some embodiments, the PR3-derived peptides also include one or more sets of HLA-A and HLA-B restricted peptides selected from Tables 141-154 (Seq. ID Nos. 1405-1544).

TABLE 157
Pr3 HLA-DRB1*0401 (DR4Dw4) Epitope Peptides

| SEQ ID NO. | Sequence |
|---|---|
| 1565 | TRLFPDFFTRVALYV |
| 1566 | SVLLALLLSGAARAA |
| 1567 | AHCLRDIPQRLVNVV |
| 1568 | VNVVLGAHNVRTQEP |
| 1569 | DVLLIQLSSPANLSA |
| 1570 | PAQVLQELNVTVVTF |
| 1571 | VTVVTFFCRPHNICT |
| 1572 | DGIIQGIDSPVIWGC |
| 1573 | QQHFSVAQVFLNNYD |
| 1574 | VTFFCRPHNICTFVP |

In some embodiments, the donor cell source is HLA-DRB1*0701, and the PR3 targeted T-cell subpopulation is primed and expanded with one or more PR3-derived peptides selected from Table 158 (Seq. ID. Nos. 1575-1584). In some embodiments, the donor cell source is HLA-DRB 1*0701, and the PR3 targeted T-cell subpopulation is primed and expanded with PR3-derived peptides selected from Table 158 (Seq. ID. Nos. 1575-1584). In some embodiments, the donor cell source is HLA-DRB1*0701, and the PR3 targeted T-cell subpopulation is primed and expanded with PR3-derived peptides comprising the peptides of Table 158 (Seq. ID. Nos. 1575-1584). In some embodiments, the donor cell source is HLA-DRB1*0701, and the PR3 targeted T-cell subpopulation is primed and expanded with PR3-derived peptides comprising the peptides of Table 158 (Seq. ID. Nos. 1575-1584) and at least one additional set of peptides based on the donor cell source HLA-DR profile, wherein the at least one additional set of peptides are selected from the peptides of Tables 155-157 and 159-160. In some embodiments, the PR3-derived peptides also include one or more sets of HLA-A and HLA-B restricted peptides selected from Tables 141-154 (Seq. ID Nos. 1405-1544).

TABLE 158
Pr3 HLA-DRB1*0701 Epitope Peptides

| SEQ ID NO. | Sequence |
|---|---|
| 1575 | DGIIQGIDSFVIWGC |
| 1576 | HPSFVLTAAHCLRDI |
| 1577 | GICFGDSGGPLICDG |
| 1578 | YVDWIRSTLRRVEAK |
| 1579 | AHCLRDIPQRLVNVV |
| 1580 | VLLIQLSSPANLSAS |
| 1581 | PAQVLQELNVTVVTF |
| 1582 | LQELNVTVVTFFCRP |
| 1583 | CDGIIQGIDSFVIWG |
| 1584 | SFVIWGCATRLFPDF |

In some embodiments, the donor cell source is HLA-DRB1*1101, and the PR3 targeted T-cell subpopulation is primed and expanded with one or more PR3-derived peptides selected from Table 159 (Seq. ID. Nos. 1585-1594). In some embodiments, the donor cell source is HLA-DRB 1*1101, and the PR3 targeted T-cell subpopulation is primed and expanded with PR3-derived peptides selected from Table 159 (Seq. ID. Nos. 1585-1594). In some embodiments, the donor cell source is HLA-DRB1*1101, and the PR3 targeted T-cell subpopulation is primed and expanded with PR3-derived peptides comprising the peptides of Table 159 (Seq. ID. Nos. 1585-1594). In some embodiments, the donor cell source is HLA-DRB1*1101, and the PR3 targeted T-cell subpopulation is primed and expanded with PR3-derived peptides comprising the peptides of Table 159 (Seq. ID. Nos. 1585-1594) and at least one additional set of peptides based on the donor cell source HLA-DR profile, wherein the at least one additional set of peptides are selected from the peptides of Tables 155-158 and 160. In some embodiments, the PR3-derived peptides also include one or more sets of HLA-A and HLA-B restricted peptides selected from Tables 141-154 (Seq. ID Nos. 1405-1544).

TABLE 159
Pr3 HLA-DRB1*1101 Epitope Peptides

| SEQ ID NO. | Sequence |
|---|---|
| 1585 | HNICTFVPRRKAGIC |
| 1586 | FVIWGCATRLFPDFF |
| 1587 | AMGWGRVGAHDPPAQ |
| 1588 | VVTFFCRPHNICTFV |
| 1589 | DWIRSTLRRVEAKGR |
| 1590 | PDFFTRVALYVDWIR |
| 1591 | ASVLLALLLSGAARA |
| 1592 | SVLLALLLSGAARAA |
| 1593 | SRPYMASLQMRGNPG |
| 1594 | LNNYDAENKLNDVLL |

In some embodiments, the donor cell source is HLA-DRB1*1501, and the PR3 targeted T-cell subpopulation is primed and expanded with one or more PR3-derived peptides selected from Table 160 (Seq. ID. Nos. 1595-1604). In some embodiments, the donor cell source is HLA-DRB1*1501, and the PR3 targeted T-cell subpopulation is primed and expanded with PR3-derived peptides selected from Table 160 (Seq. ID. Nos. 1595-1604). In some embodiments, the donor cell source is HLA-DRB1*1501, and the PR3 targeted T-cell subpopulation is primed and expanded with PR3-derived peptides comprising the peptides of Table 160 (Seq. ID. Nos. 1595-1604). In some embodiments, the donor cell source is HLA-DRB1*1501, and the PR3 targeted T-cell subpopulation is primed and expanded with PR3-derived peptides comprising the peptides of Table 160 (Seq. ID. Nos. 1595-1604) and at least one additional set of peptides based on the donor cell source HLA-DR profile, wherein the at least one additional set of peptides are selected from the peptides of Tables 155-159. In some embodiments, the PR3-derived peptides also include one or more sets of HLA-A and HLA-B restricted peptides selected from Tables 141-154 (Seq. ID Nos. 1405-1544).

TABLE 160
Pr3 HLA-DRB1*1501 (DR2b) Epitope Peptides

| SEQ ID NO. | Sequence |
|---|---|
| 1595 | QVFLNNYDAENKLND |
| 1596 | PHNICTFVPRRKAGI |
| 1597 | TRLFPDFFTRVALYV |
| 1598 | VNVVLGAHNVRTQEP |
| 1599 | LIQLSSPANLSASVA |
| 1600 | PAQVLQELNVTVVTF |
| 1601 | NVTVVTFFCRPHNIC |
| 1602 | VTVVTFFCRPHNICT |
| 1603 | DGIIQGIDSFVIWGC |
| 1604 | IQGIDSFVIWGCATR |

Cyclin-A₁ Antigenic Peptides

In some embodiments, the MUSTANG composition includes Cyclin-A₁ specific T-cells. Cyclin-A₁ specific T-cells can be generated as described below using one or more antigenic peptides to Cyclin-A₁. In some embodiments, the Cyclin-A₁ specific T-cells are generated using one or more antigenic peptides to Cyclin-A₁, or a modified or heteroclitic peptide derived from a Cyclin-A₁ peptide. In some embodiments, Cyclin-A₁ specific T-cells are generated using a Cyclin-A₁ antigen library comprising a pool of peptides (for example 15mers) containing amino acid overlap (for example 11 amino acids of overlap) between each sequence formed by scanning the protein amino acid sequence SEQ. ID. No. 1605 (UniProt KB-P78396) for Cyclin-A₁:

METGFPAIMYPGSFIGGWGEEYLSWEGPGLPDFVFQQQPVESEAMHCSNPK
SGVVLATVARGPDACQILTRAPLGQDPPQRTVLGLLTANGQYRRTCGQGIT
RIRCYSGSENAFPPAGKKALPDCGVQEPPKQGFDIYMDELEQGDRDSCSVR
EGMAFEDVYEVDTGTLKSDLHFLLDFNTVSPMLVDSSLLSQSEDISSLGTD
VINVTEYAEEIYQYLREAEIRHRPKAHYMKKQPDITEGMRTILVDWLVEVG
EEYKLRAETLYLAVNFLDRFLSCMSVLRGKLQLVGTAAMLLASKYEEIYPP
EVDEFVYITDDTYTKRQLLKMEHLLLKVLAFDLTVPTTNQFLLQYLRRQGV
CVRTENLAKYVAELSLLEADPFLKYLPSLIAAAAFCLANYTVNKHFWPETL
AAFTGYSLSEIVPCLSELHKAYLDIPHRPQQAIREKYKASKYLCVSLMEPP
AVLLLQ.

In some embodiments, the Cyclin-A₁ specific T-cells are generated using one or more antigenic peptides to Cyclin-A₁, or a modified or heteroclitic peptide derived from a Cyclin-A₁ peptide. In some embodiments, the Cyclin-A₁ specific T-cells are generated with peptides that recognize class I MEW molecules. In some embodiments, the Cyclin-A₁ specific T-cells are generated with peptides that recognize class II MHC molecules. In some embodiments, the Cyclin-A₁ specific T-cells are generated with peptides that recognize both class I and class II MEW molecules.

In some embodiments, the Cyclin-A₁ peptides used to prime and expand a T-cell subpopulation includes specifically selected HLA-restricted peptides generated by determining the HLA profile of the donor source, and including peptides derived from Cyclin-A₁ that best match the donor's HLA. In some embodiments, the Cyclin-A₁ peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting Cyclin-A₁ derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 161-167, the HLA-B peptides are selected from the peptides of Tables 168-174, and the HLA-DR peptides are selected from the peptides of Tables 175-180. For example, if the donor cell source has an HLA profile that is HLA-A*01/*02:01; HLA-B*15:01/*18; and HLA-DRB1*0101/*0301, then the Cyclin-A₁ peptides used to prime and expand the Cyclin-A₁ specific T-cell subpopulation are restricted to the specific HLA profile, and may include the peptides identified in Table 161 (Seq. ID. Nos. 1606-1616) for HLA-A*01; Table 162 (Seq. ID. Nos. 1617-1626) for HLA-A*02:01; Table 170 (Seq. ID. Nos. 1698-1707) for HLA-B*15:01; Table 171 (Seq. ID. Nos. 1708-1717) for HLA-B*18; Table 175 (Seq. ID. Nos. 1747-1756) for HLA-DRB1*0101; and Table 176 (Seq. ID. Nos. 1757-1766) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the Cyclin-A₁ targeted T-cell subpopulation is primed and expanded with one or more Cyclin-A₁-derived peptides selected from Table 161 (Seq. ID. Nos. 1606-1616). In some embodiments, the donor cell source is HLA-A*01, and the Cyclin-A₁ targeted T-cell subpopulation is primed and expanded with Cyclin-A₁-derived peptides selected from Table 161 (Seq. ID. Nos. 1606-1616). In some embodiments, the donor cell source is HLA-A*01, and the Cyclin-A₁ targeted T-cell subpopulation is primed and expanded with Cyclin-A₁-derived peptides comprising the peptides of Table 161 (Seq. ID. Nos. 1606-1616). In some embodiments, the donor cell source is HLA-A*01, and the Cyclin-A₁ targeted T-cell subpopulation is primed and expanded with Cyclin-A₁-derived peptides comprising the peptides of Table 161 (Seq. ID. Nos. 1606-1616) and at least one additional set of peptides based on the donor cell source HLA-A profile, wherein the at least one additional set of peptides are selected from the peptides of Tables 162-167. In some embodiments, the Cyclin-A₁-derived peptides also include one or more sets of HLA-B and HLA-DR restricted peptides selected from Tables 168-180 (Seq. ID Nos. 1678-1806).

TABLE 161
Cyclin A₁ HLA-A*01 Epitope Peptides

| SEQ ID NO. | Sequence |
|---|---|
| 1606 | VTEYAEEIYQY |
| 1607 | VREGMAFEDVY |
| 1608 | WPETLAAFTGY |
| 1609 | GTAAMLLASKY |
| 1610 | GTDVINVTEY |
| 1611 | LLEADPFLKY |
| 1612 | PTTNQFLLQY |
| 1613 | IREKYKASKY |
| 1614 | TTNQFLLQY |
| 1615 | PPEVDEFVY |
| 1616 | AAMLLASKY |

In some embodiments, the donor cell source is HLA-A*02:01, and the Cyclin-A₁ targeted T-cell subpopulation is primed and expanded with one or more Cyclin-A₁-derived peptides selected from Table 162 (Seq. ID. Nos. 1617-1626). In some embodiments, the donor cell source is HLA-A*02:01, and the Cyclin-A₁ targeted T-cell subpopulation is primed and expanded with Cyclin-A₁-derived peptides selected from Table 162 (Seq. ID. Nos. 1617-1626). In some embodiments, the donor cell source is HLA-A*02:01, and the Cyclin-A₁ targeted T-cell subpopulation is primed and expanded with Cyclin-A₁-derived peptides comprising the peptides of Table 162 (Seq. ID. Nos. 1617-1626). In some embodiments, the donor cell source is HLA-A*02:01, and the Cyclin-A₁ targeted T-cell subpopulation is primed and expanded with Cyclin-A₁-derived peptides comprising the peptides of Table 162 (Seq. ID. Nos. 1617-1626) and at least one additional set of peptides based on the donor cell source HLA-A profile, wherein the at least one additional set of peptides are selected from the peptides of Tables 161, and 163-167. In some embodiments, the Cyclin-A₁-derived peptides also include one or more sets of HLA-B and HLA-DR restricted peptides selected from Tables 168-180 (Seq. ID Nos. 1678-1806).

TABLE 162
Cyclin A₁ HLA-A*02:01 Epitope Peptides

| SEQ ID NO. | Sequence |
|---|---|
| 1617 | TILVDWLVEV |
| 1618 | GMAFEDVYEV |
| 1619 | SLMEPPAVLL |
| 1620 | NLAKYVAEL |
| 1621 | SLSEIVPCL |
| 1622 | VLRGKLQLV |
| 1623 | SLLEADPFL |
| 1624 | SLGTDVINV |
| 1625 | TLYLAVNFL |
| 1626 | KMEHLLLKV |

In some embodiments, the donor cell source is HLA-A*03, and the Cyclin-A₁ targeted T-cell subpopulation is primed and expanded with one or more Cyclin-A₁-derived peptides selected from Table 163 (Seq. ID. Nos. 1627-1637). In some embodiments, the donor cell source is HLA-A*03, and the Cyclin-A₁ targeted T-cell subpopulation is primed and expanded with Cyclin-A₁-derived peptides selected from Table 163 (Seq. ID. Nos. 1627-1637). In some embodiments, the donor cell source is HLA-A*03, and the Cyclin-A₁ targeted T-cell subpopulation is primed and expanded with Cyclin-A₁-derived peptides comprising the peptides of Table 163 (Seq. ID. Nos. 1627-1637). In some embodiments, the donor cell source is HLA-A*03, and the Cyclin-A₁ targeted T-cell subpopulation is primed and expanded with Cyclin-A₁-derived peptides comprising the peptides of Table 163 (Seq. ID. Nos. 1627-1637) and at least one additional set of peptides based on the donor cell source HLA-A profile, wherein the at least one additional set of peptides are selected from the peptides of Tables 161-162 and 164-167. In some embodiments, the Cyclin-A₁-derived peptides also include one or more sets of HLA-B and HLA-DR restricted peptides selected from Tables 168-180 (Seq. ID Nos. 1678-1806).

TABLE 163
Cyclin A₁ HLA-A*03 Epitope Peptides

| SEQ ID NO. | Sequence |
|---|---|
| 1627 | AIREKYKASK |
| 1628 | SLLEADPFLK |
| 1629 | IVPCLSELHK |
| 1630 | LLKMEHLLLK |
| 1631 | YLRRQGVCVR |
| 1632 | CVRTENLAK |
| 1633 | CLANYTVNK |
| 1634 | FVYITDDTY |
| 1635 | ALPDCGVQE |
| 1636 | FIGGWGEEY |
| 1637 | SVLRGKLQL |

In some embodiments, the donor cell source is HLA-A*11:01, and the Cyclin-A₁ targeted T-cell subpopulation is primed and expanded with one or more Cyclin-A₁-derived peptides selected from Table 164 (Seq. ID. Nos. 1638-1647). In some embodiments, the donor cell source is HLA-A*11:01, and the Cyclin-A₁ targeted T-cell subpopulation is primed and expanded with Cyclin-A₁-derived peptides selected from Table 164 (Seq. ID. Nos. 1638-1647). In some embodiments, the donor cell source is HLA-A*11:01, and the Cyclin-A₁ targeted T-cell subpopulation is primed and expanded with Cyclin-A₁-derived peptides comprising the peptides of Table 164 (Seq. ID. Nos. 1638-1647). In some embodiments, the donor cell source is HLA-A*11:01, and the Cyclin-A₁ targeted T-cell subpopulation is primed and expanded with Cyclin-A₁-derived peptides comprising the peptides of Table 164 (Seq. ID. Nos. 1638-1647), and at least one additional set of peptides based on the donor cell source HLA-A profile, wherein the at least one additional set of peptides are selected from the peptides of Tables 141-143 and 145-147. In some embodiments, the Cyclin-A₁-derived peptides also include one or more sets of HLA-B and HLA-DR restricted peptides selected from Tables 168-180 (Seq. ID Nos. 1678-1806).

TABLE 164
Cyclin A₁ HLA-A*11:01 Epitope Peptides

| SEQ ID NO. | Sequence |
|---|---|
| 1638 | DTYTKRQLLK |
| 1639 | GTAAMLLASK |
| 1640 | IVPCLSELHK |
| 1641 | SLLEADPFLK |
| 1642 | LLKMEHLLLK |
| 1643 | GVVLATVAR |
| 1644 | CVRTENLAK |
| 1645 | RTCGQGITR |
| 1646 | LVEVGEEYK |
| 1647 | YLAVNFLDR |

In some embodiments, the donor cell source is HLA-A*24:02, and the Cyclin-A₁ targeted T-cell subpopulation is primed and expanded with one or more Cyclin-A₁-derived peptides selected from Table 165 (Seq. ID. Nos. 1648-1657). In some embodiments, the donor cell source is HLA-A*24:02, and the Cyclin-A₁ targeted T-cell subpopulation is primed and expanded with Cyclin-A₁-derived peptides selected from Table 165 (Seq. ID. Nos. 1648-1657). In some embodiments, the donor cell source is HLA-A*24:02, and the Cyclin-A₁ targeted T-cell subpopulation is primed and expanded with Cyclin-A₁-derived peptides comprising the peptides of Table 165 (Seq. ID. Nos. 1648-1657). In some embodiments, the donor cell source is HLA-A*24:02, and the Cyclin-A₁ targeted T-cell subpopulation is primed and expanded with Cyclin-A₁-derived peptides comprising the peptides of Table 165 (Seq. ID. Nos. 1648-1657), and at least one additional set of peptides based on the donor cell source HLA-A profile, wherein the at least one additional set of peptides are selected from the peptides of Tables 161-164 and 166-167. In some embodiments, the Cyclin-A₁-derived peptides also include one or more sets of HLA-B and HLA-DR restricted peptides selected from Tables 168-180 (Seq. ID Nos. 1678-1806).

TABLE 165
Cyclin A₁ HLA-A*24:02 Epitope Peptides

| SEQ ID NO. | Sequence |
|---|---|
| 1648 | EYLSWEGPGL |
| 1649 | EYAEEIYQYL |
| 1650 | PFLKYLPSLI |
| 1651 | IYQYLREAEI |
| 1652 | IYPPEVDEF |
| 1653 | KYVAELSLL |
| 1654 | VYEVDTGTL |
| 1655 | HYMKKQPDI |
| 1656 | EYKLRAETL |
| 1657 | CYSGSENAF |

In some embodiments, the donor cell source is HLA-A*26, and the Cyclin-A₁ targeted T-cell subpopulation is primed and expanded with one or more Cyclin-A₁-derived peptides selected from Table 166 (Seq. ID. Nos. 1658-1667). In some embodiments, the donor cell source is HLA-A*26, and the Cyclin-A₁ targeted T-cell subpopulation is primed and expanded with Cyclin-A₁-derived peptides selected from Table 166 (Seq. ID. Nos. 1658-1667). In some embodiments, the donor cell source is HLA-A*26, and the Cyclin-A₁ targeted T-cell subpopulation is primed and expanded with Cyclin-A₁-derived peptides comprising the peptides of Table 166 (Seq. ID. Nos. 1658-1667). In some embodiments, the donor cell source is HLA-A*26, and the Cyclin-A₁ targeted T-cell subpopulation is primed and expanded with Cyclin-A₁-derived peptides comprising the peptides of Table 166 (Seq. ID. Nos. 1658-1667) and at least one additional set of peptides based on the donor cell source HLA-A profile, wherein the at least one additional set of peptides are selected from the peptides of Tables 161-165 and 167. In some embodiments, the Cyclin-A₁-derived peptides also include one or more sets of HLA-B and HLA-DR restricted peptides selected from Tables 168-180 (Seq. ID Nos. 1678-1806).

TABLE 166
Cyclin A₁ HLA-A*26 Epitope Peptides

| SEQ ID NO. | Sequence |
|---|---|
| 1658 | DVYEVDTGTL |
| 1659 | ETLYLAVNFL |
| 1660 | EIYPPEVDEF |
| 1661 | DPFLKYLPSL |
| 1662 | EIVPCLSEL |
| 1663 | ETGFPAIMY |
| 1664 | ETLAAFTGY |
| 1665 | EYAEEIYQY |
| 1666 | DTGTLKSDL |
| 1667 | EVDEFVYIT |

In some embodiments, the donor cell source is HLA-A*68:01, and the Cyclin-A₁ targeted T-cell subpopulation is primed and expanded with one or more Cyclin-A₁-derived peptides selected from Table 167 (Seq. ID. Nos. 1668-1677). In some embodiments, the donor cell source is HLA-A*68:01, and the Cyclin-A₁ targeted T-cell subpopulation is primed and expanded with Cyclin-A₁-derived peptides selected from Table 167 (Seq. ID. Nos. 1668-1677). In some embodiments, the donor cell source is HLA-A*68:01, and the Cyclin-A₁ targeted T-cell subpopulation is primed and expanded with Cyclin-A₁-derived peptides comprising the peptides of Table 167 (Seq. ID. Nos. 1668-1677). In some embodiments, the donor cell source is HLA-A*68:01, and the Cyclin-A₁ targeted T-cell subpopulation is primed and expanded with Cyclin-A₁-derived peptides comprising the peptides of Table 167 (Seq. ID. Nos. 1668-1677), and at least one additional set of peptides based on the donor cell source HLA-A profile, wherein the at least one additional set of peptides are selected from the peptides of Tables 161-166. In some embodiments, the Cyclin-A₁-derived peptides also include one or more sets of HLA-B and HLA-DR restricted peptides selected from Tables 168-180 (Seq. ID Nos. 1678-1806).

TABLE 167
Cyclin A₁ HLA-A*68:01 Epitope Peptides

| SEQ ID NO. | Sequence |
|---|---|
| 1668 | DVYEVDTGTLK |
| 1669 | DIPHRPQQAIR |
| 1670 | DTYTKRQLLK |
| 1671 | TAAMLLASK |
| 1672 | ITDDTYTKR |
| 1673 | GVVLATVAR |
| 1674 | EVGEEYKLR |
| 1675 | RTCGQGITR |
| 1676 | DACQILTRA |
| 1677 | KAYLDIPHR |

In some embodiments, the donor cell source is HLA-B*07:02, and the Cyclin-A₁ targeted T-cell subpopulation is primed and expanded with one or more Cyclin-A₁-derived peptides selected from Table 168 (Seq. ID. Nos. 1678-1687). In some embodiments, the donor cell source is HLA-B*07:02, and the Cyclin-A₁ targeted T-cell subpopulation is primed and expanded with Cyclin-A₁-derived peptides selected from Table 168 (Seq. ID. Nos. 1678-1687). In some embodiments, the donor cell source is HLA-B*07:02, and the Cyclin-A₁ targeted T-cell subpopulation is primed and expanded with Cyclin-A₁-derived peptides comprising the peptides of Table 168 (Seq. ID. Nos. 1678-1687). In some embodiments, the donor cell source is HLA-B*07:02, and the Cyclin-A₁ targeted T-cell subpopulation is primed and expanded with Cyclin-A₁-derived peptides comprising the peptides of Table 168 (Seq. ID. Nos. 1678-1687), and at least one additional set of peptides based on the donor cell source HLA-B profile, wherein the at least one additional set of peptides are selected from the peptides of Tables 169-174. In some embodiments, the Cyclin-A₁-derived peptides also include one or more sets of HLA-A and HLA-DR restricted peptides selected from Tables 161-167 and 175-180 (Seq. ID Nos. 1606-1677 and 1747-1806).

TABLE 168
Cyclin A₁ HLA-B*07:02 Epitope Peptides

| SEQ ID NO. | Sequence |
|---|---|
| 1678 | DPPQRTVLGL |
| 1679 | SPMLVDSSLL |
| 1680 | DPFLKYLPSL |
| 1681 | NPKSGVVLAT |
| 1682 | LPSLIAAAAF |
| 1683 | PPQRTVLGL |
| 1684 | FPPAGKKAL |
| 1685 | GPGLPDFVF |
| 1686 | IPHRPQQAI |
| 1687 | VPTTNQFLL |

In some embodiments, the donor cell source is HLA-B*08, and the Cyclin-A₁ targeted T-cell subpopulation is primed and expanded with one or more Cyclin-A₁-derived peptides selected from Table 169 (Seq. ID. Nos. 1688-1697). In some embodiments, the donor cell source is HLA-B*08, and the Cyclin-A₁ targeted T-cell subpopulation is primed and expanded with Cyclin-A₁-derived peptides selected from Table 169 (Seq. ID. Nos. 1688-1697). In some embodiments, the donor cell source is HLA-B*08, and the Cyclin-A₁ targeted T-cell subpopulation is primed and expanded with Cyclin-A₁-derived peptides comprising the peptides of Table 169 (Seq. ID. Nos. 1688-1697). In some embodiments, the donor cell source is HLA-B*08, and the Cyclin-A₁ targeted T-cell subpopulation is primed and expanded with Cyclin-A₁-derived peptides comprising the peptides of Table 169 (Seq. ID. Nos. 1688-1697) and at least one additional set of peptides based on the donor cell source HLA-B profile, wherein the at least one additional set of peptides are selected from the peptides of Tables 168 and 170-174. In some embodiments, the Cyclin-A₁-derived peptides also include one or more sets of HLA-A and HLA-DR restricted peptides selected from Tables 161-167 and 175-180 (Seq. ID Nos. 1606-1677 and 1747-1806).

TABLE 169
Cyclin A₁ HLA-B*08 Epitope Peptides

| SEQ ID NO. | Sequence |
|---|---|
| 1688 | EYKLRAETL |
| 1689 | LLKVLAFDL |
| 1690 | TLKSDLHFL |
| 1691 | LLKMEHLLL |
| 1692 | HLLLKVLAF |
| 1693 | VLRGKLQL |
| 1694 | FLKYLPSL |
| 1695 | NPKSGVVL |
| 1696 | PPAGKKAL |
| 1697 | LAKYVAEL |

In some embodiments, the donor cell source is HLA-B*15:01, and the Cyclin-A₁ targeted T-cell subpopulation is primed and expanded with one or more Cyclin-A₁-derived peptides selected from Table 170 (Seq. ID. Nos. 1698-1707). In some embodiments, the donor cell source is HLA-B*15:01, and the Cyclin-A₁ targeted T-cell subpopulation is primed and expanded with Cyclin-A₁-derived peptides selected from Table 170 (Seq. ID. Nos. 1698-1707). In some embodiments, the donor cell source is HLA-B*15:01, and the Cyclin-A₁ targeted T-cell subpopulation is primed and expanded with Cyclin-A₁-derived peptides comprising the peptides of Table 170 (Seq. ID. Nos. 1698-1707). In some embodiments, the donor cell source is HLA-B*15:01, and the Cyclin-A₁ targeted T-cell subpopulation is primed and expanded with Cyclin-A₁-derived peptides comprising the peptides of Table 170 (Seq. ID. Nos. 1698-1707) and at least one additional set of peptides based on the donor cell source HLA-B profile, wherein the at least one additional set of peptides are selected from the peptides of Tables 168-169 and 171-174. In some embodiments, the Cyclin-A₁-derived peptides also include one or more sets of HLA-A and HLA-DR restricted peptides selected from Tables 161-167 and 175-180 (Seq. ID Nos. 1606-1677 and 1747-1806).

TABLE 170
Cyclin A₁ HLA-B*15:01 (B62) Epitope Peptides

| SEQ ID NO. | Sequence |
|---|---|
| 1698 | CVRTENLAKY |
| 1699 | GQGITRIRCY |
| 1700 | LLASKYEEIY |
| 1701 | LLEADPFLKY |
| 1702 | WLVEVGEEY |
| 1703 | PQQAIREKY |
| 1704 | CLSELHKAY |
| 1705 | GLLTANGQY |
| 1706 | HLLLKVLAF |
| 1707 | VQEPPKQGF |

In some embodiments, the donor cell source is HLA-B*18, and the Cyclin-A₁ targeted T-cell subpopulation is primed and expanded with one or more Cyclin-A₁-derived peptides selected from Table 171 (Seq. ID. Nos. 1708-1717). In some embodiments, the donor cell source is HLA-B*18, and the Cyclin-A₁ targeted T-cell subpopulation is primed and expanded with Cyclin-A₁-derived peptides selected from Table 171 (Seq. ID. Nos. 1708-1717). In some embodiments, the donor cell source is HLA-B*18, and the Cyclin-A₁ targeted T-cell subpopulation is primed and expanded with Cyclin-A₁-derived peptides comprising the peptides of Table 171 (Seq. ID. Nos. 1708-1717). In some embodiments, the donor cell source is HLA-B*18, and the Cyclin-A₁ targeted T-cell subpopulation is primed and expanded with Cyclin-A₁-derived peptides comprising the peptides of Table 171 (Seq. ID. Nos. 1708-1717) and at least one additional set of peptides based on the donor cell source HLA-B profile, wherein the at least one additional set of peptides are selected from the peptides of Tables 168-170 and 172-174. In some embodiments, the Cyclin-A₁-derived peptides also include one or more sets of HLA-A and HLA-DR restricted peptides selected from Tables 161-167 and 175-180 (Seq. ID Nos. 1606-1677 and 1747-1806).

TABLE 171
Cyclin A₁ HLA-B*18 Epitope Peptides

| SEQ ID NO. | Sequence |
|---|---|
| 1708 | MEHLLLKVL |
| 1709 | WEGPGLPDF |
| 1710 | LEADPFLKY |
| 1711 | REKYKASKY |
| 1712 | PEVDEFVY |
| 1713 | YEVDTGTL |
| 1714 | QEPPKQGF |
| 1715 | TEYAEEIY |
| 1716 | AEEIYQYL |
| 1717 | TEGMRTIL |

In some embodiments, the donor cell source is HLA-B*27:05, and the Cyclin-A₁ targeted T-cell subpopulation is primed and expanded with one or more Cyclin-A₁-derived peptides selected from Table 172 (Seq. ID. Nos. 1718-1727). In some embodiments, the donor cell source is HLA-B*27:05, and the Cyclin-A₁ targeted T-cell subpopulation is primed and expanded with Cyclin-A₁-derived peptides selected from Table 172 (Seq. ID. Nos. 1718-1727). In some embodiments, the donor cell source is HLA-B*27:05, and the Cyclin-A₁ targeted T-cell subpopulation is primed and expanded with Cyclin-A₁-derived peptides comprising the peptides of Table 172 (Seq. ID. Nos. 1718-1727). In some embodiments, the donor cell source is HLA-B*27:05, and the Cyclin-A₁ targeted T-cell subpopulation is primed and expanded with Cyclin-A₁-derived peptides comprising the peptides of Table 172 (Seq. ID. Nos. 1718-1727) and at least one additional set of peptides based on the donor cell source HLA-B profile, wherein the at least one additional set of peptides are selected from the peptides of Tables 168-171 and 173-174. In some embodiments, the Cyclin-A₁-derived peptides also include one or more sets of HLA-A and HLA-DR restricted peptides selected from Tables 161-167 and 175-180 (Seq. ID Nos. 1606-1677 and 1747-1806).

TABLE 172
Cyclin A₁ HLA-B*27:05 Epitope Peptides

| SEQ ID NO. | Sequence |
|---|---|
| 1718 | RRTCGQGITR |
| 1719 | DRFLSCMSVL |
| 1720 | HRPQQAIREK |
| 1721 | HRPKAHYMKK |
| 1722 | IREKYKASKY |
| 1723 | KRQLLKMEHL |
| 1724 | LRRQGVCVR |
| 1725 | IREKYKASK |
| 1726 | KRQLLKMEH |
| 1727 | LREAEIRHR |

In some embodiments, the donor cell source is HLA-B*35:01, and the Cyclin-A₁ targeted T-cell subpopulation is primed and expanded with one or more Cyclin-A₁-derived peptides selected from Table 173 (Seq. ID. Nos. 1728-1736). In some embodiments, the donor cell source is HLA-B*35:01, and the Cyclin-A₁ targeted T-cell subpopulation is primed and expanded with Cyclin-A₁-derived peptides selected from Table 173 (Seq. ID. Nos. 1728-1736). In some embodiments, the donor cell source is HLA-B*35:01, and the Cyclin-A₁ targeted T-cell subpopulation is primed and expanded with Cyclin-A₁-derived peptides comprising the peptides of Table 173 (Seq. ID. Nos. 1728-1736). In some embodiments, the donor cell source is HLA-B*35:01, and the Cyclin-A₁ targeted T-cell subpopulation is primed and expanded with Cyclin-A₁-derived peptides comprising the peptides of Table 173 (Seq. ID. Nos. 1728-1736) and at least one additional set of peptides based on the donor cell source HLA-B profile, wherein the at least one additional set of peptides are selected from the peptides of Tables 168-172 and 174. In some embodiments, the Cyclin-A₁-derived peptides also include one or more sets of HLA-A and HLA-DR restricted peptides selected from Tables 161-167 and 175-180 (Seq. ID Nos. 1606-1677 and 1747-1806).

TABLE 173
Cyclin A₁ HLA-B*35:01 Epitope Peptides

| SEQ ID NO. | Sequence |
|---|---|
| 1728 | PPQRTVLGLL |
| 1729 | EPPKQGFDIY |
| 1730 | SPMLVDSSLL |
| 1731 | DPFLKYLPSL |
| 1732 | LPSLIAAAAF |
| 1733 | FPPAGKKAL |
| 1734 | DPPQRTVLGL |
| 1735 | RPQQAIREKY |
| 1736 | FPAIMYPGSF |

In some embodiments, the donor cell source is HLA-B*58:02, and the Cyclin-A₁ targeted T-cell subpopulation is primed and expanded with one or more Cyclin-A₁-derived peptides selected from Table 174 (Seq. ID. Nos. 1737-1746). In some embodiments, the donor cell source is HLA-B*58:02, and the Cyclin-A₁ targeted T-cell subpopulation is primed and expanded with Cyclin-A₁-derived peptides selected from Table 174 (Seq. ID. Nos. 1737-1746). In some embodiments, the donor cell source is HLA-B*58:02, and the Cyclin-A₁ targeted T-cell subpopulation is primed and expanded with Cyclin-A₁-derived peptides comprising the peptides of Table 174 (Seq. ID. Nos. 1737-1746). In some embodiments, the donor cell source is HLA-B*58:02, and the Cyclin-A₁ targeted T-cell subpopulation is primed and expanded with Cyclin-A₁-derived peptides comprising the peptides of Table 174 (Seq. ID. Nos. 1737-1746) and at least one additional set of peptides based on the donor cell source HLA-B profile, wherein the at least one additional set of peptides are selected from the peptides of Tables 168-173. In some embodiments, the Cyclin-A₁-derived peptides also include one or more sets of HLA-A and HLA-DR restricted peptides selected from Tables 161-167 and 175-180 (Seq. ID Nos. 1606-1677 and 1747-1806).

TABLE 174
Cyclin A₁ HLA-B*58:02 Epitope Peptides

| SEQ ID NO. | Sequence |
|---|---|
| 1737 | KSDLHFLLDF |
| 1738 | KASKYLCVSL |
| 1739 | RTCGQGITRI |
| 1740 | LAVNFLDRFL |
| 1741 | YSLSEIVPCL |
| 1742 | CSNPKSGVVL |
| 1743 | SSLLSQSEDI |
| 1744 | VSLMEPPAVL |
| 1745 | LSLLEADPF |
| 1746 | PSLIAAAAF |

In some embodiments, the donor cell source is HLA-DRB1*0101, and the Cyclin-A₁ targeted T-cell subpopulation is primed and expanded with one or more Cyclin-A₁-derived peptides selected from Table 175 (Seq. ID. Nos. 1747-1756). In some embodiments, the donor cell source is HLA-DRB1*0101, and the Cyclin-A₁ targeted T-cell subpopulation is primed and expanded with Cyclin-A₁-derived peptides selected from Table 175 (Seq. ID. Nos. 1747-1756). In some embodiments, the donor cell source is HLA-DRB1*0101, and the Cyclin-A₁ targeted T-cell subpopulation is primed and expanded with Cyclin-A₁-derived peptides comprising the peptides of Table 175 (Seq. ID. Nos. 1747-1756). In some embodiments, the donor cell source is HLA-DRB1*0101, and the Cyclin-A₁ targeted T-cell subpopulation is primed and expanded with Cyclin-A₁-derived peptides comprising the peptides of Table 175 (Seq. ID. Nos. 1747-1756) and at least one additional set of peptides based on the donor cell source HLA-DR profile, wherein the at least one additional set of peptides are selected from the peptides of Tables 176-180. In some embodiments, the Cyclin-A₁-derived peptides also include one or more sets of HLA-A and HLA-B restricted peptides selected from Tables 161-174 (Seq. ID Nos. 1606-1746).

TABLE 175
Cyclin A₁ HLA-DRB1*0101 Epitope Peptides

| SEQ ID NO. | Sequence |
|---|---|
| 1747 | VAELSLLEADPFLKY |
| 1748 | EHLLLKVLAFDLTVP |
| 1749 | LKYLPSLIAAAAFCL |
| 1750 | LLDFNTVSPMLVDSS |
| 1751 | SEDISSLGTDVINVT |
| 1752 | DPFLKYLPSLIAAAA |
| 1753 | NGQYRRTCGQGITRI |
| 1754 | RGKLQLVGTAAMLLA |
| 1755 | ASKYEEIYPPEVDEF |
| 1756 | YLCVSLMEPPAVLLL |

In some embodiments, the donor cell source is HLA-DRB1*0301, and the Cyclin-A₁ targeted T-cell subpopulation is primed and expanded with one or more Cyclin-A₁-derived peptides selected from Table 176 (Seq. ID. Nos. 1757-1766). In some embodiments, the donor cell source is HLA-DRB1*0301, and the Cyclin-A₁ targeted T-cell subpopulation is primed and expanded with Cyclin-A₁-derived peptides selected from Table 176 (Seq. ID. Nos. 1757-1766). In some embodiments, the donor cell source is HLA-DRB1*0301, and the Cyclin-A₁ targeted T-cell subpopulation is primed and expanded with Cyclin-A₁-derived peptides comprising the peptides of Table 176 (Seq. ID. Nos. 1757-1766). In some embodiments, the donor cell source is HLA-DRB1*0301, and the Cyclin-A₁ targeted T-cell subpopulation is primed and expanded with Cyclin-A₁-derived peptides comprising the peptides of Table 176 (Seq. ID. Nos. 1757-1766) and at least one additional set of peptides based on the donor cell source HLA-DR profile, wherein the at least one additional set of peptides are selected from the peptides of Tables 175 and 177-180. In some embodiments, the Cyclin-A₁-derived peptides also include one or more sets of HLA-A and HLA-B restricted peptides selected from Tables 161-174 (Seq. ID Nos. 1606-1746).

TABLE 176
Cyclin A₁ HLA-DRB1*0301 (DR17) Epitope Peptides

| SEQ ID NO. | Sequence |
|---|---|
| 1757 | TGTLKSDLITFLLDFN |
| 1758 | LSLLEADPFLKYLPS |
| 1759 | DCGVQEPPKQGFDIY |
| 1760 | DLHFLLDFNTVSPML |
| 1761 | EAEIRHRPKAHYMKK |
| 1762 | VINVTEYAEEIYQYL |
| 1763 | QPDITEGMRTILVDW |
| 1764 | DWLVEVGEEYKLRAE |
| 1765 | VPCLSELHKAYLDIP |
| 1766 | GSFIGGWGEEYLSWE |

In some embodiments, the donor cell source is HLA-DRB1*0401, and the Cyclin-A₁ targeted T-cell subpopulation is primed and expanded with one or more Cyclin-A₁-derived peptides selected from Table 177 (Seq. ID. Nos. 1767-1776). In some embodiments, the donor cell source is HLA-DRB1*0401, and the Cyclin-A₁ targeted T-cell subpopulation is primed and expanded with Cyclin-A₁-derived peptides selected from Table 177 (Seq. ID. Nos. 1767-1776). In some embodiments, the donor cell source is HLA-DRB1*0401, and the Cyclin-A₁ targeted T-cell subpopulation is primed and expanded with Cyclin-A₁-derived peptides comprising the peptides of Table 177 (Seq. ID. Nos. 1767-1776). In some embodiments, the donor cell source is HLA-DRB1*0401, and the Cyclin-A₁ targeted T-cell subpopulation is primed and expanded with Cyclin-A₁-derived peptides comprising the peptides of Table 177 (Seq. ID. Nos. 1767-1776) and at least one additional set of peptides based on the donor cell source HLA-DR profile, wherein the at least one additional set of peptides are selected from the peptides of Tables 175-176 and 178-180. In some embodiments, the Cyclin-A₁-derived peptides also include one or more sets of HLA-A and HLA-B restricted peptides selected from Tables 161-174 (Seq. ID Nos. 1606-1746).

TABLE 177
Cyclin A₁ HLA-DRB1*0401 (DR4Dw4) Epitope Peptides

| SEQ ID NO. | Sequence |
|---|---|
| 1767 | DLHFLLDFNTVSPML |
| 1768 | EFVYITDDTYTKRQL |
| 1769 | HKAYLDIPHRPQQAI |
| 1770 | FEDVYEVDTGTLKSD |
| 1771 | TGTLKSDLRFLLDFN |
| 1772 | VSPMLVDSSLLSQSE |
| 1773 | SEDISSLGTDVINVT |
| 1774 | YLAVNFLDRFLSCMS |
| 1775 | VNFLDRFLSCMSVLR |
| 1776 | RGKLQLVGTAAMLLA |

In some embodiments, the donor cell source is HLA-DRB1*0701, and the Cyclin-A₁ targeted T-cell subpopulation is primed and expanded with one or more Cyclin-A₁-derived peptides selected from Table 178 (Seq. ID. Nos. 1777-1786). In some embodiments, the donor cell source is HLA-DRB1*0701, and the Cyclin-A₁ targeted T-cell subpopulation is primed and expanded with Cyclin-A₁-derived peptides selected from Table 178 (Seq. ID. Nos. 1777-1786). In some embodiments, the donor cell source is HLA-DRB1*0701, and the Cyclin-A₁ targeted T-cell subpopulation is primed and expanded with Cyclin-A₁-derived peptides comprising the peptides of Table 178 (Seq. ID. Nos. 1777-1786). In some embodiments, the donor cell source is HLA-DRB1*0701, and the Cyclin-A₁ targeted T-cell subpopulation is primed and expanded with Cyclin-A₁-derived peptides comprising the peptides of Table 178 (Seq. ID. Nos. 1777-1786) and at least one additional set of peptides based on the donor cell source HLA-DR profile, wherein the at least one additional set of peptides are selected from the peptides of Tables 175-177 and 179-180. In some embodiments, the Cyclin-A₁-derived peptides also include one or more sets of HLA-A and HLA-B restricted peptides selected from Tables 161-174 (Seq. ID Nos. 1606-1746).

TABLE 178
Cyclin A₁ HLA-DRB1*0701 Epitope Peptides

| SEQ ID NO. | Sequence |
|---|---|
| 1777 | VDEFVYITDDTYTKR |
| 1778 | NGQYRRTCGQGITRI |
| 1779 | ENAFPPAGKKALPDC |
| 1780 | FEDVYEVDTGTLKSD |
| 1781 | FLLDFNTVSPMLVDS |
| 1782 | SEDISSLGTDVINVT |
| 1783 | EEIYQYLREAEIRHR |
| 1784 | RGKLQLVGTAAMLLA |
| 1785 | KLQLVGTAAMLLASK |
| 1786 | ASKYEEIYPPEVDEF |

In some embodiments, the donor cell source is HLA-DRB1*1101, and the Cyclin-A₁ targeted T-cell subpopulation is primed and expanded with one or more Cyclin-A₁-derived peptides selected from Table 179 (Seq. ID. Nos. 1787-1796). In some embodiments, the donor cell source is HLA-DRB1*1101, and the Cyclin-A₁ targeted T-cell subpopulation is primed and expanded with Cyclin-A₁-derived peptides selected from Table 179 (Seq. ID. Nos. 1787-1796). In some embodiments, the donor cell source is HLA-DRB1*1101, and the Cyclin-A₁ targeted T-cell subpopulation is primed and expanded with Cyclin-A₁-derived peptides comprising the peptides of Table 179 (Seq. ID. Nos. 1787-1796). In some embodiments, the donor cell source is HLA-DRB1*1101, and the Cyclin-A₁ targeted T-cell subpopulation is primed and expanded with Cyclin-A₁-derived peptides comprising the peptides of Table 179 (Seq. ID. Nos. 1787-1796) and at least one additional set of peptides based on the donor cell source HLA-DR profile, wherein the at least one additional set of peptides are selected from the peptides of Tables 175-178 and 180. In some embodiments, the Cyclin-A₁-derived peptides also include one or more sets of HLA-A and HLA-B restricted peptides selected from Tables 161-174 (Seq. ID Nos. 1606-1746).

TABLE 179
Cyclin A₁ HLA-DRB1*1101 Epitope Peptides

| SEQ ID NO. | Sequence |
|---|---|
| 1787 | YLAVNFLDRFLSCMS |
| 1788 | LANYTVNKHFWPETL |
| 1789 | HKAYLDIPHRPQQAI |
| 1790 | ETGFPAIMYPGSFIG |
| 1791 | LVEVGEEYKLRAETL |
| 1792 | ASKYLCVSLMEPPAV |
| 1793 | KRQLLKMEHLLLKVL |
| 1794 | LKMEHLLLKVLAFDL |
| 1795 | NQFLLQYLRRQGVCV |
| 1796 | CVRTENLAKYVAELS |

In some embodiments, the donor cell source is HLA-DRB1*1501, and the Cyclin-A₁ targeted T-cell subpopulation is primed and expanded with one or more Cyclin-A₁-derived peptides selected from Table 180 (Seq. ID. Nos. 1797-1806). In some embodiments, the donor cell source is HLA-DRB1*1501, and the Cyclin-A₁ targeted T-cell subpopulation is primed and expanded with Cyclin-A₁-derived peptides selected from Table 180 (Seq. ID. Nos. 1797-1806). In some embodiments, the donor cell source is HLA-DRB1*1501, and the Cyclin-A₁ targeted T-cell subpopulation is primed and expanded with Cyclin-A₁-derived peptides comprising the peptides of Table 180 (Seq. ID. Nos. 1797-1806). In some embodiments, the donor cell source is HLA-DRB1*1501, and the Cyclin-A₁ targeted T-cell subpopulation is primed and expanded with Cyclin-A₁-derived peptides comprising the peptides of Table 180 (Seq. ID. Nos. 1797-1806) and at least one additional set of peptides based on the donor cell source HLA-DR profile, wherein the at least one additional set of peptides are selected from the peptides of Tables 175-179. In some embodiments, the Cyclin-A₁-derived peptides also include one or more sets of HLA-A and HLA-B restricted peptides selected from Tables 161-174 (Seq. ID Nos. 1606-1746).

TABLE 180
Cyclin A₁ HLA-DRB1*1501 (DR2b) Epitope Peptides

| SEQ ID NO. | Sequence |
|---|---|
| 1797 | YLAVNFLDRFLSCMS |
| 1798 | LANYTVNKHFWPETL |
| 1799 | HKAYLDIPHRPQQAI |
| 1800 | ETGFPAIMYPGSFIG |
| 1801 | LVEVGEEYKLRAETL |
| 1802 | ASKYLCVSLMEPPAV |
| 1803 | KRQLLKMEHLLLKVL |
| 1804 | LKMEHLLLKVLAFDL |
| 1805 | NQFLLQYLRRQGVCV |
| 1806 | CVRTENLAKYVAELS |

Neutrophil Elastase Antigenic Peptides

In some embodiments, the MUSTANG composition includes neutrophil elastase specific T-cells. neutrophil elastase specific T-cells can be generated as described below using one or more antigenic peptides to neutrophil elastase. In some embodiments, the neutrophil elastase specific T-cells are generated using one or more antigenic peptides to neutrophil elastase, or a modified or heteroclitic peptide derived from a neutrophil elastase peptide. In some embodiments, neutrophil elastase specific T-cells are generated using a neutrophil elastase antigen library comprising a pool of peptides (for example 15mers) containing amino acid overlap (for example 11 amino acids of overlap) between each sequence formed by scanning the protein amino acid sequence SEQ. ID. No. 1807 (UniProt KB-P08246) for neutrophil elastase:

MTLGRRLACLFLACVLPALLLGGTALASEIVGGRRARPHAWPFMVSLQL
RGGHFCGATLIAPNFVMSAAHCVANVNVRAVRVVLGAHNLSRREPTRQV
FAVQRIFENGYDPVNLLNDIVILQLNGSATINANVQVAQLPAQGRRLGN
GVQCLAMGWGLLGRNRGIASVLQELNVTVVTSLCRRSNVCTLVRGRQAG
VCFGDSGSPLVCNGLIHGIASFVRGGCASGLYPDAFAPVAQFVNWIDSI
IQRSEDNPCPHPRDPDPASRTH.

In some embodiments, the neutrophil elastase specific T-cells are generated using one or more antigenic peptides to neutrophil elastase, or a modified or heteroclitic peptide derived from a neutrophil elastase peptide. In some embodiments, the neutrophil elastase specific T-cells are generated with peptides that recognize class I MHC molecules. In some embodiments, the neutrophil elastase specific T-cells are generated with peptides that recognize class II MHC molecules. In some embodiments, the neutrophil elastase specific T-cells are generated with peptides that recognize both class I and class II MHC molecules.

In some embodiments, the neutrophil elastase peptides used to prime and expand a T-cell subpopulation includes specifically selected HLA-restricted peptides generated by determining the HLA profile of the donor source, and including peptides derived from neutrophil elastase that best match the donor's HLA. In some embodiments, the neutrophil elastase peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MEW ligands and peptide motifs. Immunogenetics (1999) 50: 213. https://doi.org/10.1007/s002510050595.

As provided herein, the HLA profile of a donor cell source can be determined, and T-cell subpopulations targeting neutrophil elastase derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 181-187, the HLA-B peptides are selected from the peptides of Tables 188-194, and the HLA-DR peptides are selected from the peptides of Tables 195-200. For example, if the donor cell source has an HLA profile that is HLA-A*01/*02:01; HLA-B*15:01/*18; and HLA-DRB1*0101/*0301, then the neutrophil elastase peptides used to prime and expand the neutrophil elastase specific T-cell subpopulation are restricted to the specific HLA profile, and may include the peptides identified in Table 181 (Seq. ID. Nos. 1808-1817) for HLA-A*01; Table 182 (Seq. ID. Nos. 1818-1827) for HLA-A*02:01; Table 190 (Seq. ID. Nos. 1989-1907) for HLA-B*15:01; Table 191 (Seq. ID. Nos. 1908-1917) for HLA-B*18; Table 195 (Seq. ID. Nos. 1948-1957) for HLA-DRB1*0101; and Table 196 (Seq. ID. Nos. 1958-1967) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the neutrophil elastase targeted T-cell subpopulation is primed and expanded with one or more neutrophil elastase-derived peptides selected from Table 181 (Seq. ID. Nos. 1808-1817). In some embodiments, the donor cell source is HLA-A*01, and the neutrophil elastase targeted T-cell subpopulation is primed and expanded with neutrophil elastase-derived peptides selected from Table 181 (Seq. ID. Nos. 1808-1817). In some embodiments, the donor cell source is HLA-A*01, and the neutrophil elastase targeted T-cell subpopulation is primed and expanded with neutrophil elastase-derived peptides comprising the peptides of from Table 181 (Seq. ID. Nos. 1808-1817). In some embodiments, the donor cell source is HLA-A*01, and the neutrophil elastase targeted T-cell subpopulation is primed and expanded with neutrophil elastase-derived peptides comprising the peptides of from Table 181 (Seq. ID. Nos. 1808-1817) and at least one additional set of peptides based on the donor cell source HLA-A profile, wherein the at least one additional set of peptides are selected from the peptides of Tables 182-187. In some embodiments, the neutrophil elastase-derived peptides also include one or more sets of HLA-B and HLA-DR restricted peptides selected from Tables 188-200 (Seq. ID Nos. 1878-2007).

TABLE 181
Neutrophil Elastase HLA-A*01 Epitope Peptides

| SEQ ID NO. | Sequence |
|---|---|
| 1808 | FVRGGCASGLY |
| 1809 | FAVQRIFENGY |
| 1810 | AVQRIFENGY |
| 1811 | GYDPVNLLND |
| 1812 | VRGGCASGLY |
| 1813 | ASEIVGGRRA |
| 1814 | FGDSGSPLVC |
| 1815 | VQRIFENGY |
| 1816 | RGGCASGLY |
| 1817 | LNDIVILQL |

In some embodiments, the donor cell source is HLA-A*02:01, and the neutrophil elastase targeted T-cell subpopulation is primed and expanded with one or more neutrophil elastase-derived peptides selected from Table 182 (Seq. ID. Nos. 1818-1827). In some embodiments, the donor cell source is HLA-A*02:01, and the neutrophil elastase targeted T-cell subpopulation is primed and expanded with neutrophil elastase-derived peptides selected from Table 182 (Seq. ID. Nos. 1818-1827). In some embodiments, the donor cell source is HLA-A*02:01, and the neutrophil elastase targeted T-cell subpopulation is primed and expanded with neutrophil elastase-derived peptides comprising the peptides of Table 182 (Seq. ID. Nos. 1818-1827). In some embodiments, the donor cell source is HLA-A*02:01, and the neutrophil elastase targeted T-cell subpopulation is primed and expanded with neutrophil elastase-derived peptides comprising the peptides of Table 182 (Seq. ID. Nos. 1818-1827) and at least one additional set of peptides based on the donor cell source HLA-A profile, wherein the at least one additional set of peptides are selected from the peptides of Tables 181, and 183-187. In some embodiments, the neutrophil elastase-derived peptides also include one or more sets of HLA-B and HLA-DR restricted peptides selected from Tables 188-200 (Seq. ID Nos. 1878-2007).

TABLE 182
Neutrophil Elastase HLA-A*02:01 Epitope Peptides

| SEQ ID NO. | Sequence |
|---|---|
| 1818 | LLNDIVILQL |
| 1819 | RLACLFLACV |
| 1820 | GLYPDAFAPV |
| 1821 | FLACVLPAL |
| 1822 | NLLNDIVIL |
| 1823 | VLQELNVTV |
| 1824 | ALLLGGTAL |
| 1825 | GIASVLQEL |
| 1826 | TLGRRLACL |
| 1827 | LLLGGTALA |

In some embodiments, the donor cell source is HLA-A*03, and the neutrophil elastase targeted T-cell subpopulation is primed and expanded with one or more neutrophil elastase-derived peptides selected from Table 183 (Seq. ID. Nos. 1828-1837). In some embodiments, the donor cell source is HLA-A*03, and the neutrophil elastase targeted T-cell subpopulation is primed and expanded with neutrophil elastase-derived peptides selected from Table 183 (Seq. ID. Nos. 1828-1837). In some embodiments, the donor cell source is HLA-A*03, and the neutrophil elastase targeted T-cell subpopulation is primed and expanded with neutrophil elastase-derived peptides comprising the peptides of Table 183 (Seq. ID. Nos. 1828-1837). In some embodiments, the donor cell source is HLA-A*03, and the neutrophil elastase targeted T-cell subpopulation is primed and expanded with neutrophil elastase-derived peptides comprising the peptides of Table 183 (Seq. ID. Nos. 1828-1837) and at least one additional set of peptides based on the donor cell source HLA-A profile, wherein the at least one additional set of peptides are selected from the peptides of Tables 181-182 and 184-187. In some embodiments, the neutrophil elastase-derived peptides also include one or more sets of HLA-B and HLA-DR restricted peptides selected from Tables 188-200 (Seq. ID Nos. 1878-2007).

TABLE 183
Neutrophil Elastase HLA-A*03 Epitope Peptides

| SEQ ID NO. | Sequence |
|---|---|
| 1828 | VVLGAHNLSR |
| 1829 | LVRGRQAGVC |
| 1830 | AVQRIFENGY |
| 1831 | NVRAVRVVL |
| 1832 | GLIHGIASF |
| 1833 | ALLLGGTAL |
| 1834 | AVRVVLGAH |
| 1835 | NLSRREPTR |
| 1836 | SLQLRGGHF |
| 1837 | FVRGGCASG |

In some embodiments, the donor cell source is HLA-A*11:01, and the neutrophil elastase targeted T-cell subpopulation is primed and expanded with one or more neutrophil elastase-derived peptides selected from Table 184 (Seq. ID. Nos. 1838-1847). In some embodiments, the donor cell source is HLA-A*11:01, and the neutrophil elastase targeted T-cell subpopulation is primed and expanded with neutrophil elastase-derived peptides selected from Table 184 (Seq. ID. Nos. 1838-1847). In some embodiments, the donor cell source is HLA-A*11:01, and the neutrophil elastase targeted T-cell subpopulation is primed and expanded with neutrophil elastase-derived peptides comprising the peptides of Table 184 (Seq. ID. Nos. 1838-1847). In some embodiments, the donor cell source is HLA-A*11:01, and the neutrophil elastase targeted T-cell subpopulation is primed and expanded with neutrophil elastase-derived peptides comprising the peptides of Table 184 (Seq. ID. Nos. 1838-1847), and at least one additional set of peptides based on the donor cell source HLA-A profile, wherein the at least one additional set of peptides are selected from the peptides of Tables 181-183 and 185-187. In some embodiments, the neutrophil elastase-derived peptides also include one or more sets of HLA-B and HLA-DR restricted peptides selected from Tables 188-200 (Seq. ID Nos. 1878-2007).

TABLE 184
Neutrophil Elastase HLA-A*11:01 Epitope Peptides

| SEQ ID NO. | Sequence |
|---|---|
| 1838 | VVLGAHNLSR |
| 1839 | PTRQVFAVQR |
| 1840 | NVTVVTSLCR |
| 1841 | ALASEIVGGR |
| 1842 | QVAQLPAQGR |
| 1843 | RSNVCTLVR |
| 1844 | VTVVTSLCR |
| 1845 | ASEIVGGRR |
| 1846 | AMGWGLLGR |
| 1847 | NVCTLVRGR |

In some embodiments, the donor cell source is HLA-A*24:02, and the neutrophil elastase targeted T-cell subpopulation is primed and expanded with one or more neutrophil elastase-derived peptides selected from Table 185 (Seq. ID. Nos. 1848-1857). In some embodiments, the donor cell source is HLA-A*24:02, and the neutrophil elastase targeted T-cell subpopulation is primed and expanded with neutrophil elastase-derived peptides selected from Table 185 (Seq. ID. Nos. 1848-1857). In some embodiments, the donor cell source is HLA-A*24:02, and the neutrophil elastase targeted T-cell subpopulation is primed and expanded with neutrophil elastase-derived peptides comprising the peptides of Table 185 (Seq. ID. Nos. 1848-1857). In some embodiments, the donor cell source is HLA-A*24:02, and the neutrophil elastase targeted T-cell subpopulation is primed and expanded with neutrophil elastase-derived peptides comprising the peptides of Table 185 (Seq. ID. Nos. 1848-1857), and at least one additional set of peptides based on the donor cell source HLA-A profile, wherein the at least one additional set of peptides are selected from the peptides of Tables 181-184 and 186-187. In some embodiments, the neutrophil elastase-derived peptides also include one or more sets of HLA-B and HLA-DR restricted peptides selected from Tables 188-200 (Seq. ID Nos. 1878-2007).

TABLE 185
Neutrophil Elastase HLA-A*24:02 Epitope Peptides

| SEQ ID NO. | Sequence |
|---|---|
| 1848 | LFLACVLPAL |
| 1849 | VSLQLRGGHF |
| 1850 | VNLLNDIVIL |
| 1851 | QCLAMGWGLL |
| 1852 | GSPLVCNGLI |
| 1853 | CFGDSGSPL |
| 1854 | QFVNWIDSI |
| 1855 | NGYDPVNLL |
| 1856 | GYDPVNLLN |
| 1857 | LYPDAFAPV |

In some embodiments, the donor cell source is HLA-A*26, and the neutrophil elastase targeted T-cell subpopulation is primed and expanded with one or more neutrophil elastase-derived peptides selected from Table 186 (Seq. ID. Nos. 1858-1867). In some embodiments, the donor cell source is HLA-A*26, and the neutrophil elastase targeted T-cell subpopulation is primed and expanded with neutrophil elastase-derived peptides selected from Table 186 (Seq. ID. Nos. 1858-1867). In some embodiments, the donor cell source is HLA-A*26, and the neutrophil elastase targeted T-cell subpopulation is primed and expanded with neutrophil elastase-derived peptides comprising the peptides of Table 186 (Seq. ID. Nos. 1858-1867). In some embodiments, the donor cell source is HLA-A*26, and the neutrophil elastase targeted T-cell subpopulation is primed and expanded with neutrophil elastase-derived peptides comprising the peptides of Table 186 (Seq. ID. Nos. 1858-1867) and at least one additional set of peptides based on the donor cell source HLA-A profile, wherein the at least one additional set of peptides are selected from the peptides of Tables 181-185 and 187. In some embodiments, the neutrophil elastase-derived peptides also include one or more sets of HLA-B and HLA-DR restricted peptides selected from Tables 188-200 (Seq. ID Nos. 1878-2007).

TABLE 186
Neutrophil Elastase HLA-A*26 Epitope Peptides

| SEQ ID NO. | Sequence |
|---|---|
| 1858 | ELNVTVVTSL |
| 1859 | ENGYDPVNLL |
| 1860 | MTLGRRLACL |
| 1861 | AVQRIFENGY |
| 1862 | DIVILQLNGS |
| 1863 | FVRGGCASGL |
| 1864 | DAFAPVAQF |
| 1865 | RVVLGAHNL |
| 1866 | QVFAVQRIF |
| 1867 | EIVGGRRAR |

In some embodiments, the donor cell source is HLA-A*68:01, and the neutrophil elastase targeted T-cell subpopulation is primed and expanded with one or more neutrophil elastase-derived peptides selected from Table 187 (Seq. ID. Nos. 1868-1877). In some embodiments, the donor cell source is HLA-A*68:01, and the neutrophil elastase targeted T-cell subpopulation is primed and expanded with neutrophil elastase-derived peptides selected from Table 187 (Seq. ID. Nos. 1868-1877). In some embodiments, the donor cell source is HLA-A*68:01, and the neutrophil elastase targeted T-cell subpopulation is primed and expanded with neutrophil elastase-derived peptides comprising the peptides of Table 187 (Seq. ID. Nos. 1868-1877). In some embodiments, the donor cell source is HLA-A*68:01, and the neutrophil elastase targeted T-cell subpopulation is primed and expanded with neutrophil elastase-derived peptides comprising the peptides of Table 187 (Seq. ID. Nos. 1868-1877), and at least one additional set of peptides based on the donor cell source HLA-A profile, wherein the at least one additional set of peptides are selected from the peptides of Tables 181-186. In some embodiments, the neutrophil elastase-derived peptides also include one or more sets of HLA-B and HLA-DR restricted peptides selected from Tables 188-200 (Seq. ID Nos. 1878-2007).

TABLE 187
Neutrophil Elastase HLA-A*68:01 Epitope Peptides

| SEQ ID NO. | Sequence |
|---|---|
| 1868 | CVANVNVRAVR |
| 1869 | FVNWIDSIIQR |
| 1870 | QVAQLPAQGRR |
| 1871 | PTRQVFAVQR |
| 1872 | VANVNVRAVR |
| 1873 | TVVTSLCRR |
| 1874 | LASEIVGGR |
| 1875 | NVCTLVRGR |
| 1876 | NWIDSIIQR |
| 1877 | EIVGGRRAR |

In some embodiments, the donor cell source is HLA-B*07:02, and the neutrophil elastase targeted T-cell subpopulation is primed and expanded with one or more neutrophil elastase-derived peptides selected from Table 188 (Seq. ID. Nos. 1878-1887). In some embodiments, the donor cell source is HLA-B*07:02, and the neutrophil elastase targeted T-cell subpopulation is primed and expanded with neutrophil elastase-derived peptides selected from Table 188 (Seq. ID. Nos. 1878-1887). In some embodiments, the donor cell source is HLA-B*07:02, and the neutrophil elastase targeted T-cell subpopulation is primed and expanded with neutrophil elastase-derived peptides comprising the peptides of Table 188 (Seq. ID. Nos. 1878-1887). In some embodiments, the donor cell source is HLA-B*07:02, and the neutrophil elastase targeted T-cell subpopulation is primed and expanded with neutrophil elastase-derived peptides comprising the peptides of Table 188 (Seq. ID. Nos. 1878-1887), and at least one additional set of peptides based on the donor cell source HLA-B profile, wherein the at least one additional set of peptides are selected from the peptides of Tables 189-194. In some embodiments, the neutrophil elastase-derived peptides also include one or more sets of HLA-A and HLA-DR restricted peptides selected from Tables 181-187 and 195-200 (Seq. ID Nos. 1808-1877 and 1948-2007).

TABLE 188
Neutrophil Elastase HLA-B*07:02 Epitope Peptides

| SEQ ID NO. | Sequence |
|---|---|
| 1878 | APVAQFVNWI |
| 1879 | CPHPRDPDPA |
| 1880 | LPALLLGGTA |
| 1881 | WPFMVSLQL |
| 1882 | YPDAFAPVA |
| 1883 | APNFVMSAA |
| 1884 | EPTRQVFAV |
| 1885 | RPHAWPFMV |
| 1886 | ACVLPALLL |
| 1887 | NVRAVRVVL |

In some embodiments, the donor cell source is HLA-B*08, and the neutrophil elastase targeted T-cell subpopulation is primed and expanded with one or more neutrophil elastase-derived peptides selected from Table 189 (Seq. ID. Nos. 1888-1897). In some embodiments, the donor cell source is HLA-B*08, and the neutrophil elastase targeted T-cell subpopulation is primed and expanded with neutrophil elastase-derived peptides selected from Table 189 (Seq. ID. Nos. 1888-1897). In some embodiments, the donor cell source is HLA-B*08, and the neutrophil elastase targeted T-cell subpopulation is primed and expanded with neutrophil elastase-derived peptides comprising the peptides of Table 189 (Seq. ID. Nos. 1888-1897). In some embodiments, the donor cell source is HLA-B*08, and the neutrophil elastase targeted T-cell subpopulation is primed and expanded with neutrophil elastase-derived peptides comprising the peptides of Table 189 (Seq. ID. Nos. 1888-1897) and at least one additional set of peptides based on the donor cell source HLA-B profile, wherein the at least one additional set of peptides are selected from the peptides of Tables 188 and 190-194. In some embodiments, the neutrophil elastase-derived peptides also include one or more sets of HLA-A and HLA-DR restricted peptides selected from Tables 181-187 and 195-200 (Seq. ID Nos. 1808-1877 and 1948-2007).

TABLE 189
Neutrophil Elastase HLA-B*08 Epitope Peptides

| SEQ ID NO. | Sequence |
|---|---|
| 1888 | TLGRRLACL |
| 1889 | SLQLRGGHF |
| 1890 | GLLGRNRGI |
| 1891 | GRRLACLFL |
| 1892 | FLACVLPAL |
| 1893 | ALLLGGTAL |
| 1894 | NLLNDIVIL |
| 1895 | VRAVRVVL |
| 1896 | SPLVCNGL |
| 1897 | CLFLACVL |

In some embodiments, the donor cell source is HLA-B*15:01, and the neutrophil elastase targeted T-cell subpopulation is primed and expanded with one or more neutrophil elastase-derived peptides selected from Table 190 (Seq. ID. Nos. 1898-1907). In some embodiments, the donor cell source is HLA-B*15:01, and the neutrophil elastase targeted T-cell subpopulation is primed and expanded with neutrophil elastase-derived peptides selected from Table 190 (Seq. ID. Nos. 1898-1907). In some embodiments, the donor cell source is HLA-B*15:01, and the neutrophil elastase targeted T-cell subpopulation is primed and expanded with neutrophil elastase-derived peptides comprising the peptides of Table 190 (Seq. ID. Nos. 1898-1907). In some embodiments, the donor cell source is HLA-B*15:01, and the neutrophil elastase targeted T-cell subpopulation is primed and expanded with neutrophil elastase-derived peptides comprising the peptides of Table 190 (Seq. ID. Nos. 1898-1907) and at least one additional set of peptides based on the donor cell source HLA-B profile, wherein the at least one additional set of peptides are selected from the peptides of Tables 188-189 and 191-194. In some embodiments, the neutrophil elastase-derived peptides also include one or more sets of HLA-A and HLA-DR restricted peptides selected from Tables 181-187 and 195-200 (Seq. ID Nos. 1808-1877 and 1948-2007).

TABLE 190
Neutrophil Elastase HLA-B*15:01 (B62)
Epitope Peptides

| SEQ ID NO. | Sequence |
|---|---|
| 1898 | RQVFAVQRIF |
| 1899 | TLGRRLACLF |
| 1900 | AVQRIFENGY |
| 1901 | ALLLGGTALA |
| 1902 | ILQLNGSATI |
| 1903 | RLGNGVQCLA |
| 1904 | VQRIFENGY |
| 1905 | GLIHGIASF |
| 1906 | SLQLRGGHF |
| 1907 | QVFAVQRIF |

In some embodiments, the donor cell source is HLA-B*18, and the neutrophil elastase targeted T-cell subpopulation is primed and expanded with one or more neutrophil elastase-derived peptides selected from Table 191 (Seq. ID. Nos. 1908-1917). In some embodiments, the donor cell source is HLA-B*18, and the neutrophil elastase targeted T-cell subpopulation is primed and expanded with neutrophil elastase-derived peptides selected from Table 191 (Seq. ID. Nos. 1908-1917). In some embodiments, the donor cell source is HLA-B*18, and the neutrophil elastase targeted T-cell subpopulation is primed and expanded with neutrophil elastase-derived peptides comprising the peptides of Table 191 (Seq. ID. Nos. 1908-1917). In some embodiments, the donor cell source is HLA-B*18, and the neutrophil elastase targeted T-cell subpopulation is primed and expanded with neutrophil elastase-derived peptides comprising the peptides of Table 191 (Seq. ID. Nos. 1908-1917) and at least one additional set of peptides based on the donor cell source HLA-B profile, wherein the at least one additional set of peptides are selected from the peptides of Tables 188-190 and 192-194. In some embodiments, the neutrophil elastase-derived peptides also include one or more sets of HLA-A and HLA-DR restricted peptides selected from Tables 181-187 and 195-200 (Seq. ID Nos. 1808-1877 and 1948-2007).

TABLE 191
Neutrophil Elastase HLA-B*18 Epitope Peptides

| SEQ ID NO. | Sequence |
|---|---|
| 1908 | DAFAPVAQF |
| 1909 | LGRRLACLF |
| 1910 | NVRAVRVVL |
| 1911 | ASGLYPDAF |
| 1912 | ACLFLACVL |
| 1913 | GATLIAPNF |
| 1914 | REPTRQVF |
| 1915 | QELNVTVV |
| 1916 | ANVQVAQL |
| 1917 | ACVLPALL |

In some embodiments, the donor cell source is HLA-B*27:05, and the neutrophil elastase targeted T-cell subpopulation is primed and expanded with one or more neutrophil elastase-derived peptides selected from Table 192 (Seq. ID. Nos. 1918-1927). In some embodiments, the donor cell source is HLA-B*27:05, and the neutrophil elastase targeted T-cell subpopulation is primed and expanded with neutrophil elastase-derived peptides selected from Table 192 (Seq. ID. Nos. 1918-1927). In some embodiments, the donor cell source is HLA-B*27:05, and the neutrophil elastase targeted T-cell subpopulation is primed and expanded with neutrophil elastase-derived peptides comprising the peptides of Table 192 (Seq. ID. Nos. 1918-1927). In some embodiments, the donor cell source is HLA-B*27:05, and the neutrophil elastase targeted T-cell subpopulation is primed and expanded with neutrophil elastase-derived peptides comprising the peptides of Table 192 (Seq. ID. Nos. 1918-1927) and at least one additional set of peptides based on the donor cell source HLA-B profile, wherein the at least one additional set of peptides are selected from the peptides of Tables 188-191 and 193-194. In some embodiments, the neutrophil elastase-derived peptides also include one or more sets of HLA-A and HLA-DR restricted peptides selected from Tables 181-187 and 195-200 (Seq. ID Nos. 1808-1877 and 1948-2007).

TABLE 192
Neutrophil Elastase HLA-B*27:05 Epitope Peptides

| SEQ ID NO. | Sequence |
|---|---|
| 1918 | RRLGNGVQCL |
| 1919 | RRARPHAWPF |
| 1920 | GRNRGIASVL |
| 1921 | RRSNVCTLVR |
| 1922 | VRVVLGAHNL |
| 1923 | RREPTRQVF |
| 1924 | GRRLACLFL |
| 1925 | VRGGCASGL |
| 1926 | TRQVFAVQR |
| 1927 | PRDPDPASR |

In some embodiments, the donor cell source is HLA-B*35:01, and the neutrophil elastase targeted T-cell subpopulation is primed and expanded with one or more neutrophil elastase-derived peptides selected from Table 193 (Seq. ID. Nos. 1928-1937). In some embodiments, the donor cell source is HLA-B*35:01, and the neutrophil elastase targeted T-cell subpopulation is primed and expanded with neutrophil elastase-derived peptides selected from Table 193 (Seq. ID. Nos. 1928-1937). In some embodiments, the donor cell source is HLA-B*35:01, and the neutrophil elastase targeted T-cell subpopulation is primed and expanded with neutrophil elastase-derived peptides comprising the peptides of Table 193 (Seq. ID. Nos. 1928-1937). In some embodiments, the donor cell source is HLA-B*35:01, and the neutrophil elastase targeted T-cell subpopulation is primed and expanded with neutrophil elastase-derived peptides comprising the peptides of Table 193 (Seq. ID. Nos. 1928-1937) and at least one additional set of peptides based on the donor cell source HLA-B profile, wherein the at least one additional set of peptides are selected from the peptides of Tables 188-192 and 194. In some embodiments, the neutrophil elastase-derived peptides also include one or more sets of HLA-A and HLA-DR restricted peptides selected from Tables 181-187 and 195-200 (Seq. ID Nos. 1808-1877 and 1948-2007).

TABLE 193
Neutrophil Elastase HLA-B*35:01 Epitope Peptides

| SEQ ID NO. | Sequence |
|---|---|
| 1928 | APVAQFVNWI |
| 1929 | LFLACVLPAL |
| 1930 | VNVRAVRVVL |
| 1931 | YPDAFAPVAQ |
| 1932 | WPFMVSLQL |
| 1933 | DPVNLLNDI |
| 1934 | SPLVCNGLI |
| 1935 | ACLFLACVL |
| 1936 | NLLNDIVIL |
| 1937 | RNRGIASVL |

In some embodiments, the donor cell source is HLA-B*58:02, and the neutrophil elastase targeted T-cell subpopulation is primed and expanded with one or more neutrophil elastase-derived peptides selected from Table 194 (Seq. ID. Nos. 1938-1947). In some embodiments, the donor cell source is HLA-B*58:02, and the neutrophil elastase targeted T-cell subpopulation is primed and expanded with neutrophil elastase-derived peptides selected from Table 194 (Seq. ID. Nos. 1938-1947). In some embodiments, the donor cell source is HLA-B*58:02, and the neutrophil elastase targeted T-cell subpopulation is primed and expanded with neutrophil elastase-derived peptides comprising the peptides of Table 194 (Seq. ID. Nos. 1938-1947). In some embodiments, the donor cell source is HLA-B*58:02, and the neutrophil elastase targeted T-cell subpopulation is primed and expanded with neutrophil elastase-derived peptides comprising the peptides of Table 194 (Seq. ID. Nos. 1938-1947) and at least one additional set of peptides based on the donor cell source HLA-B profile, wherein the at least one additional set of peptides are selected from the peptides of Tables 188-193. In some embodiments, the neutrophil elastase-derived peptides also include one or more sets of HLA-A and HLA-DR restricted peptides selected from Tables 181-187 and 195-200 (Seq. ID Nos. 1808-1877 and 1948-2007).

TABLE 194
Neutrophil Elastase HLA-B*58:02 Epitope Peptides

| SEQ ID NO. | Sequence |
|---|---|
| 1938 | RARPHAWPFM |
| 1939 | VSLQLRGGHF |
| 1940 | GSPLVCNGLI |
| 1941 | LACVLPALLL |
| 1942 | RVVLGAHNL |
| 1943 | QVFAVQRIF |
| 1944 | GATLIAPNF |
| 1945 | ASGLYPDAF |
| 1946 | GATLIAPNF |
| 1947 | ASGLYPDAF |

In some embodiments, the donor cell source is HLA-DRB1*0101, and the neutrophil elastase targeted T-cell subpopulation is primed and expanded with one or more neutrophil elastase-derived peptides selected from Table 195 (Seq. ID. Nos. 1948-1957). In some embodiments, the donor cell source is HLA-DRB1*0101, and the neutrophil elastase targeted T-cell subpopulation is primed and expanded with neutrophil elastase-derived peptides selected from Table 195 (Seq. ID. Nos. 1948-1957). In some embodiments, the donor cell source is HLA-DRB1*0101, and the neutrophil elastase targeted T-cell subpopulation is primed and expanded with neutrophil elastase-derived peptides comprising the peptides of Table 195 (Seq. ID. Nos. 1948-1957). In some embodiments, the donor cell source is HLA-DRB1*0101, and the neutrophil elastase targeted T-cell subpopulation is primed and expanded with neutrophil elastase-derived peptides comprising the peptides of Table 195 (Seq. ID. Nos. 1948-1957) and at least one additional set of peptides based on the donor cell source HLA-DR profile, wherein the at least one additional set of peptides are selected from the peptides of Tables 196-200. In some embodiments, the neutrophil elastase-derived peptides also include one or more sets of HLA-A and HLA-B restricted peptides selected from Tables 181-194 (Seq. ID Nos. 1808-1947).

TABLE 195
Neutrophil Elastase HLA-DRB1*0101
Epitope Peptides

| SEQ ID NO. | Sequence |
|---|---|
| 1948 | PHAWPFMVSLQLRGG |
| 1949 | LNDIVILQLNGSATI |
| 1950 | CLFLACVLPALLLGG |
| 1951 | APNFVMSAAHCVANV |
| 1952 | ASFVRGGCASGLYPD |
| 1953 | VLPALLLGGTALASE |
| 1954 | ALASEIVGGRRARPH |
| 1955 | RAVRVVLGAHNLSRR |
| 1956 | YDPVNLLNDIVILQL |
| 1957 | NVQVAQLPAQGRRLG |

In some embodiments, the donor cell source is HLA-DRB1*0301, and the neutrophil elastase targeted T-cell subpopulation is primed and expanded with one or more neutrophil elastase-derived peptides selected from Table 196 (Seq. ID. Nos. 1958-1967). In some embodiments, the donor cell source is HLA-DRB1*0301, and the neutrophil elastase targeted T-cell subpopulation is primed and expanded with neutrophil elastase-derived peptides selected from Table 196 (Seq. ID. Nos. 1958-1967). In some embodiments, the donor cell source is HLA-DRB1*0301, and the neutrophil elastase targeted T-cell subpopulation is primed and expanded with neutrophil elastase-derived peptides comprising the peptides of Table 196 (Seq. ID. Nos. 1958-1967). In some embodiments, the donor cell source is HLA-DRB1*0301, and the neutrophil elastase targeted T-cell subpopulation is primed and expanded with neutrophil elastase-derived peptides comprising the peptides of Table 196 (Seq. ID. Nos. 1958-1967) and at least one additional set of peptides based on the donor cell source HLA-DR profile, wherein the at least one additional set of peptides are selected from the peptides of Tables 195 and 197-200. In some embodiments, the neutrophil elastase-derived peptides also include one or more sets of HLA-A and HLA-B restricted peptides selected from Tables 181-194 (Seq. ID Nos. 1808-1947).

TABLE 196
Neutrophil Elastase HLA-DRB1*0301 (DR17)
Epitope Peptides

| SEQ ID NO. | Sequence |
|---|---|
| 1958 | VFAVQRIFENGYDPV |
| 1959 | PVNLLNDIVILQLNG |
| 1960 | ASGLYPDAFAPVAQF |
| 1961 | CLFLACVLPALLLGG |
| 1962 | IFENGYDPVNLLNDI |
| 1963 | IASVLQELNVTVVTS |
| 1964 | ACLFLACVLPALLLG |
| 1965 | VRVVLGAHNLSRREP |
| 1966 | VNLLNDIVILQLNGS |
| 1967 | NDIVILQLNGSATIN |

In some embodiments, the donor cell source is HLA-DRB1*0401, and the neutrophil elastase targeted T-cell subpopulation is primed and expanded with one or more neutrophil elastase-derived peptides selected from Table 197 (Seq. ID. Nos. 1968-1977). In some embodiments, the donor cell source is HLA-DRB1*0401, and the neutrophil elastase targeted T-cell subpopulation is primed and expanded with neutrophil elastase-derived peptides selected from Table 197 (Seq. ID. Nos. 1968-1977). In some embodiments, the donor cell source is HLA-DRB1*0401, and the neutrophil elastase targeted T-cell subpopulation is primed and expanded with neutrophil elastase-derived peptides comprising the peptides of Table 197 (Seq. ID. Nos. 1968-1977). In some embodiments, the donor cell source is HLA-DRB1*0401, and the neutrophil elastase targeted T-cell subpopulation is primed and expanded with neutrophil elastase-derived peptides comprising the peptides of Table 197 (Seq. ID. Nos. 1968-1977) and at least one additional set of peptides based on the donor cell source HLA-DR profile, wherein the at least one additional set of peptides are selected from the peptides of Tables 195-196 and 198-200. In some embodiments, the neutrophil elastase-derived peptides also include one or more sets of HLA-A and HLA-B restricted peptides selected from Tables 181-194 (Seq. ID Nos. 1808-1947).

TABLE 197
Neutrophil Elastase HLA-DRB1*0401 (DR4Dw4)
Epitope Peptides

| SEQ ID NO. | Sequence |
|---|---|
| 1968 | PDAFAPVAQFVNWID |
| 1969 | PNFVMSAAHCVANVN |
| 1970 | NRGIASVLQELNVTV |
| 1971 | IASVLQELNVTVVTS |
| 1972 | VTVVTSLCRRSNVCT |
| 1973 | RSNVCTLVRGRQAGV |
| 1974 | AQFVNWIDSIIQRSE |
| 1975 | PHAWPFMVSLQLRGG |
| 1976 | AMGWGLLGRNRGIAS |
| 1977 | GVCFGDSGSPLVCNG |

In some embodiments, the donor cell source is HLA-DRB1*0701, and the neutrophil elastase targeted T-cell subpopulation is primed and expanded with one or more neutrophil elastase-derived peptides selected from Table 198 (Seq. ID. Nos. 1978-1987). In some embodiments, the donor cell source is HLA-DRB1*0701, and the neutrophil elastase targeted T-cell subpopulation is primed and expanded with neutrophil elastase-derived peptides selected from Table 198 (Seq. ID. Nos. 1978-1987). In some embodiments, the donor cell source is HLA-DRB1*0701, and the neutrophil elastase targeted T-cell subpopulation is primed and expanded with neutrophil elastase-derived peptides comprising the peptides of Table 198 (Seq. ID. Nos. 1978-1987). In some embodiments, the donor cell source is HLA-DRB1*0701, and the neutrophil elastase targeted T-cell subpopulation is primed and expanded with neutrophil elastase-derived peptides comprising the peptides of Table 198 (Seq. ID. Nos. 1978-1987) and at least one additional set of peptides based on the donor cell source HLA-DR profile, wherein the at least one additional set of peptides are selected from the peptides of Tables 195-197 and 199-200. In some embodiments, the neutrophil elastase-derived peptides also include one or more sets of HLA-A and HLA-B restricted peptides selected from Tables 181-194 (Seq. ID Nos. 1808-1947).

TABLE 198
Neutrophil Elastase HLA-DRB1*0701
Epitope Peptides

| SEQ ID NO. | Sequence |
|---|---|
| 1978 | GVCFGDSGSPLVCNG |
| 1979 | PHAWPFMVSLQLRGG |
| 1980 | VFAVQRIFENGYDPV |
| 1981 | ILQLNGSATINANVQ |
| 1982 | APNFVMSAAHCVANV |
| 1983 | IVILQLNGSATINAN |
| 1984 | LQELNVTVVTSLCRR |
| 1985 | VAQFVNWIDSIIQRS |
| 1986 | YDPVNLLNDIVILQL |
| 1987 | VNLLNDIVILQLNGS |

In some embodiments, the donor cell source is HLA-DRB1*1101, and the neutrophil elastase targeted T-cell subpopulation is primed and expanded with one or more neutrophil elastase-derived peptides selected from Table 199 (Seq. ID. Nos. 1988-1997). In some embodiments, the donor cell source is HLA-DRB1*1101, and the neutrophil elastase targeted T-cell subpopulation is primed and expanded with neutrophil elastase-derived peptides selected from Table 199 (Seq. ID. Nos. 1988-1997). In some embodiments, the donor cell source is HLA-DRB1*1101, and the neutrophil elastase targeted T-cell subpopulation is primed and expanded with neutrophil elastase-derived peptides comprising the peptides of Table 199 (Seq. ID. Nos. 1988-1997). In some embodiments, the donor cell source is HLA-DRB1*1101, and the neutrophil elastase targeted T-cell subpopulation is primed and expanded with neutrophil elastase-derived peptides comprising the peptides of Table 199 (Seq. ID. Nos. 1988-1997) and at least one additional set of peptides based on the donor cell source HLA-DR profile, wherein the at least one additional set of peptides are selected from the peptides of Tables 195-198 and 200. In some embodiments, the neutrophil elastase-derived peptides also include one or more sets of HLA-A and HLA-B restricted peptides selected from Tables 181-194 (Seq. ID Nos. 1808-1947).

TABLE 199
Neutrophil Elastase HLA-DRB1*1101
Epitope Peptides

| SEQ ID NO. | Sequence |
|---|---|
| 1988 | AMGWGLLGRNRGIAS |
| 1989 | RSNVCTLVRGRQAGV |
| 1990 | VTVVTSLCRRSNVCT |
| 1991 | ASEIVGGRRARPHAW |
| 1992 | VAQLPAQGRRLGNGV |
| 1993 | PNFVMSAAHCVANVN |
| 1994 | AVRVVLGAHNLSRRE |
| 1995 | VLGAHNLSRREPTRQ |
| 1996 | TRQVFAVQRIFENGY |
| 1997 | GWGLLGRNRGIASVL |

In some embodiments, the donor cell source is HLA-DRB1*1501, and the neutrophil elastase targeted T-cell subpopulation is primed and expanded with one or more neutrophil elastase-derived peptides selected from Table 200 (Seq. ID. Nos. 1998-2007). In some embodiments, the donor cell source is HLA-DRB1*1501, and the neutrophil elastase targeted T-cell subpopulation is primed and expanded with neutrophil elastase-derived peptides selected from Table 200 (Seq. ID. Nos. 1998-2007). In some embodiments, the donor cell source is HLA-DRB1*1501, and the neutrophil elastase targeted T-cell subpopulation is primed and expanded with neutrophil elastase-derived peptides comprising the peptides of Table 200 (Seq. ID. Nos. 1998-2007). In some embodiments, the donor cell source is HLA-DRB1*1501, and the neutrophil elastase targeted T-cell subpopulation is primed and expanded with neutrophil elastase-derived peptides comprising the peptides of Table 200 (Seq. ID. Nos. 1998-2007) and at least one additional set of peptides based on the donor cell source HLA-DR profile, wherein the at least one additional set of peptides are selected from the peptides of Tables 195-199. In some embodiments, the neutrophil elastase-derived peptides also include one or more sets of HLA-A and HLA-B restricted peptides selected from Tables 181-194 (Seq. ID Nos. 1808-1947).

TABLE 200
Neutrophil Elastase HLA-DRB1*1501 (DR2b)
Epitope Peptides

| SEQ ID NO. | Sequence |
|---|---|
| 1998 | PALLLGGTALASEIV |
| 1999 | VRVVLGAHNLSRREP |
| 2000 | RIFENGYDPVNLLND |
| 2001 | ILQLNGSATINANVQ |
| 2002 | VQCLAMGWGLLGRNR |
| 2003 | WGLLGRNRGIASVLQ |
| 2004 | IASVLQELNVTVVTS |
| 2005 | ELNVTVVTSLCRRSN |
| 2006 | VTSLCRRSNVCTLVR |
| 2007 | NGLIHGIASFVRGGC |

Epstein-Barr Virus (EBV) Strain B95-8 MP 1 Antigenic Peptides

In some embodiments, the MUSTANG composition includes Epstein-Barr Virus (EBV) Strain B95-8 LMP1 specific T-cells. LMP1 specific T-cells can be generated as described below using one or more antigenic peptides to LMP1. In some embodiments, the LMP1 specific T-cells are generated using one or more antigenic peptides to LMP1, or a modified or heteroclitic peptide derived from a LMP1 peptide. In some embodiments, LMP1 specific T-cells are generated using a LMP1 antigen library comprising a pool of peptides (for example 15mers) containing amino acid overlap (for example 11 amino acids of overlap) between each sequence formed by scanning the protein amino acid sequence SEQ. ID. No. 2008 (UniProt KB-P03230) for EBV Strain B95-8 LMP1:

MEHDLERGPPGPRRPPRGPPLSSSLGLALLLLLLALLFWLYIVMSDWTG
GALLVLYSFALMLIIIILIIFIFRRDLLCPLGALCILLLMITLLLIALW
NLHGQALFLGIVLFIFGCLLVLGIWIYLLEMLWRLGATIWQLLAFFLAF
FLDLILLIIALYLQQNWWTLLVDLLWLLLFLAILIWMYYHGQRHSDEHH
HDDSLPHPQQATDDSGHESDSNSNEGRHHLLVSGAGDGPPLCSQNLGAP
GGGPDNGPQDPDNTDDNGPQDPDNTDDNGPHDPLPQDPDNTDDNGPQDP
DNTDDNGPHDPLPHSPSDSAGNDGGPPQLTEEVENKGGDQGPPLMTDGG
GGHSHDSGHGGGDPHLPTLLLGSSGSGGDDDDPHGPVQLSYYD.

In some embodiments, the LMP1 specific T-cells are generated using one or more antigenic peptides to LMP1, or a modified or heteroclitic peptide derived from a LMP1 peptide. In some embodiments, the LMP1 specific T-cells are generated with peptides that recognize class I MHC molecules. In some embodiments, the LMP1 specific T-cells are generated with peptides that recognize class II MHC molecules. In some embodiments, the LMP1 specific T-cells are generated with peptides that recognize both class I and class II MHC molecules.

In some embodiments, the LMP1 peptides used to prime and expand a T-cell subpopulation includes specifically selected HLA-restricted peptides generated by determining the HLA profile of the donor source, and including peptides derived from LMP1 that best match the donor's HLA. In some embodiments, the LMP1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting LMP1 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 201-207, the HLA-B peptides are selected from the peptides of Tables 208-214, and the HLA-DR peptides are selected from the peptides of Tables 215-220. For example, if the donor cell source has an HLA profile that is HLA-A*01/*02:01; HLA-B*15:01/*18; and HLA-DRB1*0101/*0301, then the LMP1 peptides used to prime and expand the LMP1 specific T-cell subpopulation are restricted to the specific HLA profile, and may include the peptides identified in Table 201 (Seq. ID. Nos. 2009-2013) for HLA-A*01; Table 202 (Seq. ID. Nos. 2014-2018) for HLA-A*02:01; Table 210 (Seq. ID. Nos. 2054-2058) for HLA-B*15:01; Table 211 (Seq. ID. Nos. 2059-2063) for HLA-B*18; Table 215 (Seq. ID. Nos. 2079-2083) for HLA-DRB1*0101; and Table 216 (Seq. ID. Nos. 2084-2088) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the LMP1 targeted T-cell subpopulation is primed and expanded with one or more LMP1-derived peptides selected from Table 201 (Seq. ID. Nos. 2009-2013). In some embodiments, the donor cell source is HLA-A*01, and the LMP1 targeted T-cell subpopulation is primed and expanded with LMP1-derived peptides selected from Table 201 (Seq. ID. Nos. 2009-2013). In some embodiments, the donor cell source is HLA-A*01, and the LMP1 targeted T-cell subpopulation is primed and expanded with LMP1-derived peptides comprising the peptides of from Table 201 (Seq. ID. Nos. 2009-2013). In some embodiments, the donor cell source is HLA-A*01, and the LMP1 targeted T-cell subpopulation is primed and expanded with LMP1-derived peptides comprising the peptides of from Table 201 (Seq. ID. Nos. 2009-2013) and at least one additional set of peptides based on the donor cell source HLA-A profile, wherein the at least one additional set of peptides are selected from the peptides of Tables 202-207. In some embodiments, the LMP1-derived peptides also include one or more sets of HLA-B and HLA-DR restricted peptides selected from Tables 208-220 (Seq. ID Nos. 2044-2108).

TABLE 201
EBV Strain B95-8 LMP1 HLA-A*01 Epitope Peptides

| SEQ ID NO. | Sequence |
|---|---|
| 2009 | LLALLFWLY |
| 2010 | WTGGALLVLY |
| 2011 | LLLLALLFWLY |
| 2012 | MSDWTGGALLV |
| 2013 | DWTGGALLVLY |

In some embodiments, the donor cell source is HLA-A*02:01, and the LMP1 targeted T-cell subpopulation is primed and expanded with one or more LMP1-derived peptides selected from Table 202 (Seq. ID. Nos. 2014-2018). In some embodiments, the donor cell source is HLA-A*02:01, and the LMP1 targeted T-cell subpopulation is primed and expanded with LMP1-derived peptides selected from Table 202 (Seq. ID. Nos. 2014-2018). In some embodiments, the donor cell source is HLA-A*02:01, and the LMP1 targeted T-cell subpopulation is primed and expanded with LMP1-derived peptides comprising the peptides of Table 202 (Seq. ID. Nos. 2014-2018). In some embodiments, the donor cell source is HLA-A*02:01, and the LMP1 targeted T-cell subpopulation is primed and expanded with LMP1-derived peptides comprising the peptides of Table 202 (Seq. ID. Nos. 2014-2018) and at least one additional set of peptides based on the donor cell source HLA-A profile, wherein the at least one additional set of peptides are selected from the peptides of Tables 201, and 203-207. In some embodiments, the LMP1-derived peptides also include one or more sets of HLA-B and HLA-DR restricted peptides selected from Tables 208-220 (Seq. ID Nos. 2044-2108).

TABLE 202
EBV Strain B95-8 LMP1 HLA-A*02:01
Epitope Peptides

| SEQ ID NO. | Sequence |
|---|---|
| 2014 | ALLLLLLAL |
| 2015 | LLLLLLALL |
| 2016 | YLLEMLWRL |
| 2017 | GLALLLLLL |
| 2018 | LLLALLFWL |

In some embodiments, the donor cell source is HLA-A*03, and the LMP1 targeted T-cell subpopulation is primed and expanded with one or more LMP1-derived peptides selected from Table 203 (Seq. ID. Nos. 2019-2023). In some embodiments, the donor cell source is HLA-A*03, and the LMP1 targeted T-cell subpopulation is primed and expanded with LMP1-derived peptides selected from Table 203 (Seq. ID. Nos. 2019-2023). In some embodiments, the donor cell source is HLA-A*03, and the LMP1 targeted T-cell subpopulation is primed and expanded with LMP1-derived peptides comprising the peptides of Table 203 (Seq. ID. 2019-2023). In some embodiments, the donor cell source is HLA-A*03, and the LMP1 targeted T-cell subpopulation is primed and expanded with LMP1-derived peptides comprising the peptides of Table 203 (Seq. ID. Nos. 2019-2023) and at least one additional set of peptides based on the donor cell source HLA-A profile, wherein the at least one additional set of peptides are selected from the peptides of Tables 201-202 and 204-207. In some embodiments, the LMP1-derived peptides also include one or more sets of HLA-B and HLA-DR restricted peptides selected from Tables 208-220 (Seq. ID Nos. 2044-2108).

TABLE 203
EBV Strain B95-8 LMP1 HLA-A*03 Epitope Peptides

| SEQ ID NO. | Sequence |
|---|---|
| 2019 | ALFLGIVLF |
| 2020 | QLLAFFLAF |
| 2021 | LLLLLALLF |
| 2022 | MLWRLGATI |
| 2023 | QLTEEVENK |

In some embodiments, the donor cell source is HLA-A*11:01, and the LMP1 targeted T-cell subpopulation is primed and expanded with one or more LMP1-derived peptides selected from Table 204 (Seq. ID. Nos. 2024-2028). In some embodiments, the donor cell source is HLA-A*11:01, and the LMP1 targeted T-cell subpopulation is primed and expanded with LMP1-derived peptides selected from Table 204 (Seq. ID. Nos. 2024-2028). In some embodiments, the donor cell source is HLA-A*11:01, and the LMP1 targeted T-cell subpopulation is primed and expanded with LMP1-derived peptides comprising the peptides of Table 204 (Seq. ID. Nos. 2024-2028). In some embodiments, the donor cell source is HLA-A*11:01, and the LMP1 targeted T-cell subpopulation is primed and expanded with LMP1-derived peptides comprising the peptides of Table 204 (Seq. ID. Nos. 2024-2028), and at least one additional set of peptides based on the donor cell source HLA-A profile, wherein the at least one additional set of peptides are selected from the peptides of Tables 201-203 and 205-207. In some embodiments, the LMP1-derived peptides also include one or more sets of HLA-B and HLA-DR restricted peptides selected from Tables 208-220 (Seq. ID Nos. 2044-2108).

TABLE 204
EBV Strain B95-8 LMP1 HLA-A*11:01
Epitope Peptides

| SEQ ID NO. | Sequence |
|---|---|
| 2024 | SSLGLALLL |
| 2025 | IILIIFIFR |
| 2026 | SSSLGLALLL |
| 2027 | IIILIIFIFR |
| 2028 | ESDSNSNEGR |

In some embodiments, the donor cell source is HLA-A*24:02, and the LMP1 targeted T-cell subpopulation is primed and expanded with one or more LMP1-derived peptides selected from Table 205 (Seq. ID. Nos. 2029-2033). In some embodiments, the donor cell source is HLA-A*24:02, and the LMP1 targeted T-cell subpopulation is primed and expanded with LMP1-derived peptides selected from Table 205 (Seq. ID. Nos. 2029-2033). In some embodiments, the donor cell source is HLA-A*24:02, and the LMP1 targeted T-cell subpopulation is primed and expanded with LMP1-derived peptides comprising the peptides of Table 205 (Seq. ID. Nos. 2029-2033). In some embodiments, the donor cell source is HLA-A*24:02, and the LMP1 targeted T-cell subpopulation is primed and expanded with LMP1-derived peptides comprising the peptides of Table 205 (Seq. ID. Nos. 2029-2033), and at least one additional set of peptides based on the donor cell source HLA-A profile, wherein the at least one additional set of peptides are selected from the peptides of Tables 201-204 and 206-207. In some embodiments, the LMP1-derived peptides also include one or more sets of HLA-B and HLA-DR restricted peptides selected from Tables 208-220 (Seq. ID Nos. 2044-2108).

TABLE 205
EBV Strain B95-8 LMP1 HLA-A*24:02
Epitope Peptides

| SEQ ID NO. | Sequence |
|---|---|
| 2029 | LYSFALMLI |
| 2030 | FFLDLILLI |
| 2031 | IFIFRRDLL |
| 2032 | IYLLEMLWRL |
| 2033 | LYLQQNWWTL |

In some embodiments, the donor cell source is HLA-A*26, and the LMP1 targeted T-cell subpopulation is primed and expanded with one or more LMP1-derived peptides selected from Table 206 (Seq. ID. Nos. 2034-2038). In some embodiments, the donor cell source is HLA-A*26, and the LMP1 targeted T-cell subpopulation is primed and expanded with LMP1-derived peptides selected from Table 206 (Seq. ID. Nos. 2034-2038). In some embodiments, the donor cell source is HLA-A*26, and the LMP1 targeted T-cell subpopulation is primed and expanded with LMP1-derived peptides comprising the peptides of Table 206 (Seq. ID. Nos. 2034-2038). In some embodiments, the donor cell source is HLA-A*26, and the LMP1 targeted T-cell subpopulation is primed and expanded with LMP1-derived peptides comprising the peptides of Table 206 (Seq. ID. Nos. 2034-2038) and at least one additional set of peptides based on the donor cell source HLA-A profile, wherein the at least one additional set of peptides are selected from the peptides of Tables 201-205 and 207. In some embodiments, the LMP1-derived peptides also include one or more sets of HLA-B and HLA-DR restricted peptides selected from Tables 208-220 (Seq. ID Nos. 2044-2108).

TABLE 206
EBV Strain B95-8 LMP1 HLA-A*26 Epitope Peptides

| SEQ ID NO. | Sequence |
|---|---|
| 2034 | DLILLIIAL |
| 2035 | ATIWQLLAF |
| 2036 | LIIIILIIF |
| 2037 | EVENKGGDQ |
| 2038 | LVDLLWLLLF |

In some embodiments, the donor cell source is HLA-A*68:01, and the LMP1 targeted T-cell subpopulation is primed and expanded with one or more LMP1-derived peptides selected from Table 207 (Seq. ID. Nos. 2039-2043). In some embodiments, the donor cell source is HLA-A*68:01, and the LMP1 targeted T-cell subpopulation is primed and expanded with LMP1-derived peptides selected from Table 207 (Seq. ID. Nos. 2039-2043). In some embodiments, the donor cell source is HLA-A*68:01, and the LMP1 targeted T-cell subpopulation is primed and expanded with LMP1-derived peptides comprising the peptides of Table 207 (Seq. ID. Nos. 2039-2043). In some embodiments, the donor cell source is HLA-A*68:01, and the LMP1 targeted T-cell subpopulation is primed and expanded with LMP1-derived peptides comprising the peptides of Table 207 (Seq. ID. Nos. 2039-2043), and at least one additional set of peptides based on the donor cell source HLA-A profile, wherein the at least one additional set of peptides are selected from the peptides of Tables 201-206. In some embodiments, the LMP1-derived peptides also include one or more sets of HLA-B and HLA-DR restricted peptides selected from Tables 208-220 (Seq. ID Nos. 2044-2108).

TABLE 207
EBV Strain B95-8 LMP1 HLA-A*68:01
Epitope Peptides

| SEQ ID NO. | Sequence |
|---|---|
| 2039 | IIIILIIFIFR |
| 2040 | IILIIFIFR |
| 2041 | IIILIIFIFR |
| 2042 | ILIIFIFRR |
| 2043 | DLERGPPGPR |

In some embodiments, the donor cell source is HLA-B*07:02, and the LMP1 targeted T-cell subpopulation is primed and expanded with one or more LMP1-derived peptides selected from Table 208 (Seq. ID. Nos. 2044-2048). In some embodiments, the donor cell source is HLA-B*07:02, and the LMP1 targeted T-cell subpopulation is primed and expanded with LMP1-derived peptides selected from Table 208 (Seq. ID. Nos. 2044-2048). In some embodiments, the donor cell source is HLA-B*07:02, and the LMP1 targeted T-cell subpopulation is primed and expanded with LMP1-derived peptides comprising the peptides of Table 208 (Seq. ID. Nos. 2044-2048). In some embodiments, the donor cell source is HLA-B*07:02, and the LMP1 targeted T-cell subpopulation is primed and expanded with LMP1-derived peptides comprising the peptides of Table 208 (Seq. ID. Nos. 2044-2048), and at least one additional set of peptides based on the donor cell source HLA-B profile, wherein the at least one additional set of peptides are selected from the peptides of Tables 209-214. In some embodiments, the LMP1-derived peptides also include one or more sets of HLA-A and HLA-DR restricted peptides selected from Tables 201-207 and 215-220 (Seq. ID Nos. 2009-2043 and 2079-2108).

TABLE 208
EBV Strain B95-8 LMP1 HLA-B*07:02
Epitope Peptides

| SEQ ID NO. | Sequence |
|---|---|
| 2044 | DPHLPTLLL |
| 2045 | PPLSSSLGL |
| 2046 | GPPLCSQNL |
| 2047 | GPPLSSSLGL |
| 2048 | CPLGALCILL |

In some embodiments, the donor cell source is HLA-B*08, and the LMP1 targeted T-cell subpopulation is primed and expanded with one or more LMP1-derived peptides selected from Table 209 (Seq. ID. Nos. 2049-2053). In some embodiments, the donor cell source is HLA-B*08, and the LMP1 targeted T-cell subpopulation is primed and expanded with LMP1-derived peptides selected from Table 209 (Seq. ID. Nos. 2049-2053). In some embodiments, the donor cell source is HLA-B*08, and the LMP1 targeted T-cell subpopulation is primed and expanded with LMP1-derived peptides comprising the peptides of Table 209 (Seq. ID. Nos. 2049-2053). In some embodiments, the donor cell source is HLA-B*08, and the LMP1 targeted T-cell subpopulation is primed and expanded with LMP1-derived peptides comprising the peptides of Table 209 (Seq. ID. Nos. 2049-2053) and at least one additional set of peptides based on the donor cell source HLA-B profile, wherein the at least one additional set of peptides are selected from the peptides of Tables 208 and 210-214. In some embodiments, the LMP1-derived peptides also include one or more sets of HLA-A and HLA-DR restricted peptides selected from Tables 201-207 and 215-220 (Seq. ID Nos. 2009-2043 and 2079-2108).

TABLE 209
EBV Strain B95-8 LMP1 HLA-B*08 Epitope Peptides

| SEQ ID NO. | Sequence |
|---|---|
| 2049 | FIFRRDLL |
| 2050 | SNEGRHHLL |
| 2051 | SLGLALLLL |
| 2052 | ILLLMITLL |
| 2053 | DLILLIIAL |

In some embodiments, the donor cell source is HLA-B*15:01, and the LMP1 targeted T-cell subpopulation is primed and expanded with one or more LMP1-derived peptides selected from Table 210 (Seq. ID. Nos. 2054-2058). In some embodiments, the donor cell source is HLA-B*15:01, and the LMP1 targeted T-cell subpopulation is primed and expanded with LMP1-derived peptides selected from Table 210 (Seq. ID. Nos. 2054-2058). In some embodiments, the donor cell source is HLA-B*15:01, and the LMP1 targeted T-cell subpopulation is primed and expanded with LMP1-derived peptides comprising the peptides of Table 210 (Seq. ID. Nos. 2054-2058). In some embodiments, the donor cell source is HLA-B*15:01, and the LMP1 targeted T-cell subpopulation is primed and expanded with LMP1-derived peptides comprising the peptides of Table 210 (Seq. ID. Nos. 2054-2058) and at least one additional set of peptides based on the donor cell source HLA-B profile, wherein the at least one additional set of peptides are selected from the peptides of Tables 208-209 and 211-214. In some embodiments, the LMP1-derived peptides also include one or more sets of HLA-A and HLA-DR restricted peptides selected from Tables 201-207 and 215-220 (Seq. ID Nos. 2009-2043 and 2079-2108).

TABLE 210
EBV Strain B95-8 LMP1 HLA-B*15:01 (B62)
Epitope Peptides

| SEQ ID NO. | Sequence |
|---|---|
| 2054 | ALFLGIVLF |
| 2055 | CLLVLGIWIY |
| 2056 | LLLALLFWLY |
| 2057 | MLIIIILIIF |
| 2058 | DLILLIIALY |

In some embodiments, the donor cell source is HLA-B*18, and the LMP1 targeted T-cell subpopulation is primed and expanded with one or more LMP1-derived peptides selected from Table 211 (Seq. ID. Nos. 2059-2063). In some embodiments, the donor cell source is HLA-B*18, and the LMP1 targeted T-cell subpopulation is primed and expanded with LMP1-derived peptides selected from Table 211 (Seq. ID. Nos. 2059-2063). In some embodiments, the donor cell source is HLA-B*18, and the LMP1 targeted T-cell subpopulation is primed and expanded with LMP1-derived peptides comprising the peptides of Table 211 (Seq. ID. Nos. 2059-2063). In some embodiments, the donor cell source is HLA-B*18, and the LMP1 targeted T-cell subpopulation is primed and expanded with LMP1-derived peptides comprising the peptides of Table 211 (Seq. ID. Nos. 2059-2063) and at least one additional set of peptides based on the donor cell source HLA-B profile, wherein the at least one additional set of peptides are selected from the peptides of Tables 2088-210 and 212-214. In some embodiments, the LMP1-derived peptides also include one or more sets of HLA-A and HLA-DR restricted peptides selected from Tables 201-207 and 215-220 (Seq. ID Nos. 2009-2043 and 2079-2108).

TABLE 211
EBV Strain B95-8 LMP1 HLA-B*18 Epitope Peptides

| SEQ ID NO. | Sequence |
|---|---|
| 2059 | DEHHHDDSL |
| 2060 | DLILLIIAL |
| 2061 | NEGRHHLL |
| 2062 | DLLWLLLF |
| 2063 | EEVENKGG |

In some embodiments, the donor cell source is HLA-B*27:05, and the LMP1 targeted T-cell subpopulation is primed and expanded with one or more LMP1-derived peptides selected from Table 212 (Seq. ID. Nos. 2064-2068). In some embodiments, the donor cell source is HLA-B*27:05, and the LMP1 targeted T-cell subpopulation is primed and expanded with LMP1-derived peptides selected from Table 212 (Seq. ID. Nos. 2064-2068). In some embodiments, the donor cell source is HLA-B*27:05, and the LMP1 targeted T-cell subpopulation is primed and expanded with LMP1-derived peptides comprising the peptides of Table 212 (Seq. ID. Nos. 2064-2068). In some embodiments, the donor cell source is HLA-B*27:05, and the LMP1 targeted T-cell subpopulation is primed and expanded with LMP1-derived peptides comprising the peptides of Table 212 (Seq. ID. Nos. 2064-2068) and at least one additional set of peptides based on the donor cell source HLA-B profile, wherein the at least one additional set of peptides are selected from the peptides of Tables 208-211 and 213-214. In some embodiments, the LMP1-derived peptides also include one or more sets of HLA-A and HLA-DR restricted peptides selected from Tables 201-207 and 215-220 (Seq. ID Nos. 2009-2043 and 2079-2108).

TABLE 212
EBV Strain B95-8 LMP1 HLA-B*27:05
Epitope Peptides

| SEQ ID NO. | Sequence |
|---|---|
| 2064 | WRLGATIWQL |
| 2065 | PRGPPLSSSL |
| 2066 | RRPPRGPPL |
| 2067 | ERGPPGPRR |
| 2068 | FRRDLLCPL |

In some embodiments, the donor cell source is HLA-B*35:01, and the LMP1 targeted T-cell subpopulation is primed and expanded with one or more LMP1-derived peptides selected from Table 213 (Seq. ID. Nos. 2069-2073). In some embodiments, the donor cell source is HLA-B*35:01, and the LMP1 targeted T-cell subpopulation is primed and expanded with LMP1-derived peptides selected from Table 213 (Seq. ID. Nos. 2069-2073). In some embodiments, the donor cell source is HLA-B*35:01, and the LMP1 targeted T-cell subpopulation is primed and expanded with LMP1-derived peptides comprising the peptides of Table 213 (Seq. ID. Nos. 2069-2073). In some embodiments, the donor cell source is HLA-B*35:01, and the LMP1 targeted T-cell subpopulation is primed and expanded with LMP1-derived peptides comprising the peptides of Table 213 (Seq. ID. Nos. 2069-2073) and at least one additional set of peptides based on the donor cell source HLA-B profile, wherein the at least one additional set of peptides are selected from the peptides of Tables 208-212 and 214. In some embodiments, the LMP1-derived peptides also include one or more sets of HLA-A and HLA-DR restricted peptides selected from Tables 201-207 and 215-220 (Seq. ID Nos. 2009-2043 and 2079-2108).

TABLE 213
EBV Strain B95-8 LMP1 HLA-B*35:01
Epitope Peptides

| SEQ ID NO. | Sequence |
|---|---|
| 2069 | PPLSSSLGL |
| 2070 | DPHLPTLLL |
| 2071 | GPPLCSQNL |
| 2072 | CPLGALCILL |
| 2073 | DPHGPVQLSY |

In some embodiments, the donor cell source is HLA-B*58:02, and the LMP1 targeted T-cell subpopulation is primed and expanded with one or more LMP1-derived peptides selected from Table 214 (Seq. ID. Nos. 2074-2078). In some embodiments, the donor cell source is HLA-B*58:02, and the LMP1 targeted T-cell subpopulation is primed and expanded with LMP1-derived peptides selected from Table 214 (Seq. ID. Nos. 2074-2078). In some embodiments, the donor cell source is HLA-B*58:02, and the LMP1 targeted T-cell subpopulation is primed and expanded with LMP1-derived peptides comprising the peptides of Table 214 (Seq. ID. Nos. 2074-2078). In some embodiments, the donor cell source is HLA-B*58:02, and the LMP1 targeted T-cell subpopulation is primed and expanded with LMP1-derived peptides comprising the peptides of Table 214 (Seq. ID. Nos. 2074-2078) and at least one additional set of peptides based on the donor cell source HLA-B profile, wherein the at least one additional set of peptides are selected from the peptides of Tables 208-213. In some embodiments, the LMP1-derived peptides also include one or more sets of HLA-A and HLA-DR restricted peptides selected from Tables 201-207 and 215-220 (Seq. ID Nos. 2009-2043 and 2079-2108).

TABLE 214
EBV Strain B95-8 LMP1 HLA-B*58:02
Epitope Peptides

| SEQ ID NO. | Sequence |
|---|---|
| 2074 | SSLGLALLL |
| 2075 | ITLLLIALW |
| 2076 | LSSSLGLAL |
| 2077 | SSSLGLALLL |
| 2078 | NSNEGRHHLL |

In some embodiments, the donor cell source is HLA-DRB1*0101, and the LMP1 targeted T-cell subpopulation is primed and expanded with one or more LMP1-derived peptides selected from Table 215 (Seq. ID. Nos. 2079-2083). In some embodiments, the donor cell source is HLA-DRB1*0101, and the LMP1 targeted T-cell subpopulation is primed and expanded with LMP1-derived peptides selected from Table 215 (Seq. ID. Nos. 2079-2083). In some embodiments, the donor cell source is HLA-DRB1*0101, and the LMP1 targeted T-cell subpopulation is primed and expanded with LMP1-derived peptides comprising the peptides of Table 215 (Seq. ID. Nos. 2079-2083). In some embodiments, the donor cell source is HLA-DRB1*0101, and the LMP1 targeted T-cell subpopulation is primed and expanded with LMP1-derived peptides comprising the peptides of Table 215 (Seq. ID. Nos. 2079-2083) and at least one additional set of peptides based on the donor cell source HLA-DR profile, wherein the at least one additional set of peptides are selected from the peptides of Tables 216-220. In some embodiments, the LMP1-derived peptides also include one or more sets of HLA-A and HLA-B restricted peptides selected from Tables 201-214 (Seq. ID Nos. 2009-2078).

TABLE 215
EBV Strain B95-8 LMP1 HLA-DRB1*0101
Epitope Peptides

| SEQ ID NO. | Sequence |
|---|---|
| 2079 | LALLLLLLALLFWLY |
| 2080 | RDLLCPLGALCILLL |
| 2081 | LIALWNLHGQALFLG |
| 2082 | GATIWQLLAFFLAFF |
| 2083 | LGIVLFIFGCLLVLG |

In some embodiments, the donor cell source is HLA-DRB1*0301, and the LMP1 targeted T-cell subpopulation is primed and expanded with one or more LMP1-derived peptides selected from Table 216 (Seq. ID. Nos. 2084-2088). In some embodiments, the donor cell source is HLA-DRB1*0301, and the LMP1 targeted T-cell subpopulation is primed and expanded with LMP1-derived peptides selected from Table 216 (Seq. ID. Nos. 2084-2088). In some embodiments, the donor cell source is HLA-DRB1*0301, and the LMP1 targeted T-cell subpopulation is primed and expanded with LMP1-derived peptides comprising the peptides of Table 216 (Seq. ID. Nos. 2084-2088). In some embodiments, the donor cell source is HLA-DRB1*0301, and the LMP1 targeted T-cell subpopulation is primed and expanded with LMP1-derived peptides comprising the peptides of Table 216 (Seq. ID. Nos. 2084-2088) and at least one additional set of peptides based on the donor cell source HLA-DR profile, wherein the at least one additional set of peptides are selected from the peptides of Tables 215 and 217-220. In some embodiments, the LMP1-derived peptides also include one or more sets of HLA-A and HLA-B restricted peptides selected from Tables 201-214 (Seq. ID Nos. 2009-2078).

TABLE 216
EBV Strain B95-8 LMP1 HLA-DRB1*0301 (DR17)
Epitope Peptides

| SEQ ID NO. | Sequence |
|---|---|
| 2084 | WWTLLVDLLWLLLFL |
| 2085 | IFIFRRDLLCPLGAL |
| 2086 | IILIIFIFRRDLLCP |
| 2087 | ILIIFIFRRDLLCPL |
| 2088 | GLALLLLLLALLFWL |

In some embodiments, the donor cell source is HLA-DRB1*0401, and the LMP1 targeted T-cell subpopulation is primed and expanded with one or more LMP1-derived peptides selected from Table 217 (Seq. ID. Nos. 2089-2093). In some embodiments, the donor cell source is HLA-DRB1*0401, and the LMP1 targeted T-cell subpopulation is primed and expanded with LMP1-derived peptides selected from Table 217 (Seq. ID. Nos. 2089-2093). In some embodiments, the donor cell source is HLA-DRB1*0401, and the LMP1 targeted T-cell subpopulation is primed and expanded with LMP1-derived peptides comprising the peptides of Table 217 (Seq. ID. Nos. 2089-2093). In some embodiments, the donor cell source is HLA-DRB1*0401, and the LMP1 targeted T-cell subpopulation is primed and expanded with LMP1-derived peptides comprising the peptides of Table 217 (Seq. ID. Nos. 2089-2093) and at least one additional set of peptides based on the donor cell source HLA-DR profile, wherein the at least one additional set of peptides are selected from the peptides of Tables 215-216 and 218-220. In some embodiments, the LMP1-derived peptides also include one or more sets of HLA-A and HLA-B restricted peptides selected from Tables 201-214 (Seq. ID Nos. 2009-2078).

TABLE 217
EBV Strain B95-8 LMP1 HLA-DRB1*0401 (DR4Dw4)
Epitope Peptides

| SEQ ID NO. | Sequence |
|---|---|
| 2089 | LFWLYIVMSDWTGGA |
| 2090 | LYIVMSDWTGGALLV |
| 2091 | GGALLVLYSFALMLI |
| 2092 | IILIIFIFRRDLLCP |
| 2093 | ILIIFIFRRDLLCPL |

In some embodiments, the donor cell source is HLA-DRB1*0701, and the LMP1 targeted T-cell subpopulation is primed and expanded with one or more LMP1-derived peptides selected from Table 218 (Seq. ID. Nos. 2094-2098). In some embodiments, the donor cell source is HLA-DRB1*0701, and the LMP1 targeted T-cell subpopulation is primed and expanded with LMP1-derived peptides selected from Table 218 (Seq. ID. Nos. 2094-2098). In some embodiments, the donor cell source is HLA-DRB1*0701, and the LMP1 targeted T-cell subpopulation is primed and expanded with LMP1-derived peptides comprising the peptides of Table 218 (Seq. ID. Nos. 2094-2098). In some embodiments, the donor cell source is HLA-DRB1*0701, and the LMP1 targeted T-cell subpopulation is primed and expanded with LMP1-derived peptides comprising the peptides of Table 218 (Seq. ID. Nos. 2094-2098) and at least one additional set of peptides based on the donor cell source HLA-DR profile, wherein the at least one additional set of peptides are selected from the peptides of Tables 215-217 and 219-220. In some embodiments, the LMP1-derived peptides also include one or more sets of HLA-A and HLA-B restricted peptides selected from Tables 201-214 (Seq. ID Nos. 2009-2078).

TABLE 218
EBV Strain B95-8 LMP1 HLA-DRB1*0701
Epitope Peptides

| SEQ ID NO. | Sequence |
|---|---|
| 2094 | HDPLPHSPSDSAGND |
| 2095 | GGALLVLYSFALMLI |
| 2096 | LVLYSFALMLIIIIL |
| 2097 | LCILLLMITLLLIAL |
| 2098 | LWRLGATIWQLLAFF |

In some embodiments, the donor cell source is HLA-DRB1*1101, and the LMP1 targeted T-cell subpopulation is primed and expanded with one or more LMP1-derived peptides selected from Table 219 (Seq. ID. Nos. 2099-2103). In some embodiments, the donor cell source is HLA-DRB1*1101, and the LMP1 targeted T-cell subpopulation is primed and expanded with LMP1-derived peptides selected from Table 219 (Seq. ID. Nos. 2099-2103). In some embodiments, the donor cell source is HLA-DRB1*1101, and the LMP1 targeted T-cell subpopulation is primed and expanded with LMP1-derived peptides comprising the peptides of Table 219 (Seq. ID. 2099-2103). In some embodiments, the donor cell source is HLA-DRB1*1101, and the LMP1 targeted T-cell subpopulation is primed and expanded with LMP1-derived peptides comprising the peptides of Table 219 (Seq. ID. Nos. 2099-2103) and at least one additional set of peptides based on the donor cell source HLA-DR profile, wherein the at least one additional set of peptides are selected from the peptides of Tables 215-218 and 220. In some embodiments, the LMP1-derived peptides also include one or more sets of HLA-A and HLA-B restricted peptides selected from Tables 201-214 (Seq. ID Nos. 2009-2078).

TABLE 219
EBV Strain B95-8 LMP1 HLA-DRB1*1101
Epitope Peptides

| SEQ ID NO. | Sequence |
|---|---|
| 2099 | IYLLEMLWRLGATIW |
| 2100 | FWLYIVMSDWTGGAL |
| 2101 | ATIWQLLAFFLAFFL |
| 2102 | IILIIFIFRRDLLCP |
| 2103 | QNWWTLLVDLLWLLL |

In some embodiments, the donor cell source is HLA-DRB1*1501, and the LMP1 targeted T-cell subpopulation is primed and expanded with one or more LMP1-derived peptides selected from Table 220 (Seq. ID. Nos. 2104-2108). In some embodiments, the donor cell source is HLA-DRB 1*1501, and the LMP1 targeted T-cell subpopulation is primed and expanded with LMP1-derived peptides selected from Table 220 (Seq. ID. Nos. 2104-2108). In some embodiments, the donor cell source is HLA-DRB1*1501, and the LMP1 targeted T-cell subpopulation is primed and expanded with LMP1-derived peptides comprising the peptides of Table 220 (Seq. ID. Nos. 2104-2108). In some embodiments, the donor cell source is HLA-DRB1*1501, and the LMP1 targeted T-cell subpopulation is primed and expanded with LMP1-derived peptides comprising the peptides of Table 200 (Seq. ID. Nos. 2104-2108) and at least one additional set of peptides based on the donor cell source HLA-DR profile, wherein the at least one additional set of peptides are selected from the peptides of Tables 215-219. In some embodiments, the LMP1-derived peptides also include one or more sets of HLA-A and HLA-B restricted peptides selected from Tables 201-214 (Seq. ID Nos. 2009-2078).

TABLE 220
EBV Strain B95-8 LMP1 HLA-DRB1*1501 (DR2b)
Epitope Peptides

| SEQ ID NO. | Sequence |
|---|---|
| 2104 | WQLLAFFLAFFLDLI |
| 2105 | LLWLLLFLAILIWMY |
| 2106 | LLALLFWLYIVMSDW |
| 2107 | LLVLYSFALMLIIII |
| 2108 | GIVLFIFGCLLVLGI |

Epstein-Barr Virus (EBV) Strain B95-8 LMP2 Antigenic Peptides

In some embodiments, the MUSTANG composition includes Epstein-Barr Virus (EBV) Strain B95-8 LMP2 specific T-cells. LMP2 specific T-cells can be generated as described below using one or more antigenic peptides to LMP2. In some embodiments, the LMP2 specific T-cells are generated using one or more antigenic peptides to LMP2, or a modified or heteroclitic peptide derived from a LMP2 peptide. In some embodiments, LMP2 specific T-cells are generated using a LMP2 antigen library comprising a pool of peptides (for example 15mers) containing amino acid overlap (for example 11 amino acids of overlap) between each sequence formed by scanning the protein amino acid sequence SEQ. ID. No. 2109 (UniProt KB-P13285) for EBV Strain B95-8 LMP2:

MGSLEMVPMGAGPPSPGGDPDGYDGGNNSQYPSASGSSGNTPTPPNDEE
RESNEEPPPPYEDPYWGNGDRHSDYQPLGTQDQSLYLGLQHDGNDGLPP
PPYSPRDDSSQHIYEEAGRGSMNPVCLPVIVAPYLFWLAAIAASCFTAS
VSTVVTATGLALSLLLLAAVASSYAAAQRKLLTPVTVLTAVVTFFAICL
TWRIEDPPFNSLLFALLAAAGGLQGIYVLVMLVLLILAYRRRWRRLTVC
GGIMFLACVLVLIVDAVLQLSPLLGAVTVVSMTLLLLAFVLWLSSPGGL
GTLGAALLTLAAALALLASLILGTLNLTTMFLLMLLWTLVVLLICSSCS
SCPLSKILLARLFLYALALLLLASALIAGGSILQTNFKSLSSTEFIPNL
FCMLLLIVAGILFILAILTEWGSGNRTYGPVFMCLGGLLTMVAGAVWLT
VMSNTLLSAWILTAGFLIFLIGFALFGVIRCCRYCCYYCLTLESEERPP
TPYRNTV.

In some embodiments, the LMP2 specific T-cells are generated using one or more antigenic peptides to LMP2, or a modified or heteroclitic peptide derived from a LMP2 peptide. In some embodiments, the LMP2 specific T-cells are generated with peptides that recognize class I MHC molecules. In some embodiments, the LMP2 specific T-cells are generated with peptides that recognize class II MHC molecules. In some embodiments, the LMP2 specific T-cells are generated with peptides that recognize both class I and class II MHC molecules.

In some embodiments, the LMP2 peptides used to prime and expand a T-cell subpopulation includes specifically selected HLA-restricted peptides generated by determining the HLA profile of the donor source, and including peptides derived from LMP2 that best match the donor's HLA. In some embodiments, the LMP2 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting LMP2 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 221-227, the HLA-B peptides are selected from the peptides of Tables 228-234, and the HLA-DR peptides are selected from the peptides of Tables 235-240. For example, if the donor cell source has an HLA profile that is HLA-A*01/*02:01; HLA-B*15:01/*18; and HLA-DRB1*0101/*0301, then the LMP2 peptides used to prime and expand the LMP2 specific T-cell subpopulation are restricted to the specific HLA profile, and may include the peptides identified in Table 221 (Seq. ID. Nos. 2010-2014) for HLA-A*01; Table 222 (Seq. ID. Nos. 2115-2119) for HLA-A*02:01; Table 230 (Seq. ID. Nos. 2155-2159) for HLA-B*15:01; Table 231 (Seq. ID. Nos. 2160-2164) for HLA-B*18; Table 235 (Seq. ID. Nos. 2180-2184) for HLA-DRB1*0101; and Table 236 (Seq. ID. Nos. 2185-2189) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the LMP2 targeted T-cell subpopulation is primed and expanded with one or more LMP2-derived peptides selected from Table 221 (Seq. ID. Nos. 2110-2114). In some embodiments, the donor cell source is HLA-A*01, and the LMP2 targeted T-cell subpopulation is primed and expanded with LMP2-derived peptides selected from Table 221 (Seq. ID. Nos. 2110-2114). In some embodiments, the donor cell source is HLA-A*01, and the LMP2 targeted T-cell subpopulation is primed and expanded with LMP2-derived peptides comprising the peptides of from Table 221 (Seq. ID. Nos. 2110-2114). In some embodiments, the donor cell source is HLA-A*01, and the LMP2 targeted T-cell subpopulation is primed and expanded with LMP2-derived peptides comprising the peptides of from Table 221 (Seq. ID. Nos. 2110-2114) and at least one additional set of peptides based on the donor cell source HLA-A profile, wherein the at least one additional set of peptides are selected from the peptides of Tables 222-227. In some embodiments, the LMP2-derived peptides also include one or more sets of HLA-B and HLA-DR restricted peptides selected from Tables 228-240 (Seq. ID Nos. 2145-2209).

TABLE 221
EBV Strain B95-8 LMP2 HLA-A*01 Epitope Peptides

| SEQ ID NO. | Sequence |
|---|---|
| 2110 | RDDSSQHIY |
| 2111 | ESEERPPTPY |
| 2112 | GYDGGNNSQY |
| 2113 | GNDGLPPPPY |
| 2114 | LTEWGSGNRTY |

In some embodiments, the donor cell source is HLA-A*02:01, and the LMP2 targeted T-cell subpopulation is primed and expanded with one or more LMP2-derived peptides selected from Table 222 (Seq. ID. Nos. 2115-2119). In some embodiments, the donor cell source is HLA-A*02:01, and the LMP2 targeted T-cell subpopulation is primed and expanded with LMP2-derived peptides selected from Table 222 (Seq. ID. Nos. 2115-2119). In some embodiments, the donor cell source is HLA-A*02:01, and the LMP2 targeted T-cell subpopulation is primed and expanded with LMP2-derived peptides comprising the peptides of Table 222 (Seq. ID. Nos. 2115-2119). In some embodiments, the donor cell source is HLA-A*02:01, and the LMP2 targeted T-cell subpopulation is primed and expanded with LMP2-derived peptides comprising the peptides of Table 222 (Seq. ID. Nos. 2115-2119) and at least one additional set of peptides based on the donor cell source HLA-A profile, wherein the at least one additional set of peptides are selected from the peptides of Tables 221, and 223-227. In some embodiments, the LMP2-derived peptides also include one or more sets of HLA-B and HLA-DR restricted peptides selected from Tables 228-240 (Seq. ID Nos. 2145-2209).

TABLE 222
EBV Strain B95-8 LMP2 HLA-A*02:01
Epitope Peptides

| SEQ ID NO. | Sequence |
|---|---|
| 2115 | LLLAFVLWL |
| 2116 | FLLMLLWTL |
| 2117 | LLASLILGTL |
| 2118 | LLARLFLYAL |
| 2119 | FLIGFALFGV |

In some embodiments, the donor cell source is HLA-A*03, and the LMP2 targeted T-cell subpopulation is primed and expanded with one or more LMP2-derived peptides selected from Table 223 (Seq. ID. Nos. 2120-2124). In some embodiments, the donor cell source is HLA-A*03, and the LMP2 targeted T-cell subpopulation is primed and expanded with LMP2-derived peptides selected from Table 223 (Seq. ID. Nos. 2120-2124). In some embodiments, the donor cell source is HLA-A*03, and the LMP2 targeted T-cell subpopulation is primed and expanded with LMP2-derived peptides comprising the peptides of Table 223 (Seq. ID. 2120-2124). In some embodiments, the donor cell source is HLA-A*03, and the LMP2 targeted T-cell subpopulation is primed and expanded with LMP2-derived peptides comprising the peptides of Table 223 (Seq. ID. Nos. 2120-2124) and at least one additional set of peptides based on the donor cell source HLA-A profile, wherein the at least one additional set of peptides are selected from the peptides of Tables 221-222 and 224-227. In some embodiments, the LMP2-derived peptides also include one or more sets of HLA-B and HLA-DR restricted peptides selected from Tables 228-240 (Seq. ID Nos. 2145-2209).

TABLE 223
EBV Strain B95-8 LMP2 HLA-A*03 Epitope Peptides

| SEQ ID NO. | Sequence |
|---|---|
| 2120 | LLAAVASSY |
| 2121 | ALIAGGSIL |
| 2122 | SLLLLAAVA |
| 2123 | LLLAAVASSY |
| 2124 | QLSPLLGAVT |

In some embodiments, the donor cell source is HLA-A*11:01, and the LMP2 targeted T-cell subpopulation is primed and expanded with one or more LMP2-derived peptides selected from Table 224 (Seq. ID. Nos. 2125-2129). In some embodiments, the donor cell source is HLA-A*11:01, and the LMP2 targeted T-cell subpopulation is primed and expanded with LMP2-derived peptides selected from Table 224 (Seq. ID. Nos. 2125-2129). In some embodiments, the donor cell source is HLA-A*11:01, and the LMP2 targeted T-cell subpopulation is primed and expanded with LMP2-derived peptides comprising the peptides of Table 224 (Seq. ID. Nos. 2125-2129). In some embodiments, the donor cell source is HLA-A*11:01, and the LMP2 targeted T-cell subpopulation is primed and expanded with LMP2-derived peptides comprising the peptides of Table 224 (Seq. ID. Nos. 2125-2129), and at least one additional set of peptides based on the donor cell source HLA-A profile, wherein the at least one additional set of peptides are selected from the peptides of Tables 221-223 and 225-227. In some embodiments, the LMP2-derived peptides also include one or more sets of HLA-B and HLA-DR restricted peptides selected from Tables 228-240 (Seq. ID Nos. 2145-2209).

TABLE 224
EBV Strain B95-8 LMP2 HLA-A*11:01 Epitope Peptides

| SEQ ID NO. | Sequence |
|---|---|
| 2125 | SSYAAAQRK |
| 2126 | GSILQTNFK |
| 2127 | SSCSSCPLSK |
| 2128 | ASSYAAAQRK |
| 2129 | AVLQLSPLLG |

In some embodiments, the donor cell source is HLA-A*24:02, and the LMP2 targeted T-cell subpopulation is primed and expanded with one or more LMP2-derived peptides selected from Table 225 (Seq. ID. Nos. 2130-2134). In some embodiments, the donor cell source is HLA-A*24:02, and the LMP2 targeted T-cell subpopulation is primed and expanded with LMP2-derived peptides selected from Table 225 (Seq. ID. Nos. 2130-2134). In some embodiments, the donor cell source is HLA-A*24:02, and the LMP2 targeted T-cell subpopulation is primed and expanded with LMP2-derived peptides comprising the peptides of Table 225 (Seq. ID. Nos. 2130-2134). In some embodiments, the donor cell source is HLA-A*24:02, and the LMP2 targeted T-cell subpopulation is primed and expanded with LMP2-derived peptides comprising the peptides of Table 225 (Seq. ID. Nos. 2130-2134), and at least one additional set of peptides based on the donor cell source HLA-A profile, wherein the at least one additional set of peptides are selected from the peptides of Tables 221-224 and 226-227. In some embodiments, the LMP2-derived peptides also include one or more sets of HLA-B and HLA-DR restricted peptides selected from Tables 228-240 (Seq. ID Nos. 2145-2209).

TABLE 225
EBV Strain B95-8 LMP2 HLA-A*24:02 Epitope Peptides

| SEQ ID NO. | Sequence |
|---|---|
| 2130 | TYGPVFMCL |
| 2131 | PYLFWLAAI |
| 2132 | SYAAAQRKLL |
| 2133 | IYVLVMLVLL |
| 2134 | MFLACVLVLI |

In some embodiments, the donor cell source is HLA-A*26, and the LMP2 targeted T-cell subpopulation is primed and expanded with one or more LMP2-derived peptides selected from Table 226 (Seq. ID. Nos. 2135-2139). In some embodiments, the donor cell source is HLA-A*26, and the LMP2 targeted T-cell subpopulation is primed and expanded with LMP2-derived peptides selected from Table 226 (Seq. ID. Nos. 2135-2139). In some embodiments, the donor cell source is HLA-A*26, and the LMP2 targeted T-cell subpopulation is primed and expanded with LMP2-derived peptides comprising the peptides of Table 226 (Seq. ID. Nos. 2135-2139). In some embodiments, the donor cell source is HLA-A*26, and the LMP2 targeted T-cell subpopulation is primed and expanded with LMP2-derived peptides comprising the peptides of Table 226 (Seq. ID. Nos. 2135-2139) and at least one additional set of peptides based on the donor cell source HLA-A profile, wherein the at least one additional set of peptides are selected from the peptides of Tables 221-225 and 227. In some embodiments, the LMP2-derived peptides also include one or more sets of HLA-B and HLA-DR restricted peptides selected from Tables 228-240 (Seq. ID Nos. 2145-2209).

TABLE 226
EBV Strain B95-8 LMP2 HLA-A*26 Epitope Peptides

| SEQ ID NO. | Sequence |
|---|---|
| 2135 | PVFMCLGGL |
| 2136 | DAVLQLSPL |
| 2137 | TVVSMTLLLL |
| 2138 | TVVTATGLAL |
| 2139 | VTVLTAVVTF |

In some embodiments, the donor cell source is HLA-A*68:01, and the LMP2 targeted T-cell subpopulation is primed and expanded with one or more LMP2-derived peptides selected from Table 227 (Seq. ID. Nos. 2140-2144). In some embodiments, the donor cell source is HLA-A*68:01, and the LMP2 targeted T-cell subpopulation is primed and expanded with LMP2-derived peptides selected from Table 227 (Seq. ID. Nos. 2140-2144). In some embodiments, the donor cell source is HLA-A*68:01, and the LMP2 targeted T-cell subpopulation is primed and expanded with LMP2-derived peptides comprising the peptides of Table 227 (Seq. ID. Nos. 2140-2144). In some embodiments, the donor cell source is HLA-A*68:01, and the LMP2 targeted T-cell subpopulation is primed and expanded with LMP2-derived peptides comprising the peptides of Table 227 (Seq. ID. Nos. 2140-2144), and at least one additional set of peptides based on the donor cell source HLA-A profile, wherein the at least one additional set of peptides are selected from the peptides of Tables 221-226. In some embodiments, the LMP2-derived peptides also include one or more sets of HLA-B and HLA-DR restricted peptides selected from Tables 228-240 (Seq. ID Nos. 2145-2209).

TABLE 227
EBV Strain B95-8 LMP2 HLA-A*68:01 Epitope Peptides

| SEQ ID NO. | Sequence |
|---|---|
| 2140 | AVASSYAAAQR |
| 2141 | VTFFAICLTWR |
| 2142 | LVLLILAYR |
| 2143 | PLSKILLAR |
| 2144 | VASSYAAAQR |

In some embodiments, the donor cell source is HLA-B*07:02, and the LMP2 targeted T-cell subpopulation is primed and expanded with one or more LMP2-derived peptides selected from Table 228 (Seq. ID. Nos. 2145-2149). In some embodiments, the donor cell source is HLA-B*07:02, and the LMP2 targeted T-cell subpopulation is primed and expanded with LMP2-derived peptides selected from Table 228 (Seq. ID. Nos. 2145-2149). In some embodiments, the donor cell source is HLA-B*07:02, and the LMP2 targeted T-cell subpopulation is primed and expanded with LMP2-derived peptides comprising the peptides of Table 228 (Seq. ID. Nos. 2145-2149). In some embodiments, the donor cell source is HLA-B*07:02, and the LMP2 targeted T-cell subpopulation is primed and expanded with LMP2-derived peptides comprising the peptides of Table 228 (Seq. ID. Nos. 2145-2149), and at least one additional set of peptides based on the donor cell source HLA-B profile, wherein the at least one additional set of peptides are selected from the peptides of Tables 229-234. In some embodiments, the LMP2-derived peptides also include one or more sets of HLA-A and HLA-DR restricted peptides selected from Tables 221-227 and 235-240 (Seq. ID Nos. 2110-2144 and 2180-2209).

TABLE 228
EBV Strain B95-8 LMP2 HLA-B*07:02 Epitope Peptides

| SEQ ID NO. | Sequence |
|---|---|
| 2145 | LPVIVAPYL |
| 2146 | APYLFWLAA |
| 2147 | IPNLFCMLLL |
| 2148 | QPLGTQDQSL |
| 2149 | SPGGLGTLGA |

In some embodiments, the donor cell source is HLA-B*08, and the LMP2 targeted T-cell subpopulation is primed and expanded with one or more LMP2-derived peptides selected from Table 229 (Seq. ID. Nos. 2150-2154). In some embodiments, the donor cell source is HLA-B*08, and the LMP2 targeted T-cell subpopulation is primed and expanded with LMP2-derived peptides selected from Table 229 (Seq. ID. Nos. 2150-2154). In some embodiments, the donor cell source is HLA-B*08, and the LMP2 targeted T-cell subpopulation is primed and expanded with LMP2-derived peptides comprising the peptides of Table 229 (Seq. ID. Nos. 2150-2154). In some embodiments, the donor cell source is HLA-B*08, and the LMP2 targeted T-cell subpopulation is primed and expanded with LMP2-derived peptides comprising the peptides of Table 229 (Seq. ID. Nos. 2150-2154) and at least one additional set of peptides based on the donor cell source HLA-B profile, wherein the at least one additional set of peptides are selected from the peptides of Tables 228 and 230-234. In some embodiments, the LMP2-derived peptides also include one or more sets of HLA-A and HLA-DR restricted peptides selected from Tables 221-227 and 235-240 (Seq. ID Nos. 2110-2144 and 2180-2209).

TABLE 229
EBV Strain B95-8 LMP2 HLA-B*08 Epitope Peptides

| SEQ ID NO. | Sequence |
|---|---|
| 2150 | CPLSKILL |
| 2151 | ILLARLFL |
| 2152 | AAAQRKLL |
| 2153 | AYRRRWRRL |
| 2154 | LARLFLYAL |

In some embodiments, the donor cell source is HLA-B*15:01, and the LMP2 targeted T-cell subpopulation is primed and expanded with one or more LMP2-derived peptides selected from Table 230 (Seq. ID. Nos. 2155-2159). In some embodiments, the donor cell source is HLA-B*15:01, and the LMP2 targeted T-cell subpopulation is primed and expanded with LMP2-derived peptides selected from Table 230 (Seq. ID. Nos. 2155-2159). In some embodiments, the donor cell source is HLA-B*15:01, and the LMP2 targeted T-cell subpopulation is primed and expanded with LMP2-derived peptides comprising the peptides of Table 230 (Seq. ID. Nos. 2155-2159). In some embodiments, the donor cell source is HLA-B*15:01, and the LMP2 targeted T-cell subpopulation is primed and expanded with LMP2-derived peptides comprising the peptides of Table 230 (Seq. ID. Nos. 2155-2159) and at least one additional set of peptides based on the donor cell source HLA-B profile, wherein the at least one additional set of peptides are selected from the peptides of Tables 228-229 and 231-234. In some embodiments, the LMP2-derived peptides also include one or more sets of HLA-A and HLA-DR restricted peptides selected from Tables 221-227 and 235-240 (Seq. ID Nos. 2110-2144 and 2180-2209).

TABLE 230
EBV Strain B95-8 LMP2 HLA-B*15:01 (B62)
Epitope Peptides

| SEQ ID NO. | Sequence |
|---|---|
| 2155 | MLVLLILAY |
| 2156 | CLPVIVAPY |
| 2157 | LLAAVASSY |
| 2158 | LLLAAVASSY |
| 2159 | RLTVCGGIMF |

In some embodiments, the donor cell source is HLA-B*18, and the LMP2 targeted T-cell subpopulation is primed and expanded with one or more LMP2-derived peptides selected from Table 231 (Seq. ID. Nos. 2160-2164). In some embodiments, the donor cell source is HLA-B*18, and the LMP2 targeted T-cell subpopulation is primed and expanded with LMP2-derived peptides selected from Table 231 (Seq. ID. Nos. 2160-2164). In some embodiments, the donor cell source is HLA-B*18, and the LMP2 targeted T-cell subpopulation is primed and expanded with LMP2-derived peptides comprising the peptides of Table 231 (Seq. ID. Nos. 2160-2164). In some embodiments, the donor cell source is HLA-B*18, and the LMP2 targeted T-cell subpopulation is primed and expanded with LMP2-derived peptides comprising the peptides of Table 231 (Seq. ID. Nos. 2160-2164) and at least one additional set of peptides based on the donor cell source HLA-B profile, wherein the at least one additional set of peptides are selected from the peptides of Tables 228-230 and 232-234. In some embodiments, the LMP2-derived peptides also include one or more sets of HLA-A and HLA-DR restricted peptides selected from Tables 221-227 and 235-240 (Seq. ID Nos. 2110-2144 and 2180-2209).

TABLE 231
EBV Strain B95-8 LMP2 HLA-B*18 Epitope Peptides

| SEQ ID NO. | Sequence |
|---|---|
| 2160 | SEERPPTPY |
| 2161 | IEDPPFNSL |
| 2162 | EERPPTPY |
| 2163 | TEFIPNLF |
| 2164 | NEEPPPPY |

In some embodiments, the donor cell source is HLA-B*27:05, and the LMP2 targeted T-cell subpopulation is primed and expanded with one or more LMP2-derived peptides selected from Table 232 (Seq. ID. Nos. 2165-2169). In some embodiments, the donor cell source is HLA-B*27:05, and the LMP2 targeted T-cell subpopulation is primed and expanded with LMP2-derived peptides selected from Table 232 (Seq. ID. Nos. 2165-2169). In some embodiments, the donor cell source is HLA-B*27:05, and the LMP2 targeted T-cell subpopulation is primed and expanded with LMP2-derived peptides comprising the peptides of Table 232 (Seq. ID. Nos. 2165-2169). In some embodiments, the donor cell source is HLA-B*27:05, and the LMP2 targeted T-cell subpopulation is primed and expanded with LMP2-derived peptides comprising the peptides of Table 232 (Seq. ID. Nos. 2165-2169) and at least one additional set of peptides based on the donor cell source HLA-B profile, wherein the at least one additional set of peptides are selected from the peptides of Tables 228-231 and 233-234. In some embodiments, the LMP2-derived peptides also include one or more sets of HLA-A and HLA-DR restricted peptides selected from Tables 221-227 and 235-240 (Seq. ID Nos. 2110-2144 and 2180-2209).

TABLE 232
EBV Strain B95-8 LMP2 HLA-B*27:05 Epitope Peptides

| SEQ ID NO. | Sequence |
|---|---|
| 2165 | ARLFLYALAL |
| 2166 | GRGSMNPVCL |
| 2167 | RRLTVCGGIM |
| 2168 | GGLQGIYVL |
| 2169 | CRYCCYYCL |

In some embodiments, the donor cell source is HLA-B*35:01, and the LMP2 targeted T-cell subpopulation is primed and expanded with one or more LMP2-derived peptides selected from Table 233 (Seq. ID. Nos. 2170-2174). In some embodiments, the donor cell source is HLA-B*35:01, and the LMP2 targeted T-cell subpopulation is primed and expanded with LMP2-derived peptides selected from Table 233 (Seq. ID. Nos. 2170-2174). In some embodiments, the donor cell source is HLA-B*35:01, and the LMP2 targeted T-cell subpopulation is primed and expanded with LMP2-derived peptides comprising the peptides of Table 233 (Seq. ID. Nos. 2170-2174). In some embodiments, the donor cell source is HLA-B*35:01, and the LMP2 targeted T-cell subpopulation is primed and expanded with LMP2-derived peptides comprising the peptides of Table 233 (Seq. ID. Nos. 2170-2174) and at least one additional set of peptides based on the donor cell source HLA-B profile, wherein the at least one additional set of peptides are selected from the peptides of Tables 228-232 and 234. In some embodiments, the LMP2-derived peptides also include one or more sets of HLA-A and HLA-DR restricted peptides selected from Tables 221-227 and 235-240 (Seq. ID Nos. 2110-2144 and 2180-2209).

TABLE 233
EBV Strain B95-8 LMP2 HLA-B*35:01 Epitope Peptides

| SEQ ID NO. | Sequence |
|---|---|
| 2170 | LPVIVAPYL |
| 2171 | SPGGDPDGY |
| 2172 | QPLGTQDQSL |
| 2173 | PPFNSLLFAL |
| 2174 | GPVFMCLGGL |

In some embodiments, the donor cell source is HLA-B*58:02, and the LMP2 targeted T-cell subpopulation is primed and expanded with one or more LMP2-derived peptides selected from Table 234 (Seq. ID. Nos. 2175-2179). In some embodiments, the donor cell source is HLA-B*58:02, and the LMP2 targeted T-cell subpopulation is primed and expanded with LMP2-derived peptides selected from Table 234 (Seq. ID. Nos. 2175-2179). In some embodiments, the donor cell source is HLA-B*58:02, and the LMP2 targeted T-cell subpopulation is primed and expanded with LMP2-derived peptides comprising the peptides of Table 234 (Seq. ID. Nos. 2175-2179). In some embodiments, the donor cell source is HLA-B*58:02, and the LMP2 targeted T-cell subpopulation is primed and expanded with LMP2-derived peptides comprising the peptides of Table 234 (Seq. ID. Nos. 2175-2179) and at least one additional set of peptides based on the donor cell source HLA-B profile, wherein the at least one additional set of peptides are selected from the peptides of Tables 228-233. In some embodiments, the LMP2-derived peptides also include one or more sets of HLA-A and HLA-DR restricted peptides selected from Tables 221-227 and 235-240 (Seq. ID Nos. 2110-2144 and 2180-2209).

TABLE 234
EBV Strain B95-8 LMP2 HLA-B*58:02 Epitope Peptides

| SEQ ID NO. | Sequence |
|---|---|
| 2175 | KSLSSTEFI |
| 2176 | SSCPLSKIL |
| 2177 | LSKILLARLF |
| 2178 | SSYAAAQRKL |
| 2179 | LSSPGGLGTL |

In some embodiments, the donor cell source is HLA-DRB1*0101, and the LMP2 targeted T-cell subpopulation is primed and expanded with one or more LMP2-derived peptides selected from Table 235 (Seq. ID. Nos. 2180-2184). In some embodiments, the donor cell source is HLA-DRB1*0101, and the LMP2 targeted T-cell subpopulation is primed and expanded with LMP2-derived peptides selected from Table 235 (Seq. ID. Nos. 2180-2184). In some embodiments, the donor cell source is HLA-DRB1*0101, and the LMP2 targeted T-cell subpopulation is primed and expanded with LMP2-derived peptides comprising the peptides of Table 235 (Seq. ID. Nos. 2180-2184). In some embodiments, the donor cell source is HLA-DRB1*0101, and the LMP2 targeted T-cell subpopulation is primed and expanded with LMP2-derived peptides comprising the peptides of Table 235 (Seq. ID. Nos. 2180-2184) and at least one additional set of peptides based on the donor cell source HLA-DR profile, wherein the at least one additional set of peptides are selected from the peptides of Tables 236-240. In some embodiments, the LMP2-derived peptides also include one or more sets of HLA-A and HLA-B restricted peptides selected from Tables 221-234 (Seq. ID Nos. 2110-2179).

TABLE 235
EBV Strain B95-8 LMP2 HLA-DRB1*0101
Epitope Peptides

| SEQ ID NO. | Sequence |
|---|---|
| 2180 | QTNFKSLSSTEFIPN |
| 2181 | ALSLLLLAAVASSYA |
| 2182 | PGGLGTLGAALLTLA |
| 2183 | CMLLLIVAGILFILA |
| 2184 | AGFLIFLIGFALFGV |

In some embodiments, the donor cell source is HLA-DRB1*0301, and the LMP2 targeted T-cell subpopulation is primed and expanded with one or more LMP2-derived peptides selected from Table 236 (Seq. ID. Nos. 2185-2189). In some embodiments, the donor cell source is HLA-DRB1*0301, and the LMP2 targeted T-cell subpopulation is primed and expanded with LMP2-derived peptides selected from Table 236 (Seq. ID. Nos. 2185-2189). In some embodiments, the donor cell source is HLA-DRB1*0301, and the LMP2 targeted T-cell subpopulation is primed and expanded with LMP2-derived peptides comprising the peptides of Table 236 (Seq. ID. Nos. 2185-2189). In some embodiments, the donor cell source is HLA-DRB1*0301, and the LMP2 targeted T-cell subpopulation is primed and expanded with LMP2-derived peptides comprising the peptides of Table 236 (Seq. ID. Nos. 2185-2189) and at least one additional set of peptides based on the donor cell source HLA-DR profile, wherein the at least one additional set of peptides are selected from the peptides of Tables 235 and 237-240. In some embodiments, the LMP2-derived peptides also include one or more sets of HLA-A and HLA-B restricted peptides selected from Tables 221-234 (Seq. ID Nos. 2110-2179).

TABLE 236
EBV Strain B95-8 LMP2 HLA-DRB1*0301 (DR17)
Epitope Peptides

| SEQ ID NO. | Sequence |
|---|---|
| 2185 | YLGLQHDGNDGLPPP |
| 2186 | VLVLIVDAVLQLSPL |
| 2187 | AVWLTVMSNTLLSAW |
| 2188 | YQPLGTQDQSLYLGL |
| 2189 | VLVMLVLLILAYRRR |

In some embodiments, the donor cell source is HLA-DRB1*0401, and the LMP2 targeted T-cell subpopulation is primed and expanded with one or more LMP2-derived peptides selected from Table 237 (Seq. ID. Nos. 2190-2194). In some embodiments, the donor cell source is HLA-DRB1*0401, and the LMP2 targeted T-cell subpopulation is primed and expanded with LMP2-derived peptides selected from Table 237 (Seq. ID. Nos. 2190-2194). In some embodiments, the donor cell source is HLA-DRB1*0401, and the LMP2 targeted T-cell subpopulation is primed and expanded with LMP2-derived peptides comprising the peptides of Table 237 (Seq. ID. Nos. 2190-2194). In some embodiments, the donor cell source is HLA-DRB1*0401, and the LMP2 targeted T-cell subpopulation is primed and expanded with LMP2-derived peptides comprising the peptides of Table 237 (Seq. ID. Nos. 2190-2194) and at least one additional set of peptides based on the donor cell source HLA-DR profile, wherein the at least one additional set of peptides are selected from the peptides of Tables 235-236 and 238-240. In some embodiments, the LMP2-derived peptides also include one or more sets of HLA-A and HLA-B restricted peptides selected from Tables 221-234 (Seq. ID Nos. 2110-2179).

TABLE 237
EBV Strain B95-8 LMP2 HLA-DRB1*0401 (DR4Dw4)
Epitope Peptides

| SEQ ID NO. | Sequence |
|---|---|
| 2190 | HSDYQPLGTQDQSLY |
| 2191 | QSLYLGLQHDGNDGL |
| 2192 | QHIYEEAGRGSMNPV |
| 2193 | ASSYAAAQRKLLTPV |
| 2194 | GAVWLTVMSNTLLSA |

In some embodiments, the donor cell source is HLA-DRB1*0701, and the LMP2 targeted T-cell subpopulation is primed and expanded with one or more LMP2-derived peptides selected from Table 238 (Seq. ID. Nos. 2195-2199). In some embodiments, the donor cell source is HLA-DRB1*0701, and the LMP2 targeted T-cell subpopulation is primed and expanded with LMP2-derived peptides selected from Table 238 (Seq. ID. Nos. 2195-2199). In some embodiments, the donor cell source is HLA-DRB1*0701, and the LMP2 targeted T-cell subpopulation is primed and expanded with LMP2-derived peptides comprising the peptides of Table 238 (Seq. ID. Nos. 2195-2199). In some embodiments, the donor cell source is HLA-DRB1*0701, and the LMP2 targeted T-cell subpopulation is primed and expanded with LMP2-derived peptides comprising the peptides of Table 238 (Seq. ID. Nos. 2195-2199) and at least one additional set of peptides based on the donor cell source HLA-DR profile, wherein the at least one additional set of peptides are selected from the peptides of Tables 235-237 and 239-240. In some embodiments, the LMP2-derived peptides also include one or more sets of HLA-A and HLA-B restricted peptides selected from Tables 221-224 (Seq. ID Nos. 2110-2179).

TABLE 238
EBV Strain B95-8 LMP2 HLA-DRB1*0701
Epitope Peptides

| SEQ ID NO. | Sequence |
|---|---|
| 2195 | ASCFTASVSTVVTAT |
| 2196 | ACVLVLIVDAVLQLS |
| 2197 | VTFFAICLTWRIEDP |
| 2198 | GAVWLTVMSNTLLSA |
| 2199 | LSAWILTAGFLIFLI |

In some embodiments, the donor cell source is HLA-DRB1*1101, and the LMP2 targeted T-cell subpopulation is primed and expanded with one or more LMP2-derived peptides selected from Table 239 (Seq. ID. Nos. 2200-2204). In some embodiments, the donor cell source is HLA-DRB1*1101, and the LMP2 targeted T-cell subpopulation is primed and expanded with LMP2-derived peptides selected from Table 239 (Seq. ID. Nos. 2200-2204). In some embodiments, the donor cell source is HLA-DRB1*1101, and the LMP2 targeted T-cell subpopulation is primed and expanded with LMP2-derived peptides comprising the peptides of Table 239 (Seq. ID. 2200-2204). In some embodiments, the donor cell source is HLA-DRB1*1101, and the LMP2 targeted T-cell subpopulation is primed and expanded with LMP2-derived peptides comprising the peptides of Table 239 (Seq. ID. Nos. 2200-2204) and at least one additional set of peptides based on the donor cell source HLA-DR profile, wherein the at least one additional set of peptides are selected from the peptides of Tables 235-238 and 240. In some embodiments, the LMP2-derived peptides also include one or more sets of HLA-A and HLA-B restricted peptides selected from Tables 221-234 (Seq. ID Nos. 2110-2179).

TABLE 239
EBV Strain B95-8 LMP2 HLA-DRB1*1101
Epitope Peptides

| SEQ ID NO. | Sequence |
|---|---|
| 2200 | QSLYLGLQHDGNDGL |
| 2201 | LTEWGSGNRTYGPVF |
| 2202 | PNLFCMLLLIVAGIL |
| 2203 | NRTYGPVFMCLGGLL |
| 2204 | VTVLTAVVTFFAICL |

In some embodiments, the donor cell source is HLA-DRB1*1501, and the LMP2 targeted T-cell subpopulation is primed and expanded with one or more LMP2-derived peptides selected from Table 240 (Seq. ID. Nos. 2205-2209). In some embodiments, the donor cell source is HLA-DRB1*1501, and the LMP2 targeted T-cell subpopulation is primed and expanded with LMP2-derived peptides selected from Table 240 (Seq. ID. Nos. 2205-2209). In some embodiments, the donor cell source is HLA-DRB1*1501, and the LMP2 targeted T-cell subpopulation is primed and expanded with LMP2-derived peptides comprising the peptides of Table 240 (Seq. ID. Nos. 2205-2209). In some embodiments, the donor cell source is HLA-DRB1*1501, and the LMP2 targeted T-cell subpopulation is primed and expanded with LMP2-derived peptides comprising the peptides of Table 240 (Seq. ID. Nos. 2205-2209) and at least one additional set of peptides based on the donor cell source HLA-DR profile, wherein the at least one additional set of peptides are selected from the peptides of Tables 235-239. In some embodiments, the LMP2-derived peptides also include one or more sets of HLA-A and HLA-B restricted peptides selected from Tables 221-234 (Seq. ID Nos. 2110-2179).

TABLE 240
EBV Strain B95-8 LMP2 HLA-DRB1*1501 (DR2b)
Epitope Peptides

| SEQ ID NO. | Sequence |
|---|---|
| 2205 | LTAVVTFFAICLTWR |
| 2206 | FLLMLLWTLVVLLIC |
| 2207 | LIFLIGFALFGVIRC |
| 2208 | LAILTEWGSGNRTYG |
| 2209 | LIGFALFGVIRCCRY |

Epstein-Barr Virus (EBV) Strain B95-8 EBNA1 Antigenic Peptides

In some embodiments, the MUSTANG composition includes Epstein-Barr Virus (EBV) Strain B95-8 EBNA1 specific T-cells. EBNA1 specific T-cells can be generated as described below using one or more antigenic peptides to EBNA1. In some embodiments, the EBNA1 specific T-cells are generated using one or more antigenic peptides to EBNA1, or a modified or heteroclitic peptide derived from a EBNA1 peptide. In some embodiments, EBNA1 specific T-cells are generated using a EBNA1 antigen library comprising a pool of peptides (for example 15mers) containing amino acid overlap (for example 11 amino acids of overlap) between each sequence formed by scanning the protein amino acid sequence SEQ. ID. No. 2210 (UniProt KB-P03211) for EBV Strain B95-8 EBNA1:

MSDEGPGTGPGNGLGEKGDTSGPEGSGGSGPQRRGGDNHGRGRGRGRGR
GGGRPGAPGGSGSGPRHRDGVRRPQKRPSCIGCKGTHGGTGAGAGAGGA
GAGGAGAGGGAGAGGGAGGAGGAGGAGAGGGAGAGGGAGGAGGAGAGGG
AGAGGGAGGAGAGGGAGGAGGAGAGGGAGAGGGAGGAGAGGGAGGAGGA
GAGGGAGAGGAGGAGGAGAGGAGAGGGAGGAGGAGAGGAGAGGAGAGGA
GAGGAGGAGAGGAGGAGAGGAGGAGAGGGAGGAGAGGGAGGAGAGGAGG
AGAGGAGGAGAGGAGGAGAGGGAGAGGAGAGGGGRGRGGSGGRGRGGSG
GRGRGGSGGRRGRGRERARGGSRERARGRGRGRGEKRPRSPSSQSSSSG
SPPRRPPPGRRPFFHPVGEADYFEYHQEGGPDGEPDVPPGAIEQGPADD
PGEGPSTGPRGQGDGGRRKKGGWFGKHRGQGGSNPKFENIAEGLRALLA
RSHVERTTDEGTWVAGVFVYGGSKTSLYNLRRGTALAIPQCRLTPLSRL
PFGMAPGPGPQPGPLRESIVCYFMVFLQTHIFAEVLKDAIKDLVMTKPA
PTCNIRVTVCSFDDGVDLPPWFPPMVEGAAAEGDDGDDGDEGGDGDEGE
EGQE.

In some embodiments, the EBNA1 specific T-cells are generated using one or more antigenic peptides to EBNA1, or a modified or heteroclitic peptide derived from a EBNA1 peptide. In some embodiments, the EBNA1 specific T-cells are generated with peptides that recognize class I MHC molecules. In some embodiments, the EBNA1 specific T-cells are generated with peptides that recognize class II MHC molecules. In some embodiments, the EBNA1 specific T-cells are generated with peptides that recognize both class I and class II MHC molecules.

In some embodiments, the EBNA1 peptides used to prime and expand a T-cell subpopulation includes specifically selected HLA-restricted peptides generated by determining the HLA profile of the donor source, and including peptides derived from EBNA1 that best match the donor's HLA. In some embodiments, the EBNA1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting EBNA1 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 241-247, the HLA-B peptides are selected from the peptides of Tables 248-254, and the HLA-DR peptides are selected from the peptides of Tables 255-260. For example, if the donor cell source has an HLA profile that is HLA-A*01/*02:01; HLA-B*15:01/*18; and HLA-DRB1*0101/*0301, then the EBNA1 peptides used to prime and expand the EBNA1 specific T-cell subpopulation are restricted to the specific HLA profile, and may include the peptides identified in Table 241 (Seq. ID. Nos. 2211-2215) for HLA-A*01; Table 242 (Seq. ID. Nos. 2216-2220) for HLA-A*02:01; Table 250 (Seq. ID. Nos. 2256-2260) for HLA-B*15:01; Table 251 (Seq. ID. Nos. 2261-2265) for HLA-B*18; Table 255 (Seq. ID. Nos. 2281-2285) for HLA-DRB1*0101; and Table 256 (Seq. ID. Nos. 2286-2290) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the EBNA1 targeted T-cell subpopulation is primed and expanded with one or more EBNA1-derived peptides selected from Table 241 (Seq. ID. Nos. 2211-2215). In some embodiments, the donor cell source is HLA-A*01, and the EBNA1 targeted T-cell subpopulation is primed and expanded with EBNA1-derived peptides selected from Table 241 (Seq. ID. Nos. 2211-2215). In some embodiments, the donor cell source is HLA-A*01, and the EBNA1 targeted T-cell subpopulation is primed and expanded with EBNA1-derived peptides comprising the peptides of from Table 241 (Seq. ID. Nos. 2211-2215). In some embodiments, the donor cell source is HLA-A*01, and the EBNA1 targeted T-cell subpopulation is primed and expanded with EBNA1-derived peptides comprising the peptides of from Table 241 (Seq. ID. Nos. 2211-2215) and at least one additional set of peptides based on the donor cell source HLA-A profile, wherein the at least one additional set of peptides are selected from the peptides of Tables 242-247. In some embodiments, the EBNA1-derived peptides also include one or more sets of HLA-B and HLA-DR restricted peptides selected from Tables 248-260 (Seq. ID Nos. 2246-2310).

TABLE 241
EBV Strain B95-8 EBNA1 HLA-A*01 Epitope Peptides

| SEQ ID NO. | Sequence |
|---|---|
| 2211 | VGEADYFEY |
| 2212 | TTDEGTWVA |
| 2213 | TWVAGVFVY |
| 2214 | GTWVAGVFVY |
| 2215 | FVYGGSKTSLY |

In some embodiments, the donor cell source is HLA-A*02:01, and the EBNA1 targeted T-cell subpopulation is primed and expanded with one or more EBNA1-derived peptides selected from Table 242 (Seq. ID. Nos. 2216-2220). In some embodiments, the donor cell source is HLA-A*02:01, and the EBNA1 targeted T-cell subpopulation is primed and expanded with EBNA1-derived peptides selected from Table 242 (Seq. ID. Nos. 2216-2220). In some embodiments, the donor cell source is HLA-A*02:01, and the EBNA1 targeted T-cell subpopulation is primed and expanded with EBNA1-derived peptides comprising the peptides of Table 242 (Seq. ID. Nos. 2216-2220). In some embodiments, the donor cell source is HLA-A*02:01, and the EBNA1 targeted T-cell subpopulation is primed and expanded with EBNA1-derived peptides comprising the peptides of Table 242 (Seq. ID. Nos. 2216-2220) and at least one additional set of peptides based on the donor cell source HLA-A profile, wherein the at least one additional set of peptides are selected from the peptides of Tables 241, and 243-247. In some embodiments, the EBNA1-derived peptides also include one or more sets of HLA-B and HLA-DR restricted peptides selected from Tables 248-260 (Seq. ID Nos. 2246-2310).

TABLE 242
EBV Strain B95-8 EBNA1 HLA-A*02:01
Epitope Peptides

| SEQ ID NO. | Sequence |
|---|---|
| 2216 | NIAEGLRAL |
| 2217 | ALAIPQCRL |
| 2218 | VLKDAIKDL |
| 2219 | FLQTHIFAEV |
| 2220 | AIPQCRLTPL |

In some embodiments, the donor cell source is HLA-A*03, and the EBNA1 targeted T-cell subpopulation is primed and expanded with one or more EBNA1-derived peptides selected from Table 243 (Seq. ID. Nos. 2221-2225). In some embodiments, the donor cell source is HLA-A*03, and the EBNA1 targeted T-cell subpopulation is primed and expanded with EBNA1-derived peptides selected from Table 243 (Seq. ID. Nos. 2221-2225). In some embodiments, the donor cell source is HLA-A*03, and the EBNA1 targeted T-cell subpopulation is primed and expanded with EBNA1-derived peptides comprising the peptides of Table 243 (Seq. ID. 2221-2225). In some embodiments, the donor cell source is HLA-A*03, and the EBNA1 targeted T-cell subpopulation is primed and expanded with EBNA1-derived peptides comprising the peptides of Table 243 (Seq. ID. Nos. 2221-2225) and at least one additional set of peptides based on the donor cell source HLA-A profile, wherein the at least one additional set of peptides are selected from the peptides of Tables 241-242 and 244-247. In some embodiments, the EBNA1-derived peptides also include one or more sets of HLA-B and HLA-DR restricted peptides selected from Tables 248-260 (Seq. ID Nos. 2246-2310).

TABLE 243
EBV Strain B95-8 EBNA1 HLA-A*03 Epitope Peptides

| SEQ ID NO. | Sequence |
|---|---|
| 2221 | AIKDLVMTK |
| 2222 | GVFVYGGSK |
| 2223 | ALLARSHVER |
| 2224 | RLTPLSRLPF |
| 2225 | GLRALLARSH |

In some embodiments, the donor cell source is HLA-A*11:01, and the EBNA1 targeted T-cell subpopulation is primed and expanded with one or more EBNA1-derived peptides selected from Table 244 (Seq. ID. Nos. 2226-2230). In some embodiments, the donor cell source is HLA-A*11:01, and the EBNA1 targeted T-cell subpopulation is primed and expanded with EBNA1-derived peptides selected from Table 244 (Seq. ID. Nos. 2226-2230). In some embodiments, the donor cell source is HLA-A*11:01, and the EBNA1 targeted T-cell subpopulation is primed and expanded with EBNA1-derived peptides comprising the peptides of Table 244 (Seq. ID. Nos. 2226-2230). In some embodiments, the donor cell source is HLA-A*11:01, and the EBNA1 targeted T-cell subpopulation is primed and expanded with EBNA1-derived peptides comprising the peptides of Table 244 (Seq. ID. Nos. 2226-2230), and at least one additional set of peptides based on the donor cell source HLA-A profile, wherein the at least one additional set of peptides are selected from the peptides of Tables 241-243 and 245-247. In some embodiments, the EBNA1-derived peptides also include one or more sets of HLA-B and HLA-DR restricted peptides selected from Tables 248-260 (Seq. ID Nos. 2246-2310).

TABLE 244
EBV Strain B95-8 EBNA1 HLA-A*11:01
Epitope Peptides

| SEQ ID NO. | Sequence |
|---|---|
| 2226 | GVFVYGGSK |
| 2227 | GSGSGPRHR |
| 2228 | QTHIFAEVLK |
| 2229 | ALLARSHVER |
| 2230 | GSKTSLYNLR |

In some embodiments, the donor cell source is HLA-A*24:02, and the EBNA1 targeted T-cell subpopulation is primed and expanded with one or more EBNA1-derived peptides selected from Table 245 (Seq. ID. Nos. 2231-2235). In some embodiments, the donor cell source is HLA-A*24:02, and the EBNA1 targeted T-cell subpopulation is primed and expanded with EBNA1-derived peptides selected from Table 245 (Seq. ID. Nos. 2231-2235). In some embodiments, the donor cell source is HLA-A*24:02, and the EBNA1 targeted T-cell subpopulation is primed and expanded with EBNA1-derived peptides comprising the peptides of Table 245 (Seq. ID. Nos. 2231-2235). In some embodiments, the donor cell source is HLA-A*24:02, and the EBNA1 targeted T-cell subpopulation is primed and expanded with EBNA1-derived peptides comprising the peptides of Table 245 (Seq. ID. Nos. 2231-2235), and at least one additional set of peptides based on the donor cell source HLA-A profile, wherein the at least one additional set of peptides are selected from the peptides of Tables 241-244 and 246-247. In some embodiments, the EBNA1-derived peptides also include one or more sets of HLA-B and HLA-DR restricted peptides selected from Tables 248-260 (Seq. ID Nos. 2246-2310).

TABLE 245
EBV Strain B95-8 EBNA1 HLA-A*24:02
Epitope Peptides

| SEQ ID NO. | Sequence |
|---|---|
| 2231 | VYGGSKTSL |
| 2232 | LYNLRRGTAL |
| 2233 | KFENIAEGL |
| 2234 | IFAEVLKDAI |
| 2235 | YFMVFLQTHI |

In some embodiments, the donor cell source is HLA-A*26, and the EBNA1 targeted T-cell subpopulation is primed and expanded with one or more EBNA1-derived peptides selected from Table 246 (Seq. ID. Nos. 2236-2240). In some embodiments, the donor cell source is HLA-A*26, and the EBNA1 targeted T-cell subpopulation is primed and expanded with EBNA1-derived peptides selected from Table 246 (Seq. ID. Nos. 2236-2240). In some embodiments, the donor cell source is HLA-A*26, and the EBNA1 targeted T-cell subpopulation is primed and expanded with EBNA1-derived peptides comprising the peptides of Table 246 (Seq. ID. Nos. 2236-2240). In some embodiments, the donor cell source is HLA-A*26, and the EBNA1 targeted T-cell subpopulation is primed and expanded with EBNA1-derived peptides comprising the peptides of Table 246 (Seq. ID. Nos. 2236-2240) and at least one additional set of peptides based on the donor cell source HLA-A profile, wherein the at least one additional set of peptides are selected from the peptides of Tables 241-245 and 247. In some embodiments, the EBNA1-derived peptides also include one or more sets of HLA-B and HLA-DR restricted peptides selected from Tables 248-260 (Seq. ID Nos. 2246-2310).

TABLE 246
EBV Strain B95-8 EBNA1 HLA-A*26 Epitope Peptides

| SEQ ID NO. | Sequence |
|---|---|
| 2236 | MVFLQTHIF |
| 2237 | DGVDLPPWF |
| 2238 | EVLKDAIKDL |
| 2239 | ENIAEGLRAL |
| 2240 | DVPPGAIEQG |

In some embodiments, the donor cell source is HLA-A*68:01, and the EBNA1 targeted T-cell subpopulation is primed and expanded with one or more EBNA1-derived peptides selected from Table 247 (Seq. ID. Nos. 2241-2245). In some embodiments, the donor cell source is HLA-A*68:01, and the EBNA1 targeted T-cell subpopulation is primed and expanded with EBNA1-derived peptides selected from Table 247 (Seq. ID. Nos. 2241-2245). In some embodiments, the donor cell source is HLA-A*68:01, and the EBNA1 targeted T-cell subpopulation is primed and expanded with EBNA1-derived peptides comprising the peptides of Table 247 (Seq. ID. Nos. 2241-2245). In some embodiments, the donor cell source is HLA-A*68:01, and the EBNA1 targeted T-cell subpopulation is primed and expanded with EBNA1-derived peptides comprising the peptides of Table 247 (Seq. ID. Nos. 2241-2245), and at least one additional set of peptides based on the donor cell source HLA-A profile, wherein the at least one additional set of peptides are selected from the peptides of Tables 241-246. In some embodiments, the EBNA1-derived peptides also include one or more sets of HLA-B and HLA-DR restricted peptides selected from Tables 248-260 (Seq. ID Nos. 2246-2310).

TABLE 247
EBV Strain B95-8 EBNA1 HLA-A*68:01
Epitope Peptides

| SEQ ID NO. | Sequence |
|---|---|
| 2241 | RALLARSHVER |
| 2242 | GTALAIPQCR |
| 2243 | QTHIFAEVLK |
| 2244 | DAIKDLVMTK |
| 2245 | MTKPAPTCNIR |

In some embodiments, the donor cell source is HLA-B*07:02, and the EBNA1 targeted T-cell subpopulation is primed and expanded with one or more EBNA1-derived peptides selected from Table 248 (Seq. ID. Nos. 2246-2250). In some embodiments, the donor cell source is HLA-B*07:02, and the EBNA1 targeted T-cell subpopulation is primed and expanded with EBNA1-derived peptides selected from Table 248 (Seq. ID. Nos. 2246-2250). In some embodiments, the donor cell source is HLA-B*07:02, and the EBNA1 targeted T-cell subpopulation is primed and expanded with EBNA1-derived peptides comprising the peptides of Table 248 (Seq. ID. Nos. 2246-2250). In some embodiments, the donor cell source is HLA-B*07:02, and the EBNA1 targeted T-cell subpopulation is primed and expanded with EBNA1-derived peptides comprising the peptides of Table 248 (Seq. ID. Nos. 2246-2250), and at least one additional set of peptides based on the donor cell source HLA-B profile, wherein the at least one additional set of peptides are selected from the peptides of Tables 249-254. In some embodiments, the EBNA1-derived peptides also include one or more sets of HLA-A and HLA-DR restricted peptides selected from Tables 241-247 and 255-260 (Seq. ID Nos. 2211-2245 and 2281-2310).

TABLE 248
EBV Strain B95-8 EBNA1 HLA-B*07:02
Epitope Peptides

| SEQ ID NO. | Sequence |
|---|---|
| 2246 | IPQCRLTPL |
| 2247 | GPGPQPGPL |
| 2248 | EPDVPPGAI |
| 2249 | GPGTGPGNGL |
| 2250 | RPPPGRRPFF |

In some embodiments, the donor cell source is HLA-B*08, and the EBNA1 targeted T-cell subpopulation is primed and expanded with one or more EBNA1-derived peptides selected from Table 249 (Seq. ID. Nos. 2251-2255). In some embodiments, the donor cell source is HLA-B*08, and the EBNA1 targeted T-cell subpopulation is primed and expanded with EBNA1-derived peptides selected from Table 249 (Seq. ID. Nos. 2251-2255). In some embodiments, the donor cell source is HLA-B*08, and the EBNA1 targeted T-cell subpopulation is primed and expanded with EBNA1-derived peptides comprising the peptides of Table 249 (Seq. ID. Nos. 2251-2255). In some embodiments, the donor cell source is HLA-B*08, and the EBNA1 targeted T-cell subpopulation is primed and expanded with EBNA1-derived peptides comprising the peptides of Table 249 (Seq. ID. Nos. 2251-2255) and at least one additional set of peptides based on the donor cell source HLA-B profile, wherein the at least one additional set of peptides are selected from the peptides of Tables 248 and 250-254. In some embodiments, the EBNA1-derived peptides also include one or more sets of HLA-A and HLA-DR restricted peptides selected from Tables 241-247 and 255-260 (Seq. ID Nos. 2211-2245 and 2281-2310).

TABLE 249
EBV Strain B95-8 EBNA1 HLA-B*08 Epitope Peptides

| SEQ ID NO. | Sequence |
|---|---|
| 2251 | NLRRGTAL |
| 2252 | GPRHRDGV |
| 2253 | VLKDAIKDL |
| 2254 | IPQCRLTPL |
| 2255 | GRRKKGGWF |

In some embodiments, the donor cell source is HLA-B*15:01, and the EBNA1 targeted T-cell subpopulation is primed and expanded with one or more EBNA1-derived peptides selected from Table 250 (Seq. ID. Nos. 2256-2260). In some embodiments, the donor cell source is HLA-B*15:01, and the EBNA1 targeted T-cell subpopulation is primed and expanded with EBNA1-derived peptides selected from Table 250 (Seq. ID. Nos. 2256-2260). In some embodiments, the donor cell source is HLA-B*15:01, and the EBNA1 targeted T-cell subpopulation is primed and expanded with EBNA1-derived peptides comprising the peptides of Table 250 (Seq. ID. Nos. 2256-2260). In some embodiments, the donor cell source is HLA-B*15:01, and the EBNA1 targeted T-cell subpopulation is primed and expanded with EBNA1-derived peptides comprising the peptides of Table 250 (Seq. ID. Nos. 2256-2260) and at least one additional set of peptides based on the donor cell source HLA-B profile, wherein the at least one additional set of peptides are selected from the peptides of Tables 248-249 and 251-254. In some embodiments, the EBNA1-derived peptides also include one or more sets of HLA-A and HLA-DR restricted peptides selected from Tables 241-247 and 255-260 (Seq. ID Nos. 2211-2245 and 2281-2310).

TABLE 250
EBV Strain B95-8 EBNA1 HLA-B*15:01 (B62)
Epitope Peptides

| SEQ ID NO. | Sequence |
|---|---|
| 2256 | PLRESIVCY |
| 2257 | RLTPLSRLPF |
| 2258 | GQGGSNPKF |
| 2259 | PVGEADYFEY |
| 2260 | MVFLQTHIF |

In some embodiments, the donor cell source is HLA-B*18, and the EBNA1 targeted T-cell subpopulation is primed and expanded with one or more EBNA1-derived peptides selected from Table 251 (Seq. ID. Nos. 2261-2265). In some embodiments, the donor cell source is HLA-B*18, and the EBNA1 targeted T-cell subpopulation is primed and expanded with EBNA1-derived peptides selected from Table 251 (Seq. ID. Nos. 2261-2265). In some embodiments, the donor cell source is HLA-B*18, and the EBNA1 targeted T-cell subpopulation is primed and expanded with EBNA1-derived peptides comprising the peptides of Table 251 (Seq. ID. Nos. 2261-2265). In some embodiments, the donor cell source is HLA-B*18, and the EBNA1 targeted T-cell subpopulation is primed and expanded with EBNA1-derived peptides comprising the peptides of Table 251 (Seq. ID. Nos. 2261-2265) and at least one additional set of peptides based on the donor cell source HLA-B profile, wherein the at least one additional set of peptides are selected from the peptides of Tables 248-250 and 252-254. In some embodiments, the EBNA1-derived peptides also include one or more sets of HLA-A and HLA-DR restricted peptides selected from Tables 241-247 and 255-260 (Seq. ID Nos. 2211-2245 and 2281-2310).

TABLE 251
EBV Strain B95-8 EBNA1 HLA-B*18 Epitope Peptides

| SEQ ID NO. | Sequence |
|---|---|
| 2261 | RESIVCYF |
| 2262 | GEADYFEY |
| 2263 | FENIAEGL |
| 2264 | AEGLRALL |
| 2265 | DGVDLPPWF |

In some embodiments, the donor cell source is HLA-B*27:05, and the EBNA1 targeted T-cell subpopulation is primed and expanded with one or more EBNA1-derived peptides selected from Table 252 (Seq. ID. Nos. 2266-2270). In some embodiments, the donor cell source is HLA-B*27:05, and the EBNA1 targeted T-cell subpopulation is primed and expanded with EBNA1-derived peptides selected from Table 252 (Seq. ID. Nos. 2266-2270). In some embodiments, the donor cell source is HLA-B*27:05, and the EBNA1 targeted T-cell subpopulation is primed and expanded with EBNA1-derived peptides comprising the peptides of Table 252 (Seq. ID. Nos. 2266-2270). In some embodiments, the donor cell source is HLA-B*27:05, and the EBNA1 targeted T-cell subpopulation is primed and expanded with EBNA1-derived peptides comprising the peptides of Table 252 (Seq. ID. Nos. 2266-2270) and at least one additional set of peptides based on the donor cell source HLA-B profile, wherein the at least one additional set of peptides are selected from the peptides of Tables 248-251 and 253-254. In some embodiments, the EBNA1-derived peptides also include one or more sets of HLA-A and HLA-DR restricted peptides selected from Tables 241-247 and 255-260 (Seq. ID Nos. 2211-2245 and 2281-2310).

TABLE 252
EBV Strain B95-8 EBNA1 HLA-B*27:05
Epitope Peptides

| SEQ ID NO. | Sequence |
|---|---|
| 2266 | RRKKGGWFGK |
| 2267 | GRGGSGGRGR |
| 2268 | GRGGSGGRR |
| 2269 | RRGGDNHGR |
| 2270 | CRLTPLSRL |

In some embodiments, the donor cell source is HLA-B*35:01, and the EBNA1 targeted T-cell subpopulation is primed and expanded with one or more EBNA1-derived peptides selected from Table 253 (Seq. ID. Nos. 2271-2275). In some embodiments, the donor cell source is HLA-B*35:01, and the EBNA1 targeted T-cell subpopulation is primed and expanded with EBNA1-derived peptides selected from Table 253 (Seq. ID. Nos. 2271-2275). In some embodiments, the donor cell source is HLA-B*35:01, and the EBNA1 targeted T-cell subpopulation is primed and expanded with EBNA1-derived peptides comprising the peptides of Table 253 (Seq. ID. Nos. 2271-2275). In some embodiments, the donor cell source is HLA-B*35:01, and the EBNA1 targeted T-cell subpopulation is primed and expanded with EBNA1-derived peptides comprising the peptides of Table 253 (Seq. ID. Nos. 2271-2275) and at least one additional set of peptides based on the donor cell source HLA-B profile, wherein the at least one additional set of peptides are selected from the peptides of Tables 248-252 and 254. In some embodiments, the EBNA1-derived peptides also include one or more sets of HLA-A and HLA-DR restricted peptides selected from Tables 241-247 and 255-260 (Seq. ID Nos. 2211-2245 and 2281-2310).

TABLE 253
EBV Strain B95-8 EBNA1 HLA-B*35:01
Epitope Peptides

| SEQ ID NO. | Sequence |
|---|---|
| 2271 | HPVGEADYF |
| 2272 | IPQCRLTPL |
| 2273 | GPGPQPGPL |
| 2274 | GPLRESIVCY |
| 2275 | GPGTGPGNGL |

In some embodiments, the donor cell source is HLA-B*58:02, and the EBNA1 targeted T-cell subpopulation is primed and expanded with one or more EBNA1-derived peptides selected from Table 254 (Seq. ID. Nos. 2276-2280). In some embodiments, the donor cell source is HLA-B*58:02, and the EBNA1 targeted T-cell subpopulation is primed and expanded with EBNA1-derived peptides selected from Table 254 (Seq. ID. Nos. 2276-2280). In some embodiments, the donor cell source is HLA-B*58:02, and the EBNA1 targeted T-cell subpopulation is primed and expanded with EBNA1-derived peptides comprising the peptides of Table 254 (Seq. ID. Nos. 2276-2280). In some embodiments, the donor cell source is HLA-B*58:02, and the EBNA1 targeted T-cell subpopulation is primed and expanded with EBNA1-derived peptides comprising the peptides of Table 254 (Seq. ID. Nos. 2276-2280) and at least one additional set of peptides based on the donor cell source HLA-B profile, wherein the at least one additional set of peptides are selected from the peptides of Tables 248-253. In some embodiments, the EBNA1-derived peptides also include one or more sets of HLA-A and HLA-DR restricted peptides selected from Tables 241-247 and 255-260 (Seq. ID Nos. 2211-2245 and 2281-2310).

TABLE 254
EBV Strain B95-8 EBNA1 HLA-B*58:02
Epitope Peptides

| SEQ ID NO. | Sequence |
|---|---|
| 2276 | GSNPKFENI |
| 2277 | GSKTSLYNL |
| 2278 | IAEGLRALL |
| 2279 | ESIVCYFMVF |
| 2280 | MTKPAPTCNI |

In some embodiments, the donor cell source is HLA-DRB1*0101, and the EBNA1 targeted T-cell subpopulation is primed and expanded with one or more EBNA1-derived peptides selected from Table 255 (Seq. ID. Nos. 2281-2285). In some embodiments, the donor cell source is HLA-DRB1*0101, and the EBNA1 targeted T-cell subpopulation is primed and expanded with EBNA1-derived peptides selected from Table 255 (Seq. ID. Nos. 2281-2285). In some embodiments, the donor cell source is HLA-DRB1*0101, and the EBNA1 targeted T-cell subpopulation is primed and expanded with EBNA1-derived peptides comprising the peptides of Table 255 (Seq. ID. Nos. 2281-2285). In some embodiments, the donor cell source is HLA-DRB1*0101, and the EBNA1 targeted T-cell subpopulation is primed and expanded with EBNA1-derived peptides comprising the peptides of Table 255 (Seq. ID. Nos. 2281-2285) and at least one additional set of peptides based on the donor cell source HLA-DR profile, wherein the at least one additional set of peptides are selected from the peptides of Tables 256-260. In some embodiments, the EBNA1-derived peptides also include one or more sets of HLA-A and HLA-B restricted peptides selected from Tables 241-254 (Seq. ID Nos. 2211-2280).

TABLE 255
EBV Strain B95-8 EBNA1 HLA-DRB1*0101
Epitope Peptides

| SEQ ID NO. | Sequence |
|---|---|
| 2281 | CYFMVFLQTHIFAEV |
| 2282 | TSLYNLRRGTALAIP |
| 2283 | RLPFGMAPGPGPQPG |
| 2284 | AEGLRALLARSHVER |
| 2285 | AGVFVYGGSKTSLYN |

In some embodiments, the donor cell source is HLA-DRB1*0301, and the EBNA1 targeted T-cell subpopulation is primed and expanded with one or more EBNA1-derived peptides selected from Table 256 (Seq. ID. Nos. 2286-2290). In some embodiments, the donor cell source is HLA-DRB 1*0301, and the EBNA1 targeted T-cell subpopulation is primed and expanded with EBNA1-derived peptides selected from Table 256 (Seq. ID. Nos. 2286-2290). In some embodiments, the donor cell source is HLA-DRB1*0301, and the EBNA1 targeted T-cell subpopulation is primed and expanded with EBNA1-derived peptides comprising the peptides of Table 256 (Seq. ID. Nos. 2286-2290). In some embodiments, the donor cell source is HLA-DRB1*0301, and the EBNA1 targeted T-cell subpopulation is primed and expanded with EBNA1-derived peptides comprising the peptides of Table 256 (Seq. ID. Nos. 2286-2290) and at least one additional set of peptides based on the donor cell source HLA-DR profile, wherein the at least one additional set of peptides are selected from the peptides of Tables 255 and 257-260. In some embodiments, the EBNA1-derived peptides also include one or more sets of HLA-A and HLA-B restricted peptides selected from Tables 241-254 (Seq. ID Nos. 2211-2280).

TABLE 256
EBV Strain B95-8 EBNA1 HLA-DRB1*0301 (DR17)
Epitope Peptides

| SEQ ID NO. | Sequence |
|---|---|
| 2286 | FAEVLKDAIKDLVMT |
| 2287 | FENIAEGLRALLARS |
| 2288 | QCRLTPLSRLPFGMA |
| 2289 | RPFFHPVGEADYFEY |
| 2290 | GVFVYGGSKTSLYNL |

In some embodiments, the donor cell source is HLA-DRB1*0401, and the EBNA1 targeted T-cell subpopulation is primed and expanded with one or more EBNA1-derived peptides selected from Table 257 (Seq. ID. Nos. 2291-2295). In some embodiments, the donor cell source is HLA-DRB1*0401, and the EBNA1 targeted T-cell subpopulation is primed and expanded with EBNA1-derived peptides selected from Table 257 (Seq. ID. Nos. 2291-2295). In some embodiments, the donor cell source is HLA-DRB1*0401, and the EBNA1 targeted T-cell subpopulation is primed and expanded with EBNA1-derived peptides comprising the peptides of Table 257 (Seq. ID. Nos. 2291-2295). In some embodiments, the donor cell source is HLA-DRB1*0401, and the EBNA1 targeted T-cell subpopulation is primed and expanded with EBNA1-derived peptides comprising the peptides of Table 257 (Seq. ID. Nos. 2291-2295) and at least one additional set of peptides based on the donor cell source HLA-DR profile, wherein the at least one additional set of peptides are selected from the peptides of Tables 255-256 and 258-260. In some embodiments, the EBNA1-derived peptides also include one or more sets of HLA-A and HLA-B restricted peptides selected from Tables 241-254 (Seq. ID Nos. 2211-2280).

TABLE 257
EBV Strain B95-8 EBNA1 HLA-DRB1*0401 (DR4Dw4)
Epitope Peptides

| SEQ ID NO. | Sequence |
|---|---|
| 2291 | VCYFMVFLQTHIFAE |
| 2292 | KTSLYNLRRGTALAI |
| 2293 | AGVFVYGGSKTSLYN |
| 2294 | THIFAEVLKDAIKDL |
| 2295 | FENIAEGLRALLARS |

In some embodiments, the donor cell source is HLA-DRB1*0701, and the EBNA1 targeted T-cell subpopulation is primed and expanded with one or more EBNA1-derived peptides selected from Table 258 (Seq. ID. Nos. 2296-2300). In some embodiments, the donor cell source is HLA-DRB 1*0701, and the EBNA1 targeted T-cell subpopulation is primed and expanded with EBNA1-derived peptides selected from Table 258 (Seq. ID. Nos. 2296-2300). In some embodiments, the donor cell source is HLA-DRB1*0701, and the EBNA1 targeted T-cell subpopulation is primed and expanded with EBNA1-derived peptides comprising the peptides of Table 258 (Seq. ID. Nos. 2296-2300). In some embodiments, the donor cell source is HLA-DRB1*0701, and the EBNA1 targeted T-cell subpopulation is primed and expanded with EBNA1-derived peptides comprising the peptides of Table 258 (Seq. ID. Nos. 2296-2300) and at least one additional set of peptides based on the donor cell source HLA-DR profile, wherein the at least one additional set of peptides are selected from the peptides of Tables 255-257 and 259-260. In some embodiments, the EBNA1-derived peptides also include one or more sets of HLA-A and HLA-B restricted peptides selected from Tables 241-254 (Seq. ID Nos. 2211-2280).

TABLE 258
EBV Strain B95-8 EBNA1 HLA-DRB1*0701
Epitope Peptides

| SEQ ID NO. | Sequence |
|---|---|
| 2296 | VFVYGGSKTSLYNLR |
| 2297 | RPFFHPVGEADYFEY |
| 2298 | NPKFENIAEGLRALL |
| 2299 | RSHVERTTDEGTWVA |
| 2300 | CYFMVFLQTHIFAEV |

In some embodiments, the donor cell source is HLA-DRB1*1101, and the EBNA1 targeted T-cell subpopulation is primed and expanded with one or more EBNA1-derived peptides selected from Table 259 (Seq. ID. Nos. 2301-2305). In some embodiments, the donor cell source is HLA-DRB1*1101, and the EBNA1 targeted T-cell subpopulation is primed and expanded with EBNA1-derived peptides selected from Table 259 (Seq. ID. Nos. 2301-2305). In some embodiments, the donor cell source is HLA-DRB1*1101, and the EBNA1 targeted T-cell subpopulation is primed and expanded with EBNA1-derived peptides comprising the peptides of Table 259 (Seq. ID. 2301-2305). In some embodiments, the donor cell source is HLA-DRB1*1101, and the EBNA1 targeted T-cell subpopulation is primed and expanded with EBNA1-derived peptides comprising the peptides of Table 259 (Seq. ID. Nos. 2301-2305) and at least one additional set of peptides based on the donor cell source HLA-DR profile, wherein the at least one additional set of peptides are selected from the peptides of Tables 255-258 and 260. In some embodiments, the EBNA1-derived peptides also include one or more sets of HLA-A and HLA-B restricted peptides selected from Tables 241-254 (Seq. ID Nos. 2211-2280).

TABLE 259
EBV Strain B95-8 EBNA1 HLA-DRB1*1101
Epitope Peptides

| SEQ ID NO. | Sequence |
|---|---|
| 2301 | KTSLYNLRRGTALAI |
| 2302 | KGGWFGKHRGQGGSN |
| 2303 | THIFAEVLKDAIKDL |
| 2304 | PPWFPPMVEGAAAEG |
| 2305 | QCRLTPLSRLPFGMA |

In some embodiments, the donor cell source is HLA-DRB1*1501, and the EBNA1 targeted T-cell subpopulation is primed and expanded with one or more EBNA1-derived peptides selected from Table 260 (Seq. ID. Nos. 2306-2310). In some embodiments, the donor cell source is HLA-DRB1*1501, and the EBNA1 targeted T-cell subpopulation is primed and expanded with EBNA1-derived peptides selected from Table 260 (Seq. ID. Nos. 2306-2310). In some embodiments, the donor cell source is HLA-DRB1*1501, and the EBNA1 targeted T-cell subpopulation is primed and expanded with EBNA1-derived peptides comprising the peptides of Table 260 (Seq. ID. Nos. 2306-2310). In some embodiments, the donor cell source is HLA-DRB1*1501, and the EBNA1 targeted T-cell subpopulation is primed and expanded with EBNA1-derived peptides comprising the peptides of Table 260 (Seq. ID. Nos. 2306-2310) and at least one additional set of peptides based on the donor cell source HLA-DR profile, wherein the at least one additional set of peptides are selected from the peptides of Tables 255-259. In some embodiments, the EBNA1-derived peptides also include one or more sets of HLA-A and HLA-B restricted peptides selected from Tables 241-254 (Seq. ID Nos. 2211-2280).

TABLE 260
EBV Strain B95-8 EBNA1 HLA-DRB1*1501 (DR2b)
Epitope Peptides

| SEQ ID NO. | Sequence |
|---|---|
| 2306 | RESIVCYFMVFLQTH |
| 2307 | ESIVCYFMVFLQTHI |
| 2308 | VAGVFVYGGSKTSLY |
| 2309 | GVDLPPWFPPMVEGA |
| 2310 | LYNLRRGTALAIPQC |

Epstein-Barr Virus (EBV) Strain B95-8 EBNA2 Antigenic Peptides

In some embodiments, the MUSTANG composition includes Epstein-Barr Virus (EBV) Strain B95-8 EBNA2 specific T-cells. EBNA2 specific T-cells can be generated as described below using one or more antigenic peptides to EBNA2. In some embodiments, the EBNA2 specific T-cells are generated using one or more antigenic peptides to EBNA2, or a modified or heteroclitic peptide derived from a EBNA2 peptide. In some embodiments, EBNA2 specific T-cells are generated using a EBNA2 antigen library comprising a pool of peptides (for example 15mers) containing amino acid overlap (for example 11 amino acids of overlap) between each sequence formed by scanning the protein amino acid sequence SEQ. ID. No. 2311 (UniProt KB-P03211) for EBV Strain B95-8 EBNA2:

MPTFYLALHGGQTYHLIVDTDSLGNPSLSVIPSNPYQEQLSDTPLIPLT
IFVGENTGVPPPLPPPPPPPPPPPPPPPPPPPPPPPPPPSPPPPPPPPP
PPQRRDAWTQEPSPLDRDPLGYDVGHGPLASAMRMLWMANYIVRQSRGD
RGLILPQGPQTAPQARLVQPHVPPLRPTAPTILSPLSQPRLTPPQPLMM
PPRPTPPTPLPPATLTVPPRPTRPTTLPPTPLLTVLQRPTELQPTPSPP
RMHLPVLHVPDQSMEIPLTHQSTPNDPDSPEPRSPTVFYNIPPMPLPPS
QLPPPAAPAQPPPGVINDQQLHHLPSGPPWWPPICDPPQPSKTQGQSRG
QSRGRGRGRGRGRGKGKSRDKQRKPGGPWRPEPNTSSPSMPELSPVLGL
HQGQGAGDSPTPGPSNAAPVCRNSHTATPNVSPIHEPESHNSPEAPILF
PDDWYPPSIDPADLDESWDYIFETTESPSSDEDYVEGPSKRPRPSIQ.

In some embodiments, the EBNA2 specific T-cells are generated using one or more antigenic peptides to EBNA2, or a modified or heteroclitic peptide derived from a EBNA2 peptide. In some embodiments, the EBNA2 specific T-cells are generated with peptides that recognize class I MHC molecules. In some embodiments, the EBNA2 specific T-cells are generated with peptides that recognize class II MHC molecules. In some embodiments, the EBNA2 specific T-cells are generated with peptides that recognize both class I and class II MHC molecules.

In some embodiments, the EBNA2 peptides used to prime and expand a T-cell subpopulation includes specifically selected HLA-restricted peptides generated by determining the HLA profile of the donor source, and including peptides derived from EBNA2 that best match the donor's HLA. In some embodiments, the EBNA2 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting EBNA2 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 261-267, the HLA-B peptides are selected from the peptides of Tables 268-274, and the HLA-DR peptides are selected from the peptides of Tables 275-280. For example, if the donor cell source has an HLA profile that is HLA-A*01/*02:01; HLA-B*15:01/*18; and HLA-DRB1*0101/*0301, then the EBNA2 peptides used to prime and expand the EBNA2 specific T-cell subpopulation are restricted to the specific HLA profile, and may include the peptides identified in Table 261 (Seq. ID. Nos. 2312-2316) for HLA-A*01; Table 262 (Seq. ID. Nos. 2317-2321) for HLA-A*02:01; Table 270 (Seq. ID. Nos. 2357-2361) for HLA-B*15:01; Table 271 (Seq. ID. Nos. 2362-2366) for HLA-B*18; Table 275 (Seq. ID. Nos. 2382-2386) for HLA-DRB1*0101; and Table 276 (Seq. ID. Nos. 2387-2391) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the EBNA2 targeted T-cell subpopulation is primed and expanded with one or more EBNA2-derived peptides selected from Table 261 (Seq. ID. Nos. 2312-2316). In some embodiments, the donor cell source is HLA-A*01, and the EBNA2 targeted T-cell subpopulation is primed and expanded with EBNA2-derived peptides selected from Table 261 (Seq. ID. Nos. 2312-2316). In some embodiments, the donor cell source is HLA-A*01, and the EBNA2 targeted T-cell subpopulation is primed and expanded with EBNA2-derived peptides comprising the peptides of from Table 261 (Seq. ID. Nos. 2312-2316). In some embodiments, the donor cell source is HLA-A*01, and the EBNA2 targeted T-cell subpopulation is primed and expanded with EBNA2-derived peptides comprising the peptides of from Table 261 (Seq. ID. Nos. 2312-2316) and at least one additional set of peptides based on the donor cell source HLA-A profile, wherein the at least one additional set of peptides are selected from the peptides of Tables 262-267. In some embodiments, the EBNA2-derived peptides also include one or more sets of HLA-B and HLA-DR restricted peptides selected from Tables 268-280 (Seq. ID Nos. 2347-2411).

TABLE 261
EBV Strain B95-8 EBNA2 HLA-A*01 Epitope Peptides

| SEQ ID NO. | Sequence |
|---|---|
| 2312 | PLDRDPLGY |
| 2313 | PADLDESWDY |
| 2314 | TTESPSSDEDY |
| 2315 | SPEPRSPTVFY |
| 2316 | PSPLDRDPLGY |

In some embodiments, the donor cell source is HLA-A*02:01, and the EBNA2 targeted T-cell subpopulation is primed and expanded with one or more EBNA2-derived peptides selected from Table 262 (Seq. ID. Nos. 2317-2321). In some embodiments, the donor cell source is HLA-A*02:01, and the EBNA2 targeted T-cell subpopulation is primed and expanded with EBNA2-derived peptides selected from Table 262 (Seq. ID. Nos. 2317-2321). In some embodiments, the donor cell source is HLA-A*02:01, and the EBNA2 targeted T-cell subpopulation is primed and expanded with EBNA2-derived peptides comprising the peptides of Table 262 (Seq. ID. Nos. 2317-2321). In some embodiments, the donor cell source is HLA-A*02:01, and the EBNA2 targeted T-cell subpopulation is primed and expanded with EBNA2-derived peptides comprising the peptides of Table 262 (Seq. ID. Nos. 2317-2321) and at least one additional set of peptides based on the donor cell source HLA-A profile, wherein the at least one additional set of peptides are selected from the peptides of Tables 261, and 263-267. In some embodiments, the EBNA2-derived peptides also include one or more sets of HLA-B and HLA-DR restricted peptides selected from Tables 268-280 (Seq. ID Nos. 2347-2411).

TABLE 262
EBV Strain B95-8 EBNA2 HLA-A*02:01
Epitope Peptides

| SEQ ID NO. | Sequence |
|---|---|
| 2317 | HLIVDTDSL |
| 2318 | SLGNPSLSV |
| 2319 | TLPPTPLLTV |
| 2320 | VINDQQLHHL |
| 2321 | ALHGGQTYHL |

In some embodiments, the donor cell source is HLA-A*03, and the EBNA2 targeted T-cell subpopulation is primed and expanded with one or more EBNA2-derived peptides selected from Table 263 (Seq. ID. Nos. 2322-2326). In some embodiments, the donor cell source is HLA-A*03, and the EBNA2 targeted T-cell subpopulation is primed and expanded with EBNA2-derived peptides selected from Table 263 (Seq. ID. Nos. 2322-2326). In some embodiments, the donor cell source is HLA-A*03, and the EBNA2 targeted T-cell subpopulation is primed and expanded with EBNA2-derived peptides comprising the peptides of Table 263 (Seq. ID. 2322-2326). In some embodiments, the donor cell source is HLA-A*03, and the EBNA2 targeted T-cell subpopulation is primed and expanded with EBNA2-derived peptides comprising the peptides of Table 263 (Seq. ID. Nos. 2322-2326) and at least one additional set of peptides based on the donor cell source HLA-A profile, wherein the at least one additional set of peptides are selected from the peptides of Tables 261-262 and 264-267. In some embodiments, the EBNA2-derived peptides also include one or more sets of HLA-B and HLA-DR restricted peptides selected from Tables 268-280 (Seq. ID Nos. 2347-2411).

TABLE 263
EBV Strain B95-8 EBNA2 HLA-A*03 Epitope Peptides

| SEQ ID NO. | Sequence |
|---|---|
| 2322 | RGRGRGRGK |
| 2323 | PLDRDPLGY |
| 2324 | YLALHGGQTY |
| 2325 | RLTPPQPLMM |
| 2326 | SVIPSNPYQE |

In some embodiments, the donor cell source is HLA-A*11:01, and the EBNA2 targeted T-cell subpopulation is primed and expanded with one or more EBNA2-derived peptides selected from Table 264 (Seq. ID. Nos. 2327-2331). In some embodiments, the donor cell source is HLA-A*11:01, and the EBNA2 targeted T-cell subpopulation is primed and expanded with EBNA2-derived peptides selected from Table 264 (Seq. ID. Nos. 2327-2331). In some embodiments, the donor cell source is HLA-A*11:01, and the EBNA2 targeted T-cell subpopulation is primed and expanded with EBNA2-derived peptides comprising the peptides of Table 264 (Seq. ID. Nos. 2327-2331). In some embodiments, the donor cell source is HLA-A*11:01, and the EBNA2 targeted T-cell subpopulation is primed and expanded with EBNA2-derived peptides comprising the peptides of Table 264 (Seq. ID. Nos. 2327-2331), and at least one additional set of peptides based on the donor cell source HLA-A profile, wherein the at least one additional set of peptides are selected from the peptides of Tables 261-263 and 265-267. In some embodiments, the EBNA2-derived peptides also include one or more sets of HLA-B and HLA-DR restricted peptides selected from Tables 268-280 (Seq. ID Nos. 2347-2411).

TABLE 264
EBV Strain B95-8 EBNA2 HLA-A*11:01
Epitope Peptides

| SEQ ID NO. | Sequence |
|---|---|
| 2327 | PSNAAPVCR |
| 2328 | WTQEPSPLDR |
| 2329 | PTPLLTVLQR |
| 2330 | LVQPHVPPLR |
| 2331 | MLWMANYIVR |

In some embodiments, the donor cell source is HLA-A*24:02, and the EBNA2 targeted T-cell subpopulation is primed and expanded with one or more EBNA2-derived peptides selected from Table 265 (Seq. ID. Nos. 2332-2336). In some embodiments, the donor cell source is HLA-A*24:02, and the EBNA2 targeted T-cell subpopulation is primed and expanded with EBNA2-derived peptides selected from Table 265 (Seq. ID. Nos. 2332-2336). In some embodiments, the donor cell source is HLA-A*24:02, and the EBNA2 targeted T-cell subpopulation is primed and expanded with EBNA2-derived peptides comprising the peptides of Table 265 (Seq. ID. Nos. 2332-2336). In some embodiments, the donor cell source is HLA-A*24:02, and the EBNA2 targeted T-cell subpopulation is primed and expanded with EBNA2-derived peptides comprising the peptides of Table 265 (Seq. ID. Nos. 2332-2336), and at least one additional set of peptides based on the donor cell source HLA-A profile, wherein the at least one additional set of peptides are selected from the peptides of Tables 261-264 and 266-267. In some embodiments, the EBNA2-derived peptides also include one or more sets of HLA-B and HLA-DR restricted peptides selected from Tables 268-280 (Seq. ID Nos. 2347-2411).

TABLE 265
EBV Strain B95-8 EBNA2 HLA-A*24:02
Epitope Peptides

| SEQ ID NO. | Sequence |
|---|---|
| 2332 | GYDVGHGPL |
| 2333 | FYNIPPMPL |
| 2334 | NSPEAPILF |
| 2335 | TTLPPTPLL |
| 2336 | TVLQRPTEL |

In some embodiments, the donor cell source is HLA-A*26, and the EBNA2 targeted T-cell subpopulation is primed and expanded with one or more EBNA2-derived peptides selected from Table 266 (Seq. ID. Nos. 2337-2341). In some embodiments, the donor cell source is HLA-A*26, and the EBNA2 targeted T-cell subpopulation is primed and expanded with EBNA2-derived peptides selected from Table 266 (Seq. ID. Nos. 2337-2341). In some embodiments, the donor cell source is HLA-A*26, and the EBNA2 targeted T-cell subpopulation is primed and expanded with EBNA2-derived peptides comprising the peptides of Table 266 (Seq. ID. Nos. 2337-2341). In some embodiments, the donor cell source is HLA-A*26, and the EBNA2 targeted T-cell subpopulation is primed and expanded with EBNA2-derived peptides comprising the peptides of Table 266 (Seq. ID. Nos. 2337-2341) and at least one additional set of peptides based on the donor cell source HLA-A profile, wherein the at least one additional set of peptides are selected from the peptides of Tables 261-265 and 267. In some embodiments, the EBNA2-derived peptides also include one or more sets of HLA-B and HLA-DR restricted peptides selected from Tables 268-280 (Seq. ID Nos. 2347-2411).

TABLE 266
EBV Strain B95-8 EBNA2 HLA-A*26 Epitope Peptides

| SEQ ID NO. | Sequence |
|---|---|
| 2337 | LVQPHVPPL |
| 2338 | ETTESPSSD |
| 2339 | DTPLIPLTIF |
| 2340 | DTDSLGNPSL |
| 2341 | DVGHGPLASA |

In some embodiments, the donor cell source is HLA-A*68:01, and the EBNA2 targeted T-cell subpopulation is primed and expanded with one or more EBNA2-derived peptides selected from Table 267 (Seq. ID. Nos. 2342-2346). In some embodiments, the donor cell source is HLA-A*68:01, and the EBNA2 targeted T-cell subpopulation is primed and expanded with EBNA2-derived peptides selected from Table 267 (Seq. ID. Nos. 2342-2346). In some embodiments, the donor cell source is HLA-A*68:01, and the EBNA2 targeted T-cell subpopulation is primed and expanded with EBNA2-derived peptides comprising the peptides of Table 267 (Seq. ID. Nos. 2342-2346). In some embodiments, the donor cell source is HLA-A*68:01, and the EBNA2 targeted T-cell subpopulation is primed and expanded with EBNA2-derived peptides comprising the peptides of Table 267 (Seq. ID. Nos. 2342-2346), and at least one additional set of peptides based on the donor cell source HLA-A profile, wherein the at least one additional set of peptides are selected from the peptides of Tables 261-266. In some embodiments, the EBNA2-derived peptides also include one or more sets of HLA-B and HLA-DR restricted peptides selected from Tables 268-280 (Seq. ID Nos. 2347-2411).

TABLE 267
EBV Strain B95-8 EBNA2 HLA-A*68:01
Epitope Peptides

| SEQ ID NO. | Sequence |
|---|---|
| 2342 | PTILSPLSQPR |
| 2343 | PATLTVPPR |
| 2344 | PTPLLTVLQR |
| 2345 | MLWMANYIVR |
| 2346 | LVQPHVPPLR |

In some embodiments, the donor cell source is HLA-B*07:02, and the EBNA2 targeted T-cell subpopulation is primed and expanded with one or more EBNA2-derived peptides selected from Table 268 (Seq. ID. Nos. 2347-2351). In some embodiments, the donor cell source is HLA-B*07:02, and the EBNA2 targeted T-cell subpopulation is primed and expanded with EBNA2-derived peptides selected from Table 268 (Seq. ID. Nos. 2347-2351). In some embodiments, the donor cell source is HLA-B*07:02, and the EBNA2 targeted T-cell subpopulation is primed and expanded with EBNA2-derived peptides comprising the peptides of Table 268 (Seq. ID. Nos. 2347-2351). In some embodiments, the donor cell source is HLA-B*07:02, and the EBNA2 targeted T-cell subpopulation is primed and expanded with EBNA2-derived peptides comprising the peptides of Table 268 (Seq. ID. Nos. 2347-2351), and at least one additional set of peptides based on the donor cell source HLA-B profile, wherein the at least one additional set of peptides are selected from the peptides of Tables 269-274. In some embodiments, the EBNA2-derived peptides also include one or more sets of HLA-A and HLA-DR restricted peptides selected from Tables 261-267 and 275-280 (Seq. ID Nos. 2312-2346 and 2382-2411).

TABLE 268
EBV Strain B95-8 EBNA2 HLA-B*07:02
Epitope Peptides

| SEQ ID NO. | Sequence |
|---|---|
| 2347 | TPSPPRMHL |
| 2348 | PPTPLLTVL |
| 2349 | EPSPLDRDPL |
| 2350 | PPRPTPPTPL |
| 2351 | PPRPTRPTTL |

In some embodiments, the donor cell source is HLA-B*08, and the EBNA2 targeted T-cell subpopulation is primed and expanded with one or more EBNA2-derived peptides selected from Table 269 (Seq. ID. Nos. 2352-2356). In some embodiments, the donor cell source is HLA-B*08, and the EBNA2 targeted T-cell subpopulation is primed and expanded with EBNA2-derived peptides selected from Table 269 (Seq. ID. Nos. 2352-2356). In some embodiments, the donor cell source is HLA-B*08, and the EBNA2 targeted T-cell subpopulation is primed and expanded with EBNA2-derived peptides comprising the peptides of Table 269 (Seq. ID. Nos. 2352-2356). In some embodiments, the donor cell source is HLA-B*08, and the EBNA2 targeted T-cell subpopulation is primed and expanded with EBNA2-derived peptides comprising the peptides of Table 269 (Seq. ID. Nos. 2352-2356) and at least one additional set of peptides based on the donor cell source HLA-B profile, wherein the at least one additional set of peptides are selected from the peptides of Tables 268 and 270-274. In some embodiments, the EBNA2-derived peptides also include one or more sets of HLA-A and HLA-DR restricted peptides selected from Tables 241-261-267 and 275-280 (Seq. ID Nos. 2312-2346 and 2382-2411).

TABLE 269
EBV Strain B95-8 EBNA2 HLA-B*08 Epitope Peptides

| SEQ ID NO. | Sequence |
|---|---|
| 2352 | SPLDRDPL |
| 2353 | SKRPRPSI |
| 2354 | PPRMHLPVL |
| 2355 | SRGDRGLIL |
| 2356 | RGKGKSRDK |

In some embodiments, the donor cell source is HLA-B*15:01, and the EBNA2 targeted T-cell subpopulation is primed and expanded with one or more EBNA2-derived peptides selected from Table 270 (Seq. ID. Nos. 2357-2361). In some embodiments, the donor cell source is HLA-B*15:01, and the EBNA2 targeted T-cell subpopulation is primed and expanded with EBNA2-derived peptides selected from Table 270 (Seq. ID. Nos. 2357-2361). In some embodiments, the donor cell source is HLA-B*15:01, and the EBNA2 targeted T-cell subpopulation is primed and expanded with EBNA2-derived peptides comprising the peptides of Table 270 (Seq. ID. Nos. 2357-2361). In some embodiments, the donor cell source is HLA-B*15:01, and the EBNA2 targeted T-cell subpopulation is primed and expanded with EBNA2-derived peptides comprising the peptides of Table 270 (Seq. ID. Nos. 2357-2361) and at least one additional set of peptides based on the donor cell source HLA-B profile, wherein the at least one additional set of peptides are selected from the peptides of Tables 268-269 and 271-274. In some embodiments, the EBNA2-derived peptides also include one or more sets of HLA-A and HLA-DR restricted peptides selected from Tables 261-267 and 275-280 (Seq. ID Nos. 2312-2346 and 2382-2411).

TABLE 270
EBV Strain B95-8 EBNA2 HLA-B*15:01 (B62)
Epitope Peptides

| SEQ ID NO. | Sequence |
|---|---|
| 2357 | PLDRDPLGY |
| 2358 | YLALHGGQTY |
| 2359 | SLSVIPSNPY |
| 2360 | DLDESWDYIF |
| 2361 | PLPPATLTVP |

In some embodiments, the donor cell source is HLA-B*18, and the EBNA2 targeted T-cell subpopulation is primed and expanded with one or more EBNA2-derived peptides selected from Table 271 (Seq. ID. Nos. 2362-2366). In some embodiments, the donor cell source is HLA-B*18, and the EBNA2 targeted T-cell subpopulation is primed and expanded with EBNA2-derived peptides selected from Table 271 (Seq. ID. Nos. 2362-2366). In some embodiments, the donor cell source is HLA-B*18, and the EBNA2 targeted T-cell subpopulation is primed and expanded with EBNA2-derived peptides comprising the peptides of Table 271 (Seq. ID. Nos. 2362-2366). In some embodiments, the donor cell source is HLA-B*18, and the EBNA2 targeted T-cell subpopulation is primed and expanded with EBNA2-derived peptides comprising the peptides of Table 271 (Seq. ID. Nos. 2362-2366) and at least one additional set of peptides based on the donor cell source HLA-B profile, wherein the at least one additional set of peptides are selected from the peptides of Tables 268-270 and 272-274. In some embodiments, the EBNA2-derived peptides also include one or more sets of HLA-A and HLA-DR restricted peptides selected from Tables 261-267 and 275-280 (Seq. ID Nos. 2312-2346 and 2382-2411).

TABLE 271
EBV Strain B95-8 EBNA2 HLA-B*18 Epitope Peptides

| SEQ ID NO. | Sequence |
|---|---|
| 2362 | PEPRSPTVF |
| 2363 | DESWDYIF |
| 2364 | QEQLSDTPL |
| 2365 | PELSPVLGL |
| 2366 | DEDYVEGP |

In some embodiments, the donor cell source is HLA-B*27:05, and the EBNA2 targeted T-cell subpopulation is primed and expanded with one or more EBNA2-derived peptides selected from Table 272 (Seq. ID. Nos. 2367-2371). In some embodiments, the donor cell source is HLA-B*27:05, and the EBNA2 targeted T-cell subpopulation is primed and expanded with EBNA2-derived peptides selected from Table 272 (Seq. ID. Nos. 2367-2371). In some embodiments, the donor cell source is HLA-B*27:05, and the EBNA2 targeted T-cell subpopulation is primed and expanded with EBNA2-derived peptides comprising the peptides of Table 272 (Seq. ID. Nos. 2367-2371). In some embodiments, the donor cell source is HLA-B*27:05, and the EBNA2 targeted T-cell subpopulation is primed and expanded with EBNA2-derived peptides comprising the peptides of Table 272 (Seq. ID. Nos. 2367-2371) and at least one additional set of peptides based on the donor cell source HLA-B profile, wherein the at least one additional set of peptides are selected from the peptides of Tables 268-271 and 273-274. In some embodiments, the EBNA2-derived peptides also include one or more sets of HLA-A and HLA-DR restricted peptides selected from 261-267 and 275-280 (Seq. ID Nos. 2312-2346 and 2382-2411).

TABLE 272
EBV Strain B95-8 EBNA2 HLA-B*27:05
Epitope Peptides

| SEQ ID NO. | Sequence |
|---|---|
| 2367 | GRGKGKSRDK |
| 2368 | PRLTPPQPLM |
| 2369 | QRKPGGPWR |
| 2370 | PRPTPPTPL |
| 2371 | PRPTRPTTL |

In some embodiments, the donor cell source is HLA-B*35:01, and the EBNA2 targeted T-cell subpopulation is primed and expanded with one or more EBNA2-derived peptides selected from Table 273 (Seq. ID. Nos. 2372-2376). In some embodiments, the donor cell source is HLA-B*35:01, and the EBNA2 targeted T-cell subpopulation is primed and expanded with EBNA2-derived peptides selected from Table 273 (Seq. ID. Nos. 2372-2376). In some embodiments, the donor cell source is HLA-B*35:01, and the EBNA2 targeted T-cell subpopulation is primed and expanded with EBNA2-derived peptides comprising the peptides of Table 273 (Seq. ID. Nos. 2372-2376). In some embodiments, the donor cell source is HLA-B*35:01, and the EBNA2 targeted T-cell subpopulation is primed and expanded with EBNA2-derived peptides comprising the peptides of Table 273 (Seq. ID. Nos. 2372-2376) and at least one additional set of peptides based on the donor cell source HLA-B profile, wherein the at least one additional set of peptides are selected from the peptides of Tables 268-272 and 274. In some embodiments, the EBNA2-derived peptides also include one or more sets of HLA-A and HLA-DR restricted peptides selected from 261-267 and 275-280 (Seq. ID Nos. 2312-2346 and 2382-2411).

TABLE 273
EBV Strain B95-8 EBNA2 HLA-B*35:01
Epitope Peptides

| SEQ ID NO. | Sequence |
|---|---|
| 2372 | EPRSPTVFY |
| 2373 | VPDQSMHPL |
| 2374 | LPPTPLLTVL |
| 2375 | SPPRMHLPVL |
| 2376 | SPLDRDPLGY |

In some embodiments, the donor cell source is HLA-B*58:02, and the EBNA2 targeted T-cell subpopulation is primed and expanded with one or more EBNA2-derived peptides selected from Table 274 (Seq. ID. Nos. 2377-2381). In some embodiments, the donor cell source is HLA-B*58:02, and the EBNA2 targeted T-cell subpopulation is primed and expanded with EBNA2-derived peptides selected from Table 274 (Seq. ID. Nos. 2377-2381). In some embodiments, the donor cell source is HLA-B*58:02, and the EBNA2 targeted T-cell subpopulation is primed and expanded with EBNA2-derived peptides comprising the peptides of Table 274 (Seq. ID. Nos. 2377-2381). In some embodiments, the donor cell source is HLA-B*58:02, and the EBNA2 targeted T-cell subpopulation is primed and expanded with EBNA2-derived peptides comprising the peptides of Table 274 (Seq. ID. Nos. 2377-2381) and at least one additional set of peptides based on the donor cell source HLA-B profile, wherein the at least one additional set of peptides are selected from the peptides of Tables 268-273. In some embodiments, the EBNA2-derived peptides also include one or more sets of HLA-A and HLA-DR restricted peptides selected from Tables 261-267 and 275-280 (Seq. ID Nos. 2312-2346 and 2382-2411).

TABLE 274
EBV Strain B95-8 EBNA2 HLA-B*58:02
Epitope Peptides

| SEQ ID NO. | Sequence |
|---|---|
| 2377 | RSPTVFYNI |
| 2378 | NSPEAPILF |
| 2379 | LALHGGQTY |
| 2380 | PSGPPWWPPI |
| 2381 | PSMPELSPVL |

In some embodiments, the donor cell source is HLA-DRB1*0101, and the EBNA2 targeted T-cell subpopulation is primed and expanded with one or more EBNA2-derived peptides selected from Table 275 (Seq. ID. Nos. 2382-2386). In some embodiments, the donor cell source is HLA-DRB1*0101, and the EBNA2 targeted T-cell subpopulation is primed and expanded with EBNA2-derived peptides selected from Table 275 (Seq. ID. Nos. 2382-2386). In some embodiments, the donor cell source is HLA-DRB1*0101, and the EBNA2 targeted T-cell subpopulation is primed and expanded with EBNA2-derived peptides comprising the peptides of Table 275 (Seq. ID. Nos. 2382-2386). In some embodiments, the donor cell source is HLA-DRB1*0101, and the EBNA2 targeted T-cell subpopulation is primed and expanded with EBNA2-derived peptides comprising the peptides of Table 275 (Seq. ID. Nos. 2382-2386) and at least one additional set of peptides based on the donor cell source HLA-DR profile, wherein the at least one additional set of peptides are selected from the peptides of Tables 276-280. In some embodiments, the EBNA2-derived peptides also include one or more sets of HLA-A and HLA-B restricted peptides selected from Tables 261-274 (Seq. ID Nos. 2312-2381).

TABLE 275
EBV Strain B95-8 EBNA2 HLA-DRB1*0101
Epitope Peptides

| SEQ ID NO. | Sequence |
|---|---|
| 2382 | QPHVPPLRPTAPTIL |
| 2383 | SNPYQEQLSDTPLIP |
| 2384 | PTVFYNIPPMPLPPS |
| 2385 | DQQLHHLPSGPPWWP |
| 2386 | RGLILPQGPQTAPQA |

In some embodiments, the donor cell source is HLA-DRB1*0301, and the EBNA2 targeted T-cell subpopulation is primed and expanded with one or more EBNA2-derived peptides selected from Table 276 (Seq. ID. Nos. 2387-2391). In some embodiments, the donor cell source is HLA-DRB1*0301, and the EBNA2 targeted T-cell subpopulation is primed and expanded with EBNA2-derived peptides selected from Table 276 (Seq. ID. Nos. 2387-2391). In some embodiments, the donor cell source is HLA-DRB1*0301, and the EBNA2 targeted T-cell subpopulation is primed and expanded with EBNA2-derived peptides comprising the peptides of Table 276 (Seq. ID. Nos. 2387-2391). In some embodiments, the donor cell source is HLA-DRB1*0301, and the EBNA2 targeted T-cell subpopulation is primed and expanded with EBNA2-derived peptides comprising the peptides of Table 276 (Seq. ID. Nos. 2387-2391) and at least one additional set of peptides based on the donor cell source HLA-DR profile, wherein the at least one additional set of peptides are selected from the peptides of Tables 275 and 277-280. In some embodiments, the EBNA2-derived peptides also include one or more sets of HLA-A and HLA-B restricted peptides selected from Tables 261-274 (Seq. ID Nos. 2312-2381).

TABLE 276
EBV Strain B95-8 EBNA2 HLA-DRB1*0301 (DR17)
Epitope Peptides

| SEQ ID NO. | Sequence |
|---|---|
| 2387 | PPGVINDQQLHHLPS |
| 2388 | PTILSPLSQPRLTPP |
| 2389 | TYHLIVDTDSLGNPS |
| 2390 | HLIVDTDSLGNPSLS |
| 2391 | ASAMRMLWMANYIVR |

In some embodiments, the donor cell source is HLA-DRB1*0401, and the EBNA2 targeted T-cell subpopulation is primed and expanded with one or more EBNA2-derived peptides selected from Table 277 (Seq. ID. Nos. 2392-2396). In some embodiments, the donor cell source is HLA-DRB 1*0401, and the EBNA2 targeted T-cell subpopulation is primed and expanded with EBNA2-derived peptides selected from Table 277 (Seq. ID. Nos. 2392-2396). In some embodiments, the donor cell source is HLA-DRB1*0401, and the EBNA2 targeted T-cell subpopulation is primed and expanded with EBNA2-derived peptides comprising the peptides of Table 277 (Seq. ID. Nos. 2392-2396). In some embodiments, the donor cell source is HLA-DRB1*0401, and the EBNA2 targeted T-cell subpopulation is primed and expanded with EBNA2-derived peptides comprising the peptides of Table 277 (Seq. ID. Nos. 2392-2396) and at least one additional set of peptides based on the donor cell source HLA-DR profile, wherein the at least one additional set of peptides are selected from the peptides of Tables 275-276 and 278-280. In some embodiments, the EBNA2-derived peptides also include one or more sets of HLA-A and HLA-B restricted peptides selected from Tables 261-274 (Seq. ID Nos. 2312-2381).

TABLE 277
EBV Strain B95-8 EBNA2 HLA-DRB1*0401 (DR4Dw4)
Epitope Peptides

| SEQ ID NO. | Sequence |
|---|---|
| 2392 | MPTFYLALHGGQTYH |
| 2393 | GGPWRPEPNTSSPSM |
| 2394 | PTILSPLSQPRLTPP |
| 2395 | PTPLLTVLQRPTELQ |
| 2396 | DQSMHPLTHQSTPND |

In some embodiments, the donor cell source is HLA-DRB1*0701, and the EBNA2 targeted T-cell subpopulation is primed and expanded with one or more EBNA2-derived peptides selected from Table 278 (Seq. ID. Nos. 2397-2401). In some embodiments, the donor cell source is HLA-DRB1*0701, and the EBNA2 targeted T-cell subpopulation is primed and expanded with EBNA2-derived peptides selected from Table 278 (Seq. ID. Nos. 2397-2401). In some embodiments, the donor cell source is HLA-DRB1*0701, and the EBNA2 targeted T-cell subpopulation is primed and expanded with EBNA2-derived peptides comprising the peptides of Table 278 (Seq. ID. Nos. 2397-2401). In some embodiments, the donor cell source is HLA-DRB1*0701, and the EBNA2 targeted T-cell subpopulation is primed and expanded with EBNA2-derived peptides comprising the peptides of Table 278 (Seq. ID. Nos. 2397-2401) and at least one additional set of peptides based on the donor cell source HLA-DR profile, wherein the at least one additional set of peptides are selected from the peptides of Tables 275-277 and 279-280. In some embodiments, the EBNA2-derived peptides also include one or more sets of HLA-A and HLA-B restricted peptides selected from Tables 261-274 (Seq. ID Nos. 2312-2381).

TABLE 278
EBV Strain B95-8 EBNA2 HLA-DRB1*0701
Epitope Peptides

| SEQ ID NO. | Sequence |
|---|---|
| 2397 | NPSLSVIPSNPYQEQ |
| 2398 | PADLDESWDYIFETT |
| 2399 | DYIFETTESPSSDED |
| 2400 | YLALHGGQTYHLIVD |
| 2401 | YHLIVDTDSLGNPSL |

In some embodiments, the donor cell source is HLA-DRB1*1101, and the EBNA2 targeted T-cell subpopulation is primed and expanded with one or more EBNA2-derived peptides selected from Table 279 (Seq. ID. Nos. 2402-2406). In some embodiments, the donor cell source is HLA-DRB1*1101, and the EBNA2 targeted T-cell subpopulation is primed and expanded with EBNA2-derived peptides selected from Table 279 (Seq. ID. Nos. 2402-2406). In some embodiments, the donor cell source is HLA-DRB1*1101, and the EBNA2 targeted T-cell subpopulation is primed and expanded with EBNA2-derived peptides comprising the peptides of Table 279 (Seq. ID. 2402-2406). In some embodiments, the donor cell source is HLA-DRB1*1101, and the EBNA2 targeted T-cell subpopulation is primed and expanded with EBNA2-derived peptides comprising the peptides of Table 279 (Seq. ID. Nos. 2402-2406) and at least one additional set of peptides based on the donor cell source HLA-DR profile, wherein the at least one additional set of peptides are selected from the peptides of Tables 275-278 and 280. In some embodiments, the EBNA2-derived peptides also include one or more sets of HLA-A and HLA-B restricted peptides selected from Tables 261-274 (Seq. ID Nos. 2312-2381).

TABLE 279
EBV Strain B95-8 EBNA2 HLA-DRB1*1101
Epitope Peptides

| SEQ ID NO. | Sequence |
|---|---|
| 2402 | GQTYHLIVDTDSLGN |
| 2403 | TPLLTVLQRPTELQP |
| 2404 | PQPLMMPPRPTPPTP |
| 2405 | DQSMHPLTHQSTPND |
| 2406 | VINDQQLHHLPSGPP |

In some embodiments, the donor cell source is HLA-DRB1*1501, and the EBNA2 targeted T-cell subpopulation is primed and expanded with one or more EBNA2-derived peptides selected from Table 280 (Seq. ID. Nos. 2407-2411). In some embodiments, the donor cell source is HLA-DRB1*1501, and the EBNA2 targeted T-cell subpopulation is primed and expanded with EBNA2-derived peptides selected from Table 280 (Seq. ID. Nos. 2407-2411). In some embodiments, the donor cell source is HLA-DRB1*1501, and the EBNA2 targeted T-cell subpopulation is primed and expanded with EBNA2-derived peptides comprising the peptides of Table 280 (Seq. ID. Nos. 2407-2411). In some embodiments, the donor cell source is HLA-DRB1*1501, and the EBNA2 targeted T-cell subpopulation is primed and expanded with EBNA2-derived peptides comprising the peptides of Table 280 (Seq. ID. Nos. 2407-2411) and at least one additional set of peptides based on the donor cell source HLA-DR profile, wherein the at least one additional set of peptides are selected from the peptides of Tables 275-279. In some embodiments, the EBNA2-derived peptides also include one or more sets of HLA-A and HLA-B restricted peptides selected from Tables 261-274 (Seq. ID Nos. 2312-2381).

TABLE 280
EBV Strain B95-8 EBNA2 HLA-DRB1*1501
(DR2b) Epitope Peptides

| SEQ ID NO. | Sequence |
|---|---|
| 2407 | LIPLTIFVGENTGVP |
| 2408 | YHLIVDTDSLGNPSL |
| 2409 | TIFVGENTGVPPPLP |
| 2410 | MLWMANYIVRQSRGD |
| 2411 | QPRLTPPQPLMMPPR |

Human Papillomavirus (HPV) Strain 16 E6 Antigenic Peptides

In some embodiments, the MUSTANG composition includes Human Papillomavirus (HPV) Strain 16 E6 specific T-cells. E6 specific T-cells can be generated as described below using one or more antigenic peptides to E6. In some embodiments, the E6 specific T-cells are generated using one or more antigenic peptides to E6, or a modified or heteroclitic peptide derived from a E6 peptide. In some embodiments, E6 specific T-cells are generated using a E6 antigen library comprising a pool of peptides (for example 15mers) containing amino acid overlap (for example 11 amino acids of overlap) between each sequence formed by scanning the protein amino acid sequence SEQ. ID. No. 2412 (UniProt KB-P03126) for HPV Strain 16-8 E6:

MHQKRTAMFQDPQERPRKLPQLCTELQTTIHDIILECVYCKQQLLRREVY
DFAFRDLCIVYRDGNPYAVCDKCLKFYSKISEYRHYCYSLYGTTLEQQYN
KPLCDLLIRCINCQKPLCPEEKQRHLDKKQRFHNIRGRWTGRCMSCCRSS
RTRRETQL.

In some embodiments, the E6 specific T-cells are generated using one or more antigenic peptides to E6, or a modified or heteroclitic peptide derived from a E6 peptide. In some embodiments, the E6 specific T-cells are generated with peptides that recognize class I MHC molecules. In some embodiments, the E6 specific T-cells are generated with peptides that recognize class II MHC molecules. In some embodiments, the E6 specific T-cells are generated with peptides that recognize both class I and class II MHC molecules.

In some embodiments, the E6 peptides used to prime and expand a T-cell subpopulation includes specifically selected HLA-restricted peptides generated by determining the HLA profile of the donor source, and including peptides derived from E6 that best match the donor's HLA. In some embodiments, the E6 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting E6 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 281-287, the HLA-B peptides are selected from the peptides of Tables 288-294, and the HLA-DR peptides are selected from the peptides of Tables 295-280. For example, if the donor cell source has an HLA profile that is HLA-A*01/*02:01; HLA-B*15:01/*18; and HLA-DRB1*0101/*0301, then the E6 peptides used to prime and expand the E6 specific T-cell subpopulation are restricted to the specific HLA profile, and may include the peptides identified in Table 281 (Seq. ID. Nos. 2413-2417) for HLA-A*01; Table 282 (Seq. ID. Nos. 2418-2422) for HLA-A*02:01; Table 290 (Seq. ID. Nos. 2458-2462) for HLA-B*15:01; Table 291 (Seq. ID. Nos. 2463-2467) for HLA-B*18; Table 295 (Seq. ID. Nos. 2483-2487) for HLA-DRB1*0101; and Table 296 (Seq. ID. Nos. 2488-2492) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the E6 targeted T-cell subpopulation is primed and expanded with one or more E6-derived peptides selected from Table 281 (Seq. ID. Nos. 2413-2417). In some embodiments, the donor cell source is HLA-A*01, and the E6 targeted T-cell subpopulation is primed and expanded with E6-derived peptides selected from Table 281 (Seq. ID. Nos. 2413-2417). In some embodiments, the donor cell source is HLA-A*01, and the E6 targeted T-cell subpopulation is primed and expanded with E6-derived peptides comprising the peptides of from Table 281 (Seq. ID. Nos. 2413-2417). In some embodiments, the donor cell source is HLA-A*01, and the E6 targeted T-cell subpopulation is primed and expanded with E6-derived peptides comprising the peptides of from Table 281 (Seq. ID. Nos. 2413-2417) and at least one additional set of peptides based on the donor cell source HLA-A profile, wherein the at least one additional set of peptides are selected from the peptides of Tables 282-287. In some embodiments, the E6-derived peptides also include one or more sets of HLA-B and HLA-DR restricted peptides selected from Tables 288-300 (Seq. ID Nos. 2448-2512).

TABLE 281
HPV Strain 16 E6 HLA-A*01 Epitope
Peptides

| SEQ ID NO. | Sequence |
|---|---|
| 2413 | YAVCDKCLKFY |
| 2414 | SEYRHYCYSLY |
| 2415 | CKQQLLRREVY |
| 2416 | IHDIILECVY |
| 2417 | YSKISEYRHY |

In some embodiments, the donor cell source is HLA-A*02:01, and the E6 targeted T-cell subpopulation is primed and expanded with one or more E6-derived peptides selected from Table 282 (Seq. ID. Nos. 2418-2422). In some embodiments, the donor cell source is HLA-A*02:01, and the E6 targeted T-cell subpopulation is primed and expanded with MAGE-A3-derived peptides selected from Table 282 (Seq. ID. Nos. 2418-2422). In some embodiments, the donor cell source is HLA-A*02:01, and the E6 targeted T-cell subpopulation is primed and expanded with E6-derived peptides comprising the peptides of Table 282 (Seq. ID. Nos. 2418-2422). In some embodiments, the donor cell source is HLA-A*02:01, and the E6 targeted T-cell subpopulation is primed and expanded with E6-derived peptides comprising the peptides of Table 282 (Seq. ID. Nos. 2418-2422) and at least one additional set of peptides based on the donor cell source HLA-A profile, wherein the at least one additional set of peptides are selected from the peptides of Tables 281, and 283-287. In some embodiments, the E6-derived peptides also include one or more sets of HLA-B and HLA-DR restricted peptides selected from Tables 288-300 (Seq. ID Nos. 2448-2512).

TABLE 282
HPV Strain 16 E6 HLA-A*02:01 Epitope Peptides

| SEQ ID NO. | Sequence |
|---|---|
| 2418 | TIHDIILECV |
| 2419 | QLCIELQTTI |
| 2420 | PLCDLLIRCI |
| 2421 | KLPQLCTEL |
| 2422 | QLCTELQTT |

In some embodiments, the donor cell source is HLA-A*03, and the E6 targeted T-cell subpopulation is primed and expanded with one or more E6-derived peptides selected from Table 283 (Seq. ID. Nos. 2423-2427). In some embodiments, the donor cell source is HLA-A*03, and the E6 targeted T-cell subpopulation is primed and expanded with E6-derived peptides selected from Table 283 (Seq. ID. Nos. 2423-2427). In some embodiments, the donor cell source is HLA-A*03, and the E6 targeted T-cell subpopulation is primed and expanded with E6-derived peptides comprising the peptides of Table 283 (Seq. ID. 2423-2427). In some embodiments, the donor cell source is HLA-A*03, and the E6 targeted T-cell subpopulation is primed and expanded with E6-derived peptides comprising the peptides of Table 283 (Seq. ID. Nos. 2423-2427) and at least one additional set of peptides based on the donor cell source HLA-A profile, wherein the at least one additional set of peptides are selected from the peptides of Tables 281-282 and 284-287. In some embodiments, the E6-derived peptides also include one or more sets of HLA-B and HLA-DR restricted peptides selected from Tables 288-300 (Seq. ID Nos. 2448-2512).

TABLE 283
HPV Strain 16 E6 HLA-A*03 Epitope Peptides

| SEQ ID NO. | Sequence |
|---|---|
| 2423 | LLIRCINCQK |
| 2424 | DIILECVYCK |
| 2425 | CVYCKQQLLR |
| 2426 | SLYGTTLEQQ |
| 2427 | IVYRDGNPY |

In some embodiments, the donor cell source is HLA-A*11:01, and the E6 targeted T-cell subpopulation is primed and expanded with one or more E6-derived peptides selected from Table 284 (Seq. ID. Nos. 2428-2432). In some embodiments, the donor cell source is HLA-A*11:01, and the E6 targeted T-cell subpopulation is primed and expanded with E6-derived peptides selected from Table 284 (Seq. ID. Nos. 2428-2432). In some embodiments, the donor cell source is HLA-A*11:01, and the E6 targeted T-cell subpopulation is primed and expanded with E6-derived peptides comprising the peptides of Table 284 (Seq. ID. Nos. 2428-2432). In some embodiments, the donor cell source is HLA-A*11:01, and the E6 targeted T-cell subpopulation is primed and expanded with E6-derived peptides comprising the peptides of Table 284 (Seq. ID. Nos. 2428-2432), and at least one additional set of peptides based on the donor cell source HLA-A profile, wherein the at least one additional set of peptides are selected from the peptides of Tables 281-283 and 285-287. In some embodiments, the E6-derived peptides also include one or more sets of HLA-B and HLA-DR restricted peptides selected from Tables 288-300 (Seq. ID Nos. 2448-2512).

TABLE 284
HPV Strain 16 E6 HLA-A*11:01 Epitope Peptides

| SEQ ID NO. | Sequence |
|---|---|
| 2428 | CVYCKQQLLR |
| 2429 | GTTLEQQYNK |
| 2430 | DIILECVYCK |
| 2431 | AFRDLCIVYR |
| 2432 | WTGRCMSCCR |

In some embodiments, the donor cell source is HLA-A*24:02, and the E6 targeted T-cell subpopulation is primed and expanded with one or more E6-derived peptides selected from Table 285 (Seq. ID. Nos. 2433-2437). In some embodiments, the donor cell source is HLA-A*24:02, and the E6 targeted T-cell subpopulation is primed and expanded with E6-derived peptides selected from Table 285 (Seq. ID. Nos. 2433-2437). In some embodiments, the donor cell source is HLA-A*24:02, and the E6 targeted T-cell subpopulation is primed and expanded with E6-derived peptides comprising the peptides of Table 285 (Seq. ID. Nos. 2433-2437). In some embodiments, the donor cell source is HLA-A*24:02, and the E6 targeted T-cell subpopulation is primed and expanded with E6-derived peptides comprising the peptides of Table 285 (Seq. ID. Nos. 2433-2437), and at least one additional set of peptides based on the donor cell source HLA-A profile, wherein the at least one additional set of peptides are selected from the peptides of Tables 281-284 and 286-287. In some embodiments, the E6-derived peptides also include one or more sets of HLA-B and HLA-DR restricted peptides selected from Tables 288-300 (Seq. ID Nos. 2448-2512).

TABLE 285
HPV Strain 16 E6 HLA-A*24:02 Epitope Peptides

| SEQ ID NO. | Sequence |
|---|---|
| 2433 | QYNKPLCDLL |
| 2434 | QDPQERPRKL |
| 2435 | LCPEEKQRHL |
| 2436 | VYDFAFRDL |
| 2437 | PYAVCDKCL |

In some embodiments, the donor cell source is HLA-A*26, and the E6 targeted T-cell subpopulation is primed and expanded with one or more E6-derived peptides selected from Table 286 (Seq. ID. Nos. 2438-2442). In some embodiments, the donor cell source is HLA-A*26, and the E6 targeted T-cell subpopulation is primed and expanded with E6-derived peptides selected from Table 286 (Seq. ID. Nos. 2438-2442). In some embodiments, the donor cell source is HLA-A*26, and the E6 targeted T-cell subpopulation is primed and expanded with E6-derived peptides comprising the peptides of Table 286 (Seq. ID. Nos. 2438-2442). In some embodiments, the donor cell source is HLA-A*26, and the E6 targeted T-cell subpopulation is primed and expanded with E6-derived peptides comprising the peptides of Table 286 (Seq. ID. Nos. 2438-2442) and at least one additional set of peptides based on the donor cell source HLA-A profile, wherein the at least one additional set of peptides are selected from the peptides of Tables 281-285 and 287. In some embodiments, the E6-derived peptides also include one or more sets of HLA-B and HLA-DR restricted peptides selected from Tables 288-300 (Seq. ID Nos. 2448-2512).

TABLE 286
HPV Strain 16 E6 HLA-A*26 Epitope Peptides

| SEQ ID NO. | Sequence |
|---|---|
| 2438 | EVYDFAFRDL |
| 2439 | AVCDKCLKFY |
| 2440 | EYRHYCYSLY |
| 2441 | DIILECVYCK |
| 2442 | CIVYRDGNPY |

In some embodiments, the donor cell source is HLA-A*68:01, and the E6 targeted T-cell subpopulation is primed and expanded with one or more E6-derived peptides selected from Table 287 (Seq. ID. Nos. 2443-2447). In some embodiments, the donor cell source is HLA-A*68:01, and the E6 targeted T-cell subpopulation is primed and expanded with E6-derived peptides selected from Table 287 (Seq. ID. Nos. 2443-2447). In some embodiments, the donor cell source is HLA-A*68:01, and the E6 targeted T-cell subpopulation is primed and expanded with E6-derived peptides comprising the peptides of Table 287 (Seq. ID. Nos. 2443-2447). In some embodiments, the donor cell source is HLA-A*68:01, and the E6 targeted T-cell subpopulation is primed and expanded with E6-derived peptides comprising the peptides of Table 287 (Seq. ID. Nos. 2443-2447), and at least one additional set of peptides based on the donor cell source HLA-A profile, wherein the at least one additional set of peptides are selected from the peptides of Tables 281-286. In some embodiments, the E6-derived peptides also include one or more sets of HLA-B and HLA-DR restricted peptides selected from Tables 288-300 (Seq. ID Nos. 2448-2512).

TABLE 287
HPV Strain 16 E6 HLA-A*68:01 Epitope
Peptides

| SEQ ID NO. | Sequence |
|---|---|
| 2443 | FAFRDLCIVYR |
| 2444 | RTAMFQDPQER |
| 2445 | CVYCKQQLLRR |
| 2446 | MSCCRSSRTRR |
| 2447 | DLLIRCINCQK |

In some embodiments, the donor cell source is HLA-B*07:02, and the E6 targeted T-cell subpopulation is primed and expanded with one or more E6-derived peptides selected from Table 288 (Seq. ID. Nos. 2448-2452). In some embodiments, the donor cell source is HLA-B*07:02, and the E6 targeted T-cell subpopulation is primed and expanded with E6-derived peptides selected from Table 288 (Seq. ID. Nos. 2448-2452). In some embodiments, the donor cell source is HLA-B*07:02, and the E6 targeted T-cell subpopulation is primed and expanded with E6-derived peptides comprising the peptides of Table 288 (Seq. ID. Nos. 2448-2452). In some embodiments, the donor cell source is HLA-B*07:02, and the E6 targeted T-cell subpopulation is primed and expanded with E6-derived peptides comprising the peptides of Table 288 (Seq. ID. Nos. 2448-2452), and at least one additional set of peptides based on the donor cell source HLA-B profile, wherein the at least one additional set of peptides are selected from the peptides of Tables 289-294. In some embodiments, the E6-derived peptides also include one or more sets of HLA-A and HLA-DR restricted peptides selected from Tables 281-287 and 295-300 (Seq. ID Nos. 2413-2447 and 2483-2512).

TABLE 288
HPV Strain 16 E6 HLA-B*07:02 Epitope Peptides

| SEQ ID NO. | Sequence |
|---|---|
| 2448 | RPRKLPQLCT |
| 2449 | NPYAVCDKCL |
| 2450 | LPQLCTELQT |
| 2451 | QERPRKLPQL |
| 2452 | DPQERPRKL |

In some embodiments, the donor cell source is HLA-B*08, and the E6 targeted T-cell subpopulation is primed and expanded with one or more E6-derived peptides selected from Table 289 (Seq. ID. Nos. 2453-2457). In some embodiments, the donor cell source is HLA-B*08, and the E6 targeted T-cell subpopulation is primed and expanded with E6-derived peptides selected from Table 289 (Seq. ID. Nos. 2453-2457). In some embodiments, the donor cell source is HLA-B*08, and the E6 targeted T-cell subpopulation is primed and expanded with E6-derived peptides comprising the peptides of Table 289 (Seq. ID. Nos. 2453-2457). In some embodiments, the donor cell source is HLA-B*08, and the E6 targeted T-cell subpopulation is primed and expanded with E6-derived peptides comprising the peptides of Table 289 (Seq. ID. Nos. 2453-2457) and at least one additional set of peptides based on the donor cell source HLA-B profile, wherein the at least one additional set of peptides are selected from the peptides of Tables 288 and 290-294. In some embodiments, the E6-derived peptides also include one or more sets of HLA-A and HLA-DR restricted peptides selected from Tables 281-287 and 295-300 (Seq. ID Nos. 2413-2447 and 2483-2512).

TABLE 289
HPV Strain 16 E6 HLA-B*08 Epitope Peptides

| SEQ ID NO. | Sequence |
|---|---|
| 2453 | CPEEKQRHL |
| 2454 | DPQERPRKL |
| 2455 | DKKQRFHNI |
| 2456 | ERPRKLPQL |
| 2457 | CVYCKQQLL |

In some embodiments, the donor cell source is HLA-B*15:01, and the E6 targeted T-cell subpopulation is primed and expanded with one or more E6-derived peptides selected from Table 290 (Seq. ID. Nos. 2458-2462). In some embodiments, the donor cell source is HLA-B*15:01, and the E6 targeted T-cell subpopulation is primed and expanded with E6-derived peptides selected from Table 290 (Seq. ID. Nos. 2458-2462). In some embodiments, the donor cell source is HLA-B*15:01, and the E6 targeted T-cell subpopulation is primed and expanded with E6-derived peptides comprising the peptides of Table 290 (Seq. ID. Nos. 2458-2462). In some embodiments, the donor cell source is HLA-B*15:01, and the E6 targeted T-cell subpopulation is primed and expanded with E6-derived peptides comprising the peptides of Table 290 (Seq. ID. Nos2458-2462) and at least one additional set of peptides based on the donor cell source HLA-B profile, wherein the at least one additional set of peptides are selected from the peptides of Tables 288-289 and 291-294. In some embodiments, the E6-derived peptides also include one or more sets of HLA-A and HLA-DR restricted peptides selected from Tables 281-287 and 295-300 (Seq. ID Nos. 2413-2447 and 2483-2512).

TABLE 290
HPV Strain 16 E6 HLA-B*15:01 (B62)
Epitope Peptides

| SEQ ID NO. | Sequence |
|---|---|
| 2458 | KQQLLRREVY |
| 2459 | AVCDKCLKFY |
| 2460 | QLLRREVYDF |
| 2461 | QLCTELQTTI |
| 2462 | FAFRDLCIVY |

In some embodiments, the donor cell source is HLA-B*18, and the E6 targeted T-cell subpopulation is primed and expanded with one or more E6-derived peptides selected from Table 291 (Seq. ID. Nos. 2463-2467). In some embodiments, the donor cell source is HLA-B*18, and the E6 targeted T-cell subpopulation is primed and expanded with E6-derived peptides selected from Table 291 (Seq. ID. Nos. 2463-2467). In some embodiments, the donor cell source is HLA-B*18, and the E6 targeted T-cell subpopulation is primed and expanded with E6-derived peptides comprising the peptides of Table 291 (Seq. ID. Nos. 2463-2467). In some embodiments, the donor cell source is HLA-B*18, and the E6 targeted T-cell subpopulation is primed and expanded with E6-derived peptides comprising the peptides of Table 291 (Seq. ID. Nos. 2463-2467) and at least one additional set of peptides based on the donor cell source HLA-B profile, wherein the at least one additional set of peptides are selected from the peptides of Tables 288-290 and 292-294. In some embodiments, the E6-derived peptides also include one or more sets of HLA-A and HLA-DR restricted peptides selected from Tables 281-287 and 295-300 (Seq. ID Nos. 2413-2447 and 2483-2512).

TABLE 291
HPV Strain 16 E6 HLA-B*18 Epitope Peptides

| SEQ ID NO. | Sequence |
|---|---|
| 2463 | LEQQYNKPL |
| 2464 | QQLLRREVY |
| 2465 | DPQERPRKL |
| 2466 | AFRDLCIVY |
| 2467 | REVYDFAF |

In some embodiments, the donor cell source is HLA-B*27:05, and the E6 targeted T-cell subpopulation is primed and expanded with one or more E6-derived peptides selected from Table 292 (Seq. ID. Nos. 2468-2472). In some embodiments, the donor cell source is HLA-B*27:05, and the E6 targeted T-cell subpopulation is primed and expanded with E6-derived peptides selected from Table 292 (Seq. ID. Nos. 2468-2472). In some embodiments, the donor cell source is HLA-B*27:05, and the E6 targeted T-cell subpopulation is primed and expanded with E6-derived peptides comprising the peptides of Table 292 (Seq. ID. Nos. 2468-2472). In some embodiments, the donor cell source is HLA-B*27:05, and the E6 targeted T-cell subpopulation is primed and expanded with E6-derived peptides comprising the peptides of Table 292 (Seq. ID. Nos. 2468-2472) and at least one additional set of peptides based on the donor cell source HLA-B profile, wherein the at least one additional set of peptides are selected from the peptides of Tables 288-291 and 293-294. In some embodiments, the E6-derived peptides also include one or more sets of HLA-A and HLA-DR restricted peptides selected from 281-287 and 295-300 (Seq. ID Nos. 2413-2447 and 2483-2512).

TABLE 292
HPV Strain 16 E6 HLA-B*27:05 Epitope Peptides

| SEQ ID NO. | Sequence |
|---|---|
| 2468 | RREVYDFAFR |
| 2469 | QRHLDKKQRF |
| 2470 | IRCINCQKPL |
| 2471 | LRREVYDFAF |
| 2472 | RKLPQLCTEL |

In some embodiments, the donor cell source is HLA-B*35:01, and the E6 targeted T-cell subpopulation is primed and expanded with one or more E6-derived peptides selected from Table 293 (Seq. ID. Nos. 2473-2477). In some embodiments, the donor cell source is HLA-B*35:01, and the E6 targeted T-cell subpopulation is primed and expanded with E6-derived peptides selected from Table 293 (Seq. ID. Nos. 2473-2477). In some embodiments, the donor cell source is HLA-B*35:01, and the E6 targeted T-cell subpopulation is primed and expanded with E6-derived peptides comprising the peptides of Table 293 (Seq. ID. Nos. 2473-2477). In some embodiments, the donor cell source is HLA-B*35:01, and the E6 targeted T-cell subpopulation is primed and expanded with E6-derived peptides comprising the peptides of Table 293 (Seq. ID. Nos. 2473-2477) and at least one additional set of peptides based on the donor cell source HLA-B profile, wherein the at least one additional set of peptides are selected from the peptides of Tables 288-292 and 294. In some embodiments, the E6-derived peptides also include one or more sets of HLA-A and HLA-DR restricted peptides selected from 281-287 and 295-300 (Seq. ID Nos. 2413-2447 and 2483-2512).

TABLE 293
HPV Strain 16 E6 HLA-B*35:01 Epitope Peptides

| SEQ ID NO. | Sequence |
|---|---|
| 2473 | NPYAVCDKCL |
| 2474 | FAFRDLCIVY |
| 2475 | LQTTIHDIIL |
| 2476 | KPLCDLLIRC |
| 2477 | ECVYCKQQLL |

In some embodiments, the donor cell source is HLA-B*58:02, and the E6 targeted T-cell subpopulation is primed and expanded with one or more E6-derived peptides selected from Table 294 (Seq. ID. Nos. 2478-2482). In some embodiments, the donor cell source is HLA-B*58:02, and the E6 targeted T-cell subpopulation is primed and expanded with E6-derived peptides selected from Table 294 (Seq. ID. Nos. 2478-2482). In some embodiments, the donor cell source is HLA-B*58:02, and the E6 targeted T-cell subpopulation is primed and expanded with E6-derived peptides comprising the peptides of Table 294 (Seq. ID. Nos. 2478-2482). In some embodiments, the donor cell source is HLA-B*58:02, and the E6 targeted T-cell subpopulation is primed and expanded with E6-derived peptides comprising the peptides of Table 294 (Seq. ID. Nos. 2478-2482) and at least one additional set of peptides based on the donor cell source HLA-B profile, wherein the at least one additional set of peptides are selected from the peptides of Tables 288-293. In some embodiments, the E6-derived peptides also include one or more sets of HLA-A and HLA-DR restricted peptides selected from Tables 281-287 and 295-300 (Seq. ID Nos. 2413-2447 and 2483-2512).

TABLE 294
HPV Strain 16 E6 HLA-B*58:02 Epitope Peptides

| SEQ ID NO. | Sequence |
|---|---|
| 2478 | YSKISEYRHY |
| 2479 | EVYDFAFRDL |
| 2480 | SSRTRRETQL |
| 2481 | FAFRDLCIVY |
| 2482 | YAVCDKCLKF |

In some embodiments, the donor cell source is HLA-DRB1*0101, and the E6 targeted T-cell subpopulation is primed and expanded with one or more E6-derived peptides selected from Table 295 (Seq. ID. Nos. 2483-2487). In some embodiments, the donor cell source is HLA-DRB1*0101, and the E6 targeted T-cell subpopulation is primed and expanded with E6-derived peptides selected from Table 295 (Seq. ID. Nos. 2483-2487). In some embodiments, the donor cell source is HLA-DRB1*0101, and the E6 targeted T-cell subpopulation is primed and expanded with E6-derived peptides comprising the peptides of Table 295 (Seq. ID. Nos. 2483-2487). In some embodiments, the donor cell source is HLA-DRB1*0101, and the E6 targeted T-cell subpopulation is primed and expanded with E6-derived peptides comprising the peptides of Table 295 (Seq. ID. Nos. 2483-2487) and at least one additional set of peptides based on the donor cell source HLA-DR profile, wherein the at least one additional set of peptides are selected from the peptides of Tables 296-300. In some embodiments, the E6-derived peptides also include one or more sets of HLA-A and HLA-B restricted peptides selected from Tables 281-294 (Seq. ID Nos. 2413-2482).

TABLE 295
HPV Strain 16 E6 HLA-DRB1*0101 Epitope Peptides

| SEQ ID NO. | Sequence |
|---|---|
| 2483 | IRCINCQKPLCPEEK |
| 2484 | CIVYRDGNPYAVCDK |
| 2485 | RHYCYSLYGTTLEQQ |
| 2486 | NKPLCDLLIRCINCQ |
| 2487 | DLLIRCINCQKPLCP |

In some embodiments, the donor cell source is HLA-DRB1*0301, and the E6 targeted T-cell subpopulation is primed and expanded with one or more E6-derived peptides selected from Table 296 (Seq. ID. Nos. 2488-2492). In some embodiments, the donor cell source is HLA-DRB1*0301, and the E6 targeted T-cell subpopulation is primed and expanded with E6-derived peptides selected from Table 296 (Seq. ID. Nos. 2488-2492). In some embodiments, the donor cell source is HLA-DRB1*0301, and the E6 targeted T-cell subpopulation is primed and expanded with E6-derived peptides comprising the peptides of Table 296 (Seq. ID. Nos. 2488-2492). In some embodiments, the donor cell source is HLA-DRB1*0301, and the E6 targeted T-cell subpopulation is primed and expanded with E6-derived peptides comprising the peptides of Table 296 (Seq. ID. Nos. 2488-2492) and at least one additional set of peptides based on the donor cell source HLA-DR profile, wherein the at least one additional set of peptides are selected from the peptides of Tables 295 and 297-300. In some embodiments, the E6-derived peptides also include one or more sets of HLA-A and HLA-B restricted peptides selected from Tables 281-294 (Seq. ID Nos. 2211-2280).

TABLE 296
HPV Strain 16 E6 HLA-DRB1*0301 (DR17)
Epitope Peptides

| SEQ ID NO. | Sequence |
|---|---|
| 2488 | RTAMFQDPQERPRKL |
| 2489 | LCIVYRDGNPYAVCD |
| 2490 | EKQRHLDKKQRFHNI |
| 2491 | GTTLEQQYNKPLCDL |
| 2492 | NPYAVCDKCLKFYSK |

In some embodiments, the donor cell source is HLA-DRB1*0401, and the E6 targeted T-cell subpopulation is primed and expanded with one or more E6-derived peptides selected from Table 297 (Seq. ID. Nos. 2493-2497). In some embodiments, the donor cell source is HLA-DRB1*0401, and the E6 targeted T-cell subpopulation is primed and expanded with E6-derived peptides selected from Table 297 (Seq. ID. Nos. 2493-2497). In some embodiments, the donor cell source is HLA-DRB1*0401, and the E6 targeted T-cell subpopulation is primed and expanded with E6-derived peptides comprising the peptides of Table 297 (Seq. ID. Nos. 2493-2497). In some embodiments, the donor cell source is HLA-DRB1*0401, and the E6 targeted T-cell subpopulation is primed and expanded with E6-derived peptides comprising the peptides of Table 297 (Seq. ID. Nos. 2493-2497) and at least one additional set of peptides based on the donor cell source HLA-DR profile, wherein the at least one additional set of peptides are selected from the peptides of Tables 295-296 and 298-300. In some embodiments, the E6-derived peptides also include one or more sets of HLA-A and HLA-B restricted peptides selected from Tables 281-294 (Seq. ID Nos. 2413-2482).

TABLE 297
HPV Strain 16 E6 HLA-DRB1*0401 (DR4Dw4)
Epitope Peptides

| SEQ ID NO. | Sequence |
|---|---|
| 2493 | PRKLPQLCTELQTTI |
| 2494 | LPQLCTELQTTIHDI |
| 2495 | FRDLCIVYRDGNPYA |
| 2496 | LCIVYRDGNPYAVCD |
| 2497 | REVYDFAFRDLCIVY |

In some embodiments, the donor cell source is HLA-DRB1*0701, and the E6 targeted T-cell subpopulation is primed and expanded with one or more E6-derived peptides selected from Table 298 (Seq. ID. Nos. 2498-2502). In some embodiments, the donor cell source is HLA-DRB1*0701, and the E6 targeted T-cell subpopulation is primed and expanded with E6-derived peptides selected from Table 298 (Seq. ID. Nos. 2498-2502). In some embodiments, the donor cell source is HLA-DRB1*0701, and the E6 targeted T-cell subpopulation is primed and expanded with E6-derived peptides comprising the peptides of Table 298 (Seq. ID. Nos. 2498-2502). In some embodiments, the donor cell source is HLA-DRB1*0701, and the E6 targeted T-cell subpopulation is primed and expanded with E6-derived peptides comprising the peptides of Table 298 (Seq. ID. Nos. 2498-2502) and at least one additional set of peptides based on the donor cell source HLA-DR profile, wherein the at least one additional set of peptides are selected from the peptides of Tables 295-297 and 299-300. In some embodiments, the E6-derived peptides also include one or more sets of HLA-A and HLA-B restricted peptides selected from Tables 281-294 (Seq. ID Nos. 2413-2482).

TABLE 298
HPV Strain 16 E6 HLA-DRB1*0701 Epitope Peptides

| SEQ ID NO. | Sequence |
|---|---|
| 2498 | YSLYGTTLEQQYNKP |
| 2499 | CTELQTTIHDIILEC |
| 2500 | QTTIHDIILECVYCK |
| 2501 | LKFYSKISEYRHYCY |
| 2502 | GTTLEQQYNKPLCDL |

In some embodiments, the donor cell source is HLA-DRB1*1101, and the E6 targeted T-cell subpopulation is primed and expanded with one or more E6-derived peptides selected from Table 299 (Seq. ID. Nos. 2503-2507). In some embodiments, the donor cell source is HLA-DRB1*1101, and the E6 targeted T-cell subpopulation is primed and expanded with E6-derived peptides selected from Table 299 (Seq. ID. Nos. 2503-2507). In some embodiments, the donor cell source is HLA-DRB1*1101, and the E6 targeted T-cell subpopulation is primed and expanded with E6-derived peptides comprising the peptides of Table 299 (Seq. ID. 2503-2507). In some embodiments, the donor cell source is HLA-DRB1*1101, and the E6 targeted T-cell subpopulation is primed and expanded with E6-derived peptides comprising the peptides of Table 299 (Seq. ID. Nos. 2503-2507) and at least one additional set of peptides based on the donor cell source HLA-DR profile, wherein the at least one additional set of peptides are selected from the peptides of Tables 295-298 and 300. In some embodiments, the E6-derived peptides also include one or more sets of HLA-A and HLA-B restricted peptides selected from Tables 281-294 (Seq. ID Nos. 2413-2482).

TABLE 299
HPV Strain 16 E6 HLA-DRB1*1101 Epitope Peptides

| SEQ ID NO. | Sequence |
|---|---|
| 2503 | FRDLCIVYRDGNPYA |
| 2504 | GNPYAVCDKCLKFYS |
| 2505 | REVYDFAFRDLCIVY |
| 2506 | DLLIRCINCQKPLCP |
| 2507 | LKFYSKISEYRHYCY |

In some embodiments, the donor cell source is HLA-DRB1*1501, and the E6 targeted T-cell subpopulation is primed and expanded with one or more E6-derived peptides selected from Table 300 (Seq. ID. Nos. 2508-2512). In some embodiments, the donor cell source is HLA-DRB1*1501, and the E6 targeted T-cell subpopulation is primed and expanded with E6-derived peptides selected from Table 300 (Seq. ID. Nos. 2508-2512). In some embodiments, the donor cell source is HLA-DRB1*1501, and the E6 targeted T-cell subpopulation is primed and expanded with E6-derived peptides comprising the peptides of Table 300 (Seq. ID. Nos. 2508-2512). In some embodiments, the donor cell source is HLA-DRB1*1501, and the E6 targeted T-cell subpopulation is primed and expanded with E6-derived peptides comprising the peptides of Table 300 (Seq. ID. Nos. 2508-2512) and at least one additional set of peptides based on the donor cell source HLA-DR profile, wherein the at least one additional set of peptides are selected from the peptides of Tables 295-299. In some embodiments, the E6-derived peptides also include one or more sets of HLA-A and HLA-B restricted peptides selected from Tables 281-294 (Seq. ID Nos. 2413-2482).

TABLE 300
HPV Strain 16 E6 HLA-DRB1*1501 (DR2b)
Epitope Peptides

| SEQ ID NO. | Sequence |
|---|---|
| 2508 | DKCLKFYSKISEYRH |
| 2509 | RREVYDFAFRDLCIV |
| 2510 | IRCINCQKPLCPEEK |
| 2511 | LDKKQRFHNIRGRWT |
| 2512 | LECVYCKQQLLRREV |

Human Papillomavirus (HPV) Strain 16 E6 Antigenic Peptides

In some embodiments, the MUSTANG composition includes Human Papillomavirus (HPV) Strain 16 E7 specific T-cells. E7 specific T-cells can be generated as described below using one or more antigenic peptides to E7. In some embodiments, the E7 specific T-cells are generated using one or more antigenic peptides to E7, or a modified or heteroclitic peptide derived from a E7 peptide. In some embodiments, E7 specific T-cells are generated using a E7 antigen library comprising a pool of peptides (for example 15mers) containing amino acid overlap (for example 11 amino acids of overlap) between each sequence formed by scanning the protein amino acid sequence SEQ. ID. No. 2513 (UniProt KB-P03129) for HPV Strain 16-8 E7:

MHGDTPTLHEYMLDLQPETTDLYCYEQLNDSSEEEDEIDGPAGQAEPDRA
HYNIVTFCCKCDSTLRLCVQSTHVDIRTLEDLLMGTLGIVCPICSQKP.

In some embodiments, the E7 specific T-cells are generated using one or more antigenic peptides to E7, or a modified or heteroclitic peptide derived from a E7 peptide. In some embodiments, the E7 specific T-cells are generated with peptides that recognize class I MHC molecules. In some embodiments, the E7 specific T-cells are generated with peptides that recognize class II MEW molecules. In some embodiments, the E7 specific T-cells are generated with peptides that recognize both class I and class II MHC molecules.

In some embodiments, the E7 peptides used to prime and expand a T-cell subpopulation includes specifically selected HLA-restricted peptides generated by determining the HLA profile of the donor source, and including peptides derived from E7 that best match the donor's HLA. In some embodiments, the E7 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting E7 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 301-307, the HLA-B peptides are selected from the peptides of Tables 308-314, and the HLA-DR peptides are selected from the peptides of Tables 315-320. For example, if the donor cell source has an HLA profile that is HLA-A*01/*02:01; HLA-B*15:01/*18; and HLA-DRB1*0101/*0301, then the E7 peptides used to prime and expand the E7 specific T-cell subpopulation are restricted to the specific HLA profile, and may include the peptides identified in Table 301 (Seq. ID. Nos. 2514-2518) for HLA-A*01; Table 302 (Seq. ID. Nos. 2519-2523) for HLA-A*02:01; Table 310 (Seq. ID. Nos. 2559-2563) for HLA-B*15:01; Table 311 (Seq. ID. Nos. 2564-2568) for HLA-B*18; Table 315 (Seq. ID. Nos. 2584-2588) for HLA-DRB1*0101; and Table 316 (Seq. ID. Nos. 2589-2593) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the E7 targeted T-cell subpopulation is primed and expanded with one or more E7-derived peptides selected from Table 301 (Seq. ID. Nos. 2514-2518). In some embodiments, the donor cell source is HLA-A*01, and the E7 targeted T-cell subpopulation is primed and expanded with E7-derived peptides selected from Table 301 (Seq. ID. Nos. 2514-2518). In some embodiments, the donor cell source is HLA-A*01, and the E7 targeted T-cell subpopulation is primed and expanded with E7-derived peptides comprising the peptides of from Table 301 (Seq. ID. Nos. 2514-2518). In some embodiments, the donor cell source is HLA-A*01, and the E7 targeted T-cell subpopulation is primed and expanded with E7-derived peptides comprising the peptides of from Table 301 (Seq. ID. Nos. 2514-2518) and at least one additional set of peptides based on the donor cell source HLA-A profile, wherein the at least one additional set of peptides are selected from the peptides of Tables 302-307. In some embodiments, the E7-derived peptides also include one or more sets of HLA-B and HLA-DR restricted peptides selected from Tables 308-320 (Seq. ID Nos. 2547-2613).

TABLE 301
HPV Strain 16 E7 HLA-A*01 Epitope Peptides

| SEQ ID NO. | Sequence |
|---|---|
| 2514 | MHGDTPTLHEY |
| 2515 | HGDTPTLHEY |
| 2516 | QPETTDLYCY |
| 2517 | DLQPETTDLY |
| 2518 | QAEPDRAHY |

In some embodiments, the donor cell source is HLA-A*02:01, and the E7 targeted T-cell subpopulation is primed and expanded with one or more E7-derived peptides selected from Table 302 (Seq. ID. Nos. 2519-2523). In some embodiments, the donor cell source is HLA-A*02:01, and the E7 targeted T-cell subpopulation is primed and expanded with E7-derived peptides selected from Table 302 (Seq. ID. Nos. 2519-2523). In some embodiments, the donor cell source is HLA-A*02:01, and the E7 targeted T-cell subpopulation is primed and expanded with E7-derived peptides comprising the peptides of Table 302 (Seq. ID. Nos. 2519-2523). In some embodiments, the donor cell source is HLA-A*02:01, and the E7 targeted T-cell subpopulation is primed and expanded with E7-derived peptides comprising the peptides of Table 302 (Seq. ID. Nos. 2519-2523) and at least one additional set of peptides based on the donor cell source HLA-A profile, wherein the at least one additional set of peptides are selected from the peptides of Tables 301, and 303-307. In some embodiments, the E7-derived peptides also include one or more sets of HLA-B and HLA-DR restricted peptides selected from Tables 308-320 (Seq. ID Nos. 2547-2613).

TABLE 302
HPV Strain 16 E7 HLA-A*02:01 Epitope Peptides

| SEQ ID NO. | Sequence |
|---|---|
| 2519 | DLLMGTLGIV |
| 2520 | TLEDLLMGTL |
| 2521 | LLMGTLGIV |
| 2522 | TLHEYMLDL |
| 2523 | DLQPETTDL |

In some embodiments, the donor cell source is HLA-A*03, and the E7 targeted T-cell subpopulation is primed and expanded with one or more E7-derived peptides selected from Table 303 (Seq. ID. Nos. 2524-2427). In some embodiments, the donor cell source is HLA-A*03, and the E7 targeted T-cell subpopulation is primed and expanded with E7-derived peptides selected from Table 303 (Seq. ID. Nos. 2524-2427). In some embodiments, the donor cell source is HLA-A*03, and the E7 targeted T-cell subpopulation is primed and expanded with E7-derived peptides comprising the peptides of Table 303 (Seq. ID. Nos. 2524-2427). In some embodiments, the donor cell source is HLA-A*03, and the E7 targeted T-cell subpopulation is primed and expanded with E7-derived peptides comprising the peptides of Table 303 (Seq. ID. Nos. 2524-2427) and at least one additional set of peptides based on the donor cell source HLA-A profile, wherein the at least one additional set of peptides are selected from the peptides of Tables 301-302 and 304-307. In some embodiments, the E7-derived peptides also include one or more sets of HLA-B and HLA-DR restricted peptides selected from Tables 308-320 (Seq. ID Nos. 2547-2613).

TABLE 303
HPV Strain 16 E7 HLA-A*03 Epitope Peptides

| SEQ ID NO. | Sequence |
|---|---|
| 2524 | TLRLCVQSTH |
| 2525 | GIVCPICSQK |
| 2526 | DLQPETTDLY |
| 2527 | LLMGTLGIVC |
| 2528 | IVCPICSQK |

In some embodiments, the donor cell source is HLA-A*11:01, and the E7 targeted T-cell subpopulation is primed and expanded with one or more E7-derived peptides selected from Table 304 (Seq. ID. Nos. 2529-2533). In some embodiments, the donor cell source is HLA-A*11:01, and the E7 targeted T-cell subpopulation is primed and expanded with E7-derived peptides selected from Table 304 (Seq. ID. Nos. 2529-2533). In some embodiments, the donor cell source is HLA-A*11:01, and the E7 targeted T-cell subpopulation is primed and expanded with E7-derived peptides comprising the peptides of Table 304 (Seq. ID. Nos. 2529-2533). In some embodiments, the donor cell source is HLA-A*11:01, and the E7 targeted T-cell subpopulation is primed and expanded with E7-derived peptides comprising the peptides of Table 304 (Seq. ID. Nos. 2529-2533), and at least one additional set of peptides based on the donor cell source HLA-A profile, wherein the at least one additional set of peptides are selected from the peptides of Tables 301-303 and 305-307. In some embodiments, the E7-derived peptides also include one or more sets of HLA-B and HLA-DR restricted peptides selected from Tables 308-320 (Seq. ID Nos. 2547-2613).

TABLE 304
HPV Strain 16 E7 HLA-A*11:01 Epitope Peptides

| SEQ ID NO. | Sequence |
|---|---|
| 2529 | CVQSTHVDIR |
| 2530 | GIVCPICSQK |
| 2531 | SSEEEDEIDG |
| 2532 | IVCPICSQK |
| 2533 | STLRLCVQS |

In some embodiments, the donor cell source is HLA-A*24:02, and the E7 targeted T-cell subpopulation is primed and expanded with one or more E7-derived peptides selected from Table 305 (Seq. ID. Nos. 2534-2538). In some embodiments, the donor cell source is HLA-A*24:02, and the E7 targeted T-cell subpopulation is primed and expanded with E7-derived peptides selected from Table 305 (Seq. ID. Nos. 2534-2538). In some embodiments, the donor cell source is HLA-A*24:02, and the E7 targeted T-cell subpopulation is primed and expanded with E7-derived peptides comprising the peptides of Table 305 (Seq. ID. Nos. 2534-2538). In some embodiments, the donor cell source is HLA-A*24:02, and the E7 targeted T-cell subpopulation is primed and expanded with E7-derived peptides comprising the peptides of Table 305 (Seq. ID. Nos. 2534-2538), and at least one additional set of peptides based on the donor cell source HLA-A profile, wherein the at least one additional set of peptides are selected from the peptides of Tables 301-304 and 306-307. In some embodiments, the E7-derived peptides also include one or more sets of HLA-B and HLA-DR restricted peptides selected from Tables 308-320 (Seq. ID Nos. 2547-2613).

TABLE 305
HPV Strain 16 E7 HLA-A*24:02 Epitope Peptides

| SEQ ID NO. | Sequence |
|---|---|
| 2534 | TFCCKCDSTL |
| 2535 | VDIRTLEDLL |
| 2536 | AEPDRAHYNI |
| 2537 | TDLYCYEQL |
| 2538 | GTLGIVCPI |

In some embodiments, the donor cell source is HLA-A*26, and the E7 targeted T-cell subpopulation is primed and expanded with one or more E7-derived peptides selected from Table 306 (Seq. ID. Nos. 2539-2543). In some embodiments, the donor cell source is HLA-A*26, and the E7 targeted T-cell subpopulation is primed and expanded with E7-derived peptides selected from Table 306 (Seq. ID. Nos. 2539-2543). In some embodiments, the donor cell source is HLA-A*26, and the E7 targeted T-cell subpopulation is primed and expanded with E7-derived peptides comprising the peptides of Table 306 (Seq. ID. Nos. 2539-2543). In some embodiments, the donor cell source is HLA-A*26, and the E7 targeted T-cell subpopulation is primed and expanded with E7-derived peptides comprising the peptides of Table 306 (Seq. ID. Nos. 2539-2543), and at least one additional set of peptides based on the donor cell source HLA-A profile, wherein the at least one additional set of peptides are selected from the peptides of Tables 301-305 and 307. In some embodiments, the E7-derived peptides also include one or more sets of HLA-B and HLA-DR restricted peptides selected from Tables 308-320 (Seq. ID Nos. 2547-2613).

TABLE 306
HPV Strain 16 E7 HLA-A*26 Epitope Peptides

| SEQ ID NO. | Sequence |
|---|---|
| 2539 | DTPTLHEYML |
| 2540 | HVDIRTLEDL |
| 2541 | DRAHYNIVTF |
| 2542 | STHVDIRTL |
| 2543 | ETTDLYCYE |

In some embodiments, the donor cell source is HLA-A*68:01, and the E7 targeted T-cell subpopulation is primed and expanded with one or more E7-derived peptides selected from Table 307 (Seq. ID. Nos. 2544-2548). In some embodiments, the donor cell source is HLA-A*68:01, and the E7 targeted T-cell subpopulation is primed and expanded with E7-derived peptides selected from Table 307 (Seq. ID. Nos. 2544-2548). In some embodiments, the donor cell source is HLA-A*68:01, and the E7 targeted T-cell subpopulation is primed and expanded with E7-derived peptides comprising the peptides of Table 307 (Seq. ID. Nos. 2544-2548). In some embodiments, the donor cell source is HLA-A*68:01, and the E7 targeted T-cell subpopulation is primed and expanded with E7-derived peptides comprising the peptides of Table 307 (Seq. ID. Nos. 2544-2548), and at least one additional set of peptides based on the donor cell source HLA-A profile, wherein the at least one additional set of peptides are selected from the peptides of Tables 301-306. In some embodiments, the E7-derived peptides also include one or more sets of HLA-B and HLA-DR restricted peptides selected from Tables 308-320 (Seq. ID Nos. 2447-2512).

TABLE 307
HPV Strain 16 E7 HLA-A*68:01 Epitope
Peptides

| SEQ ID NO. | Sequence |
|---|---|
| 2544 | TFCCKCDSTLR |
| 2545 | ETTDLYCYEQL |
| 2546 | CVQSTHVDIR |
| 2547 | IVCPICSQK |
| 2548 | PAGQAEPDR |

In some embodiments, the donor cell source is HLA-B*07:02, and the E7 targeted T-cell subpopulation is primed and expanded with one or more E7-derived peptides selected from Table 308 (Seq. ID. Nos. 2549-2553). In some embodiments, the donor cell source is HLA-B*07:02, and the E7 targeted T-cell subpopulation is primed and expanded with E7-derived peptides selected from Table 308 (Seq. ID. Nos. 2549-2553). In some embodiments, the donor cell source is HLA-B*07:02, and the E7 targeted T-cell subpopulation is primed and expanded with E7-derived peptides comprising the peptides of Table 308 (Seq. ID. Nos. 2549-2553). In some embodiments, the donor cell source is HLA-B*07:02, and the E7 targeted T-cell subpopulation is primed and expanded with E7-derived peptides comprising the peptides of Table 308 (Seq. ID. Nos. 2549-2553), and at least one additional set of peptides based on the donor cell source HLA-B profile, wherein the at least one additional set of peptides are selected from the peptides of Tables 309-314. In some embodiments, the E7-derived peptides also include one or more sets of HLA-A and HLA-DR restricted peptides selected from Tables 301-307 and 315-320 (Seq. ID Nos. 2514-2548 and 2584-2513).

TABLE 308
HPV Strain 16 E7 HLA-B*07:02 Epitope Peptides

| SEQ ID NO. | Sequence |
|---|---|
| 2549 | EPDRAHYNIV |
| 2550 | GPAGQAEPDR |
| 2551 | CCKCDSTLRL |
| 2552 | TPTLHEYML |
| 2553 | EIDGPAGQA |

In some embodiments, the donor cell source is HLA-B*08, and the E7 targeted T-cell subpopulation is primed and expanded with one or more E7-derived peptides selected from Table 309 (Seq. ID. Nos. 2554-2558). In some embodiments, the donor cell source is HLA-B*08, and the E7 targeted T-cell subpopulation is primed and expanded with E7-derived peptides selected from Table 309 (Seq. ID. Nos. 2554-2558). In some embodiments, the donor cell source is HLA-B*08, and the E7 targeted T-cell subpopulation is primed and expanded with E7-derived peptides comprising the peptides of Table 309 (Seq. ID. Nos. 2554-2558). In some embodiments, the donor cell source is HLA-B*08, and the E7 targeted T-cell subpopulation is primed and expanded with E7-derived peptides comprising the peptides of Table 309 (Seq. ID. Nos. 2554-2558) and at least one additional set of peptides based on the donor cell source HLA-B profile, wherein the at least one additional set of peptides are selected from the peptides of Tables 308 and 310-314. In some embodiments, the E7-derived peptides also include one or more sets of HLA-A and HLA-DR restricted peptides selected from Tables 301-307 and 315-320 (Seq. ID Nos. 2514-2548 and 2584-2513).

TABLE 309
HPV Strain 16 E7 HLA-B*08 Epitope Peptides

| SEQ ID NO. | Sequence |
|---|---|
| 2554 | DIRTLEDLL |
| 2555 | TLHEYMLDL |
| 2556 | TPTLHEYML |
| 2557 | DLQPETTDL |
| 2558 | CCKCDSTL |

In some embodiments, the donor cell source is HLA-B*15:01, and the E7 targeted T-cell subpopulation is primed and expanded with one or more E7-derived peptides selected from Table 310 (Seq. ID. Nos. 2559-2563). In some embodiments, the donor cell source is HLA-B*15:01, and the E7 targeted T-cell subpopulation is primed and expanded with E7-derived peptides selected from Table 310 (Seq. ID. Nos. 2559-2563). In some embodiments, the donor cell source is HLA-B*15:01, and the E7 targeted T-cell subpopulation is primed and expanded with E7-derived peptides comprising the peptides of Table 310 (Seq. ID. Nos. 2559-2563). In some embodiments, the donor cell source is HLA-B*15:01, and the E7 targeted T-cell subpopulation is primed and expanded with E7-derived peptides comprising the peptides of Table 310 (Seq. ID. Nos. 2559-2563) and at least one additional set of peptides based on the donor cell source HLA-B profile, wherein the at least one additional set of peptides are selected from the peptides of Tables 308-309 and 311-314. In some embodiments, the E7-derived peptides also include one or more sets of HLA-A and HLA-DR restricted peptides selected from Tables 301-307 and 315-320 (Seq. ID Nos. 2514-2548 and 2584-2513).

TABLE 310
HPV Strain 16 E7 HLA-B*15:01 (B62) Epitope Peptides

| SEQ ID NO. | Sequence |
|---|---|
| 2559 | DLQPETTDLY |
| 2560 | GQAEPDRAHY |
| 2561 | TLRLCVQSTH |
| 2562 | LLMGTLGIVC |
| 2563 | LQPETTDLY |

In some embodiments, the donor cell source is HLA-B*18, and the E7 targeted T-cell subpopulation is primed and expanded with one or more E7-derived peptides selected from Table 311 (Seq. ID. Nos. 2564-2568). In some embodiments, the donor cell source is HLA-B*18, and the E7 targeted T-cell subpopulation is primed and expanded with E7-derived peptides selected from Table 311 (Seq. ID. Nos. 2564-2568). In some embodiments, the donor cell source is HLA-B*18, and the E7 targeted T-cell subpopulation is primed and expanded with E7-derived peptides comprising the peptides of Table 311 (Seq. ID. Nos. 2564-2568). In some embodiments, the donor cell source is HLA-B*18, and the E7 targeted T-cell subpopulation is primed and expanded with E7-derived peptides comprising the peptides of Table 311 (Seq. ID. Nos. 2564-2568) and at least one additional set of peptides based on the donor cell source HLA-B profile, wherein the at least one additional set of peptides are selected from the peptides of Tables 308-310 and 312-314. In some embodiments, the E7-derived peptides also include one or more sets of HLA-A and HLA-DR restricted peptides selected from Tables 301-307 and 315-320 (Seq. ID Nos. 2514-2548 and 2584-2513).

TABLE 311
HPV Strain 16 E7 HLA-B*18 Epitope Peptides

| SEQ ID NO. | Sequence |
|---|---|
| 2564 | LEDLLMGTL |
| 2565 | PETTDLYCY |
| 2566 | DEIDGPAGQ |
| 2567 | DIRTLEDLL |
| 2568 | AEPDRAHY |

In some embodiments, the donor cell source is HLA-B*27:05, and the E7 targeted T-cell subpopulation is primed and expanded with one or more E7-derived peptides selected from Table 312 (Seq. ID. Nos. 2569-2573). In some embodiments, the donor cell source is HLA-B*27:05, and the E7 targeted T-cell subpopulation is primed and expanded with E7-derived peptides selected from Table 312 (Seq. ID. Nos. 2569-2573). In some embodiments, the donor cell source is HLA-B*27:05, and the E7 targeted T-cell subpopulation is primed and expanded with E7-derived peptides comprising the peptides of Table 312 (Seq. ID. Nos. 2569-2573). In some embodiments, the donor cell source is HLA-B*27:05, and the E7 targeted T-cell subpopulation is primed and expanded with E7-derived peptides comprising the peptides of Table 312 (Seq. ID. Nos. 2569-2573) and at least one additional set of peptides based on the donor cell source HLA-B profile, wherein the at least one additional set of peptides are selected from the peptides of Tables 308-314 and 313-314. In some embodiments, the E7-derived peptides also include one or more sets of HLA-A and HLA-DR restricted peptides selected from Tables 301-307 and 315-320 (Seq.

ID Nos. 2514-2548 and 2584-2513).

TABLE 312
HPV Strain 16 E7 HLA-B*27:05 Epitope Peptides

| SEQ ID NO. | Sequence |
|---|---|
| 2569 | DRAHYNIVTF |
| 2570 | LDLQPETTDL |
| 2571 | LRLCVQSTH |
| 2572 | IRTLEDLLM |
| 2573 | RAHYNIVTF |

In some embodiments, the donor cell source is HLA-B*35:01, and the E7 targeted T-cell subpopulation is primed and expanded with one or more E7-derived peptides selected from Table 313 (Seq. ID. Nos. 2574-2578). In some embodiments, the donor cell source is HLA-B*35:01, and the E7 targeted T-cell subpopulation is primed and expanded with E7-derived peptides selected from Table 313 (Seq. ID. Nos. 2574-2578). In some embodiments, the donor cell source is HLA-B*35:01, and the E7 targeted T-cell subpopulation is primed and expanded with E7-derived peptides comprising the peptides of Table 313 (Seq. ID. Nos. 2574-2578). In some embodiments, the donor cell source is HLA-B*35:01, and the E7 targeted T-cell subpopulation is primed and expanded with E7-derived peptides comprising the peptides of Table 313 (Seq. ID. Nos. 2574-2578) and at least one additional set of peptides based on the donor cell source HLA-B profile, wherein the at least one additional set of peptides are selected from the peptides of Tables 308-312 and 314. In some embodiments, the E7-derived peptides also include one or more sets of HLA-A and HLA-DR restricted peptides selected from Tables 301-307 and 315-320 (Seq. ID Nos. 2514-2548 and 2584-2513).

TABLE 313
HPV Strain 16 E7 HLA-B*35:01 Epitope Peptides

| SEQ ID NO. | Sequence |
|---|---|
| 2574 | QPETTDLYCY |
| 2575 | TPTLHEYML |
| 2576 | EPDRAHYNI |
| 2577 | FCCKCDSTL |
| 2578 | LEDLLMGTL |

In some embodiments, the donor cell source is HLA-B*58:02, and the E7 targeted T-cell subpopulation is primed and expanded with one or more E7-derived peptides selected from Table 314 (Seq. ID. Nos. 2579-2583). In some embodiments, the donor cell source is HLA-B*58:02, and the E7 targeted T-cell subpopulation is primed and expanded with E7-derived peptides selected from Table 314 (Seq. ID. Nos. 2579-2583). In some embodiments, the donor cell source is HLA-B*58:02, and the E7 targeted T-cell subpopulation is primed and expanded with E7-derived peptides comprising the peptides of Table 314 (Seq. ID. Nos. 2579-2583). In some embodiments, the donor cell source is HLA-B*58:02, and the E7 targeted T-cell subpopulation is primed and expanded with E7-derived peptides comprising the peptides of Table 314 (Seq. ID. Nos. 2579-2583) and at least one additional set of peptides based on the donor cell source HLA-B profile, wherein the at least one additional set of peptides are selected from the peptides of Tables 308-313. In some embodiments, the E7-derived peptides also include one or more sets of HLA-A and HLA-DR restricted peptides selected from Tables 301-307 and 315-320 (Seq. ID Nos. 2514-2548 and 2584-2513).

TABLE 314
HPV Strain 16 E7 HLA-B*58:02 Epitope Peptides

| SEQ ID NO. | Sequence |
|---|---|
| 2579 | QSTHVDIRTL |
| 2580 | RAHYNIVTF |
| 2581 | DSSEEEDEI |
| 2582 | GTLGIVCPI |
| 2583 | DTPTLHEYM |

In some embodiments, the donor cell source is HLA-DRB1*0101, and the E7 targeted T-cell subpopulation is primed and expanded with one or more E7-derived peptides selected from Table 315 (Seq. ID. Nos. 2584-2588). In some embodiments, the donor cell source is HLA-DRB1*0101, and the E7 targeted T-cell subpopulation is primed and expanded with E7-derived peptides selected from Table 315 (Seq. ID. Nos. 2584-2588). In some embodiments, the donor cell source is HLA-DRB1*0101, and the E7 targeted T-cell subpopulation is primed and expanded with E7-derived peptides comprising the peptides of Table 315 (Seq. ID. Nos. 2584-2588). In some embodiments, the donor cell source is HLA-DRB1*0101, and the E7 targeted T-cell subpopulation is primed and expanded with E7-derived peptides comprising the peptides of Table 315 (Seq. ID. Nos. 2584-2588) and at least one additional set of peptides based on the donor cell source HLA-DR profile, wherein the at least one additional set of peptides are selected from the peptides of Tables 316-320. In some embodiments, the E7-derived peptides also include one or more sets of HLA-A and HLA-B restricted peptides selected from Tables 301-314 (Seq. ID Nos. 2514-2583).

TABLE 315
HPV Strain 16 E7 HLA-DRB1*0101 Epitope Peptides

| SEQ ID NO. | Sequence |
|---|---|
| 2584 | MGTLGIVCPICSQKP |
| 2585 | HVDIRTLEDLLMGTL |
| 2586 | DLLMGTLGIVCPICS |
| 2587 | IRTLEDLLMGTLGIV |
| 2588 | TLEDLLMGTLGIVCP |

In some embodiments, the donor cell source is HLA-DRB1*0301, and the E7 targeted T-cell subpopulation is primed and expanded with one or more E7-derived peptides selected from Table 316 (Seq. ID. Nos. 2589-2593). In some embodiments, the donor cell source is HLA-DRB1*0301, and the E7 targeted T-cell subpopulation is primed and expanded with E7-derived peptides selected from Table 316 (Seq. ID. Nos. 2589-2593). In some embodiments, the donor cell source is HLA-DRB1*0301, and the E7 targeted T-cell subpopulation is primed and expanded with E7-derived peptides comprising the peptides of Table 316 (Seq. ID. Nos. 2589-2593). In some embodiments, the donor cell source is HLA-DRB1*0301, and the E7 targeted T-cell subpopulation is primed and expanded with E7-derived peptides comprising the peptides of Table 316 (Seq. ID. Nos. 2589-2593) and at least one additional set of peptides based on the donor cell source HLA-DR profile, wherein the at least one additional set of peptides are selected from the peptides of Tables 315 and 317-320. In some embodiments, the E7-derived peptides also include one or more sets of HLA-A and HLA-B restricted peptides selected from Tables 301-314 (Seq. ID Nos. 2514-2583).

TABLE 316
HPV Strain 16 E7 HLA-DRB1*0301 (DR17)
Epitope Peptides

| SEQ ID NO. | Sequence |
|---|---|
| 2589 | TFCCKCDSTLRLCVQ |
| 2590 | MLDLQPETTDLYCYE |
| 2591 | TTDLYCYEQLNDSSE |
| 2592 | IRTLEDLLMGTLGIV |
| 2593 | LHEYMLDLQPETTDL |

In some embodiments, the donor cell source is HLA-DRB1*0401, and the E7 targeted T-cell subpopulation is primed and expanded with one or more E7-derived peptides selected from Table 317 (Seq. ID. Nos. 2594-2598). In some embodiments, the donor cell source is HLA-DRB1*0401, and the E7 targeted T-cell subpopulation is primed and expanded with E7-derived peptides selected from Table 317 (Seq. ID. Nos. 2594-2598). In some embodiments, the donor cell source is HLA-DRB1*0401, and the E7 targeted T-cell subpopulation is primed and expanded with E7-derived peptides comprising the peptides of Table 317 (Seq. ID. Nos. 2594-2598). In some embodiments, the donor cell source is HLA-DRB1*0401, and the E7 targeted T-cell subpopulation is primed and expanded with E7-derived peptides comprising the peptides of Table 317 (Seq. ID. Nos. 2594-2598) and at least one additional set of peptides based on the donor cell source HLA-DR profile, wherein the at least one additional set of peptides are selected from the peptides of Tables 315-316 and 318-320. In some embodiments, the E7-derived peptides also include one or more sets of HLA-A and HLA-B restricted peptides selected from Tables 301-314 (Seq. ID Nos. 2514-2583).

TABLE 317
HPV Strain 16 E7 HLA-DRB1*0401 (DR4Dw4)
Epitope Peptides

| SEQ ID NO. | Sequence |
|---|---|
| 2594 | LHEYMLDLQPETTDL |
| 2595 | TDLYCYEQLNDSSEE |
| 2596 | RAHYNIVTFCCKCDS |
| 2597 | HEYMLDLQPETTDLY |
| 2598 | MLDLQPETTDLYCYE |

In some embodiments, the donor cell source is HLA-DRB1*0701, and the E7 targeted T-cell subpopulation is primed and expanded with one or more E7-derived peptides selected from Table 318 (Seq. ID. Nos. 2599-2603). In some embodiments, the donor cell source is HLA-DRB1*0701, and the E7 targeted T-cell subpopulation is primed and expanded with E7-derived peptides selected from Table 318 (Seq. ID. Nos. 2599-2603). In some embodiments, the donor cell source is HLA-DRB1*0701, and the E7 targeted T-cell subpopulation is primed and expanded with E7-derived peptides comprising the peptides of Table 318 (Seq. ID. Nos. 2599-2603). In some embodiments, the donor cell source is HLA-DRB1*0701, and the E7 targeted T-cell subpopulation is primed and expanded with E7-derived peptides comprising the peptides of Table 318 (Seq. ID. Nos. 2599-2603) and at least one additional set of peptides based on the donor cell source HLA-DR profile, wherein the at least one additional set of peptides are selected from the peptides of Tables 315-317 and 319-320. In some embodiments, the E7-derived peptides also include one or more sets of HLA-A and HLA-B restricted peptides selected from Tables 301-314 (Seq. ID Nos. 2514-2583).

TABLE 318
HPV Strain 16 E7 HLA-DRB1*0701 Epitope Peptides

| SEQ ID NO. | Sequence |
|---|---|
| 2599 | IVTFCCKCDSTLRLC |
| 2600 | MLDLQPETTDLYCYE |
| 2601 | THVDIRTLEDLLMGT |
| 2602 | HVDIRTLEDLLMGTL |
| 2603 | YEQLNDSSEEEDEID |

In some embodiments, the donor cell source is HLA-DRB1*1101, and the E7 targeted T-cell subpopulation is primed and expanded with one or more E7-derived peptides selected from Table 319 (Seq. ID. Nos. 2604-2608). In some embodiments, the donor cell source is HLA-DRB1*1101, and the E7 targeted T-cell subpopulation is primed and expanded with E7-derived peptides selected from Table 319 (Seq. ID. Nos. 2604-2608). In some embodiments, the donor cell source is HLA-DRB1*1101, and the E7 targeted T-cell subpopulation is primed and expanded with E7-derived peptides comprising the peptides of Table 319 (Seq. ID. Nos. 2604-2608). In some embodiments, the donor cell source is HLA-DRB1*1101, and the E7 targeted T-cell subpopulation is primed and expanded with E7-derived peptides comprising the peptides of Table 319 (Seq. ID. Nos. 2604-2608) and at least one additional set of peptides based on the donor cell source HLA-DR profile, wherein the at least one additional set of peptides are selected from the peptides of Tables 315-318 and 320. In some embodiments, the E7-derived peptides also include one or more sets of HLA-A and HLA-B restricted peptides selected from Tables 301-314 (Seq. ID Nos. 2514-2583).

TABLE 319
HPV Strain 16 E7 HLA-DRB1*1101 Epitope Peptides

| SEQ ID NO. | Sequence |
|---|---|
| 2604 | YNIVTFCCKCDSTLR |
| 2605 | DLLMGTLGIVCPICS |
| 2606 | MGTLGIVCPICSQKP |
| 2607 | TDLYCYEQLNDSSEE |
| 2608 | LYCYEQLNDSSEEED |

In some embodiments, the donor cell source is HLA-DRB1*1501, and the E7 targeted T-cell subpopulation is primed and expanded with one or more E7-derived peptides selected from Table 320 (Seq. ID. Nos. 2609-2613). In some embodiments, the donor cell source is HLA-DRB1*1501, and the E7 targeted T-cell subpopulation is primed and expanded with E7-derived peptides selected from Table 320 (Seq. ID. Nos. 2609-2613). In some embodiments, the donor cell source is HLA-DRB1*1501, and the E7 targeted T-cell subpopulation is primed and expanded with E7-derived peptides comprising the peptides of Table 320 (Seq. ID. Nos. 2609-2613). In some embodiments, the donor cell source is HLA-DRB1*1501, and the E7 targeted T-cell subpopulation is primed and expanded with E7-derived peptides comprising the peptides of Table 320 (Seq. ID. Nos. 2609-2613) and at least one additional set of peptides based on the donor cell source HLA-DR profile, wherein the at least one additional set of peptides are selected from the peptides of Tables 315-319. In some embodiments, the E7-derived peptides also include one or more sets of HLA-A and HLA-B restricted peptides selected from Tables 301-314 (Seq. ID Nos. 2514-2583).

TABLE 320
HPV Strain 16 E7 HLA-DRB1*1501 (DR2b)
Epitope Peptides

| SEQ ID NO. | Sequence |
|---|---|
| 2609 | TTDLYCYEQLNDSSE |
| 2610 | TPTLHEYMLDLQPET |
| 2611 | EDLLMGTLGIVCPIC |
| 2612 | MGTLGIVCPICSQKP |
| 2613 | HYNIVTFCCKCDSTL |

Ratio of T-Cell Subpopulations in MUSTANG Compositions

The MUSTANG composition of the present invention is comprised of two or more T-cell subpopulations each targeting a single TAA. The T-cell subpopulations used to create the MUSTANG composition can be combined in a single dosage form for administration, or each administered separately, wherein the separate T-cell subpopulations collectively comprise the MUSTANG composition. In some embodiments, the MUSTANG composition comprises T-cell subpopulations in a ratio or percentage reflective or correlative of the relative identified TAA expression profile of the patient. In some embodiments, the T-cell subpopulations used to create the MUSTANG composition are in about an equal ratio. In some embodiments, the MUSTANG composition comprises two or more T-cell subpopulations, wherein each T-cell subpopulation is specific for a different TAA.

The ratios of the T-cell subpopulations in the MUSTANG composition may be selected based on the knowledge of the patient's tumor characteristics or the healthcare provider's best judgement. In some embodiments, the composition comprises two T-cell subpopulations, wherein the MUSTANG comprises (i) at least about 45%, 50%, 55%, 60%, 65%, 70%, 75%, 80%, or 85% of a first T-cell subpopulation and (ii) at least about 15%, 20%, 25%, 30%, 35%, 40%, 45%, 50%, or 55% of a second T-cell subpopulation, wherein the percentage adds to 100% by weight.

In some embodiments, the ratio or percentage of each T-cell subpopulation is normalized based on the measured activity of each T-cell subpopulation against the TAA as measured by, for example, but not limited to, the EliSpot assay. The activity data generated for each T-cell subpopulation can be used to ensure that the activity of each T-cell subpopulation is approximately equal or normalized. As shown in FIG. 10, the PRAMS activity of the MUSTANG composition described in Example 5 is approximately 10-fold greater than the activity of WT1 and Survivin. To control for the level of activity in the MUSTANG composition differing amounts of the T-cell subpopulations can be combined to create a MUSTANG composition that has roughly equal activity across all antigens if desired, or also used to create variable ratios based on the expression profile of the patient's tumor. In order to generate a MUSTANG composition that has equal activity for all three antigens it would be necessary to combine the WT1, Survivin, and PRAME T-cell subpopulations in a 10:10:1 ratio. This ratio can be adjusted based on the desired activity of each T-cell subpopulation in the final MUSTANG composition. In some embodiments, the percentage of the first and second T-cell subpopulations is based on the TAA expression profile of a malignancy or tumor such that the percentage of the first and second T-cell subpopulations correlates with the TAA expression profile of the tumor.

The MUSTANG composition can include two, three, four, five, or more T-cell subpopulations. The T-cell subpopulations can be included in the MUSTANG composition in about an equal ratio, or in a ratio that reflects the individual TAA expression as determined by the patient's TAA expression profile, or in an alternative ratio. In an alternative embodiment, the T-cell subpopulations can be included in a ratio that reflects a greater percentage of T-cell subpopulations directed to known TAAs which show high immunogenicity.

In a particular embodiment, the MUSTANG composition comprises at least two T-cell subpopulations, wherein the first T-cell subpopulation is specific to PRAME and the second T-cell subpopulation is selected from the group consisting of WT1, survivin, NY-ESO-1 and MAGE-A3.

In a particular embodiment, the MUSTANG composition comprises at least two T-cell subpopulations, wherein the first T-cell subpopulation to survivin and the second T-cell subpopulation is selected from the group consisting of WT1, NY-ESO-1 and MAGE-A3.

In a particular embodiment, the MUSTANG composition comprises at least two T-cell subpopulations, wherein the first T-cell subpopulation is specific to WT1 and the second T-cell subpopulation is selected from the group consisting of NY-ESO-1 and MAGE-A3.

In a particular embodiment, the MUSTANG composition comprises at least two T-cell subpopulations, wherein the first T-cell subpopulation is specific to NY-ESO-1 and the second T-cell subpopulation is specific to MAGE-A3.

In some embodiments, the MUSTANG composition comprises a first T-cell subpopulation, a second T-cell subpopulation, and a third T-cell subpopulation, wherein each T-cell subpopulation is specific for a different TAA. In some embodiments, the T-cell subpopulations used to create the MUSTANG composition are in about an equal ratio.

The ratios of the T-cell subpopulations in the MUSTANG composition may be selected based on the knowledge of the patient's tumor characteristics or the healthcare provider's best judgement. In some embodiments, the composition comprises three T-cell subpopulations, wherein the MUSTANG composition comprises (i) at least about 45%, 50%, 55%, 60%, 65%, 70%, 75%, 80%, or 85% of the first T-cell subpopulation, (ii) at least about 5%, 10%, 15%, 20%, or 25% of the second T-cell subpopulation and (iii) at least about 10%, 15%, 20%, 25%, 30%, or 35% of the third T-cell subpopulation, wherein the percentage adds to 100% by weight. In some embodiments, the percentage of the T-cell subpopulations is based on the TAA expression profile of a malignancy or tumor such that the percentage of the first, second, and third T-cell subpopulations correlates with the TAA expression profile of the tumor. In some embodiments, the ratio or percentage of each T-cell subpopulation is normalized based on the measured activity of each T-cell subpopulation against the TAA as measured by, for example, but not limited to, the Eli Spot assay.

In some embodiments, the TAA is selected from survivin, MAGE-A3, NY-ESO-1, PRAME, and WT1.

In a particular embodiment, the MUSTANG composition comprises at least three T-cell subpopulations, wherein the first T-cell subpopulation is specific to PRAME, the second T-cell subpopulation is specific to WT1, and the third T-cell subpopulation is selected from the group consisting of survivin, NY-ESO-1 and MAGE-A3.

In another particular embodiment, the MUSTANG composition comprises at least three T-cell subpopulations, wherein the first T-cell subpopulation is specific to PRAME, the second T-cell subpopulation is specific to NY-ESO-1, and the third T-cell subpopulation is specific to MAGE-A3.

In another particular embodiment, the MUSTANG composition comprises at least three T-cell subpopulations, wherein the first T-cell subpopulation composition is specific to WT1, the second T-cell subpopulation is specific to NY-ESO-1, and the third T-cell subpopulation is specific to MAGE-A3.

In some embodiments, the MUSTANG composition comprises a first T-cell subpopulation, a second T-cell subpopulation, a third T-cell subpopulation, and a fourth T-cell subpopulation, wherein each T-cell subpopulation is specific for a different TAA. In some embodiments, the T-cell subpopulations used to create the MUSTANG composition are in about an equal ratio.

The ratios of the T-cell subpopulations in the MUSTANG composition may be selected based on the knowledge of the patient's tumor characteristics or the healthcare provider's best judgement. In some embodiments, the composition comprises four T-cell subpopulations, wherein the MUSTANG composition comprises (i) at least about 45%, 50%, 55%, 60%, 65%, 70%, 75%, 80%, or 85% of the first T-cell subpopulation, (ii) at least about 5%, 10%, 15%, 20%, 25%, 30%, 35%, 40% or 45% of the second T-cell subpopulation, (iii) at least about 5%, 10%, 15%, 20%, 25%, 30%, 35%, 40% or 45% of the third T-cell subpopulation, and (iv) at least about 5%, 10%, 15%, 20%, 25%, 30%, 35%, 40% or 45% of the fourth T-cell subpopulation, wherein the percentage adds to 100% by weight. In some embodiments, the ratio or percentage of each T-cell subpopulation is normalized based on the measured activity of each T-cell subpopulation against the TAA as measured by, for example, but not limited to, the EliSpot assay. In some embodiments, the percentage of the T-cell subpopulations is based on the TAA expression profile of a malignancy or tumor such that the percentage of the first, second, third and fourth T-cell subpopulations correlates with the TAA expression profile of the tumor. In some embodiments, the T-cell subpopulations are specific to a TAA selected from survivin, MAGE-A3, NY-ESO-1, PRAME, and WT1.

In a particular embodiment, the MUSTANG composition comprises at least four T-cell subpopulations, wherein the first T-cell subpopulation is specific to PRAME, the second T-cell subpopulation is specific to WT1, the third T-cell subpopulation is specific to survivin and the fourth T-cell subpopulation is selected from the group consisting of MAGE-A3 and NY-ESO-1.

In a further embodiment, the MUSTANG composition comprises at least four T-cell subpopulations, wherein the first T-cell subpopulation is specific to PRAME, the second T-cell subpopulation is specific to WT1, the third T-cell subpopulation is specific to NY-ESO-1 and the fourth T-cell subpopulation is specific to MAGE-A3.

In a still further embodiment, the MUSTANG composition comprises at least four T-cell subpopulations, wherein the first T-cell subpopulation is specific to PRAME, the second T-cell subpopulation is specific to survivin, the third T-cell subpopulation is specific to NY-ESO-1, and the fourth T-cell subpopulation is specific to MAGE-A3.

In some embodiments, the MUSTANG composition comprises a first T-cell subpopulation, a second T-cell subpopulation, a third T-cell subpopulation, a fourth T-cell subpopulation, and a fifth T-cell subpopulation, wherein each T-cell subpopulation is specific for a different tumor-associated antigen. In some embodiments, the T-cell subpopulations used to create the MUSTANG composition are in about an equal ratio.

The ratios of the T-cell subpopulations in the MUSTANG composition may be selected based on the knowledge of the patient's tumor characteristics or the healthcare provider's best judgement. In some embodiments, the composition comprises five T-cell subpopulations, wherein the MUSTANG composition comprises (i) at least about 45%, 50%, 55%, 60%, 65%, 70%, 75%, 80% of the first T-cell subpopulation, (ii) at least about 5%, 10%, 15%, 20%, 25%, 30%, 35%, or 40% of the second T-cell subpopulation, (iii) at least about 5%, 10%, 15%, 20%, 25%, 30%, 35%, or 40% of the third T-cell subpopulation, (iv) at least about 5%, 10%, 15%, 20%, 25%, 30%, 35%, or 40% of the fourth T-cell subpopulation and (v) at least about 5%, 10%, 15%, 20%, 25%, 30%, 35%, or 40% of the fifth T-cell subpopulation, wherein the percentage adds to 100% by weight. In some embodiments, the ratio or percentage of each T-cell subpopulation is normalized based on the measured activity of each T-cell subpopulation against the TAA as measured by, for example, but not limited to, the Eli Spot assay. In some embodiments, the percentage of the T-cell subpopulations is based on the TAA expression profile of a malignancy or tumor such that the percentage of the first, second, third, fourth and fifth T-cell subpopulations correlates with the TAA expression profile of the tumor. In some embodiments, each of the five T-cell subpopulations are specific to survivin, MAGE-A3, NY-ESO-1, PRAME, and WT1, respectively.

In some embodiments, the MUSTANG composition comprises at least five T-cell subpopulations, wherein the first T-cell subpopulation is specific to PRAME, the second T-cell subpopulation is specific to WT1, the third T-cell subpopulation is specific to survivin, the fourth T-cell subpopulation is specific to MAGE-A3 and the fifth T-cell subpopulation is specific to NY-ESO-1.

In some embodiments, the mononuclear cell sample from which the T-cell subpopulations are isolated is derived from the human to which the composition is also administered (autologous).

In some embodiments, the mononuclear cell sample from which the T-cell subpopulations are isolated is derived from a cell donor (allogeneic). In certain embodiments, the allogeneic T-cell subpopulation composition has at least one HLA allele or HLA allele combination in common with the patient. In certain embodiments, the allogeneic T-cell subpopulation composition has more than one HLA allele or HLA allele combination in common with the patient. In certain embodiments, the tumor-associated antigen activity of the MUSTANG composition is through at least one HLA allele or HLA allele combination in common with the patient. In certain embodiments, the allogeneic T-cell subpopulations comprising the MUSTANG composition are recognized through the same shared HLA restriction. In certain embodiments, the allogeneic T-cell subpopulations comprising the MUSTANG composition are recognized through different shared HLA restrictions.

In a second aspect, the present invention provides a method of treating a disease or disorder comprising administering an effective amount of the MUSTANG composition disclosed herein to a patient, typically a human in need thereof.

In some embodiments, the method further comprises isolating a mononuclear cell sample from the patient, typically a human to which the MUSTANG composition is administered (autologous), wherein the MUSTANG composition comprises T-cell subpopulations made from the mononuclear cell sample.

In some embodiments, the method further comprises isolating a mononuclear cell sample from a cell donor (allogeneic), wherein the MUSTANG composition comprises T-cell subpopulations made from the mononuclear cell sample. In certain embodiments, the allogeneic MUSTANG composition has at least one HLA allele or HLA allele combination in common with the patient. In certain embodiments, the allogeneic MUSTANG composition has more than one HLA allele or HLA allele combination in common with the patient. In certain embodiments, the TAA activity of the MUSTANG composition is through at least one HLA allele or HLA allele combination in common with the patient. In certain embodiments, the TAA activity of the MUSTANG composition is through more than one HLA allele or HLA allele combination in common with the patient. In certain embodiments, the allogeneic T-cell subpopulations comprising the MUSTANG composition are recognized through the same shared HLA restriction. In certain embodiments, the allogeneic T-cell subpopulations comprising the MUSTANG composition are recognized through different shared HLA restrictions. In certain embodiments the MUSTANG composition selected has the most shared HLA alleles or allele combinations and the highest TAA specificity.

In certain embodiments, the method further comprises selecting the MUSTANG composition based on the TAA expression profile of the malignancy or tumor of the patient.

In certain embodiments, the method further comprises selecting the MUSTANG composition based on the levels of circulating TAA-specific T-cells present in the patient after administration of a MUSTANG composition. Methods of measuring the levels of circulating TAA-specific T-cells present in the patient are known in the art and non-limiting exemplary methods include Elispot assay, TCR sequencing, intracellular cytokine staining, and through the uses of WIC-peptide multimers.

Method of Treating a Patient with a Tumor by Administering a MUSTANG Composition

The invention includes a method to treat a patient with a tumor, typically a human, by administering an effective amount of a MUSTANG composition described herein.

The dose administered may vary. In some embodiments, the MUSTANG composition is administered to a patient, such as a human in a dose ranging from 1×10⁶ cells/m² to 1×10⁸ cells/m². The dose can be a single dose, for example, comprising the combination of all of the T-cell subpopulations comprising the MUSTANG composition, or multiple separate doses, wherein each dose comprises a separate T-cell subpopulation and the collective separate doses of T-cell subpopulations comprise the total MUSTANG composition. In some embodiments, the MUSTANG composition dosage is 1×10⁶ cells/m², 2×10⁶ cells/m², 3×10⁶ cells/m², 4×10⁶ cells/m², 5×10⁶ cells/m², 6×10⁶ cells/m², 7×10⁶ cells/m², 8×10⁶ cells/m², 9×10⁶ cells/m², 1×10⁷ cells/m², 2×10⁷ cells/m², 3×10⁷ cells/m², 4×10⁷ cells/m², 5×10⁷ cells/m², 6×10⁷ cells/m²,7×10⁷ cells/m², 8×10⁷ cells/m², 9×10⁷ cells/m², or 1×10⁸ cells/m².

The MUSTANG composition may be administered by any suitable method. In some embodiments, the MUSTANG composition is administered to a patient, such as a human as an infusion and in a particular embodiment, an infusion with a total volume of 1 to 10 cc. In some embodiments, the MUSTANG composition is administered to a patient as a 1, 2, 3, 4, 5, 6, 7, 8, 9, or 10 cc infusion. In some embodiments, the MUSTANG composition when present as an infusion is administered to a patient over 10, 20, 30, 40, 50, 60 or more minutes to the patient in need thereof.

In some embodiments, a patient receiving an infusion has vital signs monitored before, during, and 1-hour post infusion of the MUSTANG composition. In certain embodiments, patients with stable disease (SD), partial response (PR), or complete response (CR) up to 6 weeks after initial infusion may be eligible to receive additional infusions, for example, 1, 2, 3, 4, 5, 6, 7, 8, 9 or 10 additional infusions several weeks apart, for example, up to about 2, 3, 4, 5, 6, 7, 8, 9 or 10 weeks apart.

### Determining the Tumor-Associated Antigen Expression Profile

Determining a TAA expression profile can be performed by any method known in the art. Non-limiting exemplary methods for determining a tumor-associated antigen expression profile can be found in Ding et al., Cancer Bio Med (2012) 9: 73-76; Qin et al., Leukemia Research (2009) 33(3) 384-390; and Weber et al., Leukemia (2009) 23: 1634-1642. In some embodiments, TAA expression profiles are generated from a sample collected from a patient with a malignancy or tumor. In some embodiments, the sample is selected from a group consisting of blood, bone marrow, and tumor biopsy.

In some embodiments, the TAA expression profile is determined from a blood sample of a patient with a malignancy or tumor. In some embodiments, the TAA expression profile is determined from a bone marrow sample of a patient with a malignancy or tumor. In some embodiments, the TAA expression profile is determined from a tumor biopsy sample of a patient with a malignancy or tumor.

In some embodiments, genetic material is extracted from the sample collected from a patient with a malignancy or tumor. In some embodiments, the genetic material is selected from a group consisting of total RNA, messenger RNA and genomic DNA.

After extraction of genetic material, quantitative reverse transcriptase polymerase chain reaction (qPCR) is performed on the genetic material utilizing primers developed from TAAs of interest.

The patient's tumor cells can be checked for reactivity against activated T-cell subpopulations and/or the MUSTANG composition of the present invention using any known methods, including cytotoxicity assays described herein.

### Determining the Levels of Circulating TAA-Specific T-Cells

Determining the levels of circulating TAA-specific T-cells after infusion of the MUSTANG composition can be performed by any method known in the art. Non-limiting exemplary methods for determining levels of circulating TAA-specific T-cells include Elispot assay, intracellular cytokine staining, multimer analysis, and TCR sequencing and can be found in Chapuis et al., Sci Transl Med (2013) 5(174): 174ra27 and Hanley et al., Sci Transl Med (2015) 7(285): 285ra63, which are incorporated herein by reference. In some embodiments, levels of circulating TAA-specific T-cells is determined from a sample collected from a patient with a malignancy or tumor treated with a MUSTANG composition. In some embodiments, the sample is selected from a group consisting of blood, peripheral blood mononuclear cells, and bone marrow.

In some embodiments, the levels of circulating TAA-specific T-cells is determined from a blood sample of a patient with a malignancy or tumor treated with a MUSTANG composition. In some embodiments, the levels of circulating TAA-specific T-cells is determined from a peripheral blood mononuclear cell sample of a patient with a malignancy or tumor treated with a MUSTANG composition. In some embodiments, the levels of circulating TAA-specific T-cells is determined from a bone marrow sample of a patient with a malignancy or tumor treated with a MUSTANG composition.

In some embodiments, the levels of circulating TAA-specific T-cells is determined using an Elispot assay. In some embodiments, the levels of circulating TAA-specific T-cells is determined using an intracellular cytokine staining assay. In some embodiments, the levels of circulating TAA-specific T-cells is determined using multimer analysis. In some embodiments, the levels of circulating TAA-specific T-cells is determined by TCR sequencing.

### Hematological and Solid Tumors Targeted for Treatment

The MUSTANG compositions described herein can be used to treat a patient with a solid or hematological tumor.

Lymphoid neoplasms are broadly categorized into precursor lymphoid neoplasms and mature T-cell, B-cell or natural killer cell (NK) neoplasms. Chronic leukemias are those likely to exhibit primary manifestations in blood and bone marrow, whereas lymphomas are typically found in extramedullary sites, with secondary events in the blood or bone. Over 79,000 new cases of lymphoma were estimated in 2013. Lymphoma is a cancer of lymphocytes, which are a type of white blood cell. Lymphomas are categorized as Hodgkin's or non-Hodgkin's. Over 48,000 new cases of leukemias were expected in 2013.

In some embodiments, the disease or disorder is a hematological malignancy selected from a group consisting of leukemia, lymphoma and multiple myeloma.

In some embodiments, the methods described herein can be used to treat a leukemia. For example, the patient such as a human may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T-cell chronic leukemia. In some embodiments, the patient suffers from an acute myelogenous leukemia, for example an undifferentiated AML (M0); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

In a particular embodiment, the hematological malignancy is a lymphoma or lymphocytic or myelocytic proliferation disorder or abnormality. In some embodiments, the lymphoma is a non-Hodgkin's lymphoma. In some embodiments, the lymphoma is a Hodgkin's lymphoma.

In some aspects, the methods described herein can be used to treat a patient such as a human, with a Non-Hodgkin's Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia.

Alternatively, the methods described herein can be used to treat a patient, such as a human, with a Hodgkin's Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

Alternatively, the methods described herein can be used to treat a patient, for example a human, with specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma.

Abnormal proliferation of T-cells, B-cells, and/or NK-cells can result in a wide range of cancers. A host, for example a human, afflicted with any of these disorders can be treated with an effective amount of the TAA-L composition as described herein to achieve a decrease in symptoms (a palliative agent) or a decrease in the underlying disease (a disease modifying agent).

Alternatively, the methods described herein can be used to treat a patient, such as a human, with a hematological malignancy, for example but not limited to T-cell or NK-cell lymphoma, for example, but not limited to: peripheral T-cell lymphoma; anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In some embodiments, the MUSTANG composition disclosed herein is used to treat a patient with a selected hematopoietic malignancy either before or after hematopoietic stem cell transplantation (HSCT). In some embodiments, the MUSTANG composition is used to treat a patient with a selected hematopoietic malignancy after HSCT. In some embodiments, the MUSTANG composition is used to treat a patient with a selected hematopoietic malignancy up to about 30, 35, 40, 45, or 50 days after HSCT. In some embodiments, the MUSTANG composition is used to treat a patient with a selected hematopoietic malignancy after neutrophil engraftment during the period following HSCT. In some embodiments, the MUSTANG composition is used to treat a patient with a selected hematopoietic malignancy before HSCT, such as one week, two weeks, three weeks or more before HSCT.

In some aspects, the tumor is a solid tumor. In some embodiments, the solid tumor is Wilms Tumor. In some embodiments, the solid tumor is osteosarcoma. In some embodiments, the solid tumor is Ewing sarcoma. In some embodiments, the solid tumor is neuroblastoma. In some embodiments, the solid tumor is soft tissue sarcoma. In some embodiments, the solid tumor is rhabdomyosarcoma.

Non-limiting examples of tumors that can be treated according to the present invention include, but are not limited to, acoustic neuroma, adenocarcinoma, adrenal gland cancer, anal cancer, angiosarcoma (e.g., lymphangiosarcoma, lymphangioendotheli osarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast, triple negative breast cancer, HER2-negative breast cancer, HER2-positive breast cancer, male breast cancer, late-line metastatic breast cancer, progesterone receptor-negative breast cancer, progesterone receptor-positive breast cancer, recurrent breast cancer), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), glioblastoma multiforme, head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

### Administration of MUSTANG Compositions

Methods for administration of cells for adoptive cell therapy are known and may be used in connection with the provided methods and MUSTANG compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The administration of the MUSTANG composition may vary. In one aspect, the MUSTANG composition may be administered to a patient such as a human at an interval selected from once every 1 week, 2 weeks, 3 weeks, 4 weeks, 5 weeks, 6 weeks, 7 weeks, 8 weeks, or more after the initial administration of the MUSTANG composition. In a typical embodiment, the MUSTANG composition is administered in an initial dose then at every 4 weeks thereafter. In some embodiments, the MUSTANG composition may be administered repetitively to 1, 2, 3, 4, 5, 6, or more times after the initial administration of the composition. In a typical embodiment, the MUSTANG composition is administered repetitively up to 10 more times after the initial administration of the MUSTANG composition. In an alternative embodiment, the MUSTANG composition is administered more than 10 times after the initial administration of the MUSTANG composition.

In some embodiments, a TAA expression profile of the malignancy or tumor of the patient, for example, a human is performed prior to the initial administration of the MUSTANG composition. In some embodiments, a TAA expression profile of the malignancy or tumor of the patient is performed prior to each subsequent administration of the MUSTANG composition, allowing for the option to adjust the MUSTANG composition. In some embodiments, the MUSTANG composition of subsequent administrations remains the same as the initial administration. In some embodiments, the MUSTANG composition of subsequent administrations is changed based on the change in the TAA expression profile of the malignancy or tumor of the patient.

In some embodiments, the MUSTANG compositions are administered to a subject in the form of a pharmaceutical composition, such as a composition comprising the cells or cell populations and a pharmaceutically acceptable carrier or excipient. The pharmaceutical compositions in some embodiments additionally comprise other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The choice of carrier in the pharmaceutical composition may be determined in part by the by the particular method used to administer the cell composition. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

In addition, buffering agents in some aspects are included in the composition. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins 21st ed. (May 1, 2005).

In some embodiments, the pharmaceutical composition comprises the MUSTANG composition in an amount that is effective to treat or prevent the disease or condition, such as a therapeutically effective or prophylactically effective amount. Thus, in some embodiments, the methods of administration include administration of the MUSTANG composition at effective amounts. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the MUSTANG composition is administered at a desired dosage, which in some aspects includes a desired dose or number of cells and/or a desired ratio of T-cell subpopulations. Thus, the dosage of cells in some embodiments is based on a total number of cells (or number per m² body surface area or per kg body weight) and a desired ratio of the individual populations or sub-types. In some embodiments, the dosage of cells is based on a desired total number (or number per m² body surface area or per kg of body weight) of cells in the individual populations or of individual cell types. In some embodiments, the dosage is based on a combination of such features, such as a desired number of total cells, desired ratio, and desired total number of cells in the individual populations.

In some embodiments, the MUSTANG composition is administered at or within a tolerated difference of a desired dose of total cells, such as a desired dose of T cells. In some aspects, the desired dose is a desired number of cells, a desired number of cells per unit of body surface area or a desired number of cells per unit of body weight of the subject to whom the cells are administered, e.g., cells/m² or cells/kg. In some aspects, the desired dose is at or above a minimum number of cells or minimum number of cells per unit of body surface area or body weight. In some aspects, among the total cells, administered at the desired dose, the individual populations or sub-types are present at or near a desired output ratio as described herein, e.g., within a certain tolerated difference or error of such a ratio.

In some embodiments, the cells are administered at or within a tolerated difference of a desired dose. In some aspects, the desired dose is a desired number of cells, or a desired number of such cells per unit of body surface area or body weight of the subject to whom the cells are administered, e.g., cells/m² or cells/kg. In some aspects, the desired dose is at or above a minimum number of cells of the population, or minimum number of cells of the population per unit of body surface area or body weight.

Thus, in some embodiments, the dosage is based on a desired fixed dose of total cells and a desired ratio, and/or based on a desired fixed dose of two or more, e.g., each, of the individual T-cell subpopulations. Thus, in some embodiments, the dosage is based on a desired fixed or minimum dose of T-cell subpopulations and a desired ratio thereof.

In certain embodiments, MUSTANG composition is administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual T-cell subpopulations of cells is within a range of between at or about 10⁴ and at or about 10⁹ cells/meter² (m²) body surface area, such as between 10⁵ and 10⁶ cells/m² body surface area, for example, at or about 1×10⁵ cells/m², 1.5×10⁵ cells/m², 2×10⁵ cells/m², or 1×10⁶ cells/m² body surface area. For example, in some embodiments, the cells are administered at, or within a certain range of error of, between at or about 10⁴ and at or about 10⁹ T cells/meter² (m²) body surface area, such as between 10⁵ and 10⁶ T cells/m² body surface area, for example, at or about 1×10⁵ T cells/m², 1.5×10⁵ T cells/m², 2×10⁵ T cells/m², or 1×10⁶ T cells/m² body surface area.

In some embodiments, the cells are administered at or within a certain range of error of between at or about 10⁴ and at or about 10⁹ cells/meter² (m²) body weight, such as between 10⁵ and 10⁶ cells/m² body weight, for example, at or about 1×10⁵ cells/m², 1.5×10⁵ cells/m², 2×10⁵ cells/kg, or 1×10⁶ cells/m² body surface area.

### Product Release Testing and Characterization of T-Cell Subpopulations

Prior to infusion, the MUSTANG composition may be characterized for safety and release testing. Product release testing, also known as lot or batch release testing, is an important step in the quality control process of drug substances and drug products. This testing verifies that a T-cell subpopulation and/or MUSTANG composition meets a pre-determined set of specifications. Pre-determined release specifications for T-cell subpopulations and MUSTANG compositions include confirmation that the cell product is >70% viable, has <5.0 EU/ml of endotoxin, is negative for aerobic, anaerobic, fungal pathogens and mycoplasma, and lacks reactivity to allogeneic PHA blasts, for example, with less than 10% lysis to PHA blasts. The phenotype of the MUSTANG composition may be determined with requirements for clearance to contain, in one non-limiting embodiment, <2% dendritic cells and <2% B cells. The HLA identity between the MUSTANG composition and the donor is also confirmed.

Antigen specificity of the T-cell subpopulations can be tested via an Interferon-y Enzyme-Linked Immunospot (IFNγ ELISpot) assay. Other cytokines can also be utilized to measure antigen specificity including TNFα and IL-4. Pre-stimulated effector cells and target cells pulsed with the TAA of interest are incubated in a 96-well plate (pre-incubated with anti-INF-γ antibody) at an E/T ratio of 1:2. They are compared with no-TAA control, an irrelevant peptide not used for T-cell generation, and SEB as a positive control. After washing, the plates are incubated with a biotinylated anti-IFN-γ antibody. Spots are detected by incubating with streptavidin-coupled alkaline phosphastase and substrate. Spot forming cells (SFCs) are counted and evaluated using an automated plate reader.

The phenotype of the MUSTANG composition can be determined by extracellular antibody staining with anti-CD3, CD4, CD8, CD45, CD19, CD16, CD56, CD14, CD45, CD83, HLA-DR, TCRαβ, TCRγδ and analyzed on a flow cytometer. Annexin-V and PI antibodies can be used as viability controls, and data analyzed with FlowJo Flow Cytometry software (Treestar, Ashland, Oreg., USA).

The lytic capacity of T-cell subpopulations can be evaluated via ⁵¹Chromium (⁵¹Cr) and Europium (Eu)-release cytotoxicity assays to test recognition and lysis of target cells by the T-cell subpopulations and MUSTANG compositions.

Typically, activated primed T-cells (effector cells) can be tested against ⁵¹Cr-labeled target cells at effector-to-target ratios of, for example, 40:1, 20:1, 10:1, and 5:1. Cytolytic activity can be determined by measuring ⁵¹Cr release into the supernatant on a gamma-counter. Spontaneous release is assessed by incubating target cells alone, and maximum lysis by adding 1% Triton X-100. Specific lysis was calculated as: specific lysis (%)=(experimental release−spontaneous release)/(maximum release−spontaneous release)×100.

Europium-release assays can also be utilized to measure the lytic capacity of T-cell subpopulations and MUSTANG compositions. This is a non-radioactive alternative to the conventional Chromium-51 (⁵¹Cr) release assay and works on the same principle as the radioactive assay. Target cells are first loaded with an acetoxymethyl ester of BATDA. The ligand penetrates the cell membrane quickly. Within the cell, the ester bonds are hydrolyzed to form a hydrophilic ligand (TDA), which no longer passes through the cell membrane. If cells are lysed by an effector cell, TDA is released outside the cell into the supernatant. Upon addition of Europium solution to the supernatant, Europium can form a highly fluorescent and stable chelate with the released TDA (EuTDA). The measured fluorescence signal correlates directly with the number of lysed cells in the cytotoxicity assay. Specific lysis was calculated as: specific lysis (%)=(experimental release−spontaneous release)/(maximum release−spontaneous release)×100.

### Monitoring

Following administration of the cells, the biological activity of the administered cell populations in some embodiments is measured, e.g., by any of a number of known methods. Parameters to assess include specific binding of a T-cell or other immune cell to antigen, in vivo, e.g., by imaging, or ex vivo, e.g., by ELISA or flow cytometry. In certain embodiments, the ability of the administered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004), all incorporated herein by reference. In certain embodiments, the biological activity of the cells is measured by assaying expression and/or secretion of one or more cytokines, such as IFNγ, IL-2, and TNF. In some aspects the biological activity is measured by assessing clinical outcome, such as reduction in tumor burden or load.

### Combination Therapies

In one aspect of the invention, MUSTANG compositions disclosed herein can be beneficially administered in combination with another therapeutic regimen for beneficial, additive, or synergistic effects.

In some embodiments, the MUSTANG composition is administered in combination with another therapy to treat a hematological malignancy. In some embodiments, the MUSTANG composition is administered in combination with another therapy to treat a solid tumor. The second therapy can be a pharmaceutical or a biologic agent (for example an antibody) to increase the efficacy of treatment with a combined or synergistic approach.

In some embodiments, the additional therapy is a monoclonal antibody (MAb). Some MAbs stimulate an immune response that destroys tumor cells. Similar to the antibodies produced naturally by B cells, these MAbs “coat” the tumor cell surface, triggering its destruction by the immune system. FDA-approved MAbs of this type include rituximab, which targets the CD20 antigen found on non-Hodgkin lymphoma cells, and alemtuzumab, which targets the CD52 antigen found on B-cell chronic lymphocyticleukemia (CLL) cells. Rituximab may also trigger cell death (apoptosis) directly. Another group of MAbs stimulates an antitumor immune response by binding to receptors on the surface of immune cells and inhibiting signals that prevent immune cells from attacking the body's own tissues, including tumor cells. Other MAbs interfere with the action of proteins that are necessary for tumor growth. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR). MAbs that bind to cell surface growth factor receptors prevent the targeted receptors from sending their normal growth-promoting signals. They may also trigger apoptosis and activate the immune system to destroy tumor cells. Another group of tumor therapeutic MAbs are the immunoconjugates. These MAbs, which are sometimes called immunotoxins or antibody-drug conjugates, consist of an antibody attached to a cell-killing substance, such as a plant or bacterial toxin, a chemotherapy drug, or a radioactive molecule. The antibody latches onto its specific antigen on the surface of a tumor cell, and the cell-killing substance is taken up by the cell. FDA-approved conjugated MAbs that work this way include 90Y-ibritumomab tiuxetan, which targets the CD20 antigen to deliver radioactive yttrium-90 to B-cell non-Hodgkin lymphoma cells; ¹³¹I-tositumomab, which targets the CD20 antigen to deliver radioactive ¹³¹I to non-Hodgkin lymphoma cells.

In some embodiments, the additional agent is an immune checkpoint inhibitor (ICI), for example, but not limited to PD-1 inhibitors, PD-L1 inhibitors, PD-L2 inhibitors, CTLA-4 inhibitors, LAG-3 inhibitors, TIM-3 inhibitors, and V-domain Ig suppressor of T-cell activation (VISTA) inhibitors, or combinations thereof.

In some embodiments, the immune checkpoint inhibitor is a PD-1 inhibitor that blocks the interaction of PD-1 and PD-L1 by binding to the PD-1 receptor, and in turn inhibits immune suppression. In some embodiments, the immune checkpoint inhibitor is a PD-1 immune checkpoint inhibitor selected from nivolumab (Opdivo®), pembrolizumab (Keytruda®), pidilizumab, AMP-224 (AstraZeneca and MedImmune), PF-06801591 (Pfizer), MEDI0680 (AstraZeneca), PDR001 (Novartis), REGN2810 (Regeneron), MGA012 (MacroGenics), BGB-A317 (BeiGene) SHR-12-1 (Jiangsu Hengrui Medicine Company and Incyte Corporation), TSR-042 (Tesaro), and the PD-L1/VISTA inhibitor CA-170 (Curis Inc.).

In some embodiments, the immune checkpoint inhibitor is the PD-1 immune checkpoint inhibitor nivolumab (Opdivo®) administered in an effective amount for the treatment of Hodgkin's lymphoma. In another aspect of this embodiment, the immune checkpoint inhibitor is the PD-1 immune checkpoint inhibitor pembrolizumab (Keytruda®) administered in an effective amount. In an additional aspect of this embodiment, the immune checkpoint inhibitor is the PD-1 immune checkpoint inhibitor pidilizumab (Medivation) administered in an effective amount for refractory diffuse large B-cell lymphoma (DLBCL).

In some embodiments, the immune checkpoint inhibitor is a PD-L1 inhibitor that blocks the interaction of PD-1 and PD-L1 by binding to the PD-L1 receptor, and in turn inhibits immune suppression. PD-L1 inhibitors include, but are not limited to, atezolizumab, durvalumab, KNO35CA-170 (Curis Inc.), and LY3300054 (Eli Lilly).

In some embodiments, the immune checkpoint inhibitor is the PD-L1 immune checkpoint inhibitor atezolizumab (Tecentriq®) administered in an effective amount. In another aspect of this embodiment, the immune checkpoint inhibitor is durvalumab (AstraZeneca and Medlmmune) administered in an effective. In yet another aspect of the embodiment, the immune checkpoint inhibitor is KN035 (Alphamab). An additional example of a PD-L1 immune checkpoint inhibitor is BMS-936559 (Bristol-Myers Squibb), although clinical trials with this inhibitor have been suspended as of 2015.

In one aspect of this embodiment, the immune checkpoint inhibitor is a CTLA-4 immune checkpoint inhibitor that binds to CTLA-4 and inhibits immune suppression. CTLA-4 inhibitors include, but are not limited to, ipilimumab, tremelimumab (AstraZeneca and MedImmune), AGEN1884 and AGEN2041 (Agenus).

In some embodiments, the CTLA-4 immune checkpoint inhibitor is ipilimumab (Yervoy®) administered in an effective amount

In another embodiment, the immune checkpoint inhibitor is a LAG-3 immune checkpoint inhibitor. Examples of LAG-3 immune checkpoint inhibitors include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). In yet another aspect of this embodiment, the immune checkpoint inhibitor is a TIM-3 immune checkpoint inhibitor. A specific TIM-3 inhibitor includes, but is not limited to, TSR-022 (Tesaro).

Other immune checkpoint inhibitors for use in combination with the invention described herein include, but are not limited to, B7-H3/CD276 immune checkpoint inhibitors such as MGA217, indoleamine 2,3-dioxygenase (IDO) immune checkpoint inhibitors such as Indoximod and INCB024360, killer immunoglobulin-like receptors (KIRs) immune checkpoint inhibitors such as Lirilumab (BMS-986015), carcinoembryonic antigen cell adhesion molecule (CEACAM) inhibitors (e.g., CEACAM-1, -3 and/or -5). Exemplary anti-CEACAM-1 antibodies are described in WO 2010/125571, WO 2013/082366 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10: 1371/journal.pone.0021146), or cross-reacts with CEACAM-1 and CEACAM-5 as described in, e.g., WO 2013/054331 and US 2014/0271618. Still other checkpoint inhibitors can be molecules directed to B and T lymphocyte attenuator molecule (BTLA), for example as described in Zhang et al., Monoclonal antibodies to B and T lymphocyte attenuator (BTLA) have no effect on in vitro B cell proliferation and act to inhibit in vitro T cell proliferation when presented in a cis, but not trans, format relative to the activating stimulus, Clin Exp Immunol. 2011 January; 163(1): 77-87.

Current chemotherapeutic drugs that may be used in combination with the MUSTANG composition described herein include those used to treat AML including cytarabine (cytosine arabinoside or ara-C) and the anthracycline drugs (such as daunorubicin/daunomycin, idarubicin, and mitoxantrone). Some of the other chemo drugs that may be used to treat AML include: Cladribine (Leustatin®, 2-CdA), Fludarabine (Fludara®), Topotecan, Etoposide (VP-16), 6-thioguanine (6-TG), Hydroxyurea (Hydrea®), Corticosteroid drugs, such as prednisone or dexamethasone (Decadron®), Methotrexate (MTX), 6-mercaptopurine (6-MP), Azacitidine (Vidaza®), Decitabine (Dacogen®). Additional drugs include dasatinib and checkpoint inhibitors such as novolumab, Pembrolizumab, and atezolizumab.

Current chemotherapeutic drugs that may be used in combination with the MUSTANG composition described herein include those used for CLL and other lymphomas including: purine analogs such as fludarabine (Fludara®), pentostatin (Nipent®), and cladribine (2-CdA, Leustatin®), and alkylating agents, which include chlorambucil (Leukeran®) and cyclophosphamide (Cytoxan®) and bendamustine (Treanda®). Other drugs sometimes used for CLL include doxorubicin (Adriamycin®), methotrexate, oxaliplatin, vincristine (Oncovin®), etoposide (VP-16), and cytarabine (ara-C). Other drugs include Rituximab (Rituxan), Obinutuzumab (Gazyva™), Ofatumumab (Arzerra®), Alemtuzumab (Campath®) and Ibrutinib (Imbruvica™).

Current chemotherapeutic drugs that may be used in combination with the MUSTANG composition described herein include those used for CML including: Interferon, imatinib (Gleevec), the chemo drug hydroxyurea (Hydrea®), cytarabine (Ara-C), busulfan, cyclophosphamide (Cytoxan®), and vincristine (Oncovin®). Omacetaxine (Synribo®) is a chemo drug that was approved to treat CML that is resistant to some of the TKIs now in use.

Current chemotherapeutic drugs that may be used in combination with the MUSTANG composition described herein include those used for CMML, for example, Deferasirox (Exjade®), cytarabine with idarubicin, cytarabine with topotecan, and cytarabine with fludarabine, Hydroxyurea (hydroxycarbamate, Hydrea®), azacytidine (Vidaza®) and decitabine (Dacogen®).

Current chemotherapeutic drugs that may be used in combination with the MUSTANG composition described herein include those used for multiple myeloma include Pomalidomide (Pomalyst®), Carfilzomib (Kyprolis™), Everolimus (Afinitor®), dexamethasone (Decadron), prednisone and methylprednisolone (Solu-medrol®) and hydrocortisone.

Current chemotherapeutic drugs that may be used in combination with the MUSTANG composition described herein include those used for Hodgkin's disease include Brentuximab vedotin (Adcetris™): anti-CD-30, Rituximab, Adriamycin® (doxorubicin), Bleomycin, Vinblastine, Dacarbazine (DTIC).

Current chemotherapeutic drugs that may be used in combination with the MUSTANG composition described herein include those used for Non-Hodgkin's disease include Rituximab (Rituxan®), Ibritumomab (Zevalin®), tositumomab (Bexxar®), Alemtuzumab (Campath®) (CD52 antigen), Ofatumumab (Arzerra®), Brentuximab vedotin (Adcetris®) and Lenalidomide (Revlimid®).

Current chemotherapeutic drugs that may be used in combination with the MUSTANG composition described herein include those used for:

B-cell Lymphoma, for example:

Diffuse large B-cell lymphoma: CHOP (cyclophosphamide, doxorubicin, vincristine, and prednisone), plus the monoclonal antibody rituximab (Rituxan). This regimen, known as R-CHOP, is usually given for about 6 months.

Primary mediastinal B-cell lymphoma: R-CHOP.

Follicular lymphoma: rituximab (Rituxan) combined with chemo, using either a single chemo drug (such as bendamustine or fludarabine) or a combination of drugs, such as the CHOP or CVP (cyclophosphamide, vincristine, prednisone regimens. The radioactive monoclonal antibodies, ibritumomab (Zevalin) and tositumomab (Bexxar) are also possible treatment options. For patients who may not be able to tolerate more intensive chemo regimens, rituximab alone, milder chemo drugs (such as chlorambucil or cyclophosphamide).

Chronic lymphocytic leukemia/small lymphocytic lymphoma: R-CHOP.

Mantle cell lymphoma: fludarabine, cladribine, or pentostatin; bortezomib (Velcade) and lenalidomide (Revlimid) and ibrutinib (Imbruvica).

Extranodal marginal zone B-cell lymphoma-mucosa-associated lymphoid tissue (MALT) lymphoma: rituximab; chlorambucil or fludarabine or combinations such as CVP, often along with rituximab.

Nodal marginal zone B-cell lymphoma: rituximab (Rituxan) combined with chemo, using either a single chemo drug (such as bendamustine or fludarabine) or a combination of drugs, such as the CHOP or CVP (cyclophosphamide, vincristine, prednisone regimens. The radioactive monoclonal antibodies, ibritumomab (Zevalin) and tositumomab (Bexxar) are also possible treatment options. For patients who may not be able to tolerate more intensive chemo regimens, rituximab alone, milder chemo drugs (such as chlorambucil or cyclophosphamide).

Splenic marginal zone B-cell lymphoma: rituximab; patients with Hep C-anti-virals.

Burkitt lymphoma: methotrexate; hyper-CVAD-cyclophosphamide, vincristine, doxorubicin (also known as Adriamycin), and dexamethasone. Course B consists of methotrexate and cytarabine; CODOX-M-cyclophosphamide, doxorubicin, high-dose methotrexate/ifosfamide, etoposide, and high-dose cytarabine; etoposide, vincristine, doxorubicin, cyclophosphamide, and prednisone (EPOCH)

Lymphoplasmacytic lymphoma-rituximab.

Hairy cell leukemia-cladribine (2-CdA) or pentostatin; rituximab; interferon-alfa

T-cell lymphomas, for example:

Precursor T-lymphoblastic lymphoma/leukemia-cyclophosphamide, doxorubicin (Adriamycin), vincristine, L-asparaginase, methotrexate, prednisone, and, sometimes, cytarabine (ara-C). Because of the risk of spread to the brain and spinal cord, a chemo drug such as methotrexate is also given into the spinal fluid.

Skin lymphomas: Gemcitabine Liposomal doxorubicin (Doxil); Methotrexate; Chlorambucil; Cyclophosphamide; Pentostatin; Etoposide; Temozolomide; Pralatrexate; R-CHOP.

Angioimmunoblastic T-cell lymphoma: prednisone or dexamethasone.

Extranodal natural killer/T-cell lymphoma, nasal type: CHOP.

Anaplastic large cell lymphoma: CHOP; pralatrexate (Folotyn), targeted drugs such as bortezomib (Velcade) or romidepsin (Istodax), or immunotherapy drugs such as alemtuzumab (Campath) and denileukin diftitox (Ontak).

Primary central nervous system (CNS) lymphoma-methotrexate; rituximab.

A more general list of suitable chemotherapeutic agents includes, but are not limited to, radioactive molecules, toxins, also referred to as cytotoxins or cytotoxic agents, which includes any agent that is detrimental to the viability of cells, agents, and liposomes or other vesicles containing chemotherapeutic compounds. Examples of suitable chemotherapeutic agents include but are not limited to 1-dehydrotestosterone, 5-fluorouracil decarbazine, 6-mercaptopurine, 6-thioguanine, actinomycin D, adriamycin, aldesleukin, alkylating agents, allopurinol sodium, altretamine, amifostine, anastrozole, anthramycin (AMC)), anti-mitotic agents, cis-dichlorodiamine platinum (II) (DDP) cisplatin), diamino dichloro platinum, anthracyclines, antibiotics, antis, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunorubicin HCl, daunorucbicin citrate, denileukin diftitox, Dexrazoxane, Dibromomannitol, dihydroxy anthracin dione, Docetaxel, dolasetron mesylate, doxorubicin HCl, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCl, glucocorticoids, goserelin acetate, gramicidin D, granisetron HCl, hydroxyurea, idarubicin HCl, ifosfamide, interferon α-2b, irinotecan HCl, letrozole, leucovorin calcium, leuprolide acetate, levamisole HCl, lidocaine, lomustine, maytansinoid, mechlorethamine HCl, medroxyprogesterone acetate, megestrol acetate, melphalan HCl, mercaptipurine, mesna, methotrexate, methyltestosterone, mithramycin, mitomycin C, mitotane, mitoxantrone, nilutamide, octreotide acetate, ondansetron HCl, paclitaxel, pamidronate disodium, pentostatin, pilocarpine HCl, plimycin, polifeprosan 20 with carmustine implant, porfimer sodium, procaine, procarbazine HCl, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Additional therapeutic agents that can be administered in combination with the MUSTANG compositions disclosed herein can include bevacizumab, sutinib, sorafenib, 2-methoxyestradiol, finasunate, vatalanib, vandetanib, aflibercept, volociximab, etaracizumab, cilengitide, erlotinib, cetuximab, panitumumab, gefitinib, trastuzumab, atacicept, rituximab, alemtuzumab, aldesleukine, atlizumab, tocilizumab, temsirolimus, everolimus, lucatumumab, dacetuzumab, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, oblimersen, plitidepsin, talmapimod, enzastaurin, tipifarnib, perifosine, imatinib, dasatinib, lenalidomide, thalidomide, simvastatin, and celecoxib.

In one aspect of the present invention, the MUSTANG compositions disclosed herein are administered in combination with at least one immunosuppressive agent. The immunosuppressive agent may be selected from the group consisting of a calcineurin inhibitor, e.g. a cyclosporin or an ascomycin, e.g. Cyclosporin A (NEORAL®), tacrolimus, a mTOR inhibitor, e.g. rapamycin or a derivative thereof, e.g. Sirolimus (RAPAMUNE®), Everolimus (Certican®), temsirolimus, biolimus-7, biolimus-9, a rapalog, e.g. azathioprine, campath 1H, a S1P receptor modulator, e.g. fingolimod or an analogue thereof, an anti-IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, 15-deoxyspergualin, tresperimus, Leflunomide ARAVA®, anti-CD25, anti-IL2R, Basiliximab (SIMULECT®), Daclizumab (ZENAPAX®), mizorbine, methotrexate, dexamethasone, pimecrolimus (Elidel®), abatacept, belatacept, etanercept (Enbrel®), adalimumab (Humira®), infliximab (Remicade®), an anti-LFA-1 antibody, natalizumab (Antegren®), Enlimomab, ABX-CBL, antithymocyte immunoglobulin, siplizumab, and efalizumab.

In one aspect of the present invention, the MUSTANG composition described herein can be administered in combination with at least one anti-inflammatory agent. The anti-inflammatory agent can be a steroidal anti-inflammatory agent, a nonsteroidal anti-inflammatory agent, or a combination thereof. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide di sodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

In one aspect of the present invention, the MUSTANG composition described herein can be administered in combination with at least one immunomodulatory agent.

### Methods of Manufacturing MUSTANG Compositions

T-cell subpopulations specific for a single TAA to be combined into the MUSTANG compositions for therapeutic administration described herein can be generated using any known method in the art or as described herein. Activated T-cell subpopulations that recognize at least one epitope of an antigen of a tumor can be generated by any method known in the art or as described herein. Non-limiting exemplary methods of generating activated T-cell subpopulations that recognize at least one epitope of an antigen of a tumor can be found in, for example Shafer et al., Leuk Lymphoma (2010) 51(5):870-880; Cruz et al., Clin Cancer Res., (2011) 17(22): 7058-7066; Quintarelli et al., Blood (2011) 117(12): 3353-3362; and Chapuis et al., Sci Transl Med (2013) 5(174):174ra27, all incorporated herein by reference.

Generally, generating the T-cell subpopulations of the MUSTANG compositions of the present invention may involve (i) collecting a peripheral blood mononuclear cell product from a donor; (ii) determining the HLA subtype of the mononuclear cell product; (iii) separating the monocytes and the lymphocytes of the mononuclear cell product; (iv) generating and maturing dendritic cells (DCs) from the monocytes; (v) pulsing the DCs with a TAA; (vi) optionally carrying out a CD45RA+ selection to isolate naïve lymphocytes; (vii) stimulating the naïve lymphocytes with the peptide-pulsed DCs in the presence of a cytokine cocktail; (viii) repeating the T cell stimulation with fresh peptide-pulsed DCs or other peptide-pulsed antigen presenting cells in the presence of a cytokine cocktail; (ix) harvesting the TAA-L and cryopreserving for future use.

In some aspects, generating the T-cell subpopulations of the MUSTANG compositions of the present invention may involve (i) collecting a peripheral blood mononuclear cell product from a donor; (ii) determining the HLA subtype of the mononuclear cell product; (iii) separating the monocytes and the lymphocytes of the mononuclear cell product; (iv) generating and maturing dendritic cells (DCs) from the monocytes; (v) pulsing the DCs with a TAA; (vi) optionally carrying out a CD45RA+ selection to isolate naïve T cells; (vii) stimulating the naïve T cells with the peptide-pulsed DCs in the presence of a cytokine cocktail; (viii) repeating the T cell stimulation with fresh peptide-pulsed DCs or other peptide-pulsed antigen presenting cells in the presence of a cytokine cocktail; (ix) harvesting the TAA-L and cryopreserving for future use.

Collecting a Peripheral Blood Mononuclear Cell Product from a Donor

The generation of T-cell subpopulations to be specific to a single TAA generally requires a peripheral blood mononuclear cell (PBMC) product from a donor, either an allogeneic or autologous donor, as a starting material. Isolation of PBMCs is well known in the art. Non-limiting exemplary methods of isolating PBMCs are provided in Grievink, H. W., et al. (2016) “Comparison of three isolation techniques for human peripheral blood mononuclear cells: Cell recovery and viability, population composition, and cell functionality,” Biopreservation and BioBanking, which is incorporated herein by reference. The PBMC product can be isolated from whole blood, an apheresis sample, a leukapheresis sample, or a bone marrow sample provided by a donor. In some embodiments, the starting material is an apheresis sample, which provides a large number of initially starting mononuclear cells, potentially allowing a large number of different T-cell subpopulations to be generated. In some embodiments, the PBMC product is isolated from a sample containing peripheral blood mononuclear cells (PBMCs) provided by a donor. In some embodiments, the donor is a healthy donor. In some embodiments, the PBMC product is derived from cord blood. In some embodiments, the donor is the same donor providing stem cells for a hematopoietic stem cell transplant (HSCT).

### Determining HLA Subtype

When the T-cell subpopulations are generated from an allogeneic, healthy donor, the HLA subtype profile of the donor source is determined and characterized. Determining HLA subtype (i.e., typing the HLA loci) can be performed by any method known in the art. Non-limiting exemplary methods for determining HLA subtype can be found in Lange, V., et al., BMC Genomics (2014)15: 63; Erlich, H., Tissue Antigens (2012) 80:1-11; Bontadini, A., Methods (2012) 56:471-476; Dunn, P. P., Int J Immunogenet (2011) 38:463-473; and Hurley, C. K., “DNA-based typing of HLA for transplantation.” in Leffell, M. S., et al., eds., Handbook of Human Immunology, 1997. Boca Raton: CRC Press, each independently incorporated herein by reference. Preferably, the HLA-subtyping of each donor source is as complete as possible.

In some embodiments, the determined HLA subtypes include at least 4 HLA loci, preferably HLA-A, HLA-B, HLA-C, and HLA-DRB1. In some embodiments, the determined HLA subtypes include at least 6 HLA loci. In some embodiments, the determined HLA subtypes include at least 6 HLA loci. In some embodiments, the determined HLA subtypes include all of the known HLA loci. In general, typing more HLA loci is preferable for practicing the invention, since the more HLA loci that are typed, the more likely the allogeneic T-cell subpopulations selected will have highest activity relative to other allogeneic T-cell subpopulations that have HLA alleles or HLA allele combinations in common with the patient or the diseased cells in the patient.

### Separating the Monocytes and the Lymphocytes of the Peripheral Blood Mononuclear Cell Product

In general, the PBMC product may be separated into various cell-types, for example, into platelets, red blood cells, lymphocytes, and monocytes, and the lymphocytes and monocytes retained for initial generation of the T-cell subpopulations. The separation of PBMCs is known in the art. Non-limiting exemplary methods of separating monocytes and lymphocytes include Vissers et al., J Immunol Methods. 1988 Jun. 13; 110(2):203-7 and Wahl et al., Current Protocols in Immunology (2005) 7.6A.1-7.6A.10, which are incorporated herein by reference. For example, the separation of the monocytes can occur by plate adherence, by CD14+ selection, or other known methods. The monocyte fraction is generally retained in order to generate dendritic cells used as an antigen presenting cell in the T-cell subpopulation manufacture. The lymphocyte fraction of the PBMC product can be cryopreserved until needed, for example, aliquots of the lymphocyte fraction (˜5×10⁷ cells) can be cryopreserved separately for both Phytohemagglutinin (PHA) Blast expansion and T-cell subpopulation generation.

### Generating Dendritic Cells

The generation of mature dendritic cells used for antigen presentation to prime T-cells is well known in the art. Non-limiting exemplary methods are included in Nair et al., “Isolation and generation of human dendritic cells.” Current protocols in immunology (2012) 0 7: Unit7.32. doi:10.1002/0471142735.im0732s99 and Castiello et al., Cancer Immunol Immunother, 2011 April; 60(4):457-66, which are incorporated herein by reference. For example, the monocyte fraction can be plated into a closed system bioreactor such as the Quantum Cell Expansion System, and the cells allowed to adhere for 2-4 hours at which point 1,000 U/mL of IL-4 and 800 U/mL GM-CSF can be added. The concentration of GM-CSF and IL-4 can be maintained. The dendritic cells can be matured using a cytokine cocktail. In some embodiments the cytokine cocktail consists of LPS (30 ng/mL), IL-4 (1,000 U/mL), GM-CSF (800 U/mL), TNF-Alpha (10 ng/mL), IL-6 (100 ng/mL), and IL-1beta (10 ng/mL). The dendritic cell maturation generally occurs in 2 to 5 days. In some embodiments, the adherent DCs are harvested and counted using a hemocytometer. In some embodiments, a portion of the DCs are cryopreserved for additional further stimulations.

### Pulsing the Dendritic Cells

The non-mature and mature dendritic cells are pulsed with one or more peptides, of a single TAA.

In some embodiments, the TAA peptides used to pulse the non-mature and mature dendritic cells are from a library of overlapping peptide fragments of the tumor antigen, as provided for example, in commercially available overlapping peptide libraries. In some embodiments, the TAA peptides used to the TAA peptides used to pulse the non-mature and mature dendritic cells are from a library of overlapping peptide fragments of the tumor antigen, as provided for example, in commercially available overlapping peptide libraries, wherein the library been further enriched with one or more specific known or identified epitopes expressed by the patient's tumor. In some embodiments, the TAA peptides used to pulse the non-mature and mature dendritic cells are from specifically selected HLA-restricted peptides generated by determining the HLA profile of the donor source, and including peptide epitopes derived from the targeted TAA that are active through the donor's HLA type. Methods of pulsing a dendritic cell with a TAA are known. For example, about 100 ng of one or more peptides of the TAA, for example a peptide library, can be added per 10 million dendritic cells and incubated for about 30 to 120 minutes.

### Naïve T-Cell Selection of Lymphocytes

In order to increase the potential number of specific TAA activated T-cells and reduce T-cells that target other antigens, it is preferable to utilize naïve T-cells as a starting material. To isolate naïve T-cells, the lymphocytes can undergo a selection, for example CD45RA+ cells selection. CD45RA+ cell selection methods are generally known in the art. Non-limiting exemplary methods are found in Richards et al., Immune memory in CD4+CD45RA+ T cells. Immunology. 1997; 91(3):331-339 and McBreen et al., J Virol. 2001 May; 75(9): 4091-4102, which are incorporated herein by reference. For example, to select for CD45RA⁺ cells, the cells can be labeled using 1 vial of CD45RA microbeads from Miltenyi Biotec per 1×10¹¹ cells after 5-30 minutes of incubation with 100 mL of CliniMACS buffer and approximately 3 mL of 10% human IVIG, 10 ug/mL DNAase I, and 200 mg/mL of magnesium chloride. After 30 minutes, cells will be washed sufficiently and resuspended in 20 mL of CliniMACS buffer. The bag will then be set up on the CLINIMACS Plus device and the selection program can be run according to manufacturer's recommendations. After the program is completed, cells can be counted, washed and resuspended in “CTL Media” consisting of 44.5% EHAA Click's, 44.5% Advanced RPMI, 10% Human Serum, and 1% GlutaMAX.

Stimulating Naïve T Cells with Peptide-Pulsed Dendritic Cells

Prior to stimulating naïve T-cells with the dendritic cells, it may be preferable to irradiate the DCs, for example, at 25 Gy. The DCs and naïve T-cells are then co-cultured. The naïve T-cells can be co-cultured in a ratio range of DCs to T cells of about 1:5-1:50, for example, 1:5; 1:10, 1:15, 1:20, 1:25, 1:30, 1:35, 1:40, 1:45, or about 1:50. The DCs and T-cells are generally co-cultured with cytokines. In some embodiments, the cytokines are selected from a group consisting of IL-6 (100 ng/mL), IL-7 (10 ng/mL), IL-15 (5 ng/mL), IL-12 (10 ng/mL), and IL-21 (10 ng/mL).

Second T Cell Stimulation

In general, it is preferable to further stimulate the T-cell subpopulations with one or additional stimulation procedures. The additional stimulation can be performed with, for example, fresh DCs pulsed with the same peptides as used in the first stimulation, similarly to as described above. In some embodiments, the cytokines used during the second stimulation are selected from a group consisting of IL-7 (10 ng/mL) and IL-2 (100 U/mL).

Alternatively, peptide-pulsed PHA blasts can be used as the antigen presenting cell. The use of peptide-pulsed PHA blasts to stimulate and expand T-cells are well known in the art Non-limiting exemplary methods can be found in Weber et al., Clin Cancer Res. 2013 Sep. 15; 19(18): 5079-5091 and Ngo et al., J Immunother. 2014 May; 37(4): 193-203, which are incorporated herein by reference. The peptide-pulsed PHA blasts can be used to expand the T-cell subpopulation in a ratio range of PHA blasts to expanded T cells of 10:1-1:10. For example, the ratio of PHA blasts to T cells can be 10:1, between 10:1 and 9:1, between 9:1 and 8:1, between 8:1 and 7:1, between 7:1 and 6:1, between 6:1 and 5:1, between 5:1 and 4:1, between 4:1 and 3:1, between 3:1 and 2:1, between 2:1 and 1:1, between 1:1 and 1:2, between 1:2 and 1:3, between 1:3 and 1:4, between 1:4 and 1:5, between 1:5 and 1:6, between 1:6 and 1:7, between 1:7 and 1:8, between 1:8 and 1:9, between 1:9 and 1:10. In general, cytokines are included in the co-culture, and are selected from the group consisting of IL-7 (10 ng/mL) and IL-2 (100 U/mL).

### Additional T-Cell Expansion and T-Cell Subpopulation Harvest

Additional T cell stimulations may be necessary to generate the necessary number of T-cell subpopulations for use in the MUSTANG composition. Following any stimulation and expansion, the T-cell subpopulations are harvested, washed, and concentrated. In some embodiments, a solution containing a final concentration of 10% dimethyl sulfoxide (DMSO), 50% human serum albumin (HSA), and 40% Hank's Balanced Salt Solution (HBSS) will then be added to the cryopreservation bag. In some embodiments, the T-cell subpopulation will be cryopreserved in liquid nitrogen.

### Further Characterization of the T-Cell Subpopulation

The T-cell subpopulations for use in the MUSTANG composition of the present invention are HLA-typed and can be further characterized prior to use or inclusion in the MUSTANG composition. For example, each of the T-cell subpopulations may be further characterized by, for example, one or more of i) determining the TAA specificity of the T-cell subpopulation; ii) identifying the tumor associated antigen epitope(s) the T-cell subpopulation is specific to; iii) determining whether the T-cell subpopulation includes MHC Class I or Class II restricted subsets or a combination of both; iv) correlating antigenic activity through the T-cell's corresponding HLA-allele; and v) characterizing the T-cell subpopulation's immune effector subtype concentration, for example, the population of effector memory cells, central memory cells, γδ T-cells, CD8+, CD4+, NKT-cell.

### Determining the Tumor Associated Antigen Specificity of the T-Cell Subpopulation

The T-cell subpopulations of the MUSTANG composition can be further characterized by determining each T-cell subpopulation's specificity for its targeted tumor antigen. Specificity can be determined using any known procedure, for example, an ELISA based immunospot assay (ELISpot). In some embodiments, tumor-associated antigen specificity of the T-cell subpopulation is determined by ELISpot assay. ELISpot assays are widely used to monitor adaptive immune responses in both humans and animals. The method was originally developed from the standard ELISA assay to measure antibody secretion from B cells (Czerkinsky C. et al. (1983) A solid-phase enzyme-linked immunospot (ELISPOT) assay for enumeration of specific antibody-secreting cells. J. Immunol Methods 65: 109-21), which is incorporated herein by reference. The assay has since been adapted to detect secreted cytokines from T cells, for example IFN-γ, and is an essential tool for understanding the helper T cell response.

A T-cell ELISpot assay generally comprises the following steps:

i) a capture antibody specific for the chosen analyte, for example IFN-γ, is coated onto a PVDF plate;

ii) the plate is blocked, usually with a serum;

iii) the T-cell subpopulation is added along with the specific, targeted tumor associated antigen;

iv) plates are incubated and secreted cytokines, for example IFN-γ, are captured by the immobilized antibody on the PVDF surface;

v) after washing, a biotinylated detection antibody is added to allow detection of the captured cytokine; and

vi) the secreted cytokine is visualized using an avidin-HRP or avidin-ALP conjugate and a colored precipitating substrate.

Each colored spot represents a cytokine secreting cell. The spots can be counted by eye or by using an automated plate-reader. Many different cytokines can be detected using this method including IL-2, IL-4, IL-17, IFN γ, TNFα, and granzyme B. The size of the spot is an indication of the per cell productivity and the avidity of the binding. The higher the avidity of the T cell recognition the higher the productivity resulting in large, well-defined spots.

### Identifying the TAA Epitope(s) the T-Cell Subpopulation is Specific to

The T-cell subpopulations of the MUSTANG composition can be further characterized by identifying the specific TAA epitope or epitopes to which the T-cell subpopulation is specific to. This may be especially useful when more than one TAA peptide is used to prime the T-cell subpopulation. Determining TAA epitope specificity is generally known in the art. Non-limiting exemplary methods include Ohminami et al., Blood. 2000 Jan. 1; 95(1):286-93; Oka et al., Immunogenetics. 2000 February; 51(2):99-107; and Bachinsky et al., Cancer Immun. 2005 Mar. 22; 5:6, which are each incorporated herein by reference. For example, to identify the epitopes with TAA specific activity antigen peptide libraries can be grouped into pools in which each peptide is represented in two or more pools as a quick screening tool in an Elispot assay, and the pools showing activity determined. Common peptides represented in both pools can then be further screened to identify the specific peptide epitopes which show activity.

### Determining the T-Cell Subpopulation's MHC-Class I or Class II Restricted Subsets

The T-cell subpopulations of the MUSTANG composition can be further characterized by determining the subpopulation's MHC Class I or Class II subset restriction response. This is done to determine whether epitope recognition is mediated by CD8+(class I) or CD4+(class II) T-cells. General methods for determining the MHC Class I or Class II response are generally known in the art. A non-limiting exemplary method is found in Weber et al., Clin Cancer Res. 2013 Sep. 15; 19(18): 5079-5091, which is incorporated herein by reference. For example, to determine HLA restriction response, T cells can be pre-incubated with class I or II blocking antibodies for 1 hour before the addition of antigen peptides in an ELISPOT assay using autologous peptide-pulsed PHA blasts as targets with unpulsed PHA blasts as a control. IFNγ-secretion is measured in the presence of each blocking antibody. If, when pre-incubated with a class I blocking antibody, IFNγ-secretion is reduced to background levels then this is indicative of a class I restriction and the epitope recognition is mediated by CD8+ T cells. If, when pre-incubated with a class II blocking antibody, IFNγ-secretion is reduced to background levels then this is indicative of a class II restriction and the epitope recognition is mediated by CD4+ T cells.

The direct detection of antigen-specific T cells using tetramers of soluble peptide-major histocompatibilty complex (pMHC) molecules is widely used in both basic and clinical immunology. Tetrameric complexes of HLA molecules can be used to stain antigen-specific T cells in FACS analysis. In vitro synthesized soluble HLA-peptide complexes are used as tetrameric complexes to stain antigen specific T cells in FACS analysis (Altman et al., Science 274: 94-96, 1996). T-cell subpopulations specific for TAAs are stained with CD8 fluorescein isothiocyanate (FITC) and with phycoerythrin (PE)-labeled MHC pentamers at various timepoints during in vitro stimulation. Antigen specificity is measured by flow cytometry.

### Correlating Antigenic Activity Through the T-Cell's Corresponding HLA-Allele

The T-cell subpopulation can be further characterized by correlating antigenic activity through the T-cell subpopulation's corresponding HLA-allele. Correlating antigenic activity through the corresponding HLA-allele can be done using any known method. For example, In some embodiments, a HLA restriction assay is used to determine antigen activity through a corresponding allele. Methods to determine T cell restriction are known in the art and involve inhibition with locus specific antibodies, followed by antigen presentation assays (ELISPOT) with panels of cell lines matched or mismatched at the various loci of interest (see, e.g., (Oseroff et al., J Immunol (2010) 185(2): 943-955; Oseroff et al., J Immunol (2012) 189(2): 679-688; Wang Curr Protocols in immunol (2009) Chap. 20, page 10; Wilson et al., J. Virol. (2001) 75(9): 4195-4207), each independently incorporated herein by reference. Because epitope binding to HLA class II molecules is absolutely necessary (but not sufficient) for T cell activation, data from in vitro HLA binding assays has also been useful to narrow down the possible restrictions (Arlehamn et al., J Immunol (2012b) 188(10):5020-5031). This is usually accomplished by testing a given epitope for binding to the specific HLA molecules expressed in a specific donor and eliminating from further consideration HLA molecules to which the epitope does not bind. To determine the HLA restriction of the identified epitope, T cells can be plated in an IFN-γ ELISPOT assay with TAA peptide pulsed PHA blasts that match at a single allele, measuring the strongest antigen activity, and identifying the corresponding allele.

### Characterizing the T-Cell Subpopulation's Immune Effector Subtype Concentration

The T-cell subpopulation is likely to be made up of different lymphocytic cell subsets, for example, a combination of CD4+ T-cells, CD8+ T-cells, CD3+/CD56+ Natural Killer T-cells (CD3+ NKT), and TCR γδ T-cells (γδ T-cells). In particular, the T-cell subpopulation likely include at least CD4+ T-cells and CD8+ T-cells that have been primed and are capable of targeting a single specific TAA for tumor killing and/or cross presentation. The T-cell subpopulation may further comprise activated γδ T-cells and/or activated CD3+/CD56+ NKT cells capable of mediating anti-tumor responses. Accordingly, the T-cell subpopulation may be further characterized by determining the population of various lymphocytic subtypes, and the further classification of such subtypes, for example, by determining the presence or absence of certain clusters of differentiation (CD) markers, or other cell surface markers, expressed by the cells and determinative of cell subtype.

In some embodiments, the T-cell subpopulation may be analyzed to determine CD8+ T-cell population, CD4+, T-cell population, γδ T-cell population, NKT-cell population, and other populations of lymphocytic subtypes. For example, the population of CD4+ T-cells within the T-cell subpopulation may be determined, and the CD4+ T-cell subtypes further determined. For example, the CD4+ T-cell population may be determined, and then further defined, for example, by identifying the population of T-helper 1 (Th₁), T-helper 2 (Th₂), T-helper 17 (Th₁₇), regulatory T cell (T_{reg}), follicular helper T-cell (T_{fh}), and T-helper 9 (Th₉). Likewise, the other lymphocytic subtypes comprising the T-cell subpopulation can be determined and further characterized.

In addition, the T-cell subpopulation can be further characterized, for example, for the presence, or lack thereof, of one or more markers associated with, for example, maturation or exhaustion. T cell exhaustion (Tₑₓ) is a state of dysfunction that results from persistent antigen and inflammation, both of which commonly occur in tumor tissue. The reversal or prevention of exhaustion is a major area of research for tumor immunotherapy. Tₑₓ cell populations can be analyzed using multiple phenotypic parameters, either alone or in combination. Hallmarks commonly used to monitor T cell exhaustion are known in the art and include, but are not limited to, programmed cell death-1 (PD-1), CTLA-4/CD152 (Cytotoxic T-Lymphocyte Antigen 4), LAG-3 (Lymphocyte activation gene-3; CD223), TIM-3 (T cell immunoglobulin and mucin domain-3), 2B4/CD244/SLAMF4, CD160, and TIGIT (T cell Immunoreceptor with Ig and ITIM domains).

The T-cell subpopulations of the described compositions described herein can be subjected to further selection, if desired. For example, a particular T-cell subpopulation for inclusion in a MUSTANG composition described herein can undergo further selection through depletion or enriching for a sub-population. For example, following priming, expansion, and selection, the cells can be further selected for other cluster of differentiation (CD) markers, either positively or negatively. For example, following selection of for example CD4+ T-cells, the CD4+ T-cells can be further subjected to selection for, for example, a central memory T-cells (T_{cm}). For example, the enrichment for CD4+T_{cm} cells comprises negative selection for cells expression a surface marker present on naïve T cells, such as CD45RA, or positive selection for cells expressing a surface marker present on T_{cm} cells and not present on naïve T-cells, for example CD45RO, CD62L, CCR7, CD27, CD127, and/or CD44. In addition, the T-cell subpopulations described herein can be further selected to eliminate cells expressing certain exhaustion markers, for example, programmed cell death-1 (PD-1), CTLA-4/CD152 (Cytotoxic T-Lymphocyte Antigen 4), LAG-3 (Lymphocyte activation gene-3; CD223), TIM-3 (T cell immunoglobulin and mucin domain-3), 2B4/CD244/SLAMF4, CD160, and TIGIT (T cell Immunoreceptor with Ig and ITIM domains)

Methods for characterizing lymphocytic cell subtypes are well known in the art, for example flow cytometry, which is described in Pockley et al., Curr Protoc Toxicol. 2015 Nov. 2; 66:18.8.1-34, which is incorporated herein by reference.

### Identifying the MUSTANG Composition Most Suitable for Administration

Characterization of each T-cell subpopulation composition allows for the selection of the most appropriate T-cell subpopulations for inclusion in the MUSTANG composition for any given patient. The goal is to match the product with the patient that has the both the highest HLA match and greatest TAA activity through the greatest number of shared alleles. In some embodiments, the T-cell subpopulation has at least one shared allele or allele combination with TAA activity through that allele or allele combination. In some embodiments, the T-cell subpopulation has greater than 1 shared allele or allele combination with TAA activity through that allele or allele combination. In some embodiments, the T-cell subpopulation with the most shared alleles or allele combinations and highest specificity through those shared alleles and allele combinations is provided to a human in need thereof. For example, if T-cell subpopulation 1 is a ⅝ HLA match with the patient with TAA activity through 3 shared alleles or allele combinations while T-cell subpopulation 2 is a 6/8 HLA match with the patient with TAA activity through 1 shared allele the skilled practitioner would select T-cell subpopulation 1 as it has TAA activity through a greater number of shared alleles.

### Testing T-Cell Subpopulations or MUSTANG Composition Reactivity Against Patient's Tumor

The cytolytic activity of an activated T-cell subpopulation or the MUSTANG composition against a patient's tumor can be evaluated. A method of testing reactivity of T-cell subpopulations against tumor cells are well known. Non-limiting exemplary methods include Jedema et al., Blood (2004) 103:2677-2682; Noto et al., J Vis Exp. 2013; (82): 51105 and Baumgaertner et al., Bio-protocol “Chromium-51 (⁵¹Cr) Release Assay to Assess Human T Cells for Functional Avidity and Tumor Cell Recognition.” (2016) 6(16): e1906. For example, the T-cell subpopulation can be incubated with the patient's tumor and the percent lysis of the tumor cells determined. For example, a biopsy or blood sample will be collected from the patient. Target cells from the patient are fluorescence labeled with carboxyfluorescein succinimidyl ester (CFSE, Invitrogen), peptide-pulsed and incubated with activated T-cell subpopulations or MUSTANG composition at a 40:1 effector-to-target ratios for 6-8 hrs. Ethidium homodimer (Invitrogen) is added after incubation to stain dead cells. Samples are acquired on a BD Fortessa Flow Cytometer. The number of live target cells is determined by gating on carboxyfluorescein succinimidyl ester-positive, ethidium homodimer-negative cells, and used to calculate cytolytic activity as follows: Lysis (%)=100−((live target cells/sample/live target cells control)×100).

T-cell subpopulations or MUSTANG compositions with the highest levels of reactivity against a patient's tumor can be selected for administration to the patient, providing a higher likelihood of successful therapeutic efficacy.

### Banked T-Cell Subpopulations Directed to Single Tumor Associated Antigens

The establishment of a T-cell subpopulation bank comprising discrete, characterized T-cell subpopulations for selection and inclusion in a MUSTANG composition bypasses the need for an immediately available donor and eliminates the wait required for autologous T cell production. Preparing T-cell subpopulations directed to specific, known tumor antigens by using donors, for example healthy volunteers or cord blood, allows the production and banking of T-cell subpopulations readily available for administration. Because the T-cell subpopulations are characterized, the selection of suitable T-cell subpopulations can be quickly determined based on minimal information from the patient, for example HLA-subtype and, optionally TAA expression profile.

From a single donor a T cell composition can be generated for use in multiple patients who share HLA alleles that have activity towards a specific TAA. The T-cell subpopulation bank of the present invention includes a population of T-cell subpopulations which have been characterized as described herein. For example, the T-cell subpopulations of the bank are characterized as to HLA-subtype and one or more of i) TAA specificity of the T-cell subpopulation; ii) TAA epitope(s) the T-cell subpopulation is specific to; iii) T-cell subpopulation MHC Class I and Class II restricted subsets; iv) antigenic activity through the T-cell's corresponding HLA-allele; and v) immune effector subtype concentration, for example, the population of effector memory cells, central memory cells, γδ T-cells, CD8+, CD4+, NKT-cell.

In some embodiments, the present invention is a method of generating a T-cell subpopulation bank comprising: (i) obtaining eligible donor samples; (ii) generating T-cell subpopulations specific to a single TAA; (iii) characterizing the T-cell subpopulation; (iv) cryopreserving the T-cell subpopulation; and (v) generating a database of T-cell subpopulation composition characterization data. In some embodiments, the T-cell subpopulations are stored according to their donor source. In some embodiments, the T-cell subpopulations are stored by TAA specificity. In some embodiments, the T-cell subpopulations are stored by human leukocyte antigen (HLA) subtype and restrictions.

The banked T-cell subpopulations described herein are used to comprise a MUSTANG composition for administration to a tumor patient following the determination of the patient's HLA subtype and, optionally, TAA expression profile of the patient's tumor.

### EXAMPLES

### Example 1. Generation of T-Cell Subpopulations from Peripheral Blood Using Multiple-TAA Overlapping Peptides

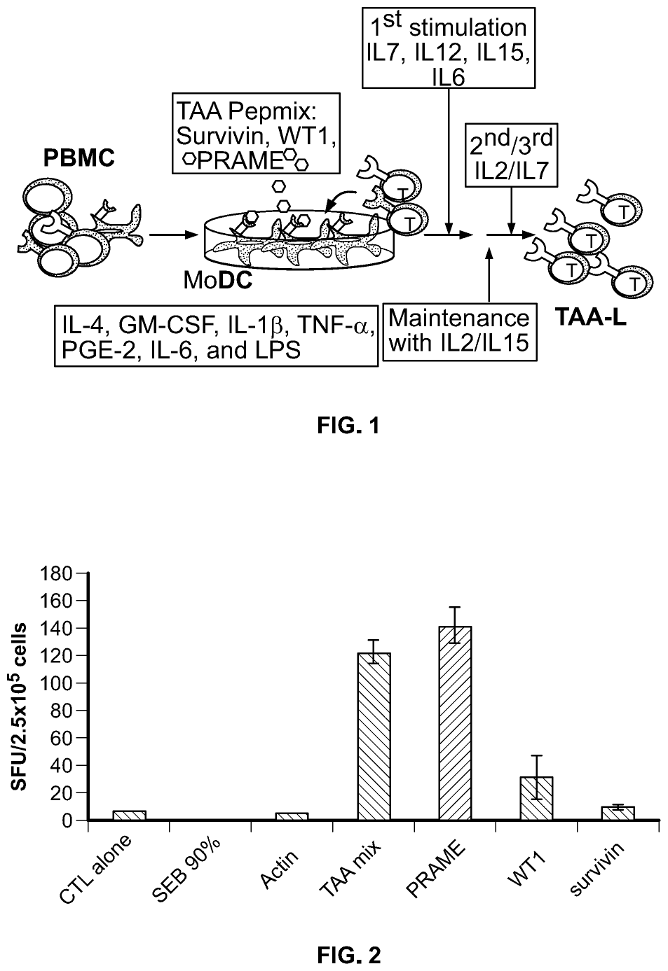

TAA-specific T-cell lines were generated from total human blood peripheral mononuclear cells (Step 1). Matured dendritic cells (DCs) were harvested and used as antigen presenting cells (APCs) and peptide-pulsed with a mix of three overlapping peptide libraries for WT1, Survivin, and PRAME (Step 2). T-cells were initially stimulated using a cytokine mix containing IL-7, IL-12, IL-15, IL-6, and IL-27 (Step 3). Subsequent stimulations (Steps 4 and 5) were performed using irradiated DCs or irradiated phytohemagglutinin (PHA) blasts. See generally FIG. 1. Experimental procedures for each of these steps are provided below.

Step 1. Isolation of Mononuclear Cells

Heparinized peripheral blood was diluted in an equal volume of warm RPMI 1641 (Invitrogen) or PBS. In a 50 mL centrifuge tube, 10-15 mL of Lymphoprep (Axis-Shield) was overlayed with 20-30 mL of diluted blood. The mixture was centrifuged at 800×g for 20 minutes or 400×g for 40 minutes at ambient temperature, ensuring that acceleration and deceleration were set to “1” to prevent disrupting the interface. 1 mL of plasma aliquots were saved and stored at −80° C. The peripheral blood mononuclear cell (PBMC) interface was harvested into an equal volume of RPMI 1640, centrifuged at 450×g for 10 minutes at ambient temperature, and the supernatant was aspirated. The pellet was loosened and the cells were resuspended in a volume of RPMI 1640 or PBS that yields and estimated 10×10⁶ cells/mL. An aliquot of cells was removed for counting using 50% red cell lysis buffer or Trypan blue and using a hemocytometer. The PBMCs were saved for DC generation using adherence (Step 2 below) and non-adherent cells were cryopreserved for use at initiation.

Step 2. Dendritic Cell (DC) Generation

PBMCs were centrifuged at 400×g for 5 minutes at ambient temperature, and the supernatant was aspirated. The cells were resuspended at approximately 5×10⁶ cells/mL in CellGenix DC medium containing 2 mM of Glutamax (Invitrogen), and the cells were plated in a 6-well plate (2 mL/well). The PBMC non-adherent fraction was removed after 1-2 hours, and the wells were rinsed with 2-5 mL of CellGenix DC medium or PBS and added to the harvested medium/non-adherent fraction. The non-adherent fraction was saved for later cryopreservation. 2 mL of DC medium containing 1,000 U/mL of IL-4 (R&D Systems) and 800 U/mL GM-CSF (CNMC Pharmacy) was added back to the adherent cells. All surrounding wells were filled with approximately 2 mL of sterile water or PBS to maintain the humidity within the plate, and the plate was placed in the incubator at 37° C. and 5% CO₂. On day 3 to 4, the cells are fed with 1,000 U/mL IL-4 and 800 U/mL GM-CSF. On day 5 to 6, the DCs were matured in 2 mL/well of DC medium containing lipopolysaccharide (LPS, Sigma) (30 ng/mL), IL-4 (1,000 U/mL), GM-CSF (800 U/mL), TNF-α (10 ng/mL, R&D Systems), IL-6 (100 ng/mL, CellGenix), and IL-1β (10 ng/mL, R&D Systems). The mature DCs were harvested on day 7 to 8 by gentle resuspension. The cells were counted using a hemocytometer. The DCs were transferred to a 15 mL centrifuge tube and centrifuged for 5 minutes at 400×g at ambient temperature. The supernatant was aspirated, and the pellet was resuspended by finger flicking, and 100 μL of appropriate overlapping peptide library Mastermix (200 ng/peptide in 200 μL; PRAME, WT1, and Survivin PepMix™; JPT Peptide Technologies) per 1-5×10⁶ cells was added to the CDs. The DCs and overlapping peptide libraries were mixed and transferred to the incubator. The mixture was incubated for 60-90 minutes at 37° C. and 5% CO₂.

Step 3. T-Cell Population Initiation

After pulsing with the overlapping peptide libraries, DCs were irradiated at 25 Gy. The DCs were washed with DC medium and centrifuged at 400×g for 5 minutes at ambient temperature. The supernatant was aspirated, and the wash step was repeated twice more. The cells were counted using a hemocytometer. The DCs were resuspended at 2-4×10⁵ cells/mL of CTL medium with 10% human serum (HS, Valley) for initiation. 1 mL of irradiated DCs/well were plated in a 24-well tissue culture treated plate.

Previously-frozen PBMCs from Step 1 were thawed at 37° C. and diluted in 10 mL of warm medium/1 mL of frozen cells. The PBMCs were centrifuged at 400×g for 5 minutes at ambient temperature and resuspended in 5-10 mL of medium and a cell count was performed using a hemocytometer. The PBMCs were resuspended at 2×10⁶ cells/mL. DCs and PBMCs were recombined in the plate to stimulate CTL at a 1:10 to 1:5 ratio of DCs:CTL. Cytokines IL-7, IL-15, IL-6, and IL-12 were added to achieve a final concentration of IL-7 (10 ng/mL, R&D Systems)), IL-15 (5 ng/mL, CellGenix), IL-6 (100 ng/mL, CellGenix), and IL-12 (10 ng/mL, R&D Systems). All surrounding wells were filled with approximately 2 mL of PBS to maintain humidity within the plate. The cells were cultured in the incubator at 37° C. and 5% CO₂ for 7 to 8 days. A one-half medium change was performed on day 4 to 5, with the wells being split 1:1 if nearly confluent.

Step 4. Second T-Cell Stimulation in 24-Well Plate

The second stimulation of T-cells was performed using either overlapping peptide library-Pulsed Autologous DCs (Procedure A) or overlapping peptide library—Pulsed Autologous Phytohemagglutinin (PHA) Blasts (Procedure B) as antigen presenting cells.

Procedure A: Stimulation Using Overlapping Peptide Library—Pulsed Autologous DCs as Antigen Presenting Cells (APCs)

After pulsing with the appropriate overlapping peptide library (PRAME, WT1, and Survivin Pep Mix™; JPT Peptide Technologies), DCs were irradiated at 25 Gy. The DCs were washed with DC medium and centrifuged at 400×g for 5 minutes at ambient temperature. The supernatant was aspirated and the wash step was repeated twice more. The cells were counted using a hemocytometer. The DCs were resuspended at 0.5-2×10⁵ cells/mL of CTL medium with 10% HS (Valley) for initiation. Plate 1 mL of irradiated DCs/well (0.5-2×10⁵ cells) in a 24-well tissue culture treated plate. T-cells were counted using a hemocytometer. The cells were resuspended at 1×10⁶ cells/mL of T-cells medium supplemented with IL-7 (10 ng/mL final concentration, R&D Systems)) and IL-2 (100 U/mL final concentration, Proleukin) and 1 mL was aliquoted per well of the 24-well plate. The cells were cultured in the incubator at 37° C. and 5% CO₂ for 3 to 4 days. The medium was changed with IL-2 (˜100 U/mL final concentration, Proleukin) and cultured for another 3 to 4 days. Cells can be frozen after the second stimulation.

Procedure B: Stimulation Using Overlapping Peptide Library-Pulsed Autologous Phytohemagglutinin (PHA) Blasts as APCs

Autologous PHA blasts are harvested on day 7 by gentle resuspension, and cells were counted using a hemocytometer. The PHA blasts were transferred to a 15 mL centrifuge tube and centrifuged for 5 minutes at 400×g at ambient temperature. The Supernatant was aspirated and the pellet was resuspended by finger flicking. 100 μL of appropriate overlapping peptide library Mastermix (200 ng/peptide in 200 μL; PRAME, WT1, and Survivin Pep Mix™; JPT Peptide Technologies) was added to PHA blasts per 1-10×10⁶ cells. The PHA blasts were incubated for 30-60 minutes. The PHA blasts were resuspended in 5-10 mL of medium and irradiated at 50 Gy (or 100 Gy if used in G-rex). The PHA blasts were washed with CTL medium and centrifuged at 400×g for 5 minutes at ambient temperature. The supernatant was aspirated and the washing step was repeated twice more. A cell count was performed using a hemocytometer. The PHA blasts were resuspended at 0.5×10⁶ cell s/mL of CTL medium to re-stimulate T-cells at an approximate ratio of 1:1 PHA blasts:T-cell. The T-cells were counted using a hemocytometer. The T-cells were resuspended at 0.5×10⁶ cells/mL of CTL medium supplemented with IL-7 (100 ng/mL final concentration; R&D Systems) and IL-2 (100 U/mL final concentration; Proleukin). One well of only PHA blasts was maintained as an irradiation control. The cells were cultured in the incubator at 37° C. and 5% CO₂ for 3 to 4 days. The medium was changed with IL-2 (100 U/mL final concentration; Proleukin) and the cells are cultured for another 3 to 4 days.

Step 5. Third T-Cell Stimulation in G-Rex10 Using PHA Blasts as APCs

Autologous PHA blasts were harvested on day 7 by gentle resuspension, and cells were counted using a hemocytometer. The PHA blasts were transferred to a 15 mL centrifuge tube and centrifuged for 5 minutes at 400×g at ambient temperature. The supernatant was aspirated, and the pellet was resuspended by finger flicking. 100 μL of appropriate Overlapping Peptide Library Mastermix (200 ng/peptide in 200 μL; PRAME, WT1, and Survivin Pep Mix™; JPT Peptide Technologies) was added to PHA blasts per 1-10×10⁶ cells, and the PHA blasts were incubated for 30-60 minutes. The PHA blasts were resuspended in 5-10 mL of medium and irradiated at 50 Gy (or 100 Gy if used in G-Rex). The PHA blasts were washed with CTL medium and centrifuged at 400×g for 5 minutes at ambient temperature. The supernatant was aspirated, and the washing step was repeated twice more. Cells were counted using a hemocytometer. The PHA blasts were resuspended at 0.5×10⁶ cells/mL of CTL medium to re-stimulate T-cells at an approximate ratio of 1:1 PHA blasts. 10 mL of cell suspension was added in the G-Rex10 and 1 mL/well (0.5×10⁶ PHA blasts) in the 24-well control plate. The T-cells were counted using a hemocytometer. The T-cells are resuspended at 05×10⁶ cells/mL of CTL medium, and 10 mL (5×10⁶ CTLs) was added in the G-Rex10 and 1 mL/well (0.5×10⁶ CTLs) in the 24-well control plate. The medium was supplemented with IL-7 (10 ng/mL final concentration; R&D Systems) and IL-2 (100 U/mL final concentration; Proleukin), and the cells were cultured in the incubator at 37° C. and 5% CO₂ for 3 to 4 days. One well of the 24 well plate was left with PHA blasts only as an irradiation control. The medium was changed with IL-2 (100 U/mL final concentration; Proleukin), and the cells were cultured for an additional 3 to 4 days.

### Example 2. ELISPOT Plating and Development for Analysis of T-Cell Function

Peptide recognition for a multi-TAA specific to survivin, PRAME, and WT1 was tested in an IFN-γ-enzyme-linked immunospot (ELISpot) assay. Recognition of the pooled TAAs as well as single antigens was tested as compared with no-peptide media control (SEB 90%), CTL none, and actin. The results are shown in FIG. 2. The 3-day procedure for performing the ELISpot assay is detailed below.

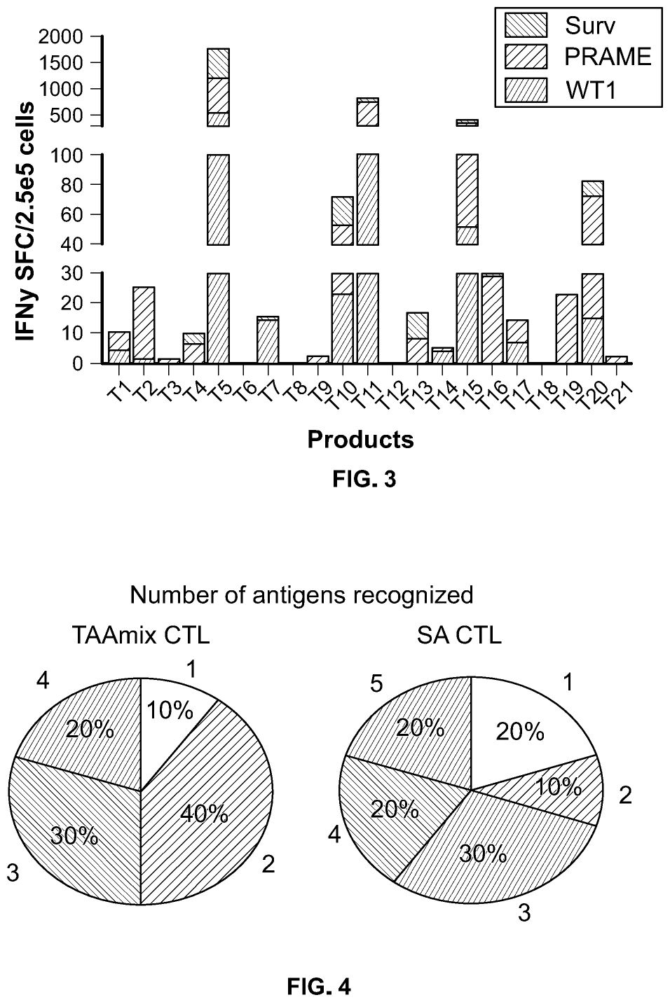

Additionally, peptide recognition for 21 T-cell populations generated from solid tumor patients was tested in an IFN-γ-enzyme-linked immunospot (ELISpot) assay following the same procedure as above. Recognition of the pooled TAAs as well as single antigens was tested as compared with no-peptide media control (SEB 90%), CTL none, and actin. Antigen specificity was demonstrated by IFN γ ELISpot assays after stimulation with tumor-associated antigens. All products showed response to SEB, the positive control. Actin was used as a negative control to detect non-specific activity. Specificity to actin was subtracted from the results for the peptides WT1, PRAME, and survivin. A positive was defined as result of 10 IFNγ SFC/2.5×10⁵ cells or greater following subtraction of actin. One product did not have specificity for any antigens, including actin. Half of the remaining products demonstrated high background activity to actin exceeding activity to specific TAAs. Overall the actin activity ranged 0-159.5 IFNγ SFC/2.5×10⁵ cells with a median of 23.25 IFNγ SFC/2.5×10⁵ cells. Response to specific antigens with actin subtracted out was as follows: WT1 (median 0.75, range 0-561 IFNγ SFC/2.5×10⁵ cells); PRAME (median 6.25, range 0-653.5 IFNγ SFC/2.5×10⁵ cells); survivin (median 0, range 0-540 IFNγ SFC/2.5×10⁵ cells). 17% of products demonstrated positivity as defined above for all 3 antigens, 11% of products for two antigens, 11% of products for 1 antigen; 61% of products did not demonstrate any specificity by ELISpot as defined by these criteria. The results of the ELISpot assay for the 21 TAA-L products are shown in FIG. 3.

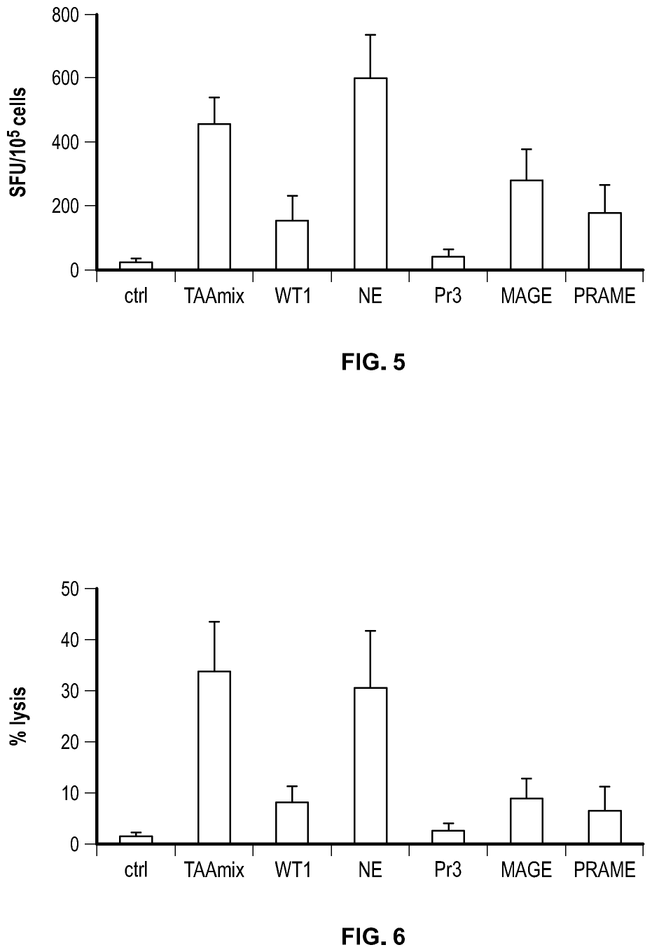

Additionally, Weber et al. generated a multi-TAA from 10 donors that was specific to five TAA: WT1, Pr3, NE, MAGE-A3 and PRAME. Recognition of the pooled TAAs as well as single antigens was tested as compared with no-peptide media control (SEB 90%), CTL none, and actin. The results are shown in FIGS. 4 and 5.

Day 1: ELISPOT Plate Preparation

ELISPOT coating buffer is prepared by dissolving 1.59 g Na₂CO₃ to one liter of sterile water followed by sterile filtration. INFγ-capture antibody (Ab) solution is prepared by added 100 IFN-γ mAB 1-D1K (MabTech) to every 10 mL ELISPOT coating buffer. 35 μL of 70% ethanol is added to each well of a 96-well filtration plate (Millipore) using a 200 μL multichannel pipette. The ethanol is dumped, and the plate is immediately washed two times with 150 μL PBS. The last PBS wash is dumped, and 100 μL of Ab solution is immediately added to each well. The plate edges are wrapped in parafilm to prevent evaporation, and the plates sit for a minimum of 6 hours at 4° C. These coated plates are stable at 4° C. for up to 4 weeks.

Day 2: ELISPOT Cell Plating

ELISPOT media is prepared by combining 250 mL RPMI, 12.5 mL human serum (HS), and 2.5 mL sterile-filtered GlutaMAX. The coating buffer in the 96-well plate is dumped, and the wells are washed two times with 150 μL PBS. 100 μL of ELISPOT media is added to each well, and the plate is placed in the incubator at 37° C. for a minimum of one hour.

While the plate is incubating, peptide pools are prepared in a 24-well plate. The following peptide pools are prepared using 250 μL of ELISPOT media and 2.5 μL peptide: PBMC; Actin; Staphylococcal enterotoxin B (SEB; dosed at 1.0 μL peptide); PRAME; Survivin; WT1; and TAA (PRAME+Survivin+WT1). The cells are harvested and counted using a hemocytometer. 4.0×10⁶ cells are aliquoted and centrifuged at 400×g for 5 minutes and supernatant removed. The cells are resuspended in ELISPOT media to ensure 2.5×10⁵ cell/100 μL media. The ELISPOT media is dumped from the plate after incubation, and 100 μL of cells are placed in the appropriate wells. 100 μL of peptide pool is mixed in the appropriate wells and incubated at 37° C. overnight.

Day 3: ELISPOT Plate Development

The cells are decanted from the plate, and the plate is washed six times with PBS/0.05% Tween 20 solution. Biotin buffer is prepared by adding 2.5 g bovine serum albumin (BSA) powder to 500 mL PBS followed by sterile filtering. The biotinylated antibody solution is prepared by adding 10 μL mAb 7-B6 (MabTech) to every 10 mL of Biotin buffer. The last plate wash is decanted and 100 μL of biotinylated antibody solution is added to each well. The plate is incubated at 37° C. for 1 to 2 hours. The biotinylated antibody solution is decanted, and the plate is washed six times with PBS/0.05% Tween 20 solution. 100 μL of Avidin-Peroxidase Complex (APC) solution is added to each well using a multichannel pipette. The plate is covered with foil and sat at room temperature for 1 to 2 hours. The 3-amino-9-ethylcarbazole (AEC) substrate solution is prepared while the plate is incubating by dissolving the AEC tablet in 2.5 mL of dimethylformamide in a 50 mL centrifuge tube, adding 47.5 mL acetate buffer (prepared by mixing 4.6 mL 0.1 N acetic acid, 11 mL 0.1 M sodium acetate, and 46.9 mL sterile water) and 25 hydrogen peroxide, and mixing by inverting. The APC solution is decanted, and the plate is washed three times with plain PBS solution. 100 μL of AEC substrate solution is then added to each well, the plate is covered in foil, and incubated for 4 minutes. The AEC solution is decanted, and plate development is halted by rinsing with vigorously running water. The plate backing is removed, the membranes are rinsed with water, and the plate is firmly tapped against a paper towel to remove any excess water. The plates are dried by placing them upside down with no lip on a hood grate. Upon drying, the plates are wrapped in paper towel and stored in a dark place to prevent bleaching of spots.

Spot-forming cells (SFCs) were counted and evaluated using an automated plate reader system (Karl Zeiss).

### Example 3. Cytotoxicity Assay to Measure Cytolytic Activity of Multi-TAA-Specific T-Cell Populations

The cytolytic activity of the multi-TAA-specific T-cell populations from Weber et al. was also measured in a cytotoxicity assay. Target cells were fluorescence labeled with carboxyfluorescein succinimidyl ester (CFSE, Invitrogen), peptide-pulsed and incubated with T-cells at different effector-to-target ratios for 6-8 hrs. Ethidium homodimer (Invitrogen) was added after incubation to stain dead cells. Samples were acquired on a BD Fortessa Flow Cytometer. The number of live target cells was determined by gating on carboxyfluorescein succinimidyl ester-positive, ethidium homodimer-negative cells, and used to calculate cytolytic activity as follows: Lysis (%)=100−((live target cells/sample/live target cells control)×100). The results of the cytotoxicity assay are shown in FIG. 6.

### Example 4. Antileukemic Activity Against Partially HLA-Matched AML Blasts

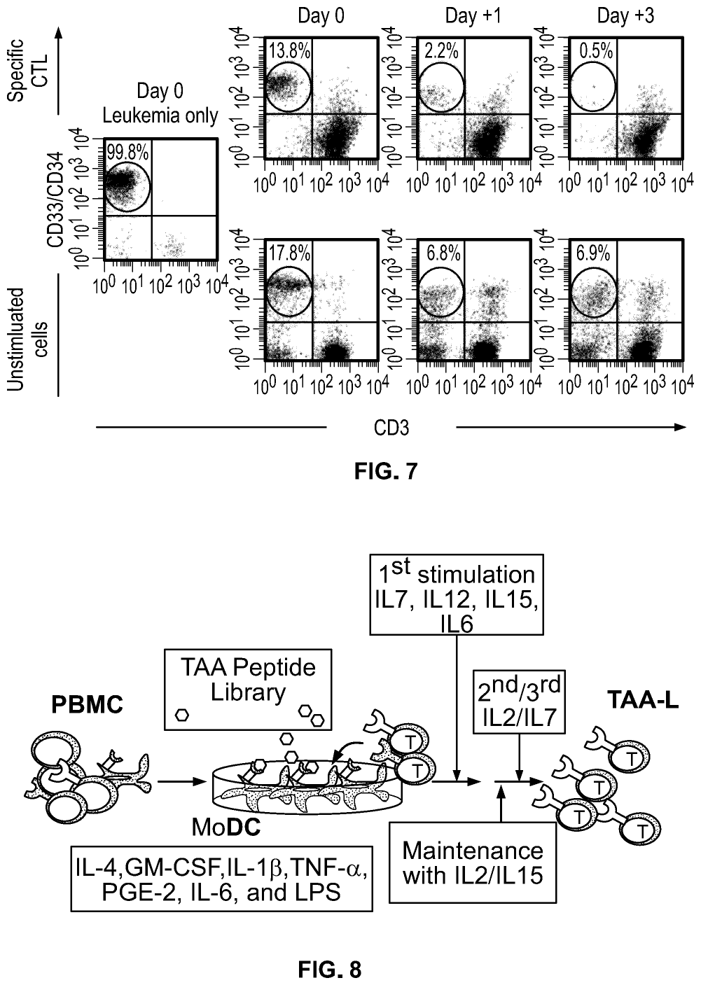

To evaluate the antileukemic activity of multi-TAAmix-specific T-cells in vitro, T-cells were cocultured with primary leukemia blast samples matched in at least one HLA-antigen (range 1-3), including pairs, which were matched solely at HLA class II alleles. Where available, AML blast samples were evaluated for expression of MAGE-A3 and PRAME. Low expression was detectable by immunohistochemistry (data not shown). FIG. 7 shows the results of five T-cell lines tested against a panel of five primary AML blast samples. Specific leukemia recognition and elimination occurred even with single HLA class I or II allele matched targets. As control for nonspecific lysis or allogeneic reactivity, cytotoxic T-cell lines with irrelevant specificity (viral antigens) generated from the same donor were used in all experiments. All donor-derived multi-TAAmix-specific T-cells showed leukemia-specific killing, as well as T-cell activation as determined by CFU assay, IFNγ-ELISpot, and intracellular cytokine detection. A representative example of a coculture experiment with AML blast is shown in FIG. 7. A 3-day coculture with specific cytotoxic T-cells eliminated leukemia blasts (0.5%), compared with 6% blasts persisting in cocultures with control cytotoxic T-cells.

### Example 5. Comparison of Antigen Activity of T-Cell Compositions Generated by Multi-TAA, Single TAA, and Equal Ratio Combination of Single TAA Compositions (MUSTANG Composition)

Mononuclear cell products were provided by a donor. Multi-TAA T-cell populations were generated using the process outlined in Example 1 and shown in FIG. 8. Specifically, Peripheral blood mononuclear cells (PBMCs) were isolated from whole blood or leukopaks using Lymphoprep density gradient separation. PBMCs were washed with PBS 1×, counted and immediately used to generate dendritic cells (DCs) or were frozen at 50-100×10⁶ cells/mL/vial for future use. Monocytes were isolated by adherence in 6 well plates by incubating 20×10⁶ PBMCs/well in CellGenix medium for 1-2 hrs and then were washed 3 times with CellGenix to remove non adherent cells (NACs). NACs were then counted and frozen at 20-50×10⁶ cells/mL/vial for future use. DCs were generated by culturing monocytes for 6-9 days in CellGenix medium supplemented with 1000 U/mL of IL-4 and 800 U/mL of GM-CSF and were then matured for 24 hrs in CellGenix supplemented with 1000 U/mL of IL-4, 800 U/mL of GM-CSF, 10 ng/mL of TNF-α, 100 ng/mL of IL-6, 10 ng/mL of IL-1β and 30 ng/mL of LPS. Matured DCs were harvested by scraping, counted, equally split into 4 tubes and exposed to 4 different peptide mixes (Pep Mixes™ WT1, Prame, Survivin or all together TAA) for 1 hr, before being irradiated. PBMCs or NACs were then initiated with DCs in a 24 well plate at a ratio of 1:5 (DC:PBMC/NAC) at a maximum of 2×10⁶ cells per well in 2 mL of CTL medium (45% advanced RPMI, 45% Click's medium, 10% human serum and 1% glutamax) supplemented with 10 ng/mL IL-7, 5 ng/mL IL-15, 10 ng/mL IL-12 and 100 ng/mL IL-6 and cultured for 7-8 days. Harvested cells were counted and initiated a second time with irradiated matured DCs exposed to PepMixes™ at a ratio of 1:2 to 1:48 (DCs:CTL) in CTL medium supplemented with 10 ng/mL of IL-7 and 100 U/mL of IL-2. CTLs were cultured an additional 7 days and were harvested for analysis or initiated a third time with phytohaemagglutinin blasts (PHA blasts). PHA blasts were generated with 10×10⁶ thawed NACs exposed to 5μg/mL PHA for 24 hrs in a 24 well plate at 10⁶ cells/well/2 mL CTL medium. The day after, PHA blasts were harvested, counted and exposed to the same PepMixes™ (WT1, Prame, Survivin and TAA) for 1 hr before irradiation. CTLs from the second initiation (5×10⁶ cells) were cultured with irradiated PHA blasts at a ratio of 1:8 to 1:18 (PHA blast:CTL). Lines were cultured in GRex in 20 mL CTL medium supplemented with 10 ng/mL of IL-7 and 100 U/mL of IL-2 for 7 days and were finally harvested for analysis. Equal amounts of single antigen TAA overlapping peptide libraries (WT1, PRAME, and Survivin) were mixed together and used to prime APCs and aAPCs to generate the multi-TAA T-cell composition. Single TAA T-cell subpopulations were generated using the process outlined in Example 1 and shown in FIG. 8. Single antigen overlapping peptide libraries (WT1, PRAME, and Survivin) were used to prime APCs and aAPCs to generate the single-TAA T-cell compositions. Single TAA T-cell compositions were mixed together in a 1:1:1 ratio. Each of the resultant T-cell compositions were tested for antigen specificity utilizing the EliSpot Assay, the process of which is outlined in Example 2. The results are shown in FIG. 10 as a log of spot forming units (SFU) per 10⁵ cells and is normalized to actin, which is the positive control. The product generated using the multi-TAA methodology only showed activity to two of the antigens (WT1 and PRAME) whereas the 1:1:1 mix of the single antigen T-cell subpopulations showed activity to all three of the antigens (WT1, PRAME, and Survivin).

The activity data generated for each T-cell subpopulation can be used to ensure that the activity of each T-cell subpopulation is equal. As shown in FIG. 10, the PRAME activity of the MUSTANG composition described in Example 5 is approximately 10-fold greater than the activity of WT1 and Survivin. To control for the level of activity in the MUSTANG composition differing amounts of the T-cell subpopulations can be combined to create a MUSTANG composition that has roughly equal activity across all antigens if desired, or also used to create variable ratios based on the expression profile of the patient's tumor. In order to generate a MUSTANG composition that has equal activity for all three antigens it would be necessary to combine the WT1, Survivin, and PRAMS T-cell subpopulations in a 10:10:1 ratio. This ratio can be adjusted based on the desired activity of each T-cell subpopulation in the final MUSTANG composition. Additionally, the in vitro anti-tumor activity of the MUSTANG composition can be determined using the process described in Example 4. Additional characterization of the TAA CTLs include identification of epitopes with TAA activity, determining the HLA restriction response, and performing a HLA restriction assay to determine antigen activity through a corresponding allele. To determine the composition, a blood or biopsy sample of the patient is provided which is used to determine the HLA subtype and antigen expression profile of a subject with a hematological malignancy or tumor. The MUSTANG composition is selected from the available single-TAA CTLs based on the highest antigen specificity through shared alleles. A schematic showing this process is shown in FIG. 9.

This specification has been described with reference to embodiments of the invention. The invention has been described with reference to assorted embodiments, which are illustrated by the accompanying Examples. The invention can, however, be embodied in different forms and should not be construed as limited to the embodiments set forth herein. Given the teaching herein, one of ordinary skill in the art will be able to modify the invention for a desired purpose and such variations are considered within the scope of the invention.

1. An isolated T-cell composition for administration to a patient with a tumor comprising two or more T-cell subpopulations,
wherein each T-cell subpopulation is specific for a single tumor associated antigen;
wherein each of the T-cell subpopulations is specific for a different tumor associated antigen;
wherein each of the T-cell subpopulations are primed and/or expanded separately from each other;
wherein each of the T-cell subpopulations are primed and/or expanded ex vivo; and
wherein each of the T-cell subpopulations are combined in the T-cell composition in a defined ratio, wherein the defined ratio is either total cell number or normalized cell activity.

2. The isolated T-cell composition of claim 1,
wherein each of the T-cell subpopulations is derived from an allogeneic donor cell source;
wherein each of the T-cell subpopulations is primed and/or expanded using a group of peptides comprising peptides specific to each tumor associated antigen that are HLA restricted epitopes to one or more HLA alleles of the donor cell source.

3. The isolated T-cell composition of claim 2,
wherein the HLA allele is HLA-A, HLA B, and/or HLA-C.

4. The T-cell composition of claim 1, wherein the defined ratio of each of the T-cell subpopulations in the T-cell composition is based on the total number of cells of each T-cell subpopulation.

5. The T-cell composition of claim 1, wherein the defined ratio of each of the T-cell subpopulations in the T-cell composition is based on the normalized cell activity of each T-cell subpopulation.

6. The T-cell composition of claim 1, wherein the defined ratio of each of the T-cell subpopulations in the T-cell composition is about equal.

7. The T-cell composition of claim 1, wherein each of the T-cell subpopulations is specific for a tumor associated antigen expressed by the patient's tumor.

8. The T-cell composition of claim 7, wherein one or more of the single-tumor associated antigens is selected from the group consisting of WT1, PRAME, Survivin, NY-ESO-1, MAGE-A3, MAGE-A4, Pr3, XBP1, CD138, CS1, Cyclin A1, SSX2, Neutrophil Elastase (NE), HPV E6. HPV E7, EBV LMP1, EBV LMP2, EBV EBNA1, or EBV EBNA2.

9. The T-cell composition of claim 1, wherein the T-cell composition comprises at least two T-cell subpopulations.

10. The T-cell composition of claim 9, wherein the composition comprises at least three T-cell subpopulations having at least about 45% of the first T-cell subpopulation, at least about 10% of the second T-cell subpopulation, and at least about 5% of the third T-cell subpopulation.

11-14. (canceled)

15. The T-cell composition of claim 1, wherein the tumor is a hematological malignancy.

16-19. (canceled)

20. The T-cell composition of claim 9, wherein the tumor associated antigens are PRAME, WT1, and survivin.

21-24. (canceled)

25. The T-cell composition of claim 20, wherein the composition comprises at least three T-cell subpopulations and wherein the first T-cell subpopulation is specific for PRAME, the second T-cell subpopulation is specific for WT1, and the third T-cell subpopulation is specific for survivin.

26. The T-cell composition of claim 1, wherein one or more of the T-cell subpopulations is derived from cord blood and/or stem cells.

27. The T-cell composition of claim 2, wherein the HLA allele is HLA-A, HLA-B, and/or HLA-DR.

28-67. (canceled)

68. A method of treating a patient with a tumor comprising:
i) determining the HLA subtype of the patient;
ii) diagnosing the tumor type of the patient;
iii) identifying two or more tumor associated antigens associated with the tumor type for targeting with tumor associated antigen-specific T-cell subpopulations;
iv) selecting one banked T-cell subpopulation having activity against each targeted tumor associated antigen through one or more HLA-alleles shared between the patient and the T-cell subpopulations, wherein each T-cell subpopulation is specific for a single tumor associated antigen, wherein each of the T-cell subpopulations is specific for a different tumor associated antigen, wherein each of the T-cell subpopulations are primed and expanded separately from each other, wherein each of the T-cell subpopulations are primed and expanded ex vivo;
v) combining each selected banked T-cell subpopulation to create a T-cell composition; and,
vi) administering an effective amount of the T-cell composition to the patient.

69-70. (canceled)

71. A method of treating a patient with a tumor comprising:
i) determining the HLA subtype of the patient;
ii) diagnosing the tumor type of the patient;
iii) identifying a pre-selected set of two or more tumor associated antigens associated with the tumor type for targeting with tumor associated antigen-specific T-cell subpopulations;
iv) selecting one banked T-cell subpopulation having activity against each targeted tumor associated antigen through one or more HLA-alleles shared between the patient and the T-cell subpopulations, wherein each T-cell subpopulation is specific for a single tumor associated antigen, wherein each of the T-cell subpopulations is specific for a different tumor associated antigen, wherein each of the T-cell subpopulations are primed and expanded separately from each other, wherein each of the T-cell subpopulations are primed and expanded ex vivo;
v) combining each selected banked T-cell subpopulation to create a first T-cell composition;
vi) administering an effective amount of the first T-cell composition to the patient;
vii) monitoring the patient's response to the first T-cell composition by measuring the presence of circulating TAA specific T-cells;
viii) monitoring changes to the patient's tumor associated antigen expression profile;
ix) if the patient's tumor associated antigen expression profile has changed, identifying two or more tumor associated antigens expressed by the patient's tumor for targeting with tumor associated antigen-specific T-cell subpopulations, wherein if the patient is showing a robust response to any specific tumor associated antigen T-cell subpopulation(s) from the first T-cell composition, exclude targeting that tumor associated antigen;
x) selecting one banked T-cell subpopulation having the highest activity against each targeted tumor associated from step ix) through one or more HLA-alleles shared between the patient and the T-cell subpopulations, wherein each T-cell subpopulation is specific for a single tumor associated antigen, wherein each of the T-cell subpopulations is specific for a different tumor associated antigen, wherein each of the T-cell subpopulations are primed and expanded separately from each other, wherein each of the T-cell subpopulations are primed and expanded ex vivo;
xi) combining each selected banked T-cell subpopulation to create a second T-cell composition;
xii) administering an effective amount of the second T-cell composition to the patient; and
xiii) optionally repeating steps viii) to xii); and,
xiv) combining each selected banked T-cell subpopulation to create a third T-cell composition; and,
xv) administering an effective amount of the third T-cell composition to the patient.

72. The method of claim 68, wherein the T-cell composition administered to the patient is tested for reactivity to the patient's tumor prior to administration.

73. The method of claim 68, wherein the T-cell composition is derived from an allogeneic donor.

74-81. (canceled)

82. The T-cell composition of claim 1, wherein one or more T-cell subpopulations is primed and/or expanded with an overlapping peptide library.

83. The T-cell composition of claim 82, wherein one or more T-cell subpopulations is primed and/or expanded with both an overlapping peptide library and a group of peptides comprising peptides specific to each tumor associated antigen that are HLA-restricted to at least one of the donor's HLA-A alleles, at least one of the donor's HLA-B alleles, and at least one of the donor's HLA-DR alleles.

84-86. (canceled)

87. The method of claim 71, wherein the T-cell composition administered to the patient is tested for reactivity to the patient's tumor prior to administration.

88. The method of claim 71, wherein the T-cell composition is derived from an allogeneic donor.
